SEQUENCE LISTING (1204 sequences)

SEQ ID NO: 1 - protein, 6 aa, Homo sapiens
VTNFRS

SEQ ID NO: 2 - protein, 7 aa, Homo sapiens
LTSSGIE

SEQ ID NO: 3 - protein, 14 aa, Homo sapiens
GGLNAGGTSYGKLT

SEQ ID NO: 4 - protein, 114 aa, Homo sapiens
EDKVVQSPLSLVVHEGDTVTLNCSYEVTNFRSLLWYKQEKKAPTFLFMLTSSGIEKKSGRLSSILDKKELFSILNITATQTGDSAIYLCGGLNAGGTSYGKLTFGQGTILTVHP

SEQ ID NO: 5 - protein, 135 aa, Homo sapiens
MMKCPQALLAIFWLLLSWVSSEDKVVQSPLSLVVHEGDTVTLNCSYEVTNFRSLLWYKQEKKAPTFLFMLTSSGIEKKSGRLSSILDKKELFSILNITATQTGDSAIYLCGGLNAGGTSYGKLTFGQGTILTVHP

SEQ ID NO: 6 - protein, 272 aa, synthetic construct
MMKCPQALLAIFWLLLSWVSSEDKVVQSPLSLVVHEGDTVTLNCSYEVTNFRSLLWYKQEKKAPTFLFMLTSSGIEKKSGRLSSILDKKELFSILNITATQTGDSAIYLCGGLNAGGTSYGKLTFGQGTILTVHPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 7 - protein, 5 aa, Homo sapiens
SNHLY

SEQ ID NO: 8 - protein, 6 aa, Homo sapiens
FYNNEI

SEQ ID NO: 9 - protein, 11 aa, Homo sapiens
ASLGASTYEQY

SEQ ID NO: 10 - protein, 113 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFCASLGASTYEQYFGPGTRLTVT

SEQ ID NO: 11 - protein, 132 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASLGASTYEQYFGPGTRLTVT

SEQ ID NO: 12 - protein, 305 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASLGASTYEQYFGPGTRLTVTEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 13 - protein, 6 aa, Homo sapiens
VSGNPY

SEQ ID NO: 14 - protein, 8 aa, Homo sapiens
YITGDNLV

SEQ ID NO: 15 - protein, 11 aa, Homo sapiens
AVRDDYGQNFV

SEQ ID NO: 16 - protein, 114 aa, Homo sapiens
DTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLSGPSYEQYFGPGTRLTVT

SEQ ID NO: 17 - protein, 133 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLSGPSYEQYFGPGTRLTVT

SEQ ID NO: 18 - protein, 270 aa, synthetic construct
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLSGPSYEQYFGPGTRLTVTNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 19 - protein, 5 aa, Homo sapiens
SEHNR

SEQ ID NO: 20 - protein, 6 aa, Homo sapiens
FQNEAQ

SEQ ID NO: 21 - protein, 12 aa, Homo sapiens
ASSLSGPSYEQY

SEQ ID NO: 22 - protein, 112 aa, Homo sapiens
QSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDDYGQNFVFGPGTRLSVLP

SEQ ID NO: 23 - protein, 132 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDDYGQNFVFGPGTRLSVLP

SEQ ID NO: 24 - protein, 305 aa, synthetic construct
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDDYGQNFVFGPGTRLSVLPEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 25 - protein, 6 aa, Homo sapiens
DSAIYN

SEQ ID NO: 26 - protein, 7 aa, Homo sapiens
IQSSQRE

SEQ ID NO: 27 - protein, 11 aa, Homo sapiens
AVRHSGNTPLV

SEQ ID NO: 28 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRHSGNTPLVFGKGTRLSVIA

SEQ ID NO: 29 - protein, 131 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRHSGNTPLVFGKGTRLSVIA

SEQ ID NO: 30 - protein, 268 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRHSGNTPLVFGKGTRLSVIANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 31 - protein, 6 aa, Homo sapiens
DFQATT

SEQ ID NO: 32 - protein, 7 aa, Homo sapiens
SNEGSKA

SEQ ID NO: 33 - protein, 11 aa, Homo sapiens
SASGGGRTEAF

SEQ ID NO: 34 - protein, 114 aa, Homo sapiens
AVVSQHPSRVICKSGTSVKIECRSLDFQATTMFWYRQFPKQSLMLMATSNEGSKATYEQGVEKDKFLINHASLTLSTLTVTSAHPEDSSFYICSASGGGRTEAFFGQGTRLTVV

SEQ ID NO: 35 - protein, 129 aa, Homo sapiens
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSASGGGRTEAFFGQGTRLTVV

SEQ ID NO: 36 - protein, 302 aa, synthetic construct
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSASGGGRTEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 37 - protein, 6 aa, Homo sapiens
DSAIYN

SEQ ID NO: 38 - protein, 7 aa, Homo sapiens
IQSSQRE

SEQ ID NO: 39 - protein, 12 aa, Homo sapiens
AVRRDGTASKLT

SEQ ID NO: 40 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRRDGTASKLTFGTGTRLQVTL

SEQ ID NO: 41 - protein, 132 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRRDGTASKLTFGTGTRLQVTL

SEQ ID NO: 42 - protein, 269 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRRDGTASKLTFGTGTRLQVTLNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 43 - protein, 6 aa, Homo sapiens
DFQATT

SEQ ID NO: 44 - protein, 7 aa, Homo sapiens
SNEGSKA

SEQ ID NO: 45 - protein, 12 aa, Homo sapiens
SASFPGRGNEQF

SEQ ID NO: 46 - protein, 115 aa, Homo sapiens
AVVSQHPSWVICKSGTSVKIECRSLDFQATTMFWYRQFPKQSLMLMATSNEGSKATYEQGVEKDKFLINHASLTLSTLTVTSAHPEDSSFYICSASFPGRGNEQFFGPGTRLTVL

SEQ ID NO: 47 - protein, 130 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSASFPGRGNEQFFGPGTRLTVL

SEQ ID NO: 48 - protein, 303 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSASFPGRGNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 49 - protein, 6 aa, Homo sapiens
NTAFDY

SEQ ID NO: 50 - protein, 7 aa, Homo sapiens
IRPDVSE

SEQ ID NO: 51 - protein, 13 aa, Homo sapiens
AASRASGGSYIPT

SEQ ID NO: 52 - protein, 121 aa, Homo sapiens
QQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASRASGGSYIPTFGRGTSLIVHP

SEQ ID NO: 53 - protein, 142 aa, Homo sapiens
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASRASGGSYIPTFGRGTSLIVHP

SEQ ID NO: 54 - protein, 279 aa, synthetic construct
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASRASGGSYIPTFGRGTSLIVHPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 55 - protein, 5 aa, Homo sapiens
LNHDA

SEQ ID NO: 56 - protein, 6 aa, Homo sapiens
SQIVND

SEQ ID NO: 57 - protein, 14 aa, Homo sapiens
ATKILAGANTGELF

SEQ ID NO: 58 - protein, 113 aa, Homo sapiens
GITQSPKYLFRKEGQNVTLSCEQNLNHDAMYWYRQDPGQGLRLIYYSQIVNDFQKGDIAEGYSVSREKKESFPLTVTSAQKNPTAFYLCATKILAGANTGELFFGEGSRLTVL

SEQ ID NO: 59 - protein, 134 aa, Homo sapiens
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCATKILAGANTGELFFGEGSRLTVL

SEQ ID NO: 60 - protein, 307 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCATKILAGANTGELFFGEGSRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 61 - protein, 5 aa, Homo sapiens
VGISA

SEQ ID NO: 62 - protein, 5 aa, Homo sapiens
LSSGK

SEQ ID NO: 63 - protein, 18 aa, Homo sapiens
AAGGPQLAPKETSGSRLT

SEQ ID NO: 64 - protein, 117 aa, Homo sapiens
AKNEVEQSPQNLTAQEGEFITINCSYSVGISALHWLQQHPGGGIVSLFMLSSGKKKHGRLIATINIQEKHSSLHITASHPRDSAVYICAAGGPQLAPKETSGSRLTFGEGTQLTVNP

SEQ ID NO: 65 - protein, 138 aa, Homo sapiens
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAAGGPQLAPKETSGSRLTFGEGTQLTVNP

SEQ ID NO: 66 - protein, 275 aa, synthetic construct
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAAGGPQLAPKETSGSRLTFGEGTQLTVNPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 67 - protein, 5 aa, Homo sapiens
SGHRS

SEQ ID NO: 68 - protein, 6 aa, Homo sapiens
YFSETQ

SEQ ID NO: 69 - protein, 12 aa, Homo sapiens
ASSLDPSGTGYT

SEQ ID NO: 70 - protein, 111 aa, Homo sapiens
GVTQTPRYLIKTRGQQVTLSCSPISGHRSVSWYQQTPGQGLQFLFEYFSETQRNKGNFPGRFSGRQFSNSRSEMNVSTLELGDSALYLCASSLDPSGTGYTFGSGTRLTVV

SEQ ID NO: 71 - protein, 132 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLDPSGTGYTFGSGTRLTVV

SEQ ID NO: 72 - protein, 305 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLDPSGTGYTFGSGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 73 - protein, 6 aa, Homo sapiens
NSASQS

SEQ ID NO: 74 - protein, 6 aa, Homo sapiens
VYSSGN

SEQ ID NO: 75 - protein, 10 aa, Homo sapiens
VVKNQGGKLI

SEQ ID NO: 76 - protein, 110 aa, Homo sapiens
QRKEVEQDPGPFNVPEGATVAFNCTYSNSASQSFFWYRQDCRKEPKLLMSVYSSGNEDGRFTAQLNRASQYISLLIRDSKLSDSATYLCVVKNQGGKLIFGQGTELSVKP

SEQ ID NO: 77 - protein, 130 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVKNQGGKLIFGQGTELSVKP

SEQ ID NO: 78 - protein, 267 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVKNQGGKLIFGQGTELSVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 79 - protein, 5 aa, Homo sapiens
SGHRS

SEQ ID NO: 80 - protein, 6 aa, Homo sapiens
YFSETQ

SEQ ID NO: 81 - protein, 13 aa, Homo sapiens
ASILGGGRGDTQY

SEQ ID NO: 82 - protein, 112 aa, Homo sapiens
GVTQTPRYLIKTRGQQVTLSCSPISGHRSVSWYQQTPGQGLQFLFEYFSETQRNKGNFPGRFSGRQFSNSRSEMNVSTLELGDSALYLCASILGGGRGDTQYFGPGTRLTVL

SEQ ID NO: 83 - protein, 133 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASILGGGRGDTQYFGPGTRLTVL

SEQ ID NO: 84 - protein, 306 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASILGGGRGDTQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 85 - protein, 5 aa, Homo sapiens
SVFSS

SEQ ID NO: 86 - protein, 7 aa, Homo sapiens
VVTGGEV

SEQ ID NO: 87 - protein, 11 aa, Homo sapiens
AGDRPGNTPLV

SEQ ID NO: 88 - protein, 110 aa, Homo sapiens
QLLEQSPQFLSIQEGENLTVYCNSSSVFSSLQWYRQEPGEGPVLLVTVVTGGEVKKLKRLTFQFGDARKDSSLHITAAQPGDTGLYLCAGDRPGNTPLVFGKGTRLSVIA

SEQ ID NO: 89 - protein, 129 aa, Homo sapiens
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGDRPGNTPLVFGKGTRLSVIA

SEQ ID NO: 90 - protein, 129 aa, synthetic construct; identical to SEQ ID NO: 89

SEQ ID NO: 91 - protein, 5 aa, Homo sapiens
SGHTA

SEQ ID NO: 92 - protein, 6 aa, Homo sapiens
FQGNSA

SEQ ID NO: 93 - protein, 13 aa, Homo sapiens
ASSLSQGSSYEQY

SEQ ID NO: 94 - protein, 113 aa, Homo sapiens
GVSQSPSNKVTEKGKDVELRCDPISGHTALYWYRQSLGQGLEFLIYFQGNSAPDKSGLPSDRFSAERTGGSVSTLTIQRTQQEDSAVYLCASSLSQGSSYEQYFGPGTRLTVT

SEQ ID NO: 95 - protein, 134 aa, Homo sapiens
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLSQGSSYEQYFGPGTRLTVT

SEQ ID NO: 96 - protein, 307 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLSQGSSYEQYFGPGTRLTVTEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 97 - protein, 7 aa, Homo sapiens
TISGNEY

SEQ ID NO: 98 - protein, 5 aa, Homo sapiens
GLKNN

SEQ ID NO: 99 - protein, 14 aa, Homo sapiens
IVRVGTSNSGYALN

SEQ ID NO: 100 - protein, 111 aa, Homo sapiens
KTTQPTSMDCAEGRAANLPCNHSTISGNEYVYWYRQIHSQGPQYIIHGLKNNETNEMASLIITEDRKSSTLILPHATLRDTAVYYCIVRVGTSNSGYALNFGKGTSLLVTP

SEQ ID NO: 101 - protein, 130 aa, Homo sapiens
MRLVARVTVFLTFGTIIDAKTTQPTSMDCAEGRAANLPCNHSTISGNEYVYWYRQIHSQGPQYIIHGLKNNETNEMASLIITEDRKSSTLILPHATLRDTAVYYCIVRVGTSNSGYALNFGKGTSLLVTP

SEQ ID NO: 102 - protein, 267 aa, synthetic construct
MRLVARVTVFLTFGTIIDAKTTQPTSMDCAEGRAANLPCNHSTISGNEYVYWYRQIHSQGPQYIIHGLKNNETNEMASLIITEDRKSSTLILPHATLRDTAVYYCIVRVGTSNSGYALNFGKGTSLLVTPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 103 - protein, 5 aa, Homo sapiens
SGHTA

SEQ ID NO: 104 - protein, 6 aa, Homo sapiens
FQGNSA

SEQ ID NO: 105 - protein, 14 aa, Homo sapiens
ASSWVVGLSTDTQY

SEQ ID NO: 106 - protein, 114 aa, Homo sapiens
GVSQSPSNKVTEKGKDVELRCDPISGHTALYWYRQSLGQGLEFLIYFQGNSAPDKSGLPSDRFSAERTGGSVSTLTIQRTQQEDSAVYLCASSWVVGLSTDTQYFGPGTRLTVL

SEQ ID NO: 107 - protein, 135 aa, Homo sapiens
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSWVVGLSTDTQYFGPGTRLTVL

SEQ ID NO: 108 - protein, 308 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSWVVGLSTDTQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 109 - protein, 5 aa, Homo sapiens
TTLSN

SEQ ID NO: 110 - protein, 7 aa, Homo sapiens
LVKSGEV

SEQ ID NO: 111 - protein, 10 aa, Homo sapiens
AGRGGGFKTI

SEQ ID NO: 112 - protein, 109 aa, Homo sapiens
QQVMQIPQYQHVQEGEDFTTYCNSSTTLSNIQWYKQRPGGHPVFLIQLVKSGEVKKQKRLTFQFGEAKKNSSLHITATQTTDVGTYFCAGRGGGFKTIFGAGTRLFVKA

SEQ ID NO: 113 - protein, 128 aa, Homo sapiens
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGRGGGFKTIFGAGTRLFVKA

SEQ ID NO: 114 - protein, 265 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGRGGGFKTIFGAGTRLFVKANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 115 - protein, 5 aa, Homo sapiens
SGHTA

SEQ ID NO: 116 - protein, 6 aa, Homo sapiens
FQGNSA

SEQ ID NO: 117 - protein, 16 aa, Homo sapiens
ASSLRVMAGSNTGELF

SEQ ID NO: 118 - protein, 116 aa, Homo sapiens
GVSQSPSNKVTEKGKDVELRCDPISGHTALYWYRQSLGQGLEFLIYFQGNSAPDKSGLPSDRFSAERTGGSVSTLTIQRTQQEDSAVYLCASSLRVMAGSNTGELFFGEGSRLTVL

SEQ ID NO: 119 - protein, 137 aa, Homo sapiens
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRVMAGSNTGELFFGEGSRLTVL

SEQ ID NO: 120 - protein, 310 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLRVMAGSNTGELFFGEGSRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 121 - protein, 5 aa, Homo sapiens
DSVNN

SEQ ID NO: 122 - protein, 5 aa, Homo sapiens
IPSGT

SEQ ID NO: 123 - protein, 11 aa, Homo sapiens
AVSMDSSYKLI

SEQ ID NO: 124 - protein, 108 aa, Homo sapiens
IQVEQSPPDLILQEGANSTLRCNFSDSVNNLQWFHQNPWGQLINLFYIPSGTKQNGRLSATTVATERYSLLYISSSQTTDSGVYFCAVSMDSSYKLIFGSGTRLLVRP

SEQ ID NO: 125 - protein, 129 aa, Homo sapiens
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVSMDSSYKLIFGSGTRLLVRP

SEQ ID NO: 126 - protein, 266 aa, synthetic construct
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVSMDSSYKLIFGSGTRLLVRPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 127 - protein, 5 aa, Homo sapiens
LGHDT

SEQ ID NO: 128 - protein, 6 aa, Homo sapiens
YNNKEL

SEQ ID NO: 129 - protein, 12 aa, Homo sapiens
ASNDRGRRTEAF

SEQ ID NO: 130 - protein, 108 aa, Homo sapiens
QTPKYLVTQMGNDKSIKCEQNLGHDTMYWYKQDSKKFLKIMFSYNNKELIINETVPNRFSPKSPDKAHLNLHINSLELGDSAVYFCASNDRGRRTEAFFGQGTRLTVV

SEQ ID NO: 131 - protein, 132 aa, Homo sapiens
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASNDRGRRTEAFFGQGTRLTVV

SEQ ID NO: 132 - protein, 305 aa, synthetic construct
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASNDRGRRTEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 133 - protein, 6 aa, Homo sapiens
SSVSVY

SEQ ID NO: 134 - protein, 8 aa, Homo sapiens
YLSGSTLV

SEQ ID NO: 135 - protein, 14 aa, Homo sapiens
AVQFSSGGGADGLT

SEQ ID NO: 136 - protein, 115 aa, Homo sapiens
QSVTQLDSQVPVFEEAPVELRCNYSSSVSVYLFWYVQYPNQGLQLLLKYLSGSTLVKGINGFEAEFNKSQTSFHLRKPSVHISDTAEYFCAVQFSSGGGADGLTFGKGTHLIIQP

SEQ ID NO: 137 - protein, 135 aa, Homo sapiens
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVQFSSGGGADGLTFGKGTHLIIQP

SEQ ID NO: 138 - protein, 272 aa, synthetic construct
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVQFSSGGGADGLTFGKGTHLIIQPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 139 - protein, 5 aa, Homo sapiens
SGHDY

SEQ ID NO: 140 - protein, 6 aa, Homo sapiens
FNNNVP

SEQ ID NO: 141 - protein, 13 aa, Homo sapiens
ASSQNGLATDTQY

SEQ ID NO: 142 - protein, 113 aa, Homo sapiens
GVIQSPRHEVTEMGQEVTLRCKPISGHDYLFWYRQTMMRGLELLIYFNNNVPIDDSGMPEDRFSAKMPNASFSTLKIQPSEPRDSAVYFCASSQNGLATDTQYFGPGTRLTVL

SEQ ID NO: 143 - protein, 134 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSQNGLATDTQYFGPGTRLTVL

SEQ ID NO: 144 - protein, 307 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSQNGLATDTQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 145 - protein, 6 aa, Homo sapiens
TSGFYG

SEQ ID NO: 146 - protein, 6 aa, Homo sapiens
NALDGL

SEQ ID NO: 147 - protein, 11 aa, Homo sapiens
AVVSGGYNKLI

SEQ ID NO: 148 - protein, 109 aa, Homo sapiens
QSLEQPSEVTAVEGAIVQINCTYQTSGFYGLSWYQQHDGGAPTFLSYNALDGLEETGRFSSFLSRSDSYGYLLLQELQMKDSASYFCAVVSGGYNKLIFGAGTRLAVHP

SEQ ID NO: 149 - protein, 127 aa, Homo sapiens
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVVSGGYNKLIFGAGTRLAVHP

SEQ ID NO: 150 - protein, 264 aa, synthetic construct
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVVSGGYNKLIFGAGTRLAVHPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 151 - protein, 5 aa, Homo sapiens
LNHDA

SEQ ID NO: 152 - protein, 6 aa, Homo sapiens
SQIVND

SEQ ID NO: 153 - protein, 12 aa, Homo sapiens
ASRLDSGANVLT

SEQ ID NO: 154 - protein, 111 aa, Homo sapiens
GITQSPKYLFRKEGQNVTLSCEQNLNHDAMYWYRQDPGQGLRLIYYSQIVNDFQKGDIAEGYSVSREKKESFPLTVTSAQKNPTAFYLCASRLDSGANVLTFGAGSRLTVL

SEQ ID NO: 155 - protein, 132 aa, Homo sapiens
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASRLDSGANVLTFGAGSRLTVL

SEQ ID NO: 156 - protein, 305 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASRLDSGANVLTFGAGSRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 157 - protein, 7 aa, Homo sapiens
TRDTTYY

SEQ ID NO: 158 - protein, 8 aa, Homo sapiens
RNSFDEQN

SEQ ID NO: 159 - protein, 14 aa, Homo sapiens
ALSEERPGTASKLT

SEQ ID NO: 160 - protein, 116 aa, Homo sapiens
QKVTQAQTEISVVEKEDVTLDCVYETRDTTYYLFWYKQPPSGELVFLIRRNSFDEQNEISGRYSWNFQKSTSSFNFTITASQVVDSAVYFCALSEERPGTASKLTFGTGTRLQVTL

SEQ ID NO: 161 - protein, 137 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEERPGTASKLTFGTGTRLQVTL

SEQ ID NO: 162 - protein, 274 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEERPGTASKLTFGTGTRLQVTLNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 163 - protein, 5 aa, Homo sapiens
SGHTA

SEQ ID NO: 164 - protein, 6 aa, Homo sapiens
FQGTGA

SEQ ID NO: 165 - protein, 15 aa, Homo sapiens
ASSLTGTVTGTDTQY

SEQ ID NO: 166 - protein, 115 aa, Homo sapiens
GVSQTPSNKVTEKGKYVELRCDPISGHTALYWYRQSLGQGPEFLIYFQGTGAADDSGLPNDRFFAVRPEGSVSTLKIQRTERGDSAVYLCASSLTGTVTGTDTQYFGPGTRLTVL

SEQ ID NO: 167 - protein, 136 aa, Homo sapiens
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSLTGTVTGTDTQYFGPGTRLTVL

SEQ ID NO: 168 - protein, 309 aa, synthetic construct
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSLTGTVTGTDTQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 169 - protein, 6 aa, Homo sapiens
ATGYPS

SEQ ID NO: 170 - protein, 7 aa, Homo sapiens
ATKADDK

SEQ ID NO: 171 - protein, 11 aa, Homo sapiens
ALRTGANSKLT

SEQ ID NO: 172 - protein, 111 aa, Homo sapiens
NSVTQMEGPVTLSEEAFLTINCTYTATGYPSLFWYVQYPGEGLQLLLKATKADDKGSNKGFEATYRKETTSFHLEKGSVQVSDSAVYFCALRTGANSKLTFGKGITLSVRP

SEQ ID NO: 173 - protein, 131 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALRTGANSKLTFGKGITLSVRP

SEQ ID NO: 174 - protein, 268 aa, synthetic construct
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALRTGANSKLTFGKGITLSVRPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 175 - protein, 5 aa, Homo sapiens
SGHDN

SEQ ID NO: 176 - protein, 6 aa, Homo sapiens
FVKESK

SEQ ID NO: 177 - protein, 13 aa, Homo sapiens
ASSLSQSSNYGYT

SEQ ID NO: 178 - protein, 113 aa, Homo sapiens
GVTQFPSHSVIEKGQTVTLRCDPISGHDNLYWYRRVMGKEIKFLLHFVKESKQDESGMPNNRFLAERTGGTYSTLKVQPAELEDSGVYFCASSLSQSSNYGYTFGSGTRLTVV

SEQ ID NO: 179 - protein, 134 aa, Homo sapiens
MVSRLLSLVSLCLLGAKHIEAGVTQFPSHSVIEKGQTVTLRCDPISGHDNLYWYRRVMGKEIKFLLHFVKESKQDESGMPNNRFLAERTGGTYSTLKVQPAELEDSGVYFCASSLSQSSNYGYTFGSGTRLTVV

SEQ ID NO: 180 - protein, 307 aa, synthetic construct
MVSRLLSLVSLCLLGAKHIEAGVTQFPSHSVIEKGQTVTLRCDPISGHDNLYWYRRVMGKEIKFLLHFVKESKQDESGMPNNRFLAERTGGTYSTLKVQPAELEDSGVYFCASSLSQSSNYGYTFGSGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 181 - protein, 6 aa, Homo sapiens
NSASQS

SEQ ID NO: 182 - protein, 6 aa, Homo sapiens
VYSSGN

SEQ ID NO: 183 - protein, 12 aa, Homo sapiens
VVNTRGGYNKLI

SEQ ID NO: 184 - protein, 112 aa, Homo sapiens
QRKEVEQDPGPFNVPEGATVAFNCTYSNSASQSFFWYRQDCRKEPKLLMSVYSSGNEDGRFTAQLNRASQYISLLIRDSKLSDSATYLCVVNTRGGYNKLIFGAGTRLAVHP

SEQ ID NO: 185 - protein, 132 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNTRGGYNKLIFGAGTRLAVHP

SEQ ID NO: 186 - protein, 269 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNTRGGYNKLIFGAGTRLAVHPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 187 - protein, 5 aa, Homo sapiens
SGHTA

SEQ ID NO: 188 - protein, 6 aa, Homo sapiens
FQGNSA

SEQ ID NO: 189 - protein, 12 aa, Homo sapiens
ASSLAVGGTEAF

SEQ ID NO: 190 - protein, 111 aa, Homo sapiens
VSQSPSNKVTEKGKDVELRCDPISGHTALYWYRQRLGQGLEFLIYFQGNSAPDKSGLPSDRFSAERTGESVSTLTIQRTQQEDSAVYLCASSLAVGGTEAFFGQGTRLTVV

SEQ ID NO: 191 - protein, 133 aa, Homo sapiens
MGTRLLFWVAFCLLGAYHTGAGVSQSPSNKVTEKGKDVELRCDPISGHTALYWYRQRLGQGLEFLIYFQGNSAPDKSGLPSDRFSAERTGESVSTLTIQRTQQEDSAVYLCASSLAVGGTEAFFGQGTRLTVV

SEQ ID NO: 192 - protein, 306 aa, synthetic construct
MGTRLLFWVAFCLLGAYHTGAGVSQSPSNKVTEKGKDVELRCDPISGHTALYWYRQRLGQGLEFLIYFQGNSAPDKSGLPSDRFSAERTGESVSTLTIQRTQQEDSAVYLCASSLAVGGTEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 193 - protein, 5 aa, Homo sapiens
SIFNT

SEQ ID NO: 194 - protein, 7 aa, Homo sapiens
LYKAGEL

SEQ ID NO: 195 - protein, 11 aa, Homo sapiens
AGPGGATNKLI

SEQ ID NO: 196 - protein, 110 aa, Homo sapiens
QQLNQSPQSMFIQEGEDVSMNCTSSSIFNTWLWYKQDPGEGPVLLIALYKAGELTSNGRLTAQFGITRKDSFLNISASIPSDVGIYFCAGPGGATNKLIFGTGTLLAVQP

SEQ ID NO: 197 - protein, 129 aa, Homo sapiens
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGPGGATNKLIFGTGTLLAVQP

SEQ ID NO: 198 - protein, 266 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGPGGATNKLIFGTGTLLAVQPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 199 - protein, 5 aa, Homo sapiens
MNHNY

SEQ ID NO: 200 - protein, 6 aa, Homo sapiens
SVGAGI

SEQ ID NO: 201 - protein, 14 aa, Homo sapiens
ASRRSTSGLQETQY

SEQ ID NO: 202 - protein, 113 aa, Homo sapiens
GVTQTPKFRILKIGQSMTLQCTQDMNHNYMYWYRQDPGMGLKLIYYSVGAGITDKGEVPNGYNVSRSTTEDFPLRLELAAPSQTSVYFCASRRSTSGLQETQYFGPGTRLLVL

SEQ ID NO: 203 - protein, 134 aa, Homo sapiens
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASRRSTSGLQETQYFGPGTRLLVL

SEQ ID NO: 204 - protein, 307 aa, synthetic construct
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASRRSTSGLQETQYFGPGTRLLVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 205 - protein, 6 aa, Homo sapiens
VSGNPY

SEQ ID NO: 206 - protein, 8 aa, Homo sapiens
YITGDNLV

SEQ ID NO: 207 - protein, 12 aa, Homo sapiens
AVRDNTGGFKTI

SEQ ID NO: 208 - protein, 113 aa, Homo sapiens
QSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDNTGGFKTIFGAGTRLFVKA

SEQ ID NO: 209 - protein, 133 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDNTGGFKTIFGAGTRLFVKA

SEQ ID NO: 210 - protein, 270 aa, synthetic construct
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDNTGGFKTIFGAGTRLFVKANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 211 - protein, 5 aa, Homo sapiens
SGHAT

SEQ ID NO: 212 - protein, 6 aa, Homo sapiens
FQNNGV

SEQ ID NO: 213 - protein, 15 aa, Homo sapiens
ASSRRTSGSSYNEQF

SEQ ID NO: 214 - protein, 115 aa, Homo sapiens
GVAQSPRYKIIEKRQSVAFWCNPISGHATLYWYQQILGQGPKLLIQFQNNGVVDDSQLPKDRFSAERLKGVDSTLKIQPAKLEDSAVYLCASSRRTSGSSYNEQFFGPGTRLTVL

SEQ ID NO: 215 - protein, 136 aa, Homo sapiens
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSRRTSGSSYNEQFFGPGTRLTVL

SEQ ID NO: 216 - protein, 309 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSRRTSGSSYNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 217 - protein, 6 aa, Homo sapiens
VTNFRS

SEQ ID NO: 218 - protein, 7 aa, Homo sapiens
LTSSGIE

SEQ ID NO: 219 - protein, 13 aa, Homo sapiens
ALRGSGAGSYQLT

SEQ ID NO: 220 - protein, 113 aa, Homo sapiens
EDKVVQSPLSLVVHEGDTVTLNCSYEVTNFRSLLWYKQEKKAPTFLFMLTSSGIEKKSGRLSSILDKKELFSILNITATQTGDSAVYLCALRGSGAGSYQLTFGKGTKLSVIP

SEQ ID NO: 221 - protein, 134 aa, Homo sapiens
MMKCPQALLAIFWLLLSWVSSEDKVVQSPLSLVVHEGDTVTLNCSYEVTNFRSLLWYKQEKKAPTFLFMLTSSGIEKKSGRLSSILDKKELFSILNITATQTGDSAVYLCALRGSGAGSYQLTFGKGTKLSVIP

SEQ ID NO: 222 - protein, 271 aa, synthetic construct
MMKCPQALLAIFWLLLSWVSSEDKVVQSPLSLVVHEGDTVTLNCSYEVTNFRSLLWYKQEKKAPTFLFMLTSSGIEKKSGRLSSILDKKELFSILNITATQTGDSAVYLCALRGSGAGSYQLTFGKGTKLSVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 223 - protein, 5 aa, Homo sapiens
SGHVS

SEQ ID NO: 224 - protein, 6 aa, Homo sapiens
FQNEAQ

SEQ ID NO: 225 - protein, 13 aa, Homo sapiens
ASSLEGGGPNEQF

SEQ ID NO: 226 - protein, 113 aa, Homo sapiens
GVSQSPRYKVAKRGQDVALRCDPISGHVSLFWYQQALGQGPEFLTYFQNEAQLDKSGLPSDRFFAERPEGSVSTLKIQRTQQEDSAVYLCASSLEGGGPNEQFFGPGTRLTVL

SEQ ID NO: 227 - protein, 134 aa, Homo sapiens
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLEGGGPNEQFFGPGTRLTVL

SEQ ID NO: 228 - protein, 307 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLEGGGPNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 229 - protein, 6 aa, Homo sapiens
ATGYPS

SEQ ID NO: 230 - protein, 7 aa, Homo sapiens
ATKADDK

SEQ ID NO: 231 - protein, 11 aa, Homo sapiens
ALSTGSARQLT

SEQ ID NO: 232 - protein, 111 aa, Homo sapiens
NSVTQMEGPVTLSEEAFLTINCTYTATGYPSLFWYVQYPGEGLQLLLKATKADDKGSNKGFEATYRKETTSFHLEKGSVQVSDSAVYFCALSTGSARQLTFGSGTQLTVLP

SEQ ID NO: 233 - protein, 131 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSTGSARQLTFGSGTQLTVLP

SEQ ID NO: 234 - protein, 268 aa, synthetic construct
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSTGSARQLTFGSGTQLTVLPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 235 - protein, 5 aa, Homo sapiens
LNHDA

SEQ ID NO: 236 - protein, 6 aa, Homo sapiens
SQIVND

SEQ ID NO: 237 - protein, 15 aa, Homo sapiens
ASSISGTVSGANVLT

SEQ ID NO: 238 - protein, 114 aa, Homo sapiens
GITQSPKYLFRKEGQNVTLSCEQNLNHDAMYWYRQDPGQGLRLIYYSQIVNDFQKGDIAEGYSVSREKKESFPLTVTSAQKNPTAFYLCASSISGTVSGANVLTFGAGSRLTVL

SEQ ID NO: 239 - protein, 135 aa, Homo sapiens
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSISGTVSGANVLTFGAGSRLTVL

SEQ ID NO: 240 - protein, 308 aa, synthetic construct
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSISGTVSGANVLTFGAGSRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 241 - protein, 6 aa, Homo sapiens
TSGFYG

SEQ ID NO: 242 - protein, 6 aa, Homo sapiens
NALDGL

SEQ ID NO: 243 - protein, 11 aa, Homo sapiens
AVLSGGYNKLI

SEQ ID NO: 244 - protein, 109 aa, Homo sapiens
QSLEQPSEVTAVEGAIVQINCTYQTSGFYGLSWYQQHDGGAPTFLSYNALDGLEETGRFSSFLSRSDSYGYLLLQELQMKDSASYFCAVLSGGYNKLIFGAGTRLAVHP

SEQ ID NO: 245 - protein, 127 aa, Homo sapiens
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVLSGGYNKLIFGAGTRLAVHP

SEQ ID NO: 246 - protein, 264 aa, synthetic construct
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVLSGGYNKLIFGAGTRLAVHPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 247 - protein, 5 aa, Homo sapiens
LNHDA

SEQ ID NO: 248 - protein, 6 aa, Homo sapiens
SQIVND

SEQ ID NO: 249 - protein, 12 aa, Homo sapiens
ASRKDSGENGYT

SEQ ID NO: 250 - protein, 111 aa, Homo sapiens
GITQSPKYLFRKEGQNVTLSCEQNLNHDAMYWYRQDPGQGLRLIYYSQIVNDFQKGDIAEGYSVSREKKESFPLTVTSAQKNPTAFYLCASRKDSGENGYTFGSGTRLTVV

SEQ ID NO: 251 - protein, 132 aa, Homo sapiens
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASRKDSGENGYTFGSGTRLTVV

SEQ ID NO: 252 - protein, 305 aa, synthetic construct
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASRKDSGENGYTFGSGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 253 - protein, 5 aa, Homo sapiens
VGISA

SEQ ID NO: 254 - protein, 5 aa, Homo sapiens
LSSGK

SEQ ID NO: 255 - protein, 12 aa, Homo sapiens
AVWEETSGSRLT

SEQ ID NO: 256 - protein, 111 aa, Homo sapiens
AKNEVEQSPQNLTAQEGEFITINCSYSVGISALHWLQQHPGGGIVSLFMLSSGKKKHGRLIATINIQEKHSSLHITASHPRDSAVYICAVWEETSGSRLTFGEGTQLTVNP

SEQ ID NO: 257 - protein, 132 aa, Homo sapiens
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVWEETSGSRLTFGEGTQLTVNP

SEQ ID NO: 258 - protein, 269 aa, synthetic construct
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVWEETSGSRLTFGEGTQLTVNPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 259 - protein, 5 aa, Homo sapiens
SNHLY

SEQ ID NO: 260 - protein, 6 aa, Homo sapiens
FYNNEI

SEQ ID NO: 261 - protein, 13 aa, Homo sapiens
ASTRDTWSTDTQY

SEQ ID NO: 262 - protein, 115 aa, Homo sapiens
EPEVTQTPSHQVTQMGQEVILCCVPISNHLYFYWYRQILGQKVEFLVSFYNNEISEKSEIFDDQFSVERPDGSNFTLKIRSTKLEDSAMYFCASTRDTWSTDTQYFGPGTRLTVL

SEQ ID NO: 263 - protein, 134 aa, Homo sapiens
MDTWLVCWAIFSLLKAGLTEPEVTQTPSHQVTQMGQEVILCCVPISNHLYFYWYRQILGQKVEFLVSFYNNEISEKSEIFDDQFSVERPDGSNFTLKIRSTKLEDSAMYFCASTRDTWSTDTQYFGPGTRLTVL

SEQ ID NO: 264 - protein, 307 aa, synthetic construct
MDTWLVCWAIFSLLKAGLTEPEVTQTPSHQVTQMGQEVILCCVPISNHLYFYWYRQILGQKVEFLVSFYNNEISEKSEIFDDQFSVERPDGSNFTLKIRSTKLEDSAMYFCASTRDTWSTDTQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 265 - protein, 6 aa, Homo sapiens
DRGSQS

SEQ ID NO: 266 - protein, 6 aa, Homo sapiens
IYSNGD

SEQ ID NO: 267 - protein, 13 aa, Homo sapiens
AVKASSGSARQLT

SEQ ID NO: 268 - protein, 114 aa, Homo sapiens
QQKEVEQNSGPLSVPEGAIASLNCTYSDRGSQSFFWYRQYSGKSPELIMFIYSNGDKEDGRFTAQLNKASQYVSLLIRDSQPSDSATYLCAVKASSGSARQLTFGSGTQLTVLP

SEQ ID NO: 269 - protein, 134 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKASSGSARQLTFGSGTQLTVLP

SEQ ID NO: 270 - protein, 271 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKASSGSARQLTFGSGTQLTVLPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 271 - protein, 5 aa, Homo sapiens
MNHEY

SEQ ID NO: 272 - protein, 6 aa, Homo sapiens
SVGEGT

SEQ ID NO: 273 - protein, 13 aa, Homo sapiens
ASSYKLAGDNEQF

SEQ ID NO: 274 - protein, 112 aa, Homo sapiens
GVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYKLAGDNEQFFGPGTRLTVL

SEQ ID NO: 275 - protein, 133 aa, Homo sapiens
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYKLAGDNEQFFGPGTRLTVL

SEQ ID NO: 276 - protein, 306 aa, synthetic construct
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYKLAGDNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 277 - protein, 6 aa, Homo sapiens
YGATPY

SEQ ID NO: 278 - protein, 8 aa, Homo sapiens
YFSGDTLV

SEQ ID NO: 279 - protein, 9 aa, Homo sapiens
AVGRNTPLV

SEQ ID NO: 280 - protein, 110 aa, Homo sapiens
QSVTQPDIHITVSEGASLELRCNYSYGATPYLFWYVQSPGQGLQLLLKYFSGDTLVQGIKGFEAEFKRSQSSFNLRKPSVHWSDAAEYFCAVGRNTPLVFGKGTRLSVIA

SEQ ID NO: 281 - protein, 130 aa, Homo sapiens
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGRNTPLVFGKGTRLSVIA

SEQ ID NO: 282 - protein, 267 aa, synthetic construct
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGRNTPLVFGKGTRLSVIANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 283 - protein, 5 aa, Homo sapiens
SGHTA

SEQ ID NO: 284 - protein, 6 aa, Homo sapiens
FQGNSA

SEQ ID NO: 285 - protein, 16 aa, Homo sapiens
ASSSGGAFDRSGNTIY

SEQ ID NO: 286 - protein, 116 aa, Homo sapiens
GVSQSPSNKVTEKGKDVELRCDPISGHTALYWYRQSLGQGLEFLIYFQGNSAPDKSGLPSDRFSAERTGGSVSTLTIQRTQQEDSAVYLCASSSGGAFDRSGNTIYFGEGSWLTVV

SEQ ID NO: 287 - protein, 137 aa, Homo sapiens
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSSGGAFDRSGNTIYFGEGSWLTVV

SEQ ID NO: 288 - protein, 310 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSSGGAFDRSGNTIYFGEGSWLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 289 - protein, 6 aa, Homo sapiens
YGATPY

SEQ ID NO: 290 - protein, 8 aa, Homo sapiens
YFSGDTLV

SEQ ID NO: 291 - protein, 10 aa, Homo sapiens
AGRGGGFKTI

SEQ ID NO: 292 - protein, 111 aa, Homo sapiens
QSVTQPDIHITVSEGASLELRCNYSYGATPYLFWYVQSPGQGLQLLLKYFSGDTLVQGIKGFEAEFKRSQSSFNLRKPSVHWSDAAEYFCAGRGGGFKTIFGAGTRLFVKA

SEQ ID NO: 293 - protein, 131 aa, Homo sapiens
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAGRGGGFKTIFGAGTRLFVKA

SEQ ID NO: 294 - protein, 268 aa, synthetic construct
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAGRGGGFKTIFGAGTRLFVKANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 295 - protein, 5 aa, Homo sapiens
LGHDT

SEQ ID NO: 296 - protein, 6 aa, Homo sapiens
YNNKEL

SEQ ID NO: 297 - protein, 14 aa, Homo sapiens
ASSSRLAGAQETQY

SEQ ID NO: 298 - protein, 110 aa, Homo sapiens
QTPKYLVTQMGNDKSIKCEQNLGHDTMYWYKQDSKKFLKIMFSYNNKELIINETVPNRFSPKSPDKAHLNLHINSLELGDSAVYFCASSSRLAGAQETQYFGPGTRLLVL

SEQ ID NO: 299 - protein, 134 aa, Homo sapiens
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSSRLAGAQETQYFGPGTRLLVL

SEQ ID NO: 300 - protein, 307 aa, synthetic construct
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSSRLAGAQETQYFGPGTRLLVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 301 - protein, 365 aa, Homo sapiens
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 302 - protein, 362 aa, Homo sapiens [sequence omitted]

SEQ ID NO: 303 - protein, 260 aa, Homo sapiens
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 304 - protein, 260 aa, Homo sapiens [sequence omitted]

SEQ ID NO: 305 - protein, 258 aa, Homo sapiens [sequence omitted]

SEQ ID NO: 306 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 307 - protein, 266 aa, Homo sapiens
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 308 - protein, 266 aa, Homo sapiens [sequence omitted]

SEQ ID NO: 309 - protein, 266 aa, Homo sapiens
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 310 - protein, 25 aa, synthetic construct
RRRYRKETLQALYMPVLPVTATEIR

SEQ ID NO: 311 - protein, 25 aa, synthetic construct
TSREKEEGSDQHEASHLPLHPIMHN

SEQ ID NO: 312 - protein, 25 aa, synthetic construct
VLYCYEVQLESKLRRKGLGKFLIQI

SEQ ID NO: 313 - protein, 25 aa, synthetic construct
TTNLGGPECCLRISLMPLRLNVDQD

SEQ ID NO: 314 - protein, 25 aa, synthetic construct
SHMWIYVFMGNMRRGIGETPIVPLG

SEQ ID NO: 315 - protein, 25 aa, synthetic construct
SQSPNACRIHGHPYVNKVAGNFHIT

SEQ ID NO: 316 - protein, 25 aa, synthetic construct
PSARRSYLCTLPLALLSREILMADS

SEQ ID NO: 317 - protein, 25 aa, synthetic construct
SSFLLSYLCTLPLALLSREILMADS

SEQ ID NO: 318 - protein, 25 aa, synthetic construct
EEKAHSEQIHKLWRELVASQEKVAT

SEQ ID NO: 319 - protein, 25 aa, synthetic construct
LIALASWSWALCCISLLPLIVTFHL

SEQ ID NO: 320 - protein, 25 aa, synthetic construct
VNDVDLPPWAKKTEDFVRINRMALE

SEQ ID NO: 321 - protein, 25 aa, synthetic construct
HVGSRGGPSERSRARNAHFRRTASF

SEQ ID NO: 322 - protein, 25 aa, synthetic construct
FENLVEETSHFVCTTFVSRHKKFDE

SEQ ID NO: 323 - protein, 25 aa, synthetic construct
EMSFYWSLLFRLVFDVKRKDFLAHI

SEQ ID NO: 324 - protein, 25 aa, synthetic construct
GEKGDKGDVSNDMLLAGAKGDQGPP

SEQ ID NO: 325 - protein, 25 aa, synthetic construct
DEDLDLIPPKPMVSSTCSCCWCCLG

SEQ ID NO: 326 - protein, 25 aa, synthetic construct
ELSTPAATILCLLGAQSNLSPLQLA

SEQ ID NO: 327 - protein, 25 aa, synthetic construct
LFWMEFKGHCYRLFPLNKTWAEADL

SEQ ID NO: 328 - protein, 25 aa, synthetic construct
RSCIGRYNDVFFGVLKKILDSLISD

SEQ ID NO: 329 - protein, 25 aa, synthetic construct
APLAIATARGYTGCARHLIRQGAEL

SEQ ID NO: 330 - protein, 25 aa, synthetic construct
SESSRVDFGSSEHLGSWQEKEEDAR

SEQ ID NO: 331 - protein, 25 aa, synthetic construct
RHIKGPKDVERSPIFSKLCLNIRLA

SEQ ID NO: 332 - protein, 25 aa, synthetic construct
SGNLLGRNSFEVCVCACPGRDRRTE

SEQ ID NO: 333 - protein, 25 aa, synthetic construct
GSWSSAPTSRASLPPPQPQPPPPPA

SEQ ID NO: 334 - protein, 25 aa, synthetic construct
HQKTHTGERPYQWDKCGKGFSHNSY

SEQ ID NO: 335 - protein, 25 aa, synthetic construct
HSQHYQIPDDFVPDVRLIFKNCERF

SEQ ID NO: 336 - protein, 25 aa, synthetic construct
LFWRMPIPELDLLELEGLGLSDTAT

SEQ ID NO: 337 - protein, 25 aa, synthetic construct
QNQCCQSKGNQCYPPKQNQCCQPKG

SEQ ID NO: 338 - protein, 25 aa, synthetic construct
ITNNPRLSFIHPHAFHHLPQMETLM

SEQ ID NO: 339 - protein, 25 aa, synthetic construct
RGLQESHGRDGTHFCHLLDLPDKDY

SEQ ID NO: 340 - protein, 25 aa, synthetic construct
VLNYFQPFLGRIKIMGSAKRIERVY

SEQ ID NO: 341 - protein, 25 aa, synthetic construct
TSAFERLLVICCAVMLIFPVSVIIL

SEQ ID NO: 342 - protein, 25 aa, synthetic construct
ILHLIHRMNSAASHRKALATCSSHM

SEQ ID NO: 343 - protein, 25 aa, synthetic construct
LAGNAVIFLTIQRFFLIFGRGDDFS

SEQ ID NO: 344 - protein, 25 aa, synthetic construct
LTKAALPAHSFVGSLSLKMNRCLKY

SEQ ID NO: 345 - protein, 25 aa, synthetic construct
EMFPAFTDSRECQLLKKLLEAHEEQ

SEQ ID NO: 346 - protein, 25 aa, synthetic construct
QRSQLQGAMQQFKQRFIYGNQDLFA

SEQ ID NO: 347 - protein, 25 aa, synthetic construct
CPRCRRLLHKPVMLPCGLTVCKRCV

SEQ ID NO: 348 - protein, 25 aa, synthetic construct
TRNDGKLYDAYVFYPRNYKSSTDGA

SEQ ID NO: 349 - protein, 25 aa, synthetic construct
ASAPECRCSLLACEGLLCGQPEVGA

SEQ ID NO: 350 - protein, 25 aa, synthetic construct
IEGAWRADGKGLIIWDTFSHTPLRV

SEQ ID NO: 351 - protein, 25 aa, synthetic construct
NIIVASFDGRGSVYQGDKIMHAINR

SEQ ID NO: 352 - protein, 25 aa, synthetic construct
EDIERQRKLLAKCKPPTANNSQAPS

SEQ ID NO: 353 - protein, 25 aa, synthetic construct
QMFSVSEISTVAHEITDSVLNILHK

SEQ ID NO: 354 - protein, 25 aa, synthetic construct
KQLIREKREMTKNIHKMEETVRQLM

SEQ ID NO: 355 - protein, 25 aa, synthetic construct
AGLLVDSSEMIFNFDGRQGAKVPDG

SEQ ID NO: 356 - protein, 15 aa, synthetic construct
SAQRWIEAMEDATVL

SEQ ID NO: 357 - protein, 25 aa, synthetic construct
MESERDRQHFDKVVMKAYIEDLKER

SEQ ID NO: 358 - protein, 25 aa, synthetic construct
AQLELQMTALRYRKKFTEYSARLDS

SEQ ID NO: 359 - protein, 25 aa, synthetic construct
FQEHFGIYTGKIFIDFPTAAQFDNL

SEQ ID NO: 360 - protein, 25 aa, synthetic construct
PAIVGSCATVLAFLVLSLILCNQCR

SEQ ID NO: 361 - protein, 15 aa, synthetic construct
KGIPEGGGMRSTMKT

SEQ ID NO: 362 - protein, 25 aa, synthetic construct
LSRTFLRVNAFPGVLACESVDVDLC

SEQ ID NO: 363 - protein, 14 aa, synthetic construct
VMEELKETTNSSKI

SEQ ID NO: 364 - protein, 25 aa, synthetic construct
DQWTSIASMQERQSTLGAAVLNDLL

SEQ ID NO: 365 - protein, 25 aa, synthetic construct
QLTIKTLSVPVKKDAQLGTVIALIS

SEQ ID NO: 366 - protein, 25 aa, synthetic construct
ESVSAYELVVTAQDRGSPSLWATAR

SEQ ID NO: 367 - protein, 25 aa, synthetic construct
VNLAVLKLSEQGILDKLKSKWWYDK

SEQ ID NO: 368 - protein, 19 aa, synthetic construct
GVVVYSGGRYDLMPSSSAY

SEQ ID NO: 369 - protein, 25 aa, synthetic construct
AANMPEDYPDQFHDVMDFIQATIKR

SEQ ID NO: 370 - protein, 25 aa, synthetic construct
DSNMPEDYPDQFHDVMDFIQATIKR

SEQ ID NO: 371 - protein, 25 aa, synthetic construct
QHGPHSAVARISWAQYNALTLTFQR

SEQ ID NO: 372 - protein, 25 aa, synthetic construct
YFFLTNLSILDLYYTTTTVPHMLVN

SEQ ID NO: 373 - protein, 25 aa, synthetic construct
GSSRTVWCQRSDCINVTGGCLVMSQ

SEQ ID NO: 374 - protein, 25 aa, synthetic construct
KHTIIISWGGVNMPKSPFRVNVGEG

SEQ ID NO: 375 - protein, 25 aa, synthetic construct
ERDLVCGSKSDSGVVVTGGDSGESQ

SEQ ID NO: 376 - protein, 25 aa, synthetic construct
AKETGSHIEGVTSARETERTMSATS

SEQ ID NO: 377 - protein, 25 aa, synthetic construct
YALRNFVEEKLGVKYVERTRLDLVK

SEQ ID NO: 378 - protein, 25 aa, synthetic construct
ILMTKLRASTTSKTIQYRKAVKATL

SEQ ID NO: 379 - protein, 25 aa, synthetic construct
PSPQAIWSITCKEDIFVSEPSPDLE

SEQ ID NO: 380 - protein, 25 aa, synthetic construct
SRNPDNYVRETKFDLQRVPRNYDPA

SEQ ID NO: 381 - protein, 25 aa, synthetic construct
LGVLEQFIPLQLHEYGCPRATTVRR

SEQ ID NO: 382 - protein, 25 aa, synthetic construct
PHYFPQSPPQGEGSLSPHYFPQSPQ

SEQ ID NO: 383 - protein, 25 aa, synthetic construct
LGYIKKVIDTRLFPHLKDISPSENV

SEQ ID NO: 384 - protein, 25 aa, synthetic construct
NPMPIRSKSPEHMRNIIALSIEATA

SEQ ID NO: 385 - protein, 10 aa, synthetic construct
RIHGHPYVNK

SEQ ID NO: 386 - protein, 25 aa, synthetic construct
ALLGLASVIKNTSLYFADKLHQALQ

SEQ ID NO: 387 - protein, 17 aa, synthetic construct
GSARPSFGDQEHIAVLC

SEQ ID NO: 388 - protein, 25 aa, synthetic construct
LIPGNVSSLFTIESTTGIIYLTLPL

SEQ ID NO: 389 - protein, 25 aa, synthetic construct
LASAPLPPSTLASVSASASVSASVP

SEQ ID NO: 390 - protein, 25 aa, synthetic construct
AEKEPEFLNISIHDSRLFFQLQSGN

SEQ ID NO: 391 - protein, 25 aa, synthetic construct
PEHEMENYSMYCMSCRTPVCYLCLE

SEQ ID NO: 392 - protein, 25 aa, synthetic construct
HKNPVPQCPPRLRVQFLSHVLWSRM

SEQ ID NO: 393 - protein, 25 aa, synthetic construct
SCRLVQALPTVICSAGCIPSNIVWD

SEQ ID NO: 394 - protein, 25 aa, synthetic construct
DKDPGGMLRRNLWNQSYRAAMKGLG

SEQ ID NO: 395 - protein, 25 aa, synthetic construct
HCVWLILARPESHIHLAFNDIDVEP

SEQ ID NO: 396 - protein, 25 aa, synthetic construct
SLPPPSPAHLAAFSVAVIELAKEHG

SEQ ID NO: 397 - protein, 25 aa, synthetic construct
FCIVGTLYLYRCLTMYVTTLPVPGM

SEQ ID NO: 398 - protein, 25 aa, synthetic construct
HKEWIVIGQVDMAALVEKHLFTVHD

SEQ ID NO: 399 - protein, 25 aa, synthetic construct
TITQIPKKSGVEYEDQQEVILVRTD

SEQ ID NO: 400 - protein, 25 aa, synthetic construct
VSLVYSMPSRNLYLRLEGLQEKDSG

SEQ ID NO: 401 - protein, 25 aa, synthetic construct
WLESGIPNLGVWAKRIHTTAEKYRE

SEQ ID NO: 402 - protein, 25 aa, synthetic construct
ETVIGRGLKTTDRLAVDWVARNLYW

SEQ ID NO: 403 - protein, 13 aa, synthetic construct
VQAAAETLSEVAQ

SEQ ID NO: 404 - protein, 25 aa, synthetic construct
VRGYQVHVLTFTIHMLLQGLTNKLQ

SEQ ID NO: 405 - protein, 25 aa, synthetic construct
ETCLLDILDTAGHEEYSAMRDQYMR

SEQ ID NO: 406 - protein, 25 aa, synthetic construct
FSDSLSTSIKYSQPGETDGTRLLLI

SEQ ID NO: 407 - protein, 25 aa, synthetic construct
KVERYVESESDLLLQNCESLTSDSA

SEQ ID NO: 408 - protein, 25 aa, synthetic construct
TKDLTEALDEDANPTKSALSAVASL

SEQ ID NO: 409 - protein, 25 aa, synthetic construct
LNSKSESLPVSDNTAISETPLVSQF

SEQ ID NO: 410 - protein, 25 aa, synthetic construct
PPAPQPSSRFNDVPAADKEPSVVQF

SEQ ID NO: 411 - protein, 25 aa, synthetic construct
VYLYTVVAFNFFCKFYNKSEDDDEP

SEQ ID NO: 412 - protein, 25 aa, synthetic construct
SIFSQEYQKHIKTTHAKHHTSEAIE

SEQ ID NO: 413 - protein, 25 aa, synthetic construct
NRVKNISDADVHTAMDNYECNLYTF

SEQ ID NO: 414 - protein, 25 aa, synthetic construct
PKASPSLTRFCSQSSLPTGSSVMLW

SEQ ID NO: 415 - protein, 25 aa, synthetic construct
SFKRPCPLARYNHTSYFYPTFSESS

SEQ ID NO: 416 - protein, 24 aa, synthetic construct
QFWKKALPQKIQKLEEPEERHTEL

SEQ ID NO: 417 - protein, 25 aa, synthetic construct
DILFSKTPQEDYMEAAVKQSLQVHL

SEQ ID NO: 418 - protein, 25 aa, synthetic construct
ERRIHQESELHSCLSRLIAAERERE

SEQ ID NO: 419 - protein, 25 aa, synthetic construct
TCVRNTVPCRKFIKINNSTNEGMNV

SEQ ID NO: 420 - protein, 25 aa, synthetic construct
GTRALLEFLRVHSDVRAVGAEPHFF

SEQ ID NO: 421 - protein, 25 aa, synthetic construct
VFLINCIVFVLRCRHKRIPPEGQTS

SEQ ID NO: 422 - protein, 25 aa, synthetic construct
YMCNSSCMGGMNQRPILTIITLEDS

SEQ ID NO: 423 - protein, 25 aa, synthetic construct
AATDPAKPLLSYEGSCPPSPPSVIS

SEQ ID NO: 424 - protein, 25 aa, synthetic construct
CKECGKSFSSLGILQRHMAVQRGDG

SEQ ID NO: 425 - protein, 25 aa, synthetic construct
NGYYPAHGLARPHGPGSRKGLHEPY

SEQ ID NO: 426 - protein, 25 aa, synthetic construct
ALELHNCMAKLLSHPLQRPCQSHAS

SEQ ID NO: 427 - protein, 25 aa, synthetic construct
ESRRVHLGSVEKYGSTFSRELVLVT

SEQ ID NO: 428 - protein, 25 aa, synthetic construct
QPDLPPQAKKAGCGLFGGWSEHRED

SEQ ID NO: 429 - protein, 25 aa, synthetic construct
FLYEFCVGLSLGTHSAIVTAYGIGI

SEQ ID NO: 430 - protein, 25 aa, synthetic construct
KSFNRRESLKRHAKTHSADLLRLPC

SEQ ID NO: 431 - protein, 25 aa, synthetic construct
KNPLLHLKAAVKGKKRNKKKKTIGS

SEQ ID NO: 432 - protein, 25 aa, synthetic construct
QGRNYIVEDGDINFFKFNTPQQPKK

SEQ ID NO: 433 - protein, 25 aa, synthetic construct
SQTTLEEVFINFSKDQKSYETADTS

SEQ ID NO: 434 - protein, 25 aa, synthetic construct
RFPPQCYATQYQNYSLSSAHKVSGM

SEQ ID NO: 435 - protein, 25 aa, synthetic construct
LYLPLIQERLTDILRVGQTSIVAAQ

SEQ ID NO: 436 - protein, 17 aa, synthetic construct
ACLPTTVGHAGKKQRRD

SEQ ID NO: 437 - protein, 25 aa, synthetic construct
PSEDLQEQIYLNWTLLTTISTQRVL

SEQ ID NO: 438 - protein, 25 aa, synthetic construct
CASDIRSSSLTLTWYGSSYDGGSAV

SEQ ID NO: 439 - protein, 25 aa, synthetic construct
ISEENTKLRRQSSFSVAGKDKSPKK

SEQ ID NO: 440 - protein, 19 aa, synthetic construct
MARKKFCGLEISLIVLFVI

SEQ ID NO: 441 - protein, 25 aa, synthetic construct
GNKICQFKLVLLEESAVGKSSLVLR

SEQ ID NO: 442 - protein, 25 aa, synthetic construct
SAALVLLLLFMMMVFFAKKLYLLKT

SEQ ID NO: 443 - protein, 25 aa, synthetic construct
RPLKSAEEAYEELMRKAELLQRQQG

SEQ ID NO: 444 - protein, 25 aa, synthetic construct
SQKDYRKDASIGMKVVCWFVHNNGE

SEQ ID NO: 445 - protein, 25 aa, synthetic construct
LYPAIMTNGLCIQLLILSYVGGLLH

SEQ ID NO: 446 - protein, 25 aa, synthetic construct
NSKSRLNLFTQSISRVFESHSFNSI

SEQ ID NO: 447 - protein, 25 aa, synthetic construct
VETGNCIHTLTGQQSLTSGMELKDN

SEQ ID NO: 448 - protein, 25 aa, synthetic construct
PPPEPGKPKGKRWRRRGWRRVTEGK

SEQ ID NO: 449 - protein, 25 aa, synthetic construct
SRFDTDDELSYRHDSVYSCVTLPYF

SEQ ID NO: 450 - protein, 25 aa, synthetic construct
ERMHFIHRDLAASNCLVSVKDYTSP

SEQ ID NO: 451 - protein, 25 aa, synthetic construct
AHLTCEGDKKEEASGSNKSGKVHAC

SEQ ID NO: 452 - protein, 25 aa, synthetic construct
ATLRAPRRLSWAVLLLLAALLPVAS

SEQ ID NO: 453 - protein, 25 aa, synthetic construct
GSPLSGGAEEASEGTGYGGEGSGPG

SEQ ID NO: 454 - protein, 25 aa, synthetic construct
FIDKLALRAGNEEEDGEAADTVGCC

SEQ ID NO: 455 - protein, 25 aa, synthetic construct
KCTVCNEATPIKTPPTGKKYVRCPC

SEQ ID NO: 456 - protein, 25 aa, synthetic construct
GDQGLPGDRGPPEPPGIRGPPGPPG

SEQ ID NO: 457 - protein, 23 aa, synthetic construct
SARNPNLELPMLFSYKHTDSGYS

SEQ ID NO: 458 - protein, 25 aa, synthetic construct
LIPGNVSSLFTIESTTGLYSPEVEI

SEQ ID NO: 459 - protein, 25 aa, synthetic construct
STSAPVPASPLASVSASASVSASVP

SEQ ID NO: 460 - protein, 25 aa, synthetic construct
NHVYRRSLGKYTGFKMFSDEILLSL

SEQ ID NO: 461 - protein, 25 aa, synthetic construct
QENLENGSARKQEAESVLQNGLRRE

SEQ ID NO: 462 - protein, 25 aa, synthetic construct
KISCFNNFSLSTSGSSSWPTHSQMD

SEQ ID NO: 463 - protein, 25 aa, synthetic construct
ARCRSESDMENEQNSNTSKQRYSGK

SEQ ID NO: 464 - protein, 25 aa, synthetic construct
LAQGGRRDARSFIEAAGTTNFPANE

SEQ ID NO: 465 - protein, 25 aa, synthetic construct
VLKRDPTLYRDVTKNALLRWRVFIY

SEQ ID NO: 466 - protein, 25 aa, synthetic construct
GGRGPAATTSSTAVAVGAEHGEDSL

SEQ ID NO: 467 - protein, 25 aa, synthetic construct
LFLVRESTNYPGYYTLCVSCDGKVE

SEQ ID NO: 468 - protein, 25 aa, synthetic construct
RCQKMCPSTCGKQACTENNECCHPE

SEQ ID NO: 469 - protein, 25 aa, synthetic construct
KRAIANGQRALPWDGPLDEPGLGVY

SEQ ID NO: 470 - protein, 25 aa, synthetic construct
QIMKVEGHVFPEIKESDAMFAAERA

SEQ ID NO: 471 - protein, 25 aa, synthetic construct
VDGGASPQFDLDEFISRALKLCTKP

SEQ ID NO: 472 - protein, 25 aa, synthetic construct
VEAAAATAVVVVKLSSPSTNEEFCC

SEQ ID NO: 473 - protein, 25 aa, synthetic construct
VFDAMFNGGMATKSTEIELPDVEPA

SEQ ID NO: 474 - protein, 25 aa, synthetic construct
AFREDGSLRAHNAHGREKMYDFTQC

SEQ ID NO: 475 - protein, 25 aa, synthetic construct
ESELQAYIAQCQHSPTSGKFRRGSG

SEQ ID NO: 476 - protein, 25 aa, synthetic construct
NPVEGERYLDFLSFAVFCNGESVEE

SEQ ID NO: 477 - protein, 25 aa, synthetic construct
VGSVDGFMLTPISMSFPFCRSHEIQ

SEQ ID NO: 478 - protein, 25 aa, synthetic construct
KLQYQRAALQAKHSQDLEQAKAYLR

SEQ ID NO: 479 - protein, 25 aa, synthetic construct
AHPEEISSNSQVLSRSPKKRPEPVP

SEQ ID NO: 480 - protein, 25 aa, synthetic construct
LVVALLALLCHLCKKQGAARAKMEY

SEQ ID NO: 481 - protein, 25 aa, synthetic construct
NPIHARGKVPPHARHYPLMFCHVAG

SEQ ID NO: 482 - protein, 25 aa, synthetic construct
GAAQLKENNGKGFAESGSAAGTTTS

SEQ ID NO: 483 - protein, 25 aa, synthetic construct
SVSGILVCQAGCILEELSRYVEERD

SEQ ID NO: 484 - protein, 25 aa, synthetic construct
GPSLVPGKTPTVCAALICLTLVLVA

SEQ ID NO: 485 - protein, 25 aa, synthetic construct
ALEYFMKQMNDARHGGWTTKMDWIF

SEQ ID NO: 486 - protein, 24 aa, synthetic construct
GAIGGLLEQFARVVGLRSISSNAL

SEQ ID NO: 487 - protein, 25 aa, synthetic construct
TLDNRVADQLWVQDTYFLNDKKSFV

SEQ ID NO: 488 - protein, 25 aa, synthetic construct
RERQLFSIDASTWEVRVIGGLDYEE

SEQ ID NO: 489 - protein, 25 aa, synthetic construct
LDVTWQGVEDNKKNCIVYSKEDIFP

SEQ ID NO: 490 - protein, 25 aa, synthetic construct
LDVTWQGVEDNKKNCIVYSKGGQLW

SEQ ID NO: 491 - protein, 25 aa, synthetic construct
ANIYPRCSVNGKLTSTQVLPVGWSP

SEQ ID NO: 492 - protein, 17 aa, synthetic construct
GEPWPGPASPSGDAAWR

SEQ ID NO: 493 - protein, 25 aa, synthetic construct
SGATVTLWMSLVITSMVAVIVYRLS

SEQ ID NO: 494 - protein, 25 aa, synthetic construct
RRVMLGASRDLLWSLYDCDLYFLEN

SEQ ID NO: 495 - protein, 25 aa, synthetic construct
KQKLVEKIMDQYHVLEPVPLPRTKK

SEQ ID NO: 496 - protein, 25 aa, synthetic construct
TTDIISVANEDGQRVAAILNHAHYL

SEQ ID NO: 497 - protein, 24 aa, synthetic construct
MTEYKLVVVGACGVGKSALTIQLI

SEQ ID NO: 498 - protein, 25 aa, synthetic construct
RRQSQSRSPKRDSTRESRRSESLSP

SEQ ID NO: 499 - protein, 25 aa, synthetic construct
AGGGIHFTVERTCQATEEELFRSLQ

SEQ ID NO: 500 - protein, 25 aa, synthetic construct
RGVNRGMVSTLLCETPSFGVYFLTY

SEQ ID NO: 501 - protein, 25 aa, synthetic construct
SLELDYHTSSCFQGTMVKADCPVPI

SEQ ID NO: 502 - protein, 20 aa, synthetic construct
RGKKTKKEQKTAYFLPMAIT

SEQ ID NO: 503 - protein, 25 aa, synthetic construct
SYARLDKQGNFNDWVAGSYGNDQWL

SEQ ID NO: 504 - protein, 25 aa, synthetic construct
SMVHPLTQTQDKVLPLEAEPPPGPP

SEQ ID NO: 505 - protein, 25 aa, synthetic construct
RRENEELSARYLLQCQEAEQARAEL

SEQ ID NO: 506 - protein, 25 aa, synthetic construct
GTFCVKPLKQKQIVDGVSYLLQEIY

SEQ ID NO: 507 - protein, 25 aa, synthetic construct
APHKKVGFVGISDWALDPAKMNRGI

SEQ ID NO: 508 - protein, 25 aa, synthetic construct
PSEVQHECIPQAFLGMDVLCQAKSG

SEQ ID NO: 509 - protein, 25 aa, synthetic construct
TRQMGLLFTWYDCLTGVPVSQQNLL

SEQ ID NO: 510 - protein, 25 aa, synthetic construct
LQRRGDCFAFMHQCIQEFCAAMFYL

SEQ ID NO: 511 - protein, 25 aa, synthetic construct
LLANDPTSLRKNFSIQRYAVIPLST

SEQ ID NO: 512 - protein, 25 aa, synthetic construct
HATEYAVGIYDNSMVPQMCGNLTPS

SEQ ID NO: 513 - protein, 25 aa, synthetic construct
PARASQDSADPQTPAQGNFRGSWDC

SEQ ID NO: 514 - protein, 25 aa, synthetic construct
LFGQSCLAALSTGCQNMIASLGAPN

SEQ ID NO: 515 - protein, 25 aa, synthetic construct
GLELLAASEAPRYPPGQAKPRGAPV

SEQ ID NO: 516 - protein, 25 aa, synthetic construct
NTFQGVIFQGSICYEALKKVYDNRV

SEQ ID NO: 517 - protein, 20 aa, synthetic construct
DKPYIDEARRNLTEWLNKYM

SEQ ID NO: 518 - protein, 25 aa, synthetic construct
TKYVHQELLNHKKSYVIKTTKLKTP

SEQ ID NO: 519 - protein, 25 aa, synthetic construct
KDPGNEVKLKLYGLYKQATEGPCNM

SEQ ID NO: 520 - protein, 25 aa, synthetic construct
CFSYAALLTKTNHIHRIFEQGKKSV

SEQ ID NO: 521 - protein, 25 aa, synthetic construct
AGAGLEPSSKKKLPSPRPGSPRVPP

SEQ ID NO: 522 - protein, 25 aa, synthetic construct
RFKKRTELFIAAKGIHTGQFVYCGK

SEQ ID NO: 523 - protein, 25 aa, synthetic construct
LFCSCQDQACAEHRRQTILPSCSYE

SEQ ID NO: 524 - protein, 25 aa, synthetic construct
RPHRPEWVHDKAYYMPETRLRIPAA

SEQ ID NO: 525 - protein, 25 aa, synthetic construct
LRTLNLGGNALDHVARAWFADLAEL

SEQ ID NO: 526 - protein, 25 aa, synthetic construct
VKIVQKNDPFVVEISLGVCRSLTVA

SEQ ID NO: 527 - protein, 25 aa, synthetic construct
KNDPFVVEISLGVCRSLTVACCTGG

SEQ ID NO: 528 - protein, 25 aa, synthetic construct
VVEISLGVCRSLTVACCTGGLVGGT

SEQ ID NO: 529 - protein, 25 aa, synthetic construct
LGVCRSLTVACCTGGLVGGTPLSIS

SEQ ID NO: 530 - protein, 25 aa, synthetic construct
SLTVACCTGGLVGGTPLSISRPTSR

SEQ ID NO: 531 - protein, 25 aa, synthetic construct
CCTGGLVGGTPLSISRPTSRARQSC

SEQ ID NO: 532 - protein, 25 aa, synthetic construct
LVGGTPLSISRPTSRARQSCCLPGL

SEQ ID NO: 533 - protein, 25 aa, synthetic construct
PLSISRPTSRARQSCCLPGLTHPAH

SEQ ID NO: 534 - protein, 25 aa, synthetic construct
ISRPTSRARQSCCLPGLTHPAHQPL

SEQ ID NO: 535 - protein, 25 aa, synthetic construct
PTSRARQSCCLPGLTHPAHQPLGSM

SEQ ID NO: 536 - protein, 6 aa, Homo sapiens
VSGNPY

SEQ ID NO: 537 - protein, 8 aa, Homo sapiens
YITGDNLV

SEQ ID NO: 538 - protein, 11 aa, Homo sapiens
AVSLFLDDKII

SEQ ID NO: 539 - protein, 121 aa, Homo sapiens
QSVAQPEDQVNVAEGNPLTVKCTYSVSGNPYLFWYVQYPNRGLQFLLKYITGDNLVKGSYGFEAEFNKSQTSFHLKKPSALVSDSALYFCAVSLFLDDKIIFGKGTRLHILPNIQNPEPAV

SEQ ID NO: 540 - protein, 141 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVSLFLDDKIIFGKGTRLHILPNIQNPEPAV

SEQ ID NO: 541 - protein, 278 aa, synthetic construct
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVSLFLDDKIIFGKGTRLHILPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 542 - protein, 5 aa, Homo sapiens
SGHRS

SEQ ID NO: 543 - protein, 6 aa, synthetic construct
YFSETQ

SEQ ID NO: 544 - protein, 12 aa, Homo sapiens
ASSLARVEDEAF

SEQ ID NO: 545 - protein, 116 aa, Homo sapiens
GVTQTPRYLIKTRGQQVTLSCSPISGHRSVSWYQQTPGQGLQFLFEYFSETQRNKGNFPGRFSGRQFSNSRSEMNVSTLELGDSALYLCASSLARVEDEAFFGQGTRLTVVEDLRN

SEQ ID NO: 546 - protein, 137 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLARVEDEAFFGQGTRLTVVEDLRN

SEQ ID NO: 547 - protein, 310 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLARVEDEAFFGQGTRLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 548 - protein, 6 aa, Homo sapiens
VSGNPY

SEQ ID NO: 549 - protein, 8 aa, Homo sapiens
YITGDNLV

SEQ ID NO: 550 - protein, 11 aa, Homo sapiens
AVRGFLINDMR

SEQ ID NO: 551 - protein, 121 aa, Homo sapiens
QSVAQPEDQVNVAEGNPLTVKCTYSVSGNPYLFWYVQYPNRGLQFLLKYITGDNLVKGSYGFEAEFNKSQTSFHLKKPSALVSDSALYFCAVRGFLINDMRFGAGTRLTVKPNIQNPEPAV

SEQ ID NO: 552 - protein, 141 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRGFLINDMRFGAGTRLTVKPNIQNPEPAV

SEQ ID NO: 553 - protein, 278 aa, synthetic construct
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRGFLINDMRFGAGTRLTVKPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 554 - protein, 5 aa, Homo sapiens
SGHRS

SEQ ID NO: 555 - protein, 6 aa, Homo sapiens
YFSETQ

SEQ ID NO: 556 - protein, 12 aa, Homo sapiens
ASSLGRVENEQY

SEQ ID NO: 557 - protein, 116 aa, Homo sapiens
GVTQTPRYLIKTRGQQVTLSCSPISGHRSVSWYQQTPGQGLQFLFEYFSETQRNKGNFPGRFSGRQFSNSRSEMNVSTLELGDSALYLCASSLGRVENEQYFGPGTRLTVTEDLRN

SEQ ID NO: 558 - protein, 137 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGRVENEQYFGPGTRLTVTEDLRN

SEQ ID NO: 559 - protein, 310 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGRVENEQYFGPGTRLTVTEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 560 - protein, 6 aa, Homo sapiens
SSVSVY

SEQ ID NO: 561 - protein, 8 aa, Homo sapiens
YLSGSTLV

SEQ ID NO: 562 - protein, 13 aa, Homo sapiens
AVMERGGSNYKLT

SEQ ID NO: 563 - protein, 123 aa, Homo sapiens
QSVTQLDSQVPVFEEAPVELRCNYSSSVSVYLFWYVQYPNQGLQLLLKYLSGSTLVKGINGFEAEFNKSQTSFHLRKPSVHISDTAEYFCAVMERGGSNYKLTFGKGTLLTVNPNIQNPEPAV

SEQ ID NO: 564 - protein, 143 aa, Homo sapiens
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVMERGGSNYKLTFGKGTLLTVNPNIQNPEPAV

SEQ ID NO: 565 - protein, 280 aa, synthetic construct
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVMERGGSNYKLTFGKGTLLTVNPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 566 - protein, 5 aa, Homo sapiens
LNHDA

SEQ ID NO: 567 - protein, 6 aa, Homo sapiens
SQIVND

SEQ ID NO: 568 - protein, 13 aa, Homo sapiens
ASSRDGYPGNTIY

SEQ ID NO: 569 - protein, 117 aa, Homo sapiens
GITQSPKYLFRKEGQNVTLSCEQNLNHDAMYWYRQDPGQGLRLIYYSQIVNDFQKGDIAEGYSVSREKKESFPLTVTSAQKNPTAFYLCASSRDGYPGNTIYFGEGSWLTVVEDLRN

SEQ ID NO: 570 - protein, 138 aa, Homo sapiens
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSRDGYPGNTIYFGEGSWLTVVEDLRN

SEQ ID NO: 571 - protein, 311 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSRDGYPGNTIYFGEGSWLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 572 - protein, 7 aa, Homo sapiens
TISGNEY

SEQ ID NO: 573 - protein, 5 aa, Homo sapiens
GLKNN

SEQ ID NO: 574 - protein, 11 aa, Homo sapiens
IVRPHNTGKLI

SEQ ID NO: 575 - protein, 117 aa, Homo sapiens
KTTQPPSMDCAEGRAANLPCNHSTISGNEYVYWYRQIHSQGPQYIIHGLKNNETNEMASLIITEDRKSSTLILPHATLRDTAVYYCIVRPHNTGKLIFGQGTTLQVKPNIQNPEPAV

SEQ ID NO: 576 - protein, 136 aa, Homo sapiens
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRPHNTGKLIFGQGTTLQVKPNIQNPEPAV

SEQ ID NO: 577 - protein, 273 aa, synthetic construct
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRPHNTGKLIFGQGTTLQVKPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 578 - protein, 5 aa, Homo sapiens
MNHNS

SEQ ID NO: 579 - protein, 6 aa, Homo sapiens
SASEGT

SEQ ID NO: 580 - protein, 13 aa, Homo sapiens
ASSEMDSGTDTQY

SEQ ID NO: 581 - protein, 117 aa, Homo sapiens
GVTQTPKFQVLKTGQSMTLQCAQDMNHNSMYWYRQDPGMGLRLIYYSASEGTTDKGEVPNGYNVSRLNKREFSLRLESAAPSQTSVYFCASSEMDSGTDTQYFGPGTRLTVLEDLRN

SEQ ID NO: 582 - protein, 138 aa, Homo sapiens
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEMDSGTDTQYFGPGTRLTVLEDLRN

SEQ ID NO: 583 - protein, 311 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEMDSGTDTQYFGPGTRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 584 - protein, 7 aa, Homo sapiens
TSESNYY

SEQ ID NO: 585 - protein, 8 aa, Homo sapiens
QEAYKQQN

SEQ ID NO: 586 - protein, 11 aa, Homo sapiens
ALKETSGSRLT

SEQ ID NO: 587 - protein, 122 aa, Homo sapiens
QTVTQSQPEMSVQEAETVTLSCTYDTSESNYYLFWYKQPPSRQMILVIRQEAYKQQNATENRFSVNFQKAAKSFSLKISDSQLGDTAMYFCALKETSGSRLTFGEGTQLTVNPNIQNPEPAV

SEQ ID NO: 588 - protein, 143 aa, Homo sapiens
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCALKETSGSRLTFGEGTQLTVNPNIQNPEPAV

SEQ ID NO: 589 - protein, 280 aa, synthetic construct
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCALKETSGSRLTFGEGTQLTVNPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 590 - protein, 5 aa, Homo sapiens
MGHRA

SEQ ID NO: 591 - protein, 6 aa, Homo sapiens
YSYEKL

SEQ ID NO: 592 - protein, 12 aa, Homo sapiens
ASSQDNTYNEQF

SEQ ID NO: 593 - protein, 116 aa, Homo sapiens
EVTQTPKHLVMGMTNKKSLKCEQHMGHRAMYWYKQKAKKPPELMFVYSYEKLSINESVPSRFSPECPNSSLLNLHLHALQPEDSALYLCASSQDNTYNEQFFGPGTRLTVLEDLRN

SEQ ID NO: 594 - protein, 137 aa, Homo sapiens
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQDNTYNEQFFGPGTRLTVLEDLRN

SEQ ID NO: 595 - protein, 310 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQDNTYNEQFFGPGTRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 596 - protein, 7 aa, Homo sapiens
TISGNEY

SEQ ID NO: 597 - protein, 5 aa, Homo sapiens
GLKNN

SEQ ID NO: 598 - protein, 11 aa, Homo sapiens
IVRAHNDYKLS

SEQ ID NO: 599 - protein, 117 aa, Homo sapiens
KTTQPPSMDCAEGRAANLPCNHSTISGNEYVYWYRQIHSQGPQYIIHGLKNNETNEMASLIITEDRKSSTLILPHATLRDTAVYYCIVRAHNDYKLSFGAGTTVTVRANIQNPEPAV

SEQ ID NO: 600 - protein, 136 aa, Homo sapiens
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRAHNDYKLSFGAGTTVTVRANIQNPEPAV

SEQ ID NO: 601 - protein, 273 aa, synthetic construct
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRAHNDYKLSFGAGTTVTVRANIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 602 - protein, 5 aa, Homo sapiens
MNHNS

SEQ ID NO: 603 - protein, 6 aa, Homo sapiens
SASEGT

SEQ ID NO: 604 - protein, 12 aa, Homo sapiens
ASSPDVGDYGYT

SEQ ID NO: 605 - protein, 116 aa, Homo sapiens
GVTQTPKFQVLKTGQSMTLQCAQDMNHNSMYWYRQDPGMGLRLIYYSASEGTTDKGEVPNGYNVSRLNKREFSLRLESAAPSQTSVYFCASSPDVGDYGYTFGSGTRLTVVEDLRN

SEQ ID NO: 606 - protein, 137 aa, Homo sapiens
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSPDVGDYGYTFGSGTRLTVVEDLRN

SEQ ID NO: 607 - protein, 310 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSPDVGDYGYTFGSGTRLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 608 - protein, 6 aa, Homo sapiens
SSVSVY

SEQ ID NO: 609 - protein, 8 aa, Homo sapiens
YLSGSTLV

SEQ ID NO: 610 - protein, 13 aa, Homo sapiens
AVSDHGFGNEKLT

SEQ ID NO: 611 - protein, 123 aa, Homo sapiens
QSVTQLDSQVPVFEEAPVELRCNYSSSVSVYLFWYVQYPNQGLQLLLKYLSGSTLVKGINGFEAEFNKSQTSFHLRKPSVHISDTAEYFCAVSDHGFGNEKLTFGTGTRLTIIPNIQNPEPAV

SEQ ID NO: 612 - protein, 143 aa, Homo sapiens
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVSDHGFGNEKLTFGTGTRLTIIPNIQNPEPAV

SEQ ID NO: 613 - protein, 280 aa, synthetic construct
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVSDHGFGNEKLTFGTGTRLTIIPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 614 - protein, 6 aa, Homo sapiens
DFQATT

SEQ ID NO: 615 - protein, 7 aa, Homo sapiens
SNEGSKA

SEQ ID NO: 616 - protein, 11 aa, Homo sapiens
SARRDRNQPQH

SEQ ID NO: 617 - protein, 119 aa, Homo sapiens
AVVSQHPSRVICKSGTSVKIECRSLDFQATTMFWYRQFPKQSLMLMATSNEGSKATYEQGVEKDKFLINHASLTLSTLTVTSAHPEDSSFYICSARRDRNQPQHFGDGTRLSILEDLRN

SEQ ID NO: 618 - protein, 134 aa, Homo sapiens
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSARRDRNQPQHFGDGTRLSILEDLRN

SEQ ID NO: 619 - protein, 307 aa, synthetic construct
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSARRDRNQPQHFGDGTRLSILEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 620 - protein, 6 aa, Homo sapiens
NSMFDY

SEQ ID NO: 621 - protein, 7 aa, Homo sapiens
ISSIKDK

SEQ ID NO: 622 - protein, 10 aa, Homo sapiens
AARSGTYKYI

SEQ ID NO: 623 - protein, 115 aa, Homo sapiens
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAARSGTYKYIFGTGTRLKVL

SEQ ID NO: 624 - protein, 136 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAARSGTYKYIFGTGTRLKVL

SEQ ID NO: 625 - protein, 273 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAARSGTYKYIFGTGTRLKVLNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 626 - protein, 5 aa, Homo sapiens
SGDLS

SEQ ID NO: 627 - protein, 6 aa, Homo sapiens
YYNGEE

SEQ ID NO: 628 - protein, 13 aa, Homo sapiens
ASSEGVGQIYGYT

SEQ ID NO: 629 - protein, 112 aa, Homo sapiens
GVTQTPKHLITATGQRVTLRCSPRSGDLSVYWYQQSLDQGLQFLIQYYNGEERAKGNILERFSAQQFPDLHSELNLSSLELGDSALYFCASSEGVGQIYGYTFGSGTRLTVV

SEQ ID NO: 630 - protein, 133 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSEGVGQIYGYTFGSGTRLTVV

SEQ ID NO: 631 - protein, 306 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSEGVGQIYGYTFGSGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 632 - protein, 7 aa, Homo sapiens
TISGTDY

SEQ ID NO: 633 - protein, 5 aa, Homo sapiens
GLTSN

SEQ ID NO: 634 - protein, 11 aa, Homo sapiens
ILFSGNTGKLI

SEQ ID NO: 635 - protein, 108 aa, Homo sapiens
KTTQPNSMESNEEEPVHLPCNHSTISGTDYIHWYRQLPSQGPEYVIHGLTSNVNNRMASLAIAEDRKSSTLILHRATLRDAAVYYCILFSGNTGKLIFGQGTTLQVKP

SEQ ID NO: 636 - protein, 127 aa, Homo sapiens
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILFSGNTGKLIFGQGTTLQVKP

SEQ ID NO: 637 - protein, 264 aa, synthetic construct
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILFSGNTGKLIFGQGTTLQVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 638 - protein, 5 aa, Homo sapiens
LGHNA

SEQ ID NO: 639 - protein, 6 aa, Homo sapiens
YSLEER

SEQ ID NO: 640 - protein, 13 aa, Homo sapiens
ASSQDRGDLYGYT

SEQ ID NO: 641 - protein, 112 aa, Homo sapiens
GVTQTPRHLVMGMTNKKSLKCEQHLGHNAMYWYKQSAKKPLELMFVYSLEERVENNSVPSRFSPECPNSSHLFLHLHTLQPEDSALYLCASSQDRGDLYGYTFGSGTRLTVV

SEQ ID NO: 642 - protein, 133 aa, Homo sapiens
MGCRLLCCAVLCLLGAVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDRGDLYGYTFGSGTRLTVV

SEQ ID NO: 643 - protein, 306 aa, synthetic construct
MGCRLLCCAVLCLLGAVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDRGDLYGYTFGSGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 644 - protein, 6 aa, Homo sapiens
DSAIYN

SEQ ID NO: 645 - protein, 7 aa, Homo sapiens
IQSSQRE

SEQ ID NO: 646 - protein, 10 aa, Homo sapiens
APRGFGNVLH

SEQ ID NO: 647 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAPRGFGNVLHCGSGTQVIVLP

SEQ ID NO: 648 - protein, 130 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAPRGFGNVLHCGSGTQVIVLP

SEQ ID NO: 649 - protein, 267 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAPRGFGNVLHCGSGTQVIVLPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 650 - protein, 5 aa, Homo sapiens
MNHEY

SEQ ID NO: 651 - protein, 6 aa, Homo sapiens
SMNVEV

SEQ ID NO: 652 - protein, 11 aa, Homo sapiens
ASSLLGGETQY

SEQ ID NO: 653 - protein, 110 aa, Homo sapiens
QVTQNPRYLITVTGKKLTVTCSQNMNHEYMSWYRQDPGLGLRQIYYSMNVEVTDKGDVPEGYKVSRKEKRNFPLILESPSPNQTSLYFCASSLLGGETQYFGPGTRLLVL

SEQ ID NO: 654 - protein, 131 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLLGGETQYFGPGTRLLVL

SEQ ID NO: 655 - protein, 304 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLLGGETQYFGPGTRLLVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 656 - protein, 6 aa, Homo sapiens
YGATPY

SEQ ID NO: 657 - protein, 8 aa, Homo sapiens
YFSGDTLV

SEQ ID NO: 658 - protein, 11 aa, Homo sapiens
AADQTGANNLF

SEQ ID NO: 659 - protein, 112 aa, synthetic construct
QSVTQPDIHITVSEGASLELRCNYSYGATPYLFWYVQSPGQGLQLLLKYFSGDTLVQGIKGFEAEFKRSQSSFNLRKPSVHWSDAAEYFCAADQTGANNLFFGTGTRLTVIP

SEQ ID NO: 660 - protein, 132 aa, Homo sapiens
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAADQTGANNLFFGTGTRLTVIP

SEQ ID NO: 661 - protein, 269 aa, synthetic construct
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAADQTGANNLFFGTGTRLTVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 662 - protein, 5 aa, Homo sapiens
MNHEY

SEQ ID NO: 663 - protein, 6 aa, Homo sapiens
SVGEGT

SEQ ID NO: 664 - protein, 9 aa, Homo sapiens
ASSPRGGYT

SEQ ID NO: 665 - protein, 108 aa, Homo sapiens
GVTQTPKFRVLKTGQSMTLLCAQDMNHEYMYWYRQDPGMGLRLIHYSVGEGTTAKGEVPDGYNVSRLKKQNFLLGLESAAPSQTSVYFCASSPRGGYTFGSGTRLTVV

SEQ ID NO: 666 - protein, 129 aa, Homo sapiens
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSPRGGYTFGSGTRLTVV

SEQ ID NO: 667 - protein, 302 aa, synthetic construct
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSPRGGYTFGSGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 668 - protein, 6 aa, Homo sapiens
VSNAYN

SEQ ID NO: 669 - protein, 4 aa, Homo sapiens
GSKP

SEQ ID NO: 670 - protein, 9 aa, Homo sapiens
AREAGTALI

SEQ ID NO: 671 - protein, 108 aa, Homo sapiens
VAESKDQVFQPSTVASSEGAVVEIFCNHSVSNAYNFFWYLHFPGCAPRLLVKGSKPSQQGRYNMTYERFSSSLLILQVREADAAVYYCAREAGTALIFGKGTTLSVSS

SEQ ID NO: 672 - protein, 129 aa, Homo sapiens
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAREAGTALIFGKGTTLSVSS

SEQ ID NO: 673 - protein, 266 aa, synthetic construct
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAREAGTALIFGKGTTLSVSSNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 674 - protein, 5 aa, Homo sapiens
MNHNS

SEQ ID NO: 675 - protein, 6 aa, Homo sapiens
SASEGT

SEQ ID NO: 676 - protein, 12 aa, Homo sapiens
ASRQDGSNQPQH

SEQ ID NO: 677 - protein, 111 aa, Homo sapiens
GVTQTPKFQVLKTGQSMTLQCAQDMNHNSMYWYRQDPGMGLRLIYYSASEGTTDKGEVPNGYNVSRLNKREFSLRLESAAPSQTSVYFCASRQDGSNQPQHFGDGTRLSIL

SEQ ID NO: 678 - protein, 132 aa, Homo sapiens
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRQDGSNQPQHFGDGTRLSIL

SEQ ID NO: 679 - protein, 305 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRQDGSNQPQHFGDGTRLSILEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 680 - protein, 6 aa, Homo sapiens
YGATPY

SEQ ID NO: 681 - protein, 8 aa, Homo sapiens
YFSGDTLV

SEQ ID NO: 682 - protein, 10 aa, Homo sapiens
AVHSNDYKLS

SEQ ID NO: 683 - protein, 111 aa, Homo sapiens
QSVTQPDIHITVSEGASLELRCNYSYGATPYLFWYVQSPGQGLQLLLKYFSGDTLVQGIKGFEAEFKRSQSSFNLRKPSVHWSDAAEYFCAVHSNDYKLSFGAGTTVTVRA

SEQ ID NO: 684 - protein, 131 aa, Homo sapiens
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVHSNDYKLSFGAGTTVTVRA

SEQ ID NO: 685 - protein, 268 aa, synthetic construct
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVHSNDYKLSFGAGTTVTVRANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 686 - protein, 5 aa, Homo sapiens
SGHTA

SEQ ID NO: 687 - protein, 6 aa, Homo sapiens
FQGNSA

SEQ ID NO: 688 - protein, 10 aa, Homo sapiens
ASSRRGTEAF

SEQ ID NO: 689 - protein, 110 aa, Homo sapiens
GVSQSPSNKVTEKGKDVELRCDPISGHTALYWYRQSLGQGLEFLIYFQGNSAPDKSGLPSDRFSAERTGGSVSTLTIQRTQQEDSAVYLCASSRRGTEAFFGQGTRLTVV

SEQ ID NO: 690 - protein, 131 aa, Homo sapiens
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSRRGTEAFFGQGTRLTVV

SEQ ID NO: 691 - protein, 304 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSRRGTEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 692 - protein, 7 aa, Homo sapiens
NIATNDY

SEQ ID NO: 693 - protein, 5 aa, Homo sapiens
GYKTK

SEQ ID NO: 694 - protein, 16 aa, Homo sapiens
LVGDIGYSGGGADGLT

SEQ ID NO: 695 - protein, 113 aa, Homo sapiens
KTTQPISMDSYEGQEVNITCSHNNIATNDYITWYQQFPSQGPRFIIQGYKTKVTNEVASLFIPADRKSSTLSLPRVSLSDTAVYYCLVGDIGYSGGGADGLTFGKGTHLIIQP

SEQ ID NO: 696 - protein, 132 aa, Homo sapiens
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDIGYSGGGADGLTFGKGTHLIIQP

SEQ ID NO: 697 - protein, 269 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDIGYSGGGADGLTFGKGTHLIIQPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 698 - protein, 5 aa, Homo sapiens
SNHLY

SEQ ID NO: 699 - protein, 6 aa, Homo sapiens
FYNNEI

SEQ ID NO: 700 - protein, 12 aa, Homo sapiens
ASSEQGAGDTQY

SEQ ID NO: 701 - protein, 114 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSEQGAGDTQYFGPGTRLTVL

SEQ ID NO: 702 - protein, 133 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEQGAGDTQYFGPGTRLTVL

SEQ ID NO: 703 - protein, 306 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEQGAGDTQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 704 - protein, 6 aa, Homo sapiens
NSAFQY

SEQ ID NO: 705 - protein, 6 aa, Homo sapiens
TYSSGN

SEQ ID NO: 706 - protein, 14 aa, Homo sapiens
AMSEHYGGSQGNLI

SEQ ID NO: 707 - protein, 115 aa, Homo sapiens
QQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEHYGGSQGNLIFGKGTKLSVKP

SEQ ID NO: 708 - protein, 136 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEHYGGSQGNLIFGKGTKLSVKP

SEQ ID NO: 709 - protein, 273 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEHYGGSQGNLIFGKGTKLSVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 710 - protein, 5 aa, Homo sapiens
SGHDY

SEQ ID NO: 711 - protein, 6 aa, Homo sapiens
FNNNVP

SEQ ID NO: 712 - protein, 11 aa, Homo sapiens
ASSYGAGGPQH

SEQ ID NO: 713 - protein, 111 aa, Homo sapiens
GVIQSPRHEVTEMGQEVTLRCKPISGHDYLFWYRQTMMRGLELLIYFNNNVPIDDSGMPEDRFSAKMPNASFSTLKIQPSEPRDSAVYFCASSYGAGGPQHFGDGTRLSIL

SEQ ID NO: 714 - protein, 132 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSYGAGGPQHFGDGTRLSIL

SEQ ID NO: 715 - protein, 305 aa, synthetic construct
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSYGAGGPQHFGDGTRLSILEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 716 - protein, 5 aa, Homo sapiens
DSVNN

SEQ ID NO: 717 - protein, 5 aa, Homo sapiens
IPSGT

SEQ ID NO: 718 - protein, 14 aa, Homo sapiens
AVKSKSGGSNYKLT

SEQ ID NO: 719 - protein, 111 aa, Homo sapiens
IQVEQSPPDLILQEGANSTLRCNFSDSVNNLQWFHQNPWGQLINLFYIPSGTKQNGRLSATTVATERYSLLYISSSQTTDSGVYFCAVKSKSGGSNYKLTFGKGTLLTVNP

SEQ ID NO: 720 - protein, 132 aa, Homo sapiens
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVKSKSGGSNYKLTFGKGTLLTVNP

SEQ ID NO: 721 - protein, 269 aa, synthetic construct
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVKSKSGGSNYKLTFGKGTLLTVNPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 722 - protein, 5 aa, Homo sapiens
SNHLY

SEQ ID NO: 723 - protein, 6 aa, Homo sapiens
FYNNEI

SEQ ID NO: 724 - protein, 9 aa, Homo sapiens
ASSATGYAF

SEQ ID NO: 725 - protein, 111 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSATGYAFFGQGTRLTVV

SEQ ID NO: 726 - protein, 130 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSATGYAFFGQGTRLTVV

SEQ ID NO: 727 - protein, 303 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSATGYAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 728 - protein, 5 aa, Homo sapiens
DSVNN

SEQ ID NO: 729 - protein, 5 aa, Homo sapiens
IPSGT

SEQ ID NO: 730 - protein, 13 aa, Homo sapiens
AVDGYGGSQGNLI

SEQ ID NO: 731 - protein, 110 aa, Homo sapiens
IQVEQSPPDLILQEGANSTLRCNFSDSVNNLQWFHQNPWGQLINLFYIPSGTKQNGRLSATTVATERYSLLYISSSQTTDSGVYFCAVDGYGGSQGNLIFGKGTKLSVKP

SEQ ID NO: 732 - protein, 131 aa, Homo sapiens
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVDGYGGSQGNLIFGKGTKLSVKP

SEQ ID NO: 733 - protein, 268 aa, synthetic construct
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVDGYGGSQGNLIFGKGTKLSVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 734 - protein, 5 aa, Homo sapiens
SNHLY

SEQ ID NO: 735 - protein, 6 aa, Homo sapiens
FYNNEI

SEQ ID NO: 736 - protein, 9 aa, Homo sapiens
ASRGDTEAF

SEQ ID NO: 737 - protein, 111 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFCASRGDTEAFFGQGTRLTVV

SEQ ID NO: 738 - protein, 130 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASRGDTEAFFGQGTRLTVV

SEQ ID NO: 739 - protein, 303 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASRGDTEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 740 - protein, 5 aa, Homo sapiens
DSVNN

SEQ ID NO: 741 - protein, 5 aa, Homo sapiens
IPSGT

SEQ ID NO: 742 - protein, 14 aa, Homo sapiens
AVDRKSGGSNYKLT

SEQ ID NO: 743 - protein, 111 aa, Homo sapiens
IQVEQSPPDLILQEGANSTLRCNFSDSVNNLQWFHQNPWGQLINLFYIPSGTKQNGRLSATTVATERYSLLYISSSQTTDSGVYFCAVDRKSGGSNYKLTFGKGTLLTVNP

SEQ ID NO: 744 - protein, 132 aa, Homo sapiens
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVDRKSGGSNYKLTFGKGTLLTVNP

SEQ ID NO: 745 - protein, 269 aa, synthetic construct
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVDRKSGGSNYKLTFGKGTLLTVNPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 746 - protein, 5 aa, Homo sapiens
SGHNS

SEQ ID NO: 747 - protein, 6 aa, Homo sapiens
FNNNVP

SEQ ID NO: 748 - protein, 9 aa, Homo sapiens
ASSLSSEAF

SEQ ID NO: 749 - protein, 109 aa, Homo sapiens
GVIQSPRHEVTEMGQEVTLRCKPISGHNSLFWYRQTMMRGLELLIYFNNNVPIDDSGMPEDRFSAKMPNASFSTLKIQPSEPRDSAVYFCASSLSSEAFFGQGTRLTVV

SEQ ID NO: 750 - protein, 130 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLSSEAFFGQGTRLTVV

SEQ ID NO: 751 - protein, 303 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLSSEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 752 - protein, 7 aa, Homo sapiens
TSESNYY

SEQ ID NO: 753 - protein, 8 aa, Homo sapiens
QEAYKQQN

SEQ ID NO: 754 - protein, 17 aa, Homo sapiens
AFMKPHPAGGTSYGKLT

SEQ ID NO: 755 - protein, 119 aa, Homo sapiens
QTVTQSQPEMSVQEAETVTLSCTYDTSESNYYLFWYKQPPSRQMILVIRQEAYKQQNATENRFSVNFQKAAKSFSLKISDSQLGDTAMYFCAFMKPHPAGGTSYGKLTFGQGTILTVHP

SEQ ID NO: 756 - protein, 140 aa, Homo sapiens
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFMKPHPAGGTSYGKLTFGQGTILTVHP

SEQ ID NO: 757 - protein, 140 aa, synthetic construct; identical to SEQ ID NO: 756

SEQ ID NO: 758 - protein, 5 aa, Homo sapiens
MNHEY

SEQ ID NO: 759 - protein, 6 aa, Homo sapiens
SVGAGI

SEQ ID NO: 760 - protein, 14 aa, Homo sapiens
ASRVGRSVGTGELF

SEQ ID NO: 761 - protein, 113 aa, Homo sapiens
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASRVGRSVGTGELFFGEGSRLTVL

SEQ ID NO: 762 - protein, 134 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRVGRSVGTGELFFGEGSRLTVL

SEQ ID NO: 763 - protein, 307 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRVGRSVGTGELFFGEGSRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 764 - protein, 7 aa, Homo sapiens
TSESNYY

SEQ ID NO: 765 - protein, 8 aa, Homo sapiens
QEAYKQQN

SEQ ID NO: 766 - protein, 12 aa, Homo sapiens
AFMTPNNNNDMR

SEQ ID NO: 767 - protein, 114 aa, Homo sapiens
QTVTQSQPEMSVQEAETVTLSCTYDTSESNYYLFWYKQPPSRQMILVIRQEAYKQQNATENRFSVNFQKAAKSFSLKISDSQLGDTAMYFCAFMTPNNNNDMRFGAGTRLTVKP

SEQ ID NO: 768 - protein, 135 aa, Homo sapiens
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFMTPNNNNDMRFGAGTRLTVKP

SEQ ID NO: 769 - protein, 272 aa, synthetic construct
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFMTPNNNNDMRFGAGTRLTVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 770 - protein, 5 aa, Homo sapiens
KGHSH

SEQ ID NO: 771 - protein, 6 aa, Homo sapiens
LQKENI

SEQ ID NO: 772 - protein, 11 aa, Homo sapiens
ASSPGSYSPLH

SEQ ID NO: 773 - protein, 111 aa, Homo sapiens
GVMQNPRHLVRRRGQEARLRCSPMKGHSHVYWYRQLPEEGLKFMVYLQKENIIDESGMPKERFSAEFPKEGPSILRIQQVVRGDSAAYFCASSPGSYSPLHFGNGTRLTVT

SEQ ID NO: 774 - protein, 132 aa, Homo sapiens
MDTRLLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPGSYSPLHFGNGTRLTVT

SEQ ID NO: 775 - protein, 305 aa, synthetic construct
MDTRLLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPGSYSPLHFGNGTRLTVTEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 776 - protein, 6 aa, Homo sapiens
NSASQS

SEQ ID NO: 777 - protein, 6 aa, Homo sapiens
VYSSGN

SEQ ID NO: 778 - protein, 13 aa, Homo sapiens
VVNSGGGSQGNLI

SEQ ID NO: 779 - protein, 113 aa, Homo sapiens
QRKEVEQDPGPFNVPEGATVAFNCTYSNSASQSFFWYRQDCRKEPKLLMSVYSSGNEDGRFTAQLNRASQYISLLIRDSKLSDSATYLCVVNSGGGSQGNLIFGKGTKLSVKP

SEQ ID NO: 780 - protein, 133 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNSGGGSQGNLIFGKGTKLSVKP

SEQ ID NO: 781 - protein, 270 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNSGGGSQGNLIFGKGTKLSVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 782 - protein, 5 aa, Homo sapiens
LNHDA

SEQ ID NO: 783 - protein, 6 aa, Homo sapiens
SQIVND

SEQ ID NO: 784 - protein, 13 aa, Homo sapiens
ASSILTGNNSPLH

SEQ ID NO: 785 - protein, 112 aa, Homo sapiens
GITQSPKYLFRKEGQNVTLSCEQNLNHDAMYWYRQDPGQGLRLIYYSQIVNDFQKGDIAEGYSVSREKKESFPLTVTSAQKNPTAFYLCASSILTGNNSPLHFGNGTRLTVT

SEQ ID NO: 786 - protein, 133 aa, Homo sapiens
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSILTGNNSPLHFGNGTRLTVT

SEQ ID NO: 787 - protein, 306 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSILTGNNSPLHFGNGTRLTVTEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 788 - protein, 6 aa, Homo sapiens
NSAFQY

SEQ ID NO: 789 - protein, 6 aa, Homo sapiens
TYSSGN

SEQ ID NO: 790 - protein, 12 aa, Homo sapiens
AMSRSDTGNQFY

SEQ ID NO: 791 - protein, 113 aa, Homo sapiens
QQKEVEQDPGPLSVPEGAIVSLNCTYSNSAFQYFMWYRQYSRKGPELLMYTYSSGNKEDGRFTAQVDKSSKYISLFIRDSQPSDSATYLCAMSRSDTGNQFYFGTGTSLTVIP

SEQ ID NO: 792 - protein, 134 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSRSDTGNQFYFGTGTSLTVIP

SEQ ID NO: 793 - protein, 271 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSRSDTGNQFYFGTGTSLTVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 794 - protein, 5 aa, Homo sapiens
SGHAT

SEQ ID NO: 795 - protein, 6 aa, Homo sapiens
FQNNGV

SEQ ID NO: 796 - protein, 13 aa, Homo sapiens
ASSFGPGVTDTQY

SEQ ID NO: 797 - protein, 113 aa, Homo sapiens
GVAQSPRYKIIEKRQSVAFWCNPISGHATLYWYQQILGQGPKLLIQFQNNGVVDDSQLPKDRFSAERLKGVDSTLKIQPAKLEDSAVYLCASSFGPGVTDTQYFGPGTRLTVL

SEQ ID NO: 798 - protein, 134 aa, Homo sapiens
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSFGPGVTDTQYFGPGTRLTVL

SEQ ID NO: 799 - protein, 307 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSFGPGVTDTQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 800 - protein, 6 aa, Homo sapiens
VSGNPY

SEQ ID NO: 801 - protein, 8 aa, Homo sapiens
YITGDNLV

SEQ ID NO: 802 - protein, 13 aa, Homo sapiens
AVRDYFGNTGKLI

SEQ ID NO: 803 - protein, 123 aa, Homo sapiens
QSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDYFGNTGKLIFGQGTTLQVKPNIQNPEPAV

SEQ ID NO: 804 - protein, 143 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDYFGNTGKLIFGQGTTLQVKPNIQNPEPAV

SEQ ID NO: 805 - protein, 280 aa, synthetic construct
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDYFGNTGKLIFGQGTTLQVKPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 806 - protein, 5 aa, Homo sapiens
SQVTM

SEQ ID NO: 807 - protein, 7 aa, Homo sapiens
ANQGSEA

SEQ ID NO: 808 - protein, 11 aa, Homo sapiens
SVALTENTEAF

SEQ ID NO: 809 - protein, 118 aa, Homo sapiens
AVISQKPSRDICQRGTSLTIQCQVDSQVTMMFWYRQQPGQSLTLIATANQGSEATYESGFVIDKFPISRPNLTFSTLTVSNMSPEDSSIYLCSVALTENTEAFFGQGTRLTVVEDLRN

SEQ ID NO: 810 - protein, 134 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVALTENTEAFFGQGTRLTVVEDLRN

SEQ ID NO: 811 - protein, 307 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVALTENTEAFFGQGTRLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 812 - protein, 6 aa, Homo sapiens
TSGFNG

SEQ ID NO: 813 - protein, 6 aa, Homo sapiens
NVLDGL

SEQ ID NO: 814 - protein, 11 aa, Homo sapiens
AVRGDSWGKLQ

SEQ ID NO: 815 - protein, 118 aa, Homo sapiens
QNIDQPTEMTATEGAIVQINCTYQTSGFNGLFWYQQHAGEAPTFLSYNVLDGLEEKGRFSSFLSRSKGYSYLLLKELQMKDSASYLCAVRGDSWGKLQFGAGTQVVVTPNIQNPEPAV

SEQ ID NO: 816 - protein, 136 aa, Homo sapiens
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRGDSWGKLQFGAGTQVVVTPNIQNPEPAV

SEQ ID NO: 817 - protein, 273 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRGDSWGKLQFGAGTQVVVTPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 818 - protein, 5 aa, Homo sapiens
SGHTA

SEQ ID NO: 819 - protein, 6 aa, Homo sapiens
FQGNSA

SEQ ID NO: 820 - protein, 13 aa, Homo sapiens
ASSPAAGDEHEQY

SEQ ID NO: 821 - protein, 118 aa, Homo sapiens
GVSQSPSNKVTEKGKDVELRCDPISGHTALYWYRQSLGQGLEFLIYFQGNSAPDKSGLPSDRFSAERTGGSVSTLTIQRTQQEDSAVYLCASSPAAGDEHEQYFGPGTRLTVTEDLRN

SEQ ID NO: 822 - protein, 139 aa, Homo sapiens
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSPAAGDEHEQYFGPGTRLTVTEDLRN

SEQ ID NO: 823 - protein, 312 aa, synthetic construct
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSPAAGDEHEQYFGPGTRLTVTEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 824 - protein, 6 aa, Homo sapiens
NSASDY

SEQ ID NO: 825 - protein, 7 aa, Homo sapiens
IRSNMDK

SEQ ID NO: 826 - protein, 12 aa, Homo sapiens
AENPGGGADGLT

SEQ ID NO: 827 - protein, 119 aa, Homo sapiens
VGLHLPTLSVQEGDNSIINCAYSNSASDYFIWYKQESGKGPQFIIDIRSNMDKRQGQRVTVLLNKTVKHLSLQIAATQPGDSAVYFCAENPGGGADGLTFGKGTHLIIQPNIQNPEPAV

SEQ ID NO: 828 - protein, 142 aa, Homo sapiens
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENPGGGADGLTFGKGTHLIIQPNIQNPEPAV

SEQ ID NO: 829 - protein, 279 aa, synthetic construct
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENPGGGADGLTFGKGTHLIIQPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 830 - protein, 5 aa, Homo sapiens
LGHDT

SEQ ID NO: 831 - protein, 6 aa, Homo sapiens
YNNKEL

SEQ ID NO: 832 - protein, 12 aa, Homo sapiens
ASSPGTGGYGYT

SEQ ID NO: 833 - protein, 113 aa, Homo sapiens
QTPKYLVTQMGNDKSIKCEQNLGHDTMYWYKQDSKKFLKIMFSYNNKELIINETVPNRFSPKSPDKAHLNLHINSLELGDSAVYFCASSPGTGGYGYTFGSGTRLTVVEDLRN

SEQ ID NO: 834 - protein, 137 aa, Homo sapiens
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSPGTGGYGYTFGSGTRLTVVEDLRN

SEQ ID NO: 835 - protein, 310 aa, synthetic construct
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSPGTGGYGYTFGSGTRLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 836 - protein, 6 aa, Homo sapiens
DSASNY

SEQ ID NO: 837 - protein, 7 aa, Homo sapiens
IRSNVGE

SEQ ID NO: 838 - protein, 10 aa, Homo sapiens
GGGGATNKLI

SEQ ID NO: 839 - protein, 119 aa, Homo sapiens
ENVEQHPSTLSVQEGDSAVIKCTYSDSASNYFPWYKQELGKRPQLIIDIRSNVGEKKDQRIAVTLNKTAKHFSLHITETQPEDSAVYFCGGGGATNKLIFGTGTLLAVQPNIQNPEPAV

SEQ ID NO: 840 - protein, 139 aa, Homo sapiens
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCGGGGATNKLIFGTGTLLAVQPNIQNPEPAV

SEQ ID NO: 841 - protein, 276 aa, synthetic construct
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCGGGGATNKLIFGTGTLLAVQPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 842 - protein, 5 aa, Homo sapiens
SGHVS

SEQ ID NO: 843 - protein, 6 aa, Homo sapiens
FQNEAQ

SEQ ID NO: 844 - protein, 12 aa, Homo sapiens
ASNNEDGSYEQY

SEQ ID NO: 845 - protein, 117 aa, Homo sapiens
GVSQSPRYKVAKRGQDVALRCDPISGHVSLFWYQQALGQGPEFLTYFQNEAQLDKSGLPSDRFFAERPEGSVSTLKIQRTQQEDSAVYLCASNNEDGSYEQYFGPGTRLTVTEDLRN

SEQ ID NO: 846 - protein, 138 aa, Homo sapiens
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASNNEDGSYEQYFGPGTRLTVTEDLRN

SEQ ID NO: 847 - protein, 311 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASNNEDGSYEQYFGPGTRLTVTEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 848 - protein, 5 aa, Homo sapiens
TSINN

SEQ ID NO: 849 - protein, 7 aa, Homo sapiens
IRSNERE

SEQ ID NO: 850 - protein, 8 aa, Homo sapiens
ATGDDKII

SEQ ID NO: 851 - protein, 116 aa, Homo sapiens
QQGEEDPQALSIQEGENATMNCSYKTSINNLQWYRQNSGRGLVHLILIRSNEREKHSGRLRVTLDTSKKSSSLLITASRAADTASYFCATGDDKIIFGKGTRLHILPNIQNPEPAV

SEQ ID NO: 852 - protein, 137 aa, Homo sapiens
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATGDDKIIFGKGTRLHILPNIQNPEPAV

SEQ ID NO: 853 - protein, 274 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATGDDKIIFGKGTRLHILPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 854 - protein, 5 aa, Homo sapiens
LGHDT

SEQ ID NO: 855 - protein, 6 aa, Homo sapiens
YNNKEL

SEQ ID NO: 856 - protein, 12 aa, Homo sapiens
ASSQATGGEEAF

SEQ ID NO: 857 - protein, 113 aa, Homo sapiens
QTPKYLVTQMGNDKSIKCEQNLGHDTMYWYKQDSKKFLKIMFSYNNKELIINETVPNRFSPKSPDKAHLNLHINSLELGDSAVYFCASSQATGGEEAFFGQGTRLTVVEDLRN

SEQ ID NO: 858 - protein, 137 aa, Homo sapiens
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQATGGEEAFFGQGTRLTVVEDLRN

SEQ ID NO: 859 - protein, 310 aa, synthetic construct
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQATGGEEAFFGQGTRLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 860 - protein, 6 aa, Homo sapiens
ATGYPS

SEQ ID NO: 861 - protein, 7 aa, Homo sapiens
ATKADDK

SEQ ID NO: 862 - protein, 15 aa, Homo sapiens
ALSDPFAQGGSEKLV

SEQ ID NO: 863 - protein, 124 aa, Homo sapiens
DSVTQMEGPVTLSEEAFLTINCTYTATGYPSLFWYVQYPGEGLQLLLKATKADDKGSNKGFEATYRKETTSFHLEKGSVQVSDSAVYFCALSDPFAQGGSEKLVFGKGTKLTVNPNIQNPEPAV

SEQ ID NO: 864 - protein, 144 aa, Homo sapiens
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSDPFAQGGSEKLVFGKGTKLTVNPNIQNPEPAV

SEQ ID NO: 865 - protein, 281 aa, synthetic construct
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSDPFAQGGSEKLVFGKGTKLTVNPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 866 - protein, 5 aa, Homo sapiens
SGHAT

SEQ ID NO: 867 - protein, 6 aa, Homo sapiens
FQNNGV

SEQ ID NO: 868 - protein, 13 aa, Homo sapiens
ASSPRGGGNSPLH

SEQ ID NO: 869 - protein, 118 aa, Homo sapiens
GVAQSPRYKIIEKRQSVAFWCNPISGHATLYWYQQILGQGPKLLIQFQNNGVVDDSQLPKDRFSAERLKGVDSTLKIQPAKLEDSAVYLCASSPRGGGNSPLHFGNGTRLTVTEDLRN

SEQ ID NO: 870 - protein, 139 aa, Homo sapiens
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSPRGGGNSPLHFGNGTRLTVTEDLRN

SEQ ID NO: 871 - protein, 312 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSPRGGGNSPLHFGNGTRLTVTEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 872 - protein, 6 aa, Homo sapiens
DRGSQS

SEQ ID NO: 873 - protein, 6 aa, Homo sapiens
IYSNGD

SEQ ID NO: 874 - protein, 11 aa, Homo sapiens
AVHMDSNYQLI

SEQ ID NO: 875 - protein, 121 aa, Homo sapiens
QQKEVEQNSGPLSVPEGAIASLNCTYSDRGSQSFFWYRQYSGKSPELIMFIYSNGDKEDGRFTAQLNKASQYVSLLIRDSQPSDSATYLCAVHMDSNYQLIWGAGTKLIIKPNIQNPEPAV

SEQ ID NO: 876 - protein, 141 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVHMDSNYQLIWGAGTKLIIKPNIQNPEPAV

SEQ ID NO: 877 - protein, 278 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVHMDSNYQLIWGAGTKLIIKPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 878 - protein, 5 aa, Homo sapiens
LNHDA

SEQ ID NO: 879 - protein, 6 aa, Homo sapiens
SQIVND

SEQ ID NO: 880 - protein, 12 aa, Homo sapiens
ASSIQGSNTEAF

SEQ ID NO: 881 - protein, 117 aa, Homo sapiens
GGITQSPKYLFRKEGQNVTLSCEQNLNHDAMYWYRQDPGQGLRLIYYSQIVNDFQKGDIAEGYSVSREKKESFPLTVTSAQKNPTAFYLCASSIQGSNTEAFFGQGTRLTVVEDLRN

SEQ ID NO: 882 - protein, 137 aa, Homo sapiens
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIQGSNTEAFFGQGTRLTVVEDLRN

SEQ ID NO: 883 - protein, 310 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIQGSNTEAFFGQGTRLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 884 - protein, 6 aa, Homo sapiens
TSGFYG

SEQ ID NO: 885 - protein, 6 aa, Homo sapiens
NALDGL

SEQ ID NO: 886 - protein, 10 aa, Homo sapiens
AVGDSSYKLI

SEQ ID NO: 887 - protein, 117 aa, Homo sapiens
QSLEQPSEVTAVEGAIVQINCTYQTSGFYGLSWYQQHDGGAPTFLSYNALDGLEETGRFSSFLSRSDSYGYLLLQELQMKDSASYFCAVGDSSYKLIFGSGTRLLVRPNIQNPEPAV

SEQ ID NO: 888 - protein, 135 aa, Homo sapiens
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVGDSSYKLIFGSGTRLLVRPNIQNPEPAV

SEQ ID NO: 889 - protein, 272 aa, synthetic construct
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVGDSSYKLIFGSGTRLLVRPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 890 - protein, 5 aa, Homo sapiens
MDHEN

SEQ ID NO: 891 - protein, 6 aa, Homo sapiens
SYDVKM

SEQ ID NO: 892 - protein, 14 aa, Homo sapiens
ASSFKGLEATDTQY

SEQ ID NO: 893 - protein, 113 aa, Homo sapiens
SRYLVKRTGEKVFLECVQDMDHENMFWYRQDPGLGLRLIYFSYDVKMKEKGDIPEGYSVSREKKERFSLILESASTNQTSMYLCASSFKGLEATDTQYFGPGTRLTVLEDLRN

SEQ ID NO: 894 - protein, 139 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFKGLEATDTQYFGPGTRLTVLEDLRN

SEQ ID NO: 895 - protein, 312 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFKGLEATDTQYFGPGTRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 896 - protein, 7 aa, Homo sapiens
TISGTDY

SEQ ID NO: 897 - protein, 5 aa, Homo sapiens
GLTSN

SEQ ID NO: 898 - protein, 16 aa, Homo sapiens
ILRDWSAGGTSYGKLT

SEQ ID NO: 899 - protein, 122 aa, Homo sapiens
KTTQPNSMESNEEEPVHLPCNHSTISGTDYIHWYRQLPSQGPEYVIHGLTSNVNNRMASLAIAEDRKSSTLILHRATLRDAAVYYCILRDWSAGGTSYGKLTFGQGTILTVHPNIQNPEPAV

SEQ ID NO: 900 - protein, 141 aa, Homo sapiens
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILRDWSAGGTSYGKLTFGQGTILTVHPNIQNPEPAV

SEQ ID NO: 901 - protein, 278 aa, synthetic construct
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILRDWSAGGTSYGKLTFGQGTILTVHPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 902 - protein, 6 aa, Homo sapiens
DFQATT

SEQ ID NO: 903 - protein, 7 aa, Homo sapiens
SNEGSKA

SEQ ID NO: 904 - protein, 11 aa, Homo sapiens
SADQGVTYGYT

SEQ ID NO: 905 - protein, 119 aa, Homo sapiens
AVVSQHPSRVICKSGTSVKIECRSLDFQATTMFWYRQFPKQSLMLMATSNEGSKATYEQGVEKDKFLINHASLTLSTLTVTSAHPEDSSFYICSADQGVTYGYTFGSGTRLTVVEDLRN

SEQ ID NO: 906 - protein, 134 aa, Homo sapiens
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSADQGVTYGYTFGSGTRLTVVEDLRN

SEQ ID NO: 907 - protein, 307 aa, synthetic construct
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSADQGVTYGYTFGSGTRLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 908 - protein, 6 aa, Homo sapiens
SSVPPY

SEQ ID NO: 909 - protein, 8 aa, Homo sapiens
YTSAATLV

SEQ ID NO: 910 - protein, 13 aa, Homo sapiens
AVSRPTGTASKLT

SEQ ID NO: 911 - protein, 123 aa, Homo sapiens
QSVTQLGSHVSVSEGALVLLRCNYSSSVPPYLFWYVQYPNQGLQLLLKYTSAATLVKGINGFEAEFKKSETSFHLTKPSAHMSDAAEYFCAVSRPTGTASKLTFGTGTRLQVTLNIQNPEPAV

SEQ ID NO: 912 - protein, 143 aa, Homo sapiens
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSRPTGTASKLTFGTGTRLQVTLNIQNPEPAV

SEQ ID NO: 913 - protein, 280 aa, synthetic construct
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSRPTGTASKLTFGTGTRLQVTLNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 914 - protein, 6 aa, Homo sapiens
DFQATT

SEQ ID NO: 915 - protein, 7 aa, Homo sapiens
SNEGSKA

SEQ ID NO: 916 - protein, 11 aa, Homo sapiens
SARAPSDSEAF

SEQ ID NO: 917 - protein, 119 aa, Homo sapiens
AVVSQHPSWVICKSGTSVKIECRSLDFQATTMFWYRQFPKQSLMLMATSNEGSKATYEQGVEKDKFLINHASLTLSTLTVTSAHPEDSSFYICSARAPSDSEAFFGQGTRLTVVEDLRN

SEQ ID NO: 918 - protein, 134 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARAPSDSEAFFGQGTRLTVVEDLRN

SEQ ID NO: 919 - protein, 307 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARAPSDSEAFFGQGTRLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 920 - protein, 6 aa, Homo sapiens
NTAFDY

SEQ ID NO: 921 - protein, 7 aa, Homo sapiens
IRPDVSE

SEQ ID NO: 922 - protein, 14 aa, Homo sapiens
AASMRFSNTGNQFY

SEQ ID NO: 923 - protein, 131 aa, Homo sapiens
QQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASMRFSNTGNQFYFGTGTSLTVIPNIQNPEPAV

SEQ ID NO: 924 - protein, 152 aa, Homo sapiens
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASMRFSNTGNQFYFGTGTSLTVIPNIQNPEPAV

SEQ ID NO: 925 - protein, 289 aa, synthetic construct
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASMRFSNTGNQFYFGTGTSLTVIPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 926 - protein, 5 aa, Homo sapiens
ENHRY

SEQ ID NO: 927 - protein, 6 aa, Homo sapiens
SYGVKD

SEQ ID NO: 928 - protein, 11 aa, Homo sapiens
AISEWGGNTIY

SEQ ID NO: 929 - protein, 115 aa, Homo sapiens
GITQSPRHKVTETGTPVTLRCHQTENHRYMYWYRQDPGHGLRLIHYSYGVKDTDKGEVSDGYSVSRSKTEDFLLTLESATSSQTSVYFCAISEWGGNTIYFGEGSWLTVVEDLRN

SEQ ID NO: 930 - protein, 136 aa, Homo sapiens
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISEWGGNTIYFGEGSWLTVVEDLRN

SEQ ID NO: 931 - protein, 309 aa, synthetic construct
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISEWGGNTIYFGEGSWLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 932 - protein, 6 aa, Homo sapiens
NTAFDY

SEQ ID NO: 933 - protein, 7 aa, Homo sapiens
IRPDVSE

SEQ ID NO: 934 - protein, 11 aa, Homo sapiens
AASYRGGNQFY

SEQ ID NO: 935 - protein, 128 aa, Homo sapiens
QQKEKSDQQQVKQSPQSLIVQKGGIPIINCAYENTAFDYFPWYQQFPGKGPALLIAIRPDVSEKKEGRFTISFNKSAKQFSLHIMDSQPGDSATYFCAASYRGGNQFYFGTGTSLTVIPNIQNPEPAV

SEQ ID NO: 936 - protein, 149 aa, Homo sapiens
MDKILGASFLVLWLQLCWVSGQQKEKSDQQQVKQSPQSLIVQKGGIPIINCAYENTAFDYFPWYQQFPGKGPALLIAIRPDVSEKKEGRFTISFNKSAKQFSLHIMDSQPGDSATYFCAASYRGGNQFYFGTGTSLTVIPNIQNPEPAV

SEQ ID NO: 937 - protein, 286 aa, synthetic construct
MDKILGASFLVLWLQLCWVSGQQKEKSDQQQVKQSPQSLIVQKGGIPIINCAYENTAFDYFPWYQQFPGKGPALLIAIRPDVSEKKEGRFTISFNKSAKQFSLHIMDSQPGDSATYFCAASYRGGNQFYFGTGTSLTVIPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 938 - protein, 6 aa, Homo sapiens
DFQATT

SEQ ID NO: 939 - protein, 7 aa, Homo sapiens
SNEGSKA

SEQ ID NO: 940 - protein, 12 aa, Homo sapiens
SAFRTGDSEKLF

SEQ ID NO: 941 - protein, 120 aa, Homo sapiens
AVVSQHPSWVICKSGTSVKIECRSLDFQATTMFWYRQFPKQSLMLMATSNEGSKATYEQGVEKDKFLINHASLTLSTLTVTSAHPEDSSFYICSAFRTGDSEKLFFGSGTQLSVLEDLRN

SEQ ID NO: 942 - protein, 135 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAFRTGDSEKLFFGSGTQLSVLEDLRN

SEQ ID NO: 943 - protein, 308 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAFRTGDSEKLFFGSGTQLSVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 944 - protein, 6 aa, Homo sapiens
ATGYPS

SEQ ID NO: 945 - protein, 7 aa, Homo sapiens
ATKADDK

SEQ ID NO: 946 - protein, 14 aa, Homo sapiens
ALSETSGGGADGLT

SEQ ID NO: 947 - protein, 123 aa, Homo sapiens
NSVTQMEGPVTLSEEAFLTINCTYTATGYPSLFWYVQYPGEGLQLLLKATKADDKGSNKGFEATYRKETTSFHLEKGSVQVSDSAVYFCALSETSGGGADGLTFGKGTHLIIQPNIQNPEPAV

SEQ ID NO: 948 - protein, 143 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSETSGGGADGLTFGKGTHLIIQPNIQNPEPAV

SEQ ID NO: 949 - protein, 280 aa, synthetic construct
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSETSGGGADGLTFGKGTHLIIQPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 950 - protein, 6 aa, Homo sapiens
DFQATT

SEQ ID NO: 951 - protein, 7 aa, Homo sapiens
SNEGSKA

SEQ ID NO: 952 - protein, 12 aa, Homo sapiens
SAVGGIYHNEQF

SEQ ID NO: 953 - protein, 120 aa, Homo sapiens
AVVSQHPSWVICKSGTSVKIECRSLDFQATTMFWYRQFPKQSLMLMATSNEGSKATYEQGVEKDKFLINHASLTLSTLTVTSAHPEDSSFYICSAVGGIYHNEQFFGPGTRLTVLEDLRN

SEQ ID NO: 954 - protein, 135 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAVGGIYHNEQFFGPGTRLTVLEDLRN

SEQ ID NO: 955 - protein, 308 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAVGGIYHNEQFFGPGTRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 956 - protein, 6 aa, Homo sapiens
SSVPPY

SEQ ID NO: 957 - protein, 8 aa, Homo sapiens
YTTGATLV

SEQ ID NO: 958 - protein, 12 aa, Homo sapiens
AVTEPGGYQKVT

SEQ ID NO: 959 - protein, 122 aa, Homo sapiens
QSVTQLGSHVSVSEGALVLLRCNYSSSVPPYLFWYVQYPNQGLQLLLKYTTGATLVKGINGFEAEFKKSETSFHLTKPSAHMSDAAEYFCAVTEPGGYQKVTFGTGTKLQVIPNIQNPEPAV

SEQ ID NO: 960 - protein, 142 aa, Homo sapiens
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVTEPGGYQKVTFGTGTKLQVIPNIQNPEPAV

SEQ ID NO: 961 - protein, 279 aa, synthetic construct
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVTEPGGYQKVTFGTGTKLQVIPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 962 - protein, 6 aa, Homo sapiens
DFQATT

SEQ ID NO: 963 - protein, 7 aa, Homo sapiens
SNEGSKA

SEQ ID NO: 964 - protein, 13 aa, Homo sapiens
SATGQHAGANVLT

SEQ ID NO: 965 - protein, 121 aa, Homo sapiens
AVVSQHPSRVICKSGTSVKIECRSLDFQATTMFWYRQFPKQSLMLMATSNEGSKATYEQGVEKDKFLINHASLTLSTLTVTSAHPEDSSFYICSATGQHAGANVLTFGAGSRLTVLEDLRN

SEQ ID NO: 966 - protein, 136 aa, Homo sapiens
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSATGQHAGANVLTFGAGSRLTVLEDLRN

SEQ ID NO: 967 - protein, 309 aa, synthetic construct
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSATGQHAGANVLTFGAGSRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 968 - protein, 6 aa, Homo sapiens
SSVPPY

SEQ ID NO: 969 - protein, 8 aa, Homo sapiens
YTSAATLV

SEQ ID NO: 970 - protein, 12 aa, Homo sapiens
AVSERNNNARLM

SEQ ID NO: 971 - protein, 122 aa, Homo sapiens
QSVTQLGSHVSVSEGALVLLRCNYSSSVPPYLFWYVQYPNQGLQLLLKYTSAATLVKGINGFEAEFKKSETSFHLTKPSAHMSDAAEYFCAVSERNNNARLMFGDGTQLVVKPNIQNPEPAV

SEQ ID NO: 972 - protein, 142 aa, Homo sapiens
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSERNNNARLMFGDGTQLVVKPNIQNPEPAV

SEQ ID NO: 973 - protein, 279 aa, synthetic construct
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSERNNNARLMFGDGTQLVVKPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 974 - protein, 5 aa, Homo sapiens
SGHDT

SEQ ID NO: 975 - protein, 6 aa, Homo sapiens
YYEEEE

SEQ ID NO: 976 - protein, 11 aa, Homo sapiens
ASSHSGTYEQY

SEQ ID NO: 977 - protein, 115 aa, Homo sapiens
GVTQSPTHLIKTRGQQVTLRCSPKSGHDTVSWYQQALGQGPQFIFQYYEEEERQRGNFPDRFSGHQFPNYSSELNVNALLLGDSALYLCASSHSGTYEQYFGPGTRLTVTEDLRN

SEQ ID NO: 978 - protein, 136 aa, Homo sapiens
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSHSGTYEQYFGPGTRLTVTEDLRN

SEQ ID NO: 979 - protein, 309 aa, synthetic construct
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSHSGTYEQYFGPGTRLTVTEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 980 - protein, 6 aa, Homo sapiens
NSMFDY

SEQ ID NO: 981 - protein, 7 aa, Homo sapiens
ISSIKDK

SEQ ID NO: 982 - protein, 10 aa, Homo sapiens
AASVKTDKLI

SEQ ID NO: 983 - protein, 125 aa, Homo sapiens
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASVKTDKLIFGTGTRLQVFPNIQNPEPAV

SEQ ID NO: 984 - protein, 146 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASVKTDKLIFGTGTRLQVFPNIQNPEPAV

SEQ ID NO: 985 - protein, 283 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASVKTDKLIFGTGTRLQVFPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 986 - protein, 5 aa, Homo sapiens
ENHRY

SEQ ID NO: 987 - protein, 6 aa, Homo sapiens
SYGVKD

SEQ ID NO: 988 - protein, 17 aa, Homo sapiens
AIREPLGLAKSSYNEQF

SEQ ID NO: 989 - protein, 121 aa, Homo sapiens
GITQSPRHKVTETGTPVTLRCHQTENHRYMYWYRQDPGHGLRLIHYSYGVKDTDKGEVSDGYSVSRSKTEDFLLTLESATSSQTSVYFCAIREPLGLAKSSYNEQFFGPGTRLTVLEDLRN

SEQ ID NO: 990 - protein, 142 aa, Homo sapiens
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAIREPLGLAKSSYNEQFFGPGTRLTVLEDLRN

SEQ ID NO: 991 - protein, 315 aa, synthetic construct
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAIREPLGLAKSSYNEQFFGPGTRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 992 - protein, 6 aa, Homo sapiens
SSVPPY

SEQ ID NO: 993 - protein, 8 aa, Homo sapiens
YTTGATLV

SEQ ID NO: 994 - protein, 12 aa, Homo sapiens
AVSGRNNNARLM

SEQ ID NO: 995 - protein, 122 aa, Homo sapiens
QSVTQLGSHVSVSEGALVLLRCNYSSSVPPYLFWYVQYPNQGLQLLLKYTTGATLVKGINGFEAEFKKSETSFHLTKPSAHMSDAAEYFCAVSGRNNNARLMFGDGTQLVVKPNIQNPEPAV

SEQ ID NO: 996 - protein, 142 aa, Homo sapiens
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVSGRNNNARLMFGDGTQLVVKPNIQNPEPAV

SEQ ID NO: 997 - protein, 279 aa, synthetic construct
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVSGRNNNARLMFGDGTQLVVKPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 998 - protein, 5 aa, Homo sapiens
SGHDT

SEQ ID NO: 999 - protein, 6 aa, Homo sapiens
YYEEEE

SEQ ID NO: 1000 - protein, 11 aa, Homo sapiens
ASRRQGSYEQY

SEQ ID NO: 1001 - protein, 115 aa, Homo sapiens
GVTQSPTHLIKTRGQQVTLRCSPKSGHDTVSWYQQALGQGPQFIFQYYEEEERQRGNFPDRFSGHQFPNYSSELNVNALLLGDSALYLCASRRQGSYEQYFGPGTRLTVTEDLRN

SEQ ID NO: 1002 - protein, 136 aa, Homo sapiens
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASRRQGSYEQYFGPGTRLTVTEDLRN

SEQ ID NO: 1003 - protein, 309 aa, synthetic construct
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASRRQGSYEQYFGPGTRLTVTEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1004 - protein, 137 aa, Mus musculus
  VARIANT 1: note = X is N, D, H, or Y
  VARIANT 48: note = X is T or C
  VARIANT 112: note = X is S, A, V, L, I, P, F, M, or W
  VARIANT 114: note = X is M, A, V, L, I, P, F, or W
  VARIANT 115: note = X is G, A, V, L, I, P, F, M, or W
XIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 1005 - DNA, 411 bp, Mus musculus
  source 1..411: mol_type = other DNA
  misc_feature 1..3: note = NNN make up a codon that encodes N, D, H, or Y
  misc_feature 142..144: note = NNN make up a codon that encodes T or C
  misc_feature 334..336: note = NNN make up a codon that encodes S, A, V, L, I, P, F, M, or W
  misc_feature 340..342: note = NNN make up a codon that encodes M, A, V, L, I, P, F, or W
  misc_feature 343..345: note = NNN make up a codon that encodes G, A, V, L, I, P, F, M, or W
nnnatccagaatcccgagcctgcggtgtaccagctgaaggacccccgctctcaggatagcacactgtgcctgttcaccgactttgatagccagatcaacgtgcctaaaacaatggagtccggcaccttcatcaccgacaagnnngtgctggatatgaaagcgatggactccaagtctaacggcgcgatcgcgtggtccaatcagacatctttcacctgccaggatatcttcaaggagacaaacgcgacctatccttcctctgacgtgccatgtgatgcgacactgaccgagaagagcttcgagacagacatgaacctgaattttcagaatctgnnngtcnnnnnnctgagaatcctgctgctgaaggtggcgggctttaatctgctgatgacactgcggctgtggagttcc

SEQ ID NO: 1006 - protein, 137 aa, synthetic construct
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1007 - DNA, 411 bp, synthetic construct
  source 1..411: mol_type = other DNA
aacatccagaatcccgagcctgcggtgtaccagctgaaggacccccgctctcaggatagcacactgtgcctgttcaccgactttgatagccagatcaacgtgcctaaaacaatggagtccggcaccttcatcaccgacaagtgcgtgctggatatgaaagcgatggactccaagtctaacggcgcgatcgcgtggtccaatcagacatctttcacctgccaggatatcttcaaggagacaaacgcgacctatccttcctctgacgtgccatgtgatgcgacactgaccgagaagagcttcgagacagacatgaacctgaattttcagaatctgctggtcatcgtgctgagaatcctgctgctgaaggtggcgggctttaatctgctgatgacactgcggctgtggagttcc

SEQ ID NO: 1008 - protein, 137 aa, synthetic construct
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1009 - DNA, 411 bp, synthetic construct
  source 1..411: mol_type = other DNA
aacatccagaatcccgagcctgcggtgtaccagctgaaggacccccgctctcaggatagcacactgtgcctgttcaccgactttgatagccagatcaacgtgcctaaaacaatggagtccggcaccttcatcaccgacaagaccgtgctggatatgaaagcgatggactccaagtctaacggcgcgatcgcgtggtccaatcagacatctttcacctgccaggatatcttcaaggagacaaacgcgacctatccttcctctgacgtgccatgtgatgcgacactgaccgagaagagcttcgagacagacatgaacctgaattttcagaatctgctggtcatcgtgctgagaatcctgctgctgaaggtggcgggctttaatctgctgatgacactgcggctgtggagttcc

SEQ ID NO: 1010 - protein, 137 aa, synthetic construct
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 1011 - protein, 137 aa, Mus musculus
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 1012 - protein, 141 aa, Homo sapiens
  VARIANT 1: note = X is N, D, H, or Y
  VARIANT 48: note = X is T or C
  VARIANT 116: note = X is S, A, V, L, I, P, F, M, or W
  VARIANT 118: note = X is M, A, V, L, I, P, F, or W
  VARIANT 119: note = X is G, A, V, L, I, P, F, M, or W
XIQNPDPAVYQLRDSKSSDKSVCLFTDFDSQTNVSQSKDSDVYITDKXVLDMRSMDFKSNSAVAWSNKSDFACANAFNNSIIPEDTFFPSPESSCDVKLVEKSFETDTNLNFQNLXVXXFRILLLKVAGFNLLMTLRLWSS

SEQ ID NO: 1013 - protein, 141 aa, synthetic construct
  VARIANT 1: note = X is N, D, H, or Y
XIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVFRILLLKVAGFNLLMTLRLWSS

SEQ ID NO: 1014 - protein, 141 aa, synthetic construct
  VARIANT 1: note = X is N, D, H, or Y
XIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLLVIVFRILLLKVAGFNLLMTLRLWSS

SEQ ID NO: 1015 - protein, 141 aa, synthetic construct
  VARIANT 1: note = X is N, D, H, or Y
XIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 1016 - protein, 141 aa, Homo sapiens
  VARIANT 1: note = X is N, D, H, or Y
XIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 1017 - protein, 173 aa, Mus musculus
  VARIANT 57: note = X is S or C
EDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1018 - DNA, 519 bp, Mus musculus
  source 1..519: mol_type = other DNA
  misc_feature 169..171: note = NNN make up a codon that encodes S or C
gaggacctgaggaacgtgaccccacctaaagtgagcctgttcgagccatccaaggcggagatcgcgaataagcagaaagcgaccctggtgtgcctggcgaggggcttctttcccgatcacgtggagctgtcctggtgggtgaacggcaaagaggtgcactctggcgtgnnnacagaccctcaggcgtacaaggagagcaattactcctattgtctgtctagcagactgagggtgagcgcgaccttttggcacaacccccggaatcacttccgctgccaggtgcagtttcacggcctgtccgaggaggataaatggcctgagggctctccaaagcccgtgacacagaatatcagcgcggaggcgtggggaagagcggactgtggcattacaagcgcgtcctatcagcagggcgtgctgtccgcgaccatcctgtacgagattctgctgggcaaggcgacactgtatgcggtgctggtgtccaccctggtggtcatggcgatggtgaagaggaaaaactct

SEQ ID NO: 1019 - protein, 173 aa, synthetic construct
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1020 - DNA, 519 bp, synthetic construct
  source 1..519: mol_type = other DNA
gaggacctgaggaacgtgaccccacctaaagtgagcctgttcgagccatccaaggcggagatcgcgaataagcagaaagcgaccctggtgtgcctggcgaggggcttctttcccgatcacgtggagctgtcctggtgggtgaacggcaaagaggtgcactctggcgtgtgcacagaccctcaggcgtacaaggagagcaattactcctattgtctgtctagcagactgagggtgagcgcgaccttttggcacaacccccggaatcacttccgctgccaggtgcagtttcacggcctgtccgaggaggataaatggcctgagggctctccaaagcccgtgacacagaatatcagcgcggaggcgtggggaagagcggactgtggcattacaagcgcgtcctatcagcagggcgtgctgtccgcgaccatcctgtacgagattctgctgggcaaggcgacactgtatgcggtgctggtgtccaccctggtggtcatggcgatggtgaagaggaaaaactct

SEQ ID NO: 1021 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1022 - protein, 179 aa, Homo sapiens
  VARIANT 57: note = X is S or C
EDLKNVFPPEVAVFEPSEAEISHTQKATLVCLATGFYPDHVELSWWVNGKEVHSGVXTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 1023 - protein, 179 aa, synthetic construct
EDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 1024 - protein, 179 aa, Homo sapiens
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 1025 - protein, 6 aa, Homo sapiens
NSMFDY

SEQ ID NO: 1026 - protein, 7 aa, Homo sapiens
ISSIKDK

SEQ ID NO: 1027 - protein, 11 aa, Homo sapiens
AARKLQGGKLI

SEQ ID NO: 1028 - protein, 126 aa, Homo sapiens
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAARKLQGGKLIFGQGTELSVKPNIQNPEPAV

SEQ ID NO: 1029 - protein, 147 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAARKLQGGKLIFGQGTELSVKPNIQNPEPAV

SEQ ID NO: 1030 - protein, 284 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAARKLQGGKLIFGQGTELSVKPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1031 - protein, 5 aa, Homo sapiens
SGHVS

SEQ ID NO: 1032 - protein, 6 aa, Homo sapiens
FNYEAQ

SEQ ID NO: 1033 - protein, 17 aa, Homo sapiens
ASSLWLGALSSGANVLT

SEQ ID NO: 1034 - protein, 122 aa, Homo sapiens
GVSQSPRYKVTKRGQDVALRCDPISGHVSLYWYRQALGQGPEFLTYFNYEAQQDKSGLPNDRFSAERPEGSISTLTIQRTEQRDSAMYRCASSLWLGALSSGANVLTFGAGSRLTVLEDLRN

SEQ ID NO: 1035 - protein, 143 aa, Homo sapiens
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSLWLGALSSGANVLTFGAGSRLTVLEDLRN

SEQ ID NO: 1036 - protein, 316 aa, synthetic construct
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSLWLGALSSGANVLTFGAGSRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1037 - protein, 6 aa, Homo sapiens
NSAFQY

SEQ ID NO: 1038 - protein, 6 aa, Homo sapiens
TYSSGN

SEQ ID NO: 1039 - protein, 12 aa, Homo sapiens
AMSYNTNAGKST

SEQ ID NO: 1040 - protein, 122 aa, Homo sapiens
QQKEVEQDPGPLSVPEGAIVSLNCTYSNSAFQYFMWYRQYSRKGPELLMYTYSSGNKEDGRFTAQVDKSSKYISLFIRDSQPSDSATYLCAMSYNTNAGKSTFGDGTTLTVKPNIQNPEPAV

SEQ ID NO: 1041 - protein, 143 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSYNTNAGKSTFGDGTTLTVKPNIQNPEPAV

SEQ ID NO: 1042 - protein, 280 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSYNTNAGKSTFGDGTTLTVKPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1043 - protein, 5 aa, Homo sapiens
SGHVS

SEQ ID NO: 1044 - protein, 6 aa, Homo sapiens
FQNEAQ

SEQ ID NO: 1045 - protein, 12 aa, Homo sapiens
ASSVRDRANEQF

SEQ ID NO: 1046 - protein, 117 aa, Homo sapiens
GVSQSPRYKVAKRGQDVALRCDPISGHVSLFWYQQALGQGPEFLTYFQNEAQLDKSGLPSDRFFAERPEGSVSTLKIQRTQQEDSAVYLCASSVRDRANEQFFGPGTRLTVLEDLRN

SEQ ID NO: 1047 - protein, 138 aa, Homo sapiens
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSVRDRANEQFFGPGTRLTVLEDLRN

SEQ ID NO: 1048 - protein, 311 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSVRDRANEQFFGPGTRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1049 - protein, 6 aa, Homo sapiens
TSGFYG

SEQ ID NO: 1050 - protein, 6 aa, Homo sapiens
NALDGL

SEQ ID NO: 1051 - protein, 11 aa, Homo sapiens
AVPSSNTGKLI

SEQ ID NO: 1052 - protein, 118 aa, Homo sapiens
QSLEQPSEVTAVEGAIVQINCTYQTSGFYGLSWYQQHDGGAPTFLSYNALDGLEETGRFSSFLSRSDSYGYLLLQELQMKDSASYFCAVPSSNTGKLIFGQGTTLQVKPNIQNPEPAV

SEQ ID NO: 1053 - protein, 136 aa, Homo sapiens
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVPSSNTGKLIFGQGTTLQVKPNIQNPEPAV

SEQ ID NO: 1054 - protein, 273 aa, synthetic construct
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVPSSNTGKLIFGQGTTLQVKPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1055 - protein, 5 aa, Homo sapiens
SGHRS

SEQ ID NO: 1056 - protein, 6 aa, Homo sapiens
YFSETQ

SEQ ID NO: 1057 - protein, 11 aa, Homo sapiens
ASSLRTGEKLF

SEQ ID NO: 1058 - protein, 115 aa, Homo sapiens
GVTQTPRYLIKTRGQQVTLSCSPISGHRSVSWYQQTPGQGLQFLFEYFSETQRNKGNFPGRFSGRQFSNSRSEMNVSTLELGDSALYLCASSLRTGEKLFFGSGTQLSVLEDLRN

SEQ ID NO: 1059 - protein, 136 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLRTGEKLFFGSGTQLSVLEDLRN

SEQ ID NO: 1060 - protein, 309 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLRTGEKLFFGSGTQLSVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1061 - protein, 6 aa, Homo sapiens
NSMFDY

SEQ ID NO: 1062 - protein, 7 aa, Homo sapiens
ISSIKDK

SEQ ID NO: 1063 - protein, 11 aa, Homo sapiens
AASALTGNQFY

SEQ ID NO: 1064 - protein, 126 aa, Homo sapiens
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASALTGNQFYFGTGTSLTVIPNIQNPEPAV

SEQ ID NO: 1065 - protein, 147 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASALTGNQFYFGTGTSLTVIPNIQNPEPAV

SEQ ID NO: 1066 - protein, 284 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASALTGNQFYFGTGTSLTVIPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1067 - protein, 5 aa, Homo sapiens
SGHAT

SEQ ID NO: 1068 - protein, 6 aa, Homo sapiens
FQNNGV

SEQ ID NO: 1069 - protein, 12 aa, Homo sapiens
ASSTGTGPRPQH

SEQ ID NO: 1070 - protein, 117 aa, Homo sapiens
GVAQSPRYKIIEKRQSVAFWCNPISGHATLYWYQQILGQGPKLLIQFQNNGVVDDSQLPKDRFSAERLKGVDSTLKIQPAKLEDSAVYLCASSTGTGPRPQHFGDGTRLSILEDLRN

SEQ ID NO: 1071 - protein, 138 aa, Homo sapiens
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSTGTGPRPQHFGDGTRLSILEDLRN

SEQ ID NO: 1072 - protein, 311 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSTGTGPRPQHFGDGTRLSILEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1073 - protein, 7 aa, Homo sapiens
TSESDYY

SEQ ID NO: 1074 - protein, 8 aa, Homo sapiens
QEAYKQQN

SEQ ID NO: 1075 - protein, 9 aa, Homo sapiens
AFSGNTPLV

SEQ ID NO: 1076 - protein, 120 aa, Homo sapiens
QTVTQSQPEMSVQEAETVTLSCTYDTSESDYYLFWYKQPPSRQMILVIRQEAYKQQNATENRFSVNFQKAAKSFSLKISDSQLGDAAMYFCAFSGNTPLVFGKGTRLSVIANIQNPEPAV

SEQ ID NO: 1077 - protein, 141 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAFSGNTPLVFGKGTRLSVIANIQNPEPAV

SEQ ID NO: 1078 - protein, 278 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAFSGNTPLVFGKGTRLSVIANIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1079 - protein, 5 aa, Homo sapiens
SEHNR

SEQ ID NO: 1080 - protein, 6 aa, Homo sapiens
FQNEAQ

SEQ ID NO: 1081 - protein, 15 aa, Homo sapiens
ASSSLRTSGTYNEQF

SEQ ID NO: 1082 - protein, 122 aa, Homo sapiens
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSSLRTSGTYNEQFFGPGTRLTVLEDLRN

SEQ ID NO: 1083 - protein, 141 aa, Homo sapiens
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSSLRTSGTYNEQFFGPGTRLTVLEDLRN

SEQ ID NO: 1084 - protein, 314 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSSLRTSGTYNEQFFGPGTRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1085 - protein, 6 aa, Homo sapiens
DSSSTY

SEQ ID NO: 1086 - protein, 7 aa, Homo sapiens
IFSNMDM

SEQ ID NO: 1087 - protein, 13 aa, Homo sapiens
AESMGSDSWGKFQ

SEQ ID NO: 1088 - protein, 121 aa, Homo sapiens
EDVEQSLFLSVREGDSSVINCTYTDSSSTYLYWYKQEPGAGLQLLTYIFSNMDMKQDQRLTVLLNKKDKHLSLRIADTQTGDSAIYFCAESMGSDSWGKFQFGAGTQVVVTPNIQNPEPAV

SEQ ID NO: 1089 - protein, 143 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESMGSDSWGKFQFGAGTQVVVTPNIQNPEPAV

SEQ ID NO: 1090 - protein, 280 aa, synthetic construct
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESMGSDSWGKFQFGAGTQVVVTPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1091 - protein, 5 aa, Homo sapiens
MNHNS

SEQ ID NO: 1092 - protein, 6 aa, Homo sapiens
SASEGT

SEQ ID NO: 1093 - protein, 13 aa, Homo sapiens
ASTPGQFSYNEQF

SEQ ID NO: 1094 - protein, 117 aa, Homo sapiens
GVTQTPKFQVLKTGQSMTLQCAQDMNHNSMYWYRQDPGMGLRLIYYSASEGTTDKGEVPNGYNVSRLNKREFSLRLESAAPSQTSVYFCASTPGQFSYNEQFFGPGTRLTVLEDLRN

SEQ ID NO: 1095 - protein, 138 aa, Homo sapiens
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASTPGQFSYNEQFFGPGTRLTVLEDLRN

SEQ ID NO: 1096 - protein, 311 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASTPGQFSYNEQFFGPGTRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1097 - protein, 6 aa, Homo sapiens
NSAFQY

SEQ ID NO: 1098 - protein, 6 aa, Homo sapiens
TYSSGN

SEQ ID NO: 1099 - protein, 11 aa, Homo sapiens
AMRGDSSYKLI

SEQ ID NO: 1100 - protein, 121 aa, Homo sapiens
QQKEVEQDPGPLSVPEGAIVSLNCTYSNSAFQYFMWYRQYSRKGPELLMYTYSSGNKEDGRFTAQVDKSSKYISLFIRDSQPSDSATYLCAMRGDSSYKLIFGSGTRLLVRPNIQNPEPAV

SEQ ID NO: 1101 - protein, 142 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMRGDSSYKLIFGSGTRLLVRPNIQNPEPAV

SEQ ID NO: 1102 - protein, 279 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMRGDSSYKLIFGSGTRLLVRPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1103 - protein, 5 aa, Homo sapiens
LNHDA

SEQ ID NO: 1104 - protein, 6 aa, Homo sapiens
SQIVND

SEQ ID NO: 1105 - protein, 14 aa, Homo sapiens
ASTQFQGKGNQPQH

SEQ ID NO: 1106 - protein, 118 aa, Homo sapiens
GITQSPKYLFRKEGQNVTLSCEQNLNHDAMYWYRQDPGQGLRLIYYSQIVNDFQKGDIAEGYSVSREKKESFPLTVTSAQKNPTAFYLCASTQFQGKGNQPQHFGDGTRLSILEDLRN

SEQ ID NO: 1107 - protein, 139 aa, Homo sapiens
MDTRLLCCAVICLLGADTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASTQFQGKGNQPQHFGDGTRLSILEDLRN

SEQ ID NO: 1108 - protein, 312 aa, synthetic construct
MDTRLLCCAVICLLGADTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASTQFQGKGNQPQHFGDGTRLSILEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1109 - protein, 5 aa, Homo sapiens
SIFNT

SEQ ID NO: 1110 - protein, 7 aa, Homo sapiens
LYKAGEL

SEQ ID NO: 1111 - protein, 13 aa, Homo sapiens
AGRHGSSNTGKLI

SEQ ID NO: 1112 - protein, 121 aa, Homo sapiens
QQLNQSPQSMFIQEGEDVSMNCTSSSIFNTWLWYKQDPGEGPVLLIALYKAGELTSNGRLTAQFGITRKDSFLNISASIPSDVGIYFCAGRHGSSNTGKLIFGQGTTLQVKPNIQNPEPAV

SEQ ID NO: 1113 - protein, 140 aa, Homo sapiens
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGRHGSSNTGKLIFGQGTTLQVKPNIQNPEPAV

SEQ ID NO: 1114 - protein, 277 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGRHGSSNTGKLIFGQGTTLQVKPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1115 - protein, 5 aa, Homo sapiens
LNHDA

SEQ ID NO: 1116 - protein, 6 aa, Homo sapiens
SQIVND

SEQ ID NO: 1117 - protein, 11 aa, Homo sapiens
ASSITGGYEQY

SEQ ID NO: 1118 - protein, 115 aa, Homo sapiens
GITQSPKYLFRKEGQNVTLSCEQNLNHDAMYWYRQDPGQGLRLIYYSQIVNDFQKGDIAEGYSVSREKKESFPLTVTSAQKNPTAFYLCASSITGGYEQYFGPGTRLTVTEDLRN

SEQ ID NO: 1119 - protein, 136 aa, Homo sapiens
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSITGGYEQYFGPGTRLTVTEDLRN

SEQ ID NO: 1120 - protein, 309 aa, synthetic construct
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSITGGYEQYFGPGTRLTVTEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1121 - protein, 6 aa, Homo sapiens
NSMFDY

SEQ ID NO: 1122 - protein, 7 aa, Homo sapiens
ISSIKDK

SEQ ID NO: 1123 - protein, 12 aa, Homo sapiens
AALSSGSARQLT

SEQ ID NO: 1124 - protein, 127 aa, Homo sapiens
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAALSSGSARQLTFGSGTQLTVLPNIQNPEPAV

SEQ ID NO: 1125 - protein, 148 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAALSSGSARQLTFGSGTQLTVLPNIQNPEPAV

SEQ ID NO: 1126 - protein, 285 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAALSSGSARQLTFGSGTQLTVLPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1127 - protein, 5 aa, Homo sapiens
SGHRS

SEQ ID NO: 1128 - protein, 6 aa, Homo sapiens
YFSETQ

SEQ ID NO: 1129 - protein, 13 aa, Homo sapiens
ASSVSLGDTGELF

SEQ ID NO: 1130 - protein, 117 aa, Homo sapiens
GVTQTPRYLIKTRGQQVTLSCSPISGHRSVSWYQQTPGQGLQFLFEYFSETQRNKGNFPGRFSGRQFSNSRSEMNVSTLELGDSALYLCASSVSLGDTGELFFGEGSRLTVLEDLRN

SEQ ID NO: 1131 - protein, 138 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSVSLGDTGELFFGEGSRLTVLEDLRN

SEQ ID NO: 1132 - protein, 311 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSVSLGDTGELFFGEGSRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1133 - protein, 5 aa, Homo sapiens
KALYS

SEQ ID NO: 1134 - protein, 7 aa, Homo sapiens
LLKGGEQ

SEQ ID NO: 1135 - protein, 12 aa, Homo sapiens
GTNGVGGADGLT

SEQ ID NO: 1136 - protein, 120 aa, Homo sapiens
QQPVQSPQAVILREGEDAVINCSSSKALYSVHWYRQKHGEAPVFLMILLKGGEQKGHEKISASFNEKKQQSSLYLTASQLSYSGTYFCGTNGVGGADGLTFGKGTHLIIQPNIQNPEPAV

SEQ ID NO: 1137 - protein, 141 aa, Homo sapiens
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTNGVGGADGLTFGKGTHLIIQPNIQNPEPAV

SEQ ID NO: 1138 - protein, 278 aa, synthetic construct
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTNGVGGADGLTFGKGTHLIIQPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1139 - protein, 5 aa, Homo sapiens
WNHNN

SEQ ID NO: 1140 - protein, 6 aa, Homo sapiens
SYGVHD

SEQ ID NO: 1141 - protein, 15 aa, Homo sapiens
ASSESSNWGMNTEAF

SEQ ID NO: 1142 - protein, 119 aa, Homo sapiens
EITQSPRHKITETGRQVTLACHQTWNHNNMFWYRQDLGHGLRLIHYSYGVHDTNKGEVSDGYSVSRSNTEDLPLTLESAASSQTSVYFCASSESSNWGMNTEAFFGQGTRLTVVEDLRN

SEQ ID NO: 1143 - protein, 140 aa, Homo sapiens
MGTRLFFYVALCLLWAGHRDAEITQSPRHKITETGRQVTLACHQTWNHNNMFWYRQDLGHGLRLIHYSYGVHDTNKGEVSDGYSVSRSNTEDLPLTLESAASSQTSVYFCASSESSNWGMNTEAFFGQGTRLTVVEDLRN

SEQ ID NO: 1144 - protein, 313 aa, synthetic construct
MGTRLFFYVALCLLWAGHRDAEITQSPRHKITETGRQVTLACHQTWNHNNMFWYRQDLGHGLRLIHYSYGVHDTNKGEVSDGYSVSRSNTEDLPLTLESAASSQTSVYFCASSESSNWGMNTEAFFGQGTRLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1145 - protein, 6 aa, Homo sapiens
NSASDY

SEQ ID NO: 1146 - protein, 7 aa, Homo sapiens
IRSNMDK

SEQ ID NO: 1147 - protein, 10 aa, Homo sapiens
AEISGTYKYI

SEQ ID NO: 1148 - protein, 119 aa, Homo sapiens
ENVGLHLPTLSVQEGDNSIINCAYSNSASDYFIWYKQESGKGPQFIIDIRSNMDKRQGQRVTVLLNKTVKHLSLQIAATQPGDSAVYFCAEISGTYKYIFGTGTRLKVLANIQNPEPAV

SEQ ID NO: 1149 - protein, 140 aa, Homo sapiens
MAGIRALFMYLWLQLDWVSRGENVGLHLPTLSVQEGDNSIINCAYSNSASDYFIWYKQESGKGPQFIIDIRSNMDKRQGQRVTVLLNKTVKHLSLQIAATQPGDSAVYFCAEISGTYKYIFGTGTRLKVLANIQNPEPAV

SEQ ID NO: 1150 - protein, 277 aa, synthetic construct
MAGIRALFMYLWLQLDWVSRGENVGLHLPTLSVQEGDNSIINCAYSNSASDYFIWYKQESGKGPQFIIDIRSNMDKRQGQRVTVLLNKTVKHLSLQIAATQPGDSAVYFCAEISGTYKYIFGTGTRLKVLANIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1151 - protein, 5 aa, Homo sapiens
SQVTM

SEQ ID NO: 1152 - protein, 7 aa, Homo sapiens
ANQGSEA

SEQ ID NO: 1153 - protein, 11 aa, Homo sapiens
SVAAGTNYGYT

SEQ ID NO: 1154 - protein, 118 aa, Homo sapiens
AVISQKPSRDICQRGTSLTIQCQVDSQVTMMFWYRQQPGQSLTLIATANQGSEATYESGFVIDKFPISRPNLTFSTLTVSNMSPEDSSIYLCSVAAGTNYGYTFGSGTRLTVVEDLRN

SEQ ID NO: 1155 - protein, 134 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVAAGTNYGYTFGSGTRLTVVEDLRN

SEQ ID NO: 1156 - protein, 307 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVAAGTNYGYTFGSGTRLTVVEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1157 - protein, 7 aa, Homo sapiens
TSENNYY

SEQ ID NO: 1158 - protein, 8 aa, Homo sapiens
QEAYKQQN

SEQ ID NO: 1159 - protein, 14 aa, Homo sapiens
AFNSFSGAGSYQLT

SEQ ID NO: 1160 - protein, 125 aa, Homo sapiens
QTVTQSQPEMSVQEAETVTLSCTYDTSENNYYLFWYKQPPSRQMILVIRQEAYKQQNATENRFSVNFQKAAKSFSLKISDSQLGDTAMYFCAFNSFSGAGSYQLTFGKGTKLSVIPNIQNPEPAV

SEQ ID NO: 1161 - protein, 146 aa, Homo sapiens
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFNSFSGAGSYQLTFGKGTKLSVIPNIQNPEPAV

SEQ ID NO: 1162 - protein, 283 aa, synthetic construct
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFNSFSGAGSYQLTFGKGTKLSVIPNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1163 - protein, 5 aa, Homo sapiens
SGHNT

SEQ ID NO: 1164 - protein, 6 aa, Homo sapiens
YYREEE

SEQ ID NO: 1165 - protein, 10 aa, Homo sapiens
ASSSLQETQY

SEQ ID NO: 1166 - protein, 114 aa, Homo sapiens
GVTQSPTHLIKTRGQQVTLRCSSQSGHNTVSWYQQALGQGPQFIFQYYREEENGRGNFPPRFSGLQFPNYSSELNVNALELDDSALYLCASSSLQETQYFGPGTRLLVLEDLRN

SEQ ID NO: 1167 - protein, 135 aa, Homo sapiens
MGPGLLCWALLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSSLQETQYFGPGTRLLVLEDLRN

SEQ ID NO: 1168 - protein, 308 aa, synthetic construct
MGPGLLCWALLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSSLQETQYFGPGTRLLVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1169 - protein, 7 aa, Homo sapiens
TSENNYY

SEQ ID NO: 1170 - protein, 8 aa, Homo sapiens
QEAYKQQN

SEQ ID NO: 1171 - protein, 13 aa, Homo sapiens
AFMKYTGGGNKLT

SEQ ID NO: 1172 - protein, 124 aa, Homo sapiens
QTVTQSQPEMSVQEAETVTLSCTYDTSENNYYLFWYKQPPSRQMILVIRQEAYKQQNATENRFSVNFQKAAKSFSLKISDSQLGDTAMYFCAFMKYTGGGNKLTFGTGTQLKVELNIQNPEPAV

SEQ ID NO: 1173 - protein, 145 aa, Homo sapiens
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMKYTGGGNKLTFGTGTQLKVELNIQNPEPAV

SEQ ID NO: 1174 - protein, 282 aa, synthetic construct
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMKYTGGGNKLTFGTGTQLKVELNIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1175 - protein, 5 aa, Homo sapiens
SNHLY

SEQ ID NO: 1176 - protein, 6 aa, Homo sapiens
FYNNEI

SEQ ID NO: 1177 - protein, 14 aa, Homo sapiens
ASSGTLAGEVDTQY

SEQ ID NO: 1178 - protein, 121 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSGTLAGEVDTQYFGPGTRLTVLEDLRN

SEQ ID NO: 1179 - protein, 140 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGTLAGEVDTQYFGPGTRLTVLEDLRN

SEQ ID NO: 1180 - protein, 313 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGTLAGEVDTQYFGPGTRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1181 - protein, 7 aa, Homo sapiens
TRDTTYY

SEQ ID NO: 1182 - protein, 8 aa, Homo sapiens
RNSFDEQN

SEQ ID NO: 1183 - protein, 13 aa, Homo sapiens
ALSELNTGGFKTI

SEQ ID NO: 1184 - protein, 124 aa, Homo sapiens
QKVTQAQTEISVVEKEDVTLDCVYETRDTTYYLFWYKQPPSGELVFLIRRNSFDEQNEISGRYSWNFQKSTSSFNFTITASQVVDSAVYFCALSELNTGGFKTIFGAGTRLFVKANIQNPEPAV

SEQ ID NO: 1185 - protein, 145 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSELNTGGFKTIFGAGTRLFVKANIQNPEPAV

SEQ ID NO: 1186 - protein, 282 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSELNTGGFKTIFGAGTRLFVKANIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1187 - protein, 5 aa, Homo sapiens
SGHAT

SEQ ID NO: 1188 - protein, 6 aa, Homo sapiens
FQDESV

SEQ ID NO: 1189 - protein, 14 aa, Homo sapiens
ASSISGTGGLDTQY

SEQ ID NO: 1190 - protein, 119 aa, Homo sapiens
EVAQSPRYKITEKSQAVAFWCDPISGHATLYWYRQILGQGPELLVQFQDESVVDDSQLPKDRFSAERLKGVDSTLKIQPAELGDSAMYLCASSISGTGGLDTQYFGPGTRLTVLEDLRN

SEQ ID NO: 1191 - protein, 140 aa, Homo sapiens
MSTRLLCWMALCLLGAELSEAEVAQSPRYKITEKSQAVAFWCDPISGHATLYWYRQILGQGPELLVQFQDESVVDDSQLPKDRFSAERLKGVDSTLKIQPAELGDSAMYLCASSISGTGGLDTQYFGPGTRLTVLEDLRN

SEQ ID NO: 1192 - protein, 313 aa, synthetic construct
MSTRLLCWMALCLLGAELSEAEVAQSPRYKITEKSQAVAFWCDPISGHATLYWYRQILGQGPELLVQFQDESVVDDSQLPKDRFSAERLKGVDSTLKIQPAELGDSAMYLCASSISGTGGLDTQYFGPGTRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 1193 - protein, 7 aa, Homo sapiens
TSDPSYG

SEQ ID NO: 1194 - protein, 8 aa, Homo sapiens
QGSYDQQN

SEQ ID NO: 1195 - protein, 13 aa, Homo sapiens
AMRDPGTGGFKTI

SEQ ID NO: 1196 - protein, 124 aa, Homo sapiens
QKITQTQPGMFVQEKEAVTLDCTYDTSDPSYGLFWYKQPSSGEMIFLIYQGSYDQQNATEGRYSLNFQKARKSANLVISASQLGDSAMYFCAMRDPGTGGFKTIFGAGTRLFVKANIQNPEPAV

SEQ ID NO: 1197 - protein, 145 aa, Homo sapiens
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMRDPGTGGFKTIFGAGTRLFVKANIQNPEPAV

SEQ ID NO: 1198 - protein, 282 aa, synthetic construct
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMRDPGTGGFKTIFGAGTRLFVKANIQNPEPAVNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 1199 - protein, 5 aa, Homo sapiens
SGHTA

SEQ ID NO: 1200 - protein, 6 aa, Homo sapiens
FQGNSA

SEQ ID NO: 1201 - protein, 16 aa, Homo sapiens
ASSSMATGGVVGDTQY

SEQ ID NO: 1202 - protein, 120 aa, Homo sapiens
VSQSPSNKVTEKGKDVELRCDPISGHTALYWYRQRLGQGLEFLIYFQGNSAPDKSGLPSDRFSAERTGESVSTLTIQRTQQEDSAVYLCASSSMATGGVVGDTQYFGPGTRLTVLEDLRN

SEQ ID NO: 1203 - protein, 142 aa, Homo sapiens
MGTRLLFWVAFCLLGAYHTGAGVSQSPSNKVTEKGKDVELRCDPISGHTALYWYRQRLGQGLEFLIYFQGNSAPDKSGLPSDRFSAERTGESVSTLTIQRTQQEDSAVYLCASSSMATGGVVGDTQYFGPGTRLTVLEDLRN

SEQ ID NO: 1204 - protein, 315 aa, synthetic construct
MGTRLLFWVAFCLLGAYHTGAGVSQSPSNKVTEKGKDVELRCDPISGHTALYWYRQRLGQGLEFLIYFQGNSAPDKSGLPSDRFSAERTGESVSTLTIQRTQQEDSAVYLCASSSMATGGVVGDTQYFGPGTRLTVLEDLRNEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

(19) United States
(12) Patent Application Publication
(10) Pub. No.: US 2025/0216381 A1
(43) Pub. Date: Jul. 3, 2025

(54) Methods for Identifying Neoantigen-Reactive T Cell Receptors

(71) Applicant: Alaunos Therapeutics, Inc., Houston, TX (US)

(72) Inventors: Matthew R. COLLINSON-PAUTZ, Houston, TX (US); Drew C. DENIGER, Houston, TX (US); Guowei GU, Houston, TX (US); Ana KORNGOLD, Houston, TX (US); Beatriz A. SANTILLAN, Houston, TX (US); Hao ZHAO, Houston, TX (US)

(73) Assignee: Alaunos Therapeutics, Inc., Houston, TX (US)

(21) Appl. No.: 18/847,945

(22) PCT Filed: Mar. 21, 2023
(86) PCT No.: PCT/US2023/015832

§ 371 Date: Sep. 17, 2024

Related U.S. Application Data
(60) Provisional application No. 63/322,220, filed on Mar. 21, 2022.
(60) Provisional application No. 63/382,522, filed on Nov. 6, 2022.

(51) Int. Cl. G01N 33/50; C07K 14/725; C07K 14/74; C12N 5/0783; C12N 15/85; C12Q 1/6881

(52) U.S. Cl. CPC G01N 33/505; C07K 14/7051; C07K 14/70539; C12N 5/0636; C12N 15/85; C12Q 1/6881; C12Q 2600/156; C12Q 2600/158; G01N 2333/7051

(57) ABSTRACT

The present disclosure provides methods for identifying novel neoantigen-reactive T cell receptors (TCRs) by co-culturing a reporter T cell comprising a TCR expression cassette and an antigen presenting cell expressing a target neoantigen sequence and a matched human lymphocyte antigen (HLA) sequence. The present disclosure also provides novel neoantigen-reactive TCRs and the use thereof.

### CROSS-REFERENCE TO RELATED APPLICATIONS

This application claims the benefit of U.S. Provisional Application No. 63/322,220, filed Mar. 21, 2022, and U.S. Provisional Application No. 63/382,522, filed Nov. 6, 2022, both of which are incorporated by reference in their entireties herein.

### FIELD

The present disclosure relates to the identification of T cell receptors with defined antigen and HLA specificity and methods of using the same.

### BACKGROUND

Rapid and accurate identification of T cell receptors (TCRs) with defined antigen and HLA specificity has the potential to enable the discovery of TCRs with therapeutic applications. Individual T cell receptors (TCRs) can generally be defined with three key pieces of information; 1) Full-length paired (e.g., α and β) TCR sequence, 2) Antigenic specificity and 3) HLA-restriction. Obtaining this information from a highly polyclonal population of T cells, such as those from peripheral blood or within tissues (e.g., tumor specimens) is challenging to do in an accurate and efficient manner.

At the intersection of cutting-edge technologies and robust immunological assay systems, a platform for overcoming this challenge has been developed and is provided in the present disclosure.

### SUMMARY

The present disclosure provides a method for identifying a neoantigen-reactive TCR, comprising: i) co-culturing a) a reporter T cell comprising a TCR expression cassette, and b) an antigen presenting cell (APC) that expresses a target neoantigen sequence and a matched human leukocyte antigen (HLA) sequence; and ii) identifying a positive reporter signal in the reporter T cell to identify a neoantigen-reactive TCR. In one aspect, the methods disclosed herein comprises identifying TCR sequences from tumor infiltrating lymphocytes (TILs) isolated from a tumor sample. In another aspect, the methods further comprise identifying somatic mutations in the tumor sample and determining the germline HLA typing of the tumor sample.

The present disclosure provides a method of identifying a neoantigen-reactive T cell receptor (TCR), comprising: i) obtaining TCR α and β chain sequences from TILs isolated from a tumor sample; ii) obtaining neoantigen sequences comprising somatic mutations present in the tumor sample, and the germline HLA typing of the tumor sample; iii) co-culturing a) a reporter T cell expressing a TCR sequence reconstructed from the TCR α and β chain sequences obtained in step i), and b) an antigen presenting cell (APC) that expresses a neoantigen sequence and a matched human leukocyte antigen (HLA) sequence obtained in step ii); and iv) evaluating the reporter activity in the reporter T cell to identify a neoantigen-reactive TCR.

In one aspect, the present disclosure provides a method for identifying a neoantigen-reactive TCR, comprising: i) obtaining single-cell gene expression profiles from a population of tumor infiltrating lymphocytes (TIL) isolated from a patient sample, ii) performing bioinformatics analyses on the single cell gene expression data to identify TCR clonotypes, clustering the TCR clonotypes and to select a clonotype of interest, iii) creating recombinant alpha and beta TCR sequences in silico and preparing a reporter T cell comprising a TCR expression cassette encoding a TCR sequence reconstructed from paired TCR α and β chain sequences identified from the clonotype of interest in step ii), iv) preparing a tandem minigene (TMG) expression vector comprising nucleic acid sequences for the expression of concatenated amino acid sequences of non-synonymous single nucleotide variants (SNVs); v) analyzing the patient sequencing data to identify class I and class II HLA alleles and preparing HLA expression vectors comprising the class I HLA and class II HLA allele sequences; vi) preparing an APC comprising transfecting said TMG expression vector and one or more HLA expression vectors into a cell wherein each transfection condition comprises a TMG and one or two HLA types; vii) co-culturing the reporter T cell in step iii) with the APC of step vi), and viii) identifying a positive reporter activity in the reporter T cell to identify a neoantigen-reactive TCR. In certain aspect, the clustering comprises grouping the TCR clonotypes by CD8 or CD4 expression, gene function of differentially expressed genes, and the level of expression of each TCR. In some aspects, the method comprises preparing an APC comprising transfecting the TMG expression vector and up to four, up to five, up to six, up to seven, up to eight, up to nine, up to ten, up to eleven, up to twelve, up to thirteen, up to fourteen, up to fifteen, up to sixteen, up to seventeen, up to eighteen, up to nineteen, or up to twenty HLA expression vectors into a cell. In one aspect, up to eight HLA expression vectors are transfected into a cell in the TCR screening protocol disclosed herein. In another aspect, up to seventeen HLA expression vectors are transfected into a cell in the TIL screening protocol disclosed herein. In some aspects, the method comprises pulsing neoantigen peptides into a cell instead of transfecting the cell with a TMG expression vector.

In a further aspect, the present disclosure provides a method for identifying a neoantigen-reactive TCR, comprising: i) obtaining single-cell gene expression profiles from a population of tumor infiltrating lymphocytes (TIL) isolated from a patient sample and whole exome sequence (WES) data from the patient sample, ii) performing bioinformatics analysis on the single cell gene expression data to identify TCR clonotypes of interest, iii) creating recombinant TCR sequences, iv) preparing a reporter T cell comprising a TCR expression cassette encoding a TCR sequence reconstructed from paired TCR α and β chain sequences identified from the clonotype of interest in step ii), v) preparing a tandem minigene (TMG) expression vector; vi) identifying class I and class II HLA alleles and preparing HLA expression vectors comprising the class I HLA and class II HLA allele sequences; vii) preparing an APC comprising transfecting said TMG expression vector and up to four HLA expression vectors into a cell wherein each transfection condition comprises a TMG and one or two HLA types; viii) co-culturing the reporter T cell in step iii) with the APC of step vi), and ix) identifying a positive reporter activity in the reporter T cell to identify a neoantigen-reactive TCR. In certain aspect, the clustering comprises grouping the TCR clonotypes by CD8 or CD4 expression, gene function of differentially expressed genes, and the level of expression of each TCR.

The present disclosure also provides a co-culture reporter system for identifying a T cell receptor (TCR) that recognizes a target neoantigen, comprising: i) a reporter T cell comprising a TCR expression cassette, co-cultured with ii) an antigen presenting cell (APC) that expresses a target neoantigen sequence and a matched human leukocyte antigen (HLA) sequence.

In one aspect, the TCR expression cassette as disclosed herein comprises a TCR sequence reconstructed from TCR α and β chain sequences identified from TILs isolated from a tumor sample, and wherein the target neoantigen sequence and the matched HLA sequence are identified from the same tumor sample. Methods of identifying TCR sequences, antigen or neoantigen sequences, or the HLA sequences from a tumor sample or a normal reference sample are known in the art. Some of the commonly used methods are also described herein.

In one aspect, the isolated TILs are first expanded ex vivo and then co-cultured with APCs modified to express relevant HLA alleles and antigens obtained from the tumor sample. In a further aspect, a gene signature for identifying neoantigen reactive TCRs from ex vivo expanded TILs includes one or more gene(s) selected from the group consisting of XCL2, XCL1, IL2, CSF2, IFNG, CCL4, CCL4L2, TNF, CCL3, RGCC, TNFSF9, DUSP2, NFKBID, MIR155HG, NR4A3, EVI2A, CRTAM, ZBED2, FABP5, PIM3, NR4A1, IL10, TNFSF14, NR4A2, LINC00892, ZFP36L1, GZMB, MYC, SPRY1, KDM6B, EGR2, PHLDA1, PPPIR2, VSIR, REL, PRDX1, SLA, CYTOR, DDX21, IER3, PGAM1, NAMPT, HSP90AB1, IL23A, FAM107B, BCL2A1, ZEB2, ZBTB32, BTG2, GADD45B, RILPL2, SEMA7A, TGIF1, SRGN, RAN, CFLAR, MAT2A, SIAH2, PRNP, RNF19A, FASLG, NME1, EVI2B, HSPH1, NOP16, CSRNP1, and TAGAP.

In one aspect, the reporter T cell disclosed herein is a primary T cell. In another aspect, the reporter T cell disclosed herein is from an immortalized T cell line. In a certain aspect, the reporter T cell disclosed herein is not a primary T cell. In certain aspects, the immortalized cell is a Jurkat cell or a SUP-T1 cell. In some aspects, the Jurkat cell is Jurkat NFAT. In one aspect, the endogenous T cell receptor of the cells is downregulated or knocked out, such as using routine methods in the art.

In one aspect, the reporter T cell disclosed herein expresses any or all protein components of the TCR signaling complex or downstream signaling components. In a certain aspect, the reporter T cell expresses one or more components selected from the group consisting of CD3, CD4, CD8a, and CD8b. In further aspects, these protein components are modified, such as by mutation of one or more amino acids, to enhance their activities.

In one aspect, the antigen presenting cell (APC) disclosed herein is a classical professional APC. In another aspect, the APCs disclosed herein are artificial APCs. In a certain aspect, the APC disclosed herein is not a professional APC. In certain aspects, the APC used in the methods or cell systems disclosed herein is a COS cell. In one aspect, the COS cell is a COS-7 cell. In one aspect, the APC is a 293-HEK cell. In another aspect, the APC is not a 293-HEK cell. In one aspect, the APC endogenously expresses an HLA allele. In another aspect, the APC does not express any endogenous HLA. In one aspect, the APC comprises one or more HLA expression plasmids. In one aspect, the APC expresses multiple HLA alleles in a single cell.

In one aspect, the APC expresses a co-stimulatory molecule. Examples of the co-stimulatory molecules include, but not limited to, 4-1BBL, CD40, CD80, CD86, or OX40L.

In some aspects, the reporter T cell disclosed herein comprises a reporter system that is activated by the binding of a TCR to an antigen. Examples of the reporter systems are known in the art and include, but are not limited to, systems based on luciferase activity, fluorescence, or cytokine production.

In one aspect of the present disclosure, the reporter T cells and the APCs are co-cultured at a ratio from about 16:1 to about 1:16. In one aspect, the reporter T cells and the APCs are co-cultured at a ratio of about 4:1. In another aspect, the reporter T cells and the APCs are co-cultured at a ratio of about 8:1. In certain aspect, the reporter T cells and the APCs are co-cultured at a ratio of about 1:16, 1:8, 1:4, 1:2, 1:1, 2:1, 4:1, 8:1, or 16:1.

In one aspect, the reporter T cells and the APCs are co-cultured for 1-48 hours. In another aspect, the reporter T cells and the APCs are co-cultured for about one hour, about 2 hours, about 3 hours, about hours, at least 5 hours, about 6 hours, about 7 hours, about 8 hours, about 9 hours, or about 10 hours.

The present disclosure provides TCR sequences, or an antigen-binding portion thereof, that are identified or obtained by any of the methods disclosed herein. In one aspect, a TCR sequence comprises one or more of the sequences selected from the group consisting of SEQ ID NOs: 1-300, 536-1003, and 1025-1204 (the sequences provided in Tables 1-79). In another aspect, a TCR sequence comprises a sequence that is at least 80%, at least 85%, at least 90%, or at least 95% identical to a sequence selected from the group consisting of SEQ ID NOs: 1-300, 536-1003, and 1025-1204 (the sequences provided in Tables 1-79).

The present disclosure provides a polynucleotide encoding an amino acid sequence that is at least 90%, 91%, 92%, 93%, 94%, 95%, 96%, 97%, 98%, 99%, or 100% identical to an amino acid sequence selected from the group consisting of SEQ ID NOs: 1-300, 536-1003, and 1025-1204 (the sequences provided in Tables 1-79).

The present disclosure also provides a neoantigen/HLA complex, where the neoantigen comprises a sequence selected from the group consisting of SEQ ID NOs: 310 to 535 and wherein the HLA comprises a sequence selected from a group consisting of SEQ ID NOs: 301 to 309.

The present disclosure also provides recombinant vectors expressing a TCR, or an antigen-binding portion thereof, that are disclosed herein. Production of recombinant vectors is well-known in the art, and a variety of vectors may be utilized, including viral or non-viral vectors. In some aspects, the recombinant vector comprises a polycistronic expression cassette, wherein the polycistronic expression cassette comprises a transcriptional regulatory element operably linked to a polycistronic polynucleotide that comprises: a) a first polynucleotide sequence that encodes a T cell receptor (TCR) alpha chain comprising an alpha chain variable (Vα) region and an alpha chain constant (Cα) region; b) a second polynucleotide sequence that comprises a first 2A element; c) a third polynucleotide sequence that encodes a TCR beta chain comprising a beta chain variable (Vβ) region and a beta chain constant (Cβ) region; d) a fourth polynucleotide sequence that comprises a second 2A element; and e) a fifth polynucleotide sequence that encodes a fusion protein that comprises IL-15, or a functional fragment or functional variant thereof, and IL-15Rα, or a functional fragment or functional variant thereof. In one aspect, the recombinant vector o comprises the first, the second, the third, the fourth, and the fifth polynucleotide sequence in any order from 5′ to 3′. In some aspects, the TCR alpha chain comprises an amino acid sequence at least 90%, 91%, 92%, 93%, 94%, 95%, 96%, 97%, 98%, 99%, or 100% identical to the amino acid sequence of TCR alpha chain sequences disclosed in Tables 1-79, and the TCR beta chain comprises an amino acid sequence at least 90%, 91%, 92%, 93%, 94%, 95%, 96%, 97%, 98%, 99%, or 100% identical to the amino acid sequence of TCR beta chain sequences disclosed in Tables 1-79.

The present disclosure further provides a population of cells that comprise the recombinant vectors disclosed herein. In one aspect, the recombinant vector or the polynucleotide is integrated into the genome of the population of cells. In one aspect, the cells are immune effector cells. In certain aspects, the immune effector cells are selected from the group consisting of T cells, natural killer (NK) cells, B cells, mast cells, and myeloid-derived phagocytes.

The present disclosure provides a pharmaceutical composition comprising a population of cells as disclosed herein. In one aspect, the pharmaceutical composition comprises a pharmaceutically acceptable carrier.

The present disclosure further provides a method to treat or to prevent a medical condition, comprising administering a pharmaceutical composition described herein to a patient in need. In one aspect, the medical condition is a cancer.

### BRIEF DESCRIPTION OF THE DRAWINGS

FIG. 1 is a schematic of the TCR identification and screening platform.

FIG. 2 is a schematic showing the process for screening of TCRs obtained from TILs.

FIG. 3 is a schematic showing the comparison of TCR-based screening and TILs-based screening methods.

FIG. 4 presents the results of lentivirus infection of Jurkat NFAT cells to form CD8Lenti cells subsequently infected with CD4 lentivirus, as described in the Examples. Cells are harvested and stained with CD3, CD4, CD8A and CD8B. Jurkat NFAT parental cells are negative for CD8 (99.16% CD8 negative) on CD3+ cells. The results in FIG. 2 show that Jurkat LentiCD8 cells line have 43.57% CD8a expression and 43.56% CD8a and CD8b double positive expression.

FIG. 5 presents the results of single clones with high CD8 and luciferase activity signal to noise ratio. PBMCs from 3 different donors are irradiated and seeded in 96 multiwell plates at 100 k/well. Puromycin-selected Jurkat NFAT CD8Lenti stable pool cells are seeded at 0.5 cell/well on top of irradiated PBMCs (96 multiwells) to generate single clones. Single clones are cultured for 1 week with IL-2 (50 IU/ml) and PHA (0.25 μg/ml). Second week cell medium is replaced with 100 IU/ml of IL-2. Grown back clones are evaluated for CD8a and CD8b expression and luciferase signal/noise ratio (PMA/Ionomycin vs untreated). FIG. 5 shows that clones #2, 15, 19, 41 (>95% CD8 expression and >150 signal to noise ratio) are the best clones with higher CD8 expression and higher luciferase activity signal to noise ratio.

FIG. 6 presents the results of flow cytometry analysis showing that cells from the Jurkat NFAT_CD8Lenti pool have 46.74% CD8A and CD8B double positive cells. Cells from the #41 clone show 95.74% CD8A and CD8B double positive cells.

FIG. 7 presents the results of flow cytometry analysis showing that cells from the Jurkat NFAT_CD8Lenti clone #41 infected with pGenLenti-CD4_IRES_Puro lentivirus have a CD4 positive population of 97.8%, compared to cells not infected.

FIG. 8 presents the results of single clones with high CD8 and luciferase activity signal to noise ratio. Jurkat cells are seeded in RPMI complete medium at 200 k cells/well in 96 multiwells. Cells are treated with PMA 50 ng/ml and Ionomycin 1 μg/ml for 2.5H, 3.5H, 4.5H and 5.5H. Cells are harvested and lysed with passive lysis buffer (Promega) at room temperature for 15 minutes. 50 μL of cell lysis are mixed with 100 μl of luciferase substrate (Promega). Luciferase signal intensities are detected with Luminometer. Luciferase activity folds changes are calculated by dividing PMA/Ionomycin treated condition to vehicle control treated conditions. FIG. 8 provides that 4-5 hours is the best time period to harvest cells since signals start to drop from CD8Lenti_CD4Lenti pool.

FIG. 9A presents the results of co-culturing Jurkat NFAT CD8Lenti cells with COS-7 cells transfected with TMG1 or TMG2 with 75 ng of HLA A*11:01 and 75 ng of HLA A*02:01. FIG. 9B presents TCR-mediated reporter activity in Jurkat NFAT cells expressing CD8 co-receptor.

FIG. 10 presents Jurkat NFAT cells with or without CD8 co-receptor electroporated with Curie, McClintock cells and stained with CD3, CD4, CD8a, CD8b and mTCR antibodies. Cells are analyzed using flow cytometry to detect the percentage of cells with mTCR expression. Cells are stained with CD3, CD4, CD8a, CD8b and mTCR antibodies. As shown, cells express similar level of mTCR in Jurkat NFAT CD8Lenti cells compared with Jurkat NFAT parental cells. Over 90% of cells are viable in all six cell lines on the next day after electroporation suggesting NEON electroporation system provides highly viable T cells with sufficient percentage of mTCR expression (˜20%). This allows coculture experiments to be performed next day without wasting time to recover cells.

FIG. 11 presents flow cytometry analysis results for mTCR expression level in 11 TCRs for cells stained with CD3, CD4, CD8a, CD8b antibodies and mTCR antibodies. FIG. 11 shows that mTCR expression varied from 8-35% (9 of 11 TCRs expressed above 15%) when cells are gated on CD3+.

FIG. 12 presents the results of a luciferase activity, indicating the specificity of TMGs matched to 9 of 11 TCRs.

FIG. 13 presents the results of an experiment designed to troubleshoot samples with low TCR reactivity of FIG. 12. COS-7 cells are transfected with 75 ng of plasmids compared to the COS-7 cells transfected with 25 ng of plasmids in FIG. 9.

FIG. 14 presents results for peptide pulsing with long and short peptides and library TCRs.

FIG. 15 presents the results of the development of an anti-TCR positive control using Jurkat cells electroporated with various TCRs and H57 anti-TCR antibody coated multiwell plate.

FIG. 16 presents a scatter plot showing luciferase activity from anti-TCR positive control (FIG. 15, “Anti-TCR (Pos. Ctrl)”) vs. the percent expression of the electroporated TCR as measured by flow cytometry. A trend line (linear regression) is shown as a dotted line. The linear regression model and R2 values are shown on the plot.

FIG. 17A-B show luciferase activity fold change of Jurkat cells electroporated with 18 different TCRs in response to TMG1 or TMG2 and HLA-A & HLA-B (filled circle), HLA-C (square), HLA-DQ (filled triangle), and HLA-DP & HLA-DR (circle).

FIG. 18 presents luciferase activity of electroporated Jurkat cells plated, for 5 hours, onto a 96 multiwell plate coated with H57 antibody. All cells with electroporated TCRs show higher luciferase activity in H57 coated condition showing that TCRs are biologically functional.

FIG. 19 presents HLA allele specificity by analyzing luciferase activity of COS-7 cells transfected with individual HLA and TMG1. HLA-A* 03:01 is the specific HLA that TCR12 is reactive to.

FIG. 20 shows reversion TMG for TMG1 designed to determine which mutation in the TMG1 is specifically being recognized by TCR12. FIG. 20 shows that ERGIC2 L176P is the one with lower luciferase activity after co-culture, suggesting that this mutation plays a critical role for TCR12 reactivity. Peptide prediction online tools are used to predict some potential candidates with minimal residue number of peptide likely to bind with HLA *03:01. The long peptide of 25mer did not work for TCR12 specificity test since some Class I TCRs could not work with the long peptide.

FIG. 21 shows a peptide prediction online tool to predict some potential candidates with minimal residue of peptide likely to bind with HLA-A *03:01. ERGIC2 L176P 10mer was specific to TCR12 which confirmed with TMG reversion data.

FIG. 22 is an example of a plate layout to screen a TCR for patient 8434.

FIG. 23 presents HLA clusters transfected into COS-7 cells for screening patient 8434 TCRs.

FIG. 24A shows a TCR reactive to the same combination of HLA group B and TMG2 in patient 8434. This TCR is clonotype 3. HLA cluster B (which had HLA B*35:02, C*06:02 and C*04:01).

FIG. 24B shows a TCR reactive to the same combination of HLA group E and TMG1 in patient 8434. This TCR is clonotype 20. HLA cluster E had DRA*01:01, DRB1*11:01 and DRB3*02:02.

FIG. 24C shows a TCR reactive to the same combination of HLA group E and TMG1 in patient 8434. This TCR is clonotype 21. HLA cluster E had DRA*01:01, DRB1*11:01 and DRB3*02:02.

FIG. 24D shows a TCR reactive to the same combination of HLA group E and TMG1 in patient 8434. This TCR is clonotype 23. HLA cluster E had DRA*01:01, DRB1*11:01 and DRB3*02:02.

FIG. 24E shows a TCR reactive to the same combination of HLA group B and TMG2 in patient 8434. This TCR is clonotype 27. HLA cluster B (which had HLA B*35:02, C*06:02 and C*04:01).

FIG. 25A presents results of HLA parsing of patient 8434 reactive TCRs. HLA-A*35:02 is the specific HLA that TCR3 is reactive to.

FIG. 25B presents results of HLA parsing of patient 8434 reactive TCRs. HLA DRB1*11:01 is the specific HLA that TCR20 is reactive to.

FIG. 25C presents the results of HLA parsing of patient 8434 reactive TCR21. HLA DRB1*11:01 was the specific HLA that TCR21 is reactive to.

FIG. 25D presents the results of HLA parsing of patient 8434 reactive TCR23. HLA DRB1*11:01 is the specific HLA that TCR23 is reactive to.

FIG. 25E presents the results of HLA parsing of patient 8434 reactive TCR27. HLA-A*35:02 was the specific HLA that TCR27 is reactive to.

FIG. 26A presents the results of an experiment to identify which neoantigen is recognized by the TCR. 12 peptides encoded within TMG2 are pulsed separately and demonstrated that number 8 peptide on this TMG is the shared peptide for 8434-TCR3 and 8434-TCR27. The mutation is KRAS p.Q61H with allele frequency 0.423 in the WES data suggesting that it is a clonal mutation in the patient tumor.

FIG. 26B presents the results of an experiment to determine which neoantigen is involved in the TCR-neoantigen reactivity. 12 peptides are pulsed in the TMG1 separately. Number 9 peptide on this TMG is the shared peptide for 8434-TCR20, 8434-TCR21, and 8434-TCR23. This mutation is ARHGEF16 p.R150W with allele frequency 0.193 in the WES data suggesting that it is a sub-clonal mutation in this patient tumor.

FIG. 26C presents the results of an experiment to determine which neoantigen is involved in the TCR-neoantigen reactivity. 12 peptides are pulsed in the TMG1 separately and demonstrated that number 9 peptide on this TMG is the shared peptide for these 3 TCRs. This mutation is ARHGEF16 p.R150W with allele frequency 0.193 in the WES data suggesting that it is a sub-clonal mutation in this patient tumor.

FIG. 26D presents results of an experiment to determine which neoantigen is involved in the TCR-neoantigen reactivity. 12 peptides are pulsed in the TMG1 separately and demonstrated that number 9 peptide on this TMG is the shared peptide for these 3 TCRs. This mutation is ARHGEF16 p.R150W with allele frequency 0.193 in the WES data suggesting that it is a sub-clonal mutation in this patient tumor.

FIG. 26E presents results of an experiment to determine which neoantigen was involved in the TCR-neoantigen reactivity. 12 peptides are pulsed in the TMG2 separately and demonstrated that number 8 peptide on this TMG is the shared peptide for these 2 TCRs. This mutation is KRAS p.Q61H with allele frequency 0.423 in the WES data suggesting that it is a clonal mutation in this patient tumor.

FIG. 27 presents results of IFN-γ ELISpot Spot forming colonies in patient 8434 TILs co-cultured with APCs. TMG2 and top-spot TMG9 (which contains KRAS p.Q61H same as TMG2) had higher signal compared with other TMGs. HLA group 2 which included HLA B*35:02 and HLA B*47:01 had strongest signal in both TMG2 and top-spot TMG9. HLA expression of COS-7 cells are measured with flow cytometry using antibodies cocktail HLA-A2, HLA-DP, HLA-DQ and HLA-DR.

FIG. 28 presents the results of 4-1BB expression of patient 8434 TILs co-cultured with APCs. TMG2 and top-spot TMG9 (which contains KRAS p.Q61H same as TMG2) had higher signal compared with other TMGs. HLA group 2 which included HLA B*35:02 and HLA B*47:01 had strongest signal in both TMG2 and top-spot TMG9. HLA expression of COS-7 cells are measured with flow cytometry using antibodies cocktail HLA-A2, HLA-DP, HLA-DQ and HLA-DR.

FIG. 29 presents an evaluation of 4-1BB expression on T cells in co-cultures to reveal that addition of CD80, CD86, and OX40L, but not 4-1BBL or CD40 increased the measured 4-1BB upregulation in activating conditions (i.e., HLA Group 2+TopSpot TMG9) while having little to no effect in non-activating conditions (i.e., HLA Groups 1 or 2+TopSpot TMG9 or HLA Groups 1-3+Irrelevant TMG).

FIG. 30 presents the results of FAC-sorting of patient 8434 TILs after co-culture with APCs. Patient 8434 TILs are cocultured with COS-7 cells transfected with TMG2 and HLA B based on the ELISpot data analysis (STIM). COS-7 parental cells are incubated with TILs as negative control (NTC). We have incubated COS-7 cells and TILs for 4 hours and overnight. Cells are sorted from SONY SH800 using viability dye, CD3, CD4, CD8 and 41BB antibodies. Cells are sorted on lymphocyte and live cells as NEAT for both 4 hours and overnight. Enough cells are recovered to run 10× to target 10,000 cells. Viability is 99% for 4 hours both NTC and STIM conditions. Viability is 93% and 100% for overnight NTC or STIM conditions respectively. At 4 hours, 41BB is expressed at 4.07% in the STIM sample compared with 0.37% in the NTC samples on the CD3+CD8+ gate. In addition, 41BB is expressed at 8.52% in STIM sample compared with 0.01% in the NTC sample after overnight. This suggested that there are a substantial number of cells being activated after culture with COS-7 cells in STIM condition.

FIG. 31 presents cluster analysis of TILs after the 4 hr co-culture provided in FIG. 30.

FIG. 32 presents cluster analysis of TILs after the overnight co-culture provided in FIG. 30.

FIG. 33 shows the HLA clusters used for transfection of the APCs in the TCR screening co-culture assay to test TCRs from patient 6932.

FIG. 34 shows a heatmap of reporter activity in TCR-modified reporter cells for the reactive TCR (6932-TCR5) from patient 6932. Each condition is tested in duplicate and the reporter activity for each replicate is shown in the wells.

FIG. 35 shows a heatmap of reporter activity TCR-modified reporter cells from TCR 6932-TCR5 from patient 6932. TCR-modified reporter cells are co-cultured with APCs modified with the indicated HLA alleles and pulsed with the neoantigen peptides indicated along the vertical axis.

FIG. 36 shows the HLA clusters used for transfection of the APCs in the TCR screening co-culture assay to test TCRs from patient 0025.

FIG. 37A-R shows a heatmap of reporter activity in TCR-modified reporter cells for reactive TCRs from patient 0025. Each of these TCRs is reactive towards at least one combination of HLA and TMG evaluated. Each condition is tested in duplicate and the reporter activity for each replicate is shown in the wells.

FIG. 38 is an example of a plate layout to screen a TCR from Patient 9976.

FIG. 39 shows representative results of HLA specificity when screening TCRs from Patient 9976.

FIG. 40 presents the results for the mutation (panel A) and HLA allele (panel B) specificity for TCR38-2 from Patient 9976.

FIG. 41 presents the results of TCR38-2 reactivity against different KRAS mutations.

FIG. 42 shows representative results of HLA specificity when screening TCR10-TCR16 from Patient 7014.

FIG. 43 shows representative results of HLA specificity when screening TCR44-TCR51 from Patient 7014.

FIG. 44 shows representative results of HLA specificity when screening TCR52-TCR55 from Patient 7014.

FIG. 45 presents the results for the mutation (panel A) and HLA allele (panel B) specificity for TCR16 from Patient 7014.

FIG. 46 presents the results for the mutation (panel A) and HLA allele (panel B) specificity for TCR51 from Patient 7014.

FIG. 47 presents the results for the mutation (panel A) and HLA allele (panel B) specificity for TCR55 from Patient 7014.

FIG. 48 presents the specificity of TCR3 (panel A) or TCR27 (panel B) for KRAS mutation, as measured by up-regulation of interferon gamma.

FIG. 49 presents the specificity of TCR3 (panel A) or TCR27 (panel B) for KRAS mutation, as measured by up-regulation of 4-1BB.

FIG. 50 presents the results of tumor killing by neoantigen-reactive TCR3 and TCR27.

FIG. 51A-51E shows effector T cells phenotype of TCR-T cells cultured with IL-15 complex and restimulated TCR-T cells expressing mbIL-15 and after reactivation from long-term cytokine withdrawal (LTWD). The data is presented as (A) pseudocolor plots showing the expression of CD45RA and CD45RO (upper plots) and CD95 and CD62L (lower plots), (B) pie charts showing the frequency of the different subsets identified by Boolean gating; (C) pseudocolor plots showing mTCR and mbIL-15 expression in CD3+ T cells; (D) histograms showing CellTrace Violet dilution in CD3+ T cells; and (E) a bar graph showing the percentage of CD3+ T cells survival when treated with or without mbIL-15. Representative of 2 donors.

### DETAILED DESCRIPTION

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

As used herein, the terms “about” and “approximately,” when used to modify a numeric value or numeric range, indicate that deviations of 5% to 10% above (e.g., up to 5% to 10% above) and 5% to 10% below (e.g., up to 5% to 10% below) the value or range remain within the intended meaning of the recited value or range.

As used herein, the terms “T cell receptor” and “TCR” are used interchangeably and refer to molecules comprising CDRs or variable regions from α3 T cell receptors. Examples of TCRs include, but are not limited to, full-length TCRs, antigen-binding fragments of TCRs, soluble TCRs lacking transmembrane and cytoplasmic regions, single-chain TCRs containing variable regions of TCRs attached by a flexible linker, TCR chains linked by an engineered disulfide bond, single TCR variable domains, single peptide-HLA-specific TCRs, multi-specific TCRs (including bispecific TCRs), TCR fusions, TCRs comprising co-stimulatory regions, human TCRs, humanized TCRs, chimeric TCRs, recombinantly produced TCRs, and synthetic TCRs. In certain embodiments, the TCR is a full-length TCR comprising a full-length α chain and a full-length β chain. In certain embodiments, the TCR is a soluble TCR lacking transmembrane and/or cytoplasmic region(s). In certain embodiments, the TCR is a single-chain TCR (scTCR) comprising Vα and Vβ linked by a peptide linker, such as a scTCR having a structure as described in PCT Publication No.: WO 2003/020763, WO 2004/033685, or WO 2011/044186, each of which is incorporated by reference herein in its entirety. In certain embodiments, the TCR comprises a transmembrane region. In certain embodiments, the TCR comprises a co-stimulatory signaling region.

As used herein, the term “full-length TCR” refers to a TCR comprising a dimer of a first and a second polypeptide chain, each of which comprises a TCR variable region and a TCR constant region comprising a TCR transmembrane region and a TCR cytoplasmic region. In certain embodiments, the full-length TCR comprises one or two unmodified TCR chains, e.g., unmodified a or 3TCR chains. In certain embodiments, the full-length TCR comprises one or two altered TCR chains, such as chimeric TCR chains and/or TCR chains comprising one or more amino acid substitutions, insertions, or deletions relative to an unmodified TCR chain. In certain embodiments, the full-length TCR comprises a mature, full-length TCR α chain and a mature, full-length TCR β chain.

The “antigen-binding portion” of the TCR, as used herein, refers to any portion comprising contiguous amino acids of the TCR of which it is a part, provided that the antigen-binding portion specifically binds to the target neoantigen as described herein with respect to other aspects of the disclosure. The term “antigen-binding portion” refers to any part or fragment of the TCR of the disclosure, which part or fragment retains the biological activity of the TCR of which it is a part (the parent TCR). Antigen-binding portions encompass, for example, those parts of a TCR that retain the ability to specifically bind to the target antigen, or detect, treat, or prevent a condition, to a similar extent, the same extent, or to a higher extent, as compared to the parent TCR.

As used herein, the term “TCR variable region” refers to the portion of a mature TCR polypeptide chain (e.g., a TCR α chain or β chain) which is not encoded by the TRAC gene for TCR α chains, either the TRBC1 or TRBC2 genes for TCR β chains, or the TRDC gene for TCR δ chains. In some embodiments, the TCR variable region of a TCR α chain encompasses all amino acids of a mature TCR α chain polypeptide which are encoded by a TRAV and/or TRAJ gene, and the TCR variable region of a TCR β chain encompasses all amino acids of a mature TCR β chain polypeptide which are encoded by a TRBV, TRBD, and/or TRBJ gene (see, e.g., Lefranc and Lefranc, (2001) “T cell receptor FactsBook.” Academic Press, ISBN 0-12-441352-8, which is incorporated by reference herein in its entirety). TCR variable regions generally comprise framework regions (FR) 1, 2, 3, and 4 and complementarity determining regions (CDR) 1, 2, and 3.

As used herein, the terms “α chain variable region” and “Vα” are used interchangeably and refer to the variable region of a TCR α chain.

As used herein, the terms “β chain variable region” and “Vβ” are used interchangeably and refer to the variable region of a TCR β chain.

As used herein in the context of a TCR, the term “CDR” or “complementarity determining region” means the noncontiguous antigen combining sites found within the variable regions of a TCR chain (e.g., an α chain or a β chain). These regions have been described in Lefranc, (1999) The Immunologist 7:132-136; Lefranc et al., (1999) Nucleic Acids Res 27:209-212; Lefranc (2001) “T cell receptor FactsBook.” Academic Press, ISBN 0-12-441352-8; Lefranc et al., (2003) Dev Comp Immunol. 27 (1):55-77; and in Kabat et al., (1991) “Sequences of protein of immunological interest,” each of which is herein incorporated by reference in its entirety. In certain embodiments, CDRs are determined according to the IMGT numbering system described in Lefranc (1999) supra. In certain embodiments, CDRs are defined according to the Kabat numbering system described in Kabat supra. In certain embodiments, CDRs are defined empirically, e.g., based upon a structural analysis of the interaction of a TCR with a cognate antigen (e.g., a peptide or a peptide-HLA complex). In certain embodiments, the α chain and β chain CDRs of a TCR are defined according to different conventions (e.g., according to the Kabat or IMGT numbering systems, or empirically based upon structural analysis).

As used herein, the term “constant region” with respect to a TCR refers to the portion of a TCR that is encoded by the TRAC gene (for TCR α chains) or either the TRBC1 or TRBC2 gene (for TCR β chains), optionally lacking all or a portion of a transmembrane region and/or all or a portion of a cytoplasmic region. In certain embodiments, a TCR constant region lacks a transmembrane region and a cytoplasmic region. A TCR constant region does not include amino acids encoded by a TRAV, TRAJ, TRBV, TRBD, TRBJ, TRDV, TRDD, TRDJ, TRGV, or TRGJ gene (see, e.g., “T cell receptor Facts Book,” supra).

As used herein, the terms “major histocompatibility complex” and “MHC” are used interchangeably and refer to an MHC class I molecule and/or an MHC class II molecule.

As used herein, the term “MHC class I” refers to a dimer of an MHC class I α chain and a Beta-2 microglobulin chain and the term “MHC class II” refers to a dimer of an MHC class II α chain and an MHC class II β chain.

As used herein, the terms “human leukocyte antigen” and “HLA” are used interchangeably and can also refer to the proteins encoded by the MHC genes. HLA-A, HLA-B, HLA-C, HLA-E, HLA-F, and HLA-G refer to major and minor gene products of MHC class I genes. HLA-DP, HLA-DQ, and HLA-DR refer to gene products of MHC class I genes, which are expressed on antigen-presenting cells, B cells, and T cells.

As used herein, the term “peptide-HLA complex” refers to an HLA molecule (HLA class I, II or III) with a peptide bound in the art-recognized peptide binding pocket of the HLA.

In some embodiments, the HLA molecule is a membrane-bound protein expressed on the cell surface. In some embodiments, the HLA molecule is a soluble protein lacking transmembrane or cytoplasmic regions.

Neoantigens are a class of cancer antigens which arise from cancer-specific mutations in expressed protein. As used herein, the term “neoantigen” relates to a peptide or protein expressed by a cancer cell that includes one or more amino acid modifications compared to the corresponding wild-type (non-mutated) peptide or protein that is expressed by a normal (non-cancerous) cell. A neoantigen may be patient specific. A “cancer-specific mutation” is a somatic mutation that is present in the nucleic acid of a tumor or cancer cell but absent in the nucleic acid of a corresponding normal, i.e., non-tumorous or non-cancerous, cell.

As used herein, the terms “T cell” and “T lymphocyte” are used interchangeably. In one aspect, the T cell is a primary T cell. In another aspect, the T cell is an immortalized T cell line. T cells can be obtained from numerous sources in a patient, including but not limited to tumor, blood, bone marrow, lymph node, the thymus, or other tissues or fluids. The T cells can include any type of T cell and can be of any developmental stage, including but not limited to, CD4+/CD8+ double positive T cells, CD4+ helper T cells, e.g., Th1 and Th2 cells, CD8+ T cells (e.g., cytotoxic T cells), tumor infiltrating cells (e.g., TILs), peripheral blood T cells, memory T cells, naive T cells, and the like. The T cells may be CD8+ T cells, CD4+ T cells, or both CD4+ and CD8+ T cells.

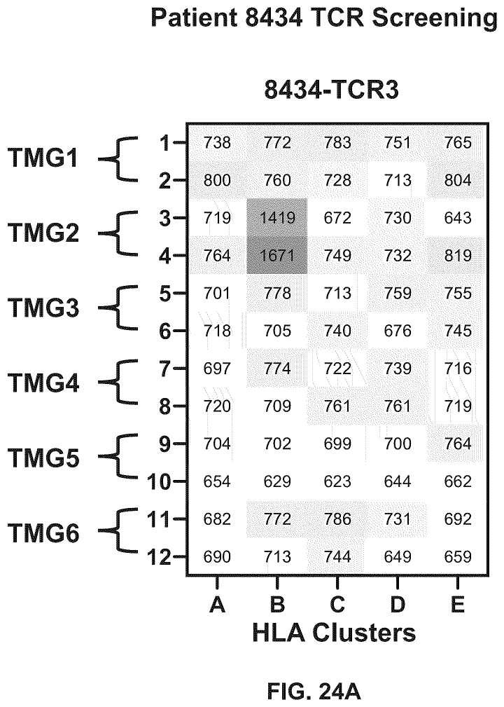

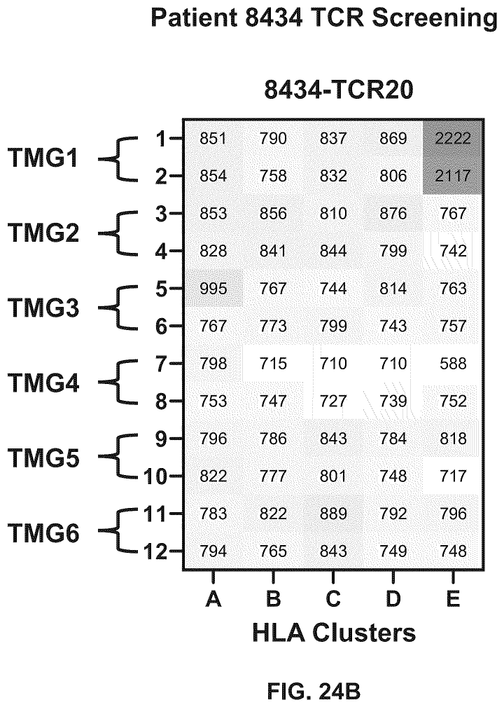

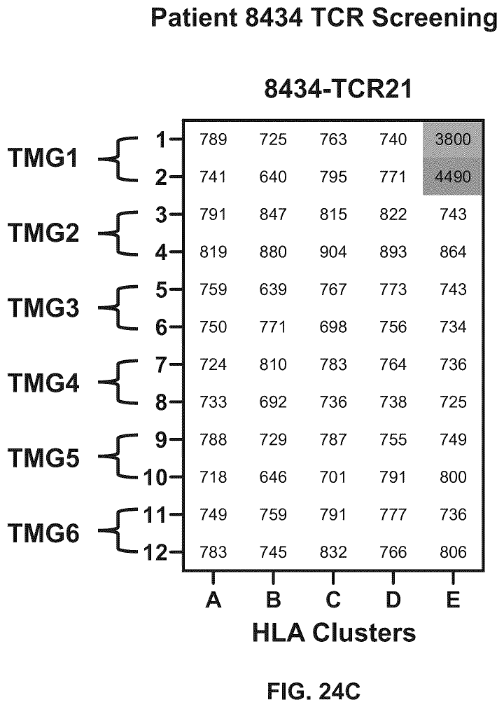

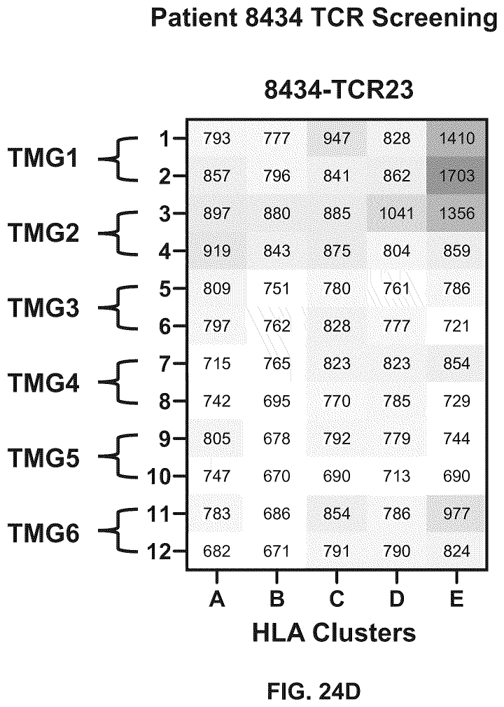

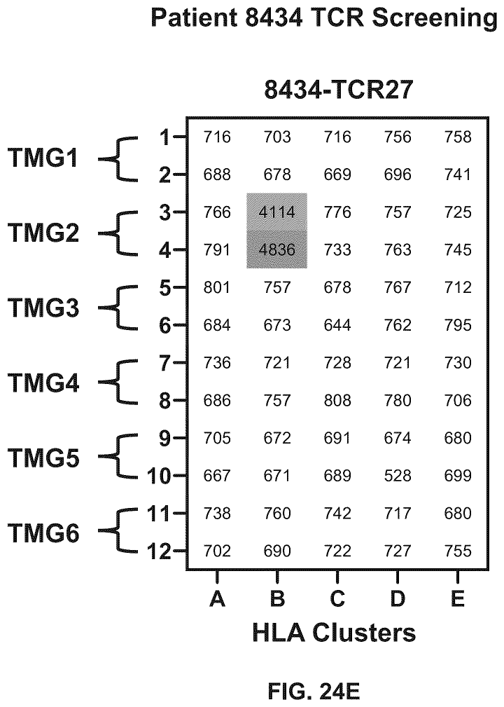

As used herein, the term “reporter T cell” refers to a T cell that comprises a TCR-mediated reporter system. Non-limiting examples of TCR-mediated reporter system include fluorescence-based systems, and those based on luciferase activity or cytokine production. See, e.g., Zong et al., 2020 PLOS ONE, and the references cited therein. A reporter system based on cytokine production may measure the production of one or more cytokines, the secretion of which by a T cell is characteristic of T cell activation (e.g., a TCR expressed by the T cells specifically binding to and immunologically recognizing the mutated amino acid sequence). Non-limiting examples of cytokines, the secretion of which is characteristic of T cell activation, include IFN-γ, IL-2, granzyme B, and tumor necrosis factor α (TNF-α), granulocyte/monocyte colony stimulating factor (GM-CSF), IL-4, IL-5, IL-9, IL-10, IL-17, and IL-22. In certain aspect, a “positive” reporter signal in a reporter T cell is a signal from a reporter gene that is at least 1.5× higher than the average of all of the samples when measured in a 96 well plate having a single TCR, up to 6 TMG sequences in duplicate and five different HLA clusters. In aspects, the reporter signal is luciferase activity. A positive reporter signal is detected when the TCR in the reporter T cell is paired with a matching APC comprising a TMG and matched HLA cluster. For example, as shown in FIG. 24.

The phrase “neoantigen-reactive,” as used herein, means that a TCR, or an antigen-binding portion thereof, can bind to and immunologically recognize the mutated amino acid sequence encoded by the cancer-specific mutation.

As used herein, the terms “treat,” “treating,” and “treatment” refer to therapeutic or preventative measures described herein. In some embodiments, the methods of “treatment” employ administration of a TCR or a cell expressing a TCR to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “subject” includes any human or non-human animal. In one embodiment, the subject is a human or non-human mammal. In one embodiment, the subject is a human.

As used herein, the term “polycistronic vector” refers to a polynucleotide vector that comprises a polycistronic expression cassette.

As used herein, the term “polycistronic expression cassette” refers to a polynucleotide sequence wherein the expression of three or more transgenes is regulated by common transcriptional regulatory elements (e.g., a common promoter) and can simultaneously express three or more separate proteins from the same mRNA. Exemplary polycistronic vectors, without limitation, include tricistronic vectors (containing three cistrons) and tetracistronic vectors (containing four cistrons).

As used herein, the term “polycistronic polynucleotide” refers to a polynucleotide that comprises three or more cistrons.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F, (1990) PNAS 87:2264-2268, modified as in Karlin S & Altschul S F, (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., at score=100, wordlength=12 to obtain nucleotide sequences homologous to a nucleic acid molecule described herein. BLAST protein searches can be performed with the XBLAST program parameters set, e.g., at score=50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules. Id. When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, (1988) CABIOS 4:11-17, which is herein incorporated by reference in its entirety. Such an algorithm is incorporated in the ALIGN program (version 2.0) which is part of the GCG sequence alignment software package.

The percent identity between two sequences can be determined using techniques similar to those described above, with or without allowing gaps. In calculating percent identity, typically only exact matches are counted.

The present disclosure provides a method for identifying a TCR that recognize a target neoantigen, comprising: i) co-culturing a) a reporter T cell comprising a TCR expression cassette, and b) an antigen presenting cell (APC) that expresses a target neoantigen sequence and a matched human leukocyte antigen (HLA) sequence; and ii) evaluating the reporter activity in the reporter T cell to identify a TCR that recognizes the target neoantigen. In one aspect, the methods disclosed herein comprises identifying TCR sequences from tumor infiltrating lymphocytes TILs isolated from a tumor sample. In another aspect, the methods further comprise identifying somatic mutations in the tumor sample and determining the germline HLA typing of the tumor sample.

The present disclosure provides a method of identifying a neoantigen-reactive T cell receptor (TCR), comprising: i) obtaining TCR α and β chain sequences from TILs isolated from a tumor sample; ii) obtaining neoantigen sequences comprising somatic mutations present in the tumor sample, and the germline HLA typing of the tumor sample; iii) co-culturing a) a reporter T cell expressing a TCR sequence reconstructed from the TCR α and β chain sequences obtained in step i), and b) an antigen presenting cell (APC) that expresses a neoantigen sequence and a matched human leukocyte antigen (HLA) sequence obtained in step ii); and iv) evaluating the reporter activity in the reporter T cell to identify a neoantigen-reactive TCR.

The present disclosure also provides a co-culture reporter system for identifying a T cell receptor (TCR) that recognizes a target neoantigen, comprising: i) a reporter T cell comprising a TCR expression cassette, co-cultured with ii) an antigen presenting cell (APC) that expresses a target neoantigen sequence and a matched human leukocyte antigen (HLA) sequence.

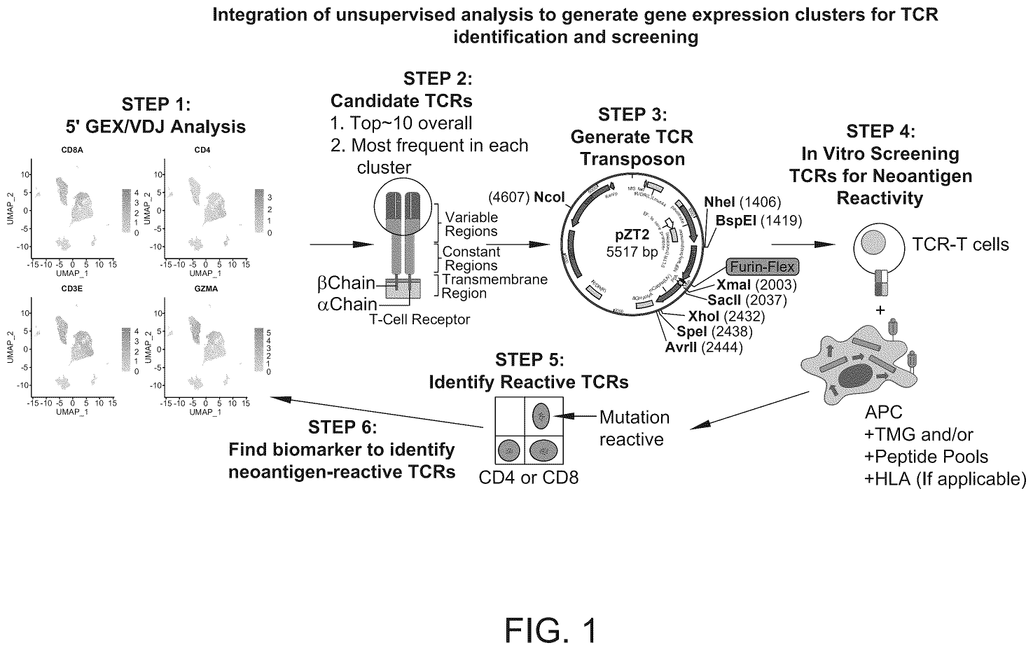

The present disclosure provides a TCR identification and screening platform as illustrated in FIG. 1. Initially, single-cell gene expression data (e.g., 5′ GEX Analysis) from T cells are utilized to perform unsupervised clustering analysis by employing dimensionality reduction methods such as principal component analysis (PCA), t-distributed Stochastic Neighbor Embedding (tSNE), or Uniform Manifold Approximation and Projection (UMAP) (FIG. 1, STEP 1). Merging the clustered single-cell gene expression analysis with paired, full-length TCR sequences then enables the identification of TCR clonotypes present in each of the distinct clusters. TCR sequences are then selected from the overall single-cell dataset based on frequency, cluster attributes, specific-gene expression signatures, or other criteria employed to increase the likelihood of obtaining TCRs with desired reactivity (i.e., antigen/HLA specificity) (FIG. 1, STEP 2). Selected paired, full-length TCR sequences are then reconstructed in silico, from which expression plasmids encoding the TCR α and β chains are synthesized (FIG. 1, STEP 3). These TCR expression cassettes are then cloned into transposon or other non-viral gene transfer vectors to enable quick translation into process development, manufacturing, and clinical applications. TCR-expression plasmids are then transiently expressed in a cell line (e.g., Jurkat or SUP-T1) or primary cell (e.g., human ex vivo expanded T cells) that will signal upon TCR recognition of cognate antigen:HLA complexes on the surface of antigen presenting cells (APCs) (FIG. 1, STEP 4). Antigen presenting cells (APCs) are classical professional APCs such as dendritic cells (DCs) or an artificial antigen presenting cell (e.g., COS-7 or 293-HEK). APCs either endogenously express the requisite HLA allele(s) or are transfected with HLA expression plasmids. Antigens are introduced to the APCs either by genetic transfer to antigen encoding plasmids (e.g., Tandem Minigene (TMG) plasmids) or by the pulsing of peptide pools. One aspect of the APC system used is that multiple HLA alleles and antigens are screened within the same set of APCs, thus enable high-throughput assessment of hundreds to thousands of antigen:HLA combinations. Co-culture of the TCR modified cells and APCs is then performed to identify reactive TCRs (FIG. 1, STEP 5). Reactive TCRs are those that are found to recognize one of the antigen:HLA conditions tested. These reactive TCRs are then further evaluated in vitro to confirm the findings and deconvolute the multiplexed HLA/antigen. Once all reactive TCRs are identified from a specimen, that binary outcome (reactive vs non-reactive) for each TCR can be mapped back to the initial gene-expression cluster analysis (FIG. 1, STEP 6). By mapping the reactive TCRs back to the gene-expression data, gene signatures or biomarkers which are enriched in the reactive TCR cell population are elucidated and used to further improve and refine the initial selection of TCRs for screening. In one aspect, this process is used to identify TCR sequences and their associated antigen and HLA specificity with a high level of confidence and accuracy from complex starting materials such as tumor tissues or blood samples.

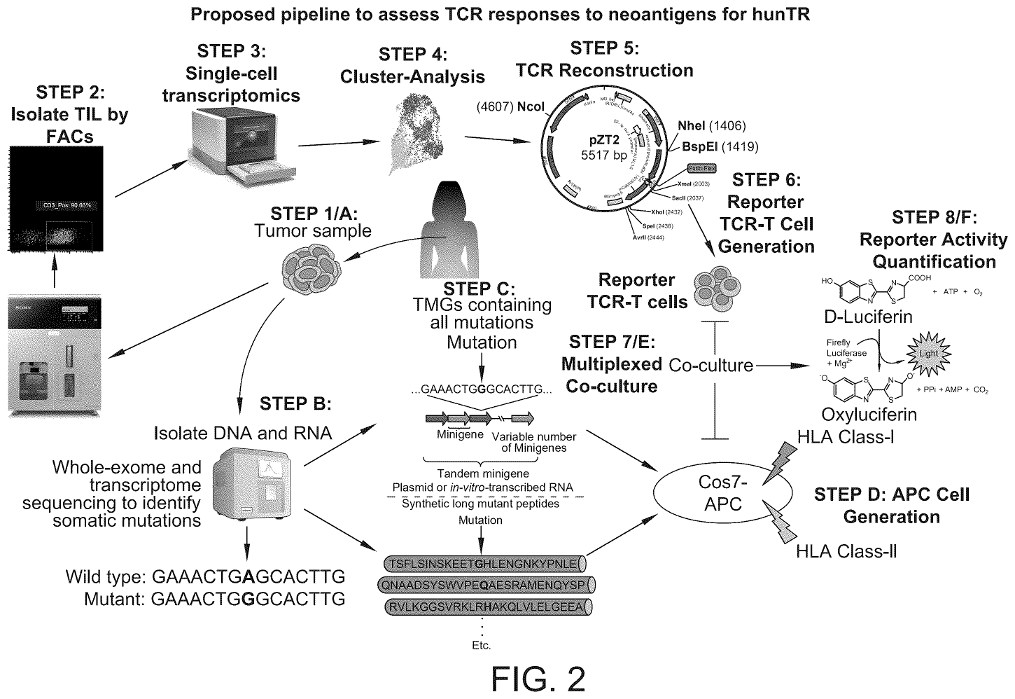

In one aspect, the steps of the above-described workflow (FIG. 1) comprise the processes as shown in FIG. 2 for screening of TCRs obtained from TILs. The process illustrated in FIG. 2 correspond to FIG. 1 STEPs 1-5. The workflow illustrated in FIG. 2 further comprises two parallel processes (indicated with either Alpha [i.e., A, B, C, etc.] or Numeric [i.e., 1, 2, 3, etc.] STEP designators) that diverge from a common starting point (STEP 1/A) and converge at a common finishing point (STEP 8/F). STEP 1/A to STEP 6 illustrate the workflow from TILs isolation to generation of cells expressing TILs-derived TCRs. STEP 1/A to STEP D illustrate the workflow from patient mutation and HLA calling to the generation of APCs expressing the patient matched HLA and mutation-derived antigens (e.g., neoantigens).

In one aspect, a tumor sample is obtained from a cancer patient (FIG. 2, STEP 1/A). This tumor sample is dissociated into a single-cell suspension and TILs are isolated by fluorescent activated cell sorting (FACS) by staining dissociated tumor samples for lymphocyte, T cell, and live cell markers (FIG. 2, STEP 2). Single-cell transcriptomics is then performed on the sorted TILs to obtain gene expression and TCR V(D)J sequences (FIG. 2, STEP 3). Bioinformatic analysis of the gene-expression data is used to cluster cells based on transcriptional similarities to aid in the selection of TCR sequences for in vitro evaluation (FIG. 2, STEP 4). Once selected, TCRs are reconstructed in silico and synthesized in expression vectors (FIG. 2, STEP 5) to enable transgenic expression of the TCRs in cells capable of forming a functional TCR complex with CD3 subunits and CD4/CD8 co-receptors. These cells are engineered to express any or all necessary protein components of the TCR signaling complex or downstream signaling components. Moreover, these components are modified to further enhance their function in the platform (e.g., CD4 with amino acid substitutions at Q40Y, T45W, P48L, S60R, and/or D63R to enhance affinity to MHC-Class II). Wang et al. 2011 PNAS, 108 (38):15960-15965. TCR expression vectors are transferred into the reporter cells to generate reporter TCR-T cells (FIG. 2 STEP 6).

In another aspect, in parallel to STEPs 1-6 described above, nucleic acids (DNA and RNA) are extracted from the tumor sample (FIG. 2, STEP 1/A). Using Whole Exome Sequencing (WES) and RNA Sequencing (RNAseq) to generate genomic and transcriptional datasets, a bioinformatics pipeline is employed to determine somatic mutations present in the tumor as well as the patient's germline HLA typing (FIG. 2, STEP B). Somatic mutations are ranked and concatenated so that TMGs and peptide pools can be synthesized (FIG. 2, STEP C). These reagents provide the antigen component of the screening assay. Similarly, sequences of the called HLA alleles are synthesized in expression vectors to provide the HLAs necessary for the screening assay. Antigen presenting cells, such as COS-7, are then modified either by stable or transient transfection to express the requisite Class I or Class II HLA alleles either in single-plex or multiplexed within the same cells (FIG. 2, STEP D). Antigen is provided to the APCs either by transfection of relevant TMGs (either as plasmid DNA or in vitro transcribed RNA) and/or peptide pools containing antigens derived from the tumor's somatic mutations identified. With both the HLA and antigen provided to the APCs, they are able to present peptide:HLA complexes to T cells in vitro.

In a further aspect, reporter cells expressing transgenic TCRs (FIG. 2, STEP 6) and antigen/HLA-modified APCs (FIG. 2, STEP D) are co-cultured together at a pre-determined ratio of Reporter cells (E) to APCs (T), typically approximately 4:1 to 8:1 (FIG. 2, STEP 7/E). Positive control wells containing PMA/Ionomycin or coated with H57-597 antibody (anti-transgenic TCR) with the TCR-modified Reporter cells are also set up. Negative control wells of Reporter cells alone or co-cultured with APCs modified with HLA-only, irrelevant antigens, or non-transfected are also set up. All conditions are typically evaluated in duplicate. After the co-culture period, reporter activity (i.e., luciferase activity) is quantified in each co-culture and control well (FIG. 2, STEP 8/F). For a given TCR, the reporter activity is compared across all antigen:HLA conditions evaluated to determine if there is a condition with increased reporter activity which indicates that the transgenic TCR recognized an antigen:HLA combination present in that well. Because initial screening multiplexes multiple HLA alleles and antigens, when there is specific TCR activity observed, STEP 7/E and 8/F are repeated using APCs modified with single HLA and antigens to elucidate the exact specificity of the TCR. Moreover, minimal epitopes can be determined using this co-culture method. Overall, this workflow enables the identification of TCR sequences and the empirical determination of specificity to selected antigens and HLA alleles.

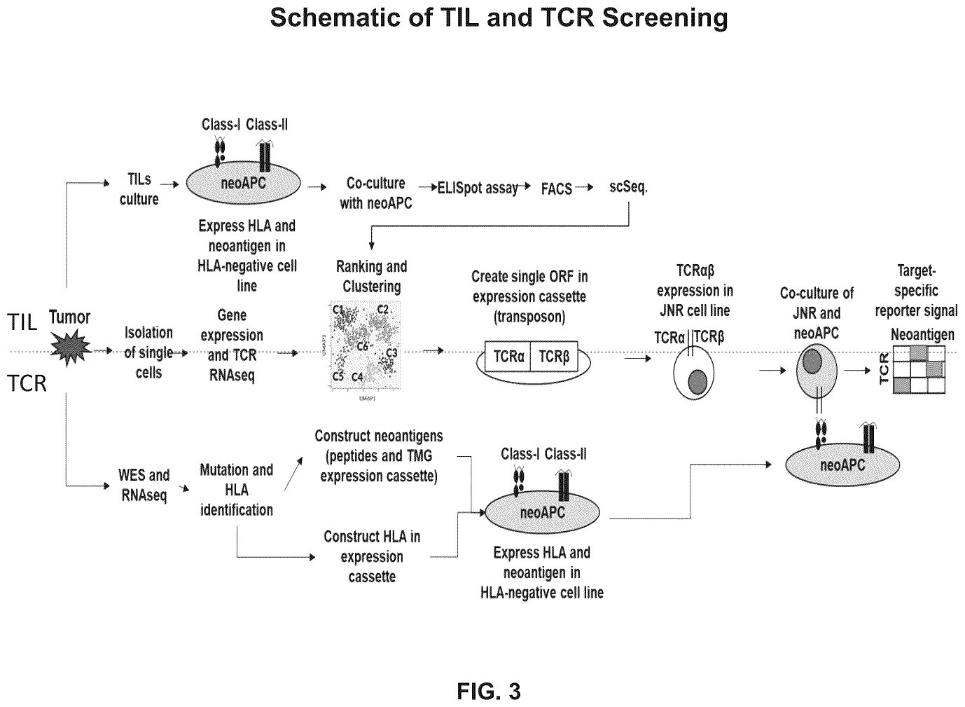

The present disclosure provides both a TCR-based screening method (below dotted line) and a TILs-based screening method (above dotted line), as illustrated in FIG. 3. The TCR-based screening method is as described above in the description of FIG. 2 wherein TCR sequences, somatic mutations, and HLA-typing is obtained from primary tumor samples and utilized to screen selected TCRs for reactivity to tumor neoantigens using a co-culture reporter system. Similarly, TILs screening starts with a primary tumor sample obtained from a cancer patient. TILs are expanded from the tumor using standard TILs expansion methods (high-concentration IL-2, feeder cells, muromonab-CD3 (OKT3)). Expanded TILs are then co-cultured in an IFN-γ ELISpot with APCs modified to express the relevant HLA alleles and antigens identified from WES and RNAseq data from the tumor. This is performed in a similar plate layout to TCR screening where multiple HLA alleles and antigens are multiplexed in the same wells, thus increasing the throughput of the assay. Positive controls include PMA/Ionomycin. Negative controls include TILs alone, APCs alone, TILs+APCs without HLA and/or antigen, and no cells. After the overnight co-culture, cells are harvested from the IFN-γ ELISpot and the plate is developed to measure the number of spot-forming colonies (SFCs) of each well. The harvested TILs are also stained and evaluated for upregulation of 4-1BB or other activation molecules (e.g., OX40). TILs from co-culture conditions which produce increased numbers of SFCs and/or activation marker expression are then sorted for either total live T cells or for T cells expressing the activation marker. Single cell gene expression and TCR V(D)J sequencing is then performed on the sorted cells. T cells from a negative control co-culture (typically APCs modified with HLA alone or with HLA and irrelevant antigen) are similarly sorted and analyzed by single-cell transcriptomics. Using the single-cell gene expression data, clusters of activated TILs can be identified. Paired, full-length TCR sequences from these activation clusters are then reconstructed into TCR expression plasmids and screened using the TCR screening methods described in FIG. 2. Overall, FIG. 3 illustrates parallel workflows with either ex vivo expanded TILs or sorted TILs are utilized to identify tumor-reactive TCRs with potential therapeutic applications in oncology. These general methods are applied to identify therapeutically useful TCRs in other disease indications (e.g., inflammation, auto-immune, etc.) with the appropriate starting material (e.g., a biopsy of inflamed colon from Crohn's disease patient or a plaque of a patient with psoriasis).

In one aspect, the cells in the methods or systems described herein are mammal cells, such as human cell, mouse cell, or monkey cells. In another aspect, the cells in the methods or systems described herein are non-human primate cells. In one aspect, the reporter T cells and the APCs are from different species.

In one aspect, the TCR expression cassette as disclosed herein comprises a TCR sequence reconstructed from TCR α and β chain sequences identified from TILs isolated from a tumor sample, and wherein the target neoantigen sequence and the matched HLA sequence are identified from the same tumor sample. Methods of identifying TCR sequences, antigen or neoantigen sequences, or the HLA sequences from a tumor sample or a normal reference sample are known in the art. Non-limiting examples of some commonly used methods are also disclosed herein. In one aspect, the TCR expression cassette is cloned into a non-viral gene transfer vector. In another aspect, the TCR expression cassette is cloned into a viral gene transfer vector. In a particular aspect, the non-viral gene transfer vector is a transposon.

In one aspect, the isolated TILs are first expanded ex vivo and then co-cultured with APCs modified to express relevant HLA alleles and antigens obtained from the tumor sample. In a further aspect, a gene signature for identifying neoantigen reactive TCRs from ex vivo expanded TILs includes one or more gene(s) selected from the group consisting of CSF2, NR4A3, TFNSF9, NR4A2, NR4A1, CRTAM, EGR2, DUSP2, XCL2, MYC, XCL1, TBC1D4, IFNG, TAGAP, TNF, RGCC, FABP5, SIAH2, PIM3, NAMPT, RAN, VSIR, ZBTB32, NOP16, ZBED2, DDX21, PGAM1, CCL3, HSPH1, CCL4, HSP90AB1, NOLC1, GADD45B, ATP1B3, PRDX1, NME1, and NPM1.

In one aspect, the reporter T cell disclosed herein is a primary T cell. In another aspect, the reporter T cell disclosed herein is from an immortalized T cell line. In a certain aspect, the reporter T cell disclosed herein is not a primary T cell. In certain aspects, the immortalized cell is a Jurkat cell or a SUP-T1 cell. In some aspects, the Jurkat cell is Jurkat NFAT. In one aspect, the endogenous T cell receptor of the cells is downregulated or knocked out, such as using routine methods in the art.

In one aspect, the reporter T cell disclosed herein expresses any or all protein components of the TCR signaling complex or downstream signaling components. In a certain aspect, the reporter T cell expresses one or more components selected from the group consisting of CD3, CD4, CD8a, and CD8b. In further aspects, these protein components are modified, such as by mutation of one or more amino acids, to enhance their activities.

In one aspect, the antigen presenting cell (APC) disclosed herein is a classical professional APC. In another aspect, the APCs disclosed herein are artificial APCs. In one aspect, the APC described herein does not express an endogenous human HLA. An endogenous human HLA may be knocked out from an APC by methods known in the art, e.g., CRISPR. In a further aspect, the APC comprises the machinery for antigen presentation still and be amenable to modification by transient or stable transgene expression of HLAs. In another aspect, the APC is modified with human beta-2-microglobulin, human CLIP, human TAP1 or TAP2, or any other human-derived molecular components of antigen processing and presentation. In a certain aspect, the APC disclosed herein is not a professional APC. In certain aspects, the APC used in the methods or cell systems disclosed herein is a COS cell. In one aspect, the COS cell is a COS-7 cell. In one aspect, the APC is a 293-HEK cell. In another aspect, the APC is not a 293-HEK cell. In one aspect, the APC endogenously expresses an HLA allele. In another aspect, the APC does not express any endogenous HLA. In one aspect, the APC comprises one or more HLA expression plasmids. In one aspect, the APC expresses multiple HLA alleles in a single cell.

In one aspect, the APC expresses a co-stimulatory molecule. Examples of the co-stimulatory molecules include, but not limited to, 4-1BBL, CD40, CD80, CD86, or OX40L.

In one aspect, antigen or neoantigen sequences are introduced to the APCs either by genetic transfer to antigen encoding plasmids (e.g., Tandem Minigene (TMG) plasmids) or by the pulsing of peptide pools. A Tandem Minigene is an open reading frame comprising concatenated minigenes which encode about 25 aa each. The minigenes encode the mutated region of the gene as identified from sequencing (typically 12 aa upstream and downstream of the substituted aa residue). These minigenes are flanked at the 5′ end with a LAMP1 signal peptide and 3′ end DC-LAMP localization signal. One aspect of the APC system used is that multiple HLA alleles and antigens are screened within the same set of APCs, thus enable high-throughput assessment of hundreds to thousands of antigen:HLA combinations. In one aspect, a “matched” HLA sequence of a neoantigen sequence refers to an HLA sequence that is identified from tissue, blood, or tumor samples of the same patient as the TCR sequence and neoantigen sequence. In certain aspect, “matched” HLA sequence may also be used to indicate the HLA sequence of the HLA allele for which a particular TCR is restricted.

In some aspects, the reporter T cell disclosed herein comprises a reporter system that is activated by the binding of a TCR to an antigen. Examples of the reporter systems are known in the art and include, but are not limited to, systems based on luciferase activity, fluorescence, or cytokine production.

In one aspect of the present disclosure, the reporter T cells and the APCs are co-cultured at a ratio from about 16:1 to about 1:16. In one aspect, the reporter T cells and the APCs are co-cultured at a ratio of about 4:1. In another aspect, the reporter T cells and the APCs are co-cultured at a ratio of about 8:1.

In one aspect, the reporter T cells and the APCs are co-cultured for 1 to 48 hours. In one aspect, the reporter T cells and the APCs are co-cultured for at least one hour, at least 2 hours, at least 3 hours, at least 4 hours, at least 5 hours, at least 6 hours, at least 7 hours, at least 8 hours, at least 9 hours, or at least 10 hours. In another aspect, the reporter T cells and the APCs are co-cultured for about one hour, about 2 hours, about 3 hours, about hours, at least 5 hours, about 6 hours, about 7 hours, about 8 hours, about 9 hours, or about 10 hours.

In one aspect, the TCRs disclosed herein interacts with and/or is specific for a peptide from a gene selected from a group comprising KRAS, RHPN2, GFRA2, NUP205, PCSK9, CEP85, HNRNPF, KDMIA, USP9X, LLGL1, ACO2, POLDIP3, EMC8, LCK, RCC1, VARS, LCK, ATP1A1, and CRYBG3.

The present disclosure provides TCR sequences, or an antigen-binding portion thereof, that are identified or obtained by any of the methods disclosed herein. In one aspect, a TCR sequence comprises one or more of the sequences selected from the group consisting of SEQ ID NOs: 1-300, 536-1003, and 1025-1204 (the sequences provided in Tables 1-79). In another aspect, a TCR sequence comprises a sequence that is at least 80%, at least 85%, at least 90%, or at least 95% identical to a sequence selected from the group consisting of SEQ ID NOs: 1-300, 536-1003, and 1025-1204 (the sequences provided in Tables 1-79).

In Tables 1 to 79, all of the sequences are fully human except for the “α chain with WT signal peptide and constant Cα” and “β chain with WT signal peptide and constant Cβ.” The sequences in these two sections are chimeric, containing the variable region sequences of the human TCRs combined with the constant region sequences of murine a and β chains.

TABLE 1

| SEQ ID | | |
|---|---|---|
| NO. | Description | 2599-TCR12 |
| 1 | CDR1α | VTNFRS |
| 2 | CDR2α | LTSSGIE |
| 3 | CDR3α | GGLNAGGTSYGKLT |
| 4 | Vα without signal | EDKVVQSPLSLVVHEGDTVTLNCSYEVTNERSLLW |
| | peptide (SignalP) | YKQEKKAPTFLFMLTSSGIEKKSGRLSSILDKKEL |
| | | FSILNITATQTGDSAIYLCGGLNAGGTSYGKLTFG |
| | | QGTILTVHP |
| 5 | Vα only (without the | MMKCPQALLAIFWLLLSWVSSEDKVVQSPLSLVVH |
| | Constant) | EGDTVTLNCSYEVTNFRSLLWYKQEKKAPTELFML |
| | | TSSGIEKKSGRLSSILDKKELFSILNITATQTGDS |
| | | AIYLCGGLNAGGTSYGKLTFGQGTILTVHP |
| 6 | α chain with WT signal | MMKCPQALLAIFWLLLSWVSSEDKVVQSPLSLVVH |
| | peptide and constant Cα | EGDTVTLNCSYEVINFRSLLWYKQEKKAPTFLEML |
| | | TSSGIEKKSGRLSSILDKKELFSILNITATQTGDS |
| | | AIYLCGGLNAGGTSYGKLTFGQGTILTVHPNIQNP |
| | | EPAVYQLKDPRSQDSTLCLFTDFDSQINVPKTMES |
| | | GTFITDKTVLDMKAMDSKSNGAIAWSNQTSFTCQD |
| | | IFKETNATYPSSDVPCDATLTEKSFETDMNLNFQN |
| | | LLVIVLRILLLKVAGFNLLMTLRLWSS |
| 7 | CDR1β | SNHLY |
| 8 | CDR2β | FYNNEI |
| 9 | CDR3β | ASLGASTYEQY |
| 10 | Vβ without signal | EPEVTQTPSHQVTQMGQEVILRCVPISNHLYFYWY |
| | peptide (SignalP) | RQILGQKVEFLVSFYNNEISEKSEIFDDQFSVERP |
| | | DGSNFTLKIRSTKLEDSAMYFCASLGASTYEQYFG |
| | | PGTRLTVT |
| 11 | Vβ (without the Constant) | MDTWLVCWAIFSLLKAGLTEPEVTQTPSHQVTQMG |
| | | QEVILRCVPISNHLYFYWYRQILGQKVEFLVSFYN |
| | | NEISEKSEIFDDQFSVERPDGSNFTLKIRSTKLED |
| | | SAMYFCASLGASTYEQYFGPGTRLTVT |
| 12 | β chain with WT signal | MDTWLVCWAIFSLLKAGLTEPEVTQTPSHQVTQMG |
| | peptide and constant Cβ | QEVILRCVPISNHLYFYWYRQILGQKVEFLVSFYN |
| | | NEISEKSEIFDDQFSVERPDGSNFTLKIRSTKLED |
| | | SAMYFCASLGASTYEQYFGPGTRLTVTEDLRNVTP |
| | | PKVSLFEPSKAEIANKQKATLVCLARGFFPDHVEL |
| | | SWWVNGKEVHSGVSTDPQAYKESNYSYCLSSRLRV |
| | | SATFWHNPRNHFRCQVQFHGLSEEDKWPEGSPKPV |
| | | TQNISAEAWGRADCGITSASYQQGVLSATILYEIL |
| | | LGKATLYAVLVSTLVVMAMVKRKNS |

In some embodiments, 2599-TCR12 interacts with and/or is specific for a peptide from gene ERGIC2. In some embodiments, the peptide is from a neoantigen of ERGIC2 and has the amino acid change L176P (in which position 176 of the ERGIC2 protein is mutated from Leu to Pro). In some embodiments, 2599-TCR12 interacts with and/or is specific for the neoantigen in the context of HLA-A*03:01.

TABLE 2

| SEQ ID | | |
|---|---|---|
| NO. | Description | 6932-TCR5 |
| 73 | CDR1α | NSASQS |
| 74 | CDR2α | VYSSGN |
| 75 | CDR3α | VVKNQGGKLI |
| 76 | Vα without signal peptide | QRKEVEQDPGPFNVPEGATVAFNCTYSNSASQS |
| | (SignalP) | FFWYRQDCRKEPKLLMSVYSSGNEDGRFTAQLN |
| | | RASQYISLLIRDSKLSDSATYLCVVKNQGGKLI |
| | | FGQGTELSVKP |
| 77 | Vα only (without the | MISLRVLLVILWLQLSWVWSQRKEVEQDPGPEN |
| | Constant) | VPEGATVAFNCTYSNSASQSFFWYRQDCRKEPK |
| | | LLMSVYSSGNEDGRFTAQLNRASQYISLLIRDS |
| | | KLSDSATYLCVVKNQGGKLIFGQGTELSVKP |
| 78 | α chain with WT signal | MISLRVLLVILWLQLSWVWSQRKEVEQDPGPEN |
| | peptide and constant Cα | VPEGATVAFNCTYSNSASQSFFWYRQDCRKEPK |
| | | LLMSVYSSGNEDGRFTAQLNRASQYISLLIRDS |
| | | KLSDSATYLCVVKNQGGKLIFGQGTELSVKPNI |
| | | QNPEPAVYQLKDPRSQDSTLCLFTDFDSQINVP |
| | | KTMESGTFITDKTVLDMKAMDSKSNGAIAWSNQ |
| | | TSFTCQDIFKETNATYPSSDVPCDATLTEKSFE |
| | | TDMNLNFQNLLVIVLRILLLKVAGFNLLMTLRL |
| | | WSS |
| 79 | CDR1β | SGHRS |
| 80 | CDR2β | YFSETQ |
| 81 | CDR3β | ASILGGGRGDTQY |
| 82 | Vβ without signal peptide | GVTQTPRYLIKTRGQQVTLSCSPISGHRSVSWY |
| | (SignalP) | QQTPGQGLQFLFEYFSETQRNKGNFPGRFSGRQ |
| | | FSNSRSEMNVSTLELGDSALYLCASILGGGRGD |
| | | TQYFGPGTRLTVL |
| 83 | Vβ (without the Constant) | MGSRLLCWVLLCLLGAGPVKAGVTQTPRYLIKT |
| | | RGQQVTLSCSPISGHRSVSWYQQTPGQGLQFLF |
| | | EYFSETQRNKGNFPGRFSGRQFSNSRSEMNVST |
| | | LELGDSALYLCASILGGGRGDTQYFGPGTRLTV |
| 84 | β chain with WT signal | MGSRLLCWVLLCLLGAGPVKAGVTQTPRYLIKT |
| | peptide and constant Cβ | RGQQVTLSCSPISGHRSVSWYQQTPGQGLQFLF |
| | | EYFSETQRNKGNFPGRFSGRQFSNSRSEMNVST |
| | | LELGDSALYLCASILGGGRGDTQYFGPGTRLTV |
| | | LEDLRNVTPPKVSLFEPSKAEIANKQKATLVCL |
| | | ARGFFPDHVELSWWVNGKEVHSGVSTDPQAYKE |
| | | SNYSYCLSSRLRVSATFWHNPRNHFRCQVQFHG |
| | | LSEEDKWPEGSPKPVTQNISAEAWGRADCGITS |
| | | ASYQQGVLSATILYEILLGKATLYAVLVSTLVV |
| | | MAMVKRKNS |

In some embodiments, 6932-TCR5 interacts with and/or is specific for a peptide from gene HELZ2. In some embodiments, the peptide is from a neoantigen of HELZ2 and has the amino acid change P775A (in which position 775 of the HELZ2 protein is mutated from Pro to Ala). In some embodiments, 6932-TCR5 interacts with and/or is specific for the neoantigen in the context of DPA1*01:03; HLA-DPB1*104:01 and/or HLA-DPA1*03:01; HLA-DPB1*104:01.

TABLE 3

| SEQ ID NO. | Description | 8434-TCR3 |
|---|---|---|
| 13 | CDR1α | VSGNPY |
| 14 | CDR2α | YITGDNLV |
| 15 | CDR3α | AVRDDYGQNFV |
| 16 | Vα without signal | DTGVSQNPRHKITKRGQNVTFRCDPISEHNRLYWYRQTL |
| | peptide (SignalP) | GQGPEFLTYFQNEAQLEKSRLLSDRFSAERPKGSFSTLE |
| | | IQRTEQGDSAMYLCASSLSGPSYEQYFGPGTRLTVT |
| 17 | Vα only (without | MGTSLLCWMALCLLGADHADTGVSQNPRHKITKRGQNVT |
| | the Constant) | FRCDPISEHNRLYWYRQTLGQGPEFLTYFQNEAQLEKSR |
| | | LLSDRFSAERPKGSFSTLEIQRTEQGDSAMYLCASSLSG |
| | | PSYEQYFGPGTRLTVT |
| 18 | α chain with WT | MGTSLLCWMALCLLGADHADTGVSQNPRHKITKRGQNVT |
| | signal peptide and | FRCDPISEHNRLYWYRQTLGQGPEFLTYFQNEAQLEKSR |
| | constant Cα | LLSDRFSAERPKGSESTLEIQRTEQGDSAMYLCASSLSG |
| | | PSYEQYFGPGTRLTVTNIQNPEPAVYQLKDPRSQDSTLC |
| | | LFTDFDSQINVPKTMESGTFITDKTVLDMKAMDSKSNGA |
| | | IAWSNQTSFTCQDIFKETNATYPSSDVPCDATLTEKSFE |
| | | TDMNLNFQNLLVIVLRILLLKVAGFNLLMTLRLWSS |
| 19 | CDR1β | SEHNR |
| 20 | CDR2β | FQNEAQ |
| 21 | CDR3β | ASSLSGPSYEQY |
| 22 | Vβ without signal | QSVAQPEDQVNVAEGNPLTVKCTYSVSGNPYLFWYVQYP |
| | peptide (SignalP) | NRGLQFLLKYITGDNLVKGSYGFEAEFNKSQTSFHLKKP |
| | | SALVSDSALYFCAVRDDYGQNFVFGPGTRLSVLP |
| 23 | Vβ (without the | MASAPISMLAMLFTLSGLRAQSVAQPEDQVNVAEGNPLT |
| | Constant) | VKCTYSVSGNPYLFWYVQYPNRGLQFLLKYITGDNLVKG |
| | | SYGFEAEENKSQTSFHLKKPSALVSDSALYFCAVRDDYG |
| | | QNFVFGPGTRLSVLP |
| 24 | β chain with WT | MASAPISMLAMLFTLSGLRAQSVAQPEDQVNVAEGNPLT |
| | signal peptide and | VKCTYSVSGNPYLFWYVQYPNRGLQFLLKYITGDNLVKG |
| | constant Cβ | SYGFEAEFNKSQTSFHLKKPSALVSDSALYFCAVRDDYG |
| | | QNFVFGPGIRLSVLPEDLRNVTPPKVSLFEPSKAEIANK |
| | | QKATLVCLARGFFPDHVELSWWVNGKEVHSGVSTDPQAY |
| | | KESNYSYCLSSRLRVSATFWHNPRNHFRCQVQFHGLSEE |
| | | DKWPEGSPKPVTQNISAEAWGRADCGITSASYQQGVLSA |
| | | TILYEILLGKATLYAVLVSTLVVMAMVKRKNS |

In some embodiments, 8434-TCR3 interacts with and/or is specific for a peptide from the KRAS. In some embodiments, the peptide is from a neoantigen of KRAS and has the amino acid change Q61H (in which position 61 of the KRAS protein is mutated from Gln to His). In some embodiments, 8434-TCR3 interacts with and/or is specific for the neoantigen in the context of HLA-B*35:02.

TABLE 4

| SEQ ID NO. | Description | 8434-TCR20 |
|---|---|---|
| 25 | CDR1α | DSAIYN |
| 26 | CDR2α | IQSSQRE |
| 27 | CDR3α | AVRHSGNTPLV |
| 28 | Vα without signal | KQEVTQIPAALSVPEGENLVLNCSFTDSAIYNLQWFRQD |
| | peptide (SignalP) | PGKGLTSLLLIQSSQREQTSGRLNASLDKSSGRSTLYIA |
| | | ASQPGDSATYLCAVRHSGNTPLVFGKGIRLSVIA |
| 29 | Vα only (without | METLLGLLILWLQLQWVSSKQEVTQIPAALSVPEGENLV |
| | the Constant) | LNCSFTDSAIYNLQWFRQDPGKGLTSLLLIQSSQREQTS |
| | | GRLNASLDKSSGRSTLYIAASQPGDSATYLCAVRHSGNT |
| | | PLVFGKGTRLSVIA |
| 30 | α chain with WT | METLLGLLILWLQLQWVSSKQEVTQIPAALSVPEGENLV |
| | signal peptide and | LNCSFTDSAIYNLQWFRQDPGKGLTSLLLIQSSQREQTS |
| | constant Cα | GRLNASLDKSSGRSTLYIAASQPGDSATYLCAVRHSGNT |
| | | PLVFGKGTRLSVIANIQNPEPAVYQLKDPRSQDSTLCLF |
| | | TDFDSQINVPKTMESGTFITDKTVLDMKAMDSKSNGAIA |
| | | WSNQTSFTCQDIFKETNATYPSSDVPCDATLTEKSFETD |
| | | MNLNFQNLLVIVLRILLLKVAGFNLLMTLRLWSS |
| 31 | CDR1β | DFQATT |
| 32 | CDR2β | SNEGSKA |
| 33 | CDR3β | SASGGGRTEAF |
| 34 | Vβ without signal | AVVSQHPSRVICKSGTSVKIECRSLDFQATTMFWYRQFP |
| | peptide (SignalP) | KQSLMLMATSNEGSKATYEQGVEKDKFLINHASLTLSTL |
| | | TVTSAHPEDSSFYICSASGGGRTEAFFGQGTRLTVV |
| 35 | Vβ (without the | MLLLLLLLGPGSGLGAVVSQHPSRVICKSGTSVKIECRS |
| | Constant) | LDFQATTMFWYRQFPKQSLMLMATSNEGSKATYEQGVEK |
| | | DKFLINHASLTLSTLTVTSAHPEDSSFYICSASGGGRTE |
| | | AFFGQGTRLTVV |
| 36 | β chain with WT | MLLLLLLLGPGSGLGAVVSQHPSRVICKSGTSVKIECRS |
| | signal peptide and | LDFQATTMFWYRQFPKQSLMLMATSNEGSKATYEQGVEK |
| | constant Cβ | DKFLINHASLTLSTLTVTSAHPEDSSFYICSASGGGRTE |
| | | AFFGQGTRLTVVEDLRNVTPPKVSLFEPSKAEIANKQKA |
| | | TLVCLARGFFPDHVELSWWVNGKEVHSGVSTDPQAYKES |
| | | NYSYCLSSRLRVSATFWHNPRNHFRCQVQFHGLSEEDKW |
| | | PEGSPKPVTQNISAEAWGRADCGITSASYQQGVLSATIL |
| | | YEILLGKATLYAVLVSTLVVMAMVKRKNS |

In some embodiments, 8434-TCR20 interacts with and/or is specific for a peptide from the protein encoded by the ARHGEF16 gene. In some embodiments, the peptide is from a neoantigen of the protein encoded by the ARHGEF16 gene and has the amino acid change p.R150W (in which position 150 of the protein encoded by the ARHGEF16 gene is mutated from Arg to Trp). In some embodiments, 8434-TCR20 interacts with and/or is specific for the neoantigen in the context of HLA-DRA and DRB1*11:01.

TABLE 5

| SEQ ID NO. | Description | 8434-TCR21 |
|---|---|---|
| 37 | CDR1α | DSAIYN |
| 38 | CDR2α | IQSSQRE |
| 39 | CDR3α | AVRRDGTASKLT |
| 40 | Vα without signal | KQEVTQIPAALSVPEGENLVLNCSFTDSAIYNLQWFRQD |
| | peptide (SignalP) | PGKGLTSLLLIQSSQREQTSGRLNASLDKSSGRSTLYIA |
| | | ASQPGDSATYLCAVRRDGTASKLTFGTGTRLQVTL |
| 41 | Vα only (without | METLLGLLILWLQLQWVSSKQEVTQIPAALSVPEGENLV |
| | the Constant) | LNCSFTDSAIYNLQWFRQDPGKGLTSLLLIQSSQREQTS |
| | | GRLNASLDKSSGRSTLYIAASQPGDSATYLCAVRRDGTA |
| | | SKLTFGTGTRLQVTL |
| 42 | α chain with WT | METLLGLLILWLQLQWVSSKQEVTQIPAALSVPEGENLV |
| | signal peptide and | LNCSFTDSAIYNLQWFRQDPGKGLTSLLLIQSSQREQTS |
| | constant Cα | GRLNASLDKSSGRSTLYIAASQPGDSATYLCAVRRDGTA |
| | | SKLTFGTGTRLQVTLNIQNPEPAVYQLKDPRSQDSTLCL |
| | | FTDFDSQINVPKTMESGTFITDKTVLDMKAMDSKSNGAI |
| | | AWSNQTSFTCQDIFKETNATYPSSDVPCDATLTEKSFET |
| | | DMNLNFQNLLVIVLRILLLKVAGFNLLMTLRLWSS |
| 43 | CDR1β | DFQATT |
| 44 | CDR2β | SNEGSKA |
| 45 | CDR3β | SASFPGRGNEQF |
| 46 | Vβ without signal | AVVSQHPSWVICKSGTSVKIECRSLDFQATTMFWYRQFP |
| | peptide (SignalP) | KQSLMLMATSNEGSKATYEQGVEKDKFLINHASLTLSTL |
| | | TVTSAHPEDSSFYICSASFPGRGNEQFFGPGTRLTVL |
| 47 | Vβ (without the | MLLLLLLLGPGSGLGAVVSQHPSWVICKSGTSVKIECRS |
| | Constant) | LDFQATTMFWYRQFPKQSLMLMATSNEGSKATYEQGVEK |
| | | DKFLINHASLTLSTLTVTSAHPEDSSFYICSASFPGRGN |
| | | EQFFGPGTRLTVL |
| 48 | β chain with WT | MLLLLLLLGPGSGLGAVVSQHPSWVICKSGTSVKIECRS |
| | signal peptide and | LDFQATTMFWYRQFPKQSLMLMATSNEGSKATYEQGVEK |
| | constant Cβ | DKFLINHASLTLSTLTVTSAHPEDSSFYICSASFPGRGN |
| | | EQFFGPGTRLTVLEDLRNVTPPKVSLFEPSKAEIANKQK |
| | | ATLVCLARGFFPDHVELSWWVNGKEVHSGVSTDPQAYKE |
| | | SNYSYCLSSRLRVSATFWHNPRNHFRCQVQFHGLSEEDK |
| | | WPEGSPKPVTQNISAEAWGRADCGITSASYQQGVLSATI |
| | | LYEILLGKATLYAVLVSTLVVMAMVKRKNS |

In some embodiments, 8434-TCR21 interacts with and/or is specific for a peptide from the protein encoded by the ARHGEF16 gene. In some embodiments, the peptide is from a neoantigen of the protein encoded by the ARHGEF16 gene and has the amino acid change p.R150W (in which position 150 of the protein encoded by the ARHGEF16 gene is mutated from Arg to Trp). In some embodiments, 8434-TCR21 interacts with and/or is specific for the neoantigen in the context of HLA-DRA and DRB1*11:01.

TABLE 6

| SEQ ID NO. | Description | 8434-TCR23 |
|---|---|---|
| 49 | CDR1α | NTAFDY |
| 50 | CDR2α | IRPDVSE |
| 51 | CDR3α | AASRASGGSYIPT |
| 52 | Vα without signal | QQKEKSDQQQVKQSPQSLIVQKGGISIINCAYENTAFDYF |
| | peptide (SignalP) | PWYQQFPGKGPALLIAIRPDVSEKKEGRFTISFNKSAKQF |
| | | SLHIMDSQPGDSATYFCAASRASGGSYIPTFGRGTSLIVH |
| | | P |
| 53 | Vα only (without | MDKILGASFLVLWLQLCWVSGQQKEKSDQQQVKQSPQSLI |
| | the Constant) | VQKGGISIINCAYENTAFDYFPWYQQFPGKGPALLIAIRP |
| | | DVSEKKEGRFTISFNKSAKQFSLHIMDSQPGDSATYFCAA |
| | | SRASGGSYIPTFGRGTSLIVHP |
| 54 | α chain with WT | MDKILGASFLVLWLQLCWVSGQQKEKSDQQQVKQSPQSLI |
| | signal peptide and | VQKGGISIINCAYENTAFDYFPWYQQFPGKGPALLIAIRP |
| | constant Cα | DVSEKKEGRFTISFNKSAKQFSLHIMDSQPGDSATYFCAA |
| | | SRASGGSYIPTFGRGTSLIVHPNIQNPEPAVYQLKDPRSQ |
| | | DSTLCLFTDFDSQINVPKTMESGTFITDKTVLDMKAMDSK |
| | | SNGAIAWSNQTSFTCQDIFKETNATYPSSDVPCDATLTEK |
| | | SFETDMNLNFQNLLVIVLRILLLKVAGFNLLMTLRLWSS |
| 55 | CDR1β | LNHDA |
| 56 | CDR2β | SQIVND |
| 57 | CDR3β | ATKILAGANTGELF |
| 58 | Vβ without signal | GITQSPKYLFRKEGQNVTLSCEQNLNHDAMYWYRQDPGQG |
| | peptide (SignalP) | LRLIYYSQIVNDFQKGDIAEGYSVSREKKESFPLTVTSAQ |
| | | KNPTAFYLCATKILAGANTGELFFGEGSRLTVL |
| 59 | Vβ (without the | MSNQVLCCVVLCLLGANTVDGGITQSPKYLFRKEGQNVTL |
| | Constant) | SCEQNLNHDAMYWYRQDPGQGLRLIYYSQIVNDFQKGDIA |
| | | EGYSVSREKKESFPLTVTSAQKNPTAFYLCATKILAGANT |
| | | GELFFGEGSRLTVL |
| 60 | β chain with WT | MSNQVLCCVVLCLLGANTVDGGITQSPKYLFRKEGQNVTL |
| | signal peptide and | SCEQNLNHDAMYWYRQDPGQGLRLIYYSQIVNDFQKGDIA |
| | constant Cβ | EGYSVSREKKESFPLTVTSAQKNPTAFYLCATKILAGANT |
| | | GELFFGEGSRLTVLEDLRNVTPPKVSLFEPSKAEIANKQK |
| | | ATLVCLARGFFPDHVELSWWVNGKEVHSGVSTDPQAYKES |
| | | NYSYCLSSRLRVSATFWHNPRNHFRCQVQFHGLSEEDKWP |
| | | EGSPKPVTQNISAEAWGRADCGITSASYQQGVLSATILYE |
| | | ILLGKATLYAVLVSTLVVMAMVKRKNS |

In some embodiments, 8434-TCR23 interacts with and/or is specific for a peptide from the protein encoded by the ARHGEF16 gene. In some embodiments, the peptide is from a neoantigen of the protein encoded by the ARHGEF16 gene and has the amino acid change p.R150W (in which position 150 of the protein encoded by the ARHGEF16 gene is mutated from Arg to Trp). In some embodiments, 8434-TCR23 interacts with and/or is specific for the neoantigen in the context of DRB1*11:01.

TABLE 7

| SEQ ID NO. | Description | 8434-TCR27 |
|---|---|---|
| 61 | CDR1α | VGISA |
| 62 | CDR2α | LSSGK |
| 63 | CDR3α | AAGGPQLAPKETSGSRLT |
| 64 | Vα without signal | AKNEVEQSPQNLTAQEGEFITINCSYSVGISALHWLQQHP |
| | peptide (SignalP) | GGGIVSLFMLSSGKKKHGRLIATINIQEKHSSLHITASHP |
| | | RDSAVYICAAGGPQLAPKETSGSRLTFGEGTQLTVNP |
| 65 | Vα only (without | MVKIRQFLLAILWLQLSCVSAAKNEVEQSPQNLTAQEGEF |
| | the Constant) | ITINCSYSVGISALHWLQQHPGGGIVSLFMLSSGKKKHGR |
| | | LIATINIQEKHSSLHITASHPRDSAVYICAAGGPQLAPKE |
| | | TSGSRLTFGEGTQLTVNP |
| 66 | α chain with WT | MVKIRQFLLAILWLQLSCVSAAKNEVEQSPQNLTAQEGEF |
| | signal peptide and | ITINCSYSVGISALHWLQQHPGGGIVSLEMLSSGKKKHGR |
| | constant Cα | LIATINIQEKHSSLHITASHPRDSAVYICAAGGPQLAPKE |
| | | TSGSRLTFGEGTQLTVNPNIQNPEPAVYQLKDPRSQDSTL |
| | | CLFTDFDSQINVPKTMESGTFITDKTVLDMKAMDSKSNGA |
| | | IAWSNQTSFTCQDIFKETNATYPSSDVPCDATLTEKSFET |
| | | DMNLNFQNLLVIVLRILLLKVAGFNLLMTLRLWSS |
| 67 | CDR1β | SGHRS |
| 68 | CDR2β | YFSETQ |
| 69 | CDR3β | ASSLDPSGTGYT |
| 70 | Vβ without signal | GVTQTPRYLIKTRGQQVTLSCSPISGHRSVSWYQQTPGQG |
| | peptide (SignalP) | LQFLFEYFSETQRNKGNFPGRFSGRQFSNSRSEMNVSTLE |
| | | LGDSALYLCASSLDPSGTGYTFGSGTRLTVV |
| 71 | Vβ (without the | MGSRLLCWVLLCLLGAGPVKAGVTQTPRYLIKTRGQQVTL |
| | Constant) | SCSPISGHRSVSWYQQTPGQGLQFLFEYFSETQRNKGNFP |
| | | GRFSGRQFSNSRSEMNVSTLELGDSALYLCASSLDPSGTG |
| | | YTFGSGTRLTVV |
| 72 | β chain with WT | MGSRLLCWVLLCLLGAGPVKAGVTQTPRYLIKTRGQQVTL |
| | signal peptide and | SCSPISGHRSVSWYQQTPGQGLQFLFEYFSETQRNKGNFP |
| | constant Cβ | GRFSGRQFSNSRSEMNVSTLELGDSALYLCASSLDPSGTG |
| | | YTFGSGTRLTVVEDLRNVTPPKVSLFEPSKAEIANKQKAT |
| | | LVCLARGFFPDHVELSWWVNGKEVHSGVSTDPQAYKESNY |
| | | SYCLSSRLRVSATFWHNPRNHFRCQVQFHGLSEEDKWPEG |
| | | SPKPVTQNISAEAWGRADCGITSASYQQGVLSATILYEIL |
| | | LGKATLYAVLVSTLVVMAMVKRKNS |

In some embodiments, 8434-TCR27 interacts with and/or is specific for a peptide from the KRAS protein. In some embodiments, the peptide is from a neoantigen of KRAS and has the amino acid change p.Q61H (in which position 61 of the KRAS protein is mutated from Gln to His). In some embodiments, 8434-TCR27 interacts with and/or is specific for the neoantigen in the context of HLA-B*35:02.

TABLE 8

| SEQ ID NO. | Description | 0025-TCR8 |
|---|---|---|
| 85 | CDR1α | SVESS |
| 86 | CDR2α | VVTGGEV |
| 87 | CDR3α | AGDRPGNTPLV |
| 88 | Vα without signal | QLLEQSPQFLSIQEGENLTVYCNSSSVESSLQWYRQEPG |
| | peptide (SignalP) | EGPVLLVTVVTGGEVKKLKRLTFQFGDARKDSSLHITAA |
| | | QPGDTGLYLCAGDRPGNTPLVFGKGTRLSVIA |
| 89 | Vα only (without | MVLKFSVSILWIQLAWVSTQLLEQSPQFLSIQEGENLTV |
| | the Constant) | YCNSSSVFSSLQWYRQEPGEGPVLLVTVVTGGEVKKLKR |
| | | LTFQFGDARKDSSLHITAAQPGDTGLYLCAGDRPGNTPL |
| | | VFGKGTRLSVIA |
| 90 | α chain with WT | MVLKFSVSILWIQLAWVSTQLLEQSPQFLSIQEGENLTV |
| | signal peptide and | YCNSSSVFSSLQWYRQEPGEGPVLLVTVVTGGEVKKLKR |
| | constant Cα | LTFQFGDARKDSSLHITAAQPGDTGLYLCAGDRPGNTPL |
| | | VFGKGTRLSVIA |
| 91 | CDR1β | SGHTA |
| 92 | CDR2β | FQGNSA |
| 93 | CDR3β | ASSLSQGSSYEQY |
| 94 | Vβ without signal | GVSQSPSNKVTEKGKDVELRCDPISGHTALYWYRQSLGQ |
| | peptide (SignalP) | GLEFLIYFQGNSAPDKSGLPSDRFSAERTGGSVSTLTIQ |
| | | RTQQEDSAVYLCASSLSQGSSYEQYFGPGTRLTVT |
| 95 | Vβ (without the | MGTRLLFWVAFCLLGADHTGAGVSQSPSNKVTEKGKDVE |
| | Constant) | LRCDPISGHTALYWYRQSLGQGLEFLIYFQGNSAPDKSG |
| | | LPSDRFSAERTGGSVSTLTIQRTQQEDSAVYLCASSLSQ |
| | | GSSYEQYFGPGTRLTVT |
| 96 | β chain with WT | MGTRLLFWVAFCLLGADHTGAGVSQSPSNKVTEKGKDVE |
| | signal peptide and | LRCDPISGHTALYWYRQSLGQGLEFLIYFQGNSAPDKSG |
| | constant Cβ | LPSDRFSAERTGGSVSTLTIQRTQQEDSAVYLCASSLSQ |
| | | GSSYEQYFGPGTRLTVTEDLRNVTPPKVSLFEPSKAEIA |
| | | NKQKATLVCLARGFFPDHVELSWWVNGKEVHSGVSTDPQ |
| | | AYKESNYSYCLSSRLRVSATFWHNPRNHFRCQVQFHGLS |
| | | EEDKWPEGSPKPVTQNISAEAWGRADCGITSASYQQGVL |
| | | SATILYEILLGKATLYAVLVSTLVVMAMVKRKNS |

In some embodiments, 0025-TCR8 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR8 interacts with and/or is specific for the neoantigen in the context of HLA-DRA and DRB1*01:01.

TABLE 9

| SEQ ID NO. | Description | 0025-TCR12 |
|---|---|---|
| 97 | CDR1α | TISGNEY |
| 98 | CDR2α | GLKNN |
| 99 | CDR3α | IVRVGTSNSGYALN |
| 100 | Vα without signal | KTTQPTSMDCAEGRAANLPCNHSTISGNEYVYWYRQIH |
| | peptide (SignalP) | SQGPQYIIHGLKNNETNEMASLIITEDRKSSTLILPHA |
| | | TLRDTAVYYCIVRVGTSNSGYALNFGKGTSLLVTP |
| 101 | Vα only (without | MRLVARVTVFLTFGTIIDAKTTQPTSMDCAEGRAANLP |
| | the Constant) | CNHSTISGNEYVYWYRQIHSQGPQYIIHGLKNNETNEM |
| | | ASLIITEDRKSSTLILPHATLRDTAVYYCIVRVGTSNS |
| | | GYALNFGKGTSLLVTP |
| 102 | α chain with WT | MRLVARVTVFLTFGTIIDAKTTQPTSMDCAEGRAANLP |
| | signal peptide and | CNHSTISGNEYVYWYRQIHSQGPQYIIHGLKNNETNEM |
| | constant Cα | ASLIITEDRKSSTLILPHATLRDTAVYYCIVRVGTSNS |
| | | GYALNFGKGTSLLVTPNIQNPEPAVYQLKDPRSQDSTL |
| | | CLFTDFDSQINVPKTMESGTFITDKTVLDMKAMDSKSN |
| | | GAIAWSNQTSFTCQDIFKETNATYPSSDVPCDATLTEK |
| | | SFETDMNLNFQNLLVIVLRILLLKVAGFNLLMTLRLWS |
| | | S |
| 103 | CDR1β | SGHTA |
| 104 | CDR2β | FQGNSA |
| 105 | CDR3β | ASSWVVGLSTDTQY |
| 106 | Vβ without signal | GVSQSPSNKVTEKGKDVELRCDPISGHTALYWYRQSLG |
| | peptide (SignalP) | QGLEFLIYFQGNSAPDKSGLPSDRFSAERTGGSVSTLT |
| | | IQRTQQEDSAVYLCASSWVVGLSTDTQYFGPGTRLTVL |
| 107 | Vβ (without the | MGTRLLFWVAFCLLGADHTGAGVSQSPSNKVTEKGKDV |
| | Constant) | ELRCDPISGHTALYWYRQSLGQGLEFLIYFQGNSAPDK |
| | | SGLPSDRFSAERTGGSVSTLTIQRTQQEDSAVYLCASS |
| | | WVVGLSTDTQYFGPGTRLTVL |
| 108 | β chain with WT | MGTRLLFWVAFCLLGADHTGAGVSQSPSNKVTEKGKDV |
| | signal peptide and | ELRCDPISGHTALYWYRQSLGQGLEFLIYFQGNSAPDK |
| | constant Cβ | SGLPSDRFSAERTGGSVSTLTIQRTQQEDSAVYLCASS |
| | | WVVGLSTDTQYFGPGTRLTVLEDLRNVTPPKVSLFEPS |
| | | KAEIANKQKATLVCLARGFFPDHVELSWWVNGKEVHSG |
| | | VSTDPQAYKESNYSYCLSSRLRVSATFWHNPRNHFRCQ |
| | | VQFHGLSEEDKWPEGSPKPVTQNISAEAWGRADCGITS |
| | | ASYQQGVLSATILYEILLGKATLYAVLVSTLVVMAMVK |
| | | RKNS |

In some embodiments, 0025-TCR12 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR12 interacts with and/or is specific for the neoantigen in the context of HLA-DRA and DRB1*01:01.

TABLE 10

| SEQ ID NO. | Description | 0025-TCR30 |
|---|---|---|
| 109 | CDR1α | TTLSN |
| 110 | CDR2α | LVKSGEV |
| 111 | CDR3α | AGRGGGFKTI |
| 112 | Vα without signal | QQVMQIPQYQHVQEGEDFTTYCNSSTTLSNIQWYKQRPG |
| | peptide (SignalP) | GHPVFLIQLVKSGEVKKQKRLTFQFGEAKKNSSLHITAT |
| | | QTTDVGTYFCAGRGGGFKTIFGAGTRLFVKA |
| 113 | Vα only (without | MLLITSMLVLWMQLSQVNGQQVMQIPQYQHVQEGEDETT |
| | the Constant) | YCNSSTTLSNIQWYKQRPGGHPVFLIQLVKSGEVKKQKR |
| | | LTFQFGEAKKNSSLHITATQTTDVGTYFCAGRGGGFKTI |
| | | FGAGTRLFVKA |
| 114 | α chain with WT | MLLITSMLVLWMQLSQVNGQQVMQIPQYQHVQEGEDFTT |
| | signal peptide and | YCNSSTTLSNIQWYKQRPGGHPVFLIQLVKSGEVKKQKR |
| | constant Cα | LTFQFGEAKKNSSLHITATQTTDVGTYFCAGRGGGFKTI |
| | | FGAGTRLFVKANIQNPEPAVYQLKDPRSQDSTLCLFTDF |
| | | DSQINVPKTMESGTFITDKTVLDMKAMDSKSNGAIAWSN |
| | | QTSFTCQDIFKETNATYPSSDVPCDATLTEKSFETDMNL |
| | | NFQNLLVIVLRILLLKVAGFNLLMTLRLWSS |
| 115 | CDR1β | SGHTA |
| 116 | CDR2β | FQGNSA |
| 117 | CDR3β | ASSLRVMAGSNTGELF |
| 118 | Vβ without signal | GVSQSPSNKVTEKGKDVELRCDPISGHTALYWYRQSLGQ |
| | peptide (SignalP) | GLEFLIYFQGNSAPDKSGLPSDRFSAERTGGSVSTLTIQ |
| | | RTQQEDSAVYLCASSLRVMAGSNTGELFFGEGSRLTVL |
| 119 | Vβ (without the | MGTRLLFWVAFCLLGADHTGAGVSQSPSNKVTEKGKDVE |
| | Constant) | LRCDPISGHTALYWYRQSLGQGLEFLIYFQGNSAPDKSG |
| | | LPSDRFSAERTGGSVSTLTIQRTQQEDSAVYLCASSLRV |
| | | MAGSNTGELFFGEGSRLTVL |
| 120 | β chain with WT | MGTRLLFWVAFCLLGADHTGAGVSQSPSNKVTEKGKDVE |
| | signal peptide and | LRCDPISGHTALYWYRQSLGQGLEFLIYFQGNSAPDKSG |
| | constant Cβ | LPSDRFSAERTGGSVSTLTIQRTQQEDSAVYLCASSLRV |
| | | MAGSNTGELFFGEGSRLTVLEDLRNVTPPKVSLFEPSKA |
| | | EIANKQKATLVCLARGFFPDHVELSWWVNGKEVHSGVST |
| | | DPQAYKESNYSYCLSSRLRVSATFWHNPRNHFRCQVQFH |
| | | GLSEEDKWPEGSPKPVTQNISAEAWGRADCGITSASYQQ |
| | | GVLSATILYEILLGKATLYAVLVSTLVVMAMVKRKNS |

In some embodiments, 0025-TCR30 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR30 interacts with and/or is specific for the neoantigen in the context of HLA-DRA and DRB1*01:01.

TABLE 11

| SEQ ID NO. | Description | 0025-TCR31 |
|---|---|---|
| 121 | CDR1α | DSVNN |
| 122 | CDR2α | IPSGT |
| 123 | CDR3α | AVSMDSSYKLI |
| 124 | Vα without signal | IQVEQSPPDLILQEGANSTLRCNFSDSVNNLQWFHQNPW |
| | peptide (SignalP) | GQLINLFYIPSGTKQNGRLSATTVATERYSLLYISSSQT |
| | | TDSGVYFCAVSMDSSYKLIFGSGTRLLVRP |
| 125 | Vα only (without | MKRILGALLGLLSAQVCCVRGIQVEQSPPDLILQEGANS |
| | the Constant) | TLRCNFSDSVNNLQWFHQNPWGQLINLFYIPSGTKQNGR |
| | | LSATTVATERYSLLYISSSQTTDSGVYFCAVSMDSSYKL |
| | | IFGSGTRLLVRP |
| 126 | α chain with WT | MKRILGALLGLLSAQVCCVRGIQVEQSPPDLILQEGANS |
| | signal peptide and | TLRCNFSDSVNNLQWFHQNPWGQLINLFYIPSGTKQNGR |
| | constant Cα | LSATTVATERYSLLYISSSQTTDSGVYFCAVSMDSSYKL |
| | | IFGSGTRLLVRPNIQNPEPAVYQLKDPRSQDSTLCLFTD |
| | | FDSQINVPKTMESGTFITDKTVLDMKAMDSKSNGAIAWS |
| | | NQTSFTCQDIFKETNATYPSSDVPCDATLTEKSFETDMN |
| | | LNFQNLLVIVLRILLLKVAGFNLLMTLRLWSS |
| 127 | CDR1β | LGHDT |
| 128 | CDR2β | YNNKEL |
| 129 | CDR3β | ASNDRGRRTEAF |
| 130 | Vβ without signal | QTPKYLVTQMGNDKSIKCEQNLGHDTMYWYKQDSKKFLK |
| | peptide (SignalP) | IMFSYNNKELIINETVPNRFSPKSPDKAHLNLHINSLEL |
| | | GDSAVYFCASNDRGRRTEAFFGQGTRLTVV |
| 131 | Vβ (without the | MGCRLLCCVVFCLLQAGPLDTAVSQTPKYLVTQMGNDKS |
| | Constant) | IKCEQNLGHDTMYWYKQDSKKFLKIMFSYNNKELIINET |
| | | VPNRFSPKSPDKAHLNLHINSLELGDSAVYFCASNDRGR |
| | | RTEAFFGQGTRLTVV |
| 132 | β chain with WT | MGCRLLCCVVFCLLQAGPLDTAVSQTPKYLVTQMGNDKS |
| | signal peptide and | IKCEQNLGHDTMYWYKQDSKKFLKIMESYNNKELIINET |
| | constant Cβ | VPNRFSPKSPDKAHLNLHINSLELGDSAVYFCASNDRGR |
| | | RTEAFFGQGTRLTVVEDLRNVTPPKVSLFEPSKAEIANK |
| | | QKATLVCLARGFFPDHVELSWWVNGKEVHSGVSTDPQAY |
| | | KESNYSYCLSSRLRVSATFWHNPRNHFRCQVQFHGLSEE |
| | | DKWPEGSPKPVTQNISAEAWGRADCGITSASYQQGVLSA |
| | | TILYEILLGKATLYAVLVSTLVVMAMVKRKNS |

In some embodiments, 0025-TCR31 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR31 interacts with and/or is specific for the neoantigen in the context of HLA-DRA and DRB1*01:01.

TABLE 12

| SEQ ID NO. | Description | 0025-TCR32-1 |
|---|---|---|
| 133 | CDR1α | SSVSVY |
| 134 | CDR2α | YLSGSTLV |
| 135 | CDR3α | AVQFSSGGGADGLT |
| 136 | Vα without signal | QSVTQLDSQVPVFEEAPVELRCNYSSSVSVYLFWYVQYP |
| | peptide (SignalP) | NQGLQLLLKYLSGSTLVKGINGFEAEFNKSQTSFHLRKP |
| | | SVHISDTAEYFCAVQFSSGGGADGLTFGKGTHLIIQP |
| 137 | Vα only (without | MLLLLVPAFQVIFTLGGTRAQSVTQLDSQVPVFEEAPVE |
| | the Constant) | LRCNYSSSVSVYLFWYVQYPNQGLQLLLKYLSGSTLVKG |
| | | INGFEAEENKSQTSFHLRKPSVHISDTAEYFCAVQFSSG |
| | | GGADGLTFGKGTHLIIQP |
| 138 | α chain with WT | MLLLLVPAFQVIFTLGGTRAQSVTQLDSQVPVFEEAPVE |
| | signal peptide and | LRCNYSSSVSVYLFWYVQYPNQGLQLLLKYLSGSTLVKG |
| | constant Cα | INGFEAEFNKSQTSFHLRKPSVHISDTAEYFCAVQFSSG |
| | | GGADGLTFGKGTHLIIQPNIQNPEPAVYQLKDPRSQDST |
| | | LCLFTDFDSQINVPKTMESGTFITDKTVLDMKAMDSKSN |
| | | GAIAWSNQTSFTCQDIFKETNATYPSSDVPCDATLTEKS |
| | | FETDMNLNFQNLLVIVLRILLLKVAGFNLLMTLRLWSS |
| 139 | CDR1β | SGHDY |
| 140 | CDR2β | FNNNVP |
| 141 | CDR3β | ASSQNGLATDTQY |
| 142 | Vβ without signal | GVIQSPRHEVTEMGQEVTLRCKPISGHDYLFWYRQTMMR |
| | peptide (SignalP) | GLELLIYFNNNVPIDDSGMPEDRESAKMPNASESTLKIQ |
| | | PSEPRDSAVYFCASSQNGLATDTQYFGPGTRLTVL |
| 143 | Vβ (without the | MGSWTLCCVSLCILVAKHTDAGVIQSPRHEVTEMGQEVT |
| | Constant) | LRCKPISGHDYLFWYRQTMMRGLELLIYFNNNVPIDDSG |
| | | MPEDRFSAKMPNASFSTLKIQPSEPRDSAVYFCASSQNG |
| | | LATDTQYFGPGTRLTVL |
| 144 | β chain with WT | MGSWTLCCVSLCILVAKHTDAGVIQSPRHEVTEMGQEVT |
| | signal peptide and | LRCKPISGHDYLFWYRQTMMRGLELLIYFNNNVPIDDSG |
| | constant Cβ | MPEDRESAKMPNASFSTLKIQPSEPRDSAVYFCASSQNG |
| | | LATDTQYFGPGTRLTVLEDLRNVTPPKVSLFEPSKAEIA |
| | | NKQKATLVCLARGFFPDHVELSWWVNGKEVHSGVSTDPQ |
| | | AYKESNYSYCLSSRLRVSATFWHNPRNHFRCQVQFHGLS |
| | | EEDKWPEGSPKPVTQNISAEAWGRADCGITSASYQQGVL |
| | | SATILYEILLGKATLYAVLVSTLVVMAMVKRKNS |

In some embodiments, 0025-TCR32-1 interacts with and/or is specific for a peptide from gene GFRA2. In some embodiments, the peptide is from a neoantigen of GFRA2 and has the amino acid change R246H (in which position 246 of the GFRA2 protein is mutated from Arg to His). In some embodiments, 0025-TCR32-1 interacts with and/or is specific for the neoantigen in the context of HLA-DRA and DRB1*01:01.

TABLE 13

| SEQ ID NO. | Description | 0025-TCR33-1 |
|---|---|---|
| 145 | CDR1α | TSGFYG |
| 146 | CDR2α | NALDGL |
| 147 | CDR3α | AVVSGGYNKLI |
| 148 | Vα without signal | QSLEQPSEVTAVEGAIVQINCTYQTSGFYGLSWYQQHDGG |
| | peptide (SignalP) | APTFLSYNALDGLEETGRFSSFLSRSDSYGYLLLQELQMK |
| | | DSASYFCAVVSGGYNKLIFGAGTRLAVHP |
| 149 | Vα only (without | MWGAFLLYVSMKMGGTAGQSLEQPSEVTAVEGAIVQINCT |
| | the Constant) | YQTSGFYGLSWYQQHDGGAPTFLSYNALDGLEETGRFSSF |
| | | LSRSDSYGYLLLQELQMKDSASYFCAVVSGGYNKLIFGAG |
| | | TRLAVHP |
| 150 | α chain with WT | MWGAFLLYVSMKMGGTAGQSLEQPSEVTAVEGAIVQINCT |
| | signal peptide and | YQTSGFYGLSWYQQHDGGAPTFLSYNALDGLEETGRFSSF |
| | constant Cα | LSRSDSYGYLLLQELQMKDSASYFCAVVSGGYNKLIFGAG |
| | | TRLAVHPNIQNPEPAVYQLKDPRSQDSTLCLFTDFDSQIN |
| | | VPKTMESGTFITDKTVLDMKAMDSKSNGAIAWSNQTSFTC |
| | | QDIFKETNATYPSSDVPCDATLTEKSFETDMNLNFQNLLV |
| | | IVLRILLLKVAGFNLLMTLRLWSS |
| 151 | CDR1β | LNHDA |
| 152 | CDR2β | SQIVND |
| 153 | CDR3β | ASRLDSGANVLT |
| 154 | Vβ without signal | GITQSPKYLFRKEGQNVTLSCEQNLNHDAMYWYRQDPGQG |
| | peptide (SignalP) | LRLIYYSQIVNDFQKGDIAEGYSVSREKKESFPLTVTSAQ |
| | | KNPTAFYLCASRLDSGANVLTFGAGSRLTVL |
| 155 | Vβ (without the | MSNQVLCCVVLCLLGANTVDGGITQSPKYLFRKEGQNVTL |
| | Constant) | SCEQNLNHDAMYWYRQDPGQGLRLIYYSQIVNDFQKGDIA |
| | | EGYSVSREKKESFPLTVISAQKNPTAFYLCASRLDSGANV |
| | | LTFGAGSRLTVL |
| 156 | β chain with WT | MSNQVLCCVVLCLLGANTVDGGITQSPKYLFRKEGQNVTL |
| | signal peptide and | SCEQNLNHDAMYWYRQDPGQGLRLIYYSQIVNDFQKGDIA |
| | constant Cβ | EGYSVSREKKESFPLTVTSAQKNPTAFYLCASRLDSGANV |
| | | LTFGAGSRLTVLEDLRNVTPPKVSLFEPSKAEIANKQKAT |
| | | LVCLARGFFPDHVELSWWVNGKEVHSGVSTDPQAYKESNY |
| | | SYCLSSRLRVSATFWHNPRNHFRCQVQFHGLSEEDKWPEG |
| | | SPKPVTQNISAEAWGRADCGITSASYQQGVLSATILYEIL |
| | | LGKATLYAVLVSTLVVMAMVKRKNS |

In some embodiments, 0025-TCR33-1 interacts with and/or is specific for a peptide from gene GFRA2. In some embodiments, the peptide is from a neoantigen of GFRA2 and has the amino acid change R246H (in which position 246 of the GFRA2 protein is mutated from Arg to His). In some embodiments, 0025-TCR33-1 interacts with and/or is specific for the neoantigen in the context of HLA-DRA and DRB1*01:01.

TABLE 14

| SEQ ID NO. | Description | 0025-TCR36 |
|---|---|---|
| 157 | CDR1α | TRDTTYY |
| 158 | CDR2α | RNSFDEQN |
| 159 | CDR3α | ALSEERPGTASKLT |
| 160 | Vα without signal | QKVTQAQTEISVVEKEDVTLDCVYETRDTTYYLFWYKQPP |
| | peptide (SignalP) | SGELVFLIRRNSFDEQNEISGRYSWNFQKSTSSFNFTITA |
| | | SQVVDSAVYFCALSEERPGTASKLTFGTGTRLQVTL |
| 161 | Vα only (without | MLTASLLRAVIASICVVSSMAQKVTQAQTEISVVEKEDVT |
| | the Constant) | LDCVYETRDTTYYLFWYKQPPSGELVFLIRRNSFDEQNEI |
| | | SGRYSWNFQKSTSSFNFTITASQVVDSAVYFCALSEERPG |
| | | TASKLTFGTGTRLQVTL |
| 162 | α chain with WT | MLTASLLRAVIASICVVSSMAQKVTQAQTEISVVEKEDVT |
| | signal peptide and | LDCVYETRDTTYYLFWYKQPPSGELVFLIRRNSFDEQNEI |
| | constant Cα | SGRYSWNFQKSTSSFNFTITASQVVDSAVYFCALSEERPG |
| | | TASKLTFGTGTRLQVTLNIQNPEPAVYQLKDPRSQDSTLC |
| | | LFTDFDSQINVPKTMESGTFITDKTVLDMKAMDSKSNGAI |
| | | AWSNQTSFTCQDIFKETNATYPSSDVPCDATLTEKSFETD |
| | | MNLNFQNLLVIVLRILLLKVAGFNLLMTLRLWSS |
| 163 | CDR1β | SGHTA |
| 164 | CDR2β | FQGTGA |
| 165 | CDR3β | ASSLTGTVTGTDTQY |
| 166 | Vβ without signal | GVSQTPSNKVTEKGKYVELRCDPISGHTALYWYRQSLGQG |
| | peptide (SignalP) | PEFLIYFQGTGAADDSGLPNDRFFAVRPEGSVSTLKIQRT |
| | | ERGDSAVYLCASSLTGTVTGTDTQYFGPGTRLTVL |
| 167 | Vβ (without the | MGTRLLCWAALCLLGADHTGAGVSQTPSNKVTEKGKYVEL |
| | Constant) | RCDPISGHTALYWYRQSLGQGPEFLIYFQGTGAADDSGLP |
| | | NDRFFAVRPEGSVSTLKIQRTERGDSAVYLCASSLTGTVT |
| | | GTDTQYFGPGTRLTVL |
| 168 | β chain with WT | MGTRLLCWAALCLLGADHTGAGVSQTPSNKVTEKGKYVEL |
| | signal peptide and | RCDPISGHTALYWYRQSLGQGPEFLIYFQGTGAADDSGLP |
| | constant Cβ | NDRFFAVRPEGSVSTLKIQRTERGDSAVYLCASSLTGTVT |
| | | GTDTQYFGPGTRLTVLEDLRNVTPPKVSLFEPSKAEIANK |
| | | QKATLVCLARGFFPDHVELSWWVNGKEVHSGVSTDPQAYK |
| | | ESNYSYCLSSRLRVSATFWHNPRNHFRCQVQFHGLSEEDK |
| | | WPEGSPKPVTQNISAEAWGRADCGITSASYQQGVLSATIL |
| | | YEILLGKATLYAVLVSTLVVMAMVKRKNS |

In some embodiments, 0025-TCR36 interacts with and/or is specific for a peptide from gene GFRA2. In some embodiments, the peptide is from a neoantigen of GFRA2 and has the amino acid change R246H (in which position 246 of the GFRA2 protein is mutated from Arg to His). In some embodiments, 0025-TCR36 interacts with and/or is specific for the neoantigen in the context of HLA-DRA and DRB1*01:01.

TABLE 15

| SEQ ID NO. | Description | 0025-TCR43-1 |
|---|---|---|
| 169 | CDR1α | ATGYPS |
| 170 | CDR2α | ATKADDK |
| 171 | CDR3α | ALRTGANSKLT |
| 172 | Vα without signal | NSVTQMEGPVTLSEEAFLTINCTYTATGYPSLFWYVQYPG |
| | peptide (SignalP) | EGLQLLLKATKADDKGSNKGFEATYRKETTSFHLEKGSVQ |
| | | VSDSAVYFCALRTGANSKLTFGKGITLSVRP |
| 173 | Vα ony (without | MNYSPGLVSLILLLLGRTRGNSVTQMEGPVTLSEEAFLTI |
| | the Constant) | NCTYTATGYPSLFWYVQYPGEGLQLLLKATKADDKGSNKG |
| | | FEATYRKETTSFHLEKGSVQVSDSAVYFCALRTGANSKLT |
| | | FGKGITLSVRP |
| 174 | α chain with WT | MNYSPGLVSLILLLLGRTRGNSVTQMEGPVTLSEEAFLTI |
| | signal peptide and | NCTYTATGYPSLFWYVQYPGEGLQLLLKATKADDKGSNKG |
| | constant Cα | FEATYRKETTSFHLEKGSVQVSDSAVYFCALRTGANSKLT |
| | | FGKGITLSVRPNIQNPEPAVYQLKDPRSQDSTLCLFTDED |
| | | SQINVPKTMESGTFITDKTVLDMKAMDSKSNGAIAWSNQT |
| | | SFTCQDIFKETNATYPSSDVPCDATLTEKSFETDMNLNFQ |
| | | NLLVIVLRILLLKVAGFNLLMTLRLWSS |
| 175 | CDR1β | SGHDN |
| 176 | CDR2β | FVKESK |
| 177 | CDR3β | ASSLSQSSNYGYT |
| 178 | Vβ without signal | GVTQFPSHSVIEKGQTVTLRCDPISGHDNLYWYRRVMGKE |
| | peptide (SignalP) | IKFLLHFVKESKQDESGMPNNRFLAERTGGTYSTLKVQPA |
| | | ELEDSGVYFCASSLSQSSNYGYTFGSGTRLTVV |
| 179 | Vβ (without the | MVSRLLSLVSLCLLGAKHIEAGVTQFPSHSVIEKGQTVTL |
| | Constant) | RCDPISGHDNLYWYRRVMGKEIKFLLHFVKESKQDESGMP |
| | | NNRFLAERTGGTYSTLKVQPAELEDSGVYFCASSLSQSSN |
| | | YGYTFGSGTRLTVV |
| 180 | β chain with WT | MVSRLLSLVSLCLLGAKHIEAGVTQFPSHSVIEKGQTVTL |
| | signal peptide and | RCDPISGHDNLYWYRRVMGKEIKFLLHFVKESKQDESGMP |
| | constant Cβ | NNRFLAERTGGTYSTLKVQPAELEDSGVYFCASSLSQSSN |
| | | YGYTFGSGTRLTVVEDLRNVTPPKVSLFEPSKAEIANKQK |
| | | ATLVCLARGFFPDHVELSWWVNGKEVHSGVSTDPQAYKES |
| | | NYSYCLSSRLRVSATFWHNPRNHFRCQVQFHGLSEEDKWP |
| | | EGSPKPVTQNISAEAWGRADCGITSASYQQGVLSATILYE |
| | | ILLGKATLYAVLVSTLVVMAMVKRKNS |

In some embodiments, 0025-TCR43-1 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR43-1 interacts with and/or is specific for the neoantigen in the context of HLA-DRA and DRB1*01:01.

TABLE 16

| SEQ ID NO. | Description | 0025-TCR45 |
|---|---|---|
| 181 | CDR1α | NSASQS |
| 182 | CDR2α | VYSSGN |
| 183 | CDR3α | VVNTRGGYNKLI |
| 184 | Vα without signal | QRKEVEQDPGPFNVPEGATVAFNCTYSNSASQSFFWYRQD |
| | peptide (SignalP) | CRKEPKLLMSVYSSGNEDGRETAQLNRASQYISLLIRDSK |
| | | LSDSATYLCVVNTRGGYNKLIFGAGTRLAVHP |
| 185 | Vα only (without | MISLRVLLVILWLQLSWVWSQRKEVEQDPGPENVPEGATV |
| | the Constant) | AFNCTYSNSASQSFFWYRQDCRKEPKLLMSVYSSGNEDGR |
| | | FTAQLNRASQYISLLIRDSKLSDSATYLCVVNTRGGYNKL |
| | | IFGAGTRLAVHP |
| 186 | α chain with WT | MISLRVLLVILWLQLSWVWSQRKEVEQDPGPFNVPEGATV |
| | signal peptide and | AFNCTYSNSASQSFFWYRQDCRKEPKLLMSVYSSGNEDGR |
| | constant Cα | FTAQLNRASQYISLLIRDSKLSDSATYLCVVNTRGGYNKL |
| | | IFGAGTRLAVHPNIQNPEPAVYQLKDPRSQDSTLCLFTDE |
| | | DSQINVPKTMESGTFITDKTVLDMKAMDSKSNGAIAWSNQ |
| | | TSFTCQDIFKETNATYPSSDVPCDATLTEKSFETDMNLNF |
| | | QNLLVIVLRILLLKVAGFNLLMTLRLWSS |
| 187 | CDR1β | SGHTA |
| 188 | CDR2β | FQGNSA |
| 189 | CDR3β | ASSLAVGGTEAF |
| 190 | Vβ without signal | VSQSPSNKVTEKGKDVELRCDPISGHTALYWYRQRLGQGL |
| | peptide (SignalP) | EFLIYFQGNSAPDKSGLPSDRFSAERTGESVSTLTIQRTQ |
| | | QEDSAVYLCASSLAVGGTEAFFGQGTRLTVV |
| 191 | Vβ (without the | MGTRLLFWVAFCLLGAYHTGAGVSQSPSNKVTEKGKDVEL |
| | Constant) | RCDPISGHTALYWYRQRLGQGLEFLIYFQGNSAPDKSGLP |
| | | SDRFSAERTGESVSTLTIQRTQQEDSAVYLCASSLAVGGT |
| | | EAFFGQGTRLTVV |
| 192 | β chain with WT | MGTRLLFWVAFCLLGAYHTGAGVSQSPSNKVTEKGKDVEL |
| | signal peptide and | RCDPISGHTALYWYRQRLGQGLEFLIYFQGNSAPDKSGLP |
| | constant Cβ | SDRFSAERTGESVSTLTIQRTQQEDSAVYLCASSLAVGGT |
| | | EAFFGQGTRLTVVEDLRNVTPPKVSLFEPSKAEIANKQKA |
| | | TLVCLARGFFPDHVELSWWVNGKEVHSGVSTDPQAYKESN |
| | | YSYCLSSRLRVSATFWHNPRNHFRCQVQFHGLSEEDKWPE |
| | | GSPKPVTQNISAEAWGRADCGITSASYQQGVLSATILYEI |
| | | LLGKATLYAVLVSTLVVMAMVKRKNS |

In some embodiments, 0025-TCR45 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR45 interacts with and/or is specific for the neoantigen in the context of HLA-DRA and DRB1*01:01.

TABLE 17

| SEQ ID NO. | Description | 0025-TCR47 |
|---|---|---|
| 193 | CDR1α | SIFNT |
| 194 | CDR2α | LYKAGEL |
| 195 | CDR3α | AGPGGATNKLI |
| 196 | Vα without signal | QQLNQSPQSMFIQEGEDVSMNCTSSSIFNTWLWYKQDPG |
| | peptide (SignalP) | EGPVLLIALYKAGELTSNGRLTAQFGITRKDSFLNISAS |
| | | IPSDVGIYFCAGPGGATNKLIFGTGTLLAVQP |
| 197 | Vα only (without | MLLEHLLIILWMQLTWVSGQQLNQSPQSMFIQEGEDVSM |
| | the Constant) | NCTSSSIFNTWLWYKQDPGEGPVLLIALYKAGELTSNGR |
| | | LTAQFGITRKDSFLNISASIPSDVGIYFCAGPGGATNKL |
| | | IFGTGTLLAVQP |
| 198 | α chain with WT | MLLEHLLIILWMQLTWVSGQQLNQSPQSMFIQEGEDVSM |
| | signal peptide and | NCTSSSIFNTWLWYKQDPGEGPVLLIALYKAGELTSNGR |
| | constant Cα | LTAQFGITRKDSFLNISASIPSDVGIYFCAGPGGATNKL |
| | | IFGTGTLLAVQPNIQNPEPAVYQLKDPRSQDSTLCLFTD |
| | | FDSQINVPKTMESGTFITDKTVLDMKAMDSKSNGAIAWS |
| | | NQTSFTCQDIFKETNATYPSSDVPCDATLTEKSFETDMN |
| | | LNFQNLLVIVLRILLLKVAGFNLLMTLRLWSS |
| 199 | CDR1β | MNHNY |
| 200 | CDR2β | SVGAGI |
| 201 | CDR3β | ASRRSTSGLQETQY |
| 202 | Vβ without signal | GVTQTPKFRILKIGQSMTLQCTQDMNHNYMYWYRQDPGM |
| | peptide (SignalP) | GLKLIYYSVGAGITDKGEVPNGYNVSRSTTEDFPLRLEL |
| | | AAPSQTSVYFCASRRSTSGLQETQYFGPGTRLLVL |
| 203 | Vβ (without the | MSISLLCCAAFPLLWAGPVNAGVTQTPKFRILKIGQSMT |
| | Constant) | LQCTQDMNHNYMYWYRQDPGMGLKLIYYSVGAGITDKGE |
| | | VPNGYNVSRSTTEDFPLRLELAAPSQTSVYFCASRRSTS |
| | | GLQETQYFGPGTRLLVL |
| 204 | β chain with WT | MSISLLCCAAFPLLWAGPVNAGVTQTPKFRILKIGQSMT |
| | signal peptide and | LQCTQDMNHNYMYWYRQDPGMGLKLIYYSVGAGITDKGE |
| | constant Cβ | VPNGYNVSRSTTEDEPLRLELAAPSQTSVYFCASRRSTS |
| | | GLQETQYFGPGTRLLVLEDLRNVTPPKVSLFEPSKAEIA |
| | | NKQKATLVCLARGFFPDHVELSWWVNGKEVHSGVSTDPQ |
| | | AYKESNYSYCLSSRLRVSATFWHNPRNHFRCQVQFHGLS |
| | | EEDKWPEGSPKPVTQNISAEAWGRADCGITSASYQQGVL |
| | | SATILYEILLGKATLYAVLVSTLVVMAMVKRKNS |

In some embodiments, 0025-TCR47 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR47 interacts with and/or is specific for the 5 neoantigen in the context of HLA-DRA and DRB1*01:01.

TABLE 18

| SEQ ID NO. | Description | 0025-TCR48 |
|---|---|---|
| 205 | CDR1α | VSGNPY |
| 206 | CDR2α | YITGDNLV |
| 207 | CDR3α | AVRDNTGGFKTI |
| 208 | Vα without signal | QSVAQPEDQVNVAEGNPLTVKCTYSVSGNPYLFWYVQYPN |
| | peptide (SignalP) | RGLQFLLKYITGDNLVKGSYGFEAEFNKSQTSFHLKKPSA |
| | | LVSDSALYFCAVRDNTGGFKTIFGAGTRLFVKA |
| 209 | Vα only (without | MASAPISMLAMLFTLSGLRAQSVAQPEDQVNVAEGNPLTV |
| | the Constant) | KCTYSVSGNPYLFWYVQYPNRGLQFLLKYITGDNLVKGSY |
| | | GFEAEFNKSQTSFHLKKPSALVSDSALYFCAVRDNTGGFK |
| | | TIFGAGTRLFVKA |
| 210 | α chain with WT | MASAPISMLAMLFTLSGLRAQSVAQPEDQVNVAEGNPLTV |
| | signal peptide and | KCTYSVSGNPYLFWYVQYPNRGLQFLLKYITGDNLVKGSY |
| | constant Cα | GFEAEFNKSQTSFHLKKPSALVSDSALYFCAVRDNTGGFK |
| | | TIFGAGTRLFVKANIQNPEPAVYQLKDPRSQDSTLCLFTD |
| | | FDSQINVPKTMESGTFITDKTVLDMKAMDSKSNGAIAWSN |
| | | QTSFTCQDIFKETNATYPSSDVPCDATLTEKSFETDMNLN |
| | | FQNLLVIVLRILLLKVAGFNLLMTLRLWSS |
| 211 | CDR1β | SGHAT |
| 212 | CDR2β | FQNNGV |
| 213 | CDR3β | ASSRRTSGSSYNEQF |
| 214 | Vβ without signal | GVAQSPRYKIIEKRQSVAFWCNPISGHATLYWYQQILGQG |
| | peptide (SignalP) | PKLLIQFQNNGVVDDSQLPKDRFSAERLKGVDSTLKIQPA |
| | | KLEDSAVYLCASSRRTSGSSYNEQFFGPGTRLTVL |
| 215 | Vβ (without the | MGTRLLCWAALCLLGAELTEAGVAQSPRYKIIEKRQSVAF |
| | Constant) | WCNPISGHATLYWYQQILGQGPKLLIQFQNNGVVDDSQLP |
| | | KDRFSAERLKGVDSTLKIQPAKLEDSAVYLCASSRRTSGS |
| | | SYNEQFFGPGTRLTVL |
| 216 | β chain with WT | MGTRLLCWAALCLLGAELTEAGVAQSPRYKIIEKRQSVAF |
| | signal peptide and | WCNPISGHATLYWYQQILGQGPKLLIQFQNNGVVDDSQLP |
| | constant Cβ | KDRFSAERLKGVDSTLKIQPAKLEDSAVYLCASSRRTSGS |
| | | SYNEQFFGPGTRLTVLEDLRNVTPPKVSLFEPSKAEIANK |
| | | QKATLVCLARGFFPDHVELSWWVNGKEVHSGVSTDPQAYK |
| | | ESNYSYCLSSRLRVSATFWHNPRNHFRCQVQFHGLSEEDK |
| | | WPEGSPKPVTQNISAEAWGRADCGITSASYQQGVLSATIL |
| | | YEILLGKATLYAVLVSTLVVMAMVKRKNS |

In some embodiments, 0025-TCR48 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR48 interacts with and/or is specific for the neoantigen in the context of HLA-DRA and DRB1*01:01.

TABLE 19

| SEQ ID NO. | Description | 0025-TCR52 |
|---|---|---|
| 217 | CDR1α | VTNERS |
| 218 | CDR2α | LTSSGIE |
| 219 | CDR3α | ALRGSGAGSYQLT |
| 220 | Vα without signal | EDKVVQSPLSLVVHEGDTVTLNCSYEVTNERSLLWYKQEK |
| | peptide (SignalP) | KAPTFLFMLTSSGIEKKSGRLSSILDKKELFSILNITATQ |
| | | TGDSAVYLCALRGSGAGSYQLTFGKGTKLSVIP |
| 221 | Vα only (without | MMKCPQALLAIFWLLLSWVSSEDKVVQSPLSLVVHEGDTV |
| | the Constant) | TLNCSYEVTNERSLLWYKQEKKAPTFLEMLTSSGIEKKSG |
| | | RLSSILDKKELFSILNITATQTGDSAVYLCALRGSGAGSY |
| | | QLTFGKGTKLSVIP |
| 222 | α chain with WT | MMKCPQALLAIFWLLLSWVSSEDKVVQSPLSLVVHEGDTV |
| | signal peptide and | TLNCSYEVINFRSLLWYKQEKKAPTFLEMLTSSGIEKKSG |
| | constant Cα | RLSSILDKKELFSILNITATQTGDSAVYLCALRGSGAGSY |
| | | QLTFGKGTKLSVIPNIQNPEPAVYQLKDPRSQDSTLCLFT |
| | | DFDSQINVPKTMESGTFITDKTVLDMKAMDSKSNGAIAWS |
| | | NQTSFTCQDIFKETNATYPSSDVPCDATLTEKSFETDMNL |
| | | NFQNLLVIVLRILLLKVAGFNLLMTLRLWSS |
| 223 | CDR1β | SGHVS |
| 224 | CDR2β | FQNEAQ |
| 225 | CDR3β | ASSLEGGGPNEQF |
| 226 | Vβ without signal | GVSQSPRYKVAKRGQDVALRCDPISGHVSLFWYQQALGQG |
| | peptide (SignalP) | PEFLTYFQNEAQLDKSGLPSDRFFAERPEGSVSTLKIQRT |
| | | QQEDSAVYLCASSLEGGGPNEQFFGPGTRLTVL |
| 227 | Vβ (without the | MGTRLLCWVVLGFLGTDHTGAGVSQSPRYKVAKRGQDVAL |
| | Constant) | RCDPISGHVSLFWYQQALGQGPEFLTYFQNEAQLDKSGLP |
| | | SDRFFAERPEGSVSTLKIQRTQQEDSAVYLCASSLEGGGP |
| | | NEQFFGPGTRLTVL |
| 228 | β chain with WT | MGTRLLCWVVLGFLGTDHTGAGVSQSPRYKVAKRGQDVAL |
| | signal peptide and | RCDPISGHVSLFWYQQALGQGPEFLTYFQNEAQLDKSGLP |
| | constant Cβ | SDRFFAERPEGSVSTLKIQRTQQEDSAVYLCASSLEGGGP |
| | | NEQFFGPGTRLTVLEDLRNVTPPKVSLFEPSKAEIANKQK |
| | | ATLVCLARGFFPDHVELSWWVNGKEVHSGVSTDPQAYKES |
| | | NYSYCLSSRLRVSATFWHNPRNHFRCQVQFHGLSEEDKWP |
| | | EGSPKPVTQNISAEAWGRADCGITSASYQQGVLSATILYE |
| | | ILLGKATLYAVLVSTLVVMAMVKRKNS |

In some embodiments, 0025-TCR52 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR52 interacts with and/or is specific for the neoantigen in the context of HLA-DRA and DRB1*01:01.

TABLE 20

| SEQ ID NO. | Description | 0025-TCR62 |
|---|---|---|
| 229 | CDR1α | ATGYPS |
| 230 | CDR2α | ATKADDK |
| 231 | CDR3α | ALSTGSARQLT |
| 232 | Vα without signal | NSVTQMEGPVTLSEEAFLTINCTYTATGYPSLFWYVQYPGEG |
| | peptide (SignalP) | LQLLLKATKADDKGSNKGFEATYRKETTSFHLEKGSVQVSDS |
| | | AVYFCALSTGSARQLTFGSGTQLTVLP |
| 233 | Vα only (without | MNYSPGLVSLILLLLGRTRGNSVTQMEGPVTLSEEAFLTINC |
| | the Constant) | TYTATGYPSLFWYVQYPGEGLQLLLKATKADDKGSNKGFEAT |
| | | YRKETTSFHLEKGSVQVSDSAVYFCALSTGSARQLTFGSGTQ |
| | | LTVLP |
| 234 | α chain with WT | MNYSPGLVSLILLLLGRTRGNSVTQMEGPVTLSEEAFLTINC |
| | signal peptide and | TYTATGYPSLFWYVQYPGEGLQLLLKATKADDKGSNKGFEAT |
| | constant Cα | YRKETTSFHLEKGSVQVSDSAVYFCALSTGSARQLTFGSGTQ |
| | | LTVLPNIQNPEPAVYQLKDPRSQDSTLCLFTDFDSQINVPKT |
| | | MESGTFITDKTVLDMKAMDSKSNGAIAWSNQTSFTCQDIFKE |
| | | TNATYPSSDVPCDATLTEKSFETDMNLNFQNLLVIVLRILLL |
| | | KVAGFNLLMTLRLWSS |
| 235 | CDR1β | LNHDA |
| 236 | CDR2β | SQIVND |
| 237 | CDR3β | ASSISGTVSGANVLT |
| 238 | Vβ without signal | GITQSPKYLFRKEGQNVTLSCEQNLNHDAMYWYRQDPGQGLR |
| | peptide (SignalP) | LIYYSQIVNDFQKGDIAEGYSVSREKKESFPLTVTSAQKNPT |
| | | AFYLCASSISGTVSGANVLTFGAGSRLTVL |
| 239 | Vβ (without the | MSNQVLCCVVLCFLGANTVDGGITQSPKYLFRKEGQNVTLSC |
| | Constant) | EQNLNHDAMYWYRQDPGQGLRLIYYSQIVNDFQKGDIAEGYS |
| | | VSREKKESFPLTVTSAQKNPTAFYLCASSISGTVSGANVLTF |
| | | GAGSRLTVL |
| 240 | β chain with WT | MSNQVLCCVVLCFLGANTVDGGITQSPKYLFRKEGQNVTLSC |
| | signal peptide and | EQNLNHDAMYWYRQDPGQGLRLIYYSQIVNDFQKGDIAEGYS |
| | constant Cβ | VSREKKESFPLTVTSAQKNPTAFYLCASSISGTVSGANVLTF |
| | | GAGSRLTVLEDLRNVTPPKVSLEEPSKAEIANKQKATLVCLA |
| | | RGFFPDHVELSWWVNGKEVHSGVSTDPQAYKESNYSYCLSSR |
| | | LRVSATFWHNPRNHFRCQVQFHGLSEEDKWPEGSPKPVTQNI |
| | | SAEAWGRADCGITSASYQQGVLSATILYEILLGKATLYAVLV |
| | | STLVVMAMVKRKNS |

In some embodiments, 0025-TCR62 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR62 interacts with and/or is specific for the neoantigen in the context of HLA-DRA and DRB1*01:01.

TABLE 21

| SEQ ID NO. | Description | 0025-TCR69 |
|---|---|---|
| 241 | CDR1α | TSGFYG |
| 242 | CDR2α | NALDGL |
| 243 | CDR3α | AVLSGGYNKLI |
| 244 | Vα without signal | QSLEQPSEVTAVEGAIVQINCTYQTSGFYGLSWYQQHDGGAP |
| | peptide (SignalP) | TFLSYNALDGLEETGRFSSFLSRSDSYGYLLLQELQMKDSAS |
| | | YFCAVLSGGYNKLIFGAGTRLAVHP |
| 245 | Vα only (without | MWGAFLLYVSMKMGGTAGQSLEQPSEVTAVEGAIVQINCTYQ |
| | the Constant) | TSGFYGLSWYQQHDGGAPTFLSYNALDGLEETGRFSSFLSRS |
| | | DSYGYLLLQELQMKDSASYFCAVLSGGYNKLIFGAGTRLAVH |
| | | P |
| 246 | α chain with WT | MWGAFLLYVSMKMGGTAGQSLEQPSEVTAVEGAIVQINCTYQ |
| | signal peptide and | TSGFYGLSWYQQHDGGAPTFLSYNALDGLEETGRFSSFLSRS |
| | constant Cα | DSYGYLLLQELQMKDSASYFCAVLSGGYNKLIFGAGTRLAVH |
| | | PNIQNPEPAVYQLKDPRSQDSTLCLFTDFDSQINVPKTMESG |
| | | TFITDKTVLDMKAMDSKSNGAIAWSNQTSFTCQDIFKETNAT |
| | | YPSSDVPCDATLTEKSFETDMNLNFQNLLVIVLRILLLKVAG |
| | | FNLLMTLRLWSS |
| 247 | CDR1β | LNHDA |
| 248 | CDR2β | SQIVND |
| 249 | CDR3β | ASRKDSGENGYT |
| 250 | Vβ without signal | GITQSPKYLFRKEGQNVTLSCEQNLNHDAMYWYRQDPGQGLR |
| | peptide (SignalP) | LIYYSQIVNDFQKGDIAEGYSVSREKKESFPLTVTSAQKNPT |
| | | AFYLCASRKDSGENGYTFGSGTRLTVV |
| 251 | Vβ (without the | MSNQVLCCVVLCFLGANTVDGGITQSPKYLFRKEGQNVTLSC |
| | Constant) | EQNLNHDAMYWYRQDPGQGLRLIYYSQIVNDFQKGDIAEGYS |
| | | VSREKKESFPLTVTSAQKNPTAFYLCASRKDSGENGYTFGSG |
| | | TRLTVV |
| 252 | β chain with WT | MSNQVLCCVVLCFLGANTVDGGITQSPKYLFRKEGQNVTLSC |
| | signal peptide and | EQNLNHDAMYWYRQDPGQGLRLIYYSQIVNDFQKGDIAEGYS |
| | constant Cβ | VSREKKESFPLTVTSAQKNPTAFYLCASRKDSGENGYTFGSG |
| | | TRLTVVEDLRNVTPPKVSLFEPSKAEIANKQKATLVCLARGE |
| | | FPDHVELSWWVNGKEVHSGVSTDPQAYKESNYSYCLSSRLRV |
| | | SATFWHNPRNHFRCQVQFHGLSEEDKWPEGSPKPVTQNISAE |
| | | AWGRADCGITSASYQQGVLSATILYEILLGKATLYAVLVSTL |
| | | VVMAMVKRKNS |

In some embodiments, 0025-TCR69 interacts with and/or is specific for a peptide from gene GFRA2. In some embodiments, the peptide is from a neoantigen of GFRA2 and has the amino acid change R246H (in which position 246 of the GFRA2 protein is mutated from Arg to His). In some embodiments, 0025-TCR69 interacts with and/or is specific for the neoantigen in the context of HLA-DRA and DRB1*01:01.

TABLE 22

| SEQ ID NO. | Description | 0025-TCR72 |
|---|---|---|
| 253 | CDR1α | VGISA |
| 254 | CDR2α | LSSGK |
| 255 | CDR3α | AVWEETSGSRLT |
| 256 | Vα without signal | AKNEVEQSPQNLTAQEGEFITINCSYSVGISALHWLQQHPGG |
| | peptide (SignalP) | GIVSLFMLSSGKKKHGRLIATINIQEKHSSLHITASHPRDSA |
| | | VYICAVWEETSGSRLTFGEGTQLTVNP |
| 257 | Vα only (without | MVKIRQFLLAILWLQLSCVSAAKNEVEQSPQNLTAQEGEFIT |
| | the Constant) | INCSYSVGISALHWLQQHPGGGIVSLFMLSSGKKKHGRLIAT |
| | | INIQEKHSSLHITASHPRDSAVYICAVWEETSGSRLTFGEGT |
| | | QLTVNP |
| 258 | α chain with WT | MVKIRQFLLAILWLQLSCVSAAKNEVEQSPQNLTAQEGEFIT |
| | signal peptide and | INCSYSVGISALHWLQQHPGGGIVSLFMLSSGKKKHGRLIAT |
| | constant Cα | INIQEKHSSLHITASHPRDSAVYICAVWEETSGSRLTFGEGT |
| | | QLTVNPNIQNPEPAVYQLKDPRSQDSTLCLFTDFDSQINVPK |
| | | TMESGTFITDKTVLDMKAMDSKSNGAIAWSNQTSFTCQDIFK |
| | | ETNATYPSSDVPCDATLTEKSFETDMNLNFQNLLVIVLRILL |
| | | LKVAGFNLLMTLRLWSS |
| 259 | CDR1β | SNHLY |
| 260 | CDR2β | FYNNEI |
| 261 | CDR3β | ASTRDTWSTDTQY |
| 262 | Vβ without signal | EPEVTQTPSHQVTQMGQEVILCCVPISNHLYFYWYRQILGQK |
| | peptide (SignalP) | VEFLVSFYNNEISEKSEIFDDQFSVERPDGSNFTLKIRSTKL |
| | | EDSAMYFCASTRDTWSTDTQYFGPGTRLTVL |
| 263 | Vβ (without the | MDTWLVCWAIFSLLKAGLTEPEVTQTPSHQVTQMGQEVILCC |
| | Constant) | VPISNHLYFYWYRQILGQKVEFLVSFYNNEISEKSEIFDDQF |
| | | SVERPDGSNFTLKIRSTKLEDSAMYFCASTRDTWSTDTQYFG |
| | | PGTRLTVL |
| 264 | β chain with WT | MDTWLVCWAIFSLLKAGLTEPEVTQTPSHQVTQMGQEVILCC |
| | signal peptide and | VPISNHLYFYWYRQILGQKVEFLVSFYNNEISEKSEIFDDQF |
| | constant Cβ | SVERPDGSNFTLKIRSTKLEDSAMYFCASTRDTWSTDTQYFG |
| | | PGTRLTVLEDLRNVTPPKVSLFEPSKAEIANKQKATLVCLAR |
| | | GFFPDHVELSWWVNGKEVHSGVSTDPQAYKESNYSYCLSSRL |
| | | RVSATFWHNPRNHFRCQVQFHGLSEEDKWPEGSPKPVTQNIS |
| | | AEAWGRADCGITSASYQQGVLSATILYEILLGKATLYAVLVS |
| | | TLVVMAMVKRKNS |

In some embodiments, 0025-TCR72 interacts with and/or is specific for a peptide from gene GFRA2. In some embodiments, the peptide is from a neoantigen of GFRA2 and has the amino acid change R246H (in which position 246 of the GFRA2 protein is mutated from Arg to His). In some embodiments, 0025-TCR72 interacts with and/or is specific for the neoantigen in the context of HLA-DRA and DRB1*01:01.

TABLE 23

| SEQ ID NO. | Description | 0025-TCR77 |
|---|---|---|
| 265 | CDR1α | DRGSQS |
| 266 | CDR2α | IYSNGD |
| 267 | CDR3α | AVKASSGSARQLT |
| 268 | Vα without signal | QQKEVEQNSGPLSVPEGAIASLNCTYSDRGSQSFFWYRQYS |
| | peptide (SignalP) | GKSPELIMFIYSNGDKEDGRFTAQLNKASQYVSLLIRDSQP |
| | | SDSATYLCAVKASSGSARQLTFGSGTQLTVLP |
| 269 | Vα only (without | MKSLRVLLVILWLQLSWVWSQQKEVEQNSGPLSVPEGAIAS |
| | the Constant) | LNCTYSDRGSQSFFWYRQYSGKSPELIMFIYSNGDKEDGRF |
| | | TAQLNKASQYVSLLIRDSQPSDSATYLCAVKASSGSARQLT |
| | | FGSGTQLTVLP |
| 270 | α chain with WT | MKSLRVLLVILWLQLSWVWSQQKEVEQNSGPLSVPEGAIAS |
| | signal peptide and | LNCTYSDRGSQSFFWYRQYSGKSPELIMFIYSNGDKEDGRF |
| | constant Cα | TAQLNKASQYVSLLIRDSQPSDSATYLCAVKASSGSARQLT |
| | | FGSGTQLTVLPNIQNPEPAVYQLKDPRSQDSTLCLFTDEDS |
| | | QINVPKTMESGTFITDKTVLDMKAMDSKSNGAIAWSNQTSF |
| | | TCQDIFKETNATYPSSDVPCDATLTEKSFETDMNLNFQNLL |
| | | VIVLRILLLKVAGFNLLMTLRLWSS |
| 271 | CDR1β | MNHEY |
| 272 | CDR2β | SVGEGT |
| 273 | CDR3β | ASSYKLAGDNEQF |
| 274 | Vβ without signal | GVTQTPKFRVLKTGQSMTLLCAQDMNHEYMYWYRQDPGMGL |
| | peptide (SignalP) | RLIHYSVGEGTTAKGEVPDGYNVSRLKKQNFLLGLESAAPS |
| | | QTSVYFCASSYKLAGDNEQFFGPGTRLTVL |
| 275 | Vβ (without the | MSLGLLCCGAFSLLWAGPVNAGVTQTPKFRVLKTGQSMTLL |
| | Constant) | CAQDMNHEYMYWYRQDPGMGLRLIHYSVGEGTTAKGEVPDG |
| | | YNVSRLKKQNFLLGLESAAPSQTSVYFCASSYKLAGDNEQF |
| | | FGPGTRLTVL |
| 276 | β chain with WT | MSLGLLCCGAFSLLWAGPVNAGVTQTPKFRVLKTGQSMTLL |
| | signal peptide and | CAQDMNHEYMYWYRQDPGMGLRLIHYSVGEGTTAKGEVPDG |
| | constant Cβ | YNVSRLKKQNFLLGLESAAPSQTSVYFCASSYKLAGDNEQF |
| | | FGPGTRLTVLEDLRNVTPPKVSLFEPSKAEIANKQKATLVC |
| | | LARGFFPDHVELSWWVNGKEVHSGVSTDPQAYKESNYSYCL |
| | | SSRLRVSATFWHNPRNHFRCQVQFHGLSEEDKWPEGSPKPV |
| | | TQNISAEAWGRADCGITSASYQQGVLSATILYEILLGKATL |
| | | YAVLVSTLVVMAMVKRKNS |

In some embodiments, 0025-TCR77 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR77 interacts with and/or is specific for the neoantigen in the context of HLA-DRA and DRB1*01:01.

TABLE 24

| SEQ ID NO. | Description | 0025-TCR87 |
|---|---|---|
| 277 | CDR1α | YGATPY |
| 278 | CDR2α | YFSGDTLV |
| 279 | CDR3α | AVGRNTPLV |
| 280 | Vα without signal | QSVTQPDIHITVSEGASLELRCNYSYGATPYLFWYVQSPGQ |
| | peptide (SignalP) | GLQLLLKYFSGDTLVQGIKGFEAEFKRSQSSFNLRKPSVHW |
| | | SDAAEYFCAVGRNTPLVFGKGTRLSVIA |
| 281 | Vα only (without | MLLELIPLLGIHFVLRTARAQSVTQPDIHITVSEGASLELR |
| | the Constant) | CNYSYGATPYLFWYVQSPGQGLQLLLKYFSGDTLVQGIKGF |
| | | EAEFKRSQSSFNLRKPSVHWSDAAEYFCAVGRNTPLVFGKG |
| | | TRLSVIA |
| 282 | α chain with WT | MLLELIPLLGIHFVLRTARAQSVTQPDIHITVSEGASLELR |
| | signal peptide and | CNYSYGATPYLFWYVQSPGQGLQLLLKYFSGDTLVQGIKGF |
| | constant Cα | EAEFKRSQSSFNLRKPSVHWSDAAEYFCAVGRNTPLVFGKG |
| | | TRLSVIANIQNPEPAVYQLKDPRSQDSTLCLFTDFDSQINV |
| | | PKTMESGTFITDKTVLDMKAMDSKSNGAIAWSNQTSFTCQD |
| | | IFKETNATYPSSDVPCDATLTEKSFETDMNLNFQNLLVIVL |
| | | RILLLKVAGFNLLMTLRLWSS |
| 283 | CDR1β | SGHTA |
| 284 | CDR2β | FQGNSA |
| 285 | CDR3β | ASSSGGAFDRSGNTIY |
| 286 | Vβ without signal | GVSQSPSNKVTEKGKDVELRCDPISGHTALYWYRQSLGQGL |
| | peptide (SignalP) | EFLIYFQGNSAPDKSGLPSDRFSAERTGGSVSTLTIQRTQQ |
| | | EDSAVYLCASSSGGAFDRSGNTIYFGEGSWLTVV |
| 287 | Vβ (without the | MGTRLLFWVAFCLLGADHTGAGVSQSPSNKVTEKGKDVELR |
| | Constant) | CDPISGHTALYWYRQSLGQGLEFLIYFQGNSAPDKSGLPSD |
| | | RFSAERTGGSVSTLTIQRTQQEDSAVYLCASSSGGAFDRSG |
| | | NTIYFGEGSWLTVV |
| 288 | β chain with WT | MGTRLLFWVAFCLLGADHTGAGVSQSPSNKVTEKGKDVELR |
| | signal peptide and | CDPISGHTALYWYRQSLGQGLEFLIYFQGNSAPDKSGLPSD |
| | constant Cβ | RFSAERTGGSVSTLTIQRTQQEDSAVYLCASSSGGAFDRSG |
| | | NTIYFGEGSWLTVVEDLRNVTPPKVSLFEPSKAEIANKQKA |
| | | TLVCLARGFFPDHVELSWWVNGKEVHSGVSTDPQAYKESNY |
| | | SYCLSSRLRVSATFWHNPRNHFRCQVQFHGLSEEDKWPEGS |
| | | PKPVTQNISAEAWGRADCGITSASYQQGVLSATILYEILLG |
| | | KATLYAVLVSTLVVMAMVKRKNS |

HLA-DRA and In some embodiments, 0025-TCR87 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR87 interacts with and/or is specific for the neoantigen in the context of HLA-DRA and DRB1*01:01.

TABLE 25

| SEQ ID NO. | Description | 0025-TCR101 |
|---|---|---|
| 289 | CDR1α | YGATPY |
| 290 | CDR2α | YFSGDTLV |
| 291 | CDR3α | AGRGGGFKTI |
| 292 | Vα without signal | QSVTQPDIHITVSEGASLELRCNYSYGATPYLFWYVQSPGQ |
| | peptide (SignalP) | GLQLLLKYFSGDTLVQGIKGFEAEFKRSQSSFNLRKPSVHW |
| | | SDAAEYFCAGRGGGFKTIFGAGTRLFVKA |
| 293 | Vα only (without | MLLELIPLLGIHFVLRTARAQSVTQPDIHITVSEGASLELR |
| | the Constant) | CNYSYGATPYLFWYVQSPGQGLQLLLKYFSGDTLVQGIKGF |
| | | EAEFKRSQSSFNLRKPSVHWSDAAEYFCAGRGGGFKTIFGA |
| | | GTRLFVKA |
| 294 | α chain with WT | MLLELIPLLGIHFVLRTARAQSVTQPDIHITVSEGASLELR |
| | signal peptide and | CNYSYGATPYLFWYVQSPGQGLQLLLKYFSGDTLVQGIKGF |
| | constant Cα | EAEFKRSQSSFNLRKPSVHWSDAAEYFCAGRGGGFKTIFGA |
| | | GTRLFVKANIQNPEPAVYQLKDPRSQDSTLCLFTDFDSQIN |
| | | VPKTMESGTFITDKTVLDMKAMDSKSNGAIAWSNQTSFTCQ |
| | | DIFKETNATYPSSDVPCDATLTEKSFETDMNLNFQNLLVIV |
| | | LRILLLKVAGFNLLMTLRLWSS |
| 295 | CDR1β | LGHDT |
| 296 | CDR2β | YNNKEL |
| 297 | CDR3β | ASSSRLAGAQETQY |
| 298 | Vβ without signal | QTPKYLVTQMGNDKSIKCEQNLGHDTMYWYKQDSKKFLKIM |
| | peptide (SignalP) | FSYNNKELIINETVPNRFSPKSPDKAHLNLHINSLELGDSA |
| | | VYFCASSSRLAGAQETQYFGPGTRLLVL |
| 299 | Vβ (without the | MGCRLLCCVVFCLLQAGPLDTAVSQTPKYLVTQMGNDKSIK |
| | Constant) | CEQNLGHDTMYWYKQDSKKFLKIMFSYNNKELIINETVPNR |
| | | FSPKSPDKAHLNLHINSLELGDSAVYFCASSSRLAGAQETQ |
| | | YFGPGTRLLVL |
| 300 | β chain with WT | MGCRLLCCVVFCLLQAGPLDTAVSQTPKYLVTQMGNDKSIK |
| | signal peptide and | CEQNLGHDTMYWYKQDSKKFLKIMFSYNNKELIINETVPNR |
| | constant Cβ | FSPKSPDKAHLNLHINSLELGDSAVYFCASSSRLAGAQETQ |
| | | YFGPGTRLLVLEDLRNVTPPKVSLFEPSKAEIANKQKATLV |
| | | CLARGFFPDHVELSWWVNGKEVHSGVSTDPQAYKESNYSYC |
| | | LSSRLRVSATFWHNPRNHFRCQVQFHGLSEEDKWPEGSPKP |
| | | VTQNISAEAWGRADCGITSASYQQGVLSATILYEILLGKAT |
| | | LYAVLVSTLVVMAMVKRKNS |

In some embodiments, 0025-TCR101 interacts with and/or is specific for a peptide from gene RHPN2. In some embodiments, the peptide is from a neoantigen of RHPN2 and has the amino acid change S201C (in which position 201 of the RHPN2 protein is mutated from Ser to Cys). In some embodiments, 0025-TCR101 interacts with and/or is specific for the neoantigen in the context of HLA-DRA and DRB1*01:01.

TABLE 26

| SEQ ID | | |
|---|---|---|
| NO. | Description | 8540-TCR20 |
| 536 | CDR1α | VSGNPY |
| 537 | CDR2α | YITGDNLV |
| 538 | CDR3α | AVSLFLDDKII |
| 539 | Vα without signal | QSVAQPEDQVNVAEGNPLTVKCTYSVSGNPYLFWYVQYP |
| | peptide (SignalP) | NRGLQFLLKYITGDNLVKGSYGFEAEENKSQTSFHLKKP |
| | | SALVSDSALYFCAVSLFLDDKIIFGKGTRLHILPNIQNP |
| | | EPAV |
| 540 | Vα only (without | MASAPISMLAMLFTLSGLRAQSVAQPEDQVNVAEGNPLT |
| | the Constant) | VKCTYSVSGNPYLFWYVQYPNRGLQFLLKYITGDNLVKG |
| | | SYGFEAEFNKSQTSFHLKKPSALVSDSALYFCAVSLFLD |
| | | DKIIFGKGTRLHILPNIQNPEPAV |
| 541 | α chain with WT | MASAPISMLAMLFTLSGLRAQSVAQPEDQVNVAEGNPLT |
| | signal peptide | VKCTYSVSGNPYLFWYVQYPNRGLQFLLKYITGDNLVKG |
| | and constant Cα | SYGFEAEFNKSQTSFHLKKPSALVSDSALYFCAVSLFLD |
| | | DKIIFGKGTRLHILPNIQNPEPAVNIQNPEPAVYQLKDP |
| | | RSQDSTLCLFTDFDSQINVPKTMESGTFITDKTVLDMKA |
| | | MDSKSNGAIAWSNQTSFTCQDIFKETNATYPSSDVPCDA |
| | | TLTEKSFETDMNLNFQNLLVIVLRILLLKVAGFNLLMTL |
| | | RLWSS |
| 542 | CDR1β | SGHRS |
| 543 | CDR2β | YFSETQ |
| 544 | CDR3β | ASSLARVEDEAF |
| 545 | Vβ without signal | GVTQTPRYLIKTRGQQVTLSCSPISGHRSVSWYQQTPGQ |
| | peptide (SignalP) | GLQFLFEYFSETQRNKGNFPGRESGRQFSNSRSEMNVST |
| | | LELGDSALYLCASSLARVEDEAFFGQGTRLTVVEDLRN |
| 546 | Vβ (without the | MGSRLLCWVLLCLLGAGPVKAGVTQTPRYLIKTRGQQVT |
| | Constant) | LSCSPISGHRSVSWYQQTPGQGLQFLFEYFSETQRNKGN |
| | | FPGRESGRQFSNSRSEMNVSTLELGDSALYLCASSLARV |
| | | EDEAFFGQGTRLTVVEDLRN |
| 547 | β chain with WT | MGSRLLCWVLLCLLGAGPVKAGVTQTPRYLIKTRGQQVT |
| | signal peptide | LSCSPISGHRSVSWYQQTPGQGLQFLFEYFSETQRNKGN |
| | and constant Cβ | FPGRFSGRQFSNSRSEMNVSTLELGDSALYLCASSLARV |
| | | EDEAFFGQGTRLTVVEDLRNEDLRNVTPPKVSLFEPSKA |
| | | EIANKQKATLVCLARGFFPDHVELSWWVNGKEVHSGVST |
| | | DPQAYKESNYSYCLSSRLRVSATFWHNPRNHFRCQVQFH |
| | | GLSEEDKWPEGSPKPVTQNISAEAWGRADCGITSASYQQ |
| | | GVLSATILYEILLGKATLYAVLVSTLVVMAMVKRKNS |

In some embodiments, 8540-TCR20 interacts with and/or is specific for a peptide from gene NUP205. In some embodiments, the peptide is from a neoantigen of NUP205 and has the amino acid change R214H (in which position 214 of the NUP205 protein is mutated from Arg to His). In some embodiments, 8540-TCR20 interacts with and/or is specific for the neoantigen in the context of HLA-B*38:01.

TABLE 27

| SEQ ID | | |
|---|---|---|
| NO. | Description | 8540-TCR22-2 |
| 548 | CDR1α | VSGNPY |
| 549 | CDR2α | YITGDNLV |
| 550 | CDR3α | AVRGFLINDMR |
| 551 | Vα without signal | QSVAQPEDQVNVAEGNPLTVKCTYSVSGNPYLFWYVQYP |
| | peptide (SignalP) | NRGLQFLLKYITGDNLVKGSYGFEAEFNKSQTSFHLKKP |
| | | SALVSDSALYFCAVRGFLINDMRFGAGTRLTVKPNIQNP |
| | | EPAV |
| 552 | Vα only (without | MASAPISMLAMLFTLSGLRAQSVAQPEDQVNVAEGNPLT |
| | the Constant) | VKCTYSVSGNPYLFWYVQYPNRGLQFLLKYITGDNLVKG |
| | | SYGFEAEENKSQTSFHLKKPSALVSDSALYFCAVRGFLI |
| | | NDMRFGAGTRLTVKPNIQNPEPAV |
| 553 | α chain with WT | MASAPISMLAMLFTLSGLRAQSVAQPEDQVNVAEGNPLT |
| | signal peptide | VKCTYSVSGNPYLFWYVQYPNRGLQFLLKYITGDNLVKG |
| | and constant Cα | SYGFEAEFNKSQTSFHLKKPSALVSDSALYFCAVRGFLI |
| | | NDMRFGAGTRLTVKPNIQNPEPAVNIQNPEPAVYQLKDP |
| | | RSQDSTLCLFTDFDSQINVPKTMESGTFITDKTVLDMKA |
| | | MDSKSNGAIAWSNQTSFTCQDIFKETNATYPSSDVPCDA |
| | | TLTEKSFETDMNLNFQNLLVIVLRILLLKVAGFNLLMTL |
| | | RLWSS |
| 554 | CDR1β | SGHRS |
| 555 | CDR2β | YFSETQ |
| 556 | CDR3β | ASSLGRVENEQY |
| 557 | Vβ without signal | GVTQTPRYLIKTRGQQVTLSCSPISGHRSVSWYQQTPGQ |
| | peptide (SignalP) | GLQFLFEYFSETQRNKGNFPGRFSGRQFSNSRSEMNVST |
| | | LELGDSALYLCASSLGRVENEQYFGPGTRLTVTEDLRN |
| 558 | Vβ (without the | MGSRLLCWVLLCLLGAGPVKAGVTQTPRYLIKTRGQQVT |
| | Constant) | LSCSPISGHRSVSWYQQTPGQGLQFLFEYFSETQRNKGN |
| | | FPGRFSGRQFSNSRSEMNVSTLELGDSALYLCASSLGRV |
| | | ENEQYFGPGTRLTVTEDLRN |
| 559 | β chain with WT | MGSRLLCWVLLCLLGAGPVKAGVTQTPRYLIKTRGQQVT |
| | signal peptide | LSCSPISGHRSVSWYQQTPGQGLQFLFEYFSETQRNKGN |
| | and constant Cβ | FPGRFSGRQFSNSRSEMNVSTLELGDSALYLCASSLGRV |
| | | ENEQYFGPGTRLTVTEDLRNEDLRNVTPPKVSLFEPSKA |
| | | EIANKQKATLVCLARGFFPDHVELSWWVNGKEVHSGVST |
| | | DPQAYKESNYSYCLSSRLRVSATFWHNPRNHFRCQVQFH |
| | | GLSEEDKWPEGSPKPVTQNISAEAWGRADCGITSASYQQ |
| | | GVLSATILYEILLGKATLYAVLVSTLVVMAMVKRKNS |

In some embodiments, 8540-TCR22-2 interacts with and/or is specific for a peptide from gene NUP205. In some embodiments, the peptide is from a neoantigen of NUP205 and has the amino acid change R214H (in which position 214 of the NUP205 protein is mutated from Arg to His). In some embodiments, 8540-TCR22-2 interacts with and/or is specific for the neoantigen in the context of HLA-B*38:01.

TABLE 28

| SEQ ID | | |
|---|---|---|
| NO. | Description | 8540-TCR56 |
| 560 | CDR1α | SSVSVY |
| 561 | CDR2α | YLSGSTLV |
| 562 | CDR3α | AVMERGGSNYKLT |
| 563 | Vα without signal | QSVTQLDSQVPVFEEAPVELRCNYSSSVSVYLFWYVQYP |
| | peptide (SignalP) | NQGLQLLLKYLSGSTLVKGINGFEAEENKSQTSFHLRKP |
| | | SVHISDTAEYFCAVMERGGSNYKLTFGKGTLLTVNPNIQ |
| | | NPEPAV |
| 564 | Vα only (without | MLLLLVPAFQVIFTLGGTRAQSVTQLDSQVPVFEEAPVE |
| | the Constant) | LRCNYSSSVSVYLFWYVQYPNQGLQLLLKYLSGSTLVKG |
| | | INGFEAEFNKSQTSFHLRKPSVHISDTAEYFCAVMERGG |
| | | SNYKLTFGKGTLLTVNPNIQNPEPAV |
| 565 | α chain with WT | MLLLLVPAFQVIFTLGGTRAQSVTQLDSQVPVFEEAPVE |
| | signal peptide | LRCNYSSSVSVYLFWYVQYPNQGLQLLLKYLSGSTLVKG |
| | and constant Cα | INGFEAEFNKSQTSFHLRKPSVHISDTAEYFCAVMERGG |
| | | SNYKLTFGKGTLLTVNPNIQNPEPAVNIQNPEPAVYQLK |
| | | DPRSQDSTLCLFTDFDSQINVPKTMESGTFITDKTVLDM |
| | | KAMDSKSNGAIAWSNQTSFTCQDIFKETNATYPSSDVPC |
| | | DATLTEKSFETDMNLNFQNLLVIVLRILLLKVAGFNLLM |
| | | TLRLWSS |
| 566 | CDR1β | LNHDA |
| 567 | CDR2β | SQIVND |
| 568 | CDR3β | ASSRDGYPGNTIY |
| 569 | Vβ without signal | GITQSPKYLFRKEGQNVTLSCEQNLNHDAMYWYRQDPGQ |
| | peptide (SignalP) | GLRLIYYSQIVNDFQKGDIAEGYSVSREKKESFPLTVTS |
| | | AQKNPTAFYLCASSRDGYPGNTIYFGEGSWLTVVEDLRN |
| 570 | Vβ (without the | MSNQVLCCVVLCLLGANTVDGGITQSPKYLFRKEGQNVT |
| | Constant) | LSCEQNLNHDAMYWYRQDPGQGLRLIYYSQIVNDFQKGD |
| | | IAEGYSVSREKKESFPLTVTSAQKNPTAFYLCASSRDGY |
| | | PGNTIYFGEGSWLTVVEDLRN |
| 571 | β chain with WT | MSNQVLCCVVLCLLGANTVDGGITQSPKYLFRKEGQNVT |
| | signal peptide | LSCEQNLNHDAMYWYRQDPGQGLRLIYYSQIVNDFQKGD |
| | and constant Cβ | IAEGYSVSREKKESFPLTVTSAQKNPTAFYLCASSRDGY |
| | | PGNTIYFGEGSWLTVVEDLRNEDLRNVTPPKVSLFEPSK |
| | | AEIANKQKATLVCLARGFFPDHVELSWWVNGKEVHSGVS |
| | | TDPQAYKESNYSYCLSSRLRVSATFWHNPRNHFRCQVQF |
| | | HGLSEEDKWPEGSPKPVTQNISAEAWGRADCGITSASYQ |
| | | QGVLSATILYEILLGKATLYAVLVSTLVVMAMVKRKNS |

In some embodiments, 8540-TCR56 interacts with and/or is specific for a peptide from gene NUP205. In some embodiments, the peptide is from a neoantigen of NUP205 and has the amino acid change R214H (in which position 214 of the NUP205 protein is mutated from Arg to His). In some embodiments, 8540-TCR56 interacts with and/or is specific for the neoantigen in the context of HLA-B*38:01.

TABLE 29

| SEQ ID | | |
|---|---|---|
| NO. | Description | 8540-TCR33 |
| 572 | CDR1α | TISGNEY |
| 573 | CDR2α | GLKNN |
| 574 | CDR3α | IVRPHNTGKLI |
| 575 | Vα without signal | KTTQPPSMDCAEGRAANLPCNHSTISGNEYVYWYRQIHS |
| | peptide (SignalP) | QGPQYIIHGLKNNETNEMASLIITEDRKSSTLILPHATL |
| | | RDTAVYYCIVRPHNTGKLIFGQGTTLQVKPNIQNPEPAV |
| 576 | Vα only (without | MRLVARVTVFLTFGTIIDAKTTQPPSMDCAEGRAANLPC |
| | the Constant) | NHSTISGNEYVYWYRQIHSQGPQYIIHGLKNNETNEMAS |
| | | LIITEDRKSSTLILPHATLRDTAVYYCIVRPHNTGKLIF |
| | | GQGTTLQVKPNIQNPEPAV |
| 577 | α chain with WT | MRLVARVTVFLTFGTIIDAKTTQPPSMDCAEGRAANLPC |
| | signal peptide | NHSTISGNEYVYWYRQIHSQGPQYIIHGLKNNETNEMAS |
| | and constant Cα | LIITEDRKSSTLILPHATLRDTAVYYCIVRPHNTGKLIF |
| | | GQGTTLQVKPNIQNPEPAVNIQNPEPAVYQLKDPRSQDS |
| | | TLCLFTDFDSQINVPKTMESGTFITDKTVLDMKAMDSKS |
| | | NGAIAWSNQTSFTCQDIFKETNATYPSSDVPCDATLTEK |
| | | SFETDMNLNFQNLLVIVLRILLLKVAGFNLLMTLRLWSS |
| 578 | CDR1β | MNHNS |
| 579 | CDR2β | SASEGT |
| 580 | CDR3β | ASSEMDSGTDTQY |
| 581 | Vβ without signal | GVTQTPKFQVLKTGQSMTLQCAQDMNHNSMYWYRQDPGM |
| | peptide (SignalP) | GLRLIYYSASEGTTDKGEVPNGYNVSRLNKREFSLRLES |
| | | AAPSQTSVYFCASSEMDSGTDTQYFGPGTRLTVLEDLRN |
| 582 | Vβ (without the | MSIGLLCCVAFSLLWASPVNAGVTQTPKFQVLKTGQSMT |
| | Constant) | LQCAQDMNHNSMYWYRQDPGMGLRLIYYSASEGTTDKGE |
| | | VPNGYNVSRLNKREFSLRLESAAPSQTSVYFCASSEMDS |
| | | GTDTQYFGPGTRLTVLEDLRN |
| 583 | β chain with WT | MSIGLLCCVAFSLLWASPVNAGVTQTPKFQVLKTGQSMT |
| | signal peptide | LQCAQDMNHNSMYWYRQDPGMGLRLIYYSASEGTTDKGE |
| | and constant Cβ | VPNGYNVSRLNKREFSLRLESAAPSQTSVYFCASSEMDS |
| | | GTDTQYFGPGTRLTVLEDLRNEDLRNVTPPKVSLFEPSK |
| | | AEIANKQKATLVCLARGFFPDHVELSWWVNGKEVHSGVS |
| | | TDPQAYKESNYSYCLSSRLRVSATFWHNPRNHFRCQVQF |
| | | HGLSEEDKWPEGSPKPVTQNISAEAWGRADCGITSASYQ |
| | | QGVLSATILYEILLGKATLYAVLVSTLVVMAMVKRKNS |

In some embodiments, 8540-TCR33 interacts with and/or is specific for a peptide from gene PCSK9. In some embodiments, the peptide is from a neoantigen of PCSK9 and has the amino acid change C477Y (in which position 477 of the PCSK9 protein is mutated from Cys to Tyr). In some embodiments, 8540-TCR33 interacts with and/or is specific for the neoantigen in the context of DQA1*01:03 and DQB1*06:03.

TABLE 30

| SEQ ID | | |
|---|---|---|
| NO. | Description | 8540-TCR83 |
| 584 | CDR1α | TSESNYY |
| 585 | CDR2α | QEAYKQQN |
| 586 | CDR3α | ALKETSGSRLT |
| 587 | Vα without signal | QTVTQSQPEMSVQEAETVTLSCTYDTSESNYYLFWYKQP |
| | peptide (SignalP) | PSRQMILVIRQEAYKQQNATENRFSVNFQKAAKSFSLKI |
| | | SDSQLGDTAMYFCALKETSGSRLTFGEGTQLTVNPNIQN |
| | | PEPAV |
| 588 | Vα only (without | MTRVSLLWAVVVSTCLESGMAQTVTQSQPEMSVQEAETV |
| | the Constant) | TLSCTYDTSESNYYLFWYKQPPSRQMILVIRQEAYKQQN |
| | | ATENRFSVNFQKAAKSFSLKISDSQLGDTAMYFCALKET |
| | | SGSRLTFGEGTQLTVNPNIQNPEPAV |
| 589 | α chain with WT | MTRVSLLWAVVVSTCLESGMAQTVTQSQPEMSVQEAETV |
| | signal peptide | TLSCTYDTSESNYYLFWYKQPPSRQMILVIRQEAYKQQN |
| | and constant Cα | ATENRFSVNFQKAAKSFSLKISDSQLGDTAMYFCALKET |
| | | SGSRLTFGEGTQLTVNPNIQNPEPAVNIQNPEPAVYQLK |
| | | DPRSQDSTLCLFTDFDSQINVPKTMESGTFITDKTVLDM |
| | | KAMDSKSNGAIAWSNQTSFTCQDIFKETNATYPSSDVPC |
| | | DATLTEKSFETDMNLNFQNLLVIVLRILLLKVAGFNLLM |
| | | TLRLWSS |
| 590 | CDR1β | MGHRA |
| 591 | CDR2β | YSYEKL |
| 592 | CDR3β | ASSQDNTYNEQF |
| 593 | Vβ without signal | EVTQTPKHLVMGMTNKKSLKCEQHMGHRAMYWYKQKAKK |
| | peptide (SignalP) | PPELMFVYSYEKLSINESVPSRFSPECPNSSLLNLHLHA |
| | | LQPEDSALYLCASSQDNTYNEQFFGPGTRLTVLEDLRN |
| 594 | Vβ (without the | MGCRLLCCAVLCLLGAVPIDTEVTQTPKHLVMGMTNKKS |
| | Constant) | LKCEQHMGHRAMYWYKQKAKKPPELMFVYSYEKLSINES |
| | | VPSRFSPECPNSSLLNLHLHALQPEDSALYLCASSQDNT |
| | | YNEQFFGPGTRLTVLEDLRN |
| 595 | β chain with WT | MGCRLLCCAVLCLLGAVPIDTEVTQTPKHLVMGMTNKKS |
| | signal peptide | LKCEQHMGHRAMYWYKQKAKKPPELMFVYSYEKLSINES |
| | and constant Cβ | VPSRFSPECPNSSLLNLHLHALQPEDSALYLCASSQDNT |
| | | YNEQFFGPGTRLTVLEDLRNEDLRNVTPPKVSLFEPSKA |
| | | EIANKQKATLVCLARGFFPDHVELSWWVNGKEVHSGVST |
| | | DPQAYKESNYSYCLSSRLRVSATFWHNPRNHFRCQVQFH |
| | | GLSEEDKWPEGSPKPVTQNISAEAWGRADCGITSASYQQ |
| | | GVLSATILYEILLGKATLYAVLVSTLVVMAMVKRKNS |

In some embodiments, 8540-TCR83 interacts with and/or is specific for a peptide from gene PCSK9. In some embodiments, the peptide is from a neoantigen of PCSK9 and has the amino acid change C477Y (in which position 477 of the PCSK9 protein is mutated from Cys to Tyr). In some embodiments, 8540-TCR83 interacts with and/or is specific for the neoantigen in the context of DQA1*01:03 and DQB1*06:03.

TABLE 31

| SEQ ID | | |
|---|---|---|
| NO. | Description | 8540-TCR26 |
| 596 | CDR1α | TISGNEY |
| 597 | CDR2α | GLKNN |
| 598 | CDR3α | IVRAHNDYKLS |
| 599 | Vα without signal | KTTQPPSMDCAEGRAANLPCNHSTISGNEYVYWYRQIHS |
| | peptide (SignalP) | QGPQYIIHGLKNNETNEMASLIITEDRKSSTLILPHATL |
| | | RDTAVYYCIVRAHNDYKLSFGAGTTVTVRANIQNPEPAV |
| 600 | Vα only (without | MRLVARVTVFLTFGTIIDAKTTQPPSMDCAEGRAANLPC |
| | the Constant) | NHSTISGNEYVYWYRQIHSQGPQYIIHGLKNNETNEMAS |
| | | LIITEDRKSSTLILPHATLRDTAVYYCIVRAHNDYKLSF |
| | | GAGTTVTVRANIQNPEPAV |
| 601 | α chain with WT | MRLVARVTVFLTFGTIIDAKTTQPPSMDCAEGRAANLPC |
| | signal peptide | NHSTISGNEYVYWYRQIHSQGPQYIIHGLKNNETNEMAS |
| | and constant Cα | LIITEDRKSSTLILPHATLRDTAVYYCIVRAHNDYKLSF |
| | | GAGTTVTVRANIQNPEPAVNIQNPEPAVYQLKDPRSQDS |
| | | TLCLFTDFDSQINVPKTMESGTFITDKTVLDMKAMDSKS |
| | | NGAIAWSNQTSFTCQDIFKETNATYPSSDVPCDATLTEK |
| | | SFETDMNLNFQNLLVIVLRILLLKVAGENLLMTLRLWSS |
| 602 | CDR1β | MNHNS |
| 603 | CDR2β | SASEGT |
| 604 | CDR3β | ASSPDVGDYGYT |
| 605 | Vβ without signal | GVTQTPKFQVLKTGQSMTLQCAQDMNHNSMYWYRQDPGM |
| | peptide (SignalP) | GLRLIYYSASEGTTDKGEVPNGYNVSRLNKREFSLRLES |
| | | AAPSQTSVYFCASSPDVGDYGYTFGSGTRLTVVEDLRN |
| 606 | Vβ (without the | MSIGLLCCVAFSLLWASPVNAGVTQTPKFQVLKTGQSMT |
| | Constant) | LQCAQDMNHNSMYWYRQDPGMGLRLIYYSASEGTTDKGE |
| | | VPNGYNVSRLNKREFSLRLESAAPSQTSVYFCASSPDVG |
| | | DYGYTFGSGTRLTVVEDLRN |
| 607 | β chain with WT | MSIGLLCCVAFSLLWASPVNAGVTQTPKFQVLKTGQSMT |
| | signal peptide | LQCAQDMNHNSMYWYRQDPGMGLRLIYYSASEGTTDKGE |
| | and constant Cβ | VPNGYNVSRLNKREFSLRLESAAPSQTSVYFCASSPDVG |
| | | DYGYTFGSGTRLTVVEDLRNEDLRNVTPPKVSLFEPSKA |
| | | EIANKQKATLVCLARGFFPDHVELSWWVNGKEVHSGVST |
| | | DPQAYKESNYSYCLSSRLRVSATFWHNPRNHFRCQVQFH |
| | | GLSEEDKWPEGSPKPVTQNISAEAWGRADCGITSASYQQ |
| | | GVLSATILYEILLGKATLYAVLVSTLVVMAMVKRKNS |

In some embodiments, 8540-TCR26 interacts with and/or is specific for a peptide from gene PCSK9. In some embodiments, the peptide is from a neoantigen of PCSK9 and has the amino acid change C477Y (in which position 477 of the PCSK9 protein is mutated from Cys to Tyr). In some embodiments, 8540-TCR26 interacts with and/or is specific for the neoantigen in the context of DQA1*01:03 and DQB1*06:03.

TABLE 32

| SEQ ID | | |
|---|---|---|
| NO. | Description | 8540-TCR25 |
| 608 | CDR1α | SSVSVY |
| 609 | CDR2α | YLSGSTLV |
| 610 | CDR3α | AVSDHGFGNEKLT |
| 611 | Vα without signal | QSVTQLDSQVPVFEEAPVELRCNYSSSVSVYLFWYVQYP |
| | peptide (SignalP) | NQGLQLLLKYLSGSTLVKGINGFEAEFNKSQTSFHLRKP |
| | | SVHISDTAEYFCAVSDHGFGNEKLTFGTGTRLTIIPNIQ |
| | | NPEPAV |
| 612 | Vα only (without | MLLLLVPAFQVIFTLGGTRAQSVTQLDSQVPVFEEAPVE |
| | the Constant) | LRCNYSSSVSVYLFWYVQYPNQGLQLLLKYLSGSTLVKG |
| | | INGFEAEFNKSQTSFHLRKPSVHISDTAEYFCAVSDHGF |
| | | GNEKLTFGTGTRLTIIPNIQNPEPAV |
| 613 | α chain with WT | MLLLLVPAFQVIFTLGGTRAQSVTQLDSQVPVFEEAPVE |
| | signal peptide | LRCNYSSSVSVYLFWYVQYPNQGLQLLLKYLSGSTLVKG |
| | and constant Cα | INGFEAEFNKSQTSFHLRKPSVHISDTAEYFCAVSDHGF |
| | | GNEKLTFGTGTRLTIIPNIQNPEPAVNIQNPEPAVYQLK |
| | | DPRSQDSTLCLFTDFDSQINVPKTMESGTFITDKTVLDM |
| | | KAMDSKSNGAIAWSNQTSFTCQDIFKETNATYPSSDVPC |
| | | DATLTEKSFETDMNLNFQNLLVIVLRILLLKVAGENLLM |
| | | TLRLWSS |
| 614 | CDR1β | DFQATT |
| 615 | CDR2β | SNEGSKA |
| 616 | CDR3β | SARRDRNQPQH |
| 617 | Vβ without signal | AVVSQHPSRVICKSGTSVKIECRSLDFQATTMFWYRQFP |
| | peptide (SignalP) | KQSLMLMATSNEGSKATYEQGVEKDKELINHASLTLSTL |
| | | TVTSAHPEDSSFYICSARRDRNQPQHFGDGIRLSILEDL |
| | | RN |
| 618 | Vβ (without the Constant) | MLLLLLLLGPGSGLGAVVSQHPSRVICKSGTSVKIECRS |
| | | LDFQATTMFWYRQFPKQSLMLMATSNEGSKATYEQGVEK |
| | | DKFLINHASLTLSTLTVTSAHPEDSSFYICSARRDRNQP |
| | | QHFGDGIRLSILEDLRN |
| 619 | β chain with WT | MLLLLLLLGPGSGLGAVVSQHPSRVICKSGTSVKIECRS |
| | signal peptide | LDFQATTMFWYRQFPKQSLMLMATSNEGSKATYEQGVEK |
| | and constant Cβ | DKFLINHASLTLSTLTVTSAHPEDSSFYICSARRDRNQP |
| | | QHFGDGIRLSILEDLRNEDLRNVTPPKVSLFEPSKAEIA |
| | | NKQKATLVCLARGFFPDHVELSWWVNGKEVHSGVSTDPQ |
| | | AYKESNYSYCLSSRLRVSATFWHNPRNHFRCQVQFHGLS |
| | | EEDKWPEGSPKPVTQNISAEAWGRADCGITSASYQQGVL |
| | | SATILYEILLGKATLYAVLVSTLVVMAMVKRKNS |

In some embodiments, 8540-TCR25 interacts with and/or is specific for a peptide from gene CEP85. In some embodiments, the peptide is from a neoantigen of CEP85 and has the amino acid change H549R (in which position 549 of the CEP85 protein is mutated from His to Arg). In some embodiments, 8540-TCR25 interacts with and/or is specific for the neoantigen in the context of HLA-DRA and DRB1*11:01.

TABLE 33

| SEQ ID | | |
|---|---|---|
| NO. | Description | 0894-TCR43 |
| 620 | CDR1α | NSMFDY |
| 621 | CDR2α | ISSIKDK |
| 622 | CDR3α | AARSGTYKYI |
| 623 | Vα without signal peptide | QQKNDDQQVKQNSPSLSVQEGRISILNCDYTNSMEDYFL |
| | (SignalP) | WYKKYPAEGPTFLISISSIKDKNEDGRFTVFLNKSAKHL |
| | | SLHIVPSQPGDSAVYFCAARSGTYKYIFGTGTRLKVL |
| 624 | Vα only (without | MAMLLGASVLILWLQPDWVNSQQKNDDQQVKQNSPSLSV |
| | the Constant) | QEGRISILNCDYTNSMFDYFLWYKKYPAEGPTFLISISS |
| | | IKDKNEDGRFTVFLNKSAKHLSLHIVPSQPGDSAVYFCA |
| | | ARSGTYKYIFGTGTRLKVL |
| 625 | α chain with WT | MAMLLGASVLILWLQPDWVNSQQKNDDQQVKQNSPSLSV |
| | signal peptide | QEGRISILNCDYTNSMFDYFLWYKKYPAEGPTFLISISS |
| | and constant Cα | IKDKNEDGRFTVFLNKSAKHLSLHIVPSQPGDSAVYFCA |
| | | ARSGTYKYIFGTGTRLKVLNIQNPEPAVYQLKDPRSQDS |
| | | TLCLFTDFDSQINVPKTMESGTFITDKTVLDMKAMDSKS |
| | | NGAIAWSNQTSFTCQDIFKETNATYPSSDVPCDATLTEK |
| | | SFETDMNLNFQNLLVIVLRILLLKVAGENLLMTLRLWSS |
| 626 | CDR1β | SGDLS |
| 627 | CDR2β | YYNGEE |
| 628 | CDR3β | ASSEGVGQIYGYT |
| 629 | Vβ without signal | GVTQTPKHLITATGQRVTLRCSPRSGDLSVYWYQQSLDQ |
| | peptide (SignalP) | GLQFLIQYYNGEERAKGNILERFSAQQFPDLHSELNLSS |
| | | LELGDSALYFCASSEGVGQIYGYTFGSGTRLTVV |
| 630 | Vβ (without the Constant) | MGFRLLCCVAFCLLGAGPVDSGVTQTPKHLITATGQRVT |
| | | LRCSPRSGDLSVYWYQQSLDQGLQFLIQYYNGEERAKGN |
| | | ILERFSAQQFPDLHSELNLSSLELGDSALYFCASSEGVG |
| | | QIYGYTFGSGTRLTVV |
| 631 | β chain with WT | MGFRLLCCVAFCLLGAGPVDSGVTQTPKHLITATGQRVT |
| | signal peptide | LRCSPRSGDLSVYWYQQSLDQGLQFLIQYYNGEERAKGN |
| | and constant Cβ | ILERFSAQQFPDLHSELNLSSLELGDSALYFCASSEGVG |
| | | QIYGYTFGSGTRLTVVEDLRNVTPPKVSLFEPSKAEIAN |
| | | KQKATLVCLARGFFPDHVELSWWVNGKEVHSGVSTDPQA |
| | | YKESNYSYCLSSRLRVSATFWHNPRNHFRCQVQFHGLSE |
| | | EDKWPEGSPKPVTQNISAEAWGRADCGITSASYQQGVLS |
| | | ATILYEILLGKATLYAVLVSTLVVMAMVKRKNS |

In some embodiments, 0894-TCR43 interacts with and/or is specific for a peptide from gene HNRNPF. In some embodiments, the peptide is from a neoantigen of HNRNPF and has the amino acid change E56K (in which position 56 of the HNRNPF protein is mutated from Glu to Lys). In some embodiments, 0894-TCR43 interacts with and/or is specific for the neoantigen in the context of HLA-DRA and DRB1*11:01.

TABLE 34

| SEQ ID | | |
|---|---|---|
| NO. | Description | 0894-TCR63 |
| 632 | CDR1α | TISGTDY |
| 633 | CDR2α | GLTSN |
| 634 | CDR3α | ILFSGNTGKLI |
| 635 | Vα without signal | KTTQPNSMESNEEEPVHLPCNHSTISGTDYIHWYRQLPS |
| | peptide (SignalP) | QGPEYVIHGLTSNVNNRMASLAIAEDRKSSTLILHRATL |
| | | RDAAVYYCILFSGNTGKLIFGQGTTLQVKP |
| 636 | Vα only (without | MKLVTSITVLLSLGIMGDAKTTQPNSMESNEEEPVHLPC |
| | the Constant) | NHSTISGTDYIHWYRQLPSQGPEYVIHGLTSNVNNRMAS |
| | | LAIAEDRKSSTLILHRATLRDAAVYYCILFSGNTGKLIF |
| | | GQGTTLQVKP |
| 637 | α chain with WT | MKLVTSITVLLSLGIMGDAKTTQPNSMESNEEEPVHLPC |
| | signal peptide | NHSTISGTDYIHWYRQLPSQGPEYVIHGLTSNVNNRMAS |
| | and constant Cα | LAIAEDRKSSTLILHRATLRDAAVYYCILFSGNTGKLIF |
| | | GQGTTLQVKPNIQNPEPAVYQLKDPRSQDSTLCLFTDFD |
| | | SQINVPKTMESGTFITDKTVLDMKAMDSKSNGAIAWSNQ |
| | | TSFTCQDIFKETNATYPSSDVPCDATLTEKSFETDMNLN |
| | | FQNLLVIVLRILLLKVAGFNLLMTLRLWSS |
| 638 | CDR1β | LGHNA |
| 639 | CDR2β | YSLEER |
| 640 | CDR3β | ASSQDRGDLYGYT |
| 641 | Vβ without signal | GVTQTPRHLVMGMTNKKSLKCEQHLGHNAMYWYKQSAKK |
| | peptide (SignalP) | PLELMEVYSLEERVENNSVPSRFSPECPNSSHLFLHLHT |
| | | LQPEDSALYLCASSQDRGDLYGYTFGSGTRLTVV |
| 642 | Vβ (without the | MGCRLLCCAVLCLLGAVPMETGVTQTPRHLVMGMTNKKS |
| | Constant) | LKCEQHLGHNAMYWYKQSAKKPLELMFVYSLEERVENNS |
| | | VPSRFSPECPNSSHLFLHLHTLQPEDSALYLCASSQDRG |
| | | DLYGYTFGSGTRLTVV |
| 643 | β chain with WT | MGCRLLCCAVLCLLGAVPMETGVTQTPRHLVMGMTNKKS |
| | signal peptide | LKCEQHLGHNAMYWYKQSAKKPLELMFVYSLEERVENNS |
| | and constant Cβ | VPSRFSPECPNSSHLFLHLHTLQPEDSALYLCASSQDRG |
| | | DLYGYTFGSGTRLTVVEDLRNVTPPKVSLFEPSKAEIAN |
| | | KQKATLVCLARGFFPDHVELSWWVNGKEVHSGVSTDPQA |
| | | YKESNYSYCLSSRLRVSATFWHNPRNHFRCQVQFHGLSE |
| | | EDKWPEGSPKPVTQNISAEAWGRADCGITSASYQQGVLS |
| | | ATILYEILLGKATLYAVLVSTLVVMAMVKRKNS |

In some embodiments, 0894-TCR63 interacts with and/or is specific for a peptide from gene KDM1A. In some embodiments, the peptide is from a neoantigen of KDM1A and has the amino acid change D691H (in which position 691 of the KDM1A protein is mutated from Asp to His). In some embodiments, 0894-TCR63 interacts with and/or is specific for the neoantigen in the context of HLA-DRA and DRB1*14:54.

TABLE 35

| SEQ ID | | |
|---|---|---|
| NO. | Description | 0894-TCR92 |
| 644 | CDR1α | DSAIYN |
| 645 | CDR2α | IQSSQRE |
| 646 | CDR3α | APRGFGNVLH |
| 647 | Vα without signal | KQEVTQIPAALSVPEGENLVLNCSFTDSAIYNLQWFRQD |
| | peptide (SignalP) | PGKGLTSLLLIQSSQREQTSGRLNASLDKSSGRSTLYIA |
| | | ASQPGDSATYLCAPRGFGNVLHCGSGTQVIVLP |
| 648 | Vα only (without | METLLGLLILWLQLQWVSSKQEVTQIPAALSVPEGENLV |
| | the Constant) | LNCSFTDSAIYNLQWFRQDPGKGLTSLLLIQSSQREQTS |
| | | GRLNASLDKSSGRSTLYIAASQPGDSATYLCAPRGFGNV |
| | | LHCGSGTQVIVLP |
| 659 | α chain with WT | METLLGLLILWLQLQWVSSKQEVTQIPAALSVPEGENLV |
| | signal peptide | LNCSFTDSAIYNLQWFRQDPGKGLTSLLLIQSSQREQTS |
| | and constant Cα | GRLNASLDKSSGRSTLYIAASQPGDSATYLCAPRGFGNV |
| | | LHCGSGTQVIVLPNIQNPEPAVYQLKDPRSQDSTLCLFT |
| | | DFDSQINVPKTMESGTFITDKTVLDMKAMDSKSNGAIAW |
| | | SNQTSFTCQDIFKETNATYPSSDVPCDATLTEKSFETDM |
| | | NLNFQNLLVIVLRILLLKVAGFNLLMTLRLWSS |
| 650 | CDR1β | MNHEY |
| 651 | CDR2β | SMNVEV |
| 652 | CDR3β | ASSLLGGETQY |
| 653 | Vβ without signal | QVTQNPRYLITVTGKKLTVTCSQNMNHEYMSWYRQDPGL |
| | peptide (SignalP) | GLRQIYYSMNVEVTDKGDVPEGYKVSRKEKRNFPLILES |
| | | PSPNQTSLYFCASSLLGGETQYFGPGTRLLVL |
| 654 | Vβ (without the | MGPQLLGYVVLCLLGAGPLEAQVTQNPRYLITVTGKKLT |
| | Constant) | VTCSQNMNHEYMSWYRQDPGLGLRQIYYSMNVEVTDKGD |
| | | VPEGYKVSRKEKRNFPLILESPSPNQTSLYFCASSLLGG |
| | | ETQYFGPGTRLLVL |
| 655 | β chain with WT | MGPQLLGYVVLCLLGAGPLEAQVTQNPRYLITVTGKKLT |
| | signal peptide | VTCSQNMNHEYMSWYRQDPGLGLRQIYYSMNVEVTDKGD |
| | and constant Cβ | VPEGYKVSRKEKRNFPLILESPSPNQTSLYFCASSLLGG |
| | | ETQYFGPGTRLLVLEDLRNVTPPKVSLFEPSKAEIANKQ |
| | | KATLVCLARGFFPDHVELSWWVNGKEVHSGVSTDPQAYK |
| | | ESNYSYCLSSRLRVSATFWHNPRNHFRCQVQFHGLSEED |
| | | KWPEGSPKPVTQNISAEAWGRADCGITSASYQQGVLSAT |
| | | ILYEILLGKATLYAVLVSTLVVMAMVKRKNS |

In some embodiments, 0894-TCR92 interacts with and/or is specific for a peptide from gene KDMIA. In some embodiments, the peptide is from a neoantigen of KDM1A and has the amino acid change D691H (in which position 691 of the KDM1A protein is mutated from Asp to His). In some embodiments, 0894-TCR92 interacts with and/or is specific for the neoantigen in the context of HLA-DRA and DRB1*14:54.

TABLE 36

| SEQ ID | | |
|---|---|---|
| NO. | Description | 0894-TCR15 |
| 656 | CDR1α | YGATPY |
| 657 | CDR2α | YFSGDTLV |
| 658 | CDR3α | AADQTGANNLF |
| 659 | Vα without signal | QSVTQPDIHITVSEGASLELRCNYSYGATPYLFWYVQSP |
| | peptide (SignalP) | GQGLQLLLKYFSGDTLVQGIKGFEAEFKRSQSSFNLRKP |
| | | SVHWSDAAEYFCAADQTGANNLFFGTGTRLTVIP |
| 660 | Vα only (without | MLLELIPLLGIHFVLRTARAQSVTQPDIHITVSEGASLE |
| | the Constant) | LRCNYSYGATPYLFWYVQSPGQGLQLLLKYFSGDTLVQG |
| | | IKGFEAEFKRSQSSFNLRKPSVHWSDAAEYFCAADQTGA |
| | | NNLFFGTGTRLTVIP |
| 661 | α chain with WT | MLLELIPLLGIHFVLRTARAQSVTQPDIHITVSEGASLE |
| | signal peptide | LRCNYSYGATPYLFWYVQSPGQGLQLLLKYFSGDTLVQG |
| | and constant Cα | IKGFEAEFKRSQSSFNLRKPSVHWSDAAEYFCAADQTGA |
| | | NNLFFGTGTRLTVIPNIQNPEPAVYQLKDPRSQDSTLCL |
| | | FTDFDSQINVPKTMESGTFITDKTVLDMKAMDSKSNGAI |
| | | AWSNQTSFTCQDIFKETNATYPSSDVPCDATLTEKSFET |
| | | DMNLNFQNLLVIVLRILLLKVAGFNLLMTLRLWSS |
| 662 | CDR1β | MNHEY |
| 663 | CDR2β | SVGEGT |
| 664 | CDR3β | ASSPRGGYT |
| 665 | Vβ without signal | GVTQTPKFRVLKTGQSMTLLCAQDMNHEYMYWYRQDPGM |
| | peptide (SignalP) | GLRLIHYSVGEGTTAKGEVPDGYNVSRLKKQNFLLGLES |
| | | AAPSQTSVYFCASSPRGGYTFGSGTRLTVV |
| 666 | Vβ (without the | MSLGLLCCGAFSLLWAGPVNAGVTQTPKFRVLKTGQSMT |
| | Constant) | LLCAQDMNHEYMYWYRQDPGMGLRLIHYSVGEGTTAKGE |
| | | VPDGYNVSRLKKQNFLLGLESAAPSQTSVYFCASSPRGG |
| | | YTFGSGTRLTVV |
| 667 | β chain with WT | MSLGLLCCGAFSLLWAGPVNAGVTQTPKFRVLKTGQSMT |
| | signal peptide | LLCAQDMNHEYMYWYRQDPGMGLRLIHYSVGEGTTAKGE |
| | and constant Cβ | VPDGYNVSRLKKQNFLLGLESAAPSQTSVYFCASSPRGG |
| | | YTFGSGTRLTVVEDLRNVTPPKVSLFEPSKAEIANKQKA |
| | | TLVCLARGFFPDHVELSWWVNGKEVHSGVSTDPQAYKES |
| | | NYSYCLSSRLRVSATFWHNPRNHFRCQVQFHGLSEEDKW |
| | | PEGSPKPVTQNISAEAWGRADCGITSASYQQGVLSATIL |
| | | YEILLGKATLYAVLVSTLVVMAMVKRKNS |

In some embodiments, 0894-TCR15 interacts with and/or is specific for a peptide from gene USP9X. In some embodiments, the peptide is from a neoantigen of USP9X and has the amino acid change 11321M (in which position 1321 of the USP9X protein is mutated from Ile to Met). In some embodiments, 0894-TCR15 interacts with and/or is specific for the neoantigen in the context of DPA1*01:03 and DPB1*04:02.

TABLE 37

| SEQ ID | | |
|---|---|---|
| NO. | Description | 0894-TCR27 |
| 668 | CDR1α | VSNAYN |
| 669 | CDR2α | GSKP |
| 670 | CDR3α | AREAGTALI |
| 671 | Vα without signal | VAESKDQVFQPSTVASSEGAVVEIFCNHSVSNAYNFFWY |
| | peptide (SignalP) | LHFPGCAPRLLVKGSKPSQQGRYNMTYERFSSSLLILQV |
| | | READAAVYYCAREAGTALIFGKGTTLSVSS |
| 672 | Vα only (without | MALQSTLGAVWLGLLLNSLWKVAESKDQVFQPSTVASSE |
| | the Constant) | GAVVEIFCNHSVSNAYNFFWYLHFPGCAPRLLVKGSKPS |
| | | QQGRYNMTYERFSSSLLILQVREADAAVYYCAREAGTAL |
| | | IFGKGTTLSVSS |
| 673 | α chain with WT | MALQSTLGAVWLGLLLNSLWKVAESKDQVFQPSTVASSE |
| | signal peptide | GAVVEIFCNHSVSNAYNFFWYLHFPGCAPRLLVKGSKPS |
| | and constant Cα | QQGRYNMTYERFSSSLLILQVREADAAVYYCAREAGTAL |
| | | IFGKGTTLSVSSNIQNPEPAVYQLKDPRSQDSTLCLFTD |
| | | FDSQINVPKTMESGTFITDKTVLDMKAMDSKSNGAIAWS |
| | | NQTSFTCQDIFKETNATYPSSDVPCDATLTEKSFETDMN |
| | | LNFQNLLVIVLRILLLKVAGFNLLMTLRLWSS |
| 674 | CDR1β | MNHNS |
| 675 | CDR2β | SASEGT |
| 676 | CDR3β | ASRQDGSNQPQH |
| 677 | Vβ without signal | GVTQTPKFQVLKTGQSMTLQCAQDMNHNSMYWYRQDPGM |
| | peptide (SignalP) | GLRLIYYSASEGTTDKGEVPNGYNVSRLNKREFSLRLES |
| | | AAPSQTSVYFCASRQDGSNQPQHFGDGIRLSIL |
| 678 | Vβ (without the | MSIGLLCCVAFSLLWASPVNAGVTQTPKFQVLKTGQSMT |
| | Constant) | LQCAQDMNHNSMYWYRQDPGMGLRLIYYSASEGTTDKGE |
| | | VPNGYNVSRLNKREFSLRLESAAPSQTSVYFCASRQDGS |
| | | NQPQHFGDGIRLSIL |
| 679 | β chain with WT | MSIGLLCCVAFSLLWASPVNAGVTQTPKFQVLKTGQSMT |
| | signal peptide | LQCAQDMNHNSMYWYRQDPGMGLRLIYYSASEGTTDKGE |
| | and constant Cβ | VPNGYNVSRLNKREFSLRLESAAPSQTSVYFCASRQDGS |
| | | NQPQHFGDGIRLSILEDLRNVTPPKVSLFEPSKAEIANK |
| | | QKATLVCLARGFFPDHVELSWWVNGKEVHSGVSTDPQAY |
| | | KESNYSYCLSSRLRVSATFWHNPRNHFRCQVQFHGLSEE |
| | | DKWPEGSPKPVTQNISAEAWGRADCGITSASYQQGVLSA |
| | | TILYEILLGKATLYAVLVSTLVVMAMVKRKNS |

In some embodiments, 0894-TCR27 interacts with and/or is specific for a peptide from gene USP9X. In some embodiments, the peptide is from a neoantigen of USP9X and has the amino acid change 11321M (in which position 1321 of the USP9X protein is mutated from Ile to Met). In some embodiments, 0894-TCR27 interacts with and/or is specific 5 for the neoantigen in the context of DPA1*01:03 and DPB1*04:02.

TABLE 38

| SEQ ID | | |
|---|---|---|
| NO. | Description | 0894-TCR41 |
| 680 | CDR1α | YGATPY |
| 681 | CDR2α | YFSGDTLV |
| 682 | CDR3α | AVHSNDYKLS |
| 683 | Vα without signal | QSVTQPDIHITVSEGASLELRCNYSYGATPYLFWYVQSP |
| | peptide (SignalP) | GQGLQLLLKYFSGDTLVQGIKGFEAEFKRSQSSFNLRKP |
| | | SVHWSDAAEYFCAVHSNDYKLSFGAGTTVTVRA |
| 684 | Vα only (without | MLLELIPLLGIHFVLRTARAQSVTQPDIHITVSEGASLE |
| | the Constant) | LRCNYSYGATPYLFWYVQSPGQGLQLLLKYFSGDTLVQG |
| | | IKGFEAEFKRSQSSFNLRKPSVHWSDAAEYFCAVHSNDY |
| | | KLSFGAGTTVTVRA |
| 685 | α chain with WT | MLLELIPLLGIHFVLRTARAQSVTQPDIHITVSEGASLE |
| | signal peptide | LRCNYSYGATPYLFWYVQSPGQGLQLLLKYFSGDTLVQG |
| | and constant Cα | IKGFEAEFKRSQSSFNLRKPSVHWSDAAEYFCAVHSNDY |
| | | KLSFGAGTTVTVRANIQNPEPAVYQLKDPRSQDSTLCLF |
| | | TDFDSQINVPKTMESGTFITDKTVLDMKAMDSKSNGAIA |
| | | WSNQTSFTCQDFKETNATYPSSDVPCDATLTEKSFETD |
| | | MNLNFQNLLVIVLRILLLKVAGENLLMTLRLWSS |
| 686 | CDR1β | SGHTA |
| 687 | CDR2β | FQGNSA |
| 688 | CDR3β | ASSRRGTEAF |
| 689 | Vβ without signal | GVSQSPSNKVTEKGKDVELRCDPISGHTALYWYRQSLGQ |
| | peptide (SignalP) | GLEFLIYFQGNSAPDKSGLPSDRFSAERTGGSVSTLTIQ |
| | | RTQQEDSAVYLCASSRRGTEAFFGQGTRLTVV |
| 690 | Vβ (without the Constant) | MGTRLLFWVAFCLLGADHTGAGVSQSPSNKVTEKGKDVE |
| | | LRCDPISGHTALYWYRQSLGQGLEFLIYFQGNSAPDKSG |
| | | LPSDRFSAERTGGSVSTLTIQRTQQEDSAVYLCASSRRG |
| | | TEAFFGQGTRLTVV |
| 691 | β chain with WT | MGTRLLFWVAFCLLGADHTGAGVSQSPSNKVTEKGKDVE |
| | signal peptide | LRCDPISGHTALYWYRQSLGQGLEFLIYFQGNSAPDKSG |
| | and constant Cβ | LPSDRFSAERTGGSVSTLTIQRTQQEDSAVYLCASSRRG |
| | | TEAFFGQGTRLTVVEDLRNVTPPKVSLFEPSKAEIANKQ |
| | | KATLVCLARGFFPDHVELSWWVNGKEVHSGVSTDPQAYK |
| | | ESNYSYCLSSRLRVSATFWHNPRNHERCQVQFHGLSEED |
| | | KWPEGSPKPVTQNISAEAWGRADCGITSASYQQGVLSAT |
| | | ILYEILLGKATLYAVLVSTLVVMAMVKRKNS |

In some embodiments, 0894-TCR41 interacts with and/or is specific for a peptide from gene USP9X. In some embodiments, the peptide is from a neoantigen of USP9X and has the amino acid change 11321M (in which position 1321 of the USP9X protein is mutated from Ile to Met). In some embodiments, 0894-TCR41 interacts with and/or is specific for the neoantigen in the context of DPA1*01:03 and DPB1*04:02.

TABLE 39

| SEQ ID | | |
|---|---|---|
| NO. | Description | 0894-TCR78 |
| 692 | CDR1α | NIATNDY |
| 693 | CDR2α | GYKTK |
| 694 | CDR3α | LVGDIGYSGGGADGLT |
| 695 | Vα without signal | KTTQPISMDSYEGQEVNITCSHNNIATNDYITWYQQFPS |
| | peptide (SignalP) | QGPRFIIQGYKTKVTNEVASLFIPADRKSSTLSLPRVSL |
| | | SDTAVYYCLVGDIGYSGGGADGLTFGKGTHLIIQP |
| 696 | Vα only (without | MRQVARVIVELTLSTLSLAKTTQPISMDSYEGQEVNITC |
| | the Constant) | SHNNIATNDYITWYQQFPSQGPRFIIQGYKTKVTNEVAS |
| | | LFIPADRKSSTLSLPRVSLSDTAVYYCLVGDIGYSGGGA |
| | | DGLTFGKGTHLIIQP |
| 697 | α chain with WT | MRQVARVIVELTLSTLSLAKTTQPISMDSYEGQEVNITC |
| | signal peptide | SHNNIATNDYITWYQQFPSQGPRFIIQGYKTKVTNEVAS |
| | and constant Cα | LFIPADRKSSTLSLPRVSLSDTAVYYCLVGDIGYSGGGA |
| | | DGLTFGKGTHLIIQPNIQNPEPAVYQLKDPRSQDSTLCL |
| | | FTDFDSQINVPKTMESGTFITDKTVLDMKAMDSKSNGAI |
| | | AWSNQTSFTCQDIFKETNATYPSSDVPCDATLTEKSFET |
| | | DMNLNFQNLLVIVLRILLLKVAGFNLLMTLRLWSS |
| 698 | CDR1β | SNHLY |
| 699 | CDR2β | FYNNEI |
| 700 | CDR3β | ASSEQGAGDTQY |
| 701 | Vβ without signal | EPEVTQTPSHQVTQMGQEVILRCVPISNHLYFYWYRQIL |
| | peptide (SignalP) | GQKVEFLVSFYNNEISEKSEIFDDQFSVERPDGSNFTLK |
| | | IRSTKLEDSAMYFCASSEQGAGDTQYFGPGTRLTVL |
| 702 | Vβ (without the Constant) | MDTWLVCWAIFSLLKAGLTEPEVTQTPSHQVTQMGQEVI |
| | | LRCVPISNHLYFYWYRQILGQKVEFLVSFYNNEISEKSE |
| | | IFDDQFSVERPDGSNFTLKIRSTKLEDSAMYFCASSEQG |
| | | AGDTQYFGPGTRLTVL |
| 703 | β chain with WT | MDTWLVCWAIFSLLKAGLTEPEVTQTPSHQVTQMGQEVI |
| | signal peptide | LRCVPISNHLYFYWYRQILGQKVEFLVSFYNNEISEKSE |
| | and constant Cβ | IFDDQFSVERPDGSNFTLKIRSTKLEDSAMYFCASSEQG |
| | | AGDTQYFGPGTRLTVLEDLRNVTPPKVSLFEPSKAEIAN |
| | | KQKATLVCLARGFFPDHVELSWWVNGKEVHSGVSTDPQA |
| | | YKESNYSYCLSSRLRVSATFWHNPRNHFRCQVQFHGLSE |
| | | EDKWPEGSPKPVTQNISAEAWGRADCGITSASYQQGVLS |
| | | ATILYEILLGKATLYAVLVSTLVVMAMVKRKNS |

In some embodiments, 0894-TCR78 interacts with and/or is specific for a peptide from gene LLGL1. In some embodiments, the peptide is from a neoantigen of LLGL1 and has the amino acid change E966K (in which position 966 of the LLGL1 protein is mutated from Glu to Lys). In some embodiments, 0894-TCR78 interacts with and/or is specific for the

TABLE 40

| SEQ ID | | |
|---|---|---|
| NO. | Description | 0894-TCR8 |
| 704 | CDR1α | NSAFQY |
| 705 | CDR2α | TYSSGN |
| 706 | CDR3α | AMSEHYGGSQGNLI |
| 707 | Vα without signal | QQKEVEQDPGPLSVPEGAIVSLNCTYSNSAFQYEMWYRQ |
| | peptide (SignalP) | YSRKGPELLMYTYSSGNKEDGRETAQVDKSSKYISLFIR |
| | | DSQPSDSATYLCAMSEHYGGSQGNLIFGKGTKLSVKP |
| 708 | Vα only (without | MMKSLRVLLVILWLQLSWVWSQQKEVEQDPGPLSVPEGA |
| | the Constant) | IVSLNCTYSNSAFQYFMWYRQYSRKGPELLMYTYSSGNK |
| | | EDGRFTAQVDKSSKYISLFIRDSQPSDSATYLCAMSEHY |
| | | GGSQGNLIFGKGTKLSVKP |
| 709 | α chain with WT | MMKSLRVLLVILWLQLSWVWSQQKEVEQDPGPLSVPEGA |
| | signal peptide | IVSLNCTYSNSAFQYFMWYRQYSRKGPELLMYTYSSGNK |
| | and constant Cα | EDGRFTAQVDKSSKYISLFIRDSQPSDSATYLCAMSEHY |
| | | GGSQGNLIFGKGTKLSVKPNIQNPEPAVYQLKDPRSQDS |
| | | TLCLFTDFDSQINVPKTMESGTFITDKTVLDMKAMDSKS |
| | | NGAIAWSNQTSFTCQDIFKETNATYPSSDVPCDATLTEK |
| | | SFETDMNLNFQNLLVIVLRILLLKVAGFNLLMTLRLWSS |
| 710 | CDR1β | SGHDY |
| 711 | CDR2β | FNNNVP |
| 712 | CDR3β | ASSYGAGGPQH |
| 713 | Vβ without signal | GVIQSPRHEVTEMGQEVTLRCKPISGHDYLFWYRQTMMR |
| | peptide (SignalP) | GLELLIYFNNNVPIDDSGMPEDRESAKMPNASESTLKIQ |
| | | PSEPRDSAVYFCASSYGAGGPQHFGDGIRLSIL |
| 714 | Vβ (without the Constant) | MGSWTLCCVSLCILVAKHTDAGVIQSPRHEVTEMGQEVT |
| | | LRCKPISGHDYLFWYRQTMMRGLELLIYFNNNVPIDDSG |
| | | MPEDRFSAKMPNASFSTLKIQPSEPRDSAVYFCASSYGA |
| | | GGPQHFGDGIRLSIL |
| 715 | β chain with WT | MGSWTLCCVSLCILVAKHTDAGVIQSPRHEVTEMGQEVT |
| | signal peptide | LRCKPISGHDYLFWYRQTMMRGLELLIYFNNNVPIDDSG |
| | and constant Cβ | MPEDRFSAKMPNASESTLKIQPSEPRDSAVYFCASSYGA |
| | | GGPQHFGDGTRLSILEDLRNVTPPKVSLFEPSKAEIANK |
| | | QKATLVCLARGFFPDHVELSWWVNGKEVHSGVSTDPQAY |
| | | KESNYSYCLSSRLRVSATFWHNPRNHFRCQVQFHGLSEE |
| | | DKWPEGSPKPVTQNISAEAWGRADCGITSASYQQGVLSA |
| | | TILYEILLGKATLYAVLVSTLVVMAMVKRKNS |

In some embodiments, 0894-TCR8 interacts with and/or is specific for a peptide from gene ACO2. In some embodiments, the peptide is from a neoantigen of ACO2 and has the amino acid change H719Y (in which position 719 of the ACO2 protein is mutated from His to Tyr). In some embodiments, 0894-TCR8 interacts with and/or is specific for the neoantigen in the context of HLA-DRA and DRB1*14:54.

TABLE 41

| SEQ ID | | |
|---|---|---|
| NO. | Description | 0894-TCR20 |
| 716 | CDR1α | DSVNN |
| 717 | CDR2α | IPSGT |
| 718 | CDR3α | AVKSKSGGSNYKLT |
| 719 | Vα without signal peptide | IQVEQSPPDLILQEGANSTLRCNFSDSVNNLQWFHQNPW |
| | (SignalP) | GQLINLFYIPSGTKQNGRLSATTVATERYSLLYISSSQT |
| | | TDSGVYFCAVKSKSGGSNYKLTFGKGTLLTVNP |
| 720 | Vα only (without | MKRILGALLGLLSAQVCCVRGIQVEQSPPDLILQEGANS |
| | the Constant) | TLRCNFSDSVNNLQWFHQNPWGQLINLFYIPSGTKQNGR |
| | | LSATTVATERYSLLYISSSQTTDSGVYFCAVKSKSGGSN |
| | | YKLTFGKGTLLTVNP |
| 721 | α chain with WT | MKRILGALLGLLSAQVCCVRGIQVEQSPPDLILQEGANS |
| | signal peptide | TLRCNFSDSVNNLQWFHQNPWGQLINLFYIPSGTKQNGR |
| | and constant Cα | LSATTVATERYSLLYISSSQTTDSGVYFCAVKSKSGGSN |
| | | YKLTFGKGTLLTVNPNIQNPEPAVYQLKDPRSQDSTLCL |
| | | FTDFDSQINVPKTMESGTFITDKTVLDMKAMDSKSNGAI |
| | | AWSNQTSFTCQDIFKETNATYPSSDVPCDATLTEKSFET |
| | | DMNLNFQNLLVIVLRILLLKVAGFNLLMTLRLWSS |
| 722 | CDR1β | SNHLY |
| 723 | CDR2β | FYNNEI |
| 724 | CDR3β | ASSATGYAF |
| 725 | Vβ without signal | EPEVTQTPSHQVTQMGQEVILRCVPISNHLYFYWYRQIL |
| | peptide (SignalP) | GQKVEFLVSFYNNEISEKSEIFDDQFSVERPDGSNFTLK |
| | | IRSTKLEDSAMYFCASSATGYAFFGQGTRLTVV |
| 726 | Vβ (without the Constant) | MDTWLVCWAIFSLLKAGLTEPEVTQTPSHQVTQMGQEVI |
| | | LRCVPISNHLYFYWYRQILGQKVEFLVSFYNNEISEKSE |
| | | IFDDQFSVERPDGSNFTLKIRSTKLEDSAMYFCASSATG |
| | | YAFFGQGTRLTVV |
| 727 | β chain with WT | MDTWLVCWAIFSLLKAGLTEPEVTQTPSHQVTQMGQEVI |
| | signal peptide | LRCVPISNHLYFYWYRQILGQKVEFLVSFYNNEISEKSE |
| | and constant Cβ | IFDDQFSVERPDGSNFTLKIRSTKLEDSAMYFCASSATG |
| | | YAFFGQGTRLTVVEDLRNVTPPKVSLFEPSKAEIANKQK |
| | | ATLVCLARGFFPDHVELSWWVNGKEVHSGVSTDPQAYKE |
| | | SNYSYCLSSRLRVSATFWHNPRNHFRCQVQFHGLSEEDK |
| | | WPEGSPKPVTQNISAEAWGRADCGITSASYQQGVLSATI |
| | | LYEILLGKATLYAVLVSTLVVMAMVKRKNS |

In some embodiments, 0894-TCR20 interacts with and/or is specific for a peptide from gene ACO2. In some embodiments, the peptide is from a neoantigen of ACO2 and has the amino acid change H719Y (in which position 719 of the ACO2 protein is mutated from His to Tyr). In some embodiments, 0894-TCR20 interacts with and/or is specific for the

TABLE 42

| SEQ ID | | |
|---|---|---|
| NO. | Description | 0894-TCR22-2 |
| 728 | CDR1α | DSVNN |
| 729 | CDR2α | IPSGT |
| 730 | CDR3α | AVDGYGGSQGNLI |
| 731 | Vα without signal peptide | IQVEQSPPDLILQEGANSTLRCNFSDSVNNLQWFHQNPW |
| | (SignalP) | GQLINLFYIPSGTKQNGRLSATTVATERYSLLYISSSQT |
| | | TDSGVYFCAVDGYGGSQGNLIFGKGTKLSVKP |
| 732 | Vα only (without | MKRILGALLGLLSAQVCCVRGIQVEQSPPDLILQEGANS |
| | the Constant) | TLRCNFSDSVNNLQWFHQNPWGQLINLFYIPSGTKQNGR |
| | | LSATTVATERYSLLYISSSQTTDSGVYFCAVDGYGGSQG |
| | | NLIFGKGTKLSVKP |
| 733 | α chain with WT | MKRILGALLGLLSAQVCCVRGIQVEQSPPDLILQEGANS |
| | signal peptide | TLRCNFSDSVNNLQWFHQNPWGQLINLFYIPSGTKQNGR |
| | and constant Cα | LSATTVATERYSLLYISSSQTTDSGVYFCAVDGYGGSQG |
| | | NLIFGKGTKLSVKPNIQNPEPAVYQLKDPRSQDSTLCLF |
| | | TDFDSQINVPKTMESGTFITDKTVLDMKAMDSKSNGAIA |
| | | WSNQTSFTCQDIFKETNATYPSSDVPCDATLTEKSFETD |
| | | MNLNFQNLLVIVLRILLLKVAGFNLLMTLRLWSS |
| 734 | CDR1β | SNHLY |
| 735 | CDR2β | FYNNEI |
| 736 | CDR3β | ASRGDTEAF |
| 737 | Vβ without signal | EPEVTQTPSHQVTQMGQEVILRCVPISNHLYFYWYRQIL |
| | peptide (SignalP) | GQKVEFLVSFYNNEISEKSEIFDDQFSVERPDGSNFTLK |
| | | IRSTKLEDSAMYFCASRGDTEAFFGQGTRLTVV |
| 738 | Vβ (without the Constant) | MDTWLVCWAIFSLLKAGLTEPEVTQTPSHQVTQMGQEVI |
| | | LRCVPISNHLYFYWYRQILGQKVEFLVSFYNNEISEKSE |
| | | IFDDQFSVERPDGSNFTLKIRSTKLEDSAMYFCASRGDT |
| | | EAFFGQGTRLTVV |
| 739 | β chain with WT | MDTWLVCWAIFSLLKAGLTEPEVTQTPSHQVTQMGQEVI |
| | signal peptide | LRCVPISNHLYFYWYRQILGQKVEFLVSFYNNEISEKSE |
| | and constant Cβ | IFDDQFSVERPDGSNFTLKIRSTKLEDSAMYFCASRGDT |
| | | EAFFGQGTRLTVVEDLRNVTPPKVSLFEPSKAEIANKQK |
| | | ATLVCLARGFFPDHVELSWWVNGKEVHSGVSTDPQAYKE |
| | | SNYSYCLSSRLRVSATFWHNPRNHFRCQVQFHGLSEEDK |
| | | WPEGSPKPVTQNISAEAWGRADCGITSASYQQGVLSATI |
| | | LYEILLGKATLYAVLVSTLVVMAMVKRKNS |

In some embodiments, 0894-TCR22-2 interacts with and/or is specific for a peptide from gene ACO2. In some embodiments, the peptide is from a neoantigen of ACO2 and has the amino acid change H719Y (in which position 719 of the ACO2 protein is mutated from His to Tyr). In some embodiments, 0894-TCR22-2 interacts with and/or is specific for the neoantigen in the context of HLA-DRA and DRB1*14:54.

TABLE 43

| SEQ ID | | |
|---|---|---|
| NO. | Description | 0894-TCR29 |
| 740 | CDR1α | DSVNN |
| 741 | CDR2α | IPSGT |
| 742 | CDR3α | AVDRKSGGSNYKLT |
| 743 | Vα without signal peptide | IQVEQSPPDLILQEGANSTLRCNFSDSVNNLQWFHQNPW |
| | (SignalP) | GQLINLFYIPSGTKQNGRLSATTVATERYSLLYISSSQT |
| | | TDSGVYFCAVDRKSGGSNYKLTFGKGTLLTVNP |
| 744 | Vα only (without | MKRILGALLGLLSAQVCCVRGIQVEQSPPDLILQEGANS |
| | the Constant) | TLRCNFSDSVNNLQWFHQNPWGQLINLFYIPSGTKQNGR |
| | | LSATTVATERYSLLYISSSQTTDSGVYFCAVDRKSGGSN |
| | | YKLTFGKGTLLTVNP |
| 745 | α chain with WT | MKRILGALLGLLSAQVCCVRGIQVEQSPPDLILQEGANS |
| | signal peptide | TLRCNFSDSVNNLQWFHQNPWGQLINLFYIPSGTKQNGR |
| | and constant Cα | LSATTVATERYSLLYISSSQTTDSGVYFCAVDRKSGGSN |
| | | YKLTFGKGTLLTVNPNIQNPEPAVYQLKDPRSQDSTLCL |
| | | FTDFDSQINVPKTMESGTFITDKTVLDMKAMDSKSNGAI |
| | | AWSNQTSFTCQDIFKETNATYPSSDVPCDATLTEKSFET |
| | | DMNLNFQNLLVIVLRILLLKVAGFNLLMTLRLWSS |
| 746 | CDR1β | SGHNS |
| 747 | CDR2β | FNNNVP |
| 748 | CDR3β | ASSLSSEAF |
| 749 | Vβ without signal | GVIQSPRHEVTEMGQEVTLRCKPISGHNSLFWYRQTMMR |
| | peptide (SignalP) | GLELLIYFNNNVPIDDSGMPEDRFSAKMPNASFSTLKIQ |
| | | PSEPRDSAVYFCASSLSSEAFFGQGTRLTVV |
| 750 | Vβ (without the Constant) | MDSWTFCCVSLCILVAKHTDAGVIQSPRHEVTEMGQEVT |
| | | LRCKPISGHNSLFWYRQTMMRGLELLIYFNNNVPIDDSG |
| | | MPEDRFSAKMPNASFSTLKIQPSEPRDSAVYFCASSLSS |
| | | EAFFGQGTRLTVV |
| 751 | β chain with WT | MDSWTFCCVSLCILVAKHTDAGVIQSPRHEVTEMGQEVT |
| | signal peptide | LRCKPISGHNSLFWYRQTMMRGLELLIYFNNNVPIDDSG |
| | and constant Cβ | MPEDRFSAKMPNASFSTLKIQPSEPRDSAVYFCASSLSS |
| | | EAFFGQGTRLTVVEDLRNVTPPKVSLFEPSKAEIANKQK |
| | | ATLVCLARGFFPDHVELSWWVNGKEVHSGVSTDPQAYKE |
| | | SNYSYCLSSRLRVSATFWHNPRNHFRCQVQFHGLSEEDK |
| | | WPEGSPKPVTQNISAEAWGRADCGITSASYQQGVLSATI |
| | | LYEILLGKATLYAVLVSTLVVMAMVKRKNS |

In some embodiments, 0894-TCR29 interacts with and/or is specific for a peptide from gene ACO2. In some embodiments, the peptide is from a neoantigen of ACO2 and has the amino acid change H719Y (in which position 719 of the ACO2 protein is mutated from His to Tyr). In some embodiments, 0894-TCR29 interacts with and/or is specific for the

TABLE 44

| SEQ ID | | |
|---|---|---|
| NO. | Description | 0894-TCR31-1 |
| 752 | CDR1α | TSESNYY |
| 753 | CDR2α | QEAYKQQN |
| 754 | CDR3α | AFMKPHPAGGTSYGKLT |
| 755 | Vα without signal | QTVTQSQPEMSVQEAETVTLSCTYDTSESNYYLFWYKQP |
| | peptide (SignalP) | PSRQMILVIRQEAYKQQNATENRFSVNFQKAAKSFSLKI |
| | | SDSQLGDTAMYFCAFMKPHPAGGTSYGKLTFGQGTILTV |
| | | HP |
| 756 | Vα only (without | MTRVSLLWAVVVSTCLESGMAQTVTQSQPEMSVQEAETV |
| | the Constant) | TLSCTYDTSESNYYLFWYKQPPSRQMILVIRQEAYKQQN |
| | | ATENRFSVNFQKAAKSFSLKISDSQLGDTAMYFCAFMKP |
| | | HPAGGTSYGKLTFGQGTILTVHP |
| 757 | α chain with WT signal | MTRVSLLWAVVVSTCLESGMAQTVTQSQPEMSVQEAETV |
| | peptide and constant Cα | TLSCTYDTSESNYYLFWYKQPPSRQMILVIRQEAYKQQN |
| | | ATENRFSVNFQKAAKSFSLKISDSQLGDTAMYFCAFMKP |
| | | HPAGGTSYGKLTFGQGTILTVHP |
| 758 | CDR1β | MNHEY |
| 759 | CDR2β | SVGAGI |
| 760 | CDR3β | ASRVGRSVGTGELF |
| 761 | Vβ without signal | GVTQTPKFQVLKTGQSMTLQCAQDMNHEYMSWYRQDPGM |
| | peptide (SignalP) | GLRLIHYSVGAGITDQGEVPNGYNVSRSTTEDFPLRLLS |
| | | AAPSQTSVYFCASRVGRSVGTGELFFGEGSRLTVL |
| 762 | Vβ (without the Constant) | MSIGLLCCAALSLLWAGPVNAGVTQTPKFQVLKTGQSMT |
| | | LQCAQDMNHEYMSWYRQDPGMGLRLIHYSVGAGITDQGE |
| | | VPNGYNVSRSTTEDFPLRLLSAAPSQTSVYFCASRVGRS |
| | | VGTGELFFGEGSRLTVL |
| 763 | β chain with WT | MSIGLLCCAALSLLWAGPVNAGVTQTPKFQVLKTGQSMT |
| | signal peptide | LQCAQDMNHEYMSWYRQDPGMGLRLIHYSVGAGITDQGE |
| | and constant Cβ | VPNGYNVSRSTTEDFPLRLLSAAPSQTSVYFCASRVGRS |
| | | VGTGELFFGEGSRLTVLEDLRNVTPPKVSLFEPSKAEIA |
| | | NKQKATLVCLARGFFPDHVELSWWVNGKEVHSGVSTDPQ |
| | | AYKESNYSYCLSSRLRVSATFWHNPRNHFRCQVQFHGLS |
| | | EEDKWPEGSPKPVTQNISAEAWGRADCGITSASYQQGVL |
| | | SATILYEILLGKATLYAVLVSTLVVMAMVKRKNS |

In some embodiments, 0894-TCR31-1 interacts with and/or is specific for a peptide from gene ACO2. In some embodiments, the peptide is from a neoantigen of ACO2 and has the amino acid change H719Y (in which position 719 of the ACO2 protein is mutated from His to Tyr). In some embodiments, 0894-TCR31-1 interacts with and/or is specific for 5 the neoantigen in the context of HLA-DRA and DRB1*14:54.

TABLE 45

| SEQ ID | | |
|---|---|---|
| NO. | Description | 0894-TCR36 |
| 764 | CDR1α | TSESNYY |
| 765 | CDR2α | QEAYKQQN |
| 766 | CDR3α | AFMTPNNNNDMR |
| 767 | Vα without signal | QTVTQSQPEMSVQEAETVTLSCTYDTSESNYYLFWYKQP |
| | peptide (SignalP) | PSRQMILVIRQEAYKQQNATENRFSVNFQKAAKSFSLKI |
| | | SDSQLGDTAMYFCAFMTPNNNNDMRFGAGTRLTVKP |
| 768 | Vα only (without | MTRVSLLWAVVVSTCLESGMAQTVTQSQPEMSVQEAETV |
| | the Constant) | TLSCTYDTSESNYYLFWYKQPPSRQMILVIRQEAYKQQN |
| | | ATENRFSVNFQKAAKSFSLKISDSQLGDTAMYFCAFMTP |
| | | NNNNDMRFGAGTRLTVKP |
| 769 | α chain with WT | MTRVSLLWAVVVSTCLESGMAQTVTQSQPEMSVQEAETV |
| | signal peptide | TLSCTYDTSESNYYLFWYKQPPSRQMILVIRQEAYKQQN |
| | and constant Cα | ATENRFSVNFQKAAKSFSLKISDSQLGDTAMYFCAFMTP |
| | | NNNNDMRFGAGTRLTVKPNIQNPEPAVYQLKDPRSQDST |
| | | LCLFTDFDSQINVPKTMESGTFITDKTVLDMKAMDSKSN |
| | | GAIAWSNQTSFTCQDIFKETNATYPSSDVPCDATLTEKS |
| | | FETDMNLNFQNLLVIVLRILLLKVAGFNLLMTLRLWSS |
| 770 | CDR1β | KGHSH |
| 771 | CDR2β | LQKENI |
| 772 | CDR3β | ASSPGSYSPLH |
| 773 | Vβ without signal | GVMQNPRHLVRRRGQEARLRCSPMKGHSHVYWYRQLPEE |
| | peptide (SignalP) | GLKFMVYLQKENIIDESGMPKERFSAEFPKEGPSILRIQ |
| | | QVVRGDSAAYFCASSPGSYSPLHFGNGTRLTVT |
| 774 | Vβ (without the Constant) | MDTRLLCCAVICLLGAGLSNAGVMQNPRHLVRRRGQEAR |
| | | LRCSPMKGHSHVYWYRQLPEEGLKEMVYLQKENIIDESG |
| | | MPKERFSAEFPKEGPSILRIQQVVRGDSAAYFCASSPGS |
| | | YSPLHFGNGTRLTVT |
| 775 | β chain with WT | MDTRLLCCAVICLLGAGLSNAGVMQNPRHLVRRRGQEAR |
| | signal peptide | LRCSPMKGHSHVYWYRQLPEEGLKEMVYLQKENIIDESG |
| | and constant Cβ | MPKERFSAEFPKEGPSILRIQQVVRGDSAAYFCASSPGS |
| | | YSPLHFGNGTRLTVTEDLRNVTPPKVSLFEPSKAEIANK |
| | | QKATLVCLARGFFPDHVELSWWVNGKEVHSGVSTDPQAY |
| | | KESNYSYCLSSRLRVSATFWHNPRNHFRCQVQFHGLSEE |
| | | DKWPEGSPKPVTQNISAEAWGRADCGITSASYQQGVLSA |
| | | TILYEILLGKATLYAVLVSTLVVMAMVKRKNS |

In some embodiments, 0894-TCR36 interacts with and/or is specific for a peptide from gene ACO2. In some embodiments, the peptide is from a neoantigen of ACO2 and has the amino acid change H719Y (in which position 719 of the ACO2 protein is mutated from His to Tyr). In some embodiments, 0894-TCR36 interacts with and/or is specific for the

TABLE 46

| SEQ ID | | |
|---|---|---|
| NO. | Description | 0894-TCR13 |
| 776 | CDR1α | NSASQS |
| 777 | CDR2α | VYSSGN |
| 778 | CDR3α | VVNSGGGSQGNLI |
| 779 | Vα without signal | QRKEVEQDPGPFNVPEGATVAFNCTYSNSASQSFFWYRQ |
| | peptide (SignalP) | DCRKEPKLLMSVYSSGNEDGRFTAQLNRASQYISLLIRD |
| | | SKLSDSATYLCVVNSGGGSQGNLIFGKGTKLSVKP |
| 780 | Vα only (without | MISLRVLLVILWLQLSWVWSQRKEVEQDPGPFNVPEGAT |
| | the Constant) | VAFNCTYSNSASQSFFWYRQDCRKEPKLLMSVYSSGNED |
| | | GRFTAQLNRASQYISLLIRDSKLSDSATYLCVVNSGGGS |
| | | QGNLIFGKGTKLSVKP |
| 781 | α chain with WT | MISLRVLLVILWLQLSWVWSQRKEVEQDPGPFNVPEGAT |
| | signal peptide | VAFNCTYSNSASQSFFWYRQDCRKEPKLLMSVYSSGNED |
| | and constant Cα | GRFTAQLNRASQYISLLIRDSKLSDSATYLCVVNSGGGS |
| | | QGNLIFGKGTKLSVKPNIQNPEPAVYQLKDPRSQDSTLC |
| | | LFTDFDSQINVPKTMESGTFITDKTVLDMKAMDSKSNGA |
| | | IAWSNQTSFTCQDIFKETNATYPSSDVPCDATLTEKSFE |
| | | TDMNLNFQNLLVIVLRILLLKVAGFNLLMTLRLWSS |
| 782 | CDR1β | LNHDA |
| 783 | CDR2β | SQIVND |
| 784 | CDR3β | ASSILTGNNSPLH |
| 785 | Vβ without signal | GITQSPKYLFRKEGQNVTLSCEQNLNHDAMYWYRQDPGQ |
| | peptide (SignalP) | GLRLIYYSQIVNDFQKGDIAEGYSVSREKKESFPLTVTS |
| | | AQKNPTAFYLCASSILTGNNSPLHFGNGTRLTVT |
| 786 | Vβ (without the Constant) | MSNQVLCCVVLCLLGANTVDGGITQSPKYLFRKEGQNVT |
| | | LSCEQNLNHDAMYWYRQDPGQGLRLIYYSQIVNDFQKGD |
| | | IAEGYSVSREKKESFPLTVTSAQKNPTAFYLCASSILTG |
| | | NNSPLHFGNGTRLTVT |
| 787 | β chain with WT | MSNQVLCCVVLCLLGANTVDGGITQSPKYLFRKEGQNVT |
| | signal peptide | LSCEQNLNHDAMYWYRQDPGQGLRLIYYSQIVNDFQKGD |
| | and constant Cβ | IAEGYSVSREKKESFPLTVTSAQKNPTAFYLCASSILTG |
| | | NNSPLHFGNGTRLTVTEDLRNVTPPKVSLFEPSKAEIAN |
| | | KQKATLVCLARGFFPDHVELSWWVNGKEVHSGVSTDPQA |
| | | YKESNYSYCLSSRLRVSATFWHNPRNHFRCQVQFHGLSE |
| | | EDKWPEGSPKPVTQNISAEAWGRADCGITSASYQQGVLS |
| | | ATILYEILLGKATLYAVLVSTLVVMAMVKRKNS |

In some embodiments, 0894-TCR13 interacts with and/or is specific for a peptide from gene POLDIP3. In some embodiments, the peptide is from a neoantigen of POLDIP3 and has the amino acid change S400F (in which position 400 of the POLDIP3 protein is mutated from Ser to Phe). In some embodiments, 0894-TCR13 interacts with and/or is specific for the neoantigen in the context of DPA1*01:03 and DPB1*03:01.

TABLE 47

| SEQ ID | | |
|---|---|---|
| NO. | Description | 0894-TCR44 |
| 788 | CDR1α | NSAFQY |
| 789 | CDR2α | TYSSGN |
| 790 | CDR3α | AMSRSDTGNQFY |
| 791 | Vα without signal | QQKEVEQDPGPLSVPEGAIVSLNCTYSNSAFQYFMWYRQ |
| | peptide (SignalP) | YSRKGPELLMYTYSSGNKEDGRFTAQVDKSSKYISLFIR |
| | | DSQPSDSATYLCAMSRSDTGNQFYFGTGTSLTVIP |
| 792 | Vα only (without | MMKSLRVLLVILWLQLSWVWSQQKEVEQDPGPLSVPEGA |
| | the Constant) | IVSLNCTYSNSAFQYFMWYRQYSRKGPELLMYTYSSGNK |
| | | EDGRFTAQVDKSSKYISLFIRDSQPSDSATYLCAMSRSD |
| | | TGNQFYFGTGTSLTVIP |
| 793 | α chain with WT | MMKSLRVLLVILWLQLSWVWSQQKEVEQDPGPLSVPEGA |
| | signal peptide | IVSLNCTYSNSAFQYFMWYRQYSRKGPELLMYTYSSGNK |
| | and constant Cα | EDGRFTAQVDKSSKYISLFIRDSQPSDSATYLCAMSRSD |
| | | TGNQFYFGTGTSLTVIPNIQNPEPAVYQLKDPRSQDSTL |
| | | CLFTDFDSQINVPKTMESGTFITDKTVLDMKAMDSKSNG |
| | | AIAWSNQTSFTCQDIFKETNATYPSSDVPCDATLTEKSF |
| | | ETDMNLNFQNLLVIVLRILLLKVAGFNLLMTLRLWSS |
| 794 | CDR1β | SGHAT |
| 795 | CDR2β | FQNNGV |
| 796 | CDR3β | ASSFGPGVTDTQY |
| 797 | Vβ without signal | GVAQSPRYKIIEKRQSVAFWCNPISGHATLYWYQQILGQ |
| | peptide (SignalP) | GPKLLIQFQNNGVVDDSQLPKDRFSAERLKGVDSTLKIQ |
| | | PAKLEDSAVYLCASSFGPGVTDTQYFGPGTRLTVL |
| 798 | Vβ (without the Constant) | MGTRLLCWAALCLLGAELTEAGVAQSPRYKIIEKRQSVA |
| | | FWCNPISGHATLYWYQQILGQGPKLLIQFQNNGVVDDSQ |
| | | LPKDRFSAERLKGVDSTLKIQPAKLEDSAVYLCASSFGP |
| | | GVTDTQYFGPGTRLTVL |
| 799 | β chain with WT | MGTRLLCWAALCLLGAELTEAGVAQSPRYKIIEKRQSVA |
| | signal peptide | FWCNPISGHATLYWYQQILGQGPKLLIQFQNNGVVDDSQ |
| | and constant Cβ | LPKDRFSAERLKGVDSTLKIQPAKLEDSAVYLCASSFGP |
| | | GVTDTQYFGPGTRLTVLEDLRNVTPPKVSLFEPSKAEIA |
| | | NKQKATLVCLARGFFPDHVELSWWVNGKEVHSGVSTDPQ |
| | | AYKESNYSYCLSSRLRVSATFWHNPRNHFRCQVQFHGLS |
| | | EEDKWPEGSPKPVTQNISAEAWGRADCGITSASYQQGVL |
| | | SATILYEILLGKATLYAVLVSTLVVMAMVKRKNS |

In some embodiments, 0894-TCR44 interacts with and/or is specific for a peptide from gene POLDIP3. In some embodiments, the peptide is from a neoantigen of POLDIP3 and has the amino acid change S400F (in which position 400 of the POLDIP3 protein is mutated from Ser to Phe). In some embodiments, 0894-TCR44 interacts with and/or is specific for the neoantigen in the context of DPA1*01:03 and DPB1*03:01.

TABLE 48

| SEQ ID | | |
|---|---|---|
| NO. | Description | 5040-TCR1 |
| 800 | CDR1α | VSGNPY |
| 801 | CDR2α | YITGDNLV |
| 802 | CDR3α | AVRDYFGNTGKLI |
| 803 | Vα without signal | QSVAQPEDQVNVAEGNPLTVKCTYSVSGNPYLFWYVQYP |
| | peptide (SignalP) | NRGLQFLLKYITGDNLVKGSYGFEAEFNKSQTSFHLKKP |
| | | SALVSDSALYFCAVRDYFGNTGKLIFGQGTTLQVKPNIQ |
| | | NPEPAV |
| 804 | Vα only (without | MASAPISMLAMLFTLSGLRAQSVAQPEDQVNVAEGNPLT |
| | the Constant) | VKCTYSVSGNPYLFWYVQYPNRGLQFLLKYITGDNLVKG |
| | | SYGFEAEFNKSQTSFHLKKPSALVSDSALYFCAVRDYFG |
| | | NTGKLIFGQGTTLQVKPNIQNPEPAV |
| 805 | α chain with WT | MASAPISMLAMLFTLSGLRAQSVAQPEDQVNVAEGNPLT |
| | signal peptide | VKCTYSVSGNPYLFWYVQYPNRGLQFLLKYITGDNLVKG |
| | and constant Cα | SYGFEAEFNKSQTSFHLKKPSALVSDSALYFCAVRDYFG |
| | | NTGKLIFGQGTTLQVKPNIQNPEPAVNIQNPEPAVYQLK |
| | | DPRSQDSTLCLFTDFDSQINVPKTMESGTFITDKTVLDM |
| | | KAMDSKSNGAIAWSNQTSFTCQDIFKETNATYPSSDVPC |
| | | DATLTEKSFETDMNLNFQNLLVIVLRILLLKVAGFNLLM |
| | | TLRLWSS |
| 806 | CDR1β | SQVTM |
| 807 | CDR2β | ANQGSEA |
| 808 | CDR3β | SVALTENTEAF |
| 809 | Vβ without signal | AVISQKPSRDICQRGTSLTIQCQVDSQVTMMFWYRQQPG |
| | peptide (SignalP) | QSLTLIATANQGSEATYESGFVIDKFPISRPNLTFSTLT |
| | | VSNMSPEDSSIYLCSVALTENTEAFFGQGTRLTVVEDLR |
| | | N |
| 810 | Vβ (without the Constant) | MLSLLLLLLGLGSVFSAVISQKPSRDICQRGTSLTIQCQ |
| | | VDSQVTMMFWYRQQPGQSLTLIATANQGSEATYESGFVI |
| | | DKFPISRPNLTFSTLTVSNMSPEDSSIYLCSVALTENTE |
| | | AFFGQGTRLTVVEDLRN |
| 811 | β chain with WT | MLSLLLLLLGLGSVESAVISQKPSRDICQRGTSLTIQCQ |
| | signal peptide | VDSQVTMMFWYRQQPGQSLTLIATANQGSEATYESGFVI |
| | and constant Cβ | DKFPISRPNLTFSTLTVSNMSPEDSSIYLCSVALTENTE |
| | | AFFGQGTRLTVVEDLRNEDLRNVTPPKVSLFEPSKAEIA |
| | | NKQKATLVCLARGFFPDHVELSWWVNGKEVHSGVSTDPQ |
| | | AYKESNYSYCLSSRLRVSATFWHNPRNHFRCQVQFHGLS |
| | | EEDKWPEGSPKPVTQNISAEAWGRADCGITSASYQQGVL |
| | | SATILYEILLGKATLYAVLVSTLVVMAMVKRKNS |

In some embodiments, 5040-TCR1 interacts with and/or is specific for a peptide from gene EMC8. In some embodiments, the peptide is from a neoantigen of EMC8 and has the amino acid change T140M (in which position 140 of the EMC8 protein is mutated from Thr to Met). In some embodiments, 5040-TCR1 interacts with and/or is specific for the neoantigen in the context of HLA-B*15:01.

TABLE 49

| SEQ ID | | |
|---|---|---|
| NO. | Description | 5040-TCR40 |
| 812 | CDR1α | TSGFNG |
| 813 | CDR2α | NVLDGL |
| 814 | CDR3α | AVRGDSWGKLQ |
| 815 | Vα without signal | QNIDQPTEMTATEGAIVQINCTYQTSGFNGLFWYQQHAG |
| | peptide (SignalP) | EAPTFLSYNVLDGLEEKGRFSSFLSRSKGYSYLLLKELQ |
| | | MKDSASYLCAVRGDSWGKLQFGAGTQVVVTPNIQNPEPA |
| | | V |
| 816 | Vα only (without | MWGVFLLYVSMKMGGTTGQNIDQPTEMTATEGAIVQINC |
| | the Constant) | TYQTSGFNGLFWYQQHAGEAPTFLSYNVLDGLEEKGRFS |
| | | SFLSRSKGYSYLLLKELQMKDSASYLCAVRGDSWGKLQF |
| | | GAGTQVVVTPNIQNPEPAV |
| 817 | α chain with WT | MWGVFLLYVSMKMGGTTGQNIDQPTEMTATEGAIVQINC |
| | signal peptide | TYQTSGFNGLFWYQQHAGEAPTFLSYNVLDGLEEKGRFS |
| | and constant Cα | SFLSRSKGYSYLLLKELQMKDSASYLCAVRGDSWGKLQF |
| | | GAGTQVVVTPNIQNPEPAVNIQNPEPAVYQLKDPRSQDS |
| | | TLCLFTDFDSQINVPKTMESGTFITDKTVLDMKAMDSKS |
| | | NGAIAWSNQTSFTCQDIFKETNATYPSSDVPCDATLTEK |
| | | SFETDMNLNFQNLLVIVLRILLLKVAGFNLLMTLRLWSS |
| 818 | CDR1β | SGHTA |
| 819 | CDR2β | FQGNSA |
| 820 | CDR3β | ASSPAAGDEHEQY |
| 821 | Vβ without signal | GVSQSPSNKVTEKGKDVELRCDPISGHTALYWYRQSLGQ |
| | peptide (SignalP) | GLEFLIYFQGNSAPDKSGLPSDRFSAERTGGSVSTLTIQ |
| | | RTQQEDSAVYLCASSPAAGDEHEQYFGPGTRLTVTEDLR |
| | | N |
| 822 | Vβ (without the | MGTRLLFWVAFCLLGADHTGAGVSQSPSNKVTEKGKDVE |
| | Constant) | LRCDPISGHTALYWYRQSLGQGLEFLIYFQGNSAPDKSG |
| | | LPSDRFSAERTGGSVSTLTIQRTQQEDSAVYLCASSPAA |
| | | GDEHEQYFGPGTRLTVTEDLRN |
| 823 | β chain with WT | MGTRLLFWVAFCLLGADHTGAGVSQSPSNKVTEKGKDVE |
| | signal peptide | LRCDPISGHTALYWYRQSLGQGLEFLIYFQGNSAPDKSG |
| | and constant Cβ | LPSDRFSAERTGGSVSTLTIQRTQQEDSAVYLCASSPAA |
| | | GDEHEQYFGPGTRLTVTEDLRNEDLRNVTPPKVSLFEPS |
| | | KAEIANKQKATLVCLARGFFPDHVELSWWVNGKEVHSGV |
| | | STDPQAYKESNYSYCLSSRLRVSATFWHNPRNHFRCQVQ |
| | | FHGLSEEDKWPEGSPKPVTQNISAEAWGRADCGITSASY |
| | | QQGVLSATILYEILLGKATLYAVLVSTLVVMAMVKRKNS |

In some embodiments, 5040-TCR40 interacts with and/or is specific for a peptide from gene EMC8. In some embodiments, the peptide is from a neoantigen of EMC8 and has the amino acid change T140M (in which position 140 of the EMC8 protein is mutated from Thr to Met). In some embodiments, 5040-TCR40 interacts with and/or is specific for the neoantigen in the context of HLA-B*15:01.

TABLE 50

| SEQ ID | | |
|---|---|---|
| NO. | Description | 5040-TCR45 |
| 824 | CDR1α | NSASDY |
| 825 | CDR2α | IRSNMDK |
| 826 | CDR3α | AENPGGGADGLT |
| 827 | Vα without signal | VGLHLPTLSVQEGDNSIINCAYSNSASDYFIWYKQESGK |
| | peptide (SignalP) | GPQFIIDIRSNMDKRQGQRVTVLLNKTVKHLSLQIAATQ |
| | | PGDSAVYFCAENPGGGADGLTFGKGTHLIIQPNIQNPEP |
| | | AV |
| 828 | Vα only (without | MAGIRALFMYLWLQLDWVSRGESVGLHLPTLSVQEGDNS |
| | the Constant) | IINCAYSNSASDYFIWYKQESGKGPQFIIDIRSNMDKRQ |
| | | GQRVTVLLNKTVKHLSLQIAATQPGDSAVYFCAENPGGG |
| | | ADGLTFGKGTHLIIQPNIQNPEPAV |
| 829 | α chain with WT | MAGIRALFMYLWLQLDWVSRGESVGLHLPTLSVQEGDNS |
| | signal peptide | IINCAYSNSASDYFIWYKQESGKGPQFIIDIRSNMDKRQ |
| | and constant Cα | GQRVTVLLNKTVKHLSLQIAATQPGDSAVYFCAENPGGG |
| | | ADGLTFGKGTHLIIQPNIQNPEPAVNIQNPEPAVYQLKD |
| | | PRSQDSTLCLFTDFDSQINVPKTMESGTFITDKTVLDMK |
| | | AMDSKSNGAIAWSNQTSFTCQDIFKETNATYPSSDVPCD |
| | | ATLTEKSFETDMNLNFQNLLVIVLRILLLKVAGFNLLMT |
| | | LRLWSS |
| 830 | CDR1β | LGHDT |
| 831 | CDR2β | YNNKEL |
| 832 | CDR3β | ASSPGTGGYGYT |
| 833 | Vβ without signal | QTPKYLVTQMGNDKSIKCEQNLGHDTMYWYKQDSKKFLK |
| | peptide (SignalP) | IMFSYNNKELIINETVPNRFSPKSPDKAHLNLHINSLEL |
| | | GDSAVYFCASSPGTGGYGYTFGSGTRLTVVEDLRN |
| 834 | Vβ (without the Constant) | MGCRLLCCVVFCLLQAGPLDTAVSQTPKYLVTQMGNDKS |
| | | IKCEQNLGHDTMYWYKQDSKKFLKIMFSYNNKELIINET |
| | | VPNRFSPKSPDKAHLNLHINSLELGDSAVYFCASSPGTG |
| | | GYGYTFGSGTRLTVVEDLRN |
| 835 | β chain with WT | MGCRLLCCVVFCLLQAGPLDTAVSQTPKYLVTQMGNDKS |
| | signal peptide | IKCEQNLGHDTMYWYKQDSKKFLKIMFSYNNKELIINET |
| | and constant Cβ | VPNRFSPKSPDKAHLNLHINSLELGDSAVYFCASSPGTG |
| | | GYGYTFGSGTRLTVVEDLRNEDLRNVTPPKVSLFEPSKA |
| | | EIANKQKATLVCLARGFFPDHVELSWWVNGKEVHSGVST |
| | | DPQAYKESNYSYCLSSRLRVSATFWHNPRNHFRCQVQFH |
| | | GLSEEDKWPEGSPKPVTQNISAEAWGRADCGITSASYQQ |
| | | GVLSATILYEILLGKATLYAVLVSTLVVMAMVKRKNS |

In some embodiments, 5040-TCR45 interacts with and/or is specific for a peptide from gene LCK. In some embodiments, the peptide is from a neoantigen of LCK and has the amino acid change D326G (in which position 326 of the LCK protein is mutated from Asp to Gly). In some embodiments, 5040-TCR45 interacts with and/or is specific for the neoantigen in the context of HLA-B*44:03.

TABLE 51

| SEQ ID | | |
|---|---|---|
| NO. | Description | 5040-TCR47 |
| 836 | CDR1α | DSASNY |
| 837 | CDR2α | IRSNVGE |
| 838 | CDR3α | GGGGATNKLI |
| 839 | Vα without signal | ENVEQHPSTLSVQEGDSAVIKCTYSDSASNYFPWYKQEL |
| | peptide (SignalP) | GKRPQLIIDIRSNVGEKKDQRIAVTLNKTAKHFSLHITE |
| | | TQPEDSAVYFCGGGGATNKLIFGTGTLLAVQPNIQNPEP |
| | | AV |
| 840 | Vα only (without | MTSIRAVFIFLWLQLDLVNGENVEQHPSTLSVQEGDSAV |
| | the Constant) | IKCTYSDSASNYFPWYKQELGKRPQLIIDIRSNVGEKKD |
| | | QRIAVTLNKTAKHFSLHITETQPEDSAVYFCGGGGATNK |
| | | LIFGTGTLLAVQPNIQNPEPAV |
| 841 | α chain with WT | MTSIRAVFIFLWLQLDLVNGENVEQHPSTLSVQEGDSAV |
| | signal peptide | IKCTYSDSASNYFPWYKQELGKRPQLIIDIRSNVGEKKD |
| | and constant Cα | QRIAVTLNKTAKHFSLHITETQPEDSAVYFCGGGGATNK |
| | | LIFGTGTLLAVQPNIQNPEPAVNIQNPEPAVYQLKDPRS |
| | | QDSTLCLFTDFDSQINVPKTMESGTFITDKTVLDMKAMD |
| | | SKSNGAIAWSNQTSFTCQDIFKETNATYPSSDVPCDATL |
| | | TEKSFETDMNLNFQNLLVIVLRILLLKVAGFNLLMTLRL |
| | | WSS |
| 842 | CDR1β | SGHVS |
| 843 | CDR2β | FQNEAQ |
| 844 | CDR3β | ASNNEDGSYEQY |
| 845 | Vβ without signal | GVSQSPRYKVAKRGQDVALRCDPISGHVSLFWYQQALGQ |
| | peptide (SignalP) | GPEFLTYFQNEAQLDKSGLPSDRFFAERPEGSVSTLKIQ |
| | | RTQQEDSAVYLCASNNEDGSYEQYFGPGTRLTVTEDLRN |
| 846 | Vβ (without the Constant) | MGTRLLCWVVLGFLGTDHTGAGVSQSPRYKVAKRGQDVA |
| | | LRCDPISGHVSLFWYQQALGQGPEFLTYFQNEAQLDKSG |
| | | LPSDRFFAERPEGSVSTLKIQRTQQEDSAVYLCASNNED |
| | | GSYEQYFGPGTRLTVTEDLRN |
| 847 | β chain with WT | MGTRLLCWVVLGFLGTDHTGAGVSQSPRYKVAKRGQDVA |
| | signal peptide | LRCDPISGHVSLFWYQQALGQGPEFLTYFQNEAQLDKSG |
| | and constant Cβ | LPSDRFFAERPEGSVSTLKIQRTQQEDSAVYLCASNNED |
| | | GSYEQYFGPGTRLTVTEDLRNEDLRNVTPPKVSLFEPSK |
| | | AEIANKQKATLVCLARGFFPDHVELSWWVNGKEVHSGVS |
| | | TDPQAYKESNYSYCLSSRLRVSATFWHNPRNHFRCQVQF |
| | | HGLSEEDKWPEGSPKPVTQNISAEAWGRADCGITSASYQ |
| | | QGVLSATILYEILLGKATLYAVLVSTLVVMAMVKRKNS |

In some embodiments, 5040-TCR47 interacts with and/or is specific for a peptide from gene LCK. In some embodiments, the peptide is from a neoantigen of LCK and has the amino acid change D326G (in which position 326 of the LCK protein is mutated from Asp to Gly). In some embodiments, 5040-TCR47 interacts with and/or is specific for the neoantigen in the context of HLA-B*44:03.

TABLE 52

| SEQ ID | | |
|---|---|---|
| NO. | Description | 5040-TCR54 |
| 848 | CDR1α | TSINN |
| 849 | CDR2α | IRSNERE |
| 850 | CDR3α | ATGDDKII |
| 851 | Vα without signal | QQGEEDPQALSIQEGENATMNCSYKTSINNLQWYRQNSG |
| | peptide (SignalP) | RGLVHLILIRSNEREKHSGRLRVTLDTSKKSSSLLITAS |
| | | RAADTASYFCATGDDKIIFGKGTRLHILPNIQNPEPAV |
| 852 | Vα only (without | METLLGVSLVILWLQLARVNSQQGEEDPQALSIQEGENA |
| | the Constant) | TMNCSYKTSINNLQWYRQNSGRGLVHLILIRSNEREKHS |
| | | GRLRVTLDTSKKSSSLLITASRAADTASYFCATGDDKII |
| | | FGKGTRLHILPNIQNPEPAV |
| 853 | α chain with WT | METLLGVSLVILWLQLARVNSQQGEEDPQALSIQEGENA |
| | signal peptide | TMNCSYKTSINNLQWYRQNSGRGLVHLILIRSNEREKHS |
| | and constant Cα | GRLRVTLDTSKKSSSLLITASRAADTASYFCATGDDKII |
| | | FGKGTRLHILPNIQNPEPAVNIQNPEPAVYQLKDPRSQD |
| | | STLCLFTDFDSQINVPKTMESGTFITDKTVLDMKAMDSK |
| | | SNGAIAWSNQTSFTCQDIFKETNATYPSSDVPCDATLTE |
| | | KSFETDMNLNFQNLLVIVLRILLLKVAGFNLLMTLRLWS |
| | | S |
| 854 | CDR1β | LGHDT |
| 855 | CDR2β | YNNKEL |
| 856 | CDR3β | ASSQATGGEEAF |
| 857 | Vβ without signal | QTPKYLVTQMGNDKSIKCEQNLGHDTMYWYKQDSKKFLK |
| | peptide (SignalP) | IMFSYNNKELIINETVPNRFSPKSPDKAHLNLHINSLEL |
| | | GDSAVYFCASSQATGGEEAFFGQGTRLTVVEDLRN |
| 858 | Vβ (without the | MGCRLLCCVVFCLLQAGPLDTAVSQTPKYLVTQMGNDKS |
| | Constant) | IKCEQNLGHDTMYWYKQDSKKFLKIMFSYNNKELIINET |
| | | VPNRFSPKSPDKAHLNLHINSLELGDSAVYFCASSQATG |
| | | GEEAFFGQGTRLTVVEDLRN |
| 859 | β chain with WT | MGCRLLCCVVFCLLQAGPLDTAVSQTPKYLVTQMGNDKS |
| | signal peptide | IKCEQNLGHDTMYWYKQDSKKELKIMFSYNNKELIINET |
| | and constant Cβ | VPNRFSPKSPDKAHLNLHINSLELGDSAVYFCASSQATG |
| | | GEEAFFGQGTRLTVVEDLRNEDLRNVTPPKVSLFEPSKA |
| | | EIANKQKATLVCLARGFFPDHVELSWWVNGKEVHSGVST |
| | | DPQAYKESNYSYCLSSRLRVSATFWHNPRNHFRCQVQFH |
| | | GLSEEDKWPEGSPKPVTQNISAEAWGRADCGITSASYQQ |
| | | GVLSATILYEILLGKATLYAVLVSTLVVMAMVKRKNS |

In some embodiments, 5040-TCR54 interacts with and/or is specific for a peptide from gene LCK. In some embodiments, the peptide is from a neoantigen of LCK and has the amino acid change D326G (in which position 326 of the LCK protein is mutated from Asp to Gly). In some embodiments, 5040-TCR54 interacts with and/or is specific for the neoantigen in the context of HLA-B*44:03.

TABLE 53

| SEQ ID | | |
|---|---|---|
| NO. | Description | 5040-TCR106 |
| 860 | CDR1α | ATGYPS |
| 861 | CDR2α | ATKADDK |
| 862 | CDR3α | ALSDPFAQGGSEKLV |
| 863 | Vα without signal | DSVTQMEGPVTLSEEAFLTINCTYTATGYPSLFWYVQYP |
| | peptide (SignalP) | GEGLQLLLKATKADDKGSNKGFEATYRKETTSFHLEKGS |
| | | VQVSDSAVYFCALSDPFAQGGSEKLVFGKGTKLTVNPNI |
| | | QNPEPAV |
| 864 | Vα only (without the | MNYSPGLVSLILLLLGRTRGDSVTQMEGPVTLSEEAFLT |
| | Constant) | INCTYTATGYPSLFWYVQYPGEGLQLLLKATKADDKGSN |
| | | KGFEATYRKETTSFHLEKGSVQVSDSAVYFCALSDPFAQ |
| | | GGSEKLVFGKGTKLTVNPNIQNPEPAV |
| 865 | α chain with WT | MNYSPGLVSLILLLLGRTRGDSVTQMEGPVTLSEEAFLT |
| | signal peptide | INCTYTATGYPSLFWYVQYPGEGLQLLLKATKADDKGSN |
| | and constant Cα | KGFEATYRKETTSFHLEKGSVQVSDSAVYFCALSDPFAQ |
| | | GGSEKLVFGKGTKLTVNPNIQNPEPAVNIQNPEPAVYQL |
| | | KDPRSQDSTLCLFTDFDSQINVPKTMESGTFITDKTVLD |
| | | MKAMDSKSNGAIAWSNQTSFTCQDIFKETNATYPSSDVP |
| | | CDATLTEKSFETDMNLNFQNLLVIVLRILLLKVAGFNLL |
| | | MTLRLWSS |
| 866 | CDR1β | SGHAT |
| 867 | CDR2β | FQNNGV |
| 868 | CDR3β | ASSPRGGGNSPLH |
| 869 | Vβ without signal | GVAQSPRYKIIEKRQSVAFWCNPISGHATLYWYQQILGQ |
| | peptide (SignalP) | GPKLLIQFQNNGVVDDSQLPKDRFSAERLKGVDSTLKIQ |
| | | PAKLEDSAVYLCASSPRGGGNSPLHFGNGTRLTVTEDLR |
| | | N |
| 870 | Vβ (without the | MGTRLLCWAALCLLGAELTEAGVAQSPRYKIIEKRQSVA |
| | Constant) | FWCNPISGHATLYWYQQILGQGPKLLIQFQNNGVVDDSQ |
| | | LPKDRFSAERLKGVDSTLKIQPAKLEDSAVYLCASSPRG |
| | | GGNSPLHFGNGTRLTVTEDLRN |
| 871 | β chain with WT | MGTRLLCWAALCLLGAELTEAGVAQSPRYKIIEKRQSVA |
| | signal peptide | FWCNPISGHATLYWYQQILGQGPKLLIQFQNNGVVDDSQ |
| | and constant Cβ | LPKDRFSAERLKGVDSTLKIQPAKLEDSAVYLCASSPRG |
| | | GGNSPLHFGNGTRLTVTEDLRNEDLRNVTPPKVSLFEPS |
| | | KAEIANKQKATLVCLARGFFPDHVELSWWVNGKEVHSGV |
| | | STDPQAYKESNYSYCLSSRLRVSATFWHNPRNHFRCQVQ |
| | | FHGLSEEDKWPEGSPKPVTQNISAEAWGRADCGITSASY |
| | | QQGVLSATILYEILLGKATLYAVLVSTLVVMAMVKRKNS |

In some embodiments, 5040-TCR106 interacts with and/or is specific for a peptide from gene RCC1. In some embodiments, the peptide is from a neoantigen of RCC1 and has the amino acid change R430C (in which position 430 of the RCC1 protein is mutated from Arg to Cys). In some embodiments, 5040-TCR106 interacts with and/or is specific for 5 the neoantigen in the context of DPA1*01:03 and DPB1*02:01 or DPA1*02:01 and DPB1*02:01.

TABLE 54

| SEQ ID | | |
|---|---|---|
| NO. | Description | 5040-TCR128 |
| 872 | CDR1α | DRGSQS |
| 873 | CDR2α | IYSNGD |
| 874 | CDR3α | AVHMDSNYQLI |
| 875 | Vα without signal peptide | QQKEVEQNSGPLSVPEGAIASLNCTYSDRGSQSFFWYRQ |
| | (SignalP) | YSGKSPELIMFIYSNGDKEDGRFTAQLNKASQYVSLLIR |
| | | DSQPSDSATYLCAVHMDSNYQLIWGAGTKLIIKPNIQNP |
| | | EPAV |
| 876 | Vα only (without | MKSLRVLLVILWLQLSWVWSQQKEVEQNSGPLSVPEGAI |
| | the Constant) | ASLNCTYSDRGSQSFFWYRQYSGKSPELIMFIYSNGDKE |
| | | DGRFTAQLNKASQYVSLLIRDSQPSDSATYLCAVHMDSN |
| | | YQLIWGAGTKLIIKPNIQNPEPAV |
| 877 | α chain with WT | MKSLRVLLVILWLQLSWVWSQQKEVEQNSGPLSVPEGAI |
| | signal peptide | ASLNCTYSDRGSQSFFWYRQYSGKSPELIMFIYSNGDKE |
| | and constant Cα | DGRFTAQLNKASQYVSLLIRDSQPSDSATYLCAVHMDSN |
| | | YQLIWGAGTKLIIKPNIQNPEPAVNIQNPEPAVYQLKDP |
| | | RSQDSTLCLFTDFDSQINVPKTMESGTFITDKTVLDMKA |
| | | MDSKSNGAIAWSNQTSFTCQDIFKETNATYPSSDVPCDA |
| | | TLTEKSFETDMNLNFQNLLVIVLRILLLKVAGFNLLMTL |
| | | RLWSS |
| 878 | CDR1β | LNHDA |
| 879 | CDR2β | SQIVND |
| 880 | CDR3β | ASSIQGSNTEAF |
| 881 | Vβ without signal | GGITQSPKYLFRKEGQNVTLSCEQNLNHDAMYWYRQDPG |
| | peptide (SignalP) | QGLRLIYYSQIVNDFQKGDIAEGYSVSREKKESFPLTVT |
| | | SAQKNPTAFYLCASSIQGSNTEAFFGQGTRLTVVEDLRN |
| 882 | Vβ (without the Constant) | MSNQVLCCVVLCLLGANTVDGGITQSPKYLFRKEGQNVT |
| | | LSCEQNLNHDAMYWYRQDPGQGLRLIYYSQIVNDFQKGD |
| | | IAEGYSVSREKKESFPLTVTSAQKNPTAFYLCASSIQGS |
| | | NTEAFFGQGTRLTVVEDLRN |
| 883 | β chain with WT | MSNQVLCCVVLCLLGANTVDGGITQSPKYLFRKEGQNVT |
| | signal peptide | LSCEQNLNHDAMYWYRQDPGQGLRLIYYSQIVNDFQKGD |
| | and constant Cβ | IAEGYSVSREKKESFPLTVTSAQKNPTAFYLCASSIQGS |
| | | NTEAFFGQGTRLTVVEDLRNEDLRNVTPPKVSLFEPSKA |
| | | EIANKQKATLVCLARGFFPDHVELSWWVNGKEVHSGVST |
| | | DPQAYKESNYSYCLSSRLRVSATFWHNPRNHERCQVQFH |
| | | GLSEEDKWPEGSPKPVTQNISAEAWGRADCGITSASYQQ |
| | | GVLSATILYEILLGKATLYAVLVSTLVVMAMVKRKNS |

In some embodiments, 5040-TCR128 interacts with and/or is specific for a peptide from gene VARS. In some embodiments, the peptide is from a neoantigen of VARS and has the amino acid change R181C (in which position 181 of the VARS protein is mutated from Arg to Cys). In some embodiments, 5040-TCR128 interacts with and/or is specific for the neoantigen in the context of HLA-DRA and DRB1*04:01.

TABLE 55

| SEQ ID | | |
|---|---|---|
| NO. | Description | 5040-TCR39 |
| 884 | CDR1α | TSGFYG |
| 885 | CDR2α | NALDGL |
| 886 | CDR3α | AVGDSSYKLI |
| 887 | Vα without signal peptide | QSLEQPSEVTAVEGAIVQINCTYQTSGFYGLSWYQQHDG |
| | (SignalP) | GAPTFLSYNALDGLEETGRFSSFLSRSDSYGYLLLQELQ |
| | | MKDSASYFCAVGDSSYKLIFGSGTRLLVRPNIQNPEPAV |
| 888 | Vα only (without | MWGAFLLYVSMKMGGTAGQSLEQPSEVTAVEGAIVQINC |
| | the Constant) | TYQTSGFYGLSWYQQHDGGAPTFLSYNALDGLEETGRFS |
| | | SFLSRSDSYGYLLLQELQMKDSASYFCAVGDSSYKLIFG |
| | | SGTRLLVRPNIQNPEPAV |
| 889 | α chain with WT | MWGAFLLYVSMKMGGTAGQSLEQPSEVTAVEGAIVQINC |
| | signal peptide | TYQTSGFYGLSWYQQHDGGAPTFLSYNALDGLEETGRFS |
| | and constant Cα | SFLSRSDSYGYLLLQELQMKDSASYFCAVGDSSYKLIFG |
| | | SGTRLLVRPNIQNPEPAVNIQNPEPAVYQLKDPRSQDST |
| | | LCLFTDFDSQINVPKTMESGTFITDKTVLDMKAMDSKSN |
| | | GAIAWSNQTSFTCQDIFKETNATYPSSDVPCDATLTEKS |
| | | FETDMNLNFQNLLVIVLRILLLKVAGFNLLMTLRLWSS |
| 890 | CDR1β | MDHEN |
| 891 | CDR2β | SYDVKM |
| 892 | CDR3β | ASSFKGLEATDTQY |
| 893 | Vβ without signal peptide | SRYLVKRTGEKVFLECVQDMDHENMFWYRQDPGLGLRLI |
| | (SignalP) | YFSYDVKMKEKGDIPEGYSVSREKKERFSLILESASTNQ |
| | | TSMYLCASSFKGLEATDTQYFGPGTRLTVLEDLRN |
| 894 | Vβ (without the Constant) | MGIRLLCRVAFCFLAVGLVDVKVTQSSRYLVKRTGEKVF |
| | | LECVQDMDHENMFWYRQDPGLGLRLIYFSYDVKMKEKGD |
| | | IPEGYSVSREKKERFSLILESASTNQTSMYLCASSFKGL |
| | | EATDTQYFGPGTRLTVLEDLRN |
| 895 | β chain with WT | MGIRLLCRVAFCFLAVGLVDVKVTQSSRYLVKRTGEKVF |
| | signal peptide | LECVQDMDHENMFWYRQDPGLGLRLIYFSYDVKMKEKGD |
| | and constant Cβ | IPEGYSVSREKKERFSLILESASTNQTSMYLCASSFKGL |
| | | EATDTQYFGPGTRLTVLEDLRNEDLRNVTPPKVSLFEPS |
| | | KAELANKQKATLVCLARGFFPDHVELSWWVNGKEVHSGV |
| | | STDPQAYKESNYSYCLSSRLRVSATFWHNPRNHERCQVQ |
| | | FHGLSEEDKWPEGSPKPVTQNISAEAWGRADCGITSASY |
| | | QQGVLSATILYEILLGKATLYAVLVSTLVVMAMVKRKNS |

In some embodiments, 5040-TCR39 interacts with and/or is specific for a peptide from gene VARS. In some embodiments, the peptide is from a neoantigen of VARS and has the amino acid change R181C (in which position 181 of the VARS protein is mutated from Arg to Cys). In some embodiments, 5040-TCR39 interacts with and/or is specific for the 5 neoantigen in the context of HLA-DRA and DRB1*04:01.

TABLE 56

| SEQ ID | | |
|---|---|---|
| NO. | Description | 5040-TCR84 |
| 896 | CDR1α | TISGTDY |
| 897 | CDR2α | GLTSN |
| 898 | CDR3α | ILRDWSAGGTSYGKLT |
| 899 | Vα without signal | KTTQPNSMESNEEEPVHLPCNHSTISGTDYIHWYRQLPS |
| | peptide (SignalP) | QGPEYVIHGLTSNVNNRMASLAIAEDRKSSTLILHRATL |
| | | RDAAVYYCILRDWSAGGTSYGKLTFGQGTILTVHPNIQN |
| | | PEPAV |
| 900 | Vα only (without | MKLVTSITVLLSLGIMGDAKTTQPNSMESNEEEPVHLPC |
| | the Constant) | NHSTISGTDYIHWYRQLPSQGPEYVIHGLTSNVNNRMAS |
| | | LAIAEDRKSSTLILHRATLRDAAVYYCILRDWSAGGTSY |
| | | GKLTFGQGTILTVHPNIQNPEPAV |
| 901 | α chain with WT | MKLVTSITVLLSLGIMGDAKTTQPNSMESNEEEPVHLPC |
| | signal peptide | NHSTISGTDYIHWYRQLPSQGPEYVIHGLTSNVNNRMAS |
| | and constant Cα | LAIAEDRKSSTLILHRATLRDAAVYYCILRDWSAGGTSY |
| | | GKLTFGQGTILTVHPNIQNPEPAVNIQNPEPAVYQLKDP |
| | | RSQDSTLCLFTDFDSQINVPKTMESGTFITDKTVLDMKA |
| | | MDSKSNGAIAWSNQTSFTCQDIFKETNATYPSSDVPCDA |
| | | TLTEKSFETDMNLNFQNLLVIVLRILLLKVAGFNLLMTL |
| | | RLWSS |
| 902 | CDR1β | DFQATT |
| 903 | CDR2β | SNEGSKA |
| 904 | CDR3β | SADQGVTYGYT |
| 905 | Vβ without signal | AVVSQHPSRVICKSGTSVKIECRSLDFQATTMFWYRQFP |
| | peptide (SignalP) | KQSLMLMATSNEGSKATYEQGVEKDKFLINHASLTLSTL |
| | | TVTSAHPEDSSFYICSADQGVTYGYTFGSGTRLTVVEDL |
| | | RN |
| 906 | Vβ (without the | MLLLLLLLGPGSGLGAVVSQHPSRVICKSGTSVKIECRS |
| | Constant) | LDFQATTMFWYRQFPKQSLMLMATSNEGSKATYEQGVEK |
| | | DKFLINHASLTLSTLTVTSAHPEDSSFYICSADQGVTYG |
| | | YTFGSGTRLTVVEDLRN |
| 907 | β chain with WT | MLLLLLLLGPGSGLGAVVSQHPSRVICKSGTSVKIECRS |
| | signal peptide | LDFQATTMFWYRQFPKQSLMLMATSNEGSKATYEQGVEK |
| | and constant Cβ | DKFLINHASLTLSTLTVTSAHPEDSSFYICSADQGVTYG |
| | | YTFGSGTRLTVVEDLRNEDLRNVTPPKVSLFEPSKAEIA |
| | | NKQKATLVCLARGFFPDHVELSWWVNGKEVHSGVSTDPQ |
| | | AYKESNYSYCLSSRLRVSATFWHNPRNHFRCQVQFHGLS |
| | | EEDKWPEGSPKPVTQNISAEAWGRADCGITSASYQQGVL |
| | | SATILYEILLGKATLYAVLVSTLVVMAMVKRKNS |

In some embodiments, 5040-TCR84 interacts with and/or is specific for a peptide from gene VARS. In some embodiments, the peptide is from a neoantigen of VARS and has the amino acid change R181C (in which position 181 of the VARS protein is mutated from Arg to Cys). In some embodiments, 5040-TCR84 interacts with and/or is specific for the neoantigen in the context of HLA-DRA and DRB1*04:01.

TABLE 57

| SEQ ID | | |
|---|---|---|
| NO. | Description | 5040-TCR4 |
| 908 | CDR1α | SSVPPY |
| 909 | CDR2α | YTSAATLV |
| 910 | CDR3α | AVSRPTGTASKLT |
| 911 | Vα without | QSVTQLGSHVSVSEGALVLLRCNYSSSVPPYLFWYVQYP |
| | signal peptide | NQGLQLLLKYTSAATLVKGINGFEAEFKKSETSFHLTKP |
| | (SignalP) | SAHMSDAAEYFCAVSRPTGTASKLTFGTGTRLQVTLNIQ |
| | | NPEPAV |
| 912 | Vα only (without | MLLLLVPVLEVIFTLGGTRAQSVTQLGSHVSVSEGALVL |
| | Constant) | LRCNYSSSVPPYLFWYVQYPNQGLQLLLKYTSAATLVKG |
| | the | INGFEAEFKKSETSFHLTKPSAHMSDAAEYFCAVSRPTG |
| | | TASKLTFGTGTRLQVTLNIQNPEPAV |
| 913 | α chain with | MLLLLVPVLEVIFTLGGTRAQSVTQLGSHVSVSEGALVL |
| | WT signal | LRCNYSSSVPPYLFWYVQYPNQGLQLLLKYTSAATLVKG |
| | peptide and | INGFEAEFKKSETSFHLTKPSAHMSDAAEYFCAVSRPTG |
| | constant Cα | TASKLTFGTGTRLQVTLNIQNPEPAVNIQNPEPAVYQLK |
| | | DPRSQDSTLCLFTDFDSQINVPKTMESGTFITDKTVLDM |
| | | KAMDSKSNGAIAWSNQTSFTCQDIFKETNATYPSSDVPC |
| | | DATLTEKSFETDMNLNFQNLLVIVLRILLLKVAGFNLLM |
| | | TLRLWSS |
| 914 | CDR1β | DFQATT |
| 915 | CDR2β | SNEGSKA |
| 916 | CDR3β | SARAPSDSEAF |
| 917 | Vβ without | AVVSQHPSWVICKSGTSVKIECRSLDFQATTMFWYRQFP |
| | signal peptide | KQSLMLMATSNEGSKATYEQGVEKDKFLINHASLTLSTL |
| | (SignalP) | TVTSAHPEDSSFYICSARAPSDSEAFFGQGTRLTVVEDL |
| | | RN |
| 918 | Vβ (without the | MLLLLLLLGPGSGLGAVVSQHPSWVICKSGTSVKIECRS |
| | Constant) | LDFQATTMFWYRQFPKQSLMLMATSNEGSKATYEQGVEK |
| | | DKFLINHASLTLSTLTVTSAHPEDSSFYICSARAPSDSE |
| | | AFFGQGTRLTVVEDLRN |
| 919 | β chain with | MLLLLLLLGPGSGLGAVVSQHPSWVICKSGTSVKIECRS |
| | WT signal | LDFQATTMFWYRQFPKQSLMLMATSNEGSKATYEQGVEK |
| | peptide and | DKFLINHASLTLSTLTVTSAHPEDSSFYICSARAPSDSE |
| | constant Cβ | AFFGQGTRLTVVEDLRNEDLRNVTPPKVSLFEPSKAEIA |
| | | NKQKATLVCLARGFFPDHVELSWWVNGKEVHSGVSTDPQ |
| | | AYKESNYSYCLSSRLRVSATFWHNPRNHFRCQVQFHGLS |
| | | EEDKWPEGSPKPVTQNISAEAWGRADCGITSASYQQGVL |
| | | SATILYEILLGKATLYAVLVSTLVVMAMVKRKNS |

In some embodiments, 5040-TCR4 interacts with and/or is specific for a peptide from gene LCK. In some embodiments, the peptide is from a neoantigen of LCK and has the amino acid change D326G (in which position 326 of the LCK protein is mutated from Asp to Gly). In some embodiments, 5040-TCR4 interacts with and/or is specific for the neoantigen in the context of HLA-DRA and DRB1*04:01.

TABLE 58

| SEQ ID | | |
|---|---|---|
| NO. | Description | 8202-TCR17-1 |
| 920 | CDR1α | NTAFDY |
| 921 | CDR2α | IRPDVSE |
| 922 | CDR3α | AASMRFSNTGNQFY |
| 923 | Vα without signal | QQKEKSDQQQVKQSPQSLIVQKGGISIINCAYENTAFDY |
| | peptide (SignalP) | FPWYQQFPGKGPALLIAIRPDVSEKKEGRFTISFNKSAK |
| | | QFSLHIMDSQPGDSATYFCAASMRFSNTGNQFYFGTGTS |
| | | LTVIPNIQNPEPAV |
| 924 | Vα only (without | MDKILGASFLVLWLQLCWVSGQQKEKSDQQQVKQSPQSL |
| | Constant) | IVQKGGISIINCAYENTAFDYFPWYQQFPGKGPALLIAI |
| | the | RPDVSEKKEGRFTISFNKSAKQFSLHIMDSQPGDSATYF |
| | | CAASMRFSNTGNQFYFGTGTSLTVIPNIQNPEPAV |
| 925 | α chain with | MDKILGASFLVLWLQLCWVSGQQKEKSDQQQVKQSPQSL |
| | WT signal | IVQKGGISIINCAYENTAFDYFPWYQQFPGKGPALLIAI |
| | peptide and | RPDVSEKKEGRFTISFNKSAKQFSLHIMDSQPGDSATYF |
| | constant Cα | CAASMRFSNTGNQFYFGTGTSLTVIPNIQNPEPAVNIQN |
| | | PEPAVYQLKDPRSQDSTLCLFTDFDSQINVPKTMESGTF |
| | | ITDKTVLDMKAMDSKSNGAIAWSNQTSFTCQDIFKETNA |
| | | TYPSSDVPCDATLTEKSFETDMNLNFQNLLVIVLRILLL |
| | | KVAGFNLLMTLRLWSS |
| 926 | CDR1β | ENHRY |
| 927 | CDR2β | SYGVKD |
| 928 | CDR3β | AISEWGGNTIY |
| 929 | Vβ without signal | GITQSPRHKVTETGTPVTLRCHQTENHRYMYWYRQDPGH |
| | peptide (SignalP) | GLRLIHYSYGVKDTDKGEVSDGYSVSRSKTEDFLLTLES |
| | | ATSSQTSVYFCAISEWGGNTIYFGEGSWLTVVEDLRN |
| 930 | Vβ (without the | MGTRLFFYVALCLLWTGHMDAGITQSPRHKVTETGTPVT |
| | Constant) | LRCHQTENHRYMYWYRQDPGHGLRLIHYSYGVKDTDKGE |
| | | VSDGYSVSRSKTEDELLTLESATSSQTSVYFCAISEWGG |
| | | NTIYFGEGSWLTVVEDLRN |
| 931 | β chain with | MGTRLFFYVALCLLWTGHMDAGITQSPRHKVTETGTPVT |
| | WT signal | LRCHQTENHRYMYWYRQDPGHGLRLIHYSYGVKDTDKGE |
| | peptide and | VSDGYSVSRSKTEDFLLTLESATSSQTSVYFCAISEWGG |
| | constant Cβ | NTIYFGEGSWLTVVEDLRNEDLRNVTPPKVSLFEPSKAE |
| | | IANKQKATLVCLARGFFPDHVELSWWVNGKEVHSGVSTD |
| | | PQAYKESNYSYCLSSRLRVSATFWHNPRNHERCQVQFHG |
| | | LSEEDKWPEGSPKPVTQNISAEAWGRADCGITSASYQQG |
| | | VLSATILYEILLGKATLYAVLVSTLVVMAMVKRKNS |

In some embodiments, 8202-TCR17-1 interacts with and/or is specific for a peptide from gene ATP1A1. In some embodiments, the peptide is from a neoantigen of ATP1A1 and has the amino acid change A352T (in which position 352 of the ATP1A1 protein is mutated from Ala to Thr). In some embodiments, 8202-TCR17-1 interacts with and/or is specific for the neoantigen in the context of DPA1*01:03 and DPB1*10:01 or DPA1*02:01 and DPB1*10:01.

TABLE 59

| SEQ ID | | |
|---|---|---|
| NO. | Description | 8202-TCR9 |
| 932 | CDR1α | NTAFDY |
| 933 | CDR2α | IRPDVSE |
| 934 | CDR3α | AASYRGGNQFY |
| 935 | Vα without signal | QQKEKSDQQQVKQSPQSLIVQKGGIPIINCAYENTAFDY |
| | peptide(SignalP) | FPWYQQFPGKGPALLIAIRPDVSEKKEGRFTISFNKSAK |
| | | QFSLHIMDSQPGDSATYFCAASYRGGNQFYFGTGTSLTV |
| | | IPNIQNPEPAV |
| 936 | Vα only (without | MDKILGASFLVLWLQLCWVSGQQKEKSDQQQVKQSPQSL |
| | the Constant) | IVQKGGIPIINCAYENTAFDYFPWYQQFPGKGPALLIAI |
| | | RPDVSEKKEGRFTISFNKSAKQFSLHIMDSQPGDSATYF |
| | | CAASYRGGNQFYFGTGTSLTVIPNIQNPEPAV |
| 937 | α chain with | MDKILGASFLVLWLQLCWVSGQQKEKSDQQQVKQSPQSL |
| | WT signal | IVQKGGIPIINCAYENTAFDYFPWYQQFPGKGPALLIAI |
| | peptide and | RPDVSEKKEGRFTISFNKSAKQFSLHIMDSQPGDSATYF |
| | constant Cα | CAASYRGGNQFYFGTGTSLTVIPNIQNPEPAVNIQNPEP |
| | | AVYQLKDPRSQDSTLCLFTDFDSQINVPKTMESGTFITD |
| | | KTVLDMKAMDSKSNGAIAWSNQTSFTCQDIFKETNATYP |
| | | SSDVPCDATLTEKSFETDMNLNFQNLLVIVLRILLLKVA |
| | | GFNLLMTLRLWSS |
| 938 | CDR1β | DFQATT |
| 939 | CDR2β | SNEGSKA |
| 940 | CDR3β | SAFRTGDSEKLF |
| 941 | Vβ without | AVVSQHPSWVICKSGTSVKIECRSLDFQATTMFWYRQFP |
| | signal peptide | KQSLMLMATSNEGSKATYEQGVEKDKFLINHASLTLSTL |
| | (SignalP) | TVTSAHPEDSSFYICSAFRTGDSEKLFFGSGTQLSVLED |
| | | LRN |
| 942 | Vβ (without the | MLLLLLLLGPGSGLGAVVSQHPSWVICKSGTSVKIECRS |
| | Constant) | LDFQATTMFWYRQFPKQSLMLMATSNEGSKATYEQGVEK |
| | | DKFLINHASLTLSTLTVTSAHPEDSSFYICSAFRTGDSE |
| | | KLFFGSGTQLSVLEDLRN |
| 943 | Bβ chain with | MLLLLLLLGPGSGLGAVVSQHPSWVICKSGTSVKIECRS |
| | WT signal | LDFQATTMFWYRQFPKQSLMLMATSNEGSKATYEQGVEK |
| | peptide and | DKFLINHASLTLSTLTVTSAHPEDSSFYICSAFRTGDSE |
| | constant Cβ | KLFFGSGTQLSVLEDLRNEDLRNVTPPKVSLFEPSKAEI |
| | | ANKQKATLVCLARGFFPDHVELSWWVNGKEVHSGVSTDP |
| | | QAYKESNYSYCLSSRLRVSATFWHNPRNHFRCQVQFHGL |
| | | SEEDKWPEGSPKPVTQNISAEAWGRADCGITSASYQQGV |
| | | LSATILYEILLGKATLYAVLVSTLVVMAMVKRKNS |

In some embodiments, 8202-TCR9 interacts with and/or is specific for a peptide from gene ATP1A1. In some embodiments, the peptide is from a neoantigen of ATP1A1 and has the amino acid change A352T (in which position 352 of the ATP1A1 protein is mutated from Ala to Thr). In some embodiments, 8202-TCR9 interacts with and/or is specific for the neoantigen in the context of DPA1*01:03 and DPB1*10:01 or DPA1*02:01 and DPB1*10:01.

TABLE 60

| SEQ ID | | |
|---|---|---|
| NO. | Description | 5239-TCR45-2 |
| 944 | CDR1α | ATGYPS |
| 945 | CDR2α | ATKADDK |
| 946 | CDR3α | ALSETSGGGADGLT |
| 947 | Vα without signal | NSVTQMEGPVTLSEEAFLTINCTYTATGYPSLFWYVQYP |
| | peptide (SignalP) | GEGLQLLLKATKADDKGSNKGFEATYRKETTSFHLEKGS |
| | | VQVSDSAVYFCALSETSGGGADGLTFGKGTHLIIQPNIQ |
| | | NPEPAV |
| 948 | Vα only (without | MNYSPGLVSLILLLLGRTRGNSVTQMEGPVTLSEEAFLT |
| | the Constant) | INCTYTATGYPSLFWYVQYPGEGLQLLLKATKADDKGSN |
| | | KGFEATYRKETTSFHLEKGSVQVSDSAVYFCALSETSGG |
| | | GADGLTFGKGTHLIIQPNIQNPEPAV |
| 949 | α chain with | MNYSPGLVSLILLLLGRTRGNSVTQMEGPVTLSEEAFLT |
| | WT signal | INCTYTATGYPSLFWYVQYPGEGLQLLLKATKADDKGSN |
| | peptide and | KGFEATYRKETTSFHLEKGSVQVSDSAVYFCALSETSGG |
| | constant Cα | GADGLTFGKGTHLIIQPNIQNPEPAVNIQNPEPAVYQLK |
| | | DPRSQDSTLCLFTDFDSQINVPKTMESGTFITDKTVLDM |
| | | KAMDSKSNGAIAWSNQTSFTCQDIFKETNATYPSSDVPC |
| | | DATLTEKSFETDMNLNFQNLLVIVLRILLLKVAGFNLLM |
| | | TLRLWSS |
| 950 | CDR1β | DFQATT |
| 951 | CDR2β | SNEGSKA |
| 952 | CDR3β | SAVGGIYHNEQF |
| 953 | Vβ without signal | AVVSQHPSWVICKSGTSVKIECRSLDFQATTMFWYRQFP |
| | peptide (SignalP) | KQSLMLMATSNEGSKATYEQGVEKDKFLINHASLTLSTL |
| | | TVTSAHPEDSSFYICSAVGGIYHNEQFFGPGTRLTVLED |
| | | LRN |
| 954 | Vβ (without the | MLLLLLLLGPGSGLGAVVSQHPSWVICKSGTSVKIECRS |
| | Constant) | LDFQATTMFWYRQFPKQSLMLMATSNEGSKATYEQGVEK |
| | | DKFLINHASLTLSTLTVTSAHPEDSSFYICSAVGGIYHN |
| | | EQFFGPGTRLTVLEDLRN |
| 955 | β chain with | MLLLLLLLGPGSGLGAVVSQHPSWVICKSGTSVKIECRS |
| | WT signal | LDFQATTMFWYRQFPKQSLMLMATSNEGSKATYEQGVEK |
| | peptide and | DKFLINHASLTLSTLTVTSAHPEDSSFYICSAVGGIYHN |
| | constant Cβ | EQFFGPGTRLTVLEDLRNEDLRNVTPPKVSLFEPSKAEI |
| | | ANKQKATLVCLARGFFPDHVELSWWVNGKEVHSGVSTDP |
| | | QAYKESNYSYCLSSRLRVSATFWHNPRNHFRCQVQFHGL |
| | | SEEDKWPEGSPKPVTQNISAEAWGRADCGITSASYQQGV |
| | | LSATILYEILLGKATLYAVLVSTLVVMAMVKRKNS |

In some embodiments, 5239-TCR45-2 interacts with and/or is specific for a peptide from gene CRYBG3. In some embodiments, the peptide is from a neoantigen of CRYBG3 and has the amino acid change S316F (in which position 316 of the CRYBG3 protein is mutated from Ser to Phe). In some embodiments, 5239-TCR45-2 interacts with and/or is specific for the neoantigen in the context of DPA1*01:03 and DPB1*04:01 or DPA1*01:03 and DPB1*04:02.

TABLE 61

| SEQ ID | | |
|---|---|---|
| NO. | Description | 9976-TCR38-2 |
| 956 | CDR1α | SSVPPY |
| 957 | CDR2α | YTTGATLV |
| 958 | CDR3α | AVTEPGGYQKVT |
| 959 | Vα without signal | QSVTQLGSHVSVSEGALVLLRCNYSSSVPPYLFWYVQYP |
| | peptide (SignalP) | NQGLQLLLKYTTGATLVKGINGFEAEFKKSETSFHLTKP |
| | | SAHMSDAAEYFCAVTEPGGYQKVTFGTGTKLQVIPNIQN |
| | | PEPAV |
| 960 | Vα only (without | MLLLLVPVLEVIFTLGGTRAQSVTQLGSHVSVSEGALVL |
| | the Constant) | LRCNYSSSVPPYLFWYVQYPNQGLQLLLKYTTGATLVKG |
| | | INGFEAEFKKSETSFHLTKPSAHMSDAAEYFCAVTEPGG |
| | | YQKVTFGTGTKLQVIPNIQNPEPAV |
| 961 | α chain with | MLLLLVPVLEVIFTLGGTRAQSVTQLGSHVSVSEGALVL |
| | WT signal | LRCNYSSSVPPYLFWYVQYPNQGLQLLLKYTTGATLVKG |
| | peptide and | INGFEAEFKKSETSFHLTKPSAHMSDAAEYFCAVTEPGG |
| | constant Cα | YQKVTFGTGTKLQVIPNIQNPEPAVNIQNPEPAVYQLKD |
| | | PRSQDSTLCLFTDFDSQINVPKTMESGTFITDKTVLDMK |
| | | AMDSKSNGAIAWSNQTSFTCQDIFKETNATYPSSDVPCD |
| | | ATLTEKSFETDMNLNFQNLLVIVLRILLLKVAGFNLLMT |
| | | LRLWSS |
| 962 | CDR1β | DFQATT |
| 963 | CDR2β | SNEGSKA |
| 964 | CDR3β | SATGQHAGANVLT |
| 965 | Vβ without signal | AVVSQHPSRVICKSGTSVKIECRSLDFQATTMFWYRQFP |
| | peptide (SignalP) | KQSLMLMATSNEGSKATYEQGVEKDKFLINHASLTLSTL |
| | | TVTSAHPEDSSFYICSATGQHAGANVLTFGAGSRLTVLE |
| | | DLRN |
| 966 | Vβ (without the | MLLLLLLLGPGSGLGAVVSQHPSRVICKSGTSVKIECRS |
| | Constant) | LDFQATTMFWYRQFPKQSLMLMATSNEGSKATYEQGVEK |
| | | DKFLINHASLTLSTLTVTSAHPEDSSFYICSATGQHAGA |
| | | NVLTFGAGSRLTVLEDLRN |
| 967 | β chain with | MLLLLLLLGPGSGLGAVVSQHPSRVICKSGTSVKIECRS |
| | WT signal | LDFQATTMFWYRQFPKQSLMLMATSNEGSKATYEQGVEK |
| | peptide and | DKFLINHASLTLSTLTVTSAHPEDSSFYICSATGQHAGA |
| | constant Cβ | NVLTFGAGSRLTVLEDLRNEDLRNVTPPKVSLFEPSKAE |
| | | IANKQKATLVCLARGFFPDHVELSWWVNGKEVHSGVSTD |
| | | PQAYKESNYSYCLSSRLRVSATFWHNPRNHFRCQVQFHG |
| | | LSEEDKWPEGSPKPVTQNISAEAWGRADCGITSASYQQG |
| | | VLSATILYEILLGKATLYAVLVSTLVVMAMVKRKNS |

In some embodiments, 9976-TCR38-2 interacts with and/or is specific for a peptide from gene KRAS. In some embodiments, the peptide is from a neoantigen of KRAS and has the amino acid change G12V (in which position 12 of the KRAS protein is mutated from Gly to Val). In some embodiments, 9976-TCR38-2 interacts with and/or is specific for the neoantigen in the context of HLA-DRA and DRB1*07:01

TABLE 62

| SEQ ID | | |
|---|---|---|
| NO. | Description | 7014-TCR16 |
| 968 | CDR1α | SSVPPY |
| 969 | CDR2α | YTSAATLV |
| 970 | CDR3α | AVSERNNNARLM |
| 971 | Vα without signal | QSVTQLGSHVSVSEGALVLLRCNYSSSVPPYLFWYVQYP |
| | peptide (SignalP) | NQGLQLLLKYTSAATLVKGINGFEAEFKKSETSFHLTKP |
| | | SAHMSDAAEYFCAVSERNNNARLMFGDGTQLVVKPNIQN |
| | | PEPAV |
| 972 | Vα only (without | MLLLLVPVLEVIFTLGGTRAQSVTQLGSHVSVSEGALVL |
| | the Constant) | LRCNYSSSVPPYLFWYVQYPNQGLQLLLKYTSAATLVKG |
| | | INGFEAEFKKSETSFHLTKPSAHMSDAAEYFCAVSERNN |
| | | NARLMFGDGTQLVVKPNIQNPEPAV |
| 973 | α chain with | MLLLLVPVLEVIFTLGGTRAQSVTQLGSHVSVSEGALVL |
| | WT signal | LRCNYSSSVPPYLFWYVQYPNQGLQLLLKYTSAATLVKG |
| | peptide and | INGFEAEFKKSETSFHLTKPSAHMSDAAEYFCAVSERNN |
| | constant Cα | NARLMFGDGTQLVVKPNIQNPEPAVNIQNPEPAVYQLKD |
| | | PRSQDSTLCLFTDFDSQINVPKTMESGTFITDKTVLDMK |
| | | AMDSKSNGAIAWSNQTSFTCQDIFKETNATYPSSDVPCD |
| | | ATLTEKSFETDMNLNFQNLLVIVLRILLLKVAGFNLLMT |
| | | LRLWSS |
| 974 | CDR1β | SGHDT |
| 975 | CDR2β | YYEEEE |
| 976 | CDR3β | ASSHSGTYEQY |
| 977 | Vβ without signal | GVTQSPTHLIKTRGQQVTLRCSPKSGHDTVSWYQQALGQ |
| | peptide (SignalP) | GPQFIFQYYEEEERQRGNFPDRFSGHQFPNYSSELNVNA |
| | | LLLGDSALYLCASSHSGTYEQYFGPGTRLTVTEDLRN |
| 978 | Vβ (without the | MGPGLLCWALLCLLGAGLVDAGVTQSPTHLIKTRGQQVT |
| | Constant) | LRCSPKSGHDTVSWYQQALGQGPQFIFQYYEEEERQRGN |
| | | FPDRFSGHQFPNYSSELNVNALLLGDSALYLCASSHSGT |
| | | YEQYFGPGTRLTVTEDLRN |
| 979 | β chain with | MGPGLLCWALLCLLGAGLVDAGVTQSPTHLIKTRGQQVT |
| | WT signal | LRCSPKSGHDTVSWYQQALGQGPQFIFQYYEEEERQRGN |
| | peptide and | FPDRFSGHQFPNYSSELNVNALLLGDSALYLCASSHSGT |
| | constant Cβ | YEQYFGPGTRLTVTEDLRNEDLRNVTPPKVSLFEPSKAE |
| | | IANKQKATLVCLARGFFPDHVELSWWVNGKEVHSGVSTD |
| | | PQAYKESNYSYCLSSRLRVSATFWHNPRNHFRCQVQFHG |
| | | LSEEDKWPEGSPKPVTQNISAEAWGRADCGITSASYQQG |
| | | VLSATILYEILLGKATLYAVLVSTLVVMAMVKRKNS |

In some embodiments, 7014-TCR16 interacts with and/or is specific for a peptide from gene KRAS. In some embodiments, the peptide is from a neoantigen of KRAS and has the amino acid change G12V (in which position 12 of the KRAS protein is mutated from Gly to Val). In some embodiments, 7014-TCR16 interacts with and/or is specific for the neoantigen in the context of HLA-DRA and DRB1*07:01.

TABLE 63

| SEQ ID | | |
|---|---|---|
| NO. | Description | 7014-TCR51 |
| 980 | CDR1α | NSMFDY |
| 981 | CDR2α | ISSIKDK |
| 982 | CDR3α | AASVKTDKLI |
| 983 | Vα without signal | QQKNDDQQVKQNSPSLSVQEGRISILNCDYTNSMFDYFL |
| | peptide (SignalP) | WYKKYPAEGPTFLISISSIKDKNEDGRFTVFLNKSAKHL |
| | | SLHIVPSQPGDSAVYFCAASVKTDKLIFGTGTRLQVFPN |
| | | IQNPEPAV |
| 984 | Vα only (without | MAMLLGASVLILWLQPDWVNSQQKNDDQQVKQNSPSLSV |
| | the Constant) | QEGRISILNCDYTNSMEDYFLWYKKYPAEGPTFLISISS |
| | | IKDKNEDGRFTVFLNKSAKHLSLHIVPSQPGDSAVYFCA |
| | | ASVKTDKLIFGTGTRLQVFPNIQNPEPAV |
| 985 | α chain with | MAMLLGASVLILWLQPDWVNSQQKNDDQQVKQNSPSLSV |
| | WT signal | QEGRISILNCDYTNSMEDYFLWYKKYPAEGPTFLISISS |
| | peptide and | IKDKNEDGRFTVFLNKSAKHLSLHIVPSQPGDSAVYFCA |
| | constant Cα | ASVKTDKLIFGTGTRLQVFPNIQNPEPAVNIQNPEPAVY |
| | | QLKDPRSQDSTLCLFTDFDSQINVPKTMESGTFITDKTV |
| | | LDMKAMDSKSNGAIAWSNQTSFTCQDIFKETNATYPSSD |
| | | VPCDATLTEKSFETDMNLNFQNLLVIVLRILLLKVAGFN |
| | | LLMTLRLWSS |
| 986 | CDR1β | ENHRY |
| 987 | CDR2β | SYGVKD |
| 988 | CDR3β | AIREPLGLAKSSYNEQF |
| 989 | Vβ without signal | GITQSPRHKVTETGTPVTLRCHQTENHRYMYWYRQDPGH |
| | peptide (SignalP) | GLRLIHYSYGVKDTDKGEVSDGYSVSRSKTEDFLLTLES |
| | | ATSSQTSVYFCAIREPLGLAKSSYNEQFFGPGTRLTVLE |
| | | DLRN |
| 990 | Vβ (without the | MGTRLFFYVALCLLWTGHMDAGITQSPRHKVTETGTPVT |
| | Constant) | LRCHQTENHRYMYWYRQDPGHGLRLIHYSYGVKDTDKGE |
| | | VSDGYSVSRSKTEDFLLTLESATSSQTSVYFCAIREPLG |
| | | LAKSSYNEQFFGPGTRLTVLEDLRN |
| 991 | β chain with | MGTRLFFYVALCLLWTGHMDAGITQSPRHKVTETGTPVT |
| | WT signal | LRCHQTENHRYMYWYRQDPGHGLRLIHYSYGVKDTDKGE |
| | peptide and | VSDGYSVSRSKTEDELLTLESATSSQTSVYFCAIREPLG |
| | constant Cβ | LAKSSYNEQFFGPGTRLTVLEDLRNEDLRNVTPPKVSLF |
| | | EPSKAEIANKQKATLVCLARGFFPDHVELSWWVNGKEVH |
| | | SGVSTDPQAYKESNYSYCLSSRLRVSATFWHNPRNHFRC |
| | | QVQFHGLSEEDKWPEGSPKPVTQNISAEAWGRADCGITS |
| | | ASYQQGVLSATILYEILLGKATLYAVLVSTLVVMAMVKR |
| | | KNS |

In some embodiments, 7014-TCR51 interacts with and/or is specific for a peptide from gene KRAS. In some embodiments, the peptide is from a neoantigen of KRAS and has the amino acid change G12V (in which position 12 of the KRAS protein is mutated from Gly to Val). In some embodiments, 7014-TCR51 interacts with and/or is specific for the neoantigen in the context of HLA-DRA and DRB1*07:01.

TABLE 64

| SEQ ID | | |
|---|---|---|
| NO. | Description | 7014-TCR55 |
| 992 | CDR1α | SSVPPY |
| 993 | CDR2α | YTTGATLV |
| 994 | CDR3α | AVSGRNNNARLM |
| 995 | Vα without signal | QSVTQLGSHVSVSEGALVLLRCNYSSSVPPYLFWYVQYP |
| | peptide (SignalP) | NQGLQLLLKYTTGATLVKGINGFEAEFKKSETSFHLTKP |
| | | SAHMSDAAEYFCAVSGRNNNARLMFGDGTQLVVKPNIQN |
| | | PEPAV |
| 996 | Vα only (without | MLLLLVPVLEVIFTLGGTRAQSVTQLGSHVSVSEGALVL |
| | the Constant) | LRCNYSSSVPPYLFWYVQYPNQGLQLLLKYTTGATLVKG |
| | | INGFEAEFKKSETSFHLTKPSAHMSDAAEYFCAVSGRNN |
| | | NARLMFGDGTQLVVKPNIQNPEPAV |
| 997 | α chain with | MLLLLVPVLEVIFTLGGTRAQSVTQLGSHVSVSEGALVL |
| | WT signal | LRCNYSSSVPPYLFWYVQYPNQGLQLLLKYTTGATLVKG |
| | peptide and | INGFEAEFKKSETSFHLTKPSAHMSDAAEYFCAVSGRNN |
| | constant Cα | NARLMFGDGTQLVVKPNIQNPEPAVNIQNPEPAVYQLKD |
| | | PRSQDSTLCLFTDFDSQINVPKTMESGTFITDKTVLDMK |
| | | AMDSKSNGAIAWSNQTSFTCQDIFKETNATYPSSDVPCD |
| | | ATLTEKSFETDMNLNFQNLLVIVLRILLLKVAGFNLLMT |
| | | LRLWSS |
| 998 | CDR1β | SGHDT |
| 999 | CDR2β | YYEEEE |
| 1000 | CDR3β | ASRRQGSYEQY |
| 1001 | Vβ without signal | GVTQSPTHLIKTRGQQVTLRCSPKSGHDTVSWYQQALGQ |
| | peptide (SignalP) | GPQFIFQYYEEEERQRGNFPDRFSGHQFPNYSSELNVNA |
| | | LLLGDSALYLCASRRQGSYEQYFGPGTRLTVTEDLRN |
| 1002 | Vβ (without the | MGPGLLCWALLCLLGAGLVDAGVTQSPTHLIKTRGQQVT |
| | Constant) | LRCSPKSGHDTVSWYQQALGQGPQFIFQYYEEEERQRGN |
| | | FPDRFSGHQFPNYSSELNVNALLLGDSALYLCASRRQGS |
| | | YEQYFGPGTRLTVTEDLRN |
| 1003 | β chain with | MGPGLLCWALLCLLGAGLVDAGVTQSPTHLIKTRGQQVT |
| | WT signal | LRCSPKSGHDTVSWYQQALGQGPQFIFQYYEEEERQRGN |
| | peptide and | FPDRFSGHQFPNYSSELNVNALLLGDSALYLCASRRQGS |
| | constant Cβ | YEQYFGPGTRLTVTEDLRNEDLRNVTPPKVSLFEPSKAE |
| | | IANKQKATLVCLARGFFPDHVELSWWVNGKEVHSGVSTD |
| | | PQAYKESNYSYCLSSRLRVSATFWHNPRNHFRCQVQFHG |
| | | LSEEDKWPEGSPKPVTQNISAEAWGRADCGITSASYQQG |
| | | VLSATILYEILLGKATLYAVLVSTLVVMAMVKRKNS |

In some embodiments, 7014-TCR55 interacts with and/or is specific for a peptide from gene KRAS. In some embodiments, the peptide is from a neoantigen of KRAS and has the amino acid change G12V (in which position 12 of the KRAS protein is mutated from Gly to Val). In some embodiments, 7014-TCR55 interacts with and/or is specific for the neoantigen in the context of HLA-DRA and DRB1*07:01.

TABLE 65

| SEQ ID NO. | Description | CLL000160-TCR70 |
|---|---|---|
| 1025 | CDR1α | NSMFDY |
| 1026 | CDR2α | ISSIKDK |
| 1027 | CDR3a | AARKLQGGKLI |
| 1028 | Vα without signal | QQKNDDQQVKQNSPSLSVQEGRISILNCDYTNSMFDYF |
| | peptide (SignalP) | LWYKKYPAEGPTFLISISSIKDKNEDGRFTVFLNKSAK |
| | | HLSLHIVPSQPGDSAVYFCAARKLQGGKLIFGQGTELS |
| | | VKPNIQNPEPAV |
| 1029 | Vα only (without | MAMLLGASVLILWLQPDWVNSQQKNDDQQVKQNSPSLS |
| | the Constant) | VQEGRISILNCDYTNSMEDYFLWYKKYPAEGPTFLISI |
| | | SSIKDKNEDGRFTVFLNKSAKHLSLHIVPSQPGDSAVY |
| | | FCAARKLQGGKLIFGQGTELSVKPNIQNPEPAV |
| 1030 | α chain with WT | MAMLLGASVLILWLQPDWVNSQQKNDDQQVKQNSPSLS |
| | signal peptide and | VQEGRISILNCDYTNSMEDYFLWYKKYPAEGPTFLISI |
| | constant Cα | SSIKDKNEDGRFTVFLNKSAKHLSLHIVPSQPGDSAVY |
| | | FCAARKLQGGKLIFGQGTELSVKPNIQNPEPAVNIQNP |
| | | EPAVYQLKDPRSQDSTLCLFTDFDSQINVPKTMESGTF |
| | | ITDKTVLDMKAMDSKSNGAIAWSNQTSFTCQDIFKETN |
| | | ATYPSSDVPCDATLTEKSFETDMNLNFQNLLVIVLRIL |
| | | LLKVAGFNLLMTLRLWSS |
| 1031 | CDR1β | SGHVS |
| 1032 | CDR2β | FNYEAQ |
| 1033 | CDR3β | ASSLWLGALSSGANVLT |
| 1034 | Vβ without signal | GVSQSPRYKVTKRGQDVALRCDPISGHVSLYWYRQALG |
| | peptide (SignalP) | QGPEFLTYFNYEAQQDKSGLPNDRESAERPEGSISTLT |
| | | IQRTEQRDSAMYRCASSLWLGALSSGANVLTFGAGSRL |
| | | TVLEDLRN |
| 1035 | Vβ (without the | MGTSLLCWVVLGFLGTDHTGAGVSQSPRYKVTKRGQDV |
| | Constant) | ALRCDPISGHVSLYWYRQALGQGPEFLTYFNYEAQQDK |
| | | SGLPNDRFSAERPEGSISTLTIQRTEQRDSAMYRCASS |
| | | LWLGALSSGANVLTFGAGSRLTVLEDLRN |
| 1036 | β chain with WT | MGTSLLCWVVLGFLGTDHTGAGVSQSPRYKVTKRGQDV |
| | signal peptide and | ALRCDPISGHVSLYWYRQALGQGPEFLTYFNYEAQQDK |
| | constant Cβ | SGLPNDRFSAERPEGSISTLTIQRTEQRDSAMYRCASS |
| | | LWLGALSSGANVLTFGAGSRLTVLEDLRNEDLRNVTPP |
| | | KVSLFEPSKAEIANKQKATLVCLARGFFPDHVELSWWV |
| | | NGKEVHSGVSTDPQAYKESNYSYCLSSRLRVSATFWHN |
| | | PRNHFRCQVQFHGLSEEDKWPEGSPKPVTQNISAEAWG |
| | | RADCGITSASYQQGVLSATILYEILLGKATLYAVLVST |
| | | LVVMAMVKRKNS |

In some embodiments, CLL000160-TCR70 interacts with and/or is specific for a peptide from gene KRAS. In some embodiments, the peptide is from a neoantigen of KRAS and has the amino acid change G12V (in which position 12 of the KRAS protein is mutated from Gly to Val). In some embodiments, CLL000160-TCR70 interacts with and/or is specific for the neoantigen in the context of HLA-DRA and DRB1*07:01.

TABLE 66

| SEQ ID NO. | Description | 8202-TCR8 |
|---|---|---|
| 1037 | CDR1α | NSAFQY |
| 1038 | CDR2α | TYSSGN |
| 1039 | CDR3α | AMSYNTNAGKST |
| 1040 | Vα without signal | QQKEVEQDPGPLSVPEGAIVSLNCTYSNSAFQYEMWYRQ |
| | peptide (SignalP) | YSRKGPELLMYTYSSGNKEDGRFTAQVDKSSKYISLFIR |
| | | DSQPSDSATYLCAMSYNTNAGKSTFGDGTTLTVKPNIQN |
| | | PEPAV |
| 1041 | Vα only (without | MMKSLRVLLVILWLQLSWVWSQQKEVEQDPGPLSVPEGA |
| | the Constant) | IVSLNCTYSNSAFQYFMWYRQYSRKGPELLMYTYSSGNK |
| | | EDGRFTAQVDKSSKYISLFIRDSQPSDSATYLCAMSYNT |
| | | NAGKSTFGDGTTLTVKPNIQNPEPAV |
| 1042 | α chain with WT | MMKSLRVLLVILWLQLSWVWSQQKEVEQDPGPLSVPEGA |
| | signal peptide and | IVSLNCTYSNSAFQYFMWYRQYSRKGPELLMYTYSSGNK |
| | constant Cα | EDGRFTAQVDKSSKYISLFIRDSQPSDSATYLCAMSYNT |
| | | NAGKSTFGDGTTLTVKPNIQNPEPAVNIQNPEPAVYQLK |
| | | DPRSQDSTLCLFTDFDSQINVPKTMESGTFITDKTVLDM |
| | | KAMDSKSNGAIAWSNQTSFTCQDIFKETNATYPSSDVPC |
| | | DATLTEKSFETDMNLNFQNLLVIVLRILLLKVAGFNLLM |
| | | TLRLWSS |
| 1043 | CDR1β | SGHVS |
| 1044 | CDR2β | FQNEAQ |
| 1045 | CDR3β | ASSVRDRANEQF |
| 1046 | Vβ without signal | GVSQSPRYKVAKRGQDVALRCDPISGHVSLFWYQQALGQ |
| | peptide (SignalP) | GPEFLTYFQNEAQLDKSGLPSDRFFAERPEGSVSTLKIQ |
| | | RTQQEDSAVYLCASSVRDRANEQFFGPGTRLTVLEDLRN |
| 1047 | Vβ (without the | MGTRLLCWVVLGFLGTDHTGAGVSQSPRYKVAKRGQDVA |
| | Constant) | LRCDPISGHVSLFWYQQALGQGPEFLTYFQNEAQLDKSG |
| | | LPSDRFFAERPEGSVSTLKIQRTQQEDSAVYLCASSVRD |
| | | RANEQFFGPGTRLTVLEDLRN |
| 1048 | β chain with WT | MGTRLLCWVVLGFLGTDHTGAGVSQSPRYKVAKRGQDVA |
| | signal peptide and | LRCDPISGHVSLFWYQQALGQGPEFLTYFQNEAQLDKSG |
| | constant Cβ | LPSDRFFAERPEGSVSTLKIQRTQQEDSAVYLCASSVRD |
| | | RANEQFFGPGTRLTVLEDLRNEDLRNVTPPKVSLFEPSK |
| | | AEIANKQKATLVCLARGFFPDHVELSWWVNGKEVHSGVS |
| | | TDPQAYKESNYSYCLSSRLRVSATFWHNPRNHFRCQVQF |
| | | HGLSEEDKWPEGSPKPVTQNISAEAWGRADCGITSASYQ |
| | | QGVLSATILYEILLGKATLYAVLVSTLVVMAMVKRKNS |

In some embodiments, 8202-TCR8 interacts with and/or is specific for a peptide from gene ABCC3. In some embodiments, the peptide is from a neoantigen of ABCC3 and has the amino acid change A86V (in which position 86 of the ABCC3 protein is mutated from Ala to Val). In some embodiments, 8202-TCR8 interacts with and/or is specific for the neoantigen in the context of HLA-A*03:01 and A*11:01.

TABLE 67

| SEQ ID NO. | Description | 8202-TCR11 |
|---|---|---|
| 1049 | CDR1α | TSGFYG |
| 1050 | CDR2α | NALDGL |
| 1051 | CDR3α | AVPSSNTGKLI |
| 1052 | Vα without signal | QSLEQPSEVTAVEGAIVQINCTYQTSGFYGLSWYQQHDG |
| | peptide (SignalP) | GAPTFLSYNALDGLEETGRFSSFLSRSDSYGYLLLQELQ |
| | | MKDSASYFCAVPSSNTGKLIFGQGTTLQVKPNIQNPEPA |
| | | V |
| 1053 | Vα only (without | MWGAFLLYVSMKMGGTAGQSLEQPSEVTAVEGAIVQINC |
| | the Constant) | TYQTSGFYGLSWYQQHDGGAPTFLSYNALDGLEETGRFS |
| | | SFLSRSDSYGYLLLQELQMKDSASYFCAVPSSNIGKLIF |
| | | GQGTTLQVKPNIQNPEPAV |
| 1054 | α chain with WT | MWGAFLLYVSMKMGGTAGQSLEQPSEVTAVEGAIVQINC |
| | signal peptide and | TYQTSGFYGLSWYQQHDGGAPTFLSYNALDGLEETGRFS |
| | constant Cα | SFLSRSDSYGYLLLQELQMKDSASYFCAVPSSNTGKLIF |
| | | GQGTTLQVKPNIQNPEPAVNIQNPEPAVYQLKDPRSQDS |
| | | TLCLFTDFDSQINVPKTMESGTFITDKTVLDMKAMDSKS |
| | | NGAIAWSNFTSFTCQDIFKETNATYPSSDVPCDATLTEK |
| | | SFETDMNLNFQNLLVIVLRILLLKVAGFNLLMTLRLWSS |
| 1055 | CDR1β | SGHRS |
| 1056 | CDR2β | YFSETQ |
| 1057 | CDR3β | ASSLRTGEKLF |
| 1058 | Vβ without signal | GVTQTPRYLIKTRGQQVTLSCSPISGHRSVSWYQQTPGQ |
| | peptide (SignalP) | GLQFLFEYFSETQRNKGNFPGRFSGRQFSNSRSEMNVST |
| | | LELGDSALYLCASSLRTGEKLFFGSGTQLSVLEDLRN |
| 1059 | Vβ (without the | MGSRLLCWVLLCLLGAGPVKAGVTQTPRYLIKTRGQQVT |
| | Constant) | LSCSPISGHRSVSWYQQTPGQGLQFLFEYFSETQRNKGN |
| | | FPGRFSGRQFSNSRSEMNVSTLELGDSALYLCASSLRTG |
| | | EKLFFGSGTQLSVLEDLRN |
| 1060 | β chain with WT | MGSRLLCWVLLCLLGAGPVKAGVTQTPRYLIKTRGQQVT |
| | signal peptide and | LSCSPISGHRSVSWYQQTPGQGLQFLFEYFSETQRNKGN |
| | constant Cβ | FPGRFSGRQFSNSRSEMNVSTLELGDSALYLCASSLRTG |
| | | EKLFFGSGTQLSVLEDLRNEDLRNVTPPKVSLFEPSKAE |
| | | IANKQKATLVCLARGFFPDHVELSWWVNGKEVHSGVSTD |
| | | PQAYKESNYSYCLSSRLRVSATFWHNPRNHFRCQVQFHG |
| | | LSEEDKWPEGSPKPVTQNISAEAWGRADCGITSASYQQG |
| | | VLSATILYEILLGKATLYAVLVSTLVVMAMVKRKNS |

In some embodiments, 8202-TCR11 interacts with and/or is specific for a peptide from gene ABCC3. In some embodiments, the peptide is from a neoantigen of ABCC3 and has the amino acid change A86V (in which position 86 of the ABCC3 protein is mutated from Ala to Val). In some embodiments, 8202-TCR11 interacts with and/or is specific for the neoantigen in the context of HLA-A*03:01.

TABLE 68

| SEQ ID NO. | Description | 0359-TCR1 |
|---|---|---|
| 1061 | CDR1α | NSMFDY |
| 1062 | CDR2α | ISSIKDK |
| 1063 | CDR3α | AASALTGNQFY |
| 1064 | Vα without signal | QQKNDDQQVKQNSPSLSVQEGRISILNCDYTNSMEDYF |
| | peptide (SignalP) | LWYKKYPAEGPTFLISISSIKDKNEDGRFTVELNKSAK |
| | | HLSLHIVPSQPGDSAVYFCAASALTGNQFYFGTGTSLT |
| | | VIPNIQNPEPAV |
| 1065 | Vα only (without | MAMLLGASVLILWLQPDWVNSQQKNDDQQVKQNSPSLS |
| | the Constant) | VQEGRISILNCDYTNSMEDYFLWYKKYPAEGPTFLISI |
| | | SSIKDKNEDGRFTVFLNKSAKHLSLHIVPSQPGDSAVY |
| | | FCAASALTGNQFYFGTGTSLTVIPNIQNPEPAV |
| 1066 | α chain with WT | MAMLLGASVLILWLQPDWVNSQQKNDDQQVKQNSPSLS |
| | signal peptide and | VQEGRISILNCDYTNSMEDYFLWYKKYPAEGPTFLISI |
| | constant Cα | SSIKDKNEDGRFTVFLNKSAKHLSLHIVPSQPGDSAVY |
| | | FCAASALTGNQFYFGTGTSLTVIPNIQNPEPAVNIQNP |
| | | EPAVYQLKDPRSQDSTLCLFTDFDSQINVPKTMESGTF |
| | | ITDKTVLDMKAMDSKSNGAIAWSNQTSFTCQDIFKETN |
| | | ATYPSSDVPCDATLTEKSFETDMNLNFQNLLVIVLRIL |
| | | LLKVAGFNLLMTLRLWSS |
| 1067 | CDR1β | SGHAT |
| 1068 | CDR2β | FQNNGV |
| 1069 | CDR3β | ASSTGTGPRPQH |
| 1070 | Vβ without signal | GVAQSPRYKIIEKRQSVAFWCNPISGHATLYWYQQILG |
| | peptide (SignalP) | QGPKLLIQFQNNGVVDDSQLPKDRFSAERLKGVDSTLK |
| | | IQPAKLEDSAVYLCASSTGTGPRPQHFGDGIRLSILED |
| | | LRN |
| 1071 | Vβ (without the | MGTRLLCWAALCLLGAELTEAGVAQSPRYKIIEKRQSV |
| | Constant) | AFWCNPISGHATLYWYQQILGQGPKLLIQFQNNGVVDD |
| | | SQLPKDRFSAERLKGVDSTLKIQPAKLEDSAVYLCASS |
| | | TGTGPRPQHFGDGTRLSILEDLRN |
| 1072 | β chain with WT | MGTRLLCWAALCLLGAELTEAGVAQSPRYKIIEKRQSV |
| | signal peptide and | AFWCNPISGHATLYWYQQILGQGPKLLIQFQNNGVVDD |
| | constant Cβ | SQLPKDRFSAERLKGVDSTLKIQPAKLEDSAVYLCASS |
| | | TGTGPRPQHFGDGTRLSILEDLRNEDLRNVTPPKVSLF |
| | | EPSKAEIANKQKATLVCLARGFFPDHVELSWWVNGKEV |
| | | HSGVSTDPQAYKESNYSYCLSSRLRVSATFWHNPRNHF |
| | | RCQVQFHGLSEEDKWPEGSPKPVTQNISAEAWGRADCG |
| | | ITSASYQQGVLSATILYEILLGKATLYAVLVSTLVVMA |
| | | MVKRKNS |

In some embodiments, 0359-TCR1 interacts with and/or is specific for a neoantigen in the context of HLA-A*30:01.

TABLE 69

| SEQ ID NO. | Description | 0359-TCR15 |
|---|---|---|
| 1073 | CDR1α | TSESDYY |
| 1074 | CDR2α | QEAYKQQN |
| 1075 | CDR3α | AFSGNTPLV |
| 1076 | Vα without signal | QTVTQSQPEMSVQEAETVTLSCTYDTSESDYYLFWYKQ |
| | peptide (SignalP) | PPSRQMILVIRQEAYKQQNATENRFSVNFQKAAKSFSL |
| | | KISDSQLGDAAMYFCAFSGNTPLVFGKGTRLSVIANIQ |
| | | NPEPAV |
| 1077 | Vα only (without | MACPGFLWALVISTCLEFSMAQTVTQSQPEMSVQEAET |
| | the Constant) | VTLSCTYDTSESDYYLFWYKQPPSRQMILVIRQEAYKQ |
| | | QNATENRFSVNFQKAAKSFSLKISDSQLGDAAMYFCAF |
| | | SGNTPLVFGKGTRLSVIANIQNPEPAV |
| 1078 | α chain with WT | MACPGFLWALVISTCLEFSMAQTVTQSQPEMSVQEAET |
| | signal peptide and | VTLSCTYDTSESDYYLFWYKQPPSRQMILVIRQEAYKQ |
| | constant Cα | QNATENRFSVNFQKAAKSFSLKISDSQLGDAAMYFCAF |
| | | SGNTPLVFGKGTRLSVIANIQNPEPAVNIQNPEPAVYQ |
| | | LKDPRSQDSTLCLFTDFDSQINVPKTMESGTFITDKTV |
| | | LDMKAMDSKSNGAIAWSNQTSFTCQDIFKETNATYPSS |
| | | DVPCDATLTEKSFETDMNLNFQNLLVIVLRILLLKVAG |
| | | FNLLMTLRLWSS |
| 1079 | CDR1β | SEHNR |
| 1080 | CDR2β | FQNEAQ |
| 1081 | CDR3β | ASSSLRTSGTYNEQF |
| 1082 | Vβ without signal | DTGVSQDPRHKITKRGQNVTFRCDPISEHNRLYWYRQT |
| | peptide (SignalP) | LGQGPEFLTYFQNEAQLEKSRLLSDRFSAERPKGSEST |
| | | LEIQRTEQGDSAMYLCASSSLRTSGTYNEQFFGPGTRL |
| | | TVLEDLRN |
| 1083 | Vβ (without the | MGTSLLCWMALCLLGADHADTGVSQDPRHKITKRGQNV |
| | Constant) | TFRCDPISEHNRLYWYRQTLGQGPEFLTYFQNEAQLEK |
| | | SRLLSDRFSAERPKGSESTLEIQRTEQGDSAMYLCASS |
| | | SLRTSGTYNEQFFGPGTRLTVLEDLRN |
| 1084 | β chain with WT | MGTSLLCWMALCLLGADHADTGVSQDPRHKITKRGQNV |
| | signal peptide and | TFRCDPISEHNRLYWYRQTLGQGPEFLTYFQNEAQLEK |
| | constant Cβ | SRLLSDRFSAERPKGSESTLEIQRTEQGDSAMYLCASS |
| | | SLRTSGTYNEQFFGPGTRLTVLEDLRNEDLRNVTPPKV |
| | | SLEEPSKAEIANKQKATLVCLARGFFPDHVELSWWVNG |
| | | KEVHSGVSTDPQAYKESNYSYCLSSRLRVSATFWHNPR |
| | | NHFRCQVQFHGLSEEDKWPEGSPKPVTQNISAEAWGRA |
| | | DCGITSASYQQGVLSATILYEILLGKATLYAVLVSTLV |
| | | VMAMVKRKNS |

In some embodiments, 0359-TCR15 interacts with and/or is specific for a neoantigen in the context of HLA-A*30:01.

TABLE 70

| SEQ ID NO. | Description | 0359-TCR43 |
|---|---|---|
| 1085 | CDR1α | DSSSTY |
| 1086 | CDR2α | IFSNMDM |
| 1087 | CDR3α | AESMGSDSWGKFQ |
| 1088 | Vα without signal | EDVEQSLFLSVREGDSSVINCTYTDSSSTYLYWYKQEP |
| | peptide (SignalP) | GAGLQLLTYIFSNMDMKQDQRLTVLLNKKDKHLSLRIA |
| | | DTQTGDSAIYFCAESMGSDSWGKFQFGAGTQVVVTPNI |
| | | QNPEPAV |
| 1089 | Vα only (without | MKTFAGFSFLFLWLQLDCMSRGEDVEQSLFLSVREGDS |
| | the Constant) | SVINCTYTDSSSTYLYWYKQEPGAGLQLLTYIFSNMDM |
| | | KQDQRLTVLLNKKDKHLSLRIADTQTGDSAIYFCAESM |
| | | GSDSWGKFQFGAGTQVVVTPNIQNPEPAV |
| 1090 | α chain with WT | MKTFAGFSFLFLWLQLDCMSRGEDVEQSLFLSVREGDS |
| | signal peptide and | SVINCTYTDSSSTYLYWYKQEPGAGLQLLTYIFSNMDM |
| | constant Cα | KQDQRLTVLLNKKDKHLSLRIADTQTGDSAIYFCAESM |
| | | GSDSWGKFQFGAGTQVVVTPNIQNPEPAVNIQNPEPAV |
| | | YQLKDPRSQDSTLCLFTDFDSQINVPKTMESGTFITDK |
| | | TVLDMKAMDSKSNGAIAWSNQTSFTCQDIFKETNATYP |
| | | SSDVPCDATLTEKSFETDMNLNFQNLLVIVLRILLLKV |
| | | AGFNLLMTLRLWSS |
| 1091 | CDR1β | MNHNS |
| 1092 | CDR2β | SASEGT |
| 1093 | CDR3β | ASTPGQFSYNEQF |
| 1094 | Vβ without signal | GVTQTPKFQVLKTGQSMTLQCAQDMNHNSMYWYRQDPG |
| | peptide (SignalP) | MGLRLIYYSASEGTTDKGEVPNGYNVSRLNKREFSLRL |
| | | ESAAPSQTSVYFCASTPGQFSYNEQFFGPGTRLTVLED |
| | | LRN |
| 1095 | Vβ (without the | MSIGLLCCVAFSLLWASPVNAGVTQTPKFQVLKTGQSM |
| | Constant) | TLQCAQDMNHNSMYWYRQDPGMGLRLIYYSASEGTTDK |
| | | GEVPNGYNVSRLNKREFSLRLESAAPSQTSVYFCASTP |
| | | GQFSYNEQFFGPGTRLTVLEDLRN |
| 1096 | β chain with WT | MSIGLLCCVAFSLLWASPVNAGVTQTPKFQVLKTGQSM |
| | signal peptide and | TLQCAQDMNHNSMYWYRQDPGMGLRLIYYSASEGTTDK |
| | constant Cβ | GEVPNGYNVSRLNKREFSLRLESAAPSQTSVYFCASTP |
| | | GQFSYNEQFFGPGTRLTVLEDLRNEDLRNVTPPKVSLF |
| | | EPSKAEIANKQKATLVCLARGFFPDHVELSWWVNGKEV |
| | | HSGVSTDPQAYKESNYSYCLSSRLRVSATFWHNPRNHF |
| | | RCQVQFHGLSEEDKWPEGSPKPVTQNISAEAWGRADCG |
| | | ITSASYQQGVLSATILYEILLGKATLYAVLVSTLVVMA |
| | | MVKRKNS |

In some embodiments, 0359-TCR43 interacts with and/or is specific for a neoantigen in the context of HLA-C*12:03.

TABLE 71

| SEQ ID NO. | Description | 0359-TCR45 |
|---|---|---|
| 1097 | CDR1α | NSAFQY |
| 1098 | CDR2α | TYSSGN |
| 1099 | CDR3α | AMRGDSSYKLI |
| 1100 | Vα without signal | QQKEVEQDPGPLSVPEGAIVSLNCTYSNSAFQYFMWYR |
| | peptide (SignalP) | QYSRKGPELLMYTYSSGNKEDGRETAQVDKSSKYISLF |
| | | IRDSQPSDSATYLCAMRGDSSYKLIFGSGTRLLVRPNI |
| | | QNPEPAV |
| 1101 | Vα only (without | MMKSLRVLLVILWLQLSWVWSQQKEVEQDPGPLSVPEG |
| | the Constant) | AIVSLNCTYSNSAFQYFMWYRQYSRKGPELLMYTYSSG |
| | | NKEDGRFTAQVDKSSKYISLFIRDSQPSDSATYLCAMR |
| | | GDSSYKLIFGSGTRLLVRPNIQNPEPAV |
| 1102 | α chain with WT | MMKSLRVLLVILWLQLSWVWSQQKEVEQDPGPLSVPEG |
| | signal peptide and | AIVSLNCTYSNSAFQYFMWYRQYSRKGPELLMYTYSSG |
| | constant Cα | NKEDGRFTAQVDKSSKYISLFIRDSQPSDSATYLCAMR |
| | | GDSSYKLIFGSGTRLLVRPNIQNPEPAVNIQNPEPAVY |
| | | QLKDPRSQDSTLCLFTDFDSQINVPKTMESGTFITDKT |
| | | VLDMKAMDSKSNGAIAWSNQTSFTCQDIFKETNATYPS |
| | | SDVPCDATLTEKSFETDMNLNFQNLLVIVLRILLLKVA |
| | | GENLLMTLRLWSS |
| 1103 | CDR1β | LNHDA |
| 1104 | CDR2β | SQIVND |
| 1105 | CDR3β | ASTQFQGKGNQPQH |
| 1106 | Vβ without signal | GITQSPKYLFRKEGQNVTLSCEQNLNHDAMYWYRQDPG |
| | peptide (SignalP) | QGLRLIYYSQIVNDFQKGDIAEGYSVSREKKESFPLTV |
| | | TSAQKNPTAFYLCASTQFQGKGNQPQHFGDGIRLSILE |
| | | DLRN |
| 1107 | Vβ (without the | MDTRLLCCAVICLLGADTVDGGITQSPKYLFRKEGQNV |
| | Constant) | TLSCEQNLNHDAMYWYRQDPGQGLRLIYYSQIVNDFQK |
| | | GDIAEGYSVSREKKESFPLTVTSAQKNPTAFYLCASTQ |
| | | FQGKGNQPQHFGDGIRLSILEDLRN |
| 1108 | β chain with WT | MDTRLLCCAVICLLGADTVDGGITQSPKYLFRKEGQNV |
| | signal peptide and | TLSCEQNLNHDAMYWYRQDPGQGLRLIYYSQIVNDFQK |
| | constant Cβ | GDIAEGYSVSREKKESFPLTVTSAQKNPTAFYLCASTQ |
| | | FQGKGNQPQHFGDGIRLSILEDLRNEDLRNVTPPKVSL |
| | | FEPSKAEIANKQKATLVCLARGFFPDHVELSWWVNGKE |
| | | VHSGVSTDPQAYKESNYSYCLSSRLRVSATFWHNPRNH |
| | | FRCQVQFHGLSEEDKWPEGSPKPVTQNISAEAWGRADC |
| | | GITSASYQQGVLSATILYEILLGKATLYAVLVSTLVVM |
| | | AMVKRKNS |

In some embodiments, 0359-TCR45 interacts with and/or is specific for a neoantigen in the context of HLA-A*30:01.

TABLE 72

| SEQ ID NO. | Description | 3489-TCR16 |
|---|---|---|
| 1109 | CDR1α | SIFNT |
| 1110 | CDR2α | LYKAGEL |
| 1111 | CDR3α | AGRHGSSNTGKLI |
| 1112 | Vα without signal | QQLNQSPQSMFIQEGEDVSMNCTSSSIFNTWLWYKQDP |
| | peptide (SignalP) | GEGPVLLIALYKAGELTSNGRLTAQFGITRKDSFLNIS |
| | | ASIPSDVGIYFCAGRHGSSNIGKLIFGQGTTLQVKPNI |
| | | QNPEPAV |
| 1113 | Vα only (without | MLLEHLLIILWMQLTWVSGQQLNQSPQSMFIQEGEDVS |
| | the Constant) | MNCTSSSIFNTWLWYKQDPGEGPVLLIALYKAGELTSN |
| | | GRLTAQFGITRKDSFLNISASIPSDVGIYFCAGRHGSS |
| | | NTGKLIFGQGTTLQVKPNIQNPEPAV |
| 1114 | α chain with WT | MLLEHLLIILWMQLTWVSGQQLNQSPQSMFIQEGEDVS |
| | signal peptide and | MNCTSSSIFNTWLWYKQDPGEGPVLLIALYKAGELTSN |
| | constant Cα | GRLTAQFGITRKDSFLNISASIPSDVGIYFCAGRHGSS |
| | | NTGKLIFGQGTTLQVKPNIQNPEPAVNIQNPEPAVYQL |
| | | KDPRSQDSTLCLFTDFDSQINVPKTMESGTFITDKTVL |
| | | DMKAMDSKSNGAIAWSNQTSFTCQDIFKETNATYPSSD |
| | | VPCDATLTEKSFETDMNLNFQNLLVIVLRILLLKVAGF |
| | | NLLMTLRLWSS |
| 1115 | CDR1β | LNHDA |
| 1116 | CDR2β | SQIVND |
| 1117 | CDR3β | ASSITGGYEQY |
| 1118 | Vβ without signal | GITQSPKYLFRKEGQNVTLSCEQNLNHDAMYWYRQDPG |
| | peptide (SignalP) | QGLRLIYYSQIVNDFQKGDIAEGYSVSREKKESFPLTV |
| | | TSAQKNPTAFYLCASSITGGYEQYFGPGTRLTVTEDLR |
| | | N |
| 1119 | Vβ (without the | MSNQVLCCVVLCFLGANTVDGGITQSPKYLFRKEGQNV |
| | Constant) | TLSCEQNLNHDAMYWYRQDPGQGLRLIYYSQIVNDFQK |
| | | GDIAEGYSVSREKKESFPLTVTSAQKNPTAFYLCASSI |
| | | TGGYEQYFGPGTRLTVTEDLRN |
| 1120 | β chain with WT | MSNQVLCCVVLCFLGANTVDGGITQSPKYLFRKEGQNV |
| | signal peptide and | TLSCEQNLNHDAMYWYRQDPGQGLRLIYYSQIVNDFQK |
| | constant Cβ | GDIAEGYSVSREKKESFPLTVTSAQKNPTAFYLCASSI |
| | | TGGYEQYFGPGTRLTVTEDLRNEDLRNVTPPKVSLFEP |
| | | SKAEIANKQKATLVCLARGFFPDHVELSWWVNGKEVHS |
| | | GVSTDPQAYKESNYSYCLSSRLRVSATFWHNPRNHFRC |
| | | QVQFHGLSEEDKWPEGSPKPVTQNISAEAWGRADCGIT |
| | | SASYQQGVLSATILYEILLGKATLYAVLVSTLVVMAMV |
| | | KRKNS |

In some embodiments, 3489-TCR16 interacts with and/or is specific for a neoantigen in the context of HLA-B*51:01.

TABLE 73

| SEQ ID NO. | Description | 7014-TCR44 |
|---|---|---|
| 1121 | CDR1α | NSMFDY |
| 1122 | CDR2α | ISSIKDK |
| 1123 | CDR3α | AALSSGSARQLT |
| 1124 | Vα without signal | QQKNDDQQVKQNSPSLSVQEGRISILNCDYTNSMEDYF |
| | peptide (SignalP) | LWYKKYPAEGPTFLISISSIKDKNEDGRFTVFLNKSAK |
| | | HLSLHIVPSQPGDSAVYFCAALSSGSARQLTFGSGTQL |
| | | TVLPNIQNPEPAV |
| 1125 | Vα only (without | MAMLLGASVLILWLQPDWVNSQQKNDDQQVKQNSPSLS |
| | the Constant) | VQEGRISILNCDYTNSMFDYFLWYKKYPAEGPTFLISI |
| | | SSIKDKNEDGRFTVELNKSAKHLSLHIVPSQPGDSAVY |
| | | FCAALSSGSARQLTFGSGTQLTVLPNIQNPEPAV |
| 1126 | α chain with WT | MAMLLGASVLILWLQPDWVNSQQKNDDQQVKQNSPSLS |
| | signal peptide and | VQEGRISILNCDYTNSMFDYFLWYKKYPAEGPTFLISI |
| | constant Cα | SSIKDKNEDGRFTVFLNKSAKHLSLHIVPSQPGDSAVY |
| | | FCAALSSGSARQLTFGSGTQLTVLPNIQNPEPAVNIQN |
| | | PEPAVYQLKDPRSQDSTLCLFTDFDSQINVPKTMESGT |
| | | FITDKTVLDMKAMDSKSNGAIAWSNQTSFTCQDIFKET |
| | | NATYPSSDVPCDATLTEKSFETDMNLNFQNLLVIVLRI |
| | | LLLKVAGFNLLMTLRLWSS |
| 1127 | CDR1β | SGHRS |
| 1128 | CDR2β | YFSETQ |
| 1129 | CDR3β | ASSVSLGDTGELF |
| 1130 | Vβ without signal | GVTQTPRYLIKTRGQQVTLSCSPISGHRSVSWYQQTPG |
| | peptide (SignalP) | QGLQFLFEYFSETQRNKGNFPGRESGRQFSNSRSEMNV |
| | | STLELGDSALYLCASSVSLGDTGELFFGEGSRLTVLED |
| | | LRN |
| 1131 | Vβ (without the | MGSRLLCWVLLCLLGAGPVKAGVTQTPRYLIKTRGQQV |
| | Constant) | TLSCSPISGHRSVSWYQQTPGQGLQFLFEYFSETQRNK |
| | | GNFPGRESGRQFSNSRSEMNVSTLELGDSALYLCASSV |
| | | SLGDTGELFFGEGSRLTVLEDLRN |
| 1132 | β chain with WT | MGSRLLCWVLLCLLGAGPVKAGVTQTPRYLIKTRGQQV |
| | signal peptide and | TLSCSPISGHRSVSWYQQTPGQGLQFLFEYFSETQRNK |
| | constant Cβ | GNFPGRESGRQFSNSRSEMNVSTLELGDSALYLCASSV |
| | | SLGDTGELFFGEGSRLTVLEDLRNEDLRNVTPPKVSLF |
| | | EPSKAEIANKQKATLVCLARGFFPDHVELSWWVNGKEV |
| | | HSGVSTDPQAYKESNYSYCLSSRLRVSATFWHNPRNHF |
| | | RCQVQFHGLSEEDKWPEGSPKPVTQNISAEAWGRADCG |
| | | ITSASYQQGVLSATILYEILLGKATLYAVLVSTLVVMA |
| | | MVKRKNS |

In some embodiments, 7014-TCR44 interacts with and/or is specific for a neoantigen in the context of DRB3*02:02.

TABLE 74

| SEQ ID NO. | Description | 3080-TCR14 |
|---|---|---|
| 1133 | CDR1α | KALYS |
| 1134 | CDR2α | LLKGGEQ |
| 1135 | CDR3α | GTNGVGGADGLT |
| 1136 | Vα without signal | QQPVQSPQAVILREGEDAVINCSSSKALYSVHWYRQKH |
| | peptide (SignalP) | GEAPVFLMILLKGGEQKGHEKISASFNEKKQQSSLYLT |
| | | ASQLSYSGTYFCGTNGVGGADGLTFGKGTHLIIQPNIQ |
| | | NPEPAV |
| 1137 | Vα only (without | METLLKVLSGTLLWQLTWVRSQQPVQSPQAVILREGED |
| | the Constant) | AVINCSSSKALYSVHWYRQKHGEAPVELMILLKGGEQK |
| | | GHEKISASFNEKKQQSSLYLTASQLSYSGTYFCGTNGV |
| | | GGADGLTFGKGTHLIIQPNIQNPEPAV |
| 1138 | α chain with WT | METLLKVLSGTLLWQLTWVRSQQPVQSPQAVILREGED |
| | signal peptide and | AVINCSSSKALYSVHWYRQKHGEAPVELMILLKGGEQK |
| | constant Cα | GHEKISASFNEKKQQSSLYLTASQLSYSGTYFCGINGV |
| | | GGADGLTFGKGTHLIIQPNIQNPEPAVNIQNPEPAVYQ |
| | | LKDPRSQDSTLCLFTDFDSQINVPKTMESGTFITDKTV |
| | | LDMKAMDSKSNGAIAWSNQTSFTCQDIFKETNATYPSS |
| | | DVPCDATLTEKSFETDMNLNFQNLLVIVLRILLLKVAG |
| | | FNLLMTLRLWSS |
| 1139 | CDR1β | WNHNN |
| 1140 | CDR2β | SYGVHD |
| 1141 | CDR3β | ASSESSNWGMNTEAF |
| 1142 | Vβ without signal | EITQSPRHKITETGRQVTLACHQTWNHNNMFWYRQDLG |
| | peptide (SignalP) | HGLRLIHYSYGVHDTNKGEVSDGYSVSRSNTEDLPLTL |
| | | ESAASSQTSVYFCASSESSNWGMNTEAFFGQGTRLTVV |
| | | EDLRN |
| 1143 | Vβ (without the | MGTRLFFYVALCLLWAGHRDAEITQSPRHKITETGRQV |
| | Constant) | TLACHQTWNHNNMFWYRQDLGHGLRLIHYSYGVHDTNK |
| | | GEVSDGYSVSRSNTEDLPLTLESAASSQTSVYFCASSE |
| | | SSNWGMNTEAFFGQGTRLTVVEDLRN |
| 1144 | β chain with WT | MGTRLFFYVALCLLWAGHRDAEITQSPRHKITETGRQV |
| | signal peptide and | TLACHQTWNHNNMFWYRQDLGHGLRLIHYSYGVHDTNK |
| | constant Cβ | GEVSDGYSVSRSNTEDLPLTLESAASSQTSVYFCASSE |
| | | SSNWGMNTEAFFGQGTRLTVVEDLRNEDLRNVTPPKVS |
| | | LFEPSKAEIANKQKATLVCLARGFFPDHVELSWWVNGK |
| | | EVHSGVSTDPQAYKESNYSYCLSSRLRVSATFWHNPRN |
| | | HFRCQVQFHGLSEEDKWPEGSPKPVTQNISAEAWGRAD |
| | | CGITSASYQQGVLSATILYEILLGKATLYAVLVSTLVV |
| | | MAMVKRKNS |

In some embodiments, 3080-TCR14 interacts with and/or is specific for a neoantigen in the context of HLA-A*02:01, A*30:01, B*13:02 or C*06:02.

TABLE 75

| SEQ ID NO. | Description | 3080-TCR39 |
|---|---|---|
| 1145 | CDR1α | NSASDY |
| 1146 | CDR2α | IRSNMDK |
| 1147 | CDR3α | AEISGTYKYI |
| 1148 | Vα without signal | ENVGLHLPTLSVQEGDNSIINCAYSNSASDYFIWYKQE |
| | peptide (SignalP) | SGKGPQFIIDIRSNMDKRQGQRVTVLLNKTVKHLSLQI |
| | | AATQPGDSAVYFCAEISGTYKYIFGTGTRLKVLANIQN |
| | | PEPAV |
| 1149 | Vα only (without | MAGIRALFMYLWLQLDWVSRGENVGLHLPTLSVQEGDN |
| | the Constant) | SIINCAYSNSASDYFIWYKQESGKGPQFIIDIRSNMDK |
| | | RQGQRVTVLLNKTVKHLSLQIAATQPGDSAVYFCAEIS |
| | | GTYKYIFGTGTRLKVLANIQNPEPAV |
| 1150 | α chain with WT | MAGIRALFMYLWLQLDWVSRGENVGLHLPTLSVQEGDN |
| | signal peptide and | SIINCAYSNSASDYFIWYKQESGKGPQFIIDIRSNMDK |
| | constant Cα | RQGQRVTVLLNKTVKHLSLQIAATQPGDSAVYFCAEIS |
| | | GTYKYIFGTGTRLKVLANIQNPEPAVNIQNPEPAVYQL |
| | | KDPRSQDSTLCLFTDFDSQINVPKTMESGTFITDKTVL |
| | | DMKAMDSKSNGAIAWSNQTSFTCQDIFKETNATYPSSD |
| | | VPCDATLTEKSFETDMNLNFQNLLVIVLRILLLKVAGF |
| | | NLLMTLRLWSS |
| 1151 | CDR1β | SQVTM |
| 1152 | CDR2β | ANQGSEA |
| 1153 | CDR3β | SVAAGTNYGYT |
| 1154 | Vβ without signal | AVISQKPSRDICQRGTSLTIQCQVDSQVTMMFWYRQQP |
| | peptide (SignalP) | GQSLTLIATANQGSEATYESGFVIDKFPISRPNLTFST |
| | | LTVSNMSPEDSSIYLCSVAAGTNYGYTFGSGTRLTVVE |
| | | DLRN |
| 1155 | Vβ (without the | MLSLLLLLLGLGSVFSAVISQKPSRDICQRGTSLTIQC |
| | Constant) | QVDSQVTMMFWYRQQPGQSLTLIATANQGSEATYESGF |
| | | VIDKFPISRPNLTFSTLTVSNMSPEDSSIYLCSVAAGT |
| | | NYGYTFGSGTRLTVVEDLRN |
| 1156 | β chain with WT | MLSLLLLLLGLGSVFSAVISQKPSRDICQRGTSLTIQC |
| | signal peptide and | QVDSQVTMMFWYRQQPGQSLTLIATANQGSEATYESGF |
| | constant Cβ | VIDKFPISRPNLTFSTLTVSNMSPEDSSIYLCSVAAGT |
| | | NYGYTFGSGTRLTVVEDLRNEDLRNVTPPKVSLFEPSK |
| | | AEIANKQKATLVCLARGFFPDHVELSWWVNGKEVHSGV |
| | | STDPQAYKESNYSYCLSSRLRVSATFWHNPRNHFRCQV |
| | | QFHGLSEEDKWPEGSPKPVTQNISAEAWGRADCGITSA |
| | | SYQQGVLSATILYEILLGKATLYAVLVSTLVVMAMVKR |
| | | KNS |

In some embodiments, 3080-TCR39 interacts with and/or is specific for a neoantigen in the context of DPA1*01:03, DPA1*02:01, DPB1*04:02; DQA1*01:02, DQB1*06:03; DRB3*02:02, DRB1*15:01, DRB5*01:01.

TABLE 76

| SEQ ID NO. | Description | CLL000032-TCR2 |
|---|---|---|
| 1157 | CDR1α | TSENNYY |
| 1158 | CDR2α | QEAYKQQN |
| 1159 | CDR3α | AFNSFSGAGSYQLT |
| 1160 | Va without signal | QTVTQSQPEMSVQEAETVTLSCTYDTSENNYYLFWYKQ |
| | peptide (SignalP) | PPSRQMILVIRQEAYKQQNATENRFSVNFQKAAKSFSL |
| | | KISDSQLGDTAMYFCAFNSFSGAGSYQLTFGKGTKLSV |
| | | IPNIQNPEPAV |
| 1161 | Vα only (without | MTRVSLLWAVVVSTCLESGMAQTVTQSQPEMSVQEAET |
| | the Constant) | VTLSCTYDTSENNYYLFWYKQPPSRQMILVIRQEAYKQ |
| | | QNATENRFSVNFQKAAKSFSLKISDSQLGDTAMYFCAF |
| | | NSFSGAGSYQLTFGKGTKLSVIPNIQNPEPAV |
| 1162 | α chain with WT | MTRVSLLWAVVVSTCLESGMAQTVTQSQPEMSVQEAET |
| | signal peptide and | VTLSCTYDTSENNYYLFWYKQPPSRQMILVIRQEAYKQ |
| | constant Cα | QNATENRFSVNFQKAAKSFSLKISDSQLGDTAMYFCAF |
| | | NSFSGAGSYQLTFGKGTKLSVIPNIQNPEPAVNIQNPE |
| | | PAVYQLKDPRSQDSTLCLFTDFDSQINVPKTMESGTFI |
| | | TDKTVLDMKAMDSKSNGAIAWSNQTSFTCQDIFKETNA |
| | | TYPSSDVPCDATLTEKSFETDMNLNFQNLLVIVLRILL |
| | | LKVAGFNLLMTLRLWSS |
| 1163 | CDR1β | SGHNT |
| 1164 | CDR2β | YYREEE |
| 1165 | CDR3β | ASSSLQETQY |
| 1166 | Vβ without signal | GVTQSPTHLIKTRGQQVTLRCSSQSGHNTVSWYQQALG |
| | peptide (SignalP) | QGPQFIFQYYREEENGRGNFPPRFSGLQFPNYSSFLNV |
| | | NALELDDSALYLCASSSLQETQYFGPGTRLLVLEDLRN |
| 1167 | Vβ (without the | MGPGLLCWALLCLLGAGSVETGVTQSPTHLIKTRGQQV |
| | Constant) | TLRCSSQSGHNTVSWYQQALGQGPQFIFQYYREEENGR |
| | | GNFPPRFSGLQFPNYSSFLNVNALELDDSALYLCASSS |
| | | LQETQYFGPGTRLLVLEDLRN |
| 1168 | β chain with WT | MGPGLLCWALLCLLGAGSVETGVTQSPTHLIKTRGQQV |
| | signal peptide and | TLRCSSQSGHNTVSWYQQALGQGPQFIFQYYREEENGR |
| | constant Cβ | GNFPPRFSGLQFPNYSSFLNVNALELDDSALYLCASSS |
| | | LQETQYFGPGTRLLVLEDLRNEDLRNVTPPKVSLFEPS |
| | | KAEIANKQKATLVCLARGFFPDHVELSWWVNGKEVHSG |
| | | VSTDPQAYKESNYSYCLSSRLRVSATFWHNPRNHFRCQ |
| | | VQFHGLSEEDKWPEGSPKPVTQNISAEAWGRADCGITS |
| | | ASYQQGVLSATILYEILLGKATLYAVLVSTLVVMAMVK |
| | | RKNS |

In some embodiments, CLL000032-TCR2 interacts with and/or is specific for a peptide from gene TP53. In some embodiments, the peptide is from a neoantigen of TP53 and has the amino acid change R175H (in which position 175 of the TP53 protein is mutated from Arg to His). In some embodiments, CLL000032-TCR2 interacts with and/or is specific for the neoantigen in the context of HLA-A*02:01.

TABLE 77

| SEQ ID NO. | Description | CLL000032-TCR37 |
|---|---|---|
| 1169 | CDR1α | TSENNYY |
| 1170 | CDR2α | QEAYKQQN |
| 1171 | CDR3α | AFMKYTGGGNKLT |
| 1172 | Vα without signal | QTVTQSQPEMSVQEAETVTLSCTYDTSENNYYLFWYKQ |
| | peptide (SignalP) | PPSRQMILVIRQEAYKQQNATENRFSVNFQKAAKSFSL |
| | | KISDSQLGDTAMYFCAFMKYTGGGNKLTFGTGTQLKVE |
| | | LNIQNPEPAV |
| 1173 | Vα only (without | MTRVSLLWAVVVSTCLESGMAQTVTQSQPEMSVQEAET |
| | the Constant) | VTLSCTYDTSENNYYLFWYKQPPSRQMILVIRQEAYKQ |
| | | QNATENRFSVNFQKAAKSFSLKISDSQLGDTAMYFCAF |
| | | MKYTGGGNKLTFGTGTQLKVELNIQNPEPAV |
| 1174 | α chain with WT | MTRVSLLWAVVVSTCLESGMAQTVTQSQPEMSVQEAET |
| | signal peptide and | VTLSCTYDTSENNYYLFWYKQPPSRQMILVIRQEAYKQ |
| | constant Cα | QNATENRFSVNFQKAAKSFSLKISDSQLGDTAMYFCAF |
| | | MKYTGGGNKLTFGTGTQLKVELNIQNPEPAVNIQNPEP |
| | | AVYQLKDPRSQDSTLCLFTDFDSQINVPKTMESGTFIT |
| | | DKTVLDMKAMDSKSNGAIAWSNQTSFTCQDIFKETNAT |
| | | YPSSDVPCDATLTEKSFETDMNLNFQNLLVIVLRILLL |
| | | KVAGENLLMTLRLWSS |
| 1175 | CDR1β | SNHLY |
| 1176 | CDR2β | FYNNEI |
| 1177 | CDR3β | ASSGTLAGEVDTQY |
| 1178 | Vβ without signal | EPEVTQTPSHQVTQMGQEVILRCVPISNHLYFYWYRQI |
| | peptide (SignalP) | LGQKVEFLVSFYNNEISEKSEIFDDQFSVERPDGSNFT |
| | | LKIRSTKLEDSAMYFCASSGTLAGEVDTQYFGPGTRLT |
| | | VLEDLRN |
| 1179 | Vβ (without the | MDTWLVCWAIFSLLKAGLTEPEVTQTPSHQVTQMGQEV |
| | Constant) | ILRCVPISNHLYFYWYRQILGQKVEFLVSFYNNEISEK |
| | | SEIFDDQFSVERPDGSNFTLKIRSTKLEDSAMYFCASS |
| | | GTLAGEVDTQYFGPGTRLTVLEDLRN |
| 1180 | β chain with WT | MDTWLVCWAIFSLLKAGLTEPEVTQTPSHQVTQMGQEV |
| | signal peptide and | ILRCVPISNHLYFYWYRQILGQKVEFLVSFYNNEISEK |
| | constant Cβ | SEIFDDQFSVERPDGSNFTLKIRSTKLEDSAMYFCASS |
| | | GTLAGEVDTQYFGPGTRLTVLEDLRNEDLRNVTPPKVS |
| | | LFEPSKAEIANKQKATLVCLARGFFPDHVELSWWVNGK |
| | | EVHSGVSTDPQAYKESNYSYCLSSRLRVSATFWHNPRN |
| | | HFRCQVQFHGLSEEDKWPEGSPKPVTQNISAEAWGRAD |
| | | CGITSASYQQGVLSATILYEILLGKATLYAVLVSTLVV |
| | | MAMVKRKNS |

In some embodiments, CLL000032-TCR37 interacts with and/or is specific for a peptide from gene TP53. In some embodiments, the peptide is from a neoantigen of TP53 and has the amino acid change R175H (in which position 175 of the TP53 protein is mutated from Arg to His). In some embodiments, CLL000032-TCR37 interacts with and/or is specific for the neoantigen in the context of HLA-A*02:01.

TABLE 78

| SEQ ID NO. | Description | CLL000032-TCR385 |
|---|---|---|
| 1181 | CDR1α | TRDTTYY |
| 1182 | CDR2α | RNSFDEQN |
| 1183 | CDR3α | ALSFLNTGGFKTI |
| 1184 | Vα without signal | QKVTQAQTEISVVEKEDVTLDCVYETRDTTYYLFWYKQ |
| | peptide (SignalP) | PPSGELVFLIRRNSFDEQNEISGRYSWNFQKSTSSFNF |
| | | TITASQVVDSAVYFCALSFLNTGGFKTIFGAGTRLFVK |
| | | ANIQNPEPAV |
| 1185 | Vα only (without | MLTASLLRAVIASICVVSSMAQKVTQAQTEISVVEKED |
| | the Constant) | VTLDCVYETRDTTYYLFWYKQPPSGELVFLIRRNSFDE |
| | | QNEISGRYSWNFQKSTSSFNFTITASQVVDSAVYFCAL |
| | | SFLNTGGFKTIFGAGTRLFVKANIQNPEPAV |
| 1186 | α chain with WT | MLTASLLRAVIASICVVSSMAQKVTQAQTEISVVEKED |
| | signal peptide and | VTLDCVYETRDTTYYLFWYKQPPSGELVFLIRRNSFDE |
| | constant Cα | QNEISGRYSWNFQKSTSSFNFTITASQVVDSAVYFCAL |
| | | SFLNTGGFKTIFGAGTRLFVKANIQNPEPAVNIQNPEP |
| | | AVYQLKDPRSQDSTLCLFTDFDSQINVPKTMESGTFIT |
| | | DKTVLDMKAMDSKSNGAIAWSNQTSFTCQDIFKETNAT |
| | | YPSSDVPCDATLTEKSFETDMNLNFQNLLVIVLRILLL |
| | | KVAGFNLLMTLRLWSS |
| 1187 | CDR1β | SGHAT |
| 1188 | CDR2β | FQDESV |
| 1189 | CDR3β | ASSISGTGGLDTQY |
| 1190 | Vβ without signal | EVAQSPRYKITEKSQAVAFWCDPISGHATLYWYRQILG |
| | peptide (SignalP) | QGPELLVQFQDESVVDDSQLPKDRFSAERLKGVDSTLK |
| | | IQPAELGDSAMYLCASSISGTGGLDTQYFGPGTRLTVL |
| | | EDLRN |
| 1191 | Vβ (without the | MSTRLLCWMALCLLGAELSEAEVAQSPRYKITEKSQAV |
| | Constant) | AFWCDPISGHATLYWYRQILGQGPELLVQFQDESVVDD |
| | | SQLPKDRFSAERLKGVDSTLKIQPAELGDSAMYLCASS |
| | | ISGTGGLDTQYFGPGTRLTVLEDLRN |
| 1192 | β chain with WT | MSTRLLCWMALCLLGAELSEAEVAQSPRYKITEKSQAV |
| | signal peptide and | AFWCDPISGHATLYWYRQILGQGPELLVQFQDESVVDD |
| | constant Cβ | SQLPKDRFSAERLKGVDSTLKIQPAELGDSAMYLCASS |
| | | ISGTGGLDTQYFGPGTRLTVLEDLRNEDLRNVTPPKVS |
| | | LFEPSKAEIANKQKATLVCLARGFFPDHVELSWWVNGK |
| | | EVHSGVSTDPQAYKESNYSYCLSSRLRVSATFWHNPRN |
| | | HFRCQVQFHGLSEEDKWPEGSPKPVTQNISAEAWGRAD |
| | | CGITSASYQQGVLSATILYEILLGKATLYAVLVSTLVV |
| | | MAMVKRKNS |

In some embodiments, CLL000032-TCR385 interacts with and/or is specific for a peptide from gene KRAS or TP53. In some embodiments, the peptide is from a neoantigen of KRAS or TP53 and has the amino acid change G12D or R175H respectively (in which position 12 or 175 of the KRAS or TP53 protein is mutated from Gly or His to Asp or His). In some embodiments, CLL000032-TCR385 interacts with and/or is specific for the neoantigen in the context of DRA*01:01, DRB1*01:01, or DRB3*02:02.

TABLE 79

| SEQ ID NO. | Description | CLL000032-TCR421 |
|---|---|---|
| 1193 | CDR1α | TSDPSYG |
| 1194 | CDR2α | QGSYDQQN |
| 1195 | CDR3α | AMRDPGTGGFKTI |
| 1196 | Vα without signal | QKITQTQPGMFVQEKEAVTLDCTYDTSDPSYGLFWYKQ |
| | peptide (SignalP) | PSSGEMIFLIYQGSYDQQNATEGRYSLNFQKARKSANL |
| | | VISASQLGDSAMYFCAMRDPGTGGFKTIFGAGTRLFVK |
| | | ANIQNPEPAV |
| 1197 | Vα only (without | MSLSSLLKVVTASLWLGPGIAQKITQTQPGMFVQEKEA |
| | the Constant) | VTLDCTYDTSDPSYGLFWYKQPSSGEMIFLIYQGSYDQ |
| | | QNATEGRYSLNFQKARKSANLVISASQLGDSAMYFCAM |
| | | RDPGTGGFKTIFGAGTRLFVKANIQNPEPAV |
| 1198 | α chain with WT | MSLSSLLKVVTASLWLGPGIAQKITQTQPGMFVQEKEA |
| | signal peptide and | VTLDCTYDTSDPSYGLFWYKQPSSGEMIFLIYQGSYDQ |
| | constant Cα | QNATEGRYSLNFQKARKSANLVISASQLGDSAMYFCAM |
| | | RDPGTGGFKTIFGAGTRLFVKANIQNPEPAVNIQNPEP |
| | | AVYQLKDPRSQDSTLCLFTDFDSQINVPKTMESGTFIT |
| | | DKTVLDMKAMDSKSNGAIAWSNQTSFTCQDIFKETNAT |
| | | YPSSDVPCDATLTEKSFETDMNLNFQNLLVIVLRILLL |
| | | KVAGFNLLMTLRLWSS |
| 1199 | CDR1β | SGHTA |
| 1200 | CDR2β | FQGNSA |
| 1201 | CDR3β | ASSSMATGGVVGDTQY |
| 1202 | Vβ without signal | VSQSPSNKVTEKGKDVELRCDPISGHTALYWYRQRLGQ |
| | peptide (SignalP) | GLEFLIYFQGNSAPDKSGLPSDRFSAERTGESVSTLTI |
| | | QRTQQEDSAVYLCASSSMATGGVVGDTQYFGPGTRLTV |
| | | LEDLRN |
| 1203 | Vβ (without the | MGTRLLFWVAFCLLGAYHTGAGVSQSPSNKVTEKGKDV |
| | Constant) | ELRCDPISGHTALYWYRQRLGQGLEFLIYFQGNSAPDK |
| | | SGLPSDRFSAERTGESVSTLTIQRTQQEDSAVYLCASS |
| | | SMATGGVVGDTQYFGPGTRLTVLEDLRN |
| 1204 | β chain with WT | MGTRLLFWVAFCLLGAYHTGAGVSQSPSNKVTEKGKDV |
| | signal peptide and | ELRCDPISGHTALYWYRQRLGQGLEFLIYFQGNSAPDK |
| | constant Cβ | SGLPSDRFSAERTGESVSTLTIQRTQQEDSAVYLCASS |
| | | SMATGGVVGDTQYFGPGTRLTVLEDLRNEDLRNVTPPK |
| | | VSLFEPSKAEIANKQKATLVCLARGFFPDHVELSWWVN |
| | | GKEVHSGVSTDPQAYKESNYSYCLSSRLRVSATFWHNP |
| | | RNHFRCQVQFHGLSEEDKWPEGSPKPVTQNISAEAWGR |
| | | ADCGITSASYQQGVLSATILYEILLGKATLYAVLVSTL |
| | | VVMAMVKRKNS |

In some embodiments, CLL000032-TCR421 interacts with and/or is specific for a peptide from gene KRAS or TP53. In some embodiments, the peptide is from a neoantigen of KRAS or TP53 and has the amino acid change G12D or R175H respectively (in which position 12 or 175 of the KRAS or TP53 protein is mutated from Gly or His to Asp or His). In some embodiments, CLL000032-TCR421 interacts with and/or is specific for the neoantigen in the context of HLA-A*02:01, C*02:02, or C*07:27.

The present disclosure provides a polynucleotide encoding an amino acid sequence that is at least 90%, 91%, 92%, 93%, 94%, 95%, 96%, 97%, 98%, 99%, or 100% 10 identical to an amino acid sequence selected from the group consisting of SEQ ID NOs: 1-300, 536-1003, and 1025-1204 (the sequences provided in Tables 1-79).

In one aspect, the TCR used herein comprises a sequence selected from the TCR Cα or TCR Cβ provided in Tables 80 and 81.

TABLE 80
Amino acid sequences of TCR Cα regions.

| | | SEQ |
|---|---|---|
| | | ID |
| Description | Sequence | NO: |
| Cα (murine, | XIQNPEPAVYQLKDPRSQDSTLCLFTDFDSQINVPKTMESGTFI | 1004 |
| degenerate) | TDKXVLDMKAMDSKSNGAIAWSNQTSFTCQDIFKETNATYPSSD | |
| | VPCDATLTEKSFETDMNLNFQNLXVXXLRILLLKVAGFNLLMTL | |
| | RLWSS | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cα (murine, | NNNATCCAGAATCCCGAGCCTGCGGTGTACCAGCTGAAGGACCC | 1005 |
| degenerate) | CCGCTCTCAGGATAGCACACTGTGCCTGTTCACCGACTTTGATA | |
| (exemplary | GCCAGATCAACGTGCCTAAAACAATGGAGTCCGGCACCTTCATC | |
| nucleotide | ACCGACAAGNNNGTGCTGGATATGAAAGCGATGGACTCCAAGTC | |
| sequence) | TAACGGCGCGATCGCGTGGTCCAATCAGACATCTTTCACCTGCC | |
| | AGGATATCTTCAAGGAGACAAACGCGACCTATCCTTCCTCTGAC | |
| | GTGCCATGTGATGCGACACTGACCGAGAAGAGCTTCGAGACAGA | |
| | CATGAACCTGAATTTTCAGAATCTGNNNGTCNNNNNNCTGAGAA | |
| | TCCTGCTGCTGAAGGTGGCGGGCTTTAATCTGCTGATGACACTG | |
| | CGGCTGTGGAGTTCC | |
| | NNN at positions 1-3 make up a codon that encodes Asn, Asp, His, | |
| | or Tyr; | |
| | NNN at positions 142-144 make up a codon that encodes Thr or | |
| | Cys; | |
| | NNN at positions 334-336 make up a codon that encodes Ser, Ala, | |
| | Val, Leu, Ile, Pro, Phe, Met, or Trp; | |
| | NNN at positions 340-342 make up a codon that encodes Met, Ala, | |
| | Val, Leu, Ile, Pro, Phe, or Trp; | |
| | NNN at positions 343-345 make up a codon that encodes Gly, Ala, | |
| | Val, Leu, Ile, Pro, Phe, Met, or Trp | |
| Cα (murine, | NIQNPEPAVYQLKDPRSQDSTLCLFTDFDSQINVPKTMESGTFI | 1006 |
| cysteine- and | TDKCVLDMKAMDSKSNGAIAWSNQTSFTCQDIFKETNATYPSSD | |
| LIV- | VPCDATLTEKSFETDMNLNFQNLLVIVLRILLLKVAGFNLLMTL | |
| substituted) | RLWSS | |
| Cα (murine, | AACATCCAGAATCCCGAGCCTGCGGTGTACCAGCTGAAGGACCC | 1007 |
| cysteine- and | CCGCTCTCAGGATAGCACACTGTGCCTGTTCACCGACTTTGATA | |
| LIV- | GCCAGATCAACGTGCCTAAAACAATGGAGTCCGGCACCTTCATC | |
| substituted) | ACCGACAAGTGCGTGCTGGATATGAAAGCGATGGACTCCAAGTC | |
| (exemplary | TAACGGCGCGATCGCGTGGTCCAATCAGACATCTTTCACCTGCC | |
| nucleotide | AGGATATCTTCAAGGAGACAAACGCGACCTATCCTTCCTCTGAC | |
| sequence) | GTGCCATGTGATGCGACACTGACCGAGAAGAGCTTCGAGACAGA | |
| | CATGAACCTGAATTTTCAGAATCTGCTGGTCATCGTGCTGAGAA | |
| | TCCTGCTGCTGAAGGTGGCGGGCTTTAATCTGCTGATGACACTG | |
| | CGGCTGTGGAGTTCC | |
| Cα (murine, | NIQNPEPAVYQLKDPRSQDSTLCLFTDFDSQINVPKTMESGTFI | 1008 |
| LIV | TDKTVLDMKAMDSKSNGAIAWSNQTSFTCQDIFKETNATYPSSD | |
| substituted) | VPCDATLTEKSFETDMNLNFQNLLVIVLRILLLKVAGFNLLMTL | |
| | RLWSS | |
| Cα (murine, | AACATCCAGAATCCCGAGCCTGCGGTGTACCAGCTGAAGGACCC | 1009 |
| LIV | CCGCTCTCAGGATAGCACACTGTGCCTGTTCACCGACTTTGATA | |
| substituted) | GCCAGATCAACGTGCCTAAAACAATGGAGTCCGGCACCTTCATC | |
| (exemplary | ACCGACAAGACCGTGCTGGATATGAAAGCGATGGACTCCAAGTC | |
| nucleotide | TAACGGCGCGATCGCGTGGTCCAATCAGACATCTTTCACCTGCC | |
| sequence) | AGGATATCTTCAAGGAGACAAACGCGACCTATCCTTCCTCTGAC | |
| | GTGCCATGTGATGCGACACTGACCGAGAAGAGCTTCGAGACAGA | |
| | CATGAACCTGAATTTTCAGAATCTGCTGGTCATCGTGCTGAGAA | |
| | TCCTGCTGCTGAAGGTGGCGGGCTTTAATCTGCTGATGACACTG | |
| | CGGCTGTGGAGTTCC | |
| Cα (murine, | NIQNPEPAVYQLKDPRSQDSTLCLFTDFDSQINVPKTMESGTFI | 1010 |
| cysteine- | TDKCVLDMKAMDSKSNGAIAWSNQTSFTCQDIFKETNATYPSSD | |
| substituted) | VPCDATLTEKSFETDMNLNFQNLSVMGLRILLLKVAGFNLLMTL | |
| | RLWSS | |
| Cα (murine, | NIQNPEPAVYQLKDPRSQDSTLCLFTDFDSQINVPKTMESGTFI | 1011 |
| wild type) | TDKTVLDMKAMDSKSNGAIAWSNQTSFTCQDIFKETNATYPSSD | |
| | VPCDATLTEKSFETDMNLNFQNLSVMGLRILLLKVAGFNLLMTL | |
| | RLWSS | |
| Cα (human, | XIQNPDPAVYQLRDSKSSDKSVCLFTDFDSQTNVSQSKDSDVYI | 1012 |
| degenerate) | TDKXVLDMRSMDFKSNSAVAWSNKSDFACANAFNNSIIPEDTFF | |
| | PSPESSCDVKLVEKSFETDTNLNFQNLXVXXFRILLLKVAGFNL | |
| | LMTLRLWSS | |
| | X at position 1 is Asn, Asp, His, or Tyr X at position | |
| | 48 is Thr or Cys; X at position | |
| | 116 is Ser, Ala, Val, Leu, Ile, Pro, Phe, Met, or Trp; X at position | |
| | 118 is Met, Ala, Val, Leu, Ile, Pro, Phe, or Trp; X at position | |
| | 119 is Gly, Ala, Val, Leu, Ile, Pro, Phe, Met, or Trp | |
| Cα (human, | XIQNPDPAVYQLRDSKSSDKSVCLFTDFDSQTNVSQSKDSDVYI | 1013 |
| cysteine- and | TDKCVLDMRSMDFKSNSAVAWSNKSDFACANAENNSIIPEDTFF | |
| LIV- | PSPESSCDVKLVEKSFETDTNLNFQNLLVIVFRILLLKVAGFNL | |
| substituted; | LMTLRLWSS | |
| degenerate at | X at position 1 is Asn, Asp, His, or Tyr | |
| position 1) | | |
| Cα (human, | XIQNPDPAVYQLRDSKSSDKSVCLFTDFDSQTNVSQSKDSDVYI | 1014 |
| LIV- | TDKTVLDMRSMDFKSNSAVAWSNKSDFACANAENNSIIPEDTFF | |
| substituted; | PSPESSCDVKLVEKSFETDTNLNFQNLLVIVFRILLLKVAGFNL | |
| degenerate at | LMTLRLWSS | |
| position 1) | X at position 1 is Asn, Asp, His, or Tyr | |
| Cα (human, | XIQNPDPAVYQLRDSKSSDKSVCLFTDFDSQTNVSQSKDSDVYI | 1015 |
| cysteine- | TDKCVLDMRSMDFKSNSAVAWSNKSDFACANAFNNSIIPEDTFF | |
| substituted; | PSPESSCDVKLVEKSFETDTNLNFQNLSVIGFRILLLKVAGFNL | |
| degenerate at | LMTLRLWSS | |
| position 1) | X at position 1 is Asn, Asp, His, or Tyr | |
| Cα (human, | XIQNPDPAVYQLRDSKSSDKSVCLFTDFDSQTNVSQSKDSDVYI | 1016 |
| wild type; | TDKTVLDMRSMDFKSNSAVAWSNKSDFACANAENNSIIPEDTFF | |
| degenerate at | PSPESSCDVKLVEKSFETDINLNFQNLSVIGFRILLLKVAGFNL | |
| position 1) | LMTLRLWSS | |
| | X at position 1 is Asn, Asp, His, or Tyr | |

TABLE 81
Amino acid sequences of TCR Cβ regions.

| | | SEQ |
|---|---|---|
| | | ID |
| Description | Sequence | NO: |
| Cβ (murine, | EDLRNVTPPKVSLFEPSKAEIANKQKATLVCLARGFFPDHVELS | 1017 |
| degenerate) | WWVNGKEVHSGVXTDPQAYKESNYSYCLSSRLRVSATFWHNPRN | |
| | HFRCQVQFHGLSEEDKWPEGSPKPVTQNISAEAWGRADCGITSA | |
| | SYQQGVLSATILYEILLGKATLYAVLVSTLVVMAMVKRKNS | |
| | X at position 57 is Ser or Cys | |
| Cβ (murine, | GAGGACCTGAGGAACGTGACCCCACCTAAAGTGAGCCTGTTCGA | 1018 |
| degenerate) | GCCATCCAAGGCGGAGATCGCGAATAAGCAGAAAGCGACCCTGG | |
| (exemplary | TGTGCCTGGCGAGGGGCTTCTTTCCCGATCACGTGGAGCTGTCC | |
| nucleotide | TGGTGGGTGAACGGCAAAGAGGTGCACTCTGGCGTGNNNACAGA | |
| sequence) | CCCTCAGGCGTACAAGGAGAGCAATTACTCCTATTGTCTGTCTA | |
| | GCAGACTGAGGGTGAGCGCGACCTTTTGGCACAACCCCCGGAAT | |
| | CACTTCCGCTGCCAGGTGCAGTTTCACGGCCTGTCCGAGGAGGA | |
| | TAAATGGCCTGAGGGCTCTCCAAAGCCCGTGACACAGAATATCA | |
| | GCGCGGAGGCGTGGGGAAGAGCGGACTGTGGCATTACAAGCGCG | |
| | TCCTATCAGCAGGGCGTGCTGTCCGCGACCATCCTGTACGAGAT | |
| | TCTGCTGGGCAAGGCGACACTGTATGCGGTGCTGGTGTCCACCC | |
| | TGGTGGTCATGGCGATGGTGAAGAGGAAAAACTCT | |
| | NNN at positions 169-171 make up a codon that | |
| | encodes Ser or Cys | |
| Cβ (murine, | EDLRNVTPPKVSLFEPSKAEIANKQKATLVCLARGFFPDHVELS | 1019 |
| cysteine- | WWVNGKEVHSGVCTDPQAYKESNYSYCLSSRLRVSATFWHNPRN | |
| substituted) | HFRCQVQFHGLSEEDKWPEGSPKPVTQNISAEAWGRADCGITSA | |
| | SYQQGVLSATILYEILLGKATLYAVLVSTLVVMAMVKRKNS | |
| Cβ (murine, | GAGGACCTGAGGAACGTGACCCCACCTAAAGTGAGCCTGTTCGA | 1020 |
| cysteine- | GCCATCCAAGGCGGAGATCGCGAATAAGCAGAAAGCGACCCTGG | |
| substituted) | TGTGCCTGGCGAGGGGCTTCTTTCCCGATCACGTGGAGCTGTCC | |
| (exemplary | TGGTGGGTGAACGGCAAAGAGGTGCACTCTGGCGTGTGCACAGA | |
| nucleotide | CCCTCAGGCGTACAAGGAGAGCAATTACTCCTATTGTCTGTCTA | |
| sequence) | GCAGACTGAGGGTGAGCGCGACCTTTTGGCACAACCCCCGGAAT | |
| | CACTTCCGCTGCCAGGTGCAGTTTCACGGCCTGTCCGAGGAGGA | |
| | TAAATGGCCTGAGGGCTCTCCAAAGCCCGTGACACAGAATATCA | |
| | GCGCGGAGGCGTGGGGAAGAGCGGACTGTGGCATTACAAGCGCG | |
| | TCCTATCAGCAGGGCGTGCTGTCCGCGACCATCCTGTACGAGAT | |
| | TCTGCTGGGCAAGGCGACACTGTATGCGGTGCTGGTGTCCACCC | |
| | TGGTGGTCATGGCGATGGTGAAGAGGAAAAACTCT | |
| Cβ (murine, wild | EDLRNVTPPKVSLFEPSKAEIANKQKATLVCLARGFFPDHVELS | 1021 |
| type) | WWVNGKEVHSGVSTDPQAYKESNYSYCLSSRLRVSATFWHNPRN | |
| | HFRCQVQFHGLSEEDKWPEGSPKPVTQNISAEAWGRADCGITSA | |
| | SYQQGVLSATILYEILLGKATLYAVLVSTLVVMAMVKRKNS | |
| Cβ (human, | EDLKNVFPPEVAVFEPSEAEISHTQKATLVCLATGFYPDHVELS | 1022 |
| degenerate) | WWVNGKEVHSGVXTDPQPLKEQPALNDSRYCLSSRLRVSATFWQ | |
| | NPRNHFRCQVQFYGLSENDEWTQDRAKPVTQIVSAEAWGRADCG | |
| | FTSESYQQGVLSATILYEILLGKATLYAVLVSALVLMAMVKRKD | |
| | SRG | |
| | X at position 57 is Ser or Cys | |
| Cβ (human, | EDLKNVEPPEVAVFEPSEAEISHTQKATLVCLATGFYPDHVELS | 1023 |
| cysteine- | WWVNGKEVHSGVCTDPQPLKEQPALNDSRYCLSSRLRVSATFWQ | |
| substituted) | NPRNHFRCQVQFYGLSENDEWTQDRAKPVTQIVSAEAWGRADCG | |
| | FTSESYQQGVLSATILYEILLGKATLYAVLVSALVLMAMVKRKD | |
| | SRG | |
| Cβ (human, wild | EDLKNVFPPEVAVFEPSEAEISHTQKATLVCLATGFYPDHVELS | 1024 |
| type) | WWVNGKEVHSGVSTDPQPLKEQPALNDSRYCLSSRLRVSATFWQ | |
| | NPRNHFRCQVQFYGLSENDEWTQDRAKPVTQIVSAEAWGRADCG | |
| | FTSESYQQGVLSATILYEILLGKATLYAVLVSALVLMAMVKRKD | |
| | SRG | |

Non-limiting examples of HLA sequences and neoantigen peptide sequences are provided in Table 82 below. All the sequences are human.

TABLE 82

| SEQ ID | | | |
|---|---|---|---|
| NO: | Description | Sequence type | Sequence |
| SEQ ID | HLA-A*03:01 | Restricting HLA | MAVMAPRTLLLLLSGALALTQTWAGSHS |
| NO: 301 | | | MRYFFTSVSRPGRGEPRFIAVGYVDDTQ |
| | | | FVRFDSDAASQRMEPRAPWIEQEGPEYW |
| | | | DQETRNVKAQSQTDRVDLGTLRGYYNQS |
| | | | EAGSHTIQIMYGCDVGSDGRFLRGYRQD |
| | | | AYDGKDYIALNEDLRSWTAADMAAQITK |
| | | | RKWEAAHEAEQLRAYLDGTCVEWLRRYL |
| | | | ENGKETLQRTDPPKTHMTHHPISDHEAT |
| | | | LRCWALGFYPAEITLTWQRDGEDQTQDT |
| | | | ELVETRPAGDGTFQKWAAVVVPSGEEQR |
| | | | YTCHVQHEGLPKPLTLRWELSSQPTIPI |
| | | | VGIIAGLVLLGAVITGAVVAAVMWRRKS |
| | | | SDRKGGSYTQAASSDSAQGSDVSLTACK |
| | | | V |
| SEQ ID | HLA-B*35:02 | Restricting HLA | MRVTAPRTVLLLLWGAVALTETWAGSHS |
| NO: 302 | | | MRYFYTAMSRPGRGEPRFIAVGYVDDTQ |
| | | | FVRFDSDAASPRTEPRAPWIEQEGPEYW |
| | | | DRNTQIFKTNTQTYRESLRNLRGYYNQS |
| | | | EAGSHIIQRMYGCDLGPDGRFLRGHNQY |
| | | | AYDGKDYIALNEDLSSWTAADTAAQITQ |
| | | | RKWEAARVAEQLRAYLEGLCVEWLRRYL |
| | | | ENGKETLQRADPPKTHVTHHPVSDHEAT |
| | | | LRCWALGFYPAEITLTWQRDGEDQTQDT |
| | | | ELVETRPAGDRTFQKWAAVVVPSGEEQR |
| | | | YTCHVQHEGLPKPLTLRWEPSSQSTIPI |
| | | | VGIVAGLAVLAVVVIGAVVATVMCRRKS |
| | | | SGGKGGSYSQAASSDSAQGSDVSLTA |
| SEQ ID | HLA- | Restricting HLA | MRPEDRMFHIRAVILRALSLAFLLSLRG |
| NO: 303 | DPA1*01:03 | | AGAIKADHVSTYAAFVQTHRPTGEFMFE |
| | | | FDEDEMFYVDLDKKETVWHLEEFGQAFS |
| | | | FEAQGGLANIAILNNNLNTLIQRSNHTQ |
| | | | ATNDPPEVTVFPKEPVELGQPNTLICHI |
| | | | DKFFPPVLNVTWLCNGELVTEGVAESLF |
| | | | LPRTDYSFHKFHYLTFVPSAEDFYDCRV |
| | | | EHWGLDQPLLKHWEAQEPIQMPETTETV |
| | | | LCALGLVLGLVGIIVGTVLIIKSLRSGH |
| | | | DPRAQGTL |
| SEQ ID | HLA- | Restricting HLA | MRPEDRMFHIRAVILRALSLAFLLSLRG |
| NO: 304 | DPA1*03:01 | | AGAIKADHVSTYAMFVQTHRPTGEFMFE |
| | | | FDEDEMFYVDLDKKETVWHLEEFGQAFS |
| | | | FEAQGGLANIAISNNNLNTLIQRSNHTQ |
| | | | ATNDPPEVTVFPKEPVELGQPNTLICHI |
| | | | DKFFPPVLNVTWLCNGELVTEGVAESLF |
| | | | LPRTDYSFHKFHYLTFVPSAEDFYDCRV |
| | | | EHWGLDQPLLKHWEAQEPIQMPETTETV |
| | | | LCALGLVLGLVGIIVGTVLIIKSLRSGH |
| | | | DPRAQGTL |
| SEQ ID | HLA- | Restricting HLA | MMVLQVSAAPRTVALTALLMVLLTSVVQ |
| NO: 305 | DPB1*104:01 | | GRATPENYVYQLRQECYAFNGTQRFLER |
| | | | YIYNREEFVREDSDVGEFRAVTELGRPD |
| | | | EDYWNSQKDLLEEKRAVPDRVCRHNYEL |
| | | | DEAVTLQRRVQPKVNVSPSKKGPLQHHN |
| | | | LLVCHVTDFYPGSIQVRWFLNGQEETAG |
| | | | VVSTNLIRNGDWTFQILVMLEMTPQQGD |
| | | | VYICQVEHTSLDSPVTVEWKAQSDSARS |
| | | | KTLTGAGGFMLGLIICGVGIFMHRRSKK |
| | | | VQRGSA |
| SEQ ID | HLA- | Restricting HLA | MAISGVPVLGFFIIAVLMSAQESWAIKE |
| NO: 306 | DRA*01:01 | | EHVIIQAEFYLNPDQSGEFMFDEDGDEI |
| | | | FHVDMAKKETVWRLEEFGRFASFEAQGA |
| | | | LANIAVDKANLEIMTKRSNYTPITNVPP |
| | | | EVTVLTNSPVELREPNVLICFIDKFTPP |
| | | | VVNVTWLRNGKPVTTGVSETVFLPREDH |
| | | | LFRKFHYLPFLPSTEDVYDCRVEHWGLD |
| | | | EPLLKHWEFDAPSPLPETTENVVCALGL |
| | | | TVGLVGIIIGTIFIIKGVRKSNAAERRG |
| | | | PL |
| SEQ ID | HLA- | Restricting HLA | MVCLKLPGGSCMTALTVTLMVLSSPLAL |
| NO: 307 | DRB1*01:01 | | AGDTRPRFLWQLKFECHFENGTERVRLL |
| | | | ERCIYNQEESVREDSDVGEYRAVTELGR |
| | | | PDAEYWNSQKDLLEQRRAAVDTYCRHNY |
| | | | GVGESFTVQRRVEPKVTVYPSKTQPLQH |
| | | | HNLLVCSVSGFYPGSIEVRWERNGQEEK |
| | | | AGVVSTGLIQNGDWTFQTLVMLETVPRS |
| | | | GEVYTCQVEHPSVTSPLTVEWRARSESA |
| | | | QSKMLSGVGGFVLGLLFLGAGLFIYFRN |
| | | | QKGHSGLQPTGELS |
| SEQ ID | HLA- | Restricting HLA | MVCLRLPGGSCMAVLTVTLMVLSSPLAL |
| NO: 308 | DRB1*11:01 | | AGDTRPRFLEYSTSECHFENGTERVREL |
| | | | DRYFYNQEEYVREDSDVGEFRAVTELGR |
| | | | PDEEYWNSQKDFLEDRRAAVDTYCRHNY |
| | | | GVGESFTVQRRVHPKVTVYPSKTQPLQH |
| | | | HNLLVCSVSGFYPGSIEVRWERNGQEEK |
| | | | TGVVSTGLIHNGDWTFQTLVMLETVPRS |
| | | | GEVYTCQVEHPSVTSPLTVEWRARSESA |
| | | | QSKMLSGVGGFVLGLLFLGAGLFIYFRN |
| | | | QKGHSGLQPRGELS |
| SEQ ID | HLA- | Restricting HLA | MVCLKLPGGSCMAALTVTLTVLSSPLAL |
| NO: 309 | DRB4*01:03 | | AGDTQPRFLEQAKCECHFLNGTERVWNL |
| | | | IRYIYNQEEYARYNSDLGEYQAVTELGR |
| | | | PDAEYWNSQKDLLERRRAEVDTYCRYNY |
| | | | GVVESFTVQRRVQPKVTVYPSKTQPLQH |
| | | | HNLLVCSVNGFYPGSIEVRWFRNGQEEK |
| | | | AGVVSTGLIQNGDWTFQTLVMLETVPRS |
| | | | GEVYTCQVEHPSMMSPLTVQWSARSESA |
| | | | QSKMLSGVGGFVLGLLFLGTGLFIYFRN |
| | | | QKGHSGLQPTGLLS |
| SEQ ID | 2599_TWNK_p. | Mutation/Peptide | RRRYRKETLQALYMPVLPVTATEIR |
| NO: 310 | D49Y | | |
| SEQ ID | 2599_SOX6_ | Mutation/Peptide | TSREKEEGSDQHEASHLPLHPIMHN |
| NO: 311 | p.V37E | | |
| SEQ ID | 2599_NAA40_ | Mutation/Peptide | VLYCYEVQLESKLRRKGLGKFLIQI |
| NO: 312 | p.V146L | | |
| SEQ ID | 2599_ATG2A_ | Mutation/Peptide | TTNLGGPECCLRISLMPLRLNVDQD |
| NO: 313 | p.V1580I | | |
| SEQ ID | 2599_SLCO1B3_ | Mutation/Peptide | SHMWIYVEMGNMRRGIGETPIVPLG |
| NO: 314 | p.L180R | | |
| SEQ ID | 2599_ERGIC2_ | Mutation/Peptide | SQSPNACRIHGHPYVNKVAGNFHIT |
| NO: 315 | p.L176P | | |
| SEQ ID | 2599_CSAD_ | Mutation/Peptide | PSARRSYLCTLPLALLSREILMADS |
| NO: 316 | p.P88L | | |
| SEQ ID | 2599_CSAD_ | Mutation/Peptide | SSFLLSYLCTLPLALLSREILMADS |
| NO: 317 | p.P19L | | |
| SEQ ID | 2599_NAV3_ | Mutation/Peptide | EEKAHSEQIHKLWRELVASQEKVAT |
| NO: 318 | p.R1575W | | |
| SEQ ID | 2599_ABHD13_ | Mutation/Peptide | LIALASWSWALCCISLLPLIVTFHL |
| NO: 319 | p.R27C | | |
| SEQ ID | 2599_NBEA_ | Mutation/Peptide | VNDVDLPPWAKKTEDEVRINRMALE |
| NO: 320 | p.P2474T | | |
| SEQ ID | 2599_THSD1_ | Mutation/Peptide | HVGSRGGPSERSRARNAHERRTASF |
| NO: 321 | p.H644R | | |
| SEQ ID | 2599_GPC6_ | Mutation/Peptide | FENLVEETSHFVCTTFVSRHKKEDE |
| NO: 322 | p.R95C | | |
| SEQ ID | 2599_CERS3_ | Mutation/Peptide | EMSFYWSLLFRLVFDVKRKDFLAHI |
| NO: 323 | p.G197V | | |
| SEQ ID | 2599_GLDN_ | Mutation/Peptide | GEKGDKGDVSNDMLLAGAKGDQGPP |
| NO: 324 | p.V226M | | |
| SEQ ID | 2599_FAM219B_ | Mutation/Peptide | DEDLDLIPPKPMVSSTCSCCWCCLG |
| NO: 325 | p.A178V | | |
| SEQ ID | 2599_BAIAP3_ | Mutation/Peptide | ELSTPAATILCLLGAQSNLSPLQLA |
| NO: 326 | p.H425L | | |
| SEQ ID | 2599_CLEC19A_ | Mutation/Peptide | LFWMEFKGHCYRLFPLNKTWAEADL |
| NO: 327 | p.F54L | | |
| SEQ ID | 2599_ZPBP2_ | Mutation/Peptide | RSCIGRYNDVFFGVLKKILDSLISD |
| NO: 328 | p.R165G | | |
| SEQ ID | 2599_ASB16_ | Mutation/Peptide | APLAIATARGYTGCARHLIRQGAEL |
| NO: 329 | p.D125G | | |
| SEQ ID | 2599_ARHGAP27_ | Mutation/Peptide | SESSRVDFGSSEHLGSWQEKEEDAR |
| NO: 330 | p.R635H | | |
| SEQ ID | 2599_TACO1_ | Mutation/Peptide | RHIKGPKDVERSPIFSKLCLNIRLA |
| NO: 331 | p.R79P | | |
| SEQ ID | 2599_TP53_ | Mutation/Peptide | SGNLLGRNSFEVCVCACPGRDRRTE |
| NO: 332 | p.R273C | | |
| SEQ ID | 2599_PLEKHG2_ | Mutation/Peptide | GSWSSAPTSRASLPPPQPQPPPPPA |
| NO: 333 | p.S1318L | | |
| SEQ ID | 2599_ZNF229_ | Mutation/Peptide | HQKTHTGERPYQWDKCGKGFSHNSY |
| NO: 334 | p.C491W | | |
| SEQ ID | 2599_TRIM33_ | Mutation/Peptide | HSQHYQIPDDFVPDVRLIFKNCERF |
| NO: 335 | p.A1026P | | |
| SEQ ID | 2599_MLLT11_ | Mutation/Peptide | LFWRMPIPELDLLELEGLGLSDTAT |
| NO: 336 | p.S25L | | |
| SEQ ID | 2599_SMCP_ | Mutation/Peptide | QNQCCQSKGNQCYPPKQNQCCQPKG |
| NO: 337 | p.C35Y | | |
| SEQ ID | 2599_LRRN2_ | Mutation/Peptide | ITNNPRLSFIHPHAFHHLPQMETLM |
| NO: 338 | p.R329H | | |
| SEQ ID | 2599_HHIPL2_ | Mutation/Peptide | RGLQESHGRDGTHFCHLLDLPDKDY |
| NO: 339 | p.R167H | | |
| SEQ ID | 2599_RYR2_ | Mutation/Peptide | VLNYFQPFLGRIKIMGSAKRIERVY |
| NO: 340 | p.E4156K | | |
| SEQ ID | 2599_OR2M4_ | Mutation/Peptide | TSAFERLLVICCAVMLIFPVSVIIL |
| NO: 341 | p.V204A | | |
| SEQ ID | 2599_OR2T3_ | Mutation/Peptide | ILHLIHRMNSAASHRKALATCSSHM |
| NO: 342 | p.G238S | | |
| SEQ ID | 2599_LEPROT_ | Mutation/Peptide | LAGNAVIFLTIQRFFLIFGRGDDES |
| NO: 343 | p.G124R | | |
| SEQ ID | 2599_FPGT_ | Mutation/Peptide | LTKAALPAHSFVGSLSLKMNRCLKY |
| NO: 344 | p.C467G | | |
| SEQ ID | 2599_NAPB_ | Mutation/Peptide | EMFPAFTDSRECQLLKKLLEAHEEQ |
| NO: 345 | p.K246Q | | |
| SEQ ID | 2599_ITCH_ | Mutation/Peptide | QRSQLQGAMQQFKQRFIYGNQDLFA |
| NO: 346 | p.N415K | | |
| SEQ ID | 2599_LONRF2_ | Mutation/Peptide | CPRCRRLLHKPVMLPCGLTVCKRCV |
| NO: 347 | p.T155M | | |
| SEQ ID | 2599_IL1RL1_ | Mutation/Peptide | TRNDGKLYDAYVFYPRNYKSSTDGA |
| NO: 348 | p.V382F | | |
| SEQ ID | 2599_AMER3_ | Mutation/Peptide | ASAPECRCSLLACEGLLCGQPEVGA |
| NO: 349 | p.R839C | | |
| SEQ ID | 2599_LCT_ | Mutation/Peptide | IEGAWRADGKGLIIWDTFSHTPLRV |
| NO: 350 | p.S1404I | | |
| SEQ ID | 2599_DPP4_ | Mutation/Peptide | NIIVASFDGRGSVYQGDKIMHAINR |
| NO: 351 | p.G584V | | |
| SEQ ID | 2599_TLK1_ | Mutation/Peptide | EDIERQRKLLAKCKPPTANNSQAPS |
| NO: 352 | p.R370C | | |
| SEQ ID | 2599_FSIP2_ | Mutation/Peptide | QMFSVSEISTVAHEITDSVLNILHK |
| NO: 353 | p.Q1257H | | |
| SEQ ID | 2599_CFAP44_ | Mutation/Peptide | KQLIREKREMTKNIHKMEETVRQLM |
| NO: 354 | p.T1734N | | |
| SEQ ID | 2599_CLSTN2_ | Mutation/Peptide | AGLLVDSSEMIFNEDGRQGAKVPDG |
| NO: 355 | p.K357N | | |
| SEQ ID | 2599_FGD5_ | Mutation/Peptide | SAQRWIEAMEDATVL |
| NO: 356 | p.S1503T | | |
| SEQ ID | 2599_LRRC2_ | Mutation/Peptide | MESERDRQHFDKVVMKAYIEDLKER |
| NO: 357 | p.E343V | | |
| SEQ ID | 2599_PDZRN3_ | Mutation/Peptide | AQLELQMTALRYRKKFTEYSARLDS |
| NO: 358 | p.Q217R | | |
| SEQ ID | 2599_CRYBG3_ | Mutation/Peptide | FQEHFGIYTGKIFIDFPTAAQFDNL |
| NO: 359 | p.S1137F | | |
| SEQ ID | 2599_FAT4_ | Mutation/Peptide | PAIVGSCATVLAFLVLSLILCNQCR |
| NO: 360 | p.L4520F | | |
| SEQ ID | 2599_CCDC149_ | Mutation/Peptide | KGIPEGGGMRSTMKT |
| NO: 361 | p.V527M | | |
| SEQ ID | 2599_EVC_ | Mutation/Peptide | LSRTFLRVNAFPGVLACESVDVDLC |
| NO: 362 | p.E200G | | |
| SEQ ID | 2599_NPFFR2_ | Mutation/Peptide | VMEELKETTNSSKI |
| NO: 363 | p.E521K | | |
| SEQ ID | 2599_KLHL3_ | Mutation/Peptide | DQWTSIASMQERQSTLGAAVLNDLL |
| NO: 364 | p.R352Q | | |
| SEQ ID | 2599_PCDHA9_ | Mutation/Peptide | QLTIKTLSVPVKKDAQLGTVIALIS |
| NO: 365 | p.E361K | | |
| SEQ ID | 2599_PCDHA9_ | Mutation/Peptide | ESVSAYELVVTAQDRGSPSLWATAR |
| NO: 366 | p.R428Q | | |
| SEQ ID | 2599_GRIA1_ | Mutation/Peptide | VNLAVLKLSEQGILDKLKSKWWYDK |
| NO: 367 | p.V782I | | |
| SEQ ID | 2599_RXFP3_ | Mutation/Peptide | GVVVYSGGRYDLMPSSSAY |
| NO: 368 | p.L463M | | |
| SEQ ID | 2599_GDNF_ | Mutation/Peptide | AANMPEDYPDQFHDVMDFIQATIKR |
| NO: 369 | p.D53H | | |
| SEQ ID | 2599_GDNF_ | Mutation/Peptide | DSNMPEDYPDQFHDVMDFIQATIKR |
| NO: 370 | p.D79H | | |
| SEQ ID | 2599_STX11_ | Mutation/Peptide | QHGPHSAVARISWAQYNALTLTFQR |
| NO: 371 | p.R129W | | |
| SEQ ID | 2599_OR2B3_ | Mutation/Peptide | YFFLTNLSILDLYYTTTTVPHMLVN |
| NO: 372 | p.C72Y | | |
| SEQ ID | 2599_THSD7A_ | Mutation/Peptide | GSSRTVWCQRSDCINVTGGCLVMSQ |
| NO: 373 | p.G1498C | | |
| SEQ ID | 2599_FLNC_ | Mutation/Peptide | KHTIIISWGGVNMPKSPFRVNVGEG |
| NO: 374 | p.V749M | | |
| SEQ ID | 2599_SVOPL_ | Mutation/Peptide | ERDLVCGSKSDSGVVVTGGDSGESQ |
| NO: 375 | p.A319G | | |
| SEQ ID | 2599_SLC37A3_ | Mutation/Peptide | AKETGSHIEGVTSARETERTMSATS |
| NO: 376 | p.G396S | | |
| SEQ ID | 2599_DNAH11_ | Mutation/Peptide | YALRNFVEEKLGVKYVERTRLDLVK |
| NO: 377 | p.A3906V | | |
| SEQ ID | 2599_CRHR2_ | Mutation/Peptide | ILMTKLRASTTSKTIQYRKAVKATL |
| NO: 378 | p.E301K | | |
| SEQ ID | 2599_TECPR1_ | Mutation/Peptide | PSPQAIWSITCKEDIFVSEPSPDLE |
| NO: 379 | p.G738E | | |
| SEQ ID | 2599_DCAF13_ | Mutation/Peptide | SRNPDNYVRETKFDLQRVPRNYDPA |
| NO: 380 | p.L171F | | |
| SEQ ID | 2599_TMEM2_ | Mutation/Peptide | LGVLEQFIPLQLHEYGCPRATTVRR |
| NO: 381 | p.D1358H | | |
| SEQ ID | 2599_MAGEC1_ | Mutation/Peptide | PHYFPQSPPQGEGSLSPHYFPQSPQ |
| NO: 382 | p.D573G | | |
| SEQ ID | 2599_PHEX_ | Mutation/Peptide | LGYIKKVIDTRLFPHLKDISPSENV |
| NO: 383 | p.Y327F | | |
| SEQ ID | 2599_PCDH19_ | Mutation/Peptide | NPMPIRSKSPEHMRNIIALSIEATA |
| NO: 384 | p.V987M | | |
| SEQ ID | 2599_ERGIC2_ | Mutation/Peptide | RIHGHPYVNK |
| NO: 385 | p.L176P* | | |
| SEQ ID | 8434_ANXA9_ | Mutation/Peptide | ALLGLASVIKNTSLYFADKLHQALQ |
| NO: 386 | p.P272S | | |
| SEQ ID | 8434_ANXA9_ | Mutation/Peptide | GSARPSFGDQEHIAVLC |
| NO: 387 | p.T268I | | |
| SEQ ID | 8434_DCHS2_ | Mutation/Peptide | LIPGNVSSLFTIESTTGIIYLTLPL |
| NO: 388 | p.D822-1 | | |
| SEQ ID | 8434_PRRC2C_ | Mutation/Peptide | LASAPLPPSTLASVSASASVSASVP |
| NO: 389 | p.P1763S | | |
| SEQ ID | 8434_CRB1_ | Mutation/Peptide | AEKEPEFLNISIHDSRLFFQLQSGN |
| NO: 390 | p.Q980H | | |
| SEQ ID | 8434_TRIM67_ | Mutation/Peptide | PEHEMENYSMYCMSCRTPVCYLCLE |
| NO: 391 | p.V316M | | |
| SEQ ID | 8434_RYR2_ | Mutation/Peptide | HKNPVPQCPPRLRVQFLSHVLWSRM |
| NO: 392 | p.H1587R | | |
| SEQ ID | 8434_SPOCD1_ | Mutation/Peptide | SCRLVQALPTVICSAGCIPSNIVWD |
| NO: 393 | p.R902C | | |
| SEQ ID | 8434_ARHGEF16_ | Mutation/Peptide | DKDPGGMLRRNLWNQSYRAAMKGLG |
| NO: 394 | p.R150W | | |
| SEQ ID | 8434_CSMD2_ | Mutation/Peptide | HCVWLILARPESHIHLAFNDIDVEP |
| NO: 395 | p.R655H | | |
| SEQ ID | 8434_ZCCHC11_ | Mutation/Peptide | SLPPPSPAHLAAFSVAVIELAKEHG |
| NO: 396 | p.L355F | | |
| SEQ ID | 8434_SGMS1_ | Mutation/Peptide | FCIVGTLYLYRCLTMYVTTLPVPGM |
| NO: 397 | p.I228L | | |
| SEQ ID | 8434_DYNC2H1_ | Mutation/Peptide | HKEWIVIGQVDMAALVEKHLFTVHD |
| NO: 398 | p.E869A | | |
| SEQ ID | 8434_CWF19L2_ | Mutation/Peptide | TITQIPKKSGVEYEDQQEVILVRTD |
| NO: 399 | p.N540Y | | |
| SEQ ID | 8434_ESAM_ | Mutation/Peptide | VSLVYSMPSRNLYLRLEGLQEKDSG |
| NO: 400 | p.S110Y | | |
| SEQ ID | 8434_METTL15_ | Mutation/Peptide | WLESGIPNLGVWAKRIHTTAEKYRE |
| NO: 401 | p.P30A | | |
| SEQ ID | 8434_LRP4_ | Mutation/Peptide | ETVIGRGLKTTDRLAVDWVARNLYW |
| NO: 402 | p.G1432R | | |
| SEQ ID | 8434_FOLH1_ | Mutation/Peptide | VQAAAETLSEVAQ |
| NO: 403 | p.L720Q | | |
| SEQ ID | 8434_UTP20_ | Mutation/Peptide | VRGYQVHVLTFTIHMLLQGLINKLQ |
| NO: 404 | p.V1905I | | |
| SEQ ID | 8434_KRAS_ | Mutation/Peptide | ETCLLDILDTAGHEEYSAMRDQYMR |
| NO: 405 | p.Q61H | | |
| SEQ ID | 8434_PARP4_ | Mutation/Peptide | FSDSLSTSIKYSQPGETDGTRLLLI |
| NO: 406 | p.H490Q | | |
| SEQ ID | 8434_NPAS3_ | Mutation/Peptide | KVERYVESESDLLLQNCESLTSDSA |
| NO: 407 | p.R568L | | |
| SEQ ID | 8434_BAZ1A_ | Mutation/Peptide | TKDLTEALDEDANPTKSALSAVASL |
| NO: 408 | p.D514N | | |
| SEQ ID | 8434_FANCM_ | Mutation/Peptide | LNSKSESLPVSDNTAISETPLVSQF |
| NO: 409 | p.K1138N | | |
| SEQ ID | 8434_ASB2_ | Mutation/Peptide | PPAPQPSSRENDVPAADKEPSVVQF |
| NO: 410 | p.A528V | | |
| SEQ ID | 8434_RYR3_ | Mutation/Peptide | VYLYTVVAFNFFCKFYNKSEDDDEP |
| NO: 411 | p.R4693C | | |
| SEQ ID | 8434_C15orf41_ | Mutation/Peptide | SIFSQEYQKHIKTTHAKHHTSEAIE |
| NO: 412 | p.R54T | | |
| SEQ ID | 8434_FEM1B_ | Mutation/Peptide | NRVKNISDADVHTAMDNYECNLYTF |
| NO: 413 | p.N435T | | |
| SEQ ID | 8434_PRR35_ | Mutation/Peptide | PKASPSLTRFCSQSSLPTGSSVMLW |
| NO: 414 | p.R359Q | | |
| SEQ ID | 8434_BEAN1_ | Mutation/Peptide | SFKRPCPLARYNHTSYFYPTFSESS |
| NO: 415 | p.R14H | | |
| SEQ ID | 8434_CES2_ | Mutation/Peptide | QFWKKALPQKIQKLEEPEERHTEL |
| NO: 416 | p.E612K | | |
| SEQ ID | 8434_DHX38_ | Mutation/Peptide | DILFSKTPQEDYMEAAVKQSLQVHL |
| NO: 417 | p.V724M | | |
| SEQ ID | 8434_STUB1_ | Mutation/Peptide | ERRIHQESELHSCLSRLIAAERERE |
| NO: 418 | p.Y164C | | |
| SEQ ID | 8434_GRIN2A_ | Mutation/Peptide | TCVRNTVPCRKFIKINNSTNEGMNV |
| NO: 419 | p.V440I | | |
| SEQ ID | 8434_HS3ST3B1_ | Mutation/Peptide | GTRALLEFLRVHSDVRAVGAEPHFF |
| NO: 420 | p.P161S | | |
| SEQ ID | 8434_TMEM132E_ | Mutation/Peptide | VFLINCIVFVLRCRHKRIPPEGQTS |
| NO: 421 | p.Y916C | | |
| SEQ ID | 8434_TP53_ | Mutation/Peptide | YMCNSSCMGGMNQRPILTIITLEDS |
| NO: 422 | p.R248Q | | |
| SEQ ID | 8434_SALL3_ | Mutation/Peptide | AATDPAKPLLSYEGSCPPSPPSVIS |
| NO: 423 | p.A826E | | |
| SEQ ID | 8434_ZNF443_ | Mutation/Peptide | CKECGKSFSSLGILQRHMAVQRGDG |
| NO: 424 | p.N185I | | |
| SEQ ID | 8434_CACNA1A_ | Mutation/Peptide | NGYYPAHGLARPHGPGSRKGLHEPY |
| NO: 425 | p.R2486H | | |
| SEQ ID | 8434_BABAM1_ | Mutation/Peptide | ALELHNCMAKLLSHPLQRPCQSHAS |
| NO: 426 | p.A300S | | |
| SEQ ID | 8434_NPHS1_ | Mutation/Peptide | ESRRVHLGSVEKYGSTFSRELVLVT |
| NO: 427 | p.S505Y | | |
| SEQ ID | 8434_PNMAL2_ | Mutation/Peptide | QPDLPPQAKKAGCGLEGGWSEHRED |
| NO: 428 | p.R432C | | |
| SEQ ID | 8434_SBK2_ | Mutation/Peptide | FLYEFCVGLSLGTHSAIVTAYGIGI |
| NO: 429 | p.A115T | | |
| SEQ ID | 8434_ZNF865_ | Mutation/Peptide | KSFNRRESLKRHAKTHSADLLRLPC |
| NO: 430 | p.V397A | | |
| SEQ ID | 8434_REV1_ | Mutation/Peptide | KNPLLHLKAAVKGKKRNKKKKTIGS |
| NO: 431 | p.E1069G | | |
| SEQ ID | 8434_OLA1_ | Mutation/Peptide | QGRNYIVEDGDINFFKENTPQQPKK |
| NO: 432 | p.I225N | | |
| SEQ ID | 8434_ABCA12_ | Mutation/Peptide | SQTTLEEVFINFSKDQKSYETADTS |
| NO: 433 | p.A2248S | | |
| SEQ ID | 8434_EPAS1_ | Mutation/Peptide | RFPPQCYATQYQNYSLSSAHKVSGM |
| NO: 434 | p.D810N | | |
| SEQ ID | 8434_DOPEY2_ | Mutation/Peptide | LYLPLIQERLTDILRVGQTSIVAAQ |
| NO: 435 | p.N2059I | | |
| SEQ ID | 8434_TXNRD2_ | Mutation/Peptide | ACLPTTVGHAGKKQRRD |
| NO: 436 | p.N334K | | |
| SEQ ID | 8434_CELSR1_ | Mutation/Peptide | PSEDLQEQIYLNWTLLTTISTQRVL |
| NO: 437 | p.R1288W | | |
| SEQ ID | 8434_MYLK_ | Mutation/Peptide | CASDIRSSSLTLTWYGSSYDGGSAV |
| NO: 438 | p.S1175T | | |
| SEQ ID | 8434_KCNAB1_ | Mutation/Peptide | ISEENTKLRRQSSFSVAGKDKSPKK |
| NO: 439 | p.G25S | | |
| SEQ ID | 8434_SI_ | Mutation/Peptide | MARKKFCGLEISLIVLEVI |
| NO: 440 | p.S7C | | |
| SEQ ID | 8434_RAB5A_ | Mutation/Peptide | GNKICQFKLVLLEESAVGKSSLVLR |
| NO: 441 | p.G27E | | |
| SEQ ID | 8434_CSPG5_ | Mutation/Peptide | SAALVLLLLFMMMVFFAKKLYLLKT |
| NO: 442 | p.T440M | | |
| SEQ ID | 8434_BSN_ | Mutation/Peptide | RPLKSAEEAYEELMRKAELLQRQQG |
| NO: 443 | p.M1191L | | |
| SEQ ID | 8434_ITIH3_ | Mutation/Peptide | SQKDYRKDASIGMKVVCWFVHNNGE |
| NO: 444 | p.T862M | | |
| SEQ ID | 8434_OR5H14_ | Mutation/Peptide | LYPAIMTNGLCIQLLILSYVGGLLH |
| NO: 445 | p.R143Q | | |
| SEQ ID | 8434_TRPC3_ | Mutation/Peptide | NSKSRLNLFTQSISRVFESHSENSI |
| NO: 446 | p.N829I | | |
| SEQ ID | 8434_FBXW7_ | Mutation/Peptide | VETGNCIHTLTGQQSLTSGMELKDN |
| NO: 447 | p.H580Q | | |
| SEQ ID | 8434_NSD2_ | Mutation/Peptide | PPPEPGKPKGKRWRRRGWRRVTEGK |
| NO: 448 | p.R1353W | | |
| SEQ ID | 8434_MYO10_ | Mutation/Peptide | SREDTDDELSYRHDSVYSCVTLPYF |
| NO: 449 | p.R1166H | | |
| SEQ ID | 8434_ROS1_ | Mutation/Peptide | ERMHFIHRDLAASNCLVSVKDYTSP |
| NO: 450 | p.R2083S | | |
| SEQ ID | 8434_MCM9_ | Mutation/Peptide | AHLTCEGDKKEEASGSNKSGKVHAC |
| NO: 451 | p.V1041A | | |
| SEQ ID | 8434_FNDC1_ | Mutation/Peptide | ATLRAPRRLSWAVLLLLAALLPVAS |
| NO: 452 | p.A19V | | |
| SEQ ID | 8434_RGL2_ | Mutation/Peptide | GSPLSGGAEEASEGTGYGGEGSGPG |
| NO: 453 | p.G551E | | |
| SEQ ID | 8434_TOP1MT_ | Mutation/Peptide | FIDKLALRAGNEEEDGEAADTVGCC |
| NO: 454 | p.K231E | | |
| SEQ ID | 8434_TMEM55A_ | Mutation/Peptide | KCTVCNEATPIKTPPTGKKYVRCPC |
| NO: 455 | p.N89T | | |
| SEQ ID | 8434_COL4A5_ | Mutation/Peptide | GDQGLPGDRGPPEPPGIRGPPGPPG |
| NO: 456 | p.G270E | | |
| SEQ ID | 8434_ATP11C_ | Mutation/Peptide | SARNPNLELPMLFSYKHTDSGYS |
| NO: 457 | p.L1109F | | |
| SEQ ID | 8434_DCHS2_ | Mutation/Peptide | LIPGNVSSLFTIESTTGLYSPEVEI |
| NO: 458 | p.D228E-2 | | |
| SEQ ID | 8434_PRRC2C_ | Mutation/Peptide | STSAPVPASPLASVSASASVSASVP |
| NO: 459 | p.P1808S | | |
| SEQ ID | 6932_KDELC2_ | Mutation/Peptide | NHVYRRSLGKYTGFKMESDEILLSL |
| NO: 460 | p.D213G | | |
| SEQ ID | 6932_SLC5A12_ | Mutation/Peptide | QENLENGSARKQEAESVLQNGLRRE |
| NO: 461 | p.G581E | | |
| SEQ ID | 6932_CMKLR1_ | Mutation/Peptide | KISCFNNESLSTSGSSSWPTHSQMD |
| NO: 462 | p.P198S | | |
| SEQ ID | 6932_RIMBP2_ | Mutation/Peptide | ARCRSESDMENEQNSNTSKQRYSGK |
| NO: 463 | p.R173Q | | |
| SEQ ID | 6932_AKAP3_ | Mutation/Peptide | LAQGGRRDARSFIEAAGTTNFPANE |
| NO: 464 | p.V551I | | |
| SEQ ID | 6932_ATP11A_ | Mutation/Peptide | VLKRDPTLYRDVTKNALLRWRVFIY |
| NO: 465 | p.A955T | | |
| SEQ ID | 6932_NDFIP2_ | Mutation/Peptide | GGRGPAATTSSTAVAVGAEHGEDSL |
| NO: 466 | p.G72A | | |
| SEQ ID | 6932_CSK_ | Mutation/Peptide | LFLVRESTNYPGYYTLCVSCDGKVE |
| NO: 467 | p.D115Y | | |
| SEQ ID | 6932_IGF1R_ | Mutation/Peptide | RCQKMCPSTCGKQACTENNECCHPE |
| NO: 468 | p.R222Q | | |
| SEQ ID | 6932_RHBDL1_ | Mutation/Peptide | KRAIANGQRALPWDGPLDEPGLGVY |
| NO: 469 | p.R154W | | |
| SEQ ID | 6932_HGS_ | Mutation/Peptide | QIMKVEGHVFPEIKESDAMFAAERA |
| NO: 470 | p.F145I | | |
| SEQ ID | 6932_MAPK4_ | Mutation/Peptide | VDGGASPQFDLDEFISRALKLCTKP |
| NO: 471 | p.V541E | | |
| SEQ ID | 6932_SERPINB4_ | Mutation/Peptide | VEAAAATAVVVVKLSSPSTNEEFCC |
| NO: 472 | p.E353K | | |
| SEQ ID | 6932_BTBD2_ | Mutation/Peptide | VFDAMENGGMATKSTEIELPDVEPA |
| NO: 473 | p.T157K | | |
| SEQ ID | 6932_ZNF114_ | Mutation/Peptide | AFREDGSLRAHNAHGREKMYDFTQC |
| NO: 474 | p.T241A | | |
| SEQ ID | 6932_MCOLN1_ | Mutation/Peptide | ESELQAYIAQCQHSPTSGKFRRGSG |
| NO: 475 | p.D546H | | |
| SEQ ID | 6932_RYR2_ | Mutation/Peptide | NPVEGERYLDFLSFAVFCNGESVEE |
| NO: 476 | p.R2303S | | |
| SEQ ID | 6932_OR2T10_ | Mutation/Peptide | VGSVDGFMLTPISMSFPFCRSHEIQ |
| NO: 477 | p.A163S | | |
| SEQ ID | 6932_CC2D1B_ | Mutation/Peptide | KLQYQRAALQAKHSQDLEQAKAYLR |
| NO: 478 | p.R550H | | |
| SEQ ID | 6932_ZZZ3_ | Mutation/Peptide | AHPEEISSNSQVLSRSPKKRPEPVP |
| NO: 479 | p.R46L | | |
| SEQ ID | 6932_THBD_ | Mutation/Peptide | LVVALLALLCHLCKKQGAARAKMEY |
| NO: 480 | p.R540C | | |
| SEQ ID | 6932_HELZ2_ | Mutation/Peptide | NPIHARGKVPPHARHYPLMFCHVAG |
| NO: 481 | p.P775A | | |
| SEQ ID | 6932_EVX2_ | Mutation/Peptide | GAAQLKENNGKGFAESGSAAGTTTS |
| NO: 482 | p.Y144F | | |
| SEQ ID | 6932_D2HGDH_ | Mutation/Peptide | SVSGILVCQAGCILEELSRYVEERD |
| NO: 483 | p.V173I | | |
| SEQ ID | 6932_CD207_ | Mutation/Peptide | GPSLVPGKTPTVCAALICLTLVLVA |
| NO: 484 | p.R43C | | |
| SEQ ID | 6932_PIK3CA_ | Mutation/Peptide | ALEYFMKQMNDARHGGWTTKMDWIF |
| NO: 485 | p.H1047R | | |
| SEQ ID | 6932_MTMR14_ | Mutation/Peptide | GAIGGLLEQFARVVGLRSISSNAL |
| NO: 486 | p.G639V | | |
| SEQ ID | 6932_GABRB1_ | Mutation/Peptide | TLDNRVADQLWVQDTYFLNDKKSFV |
| NO: 487 | p.P119Q | | |
| SEQ ID | 6932_PCDHAC2_ | Mutation/Peptide | RERQLFSIDASTWEVRVIGGLDYEE |
| NO: 488 | p.G315W | | |
| SEQ ID | 6932_ZFPM2_ | Mutation/Peptide | LDVTWQGVEDNKKNCIVYSKEDIFP |
| NO: 489 | p.N170K | | |
| SEQ ID | 6932_ZFPM2_ | Mutation/Peptide | LDVTWQGVEDNKKNCIVYSKGGQLW |
| NO: 490 | Np.170K* | | |
| SEQ ID | 6932_BCORL1_ | Mutation/Peptide | ANIYPRCSVNGKLTSTQVLPVGWSP |
| NO: 491 | p.P823L | | |
| SEQ ID | 6932_ZNRF3_ | Mutation/Peptide | GEPWPGPASPSGDAAWR |
| NO: 492 | p.D556fs | | |
| SEQ ID | 0025_ANO5_ | Mutation/Peptide | SGATVTLWMSLVITSMVAVIVYRLS |
| NO: 493 | p.V634I | | |
| SEQ ID | 0025_CHST1_ | Mutation/Peptide | RRVMLGASRDLLWSLYDCDLYFLEN |
| NO: 494 | p.R125W | | |
| SEQ ID | 0025_CCDC88B_ | Mutation/Peptide | KQKLVEKIMDQYHVLEPVPLPRTKK |
| NO: 495 | p.R1300H | | |
| SEQ ID | 0025_TENM4_ | Mutation/Peptide | TTDIISVANEDGQRVAAILNHAHYL |
| NO: 496 | p.R2592Q | | |
| SEQ ID | 0025_KRAS_ | Mutation/Peptide | MTEYKLVVVGACGVGKSALTIQLI |
| NO: 497 | p.G12C | | |
| SEQ ID | 0025_SCAF11_ | Mutation/Peptide | RRQSQSRSPKRDSTRESRRSESLSP |
| NO: 498 | p.T945S | | |
| SEQ ID | 0025_AMDHD1_ | Mutation/Peptide | AGGGIHFTVERTCQATEEELFRSLQ |
| NO: 499 | p.R127C | | |
| SEQ ID | 0025_SLC25A29_ | Mutation/Peptide | RGVNRGMVSTLLCETPSFGVYFLTY |
| NO: 500 | p.R94C | | |
| SEQ ID | 0025_ZFYVE19_ | Mutation/Peptide | SLELDYHTSSCFQGTMVKADCPVPI |
| NO: 501 | p.R60Q | | |
| SEQ ID | 0025_FGF7_ | Mutation/Peptide | RGKKTKKEQKTAYFLPMAIT |
| NO: 502 | p.H187Y | | |
| SEQ ID | 0025_MFGE8_ | Mutation/Peptide | SYARLDKQGNFNDWVAGSYGNDQWL |
| NO: 503 | p.A277D | | |
| SEQ ID | 0025_ZNF276_ | Mutation/Peptide | SMVHPLTQTQDKVLPLEAEPPPGPP |
| NO: 504 | p.A372V | | |
| SEQ ID | 0025_FBF1_ | Mutation/Peptide | RRENEELSARYLLQCQEAEQARAEL |
| NO: 505 | p.S674L | | |
| SEQ ID | 0025_RNF157_ | Mutation/Peptide | GTFCVKPLKQKQIVDGVSYLLQEIY |
| NO: 506 | p.V240I | | |
| SEQ ID | 0025_RNF213_ | Mutation/Peptide | APHKKVGFVGISDWALDPAKMNRGI |
| NO: 507 | p.N2935D | | |
| SEQ ID | 0025_DDX39A_ | Mutation/Peptide | PSEVQHECIPQAFLGMDVLCQAKSG |
| NO: 508 | p.I79F | | |
| SEQ ID | 0025_RHPN2_ | Mutation/Peptide | TRQMGLLFTWYDCLTGVPVSQQNLL |
| NO: 509 | p.S201C | | |
| SEQ ID | 0025_NLRP9_ | Mutation/Peptide | LQRRGDCFAFMHQCIQEFCAAMFYL |
| NO: 510 | p.L446Q | | |
| SEQ ID | 0025_MTOR_ | Mutation/Peptide | LLANDPTSLRKNFSIQRYAVIPLST |
| NO: 511 | p.L2220F | | |
| SEQ ID | 0025_LAX1_ | Mutation/Peptide | HATEYAVGIYDNSMVPQMCGNLTPS |
| NO: 512 | p.A158S | | |
| SEQ ID | 0025_SOX13_ | Mutation/Peptide | PARASQDSADPQTPAQGNFRGSWDC |
| NO: 513 | p.A63T | | |
| SEQ ID | 0025_MN1_ | Mutation/Peptide | LFGQSCLAALSTGCQNMIASLGAPN |
| NO: 514 | p.A831G | | |
| SEQ ID | 0025_RPS19BP1_ | Mutation/Peptide | GLELLAASEAPRYPPGQAKPRGAPV |
| NO: 515 | p.D21Y | | |
| SEQ ID | 0025_KIAA0930_ | Mutation/Peptide | NTFQGVIFQGSICYEALKKVYDNRV |
| NO: 516 | p.R208C | | |
| SEQ ID | 0025_CMBL_ | Mutation/Peptide | DKPYIDEARRNLTEWLNKYM |
| NO: 517 | p.I238T | | |
| SEQ ID | 0025_GCNT2_ | Mutation/Peptide | TKYVHQELLNHKKSYVIKTTKLKTP |
| NO: 518 | p.N241K | | |
| SEQ ID | 0025_ECI2_ | Mutation/Peptide | KDPGNEVKLKLYGLYKQATEGPCNM |
| NO: 519 | p.A37G | | |
| SEQ ID | 0025_GRM8_ | Mutation/Peptide | CFSYAALLTKINHIHRIFEQGKKSV |
| NO: 520 | p.R672H | | |
| SEQ ID | 0025_KLRG2_ | Mutation/Peptide | AGAGLEPSSKKKLPSPRPGSPRVPP |
| NO: 521 | p.P70L | | |
| SEQ ID | 0025_RPL8_ | Mutation/Peptide | RFKKRTELFIAAKGIHTGQFVYCGK |
| NO: 522 | p.E80K | | |
| SEQ ID | 0025_GFRA2_ | Mutation/Peptide | LFCSCQDQACAEHRRQTILPSCSYE |
| NO: 523 | p.R246H | | |
| SEQ ID | 0025_PTCH1_ | Mutation/Peptide | RPHRPEWVHDKAYYMPETRLRIPAA |
| NO: 524 | p.D803Y | | |
| SEQ ID | 0025_NYX_ | Mutation/Peptide | LRTLNLGGNALDHVARAWFADLAEL |
| NO: 525 | p.R268H | | |
| SEQ ID | 0025_ARID1A_ | Mutation/Peptide | VKIVQKNDPFVVEISLGVCRSLTVA |
| NO: 526 | p.D1825fs-1 | | |
| SEQ ID | 0025_ARID1A_ | Mutation/Peptide | KNDPFVVEISLGVCRSLTVACCTGG |
| NO: 527 | p.D1825fs-2 | | |
| SEQ ID | 0025_ARID1A_ | Mutation/Peptide | VVEISLGVCRSLTVACCTGGLVGGT |
| NO: 528 | p.D1825fs-3 | | |
| SEQ ID | 0025_ARID1A_ | Mutation/Peptide | LGVCRSLTVACCTGGLVGGTPLSIS |
| NO: 529 | p.D1825fs-4 | | |
| SEQ ID | 0025_ARID1A_ | Mutation/Peptide | SLTVACCTGGLVGGTPLSISRPTSR |
| NO: 530 | p.D1825fs-5 | | |
| SEQ ID | 0025_ARID1A_ | Mutation/Peptide | CCTGGLVGGTPLSISRPTSRARQSC |
| NO: 531 | p.D1825fs-6 | | |
| SEQ ID | 0025_ARID1A_ | Mutation/Peptide | LVGGTPLSISRPTSRARQSCCLPGL |
| NO: 532 | p.D1825fs-7 | | |
| SEQ ID | 0025_ARID1A_ | Mutation/Peptide | PLSISRPTSRARQSCCLPGLTHPAH |
| NO: 533 | p.D1825fs-8 | | |
| SEQ ID | 0025_ARID1A_ | Mutation/Peptide | ISRPTSRARQSCCLPGLTHPAHQPL |
| NO: 534 | p.D1825fs-9 | | |
| SEQ ID | 0025_ARID1A_ | Mutation/Peptide | PTSRARQSCCLPGLTHPAHQPLGSM |
| NO: 535 | p.D1825fs-10 | | |

The present disclosure also provides recombinant vectors expressing a TCR, or an antigen-binding portion thereof, that are disclosed herein. Production of recombinant vectors is well-known in the art, and a variety of vectors may be utilized, including viral or non-viral vectors.

The present disclosure also provides recombinant vectors comprising a polycistronic expression cassette comprising a transcriptional regulatory element operably linked to a polycistronic polynucleotide. The present disclosure provides recombinant polycistronic nucleic acid vectors comprising at least three cistrons, wherein the first cistron encodes an α chain of an artificial T-cell receptor (TCR), the second cistron encodes a β chain of an artificial TCR, and the third cistron encodes a fusion protein that comprises IL-15 and IL-15Rα (e.g., mbIL15), or a functional fragment or functional variant thereof. In some embodiments, the polycistronic nucleic acid further comprises a fourth cistron that encodes a marker protein (e.g., HER1t). In some embodiments, the cistrons are separated by polynucleotide sequence that comprise 2A elements. Any of the TCR alpha or beta chain sequences disclosed herein may be used in the recombinant vectors. Non-limiting examples of the 2A element sequences, the IL-15 sequences, and the sequences are known in the art, e.g., as provided in PCT publication WO 2022/183167, which is incorporated by reference herein in its entirety.

In some embodiments, the recombinant vector comprises a polycistronic expression cassette, where the polycistronic expression cassette comprises a transcriptional regulatory element operably linked to a polycistronic polynucleotide that comprises: a first polynucleotide sequence that encodes a T cell receptor (TCR) alpha chain comprising an alpha chain variable (Vα) region and an alpha chain constant (Cα) region; a second polynucleotide sequence that comprises a first 2A element; a third polynucleotide sequence that encodes a TCR beta chain comprising a beta chain variable (Vβ) region and a beta chain constant (Cβ) region; a fourth polynucleotide sequence that comprises a second 2A element; and a fifth polynucleotide sequence that encodes a fusion protein that comprises IL-15, or a functional fragment or functional variant thereof, and IL-15Rα, or a functional fragment or functional variant thereof. As provided in PCT publication WO 2022/183167, the recombinant vector may comprise the five polynucleotide sequence in any order from 5′ to 3′.

In some embodiments, transgenes of the recombinant vector or any vectors used in the present disclosure are introduced into an immune effector cell via synthetic DNA transposable elements, e.g., a DNA transposon/transposase system, e.g., Sleeping Beauty (SB). SB belongs to the Tc1/mariner superfamily of DNA transposons. DNA transposons translocate from one DNA site to another in a simple, cut-and-paste manner. Transposition is a precise process in which a defined DNA segment is excised from one DNA molecule and moved to another site in the same or different DNA molecule or genome.

Exemplary DNA transposon/transposase systems include, but are not limited to, Sleeping Beauty (see, e.g., U.S. Pat. Nos. 6,489,458, 8,227,432, the contents of each of which are incorporated by reference in their entirety herein), piggyBac transposon system (see e.g., U.S. Pat. No. 9,228,180, Wilson et al, “PiggyBac Transposon-mediated Gene Transfer in Human Cells,” Molecular Therapy, 15:139-145 (2007), the contents of each of which are incorporated by reference in their entirety herein), piggyBac transposon system (see e.g., Mitra et al., “Functional characterization of piggyBac from the bat Myotis lucifugus unveils an active mammalian DNA transposon,” Proc. Natl. Acad. Sci USA 110:234-239 (2013), the contents of which are incorporated by reference in their entirety herein), TcBuster (see e.g., Woodard et al. “Comparative Analysis of the Recently Discovered hAT Transposon TcBuster in Human Cells,” PLOS ONE, 7 (11): e42666 (November 2012), the contents of which are incorporated by reference in their entirety herein), and the Tol2 transposon system (see e.g., Kawakami, “Tol2: a versatile gene transfer vector in vertebrates,” Genome Biol. 2007; 8 (Suppl 1): S7, the contents of each of which are incorporated by reference in their entirety herein). Additional exemplary transposon/transposase systems are provided in U.S. Pat. Nos. 7,148,203; 8,227,432; US20110117072; Mates et al., Nat Genet, 41 (6):753-61 (2009); and Ivies et al., Cell, 91 (4):501-10, (1997), the contents of each of which are incorporated by reference in their entirety herein).

In some embodiments, the transgenes described herein are introduced into an immune effector cell via the SB transposon/transposase system. The SB transposon system comprises a SB a transposase and SB transposon(s). The SB transposon system can comprise a naturally occurring SB transposase or a derivative, variant, and/or fragment that retains activity, and a naturally occurring SB transposon, or a derivative, variant, and/or fragment that retains activity. An exemplary SB system is described in, Hackett et al., “A Transposon and Transposase System for Human Application,” Mol Ther 18:674-83, (2010), the entire contents of which are incorporated by reference herein.

In some embodiments, the recombinant vector comprises a Left inverted terminal repeat (ITR), i.e., an ITR that is 5′ to an expression cassette, and a Right ITR, i.e., an ITR that is 3′ to an expression cassette. The Left ITR and Right ITR flank the polycistronic expression cassette of the vector. In some embodiments, the Left ITR is in reverse orientation relative to the polycistronic expression cassette, and the Right ITR is in the same orientation relative to the polycistronic expression cassette. In some embodiments, the Right ITR is in reverse orientation relative to the polycistronic expression cassette, and the Left ITR is in the same orientation relative to the polycistronic expression cassette.

In some embodiments, the Left ITR and the Right ITR are ITRs of a DNA transposon selected from the group consisting of a Sleeping Beauty transposon, a piggyBac transposon, TcBuster transposon, and a Tol2 transposon. In some embodiments, the Left ITR and the Right ITR are ITRs of the Sleeping Beauty DNA transposon.

The present disclosure further provides a population of cells that comprise the recombinant vectors disclosed herein. In one aspect, the recombinant vector or the polynucleotide is integrated into the genome of the population of cells. In one aspect, the cells are immune effector cells. In certain aspects, the immune effector cells are selected from the group consisting of T cells, natural killer (NK) cells, B cells, mast cells, and myeloid-derived phagocytes.

The present disclosure also provides a population of cells comprising a polycistronic expression cassette comprising: a. a first cistron comprising a polynucleotide sequence that encodes a fusion protein that comprises IL-15, or a functional fragment or functional variant thereof, and IL-15Rα, or a functional fragment or functional variant thereof; b. a second cistron comprising a polynucleotide sequence that encodes a TCR beta chain comprising a VB region and a Cβ region; and c. a third cistron comprising a polynucleotide sequence that encodes a TCR alpha chain comprising a Vα region and a Cα region.

In some embodiments, the recombinant vectors disclosed herein comprise a polynucleotide sequence that encodes an amino acid sequence at least 90%, 91%, 92%, 93%, 94%, 95%, 96%, 97%, 98%, 99%, or 100% identical to any of the TCR alpha or beta chain sequences provided in Tables 1-79 herein.

The present disclosure provides a pharmaceutical composition comprising a population of cells as disclosed herein. In one aspect, the pharmaceutical composition comprises a pharmaceutically acceptable carrier.

It is contemplated that the TCRs identified by the methods disclosed herein, the antigen-binding portions thereof, populations of cells, and pharmaceutical compositions can be used in methods of treating or preventing medical conditions, such as cancer. Without being bound to a particular theory or mechanism, the TCRs, or the antigen-binding portions thereof, are believed to bind specifically to a mutated amino acid sequence encoded by a cancer-specific mutation, such that the TCR, or the antigen-binding portion thereof, when expressed by a cell, is able to mediate an immune response against a target cell expressing the mutated amino acid sequence. In this regard, an aspect of the disclosure provides a method of treating or preventing cancer in a mammal, comprising administering to the mammal any of the pharmaceutical compositions, isolated pairs of TCR α and β chain sequences, antigen-binding portions thereof, or populations of cells described herein, in an amount effective to treat or prevent cancer in the mammal.

Aspects of the disclosure include a cell or cells encompassed by the disclosure for use in the treatment of a medical condition, such as cancer or a premalignant condition, in a subject. The cells may be used for any type of cancer, including neuroblastoma, breast cancer, cervical cancer, ovary cancer, endometrial cancer, melanoma, bladder cancer, lung cancer, pancreatic cancer, colon cancer, prostate cancer, hematopoietic tumors of lymphoid lineage, leukemia, acute lymphocytic leukemia, chronic lymphocytic leukemia, B-cell lymphoma, Burkitt's lymphoma, multiple myeloma, Hodgkin's lymphoma, Non-Hodgkin's lymphoma, myeloid leukemia, acute myelogenous leukemia (AML), chronic myelogenous leukemia, thyroid cancer, thyroid follicular cancer, tumors of mesenchymal origin, fibrosarcoma, rhabdomyosarcomas, melanoma, uveal melanoma, teratocarcinoma, neuroblastoma, glioma, glioblastoma, benign tumor of the skin, renal cancer, anaplastic large-cell lymphoma, esophageal squamous cells carcinoma, hepatocellular carcinoma, follicular dendritic cell carcinoma, intestinal cancer, muscle-invasive cancer, seminal vesicle tumor, epidermal carcinoma, spleen cancer, bladder cancer, head and neck cancer, stomach cancer, liver cancer, bone cancer, brain cancer, cancer of the retina, biliary cancer, small bowel cancer, salivary gland cancer, cancer of uterus, cancer of testicles, cancer of connective tissue, prostatic hypertrophy, myelodysplasia, Waldenstrom's macroglobinaemia, nasopharyngeal, neuroendocrine cancer myelodysplastic syndrome, mesothelioma, angiosarcoma, Kaposi's sarcoma, carcinoid, oesophagogastric, fallopian tube cancer, peritoneal cancer, papillary serous mullerian cancer, malignant ascites, gastrointestinal stromal tumor (GIST), or a hereditary cancer syndrome selected from Li-Fraumeni syndrome and Von Hippel-Lindau syndrome (VHL).

The examples of the present disclosure are offered by way of illustration and explanation, and are not intended to limit the scope of the present disclosure.

### EXAMPLES

### Example 1: Workflow to Identify Tumor Specific TCRs from Patient Derived TIL and Dissociated Primary Tumors

### 1.1 TCR Identification and Screening Platform

The fundamental basis for this unbiased TCR identification and screening platform is illustrated in FIG. 1. Initially, single-cell gene expression data (e.g., 5′ GEX Analysis) from T cells is utilized to perform unsupervised clustering analysis by employing dimensionality reduction methods such as principal component analysis (PCA), t-distributed Stochastic Neighbor Embedding (tSNE), or Uniform Manifold Approximation and Projection (UMAP) (FIG. 1, STEP 1). Merging the clustered single-cell gene expression analysis with paired, full-length TCR sequences then enables the identification of TCR clonotypes present in each of the distinct clusters. TCR sequences are then selected from the overall single-cell dataset based on frequency, cluster attributes, specific-gene expression signatures, or other criteria employed to increase the likelihood of obtaining TCRs with desired reactivity (i.e., antigen/HLA specificity) (FIG. 1, STEP 2). Selected paired, full-length TCR sequences are then reconstructed in silico, from which, expression plasmids encoding the TCR α and β chains synthesized (FIG. 1, STEP 3). These TCR expression cassettes are then cloned into transposon or other non-viral gene transfer vector to enable quick translation into process development, manufacturing, and clinical applications. TCR-expression plasmids are then transiently expressed in a cell line (e.g., Jurkat or SUP-T1) or primary cell (e.g., human ex vivo expanded T cells) that will signal upon TCR recognition of cognate antigen:HLA complexes on the surface of antigen presenting cells (APCs) (FIG. 1, STEP 4). Antigen presenting cells (APCs) are classical professional APCs such as dendritic cells (DCs) or an artificial antigen presenting cell (e.g., COS-7 or 293-HEK). APCs either endogenously express the requisite HLA allele(s) or are transfected with HLA expression plasmids. Antigens are introduced to the APCs either by genetic transfer to antigen encoding plasmids (e.g., Tandem Minigene (TMG) plasmids) or by the pulsing of peptide pools. One aspect of the APC system used is that multiple HLA alleles and antigens are screened within the same set of APCs, thus enable high-throughput assessment of hundreds to thousands of antigen:HLA combinations. Co-culture of the TCR modified cells and APCs is then performed to identify reactive TCRs (FIG. 1, STEP 5). Reactive TCRs are those that are found to recognize one of the antigen:HLA conditions tested. These reactive TCRs are then further evaluated in vitro to confirm the findings and deconvolute the multiplexed HLA/antigen. Once all reactive TCRs are identified from a specimen, that binary outcome (reactive vs non-reactive) for each TCR can be mapped back to the initial gene-expression cluster analysis (FIG. 1, STEP 6). By mapping the reactive TCRs back to the gene-expression data, gene signatures or biomarkers which are enriched in the reactive TCR cell population are elucidated and used to further improve and refine the initial selection of TCRs for screening. Overall, this fundamental process is used to identify TCR sequences and their associated antigen and HLA specificity with a high level of confidence and accuracy from complex starting materials such as tumor tissues or blood samples.

1.2 Screening of TCRs from TILs

In practice, the steps of the above-described workflow (FIG. 1) can be further broken down into critical processes as shown in FIG. 2 for screening of TCRs obtained from TILs. The process illustrated in FIG. 2 correspond to FIG. 1 STEPs 1-5. The workflow illustrated in FIG. 2 is further broken into two parallel processes (indicated with either Alpha [i.e., A, B, C, etc.] or Numeric [i.e., 1, 2, 3, etc.] STEP designators) that diverge from a common starting point (STEP 1/A) and converge at a common finishing point (STEP 8/F). STEP 1/A to STEP 6 illustrate the workflow from TILs isolation to generation of cells expressing TILs-derived TCRs. STEP 1/A to STEP D illustrate the workflow from patient mutation and HLA calling to the generation of APCs expressing the patient matched HLA and mutation-derived antigens (e.g., neoantigens).

STEP 1-6 (TCR): Initially, in STEP 1/A, a tumor sample is obtained from a cancer patient (FIG. 2). This tumor sample is dissociated into a single-cell suspension and TILs are isolated by fluorescent activated cell sorting (FACS) by staining dissociated tumor samples for lymphocyte, T cell, and live cell markers (FIG. 2, STEP 2). Single-cell transcriptomics is then performed on the sorted TILs to obtain gene expression and TCR V(D)J sequences (FIG. 2, STEP 3). Bioinformatic analysis of the gene-expression data is used to cluster cells based on transcriptional similarities to aid in the selection of TCR sequences for in vitro evaluation (FIG. 2, STEP 4). Once selected, TCRs are reconstructed in silico and synthesized in expression vectors (FIG. 2, STEP 5) to enable transgenic expression of the TCRs in cells capable of forming a functional TCR complex with CD3 subunits and CD4/CD8 co-receptors. These cells are engineered to express any or all necessary protein components of the TCR signaling complex or downstream signaling components. Moreover, these components are modified to further enhance their function in the platform (e.g., CD4 with amino acid substitutions at Q40Y, T45W, P48L, S60R, and/or D63R to enhance affinity to MHC-Class II). Wang et al. 2011 PNAS, 108 (38):15960-15965. TCR expression vectors are transferred into the Reporter cells to generate Reporter TCR-T cells (FIG. 2 STEP 6).

STEP A-D (Antigen/HLA): In parallel to STEPs 1-6, nucleic acids (DNA and RNA) can be extracted from the tumor sample (FIG. 2, STEP 1/A). Using Whole Exome Sequencing (WES) and RNA Sequencing (RNAseq) to generate genomic and transcriptional datasets, a bioinformatics pipeline is employed to determine somatic mutations present in the tumor as well as the patient's germline HLA typing (FIG. 2, STEP B). Somatic mutations are ranked and concatenated so that TMGs and peptide pools can be synthesized (FIG. 2, STEP C). These reagents provide the antigen component of the screening assay. Similarly, sequences of the called HLA alleles are synthesized in expression vectors to provide the HLAs necessary for the screening assay. Antigen presenting cells, such as COS-7, are then modified either by stable or transient transfection to express the requisite Class I or Class II HLA alleles either in single-plex or multiplexed within the same cells (FIG. 2, STEP D). Antigen is provided to the APCs either by transfection of relevant TMGs (either as plasmid DNA or in vitro transcribed RNA) and/or peptide pools containing antigens derived from the tumor's somatic mutations identified. With both the HLA and antigen provided to the APCs, they are able to present peptide:HLA complexes to T cells in vitro.

STEP 7/E-8/F: Reporter cells expressing transgenic TCRs (FIG. 2, STEP 6) and antigen/HLA-modified APCs (FIG. 2, STEP D) are co-cultured together at a pre-determined ratio of Reporter cells (E) to APCs (T), typically approximately 4:1 to 8:1 (FIG. 2, STEP 7/E). Positive control wells containing PMA/Ionomycin or coated with H57-597 antibody (anti-transgenic TCR) with the TCR-modified Reporter cells are also set up. Negative control wells of Reporter cells alone or co-cultured with APCs modified with HLA-only, irrelevant antigens, or non-transfected are also set up. All conditions are typically evaluated in duplicate. After the co-culture period, reporter activity (i.e., luciferase activity) is quantified in each co-culture and control well (FIG. 2, STEP 8/F). For a given TCR, the reporter activity is compared across all antigen:HLA conditions evaluated to determine if there is a condition with increased reporter activity which indicates that the transgenic TCR recognized an antigen:HLA combination present in that well. Because initial screening multiplexes multiple HLA alleles and antigens, when there is specific TCR activity observed, STEP 7/E and 8/F are repeated using APCs modified with single HLA and antigens to elucidate the exact specificity of the TCR. Moreover, minimal epitopes can be determined using this co-culture method. Overall, this workflow enables the identification of TCR sequences and the empirical determination of specificity to selected antigens and HLA alleles.

### 1.3 Relationship Between TCR-Based and TILs-Based Screening Methods

FIG. 3 illustrates the relationship between a TCR-based screening method (below dotted line) and TILs-based screening method (above dotted line). The TCR-based screening method is as described above in the description of FIG. 2 wherein TCR sequences, somatic mutations, and HLA-typing is obtained from primary tumor samples and utilized to screen selected TCRs for reactivity to tumor neoantigens using a co-culture reporter system. Similarly, TILs screening starts with a primary tumor sample obtained from a cancer patient. TILs are expanded from the tumor using standard TILs expansion methods (high-concentration IL-2, feeder cells, muromonab-CD3 (OKT3)). Expanded TILs are then co-cultured in an IFN-γ ELISpot with APCs modified to express the relevant HLA alleles and antigens identified from WES and RNAseq data from the tumor. This is performed in a similar plate layout to TCR screening where multiple HLA alleles and antigens are multiplexed in the same wells, thus increasing the throughput of the assay. Positive controls include PMA/Ionomycin. Negative controls include TILs alone, APCs alone, TILs+APCs without HLA and/or antigen, and no cells. After the overnight co-culture, cells are harvested from the IFN-γ ELISpot and the plate is developed to measure the number of spot-forming colonies (SFCs) of each well. The harvested TILs are also stained and evaluated for upregulation of 4-1BB or other activation molecules (e.g., OX40). TILs from co-culture conditions which produce increased numbers of SFCs and/or activation marker expression are then sorted for either total live T cells or for T cells expressing the activation marker. Single cell gene expression and TCR V(D)J sequencing is then performed on the sorted cells. T cells from a negative control co-culture (typically APCs modified with HLA alone or with HLA and irrelevant antigen) are similarly sorted and analyzed by single-cell transcriptomics. Using the single-cell gene expression data, clusters of activated TILs can be identified. Paired, full-length TCR sequences from these activation clusters are then reconstructed into TCR expression plasmids and screened using the TCR screening methods described in FIG. 2. Overall, FIG. 3 illustrates parallel workflows with either ex vivo expanded TILs or sorted TILs are utilized to identify tumor-reactive TCRs with potential therapeutic applications in oncology. These general methods are applied to identify therapeutically useful TCRs in other disease indications (e.g., inflammation, auto-immune, etc.) with the appropriate starting material (e.g., a biopsy of inflamed colon from Crohn's disease patient or a plaque of a patient with psoriasis).

### Example 2: Development of TCR Screening Methods

### 2.1 General Methods Used in the Examples

### 2.1.1 Nucleic Acid Isolation and Assessment

Tumor samples are obtained as either dissociated tumors or frozen tissue. To isolate DNA and RNA from dissociated cells, cells are processed using Qiagen AllPrep DNA/RNA Mini kit per the manufacturer's protocol. To isolate DNA and RNA from tissue, frozen tissue is disrupted using a mortar and pestle and homogenized using QIAshredder homogenizers. The homogenized tissue is processed through the Qiagen AllPrep DNA/RNA Mini kit according to the manufacturer's protocol.

Matched normal samples are obtained either as whole blood or as PBMCs. The Qiagen DNeasy Blood & Tissue kit is used to isolate DNA from 200 μL of whole blood per the manufacturer's protocol and including the optional RNaseA. To isolate DNA and RNA from PBMCs, cells are processed using Qiagen AllPrep DNA/RNA Mini kit per the manufacturer's protocol.

Isolated nucleic acids are quantified by fluorescence spectrometry using the Life Technologies Qubit dsDNA BR Assay kit. Nucleic acids are assessed for fragment size by automated electrophoresis using the Agilent TapeStation 4150. Genomic DNA is assessed using the Agilent Genomic DNA ScreenTape System and RNA is assessed using the Agilent RNA ScreenTape System.

### 2.1.2 RNAseq

To assess gene expression, RNA from tumors are processed through Illumina RNA Prep with Enrichment with an input of 100 ng. Pre-capture libraries are enriched via hybridization with the Illumina Exome Panel.

Molarity of final libraries is determined using the size for fragments between 100 and 1000 bp on the Agilent TapeStation 4150 (Agilent High Sensitivity D1000 ScreenTape assay) and library concentration from Qubit 4 (Life Technologies Qubit dsDNA BR Assay kit). Libraries are pooled with a 1% PhiX spike-in. The library pool is clustered and sequenced at 2×76 on an Illumina NextSeqDx 550 using a 150 cycle High Output kit for a target coverage of 150 M reads. Libraries are subject to on-board demultiplexing to yield FASTQ files.

The raw RNA-seq reads are aligned to the hg19 genome using Spliced Transcripts Alignment to a Reference (STAR) with the two-step procedure. Then Cufflinks is applied to the obtained BAM file to calculate the Fragments Per Kilobase of transcript per Million mapped reads (FPKM) value of each gene. The FPKM values are converted to deciles to represent ten gene expression levels.

### 2.1.3 Single Cell RNAseq

To sequence TCRs, dissociated tumor cells are processed through the Chromium Next GEM Single Cell 5′ Reagent Kit v2 from 10× Genomics targeting 10,000 cells when possible. The resulting cDNA is processed through the Chromium Single Cell Human TCR Amplification Kit VDJ per manufacturer's recommendations.

Molarity of final libraries is determined using the size for fragments between 100 and 1000 bp on the Agilent TapeStation 4150 (Agilent High Sensitivity D1000 ScreenTape assay) and library concentration from Qubit 4 (Life Technologies Qubit dsDNA BR Assay kit). Libraries are pooled with a 1% PhiX spike-in. The library pool is clustered and sequenced at 26+96 on an Illumina NextSeqDx 550 using a 150 cycle High Output kit for a target coverage of 5000 reads per cell for VDJ and 20,000 reads per gene expression library. Raw bcl files are yielded.

### 2.1.4 Analysis of the 10× Gene Expression (GEX) and VDJ Sequencing Data

The GEX and VDJ sequencing data are preprocessed using the CellRanger toolkit (version 5.1) provided by 10× Genomics. The BCL files from the Illumina sequencer are converted to raw FASTQ files. The FASTQ files for the GEX and VDJ experiments are processed separately. GEX reads are aligned to the human GRCh38 reference genome. Cell barcodes assignment and UMI counting are then performed to create a single-cell gene expression matrix. Doublets and cells with >10% mitochondria gene counts are filtered out in the study. Then the raw read counts are normalized and scaled using Seurat. About 2,000 highly variable genes are identified using the FindVariableGenes module. Next, principal component analysis (PCA) and uniform manifold approximation and projection (UMAP) are performed for dimension reduction and a shared nearest neighbor (SNN) algorithm is applied to cluster the cells.

The raw VDJ reads are assembled into contigs using a graph-based algorithm with the aid of the pre-built reference sequence from the IMGT database. Cells with identical productive V(D)J transcripts are placed into a same clonotype.

### 2.1.5 Integrating the 10×GEX and VDJ Data

For each TCR clonotype, the corresponding cells in V(D)J are projected to the identified clusters in the GEX data. The full-length FASTA sequences of both the TRA and TRB chains, as well as the amino acid sequences of the CDR3 regions for each clonotype are also reported.

### 2.1.6 Whole Exome Sequencing

Whole exome sequencing experiments (WES) are performed for the peripheral blood and the tumor tissue of each patient. Somatic single nucleotide variants (SNVs), short insertions and deletions (indels), copy number alterations (CNAs), class I and II HLA types are detected by comparing the tumor versus the normal sequencing data. Each mutant peptide is predicted in silico if it can give rise to a neoantigen. Bulk RNA-Seq is also performed on the tumor tissue to quantify the expression level of each gene.

Between 100 and 200 ng of genomic DNA is fragmented enzymatically for 100 bp reads using the Agilent SureSelect Enzymatic Fragmentation Kit. Fragmented DNA is processed through the SureSelect XT HS2 DNA System using v7 probes.

Pre-capture libraries the size for fragments between 100 and 1000 bp on the Agilent TapeStation 4150 (Agilent High Sensitivity D1000 ScreenTape assay) and library concentration from Qubit 4 (BR). A total of 1000 ng of pre-capture library is input into hybridization.

Molarity of final libraries is determined using the fragment size between 100 and 1000 bp on the Agilent TapeStation 4150 (Agilent High Sensitivity D1000 ScreenTape assay) and concentration from Qubit 4 (Life Technologies Qubit dsDNA HS Assay Kit). Libraries are pooled with a 1% PhiX spike-in. The library pool is clustered and sequenced at 2×101 on an Illumina NextSeqDx 550 using a 300 cycle High Output kit for a target coverage of 200× and 100× for tumor and normal libraries, respectively. Libraries are subject to on-board demultiplexing to yield FASTQ files.

### 2.1.7 Jurkat NFAT Cell Generation

Jurkat NFAT cells are infected Lentivirus (pGenLenti-CD8A_P2A_CD8B_IRES_Puro) and then selected with puromycin (0.2 μg/mL). Peripheral Blood Mononuclear Cells (PBMCs) from 3 different donors are irradiated and seeded in a 96 multiwell U bottom plate at 100 k/well. Puromycin selected stable pools of peptides are seeded at 0.5 cell/well on top of irradiated PBMCs (96 multiwell plates) to generate single clones. Single clones are cultured for 1 week with IL-2 (50 IU/mL) and Phytohaemagglutinin-L (PHA-L) (0.25 μg/mL). Second week cell medium is replaced with 100 IU/mL of IL-2. Grown back clones are evaluated for higher CD3/CD8 expression and higher luciferase signal/noise ratio (PMA/Ionomycin vs untreated). Clone #41 (having >95% CD8 expression and >150 signal to noise ratio) is selected. In order to better screen class II TCRs, #41 clone is infected with CD4 lentivirus (pGenLenti-CD4_IRES_Puro) to boost CD4 expression. After lentivirus infection CD4 expression is increased to more than 95%.

### 2.1.8 Mutation Calling, HLA-Typing and Neoantigen Prediction

The raw WES reads are aligned to the human hg19 reference genome using Burrows-Wheeler Aligner (BWA) (version 0.7.5a). Duplicate reads are marked using Picard's “MarkDuplicates” module. The “IndelRealigner” and “BaseRecalibrator” modules of the Genome Analysis Toolkit are then applied to the obtained BAM files for indel realignment and base quality recalibration. In our workflow, five mutation detection algorithms are applied to the obtained BAM files: Mutect, MuSE, Varscan2, Mutect2 and Strelka, where all of them are used to detect single nucleotide variants (SNVs) and the last three are used to detect short insertions or deletions (indels). Only Mutect2 is used to detect multi-nucleotide variants (MNVs). An SNV is reported if it can be detected by at least three out of the five algorithms. An indel is reported if it can be detected by any of the indel callers.

The detected SNVs are annotated with ANNOVA and VEP and compared with public databases such as dbSNP (Sherry et al., 2001), 1,000 genome (http://www.1000genomes.org/) and ESP6500 (http://evs.gs.washington.edu/EVS/). To ensure accuracy, the following criteria is used to filter the SNV and indel list: allele frequency (AF)>0.05; the coverage is at least 20 reads for the tumor and 10 for the normal; the AF from the normal sample <0.02. Only non-synonymous SNVs, in-frame and frameshift indels are kept for further analysis, as these mutations change the amino acid sequences of the genome and are likely to give rise to neoantigens. For each mutated amino acid that results from a somatic SNV or indel, up to 12 bases are extended to the left and to the right and a peptide sequence of length at most 25 bases (25-mer) is obtained. Since a neoantigen's length ranges from 8-25 bases, it ensures that any potential neoantigen resulting from the mutation is a subsequence of the 25-mer.

The Sequenza algorithm is used to detect the somatic copy number alterations (CNAs) and tumor purity. Optitype and HLA-VBSeq are applied to infer the class I and II HLAs respectively.

The 25-mer peptide sequences and the HLA types of each patient are input together to netMHCpan4.1 to predict if the mutant amino acids can lead to a neoantigen.

### 2.1.9 TCR Plasmid Assembly

Approximately 50 T Cell Receptors (TCRs) are selected per patient by a still-developing method according to their abundance in the assessed sample and the association of their corresponding cells with clusters according to gene expression. TCRs are selected considering whether (1) a cluster expresses CD8 or CD4, (2) the function of genes differentially expressed by that cluster, and (3) the abundance of each TCR. Each analysis yields more than 1000 TCR clonotypes, and these are reduced to approximately 50 clonotypes to move on to synthesis. Each cluster is defined by differentially expressed genes. Each cluster is made up of cells, and each cell is associated with a TCR clonotype. The highest abundance clonotypes in every cluster are included such that a total of approximately 50 clonotypes are synthesized across all clusters, giving preference to clonotypes from clusters that are associated with immune response genes. Similarly, if a patient sample has a Class I or Class II HLA allele that is common in the population, preference is given to clusters that more highly express either CD8 or CD4, respectively.

### 2.1.10 Create Beta and a Gene Sequences in Silico

The raw beta sequence is curated such that any sequence 5′ of the start of the Variable (V) region is replaced with a NheI site, and the entire constant region is replaced with a BspI site. For the α chain, the sequence 5′ of the start of the V region is replaced with an XmaI site, and the constant region is replaced with a SacII site.

Rare codons (defined as codons used <10% according to the Homo sapiens codon usage table) are replaced with more frequently used codons for the same amino acid throughout the beta and a open reading frames. NheI, BspI, XmaI, and SacII restriction sites are eliminated from the open reading frame by replacing codons with other codons encoding the same residues.

### 2.1.11 Plasmid Assembly

Each α and β gene are synthesized and subcloned into pZT2 plasmids using the synthesized restriction sites (NheI and BspEI for beta and XmaI and SacII for α) by GenScript. The final plasmid is prepared in 10 mMTris-HCl, pH 8.0, 1 mM EDTA (TE) with 95%+5% supercoiled plasmid and ≤0.005 EU/μg endotoxin content.

### 2.1.12 Tandem Minigene Plasmid Assembly

When more than 150 non-synonymous mutations are reported for a tumor, the mutations are sorted by gene expression and only the top 150 expressed non-synonymous mutations are included.

### 2.1.13 TMG Assembly in Silico

Amino acid sequences are reverse translated in silico and codon optimized for expression in human cells. BamHI, EcoRI, NotI and NheI restriction sites are removed by replacing codons with others encoding the same residues. A set of up to 15 sequences are concatenated together into one open reading frame called a tandem minigene (TMG). The nucleotide sequence GAG AAT TCG (codes for Glu (E)/Asn (N)/Ser(S)) and has EcoRI site=GAATTC) is added to the 5′ end of the TMG gene, and the nucleotide sequence AAG GAT CCC (codes for K/D/P and has BamHI site=GGATCC) is added to the 3′ end of the TMG gene.

### 2.1.14 TMG Plasmid Synthesis

The TMG, together with the added restriction sites, is synthesized and cloned (GenScript) into masterTMG_pcDNA3.1 (+) mammalian expression vector with EcoRI (5′) and BamHI (3′) in frame with existing start and stop codons. The final plasmid is prepared in TE with 95%+5% supercoiled plasmid and ≤0.005 EU/μg endotoxin content.

### 2.1.15 Peptide Design and Synthesis

The same amino acid sequences are synthesized up to 25 aa in length with crude quality (GenScript). For peptide sequences longer than 25 residues, multiple peptides of 25 aa in length are synthesized with start sites at 5 aa intervals. For the last window, the last 25 residues are synthesized in place of a peptide shorter than 25 aa.

### 2.1.16 Human Leukocyte Antigen (HLA) Plasmid Assembly

Peptide sequences for each identified allele are downloaded from the IPD-IMGT/HLA Database (ebi.ac.uk). Each peptide sequence is reverse translated in silico and codon optimized for expression in human cells. The sequence is then synthesized with BamHI and Kozak sites at the 5′ end and an EcoRI and stop codon on the 3′ end (GenScript). The synthesized sequence is cloned into pcDNA3.1 (+) using BamHI and EcoRI. Final plasmids are prepared in TE with 95%+5% supercoiled plasmid and ≤0.005 EU/μg endotoxin content.

### 2.1.17 Neoantigen Specific TCR Screening Process.

On Day 1, COS-7 cells are seeded at 20,000 cells per well (96 multiwell plate) overnight in 37° C. incubator. On Day 2, cell medium is replaced with antibiotic-free DMEM medium before transfection. 150 ng of tandem minigene (TMG) and 300 ng HLA plasmids are transfected using lipofectamine 2000. Three to four HLA plasmids (75-100 ng each) are transfected together in one well to enhance screen efficacy. Each condition includes one or two HLA types including A, B, C, DP, DQ and DR. Twenty-five μL of OptiMEM medium is used to dilute either DNA plasmid (450 ng total) or Lipofectamine (0.6 μL) for each well. DNA tube (A) or lipofectamine tube (B) are mixed well separately and incubated for 5 minutes at room temperature (RT). Tube B is then added to tube A, and the mixture is incubated for 20 minutes at RT. Transfection mix (50 μL) is added to each well and cells are cultured overnight in a 37° C. incubator.

Jurkat NFAT reporter cells are counted and seeded at 1 million/mL with fresh RPMI1640 complete medium overnight to enhance electroporation efficacy (10% Fetal Bovine Serum (FBS) and 1% Pen/Strep). On Day 3, Neon™ transfection system is set up in the Biosafety Cabinet (BSC) with program 1,325v, 10 mins, 3 Pulse. 5 mLs of RPMI without Pen/Strep is added into T25 flask and labeled with corresponding murine-TCR (mTCR) number. Flasks are pre-warmed in 37° C. incubator while preparing electroporation (EP) Jurkat NFAT cells are spun down at 100 g for 10 minutes. Cells are washed with PBS and cell numbers are measured with NC3000. 6 million Jurkat NFAT cells are loaded into 15 mL conical tubes and spun down at 100 g for 10 minutes. During centrifugation, Buffer R (110 μL each) are prepared in Eppendorf tubes and Electrolytic Buffer E2 (3 mL each) are aliquoted in Neon transfection system tubes. Eleven microliters of mTCR plasmids (2 mg/mL) are added to corresponding Eppendorf tubes containing Buffer R and mixed well. The mixture of DNA and Buffer R is loaded to the Neon tubes using specialty Neon pipette tips. When EP is successful, “COMPLETE” shows on the screen in a few seconds after “START” is clicked. Buffer R/DNA mixture is transferred immediately into a T25 flask containing antibiotic-free RPMI medium. H57-597 antibody is utilized to coat plate (1 μg/mL, 25 μL/well) overnight to measure EP efficacy next day. For parsing experiment, peptide is prepared at 50 mg/mL and pulsed at 10 μg/mL to identify neoantigen specificity.

### 2.1.18 Co-Culture

On Day 4, Jurkat NFAT-mTCR cells are counted and co-cultured (100 k/well) on top of transfected COS-7 cells for 4-5 hours. As control, Jurkat NFAT-mTCR cells were also plated on H57 coated plate to perform mTCR functional test. After 4-5 hours incubation, cells from 96 multiwells were transferred to U bottom plates and spined down at 400 g for 5 minutes. Cells were then lysed with 1× passive lysis buffer (100 μL/well) for 15 minutes on an orbital shaker at RT. 50 μL cell lysis were loaded onto OPTIPLATE as well as 100 μL of Promega Luciferase substrate. Luciferase activity was measured immediately with BioTek reader. Jurkat NFAT cells mTCR expression was measured with flow cytometry using antibody cocktail CD3, CD4, CD8A, CD8B and H57. HLA expression of COS-7 cells was measured with flow cytometry using antibody cocktail HLA-A2, HLA-DP, HLA-DQ and HLA-DR.

### 2.1.19 Neoantigen Specific Tumor Infiltrating Leukocytes (TILs) Identification Process.

On Day 1, COS-7 cells are seeded at 20,000 cells per well (96 multiwells) overnight in 37° C. incubator. TILs are thawed and recovered with IL-2 at 3000 IU/μL. On Day 2, cell medium is replaced with antibiotic-free DMEM medium before transfection. A Transfection Mix containing 150 ng of tandem minigene (TMG) and 300 ng HLA plasmids are prepared and transfected into the COS-7 cells using lipofectamine 2000. Two HLA plasmids (150 ng each) are transfected together in one well to enhance screening sensitivity. Each condition only includes one HLA type (A, B, C, DP, DQ and DR). 25 μL of OptiMEM medium is used to dilute either DNA plasmids (450 ng total) or Lipofectamine (0.6 μL) for each well. DNA tube (A) and lipofectamine tube (B) are mixed well and incubated separately for 5 minutes at room temperature (RT). Tube B is added to tube A, and the mixture is incubated for 20 minutes at RT. Transfection mix (50 μL) is added to each well and cells area cultured overnight in a 37° C. incubator. On Day 3, 96 multiwell plates containing COS-7 cells are replaced with fresh medium containing peptide pools. Peptide pools are created by combining the peptides from a given TMG into a pool of equivalent mass ratios of each peptide. Peptides are prepared at 50 mg/mL and pulsed at a final concentration of 10 μg/mL (in well which contains media and COS-7 cells). Peptide pools consist of the synthesized peptides that correspond to the minigenes within a given TMG (i.e., if TMG-1 contains minigenes encoding Peptide 1, Peptide 2, and Peptide 3, a peptide pool containing Peptides 1-3 would be prepared). ELISpot plates are incubated with 70% EtOH (0.22 μm filter, 50 μL/well) for less than 2 mins in the Biosafety Cabinet (BSC) at RT. ELISpot plates are washed 5 times with 200 μL/well with sterile PBS. Anti-interferon gamma capture antibody (1-D1K) is mixed with PBS (100 μL/10 mL/plate) and added 100 μL/well. COS-7 cells are incubated overnight at 4° C. On Day 4, ELISpot plates are washed 5 times with PBS (200 μL/well). Plates are blocked with complete RPMI media (10% FBS), 100 μL/well at room temperature for 1 hour. During the one hour, COS-7 cells are harvested from 96 multiwells using trypsin. TILs are counted and resuspended at 400k/mL. Medium is poured out from the ELISPOT plate. 50 μL of medium, 100 μL of COS-7 cells, and 100 μL of TILs (40,000 cells) are added sequentially to the ELISpot plates. Plates are transferred to 37° C. incubators with 5% CO₂, and incubated for 18-24 hours. On Day 5, the following ELISpot reagents are prepared: 1) IFN-γ biotinylated 7-B6-1 antibody diluted in PBS+0.5% FBS, then filtered with 0.22 μm filter, and 2) wash buffer (PBS+0.05% Tween-20). Cells of each well are mixed via pipetting, then 200 μL of cells are carefully transferred from ELISpot plate to a new 96 U-bottom plate. The cells are later stained for phenotyping using cocktail CD3, CD4, CD8 and 41BB with flow cytometry. ELISpot plates are washed 3 times using buffer made by combining PBS with 0.05% tween 20 in the big basin. Anti-IFN-γ antibody (Biotinylated 7-B6-1 biotin) is diluted with PBS and 0.5% FBS then filtered with 0.22 μm filter (10 μL/10 mL/plate, 100 μL/well). Plates are left at room temperature for 2 hours in the dark covered with aluminum foil. Plates are washed 5 times using PBS with 0.05% tween 20. Streptavidin-ALP is diluted in PBS with 0.05% FBS (10 μL/10 mL) and added at 100 μL/well at room temperature for 1 hr in the dark covered with aluminum foil. Plates are washed 5 times with PBS. 5-Bromo-4-chloro-3-indonyl phosphate, X-phosphate, XP, Nitro-blue-tetrazolium chloride, (BCIP/NBT) Alkaline Phosphatase substrate solution is filtered (0.45 μm) and added at 100 μL to every well. Plates are incubated at room temperature for 10-20 mins until distinct spots can be seen. Tap water is used to wash the plates gently but extensively, then the plates are left out until completely dry. Plates are analyzed using the ELISpot reader. HLA expression of COS-7 cells are measured with flow cytometry using antibodies cocktail HLA-A2, HLA-DP, HLA-DQ and HLA-DR.

### 2.2 Modification of Jurkat Reporter Cells

### 2.2.1 Adding CD8 and CD4

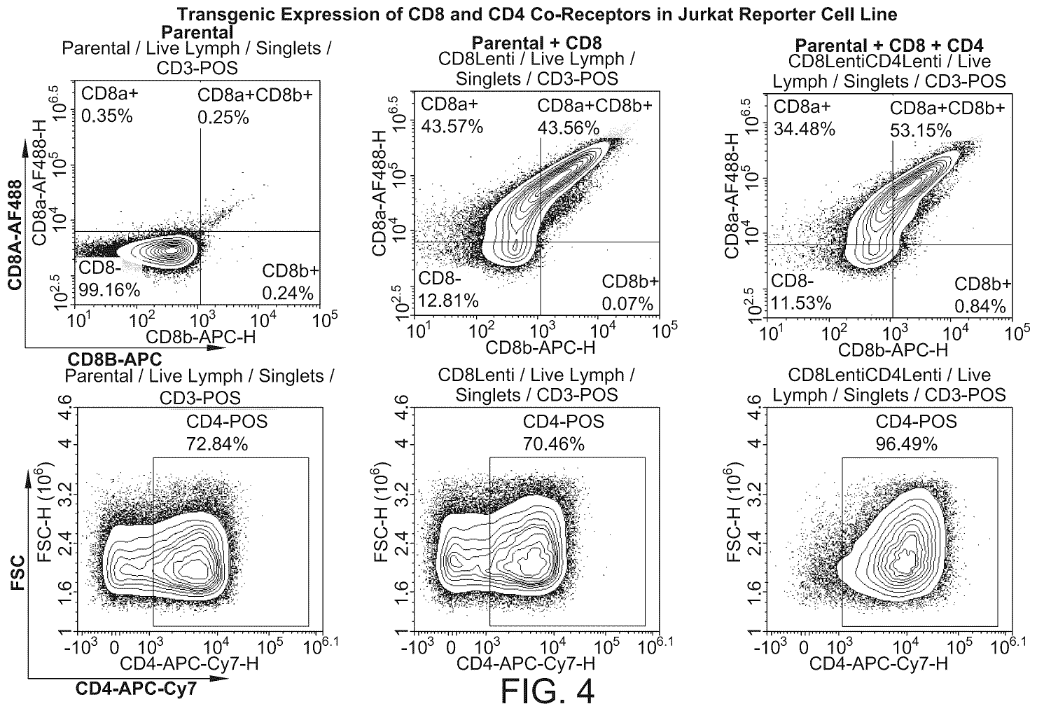

Lentivirus are prepared using HEK-293Ta cells and Jurkat NFAT cells are transduced. Jurkat NFAT cells are first transduced with CD8 Lentivirus and selected with 0.2 μg/ml puromycin to generate Jurkat NFAT_CD8Lenti cells. Subsequently, Jurkat NFAT_CD8Lenti cells are infected with CD4 Lentivirus and selected with 0.3 μg/ml puromycin. After 4 days selection with 0.3 μg/ml puromycin is adjusted back to 0.2 μg/ml for maintenance. Cells are harvested and stained with CD3, CD4, CD8A and CD8B. Jurkat NFAT parental cells are negative for CD8 (99.16% CD8 negative) within the CD3+ cell population. Results shown in FIG. 4 demonstrate that Jurkat LentiCD8 cells have 43.57% CD8A expression and 43.56% CD8A and CD8B double positive expression.

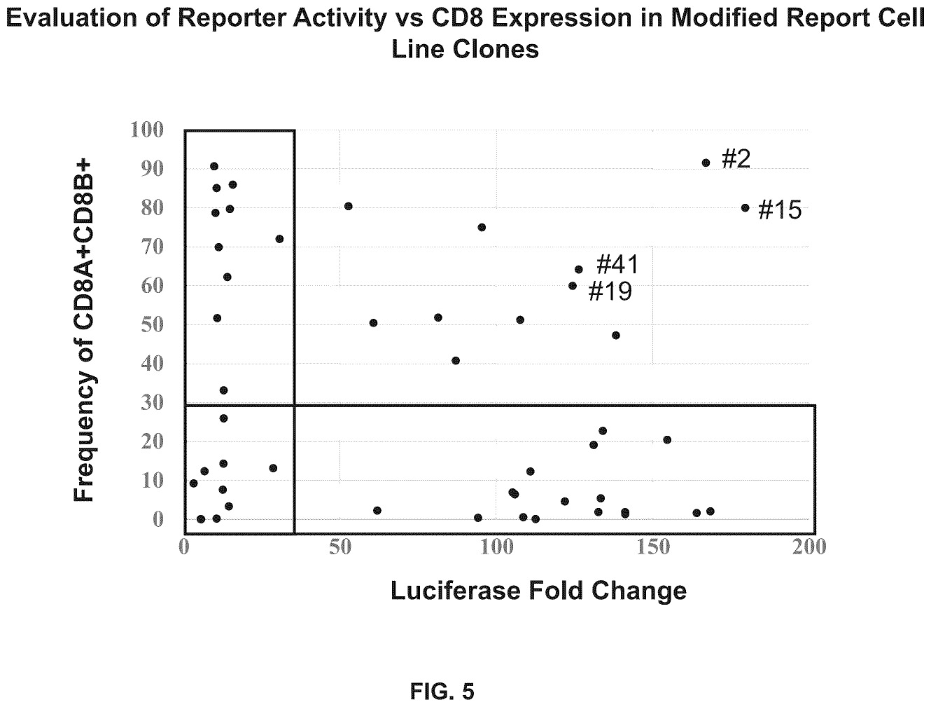

Single clones are generated from Jurkat NFAT_CD8Lenti pool. Peripheral Blood Mononuclear Cells (PBMCs) from 3 different donors are irradiated and seeded in 96-multiwell U bottom plates at 100k cells/well. Puromycin selected Jurkat NFAT_CD8Lenti stable pool cells are seeded at 0.5 cell/well on top of irradiated PBMCs to generate single clones. Single clones are cultured for one week with IL-2 (50 IU/ml) and phytohaemagglutinin (PHA; 0.25 μg/ml). During the second week cell medium is replaced with 100 IU/ml of IL-2. Grown back clones are evaluated for CD8A and CD8B expression and luciferase signal/noise ratio (PMA/Ionomycin vs untreated). Clones 2, 15, 19, 41 (>95% CD8 expression and >150 signal to noise ratio) are the best clones with higher CD8 expression and higher luciferase activity signal to noise ratio (FIG. 5).

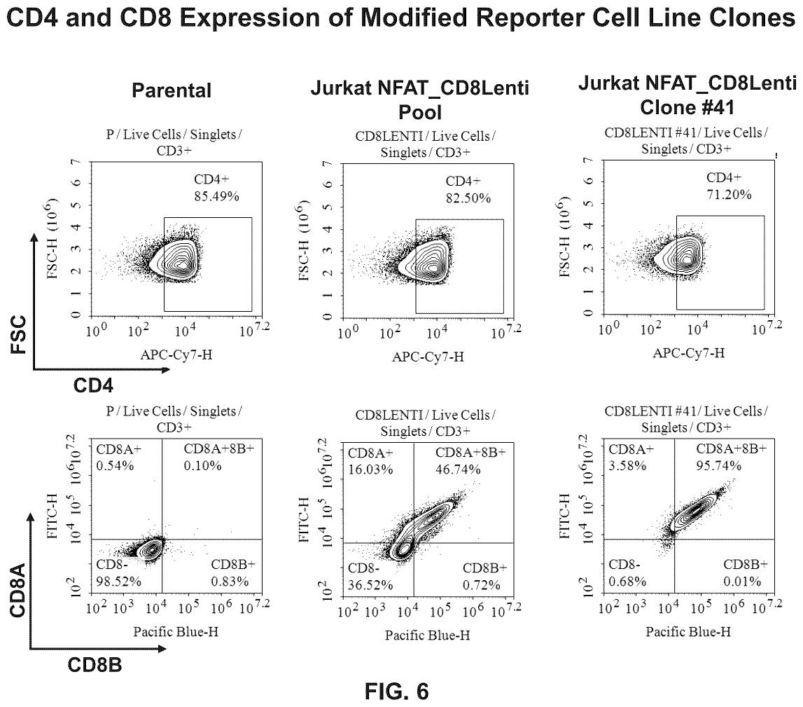

To better improve screening efficacy, clone #41 is selected from the Jurkat NFAT CD8Lenti pool. Flow cytometry analysis is performed to confirm the expression of CD8a and CD8b. Cells are stained with CD3, CD4, CD8A, and CD8B. As shown in FIG. 6, CD8A and CD8B double positive population is increased from 46.74% in the Jurkat NFAT_CD8Lenti pool to 95.74% in the #41 clone. This substantial increase of CD8 expression would allow us to capture better neoantigen reactive Class I TCRs. However, the CD4 expression was still not optimal.

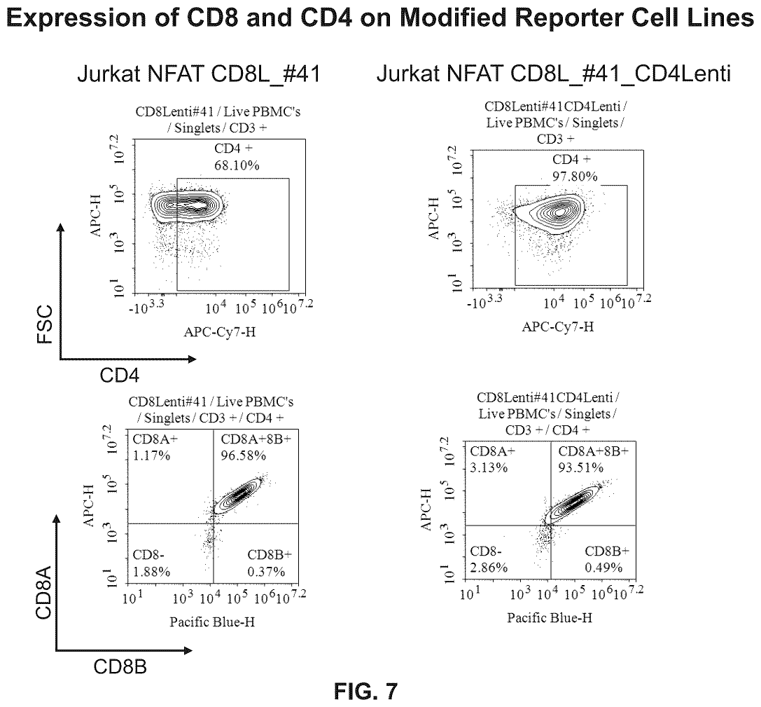

To improve the CD4 expression in Jurkat NFAT CD8Lenti #41, the cells are infected with lentivirus (pGenLenti-CD4_IRES_Puro). Flow cytometry analysis is then performed to evaluate the expression of CD4 by these cells. Cells are stained with CD3, CD4, CD8a, and CD8b. As shown in FIG. 7, the CD4 positive population is increased from 68% to 97.8%. CD8 expression is not changed significantly. Now upgraded #41 clone is both high CD8 and CD4 which improves TCR screening sensitivity.

### 2.2.2 Reporter Activity Time Course

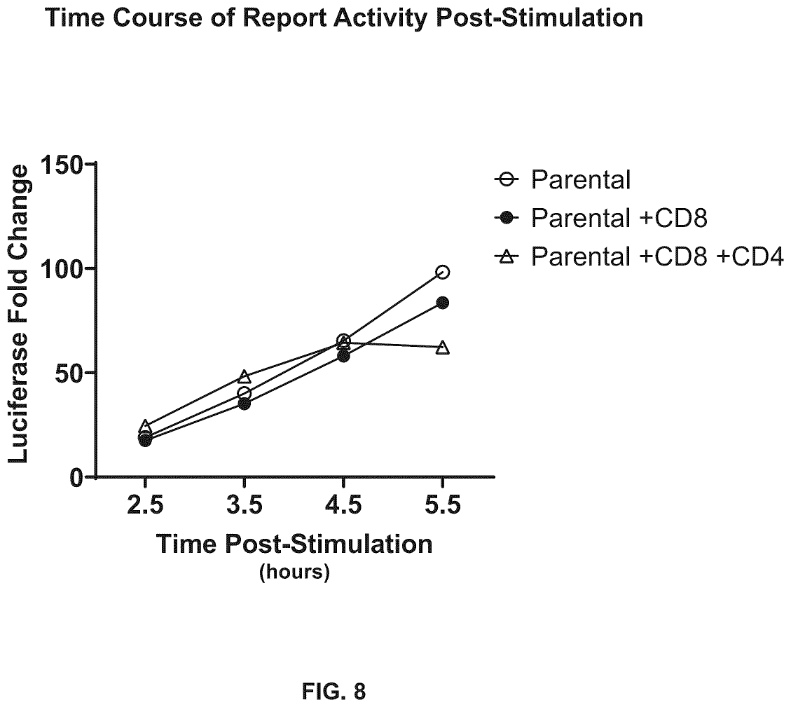

A time course study is performed to determine the best time point to harvest the co-culture. Jurkat cells are seeded in RPMI complete medium at 200k cells/well in 96-multiwell plates. Cells are treated with 50 ng/ml PMA and 1 μg/ml Ionomycin for 2.5, 3.5, 4.5 and 5.5 hrs. Cells are harvested and lysed with passive lysis buffer (Promega) at room temperature for 15 minutes. 50 μls of cell lysis is mixed with 100 μl of luciferase substrate (Promega). Luciferase signal intensities are detected with Luminometer. Luciferase activity folds changes are calculated by dividing PMA/Ionomycin treated condition to vehicle control treated conditions. As shown in FIG. 5, 4-5 hours is the best time to harvest cells since luciferase signals start to drop for the CD8Lenti_CD4Lenti pool. Data is shown in FIG. 8.

### 2.3 Optimization of Transfection Conditions in COS-7 Cells

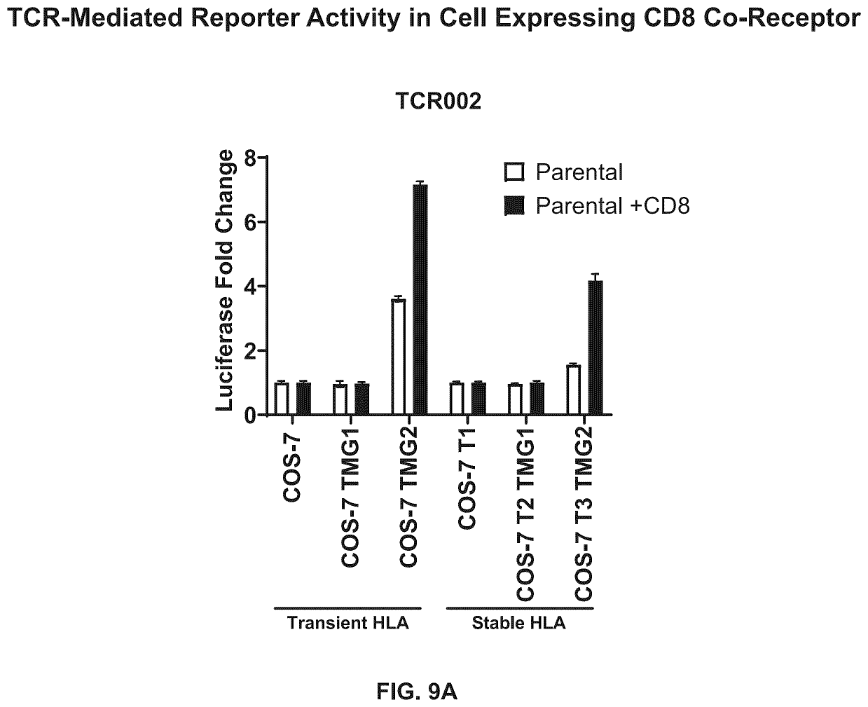

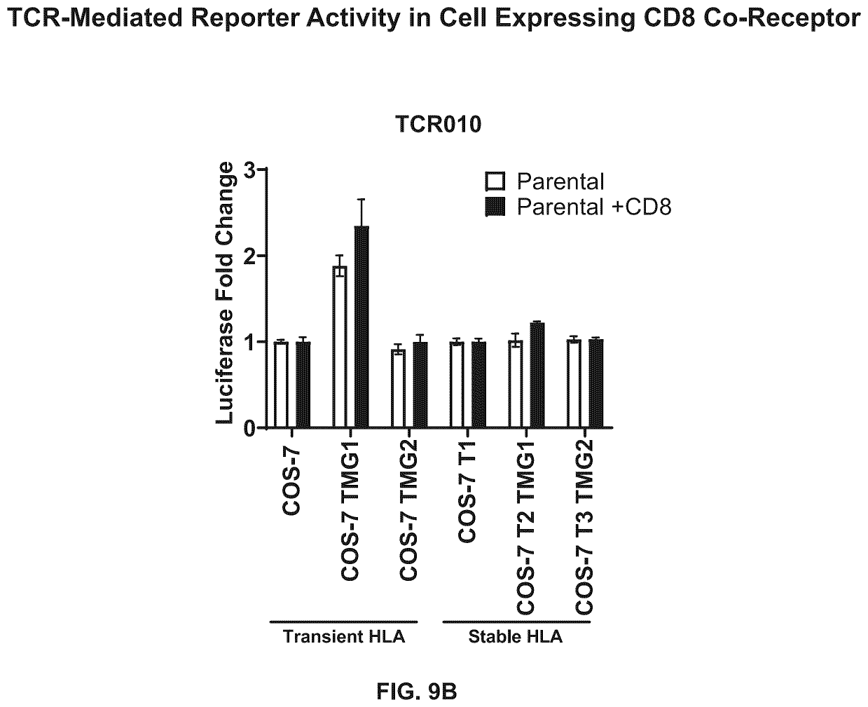

Day 1: COS-7 cells are seeded at 20,000 per well overnight in 96 multiwell plates. Day 2: COS-7 cells are transfected in each well with 150 ng of TMG1 or TMG2 and 75 ng of HLA A*11:01 and 75 ng of HLA A*02:01. Day 3: NEON transfection system is set up the following day and 5 million cells are electroporated with either TCR002 or TCR010 monkey-TCR (mTCR). Day 4: Jurkat cells are harvested and seeded on top of either transfected COS-7 cells or COS-7 cells stably expressing HLA A*11:01 or HLA A*02:01. After 5 hours co-culture, cells are harvested, and luciferase activity is measured. As shown in FIG. 9A, TCR002 TCR electroporated cells specifically recognized TMG2 (contained KRAS G12V mutation). As shown in FIG. 9B TCR010 TCR specifically recognized TMG-1 (contained R175H mutation) transfected COS-7 cells as expected. Transient transfection works better than stable pools in both TCR002 and TCR010 TCRs. In addition, Jurkat NFAT CD8Lenti has higher fold induction compared with Jurkat NFAT parental cells in both TCR co-culture experiments demonstrating the relevance of overexpressing CD8 in Jurkat NFAT cells for Class I TCRs.

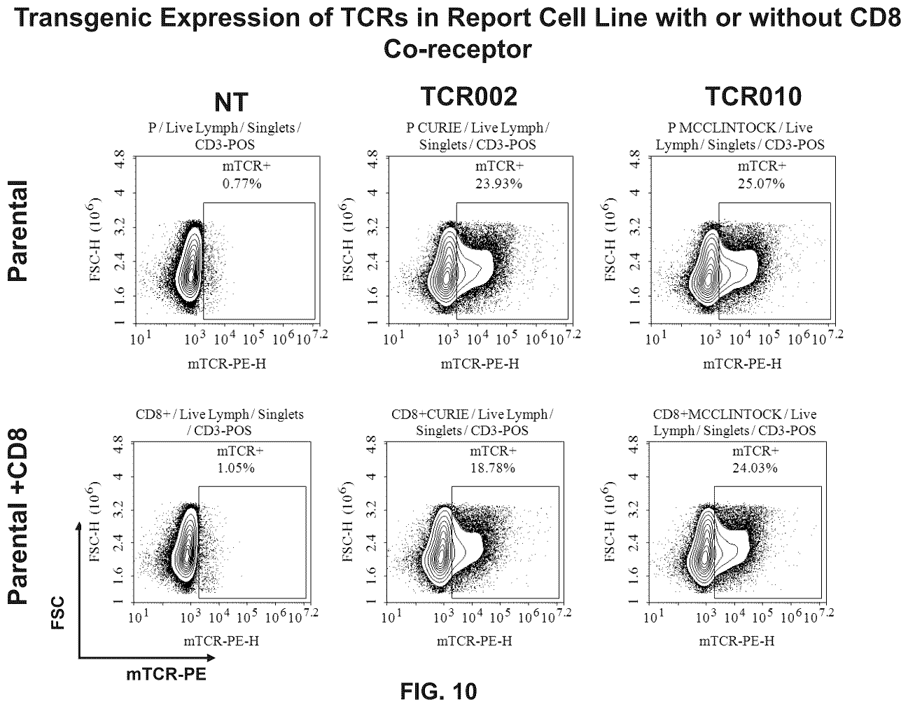

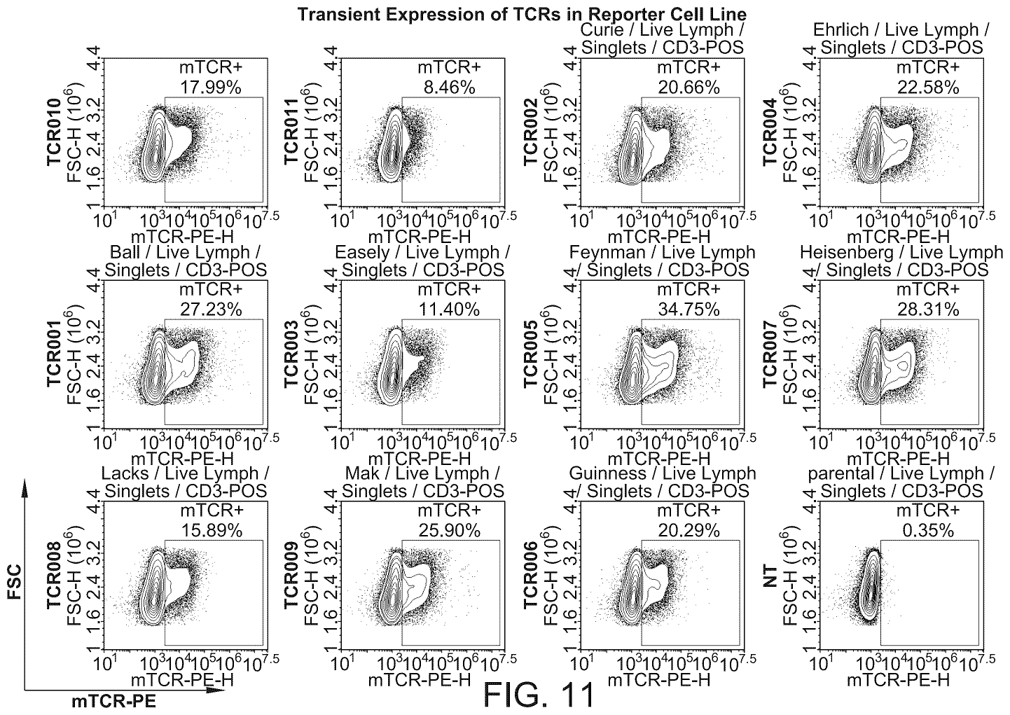

Jurkat NFAT electroporated with TCR002, TCR010 cells are analyzed using flow cytometry to detect the percentage of cells with mTCR expression. Cells are stained with CD3, CD4, CD8a, CD8b and mTCR antibodies. As shown in FIG. 10, cells express similar level of mTCR in Jurkat NFAT CD8Lenti cells compared with Jurkat NFAT parental cells. Over 90% of cells are viable in all six cell lines on the next day after electroporation suggesting the NEON electroporation system could provide highly viable T cells with sufficient percentage of mTCR expression (˜20%). This would allow co-culture experiments to be performed the next day without wasting time to recover cells. CD8 co-receptor expression did not improve TCR expression therefore suggesting that the addition of CD8 improved the TCR-peptide:MHC interaction to improve the reporter activity.

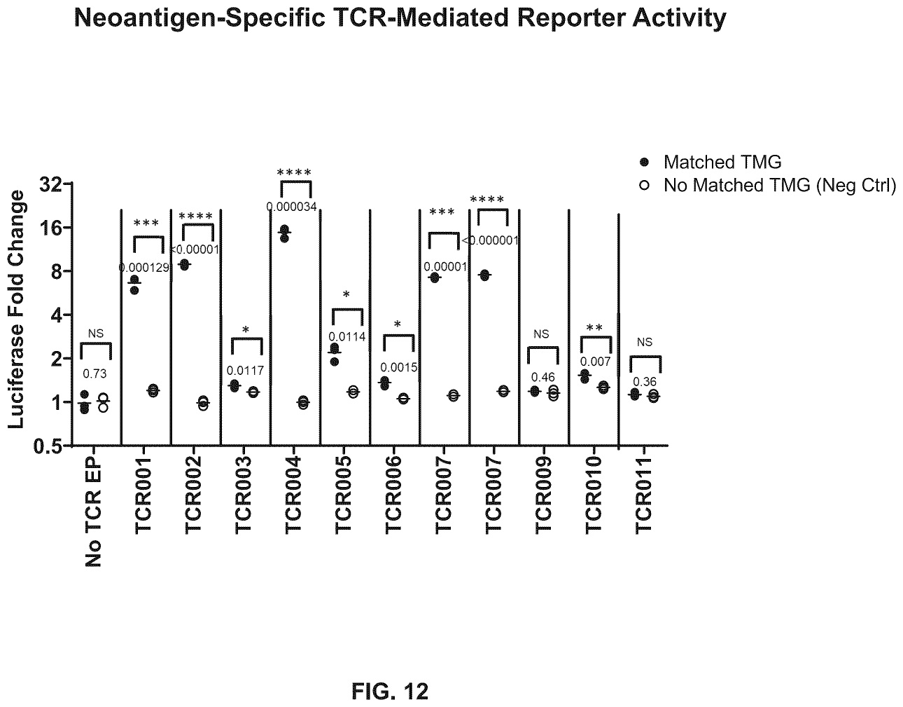

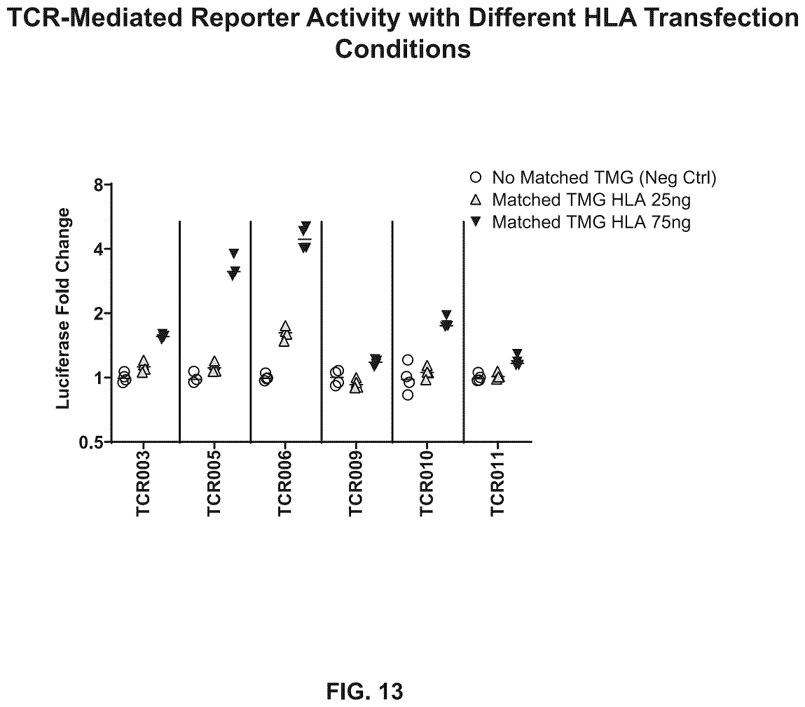

To examinate the reliability of JNR/COS co-culture system, several exemplary TCRs were tested. Flow cytometryanalyses were performed to evaluate the mTCR expression level in 11 TCRs, and cells are stained with CD3, CD4, CD8a, CD8b and mTCR antibodies. As shown in FIG. 11, mTCR expression varied from 8-35% (9 of 11 TCRs expressed above 15%) when cells are gated on CD3+. Day 1: COS-7 cells are seeded at 20,000 per well overnight in 96 multiwells. Day 2: COS-7 cells are transfected in each well with 150 ng of TMGs and 150 ng of HLAs (each 25 ng). Day 3: NEON transfection system is set up the following day and 5 million cells are electroporated with each TCR plasmid. Day 4: Jurkat cells are harvested and seeded on top of transfected COS-7 cells. After 5 hours of co-culture, cells are harvested, and luciferase activity is measured. Luciferase activity fold change (FC) is calculated based on cells without electroporation using TCR. TCR specific HLAs and matched neoantigens or TMGs are listed on the table below. (FIG. 12) Based on the statistical analysis, 9 of 11 TCRs from TCR library are confirmed with specificity against matched TMGs (i.e., a match TMG contained mutations specific to the TCR). No matched TMGs are irrelevant TMGs where no specific mutations are contained in the plasmid to serve as negative control. To troubleshoot the TCRs with low reactivity experiments are designed by transfecting different amounts of HLA plasmids. As you could see from FIG. 13, the signal to noise ratio is significantly increased when COS-7 cells are transfected with 75 ng of plasmids compared with 25 ng. This has been observed in all 6 TCRs which show relatively low reactivity based on FIG. 12.

### 2.4 Optimization of Peptide Pulsing Conditions in COS-7 Cells

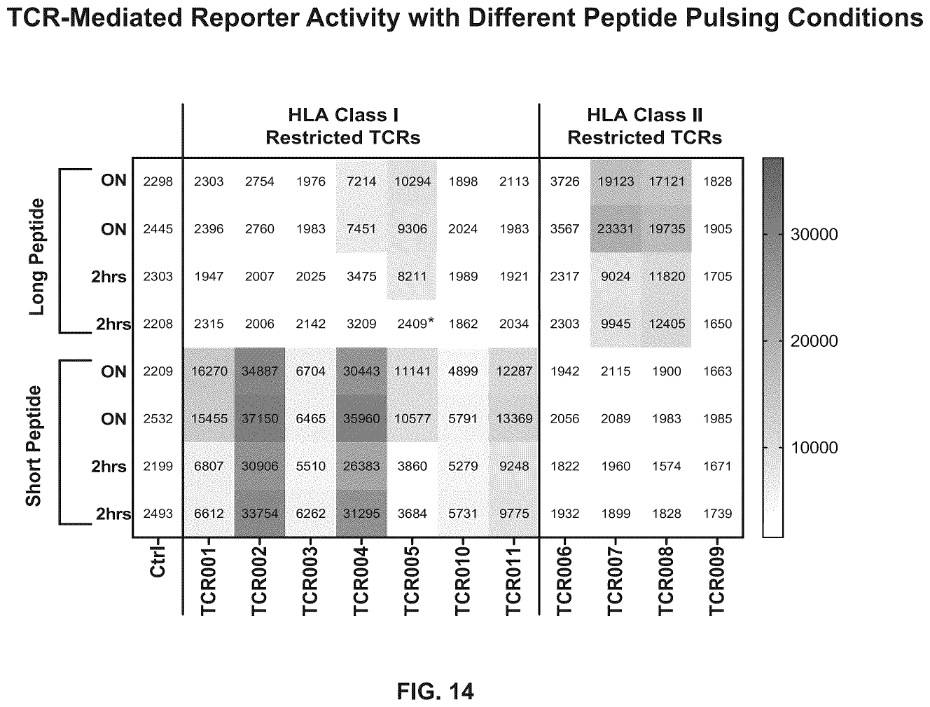

Peptide pulsing is tested with certain TCRs. COS-7 cells are pulsed with peptides either overnight or for 2 hours. Long peptides, as well as short peptides are used. 11 TCRs are electroporated for optimization studies. As shown in FIG. 14, three of 7 class I TCR are able to detect long peptide; however, all of the 7 class I TCRs are also able to react to short peptides. In addition, 3 of 4 Class II TCRs are reactive more to long peptides but not short peptides. In conclusion, overnight pulsing of peptide showed stronger signal compared with 2 hours. Class I TCRs recognize short peptide better and Class II TCRs recognize longer peptide better. The COS-7 peptide pulsing worked with most of the TCRs tested which demonstrates that COS-7 cells can be used to identify specific neoantigens in the reactive TMGs.

### 2.5 Development of Assay Controls

### 2.5.1 Anti-TCR Coated Plate Positive Control

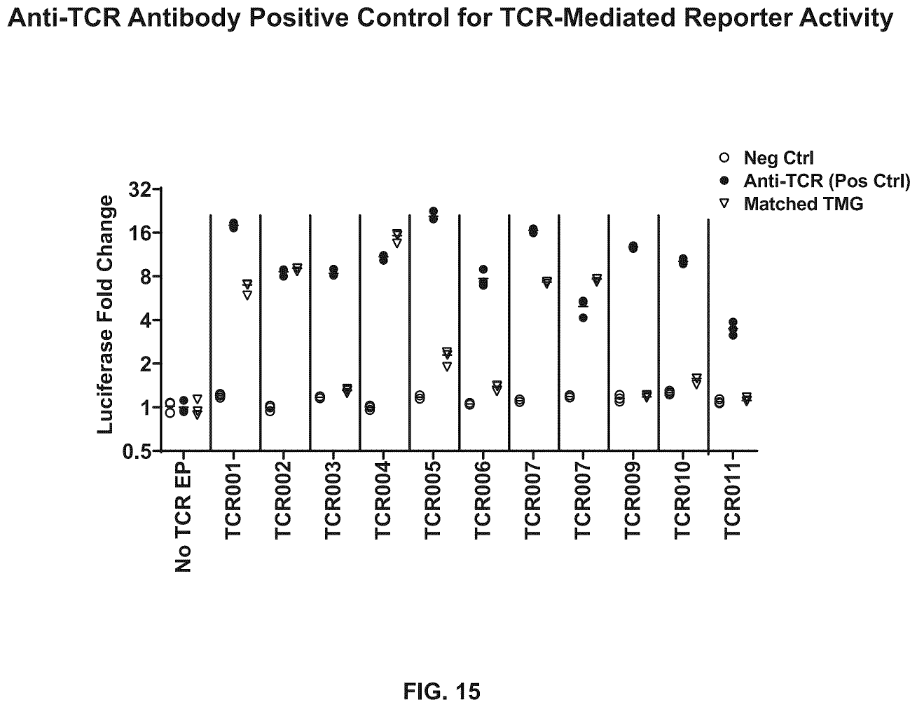

On the day of electroporation, H57 antibody is coated on the 96 multiwell plate overnight at 4° C. as a positive control. On the next day, Jurkat cells are seeded on the plate for 5 hours. Luciferase activity fold change (FC) is calculated based on cells without electroporation using TCR. Some of the TCRs demonstrated comparable levels of activation as H57 such as TCR002, TCR004, TCR001, TCR007 and TCR008 (FIG. 15). Some TCRs including TCR011, TCR009 and TCR006 are not activated as much with matched TMG they were with H57 coating suggesting that the TCR is successfully electroporated, but not fully activated. This might be due to the sub-optimal formation of HLA-neoantigen-TCR complex.

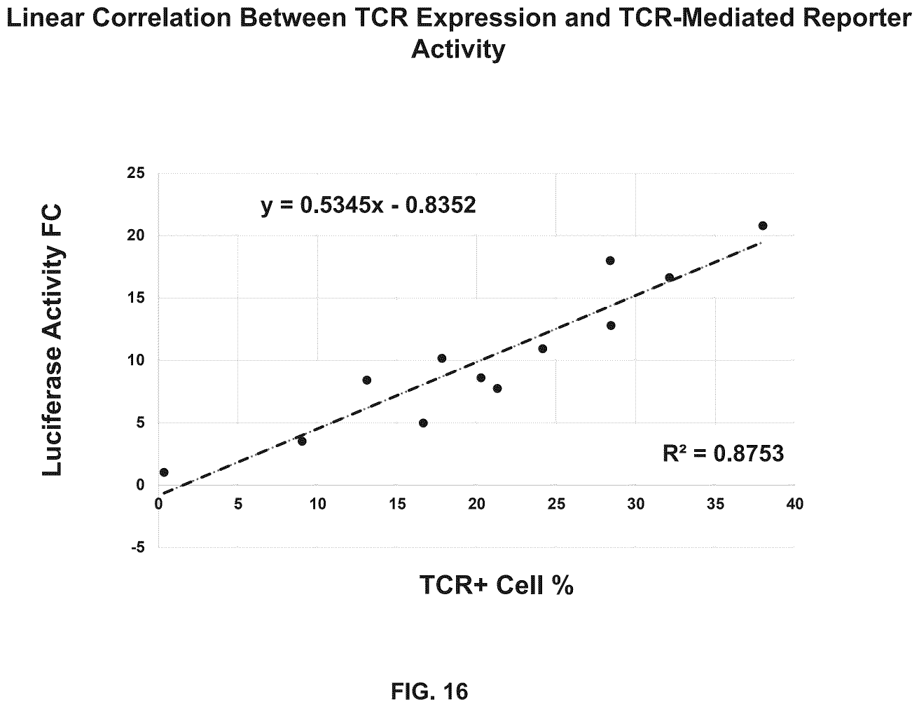

A scatter blot is generated using H57-coated Jurkat NFAT cells luciferase activity and mTCR expression based on the flow cytometry analysis. These cells are 12 cell lines shown in FIG. 16. Luciferase activity was positively associated with mTCR expression with R2 value of 0.8753 suggesting that luciferase activity from H57 coated plate could serve as optimal control besides flow cytometry for TCR expression and biological function.

### 2.6 Conclusion

The series of data described in this example illustrate the development of a method and cell lines that are used to screen TCRs isolated from primary T cells against various combinations of HLA and antigens. Optimal reporter activity is observed between 4-5 hours after stimulation. It is observed that addition of CD4 and CD8 co-receptors to the reporter cells improved TCR-mediated reporter activity. Isolation of a single CD8-modified report cell line clone, Clone #41 is achieved which improved the sensitivity of the assay to detect reactive TCRs. Development of an assay positive control, using plate-bound anti-TCR antibody, proved to be a robust control for functional TCR expression and correlated highly with the frequency of TCR expression measured by flow cytometry. Modulation of HLA plasmid amounts in the transfection reaction is found to improve the antigen-presentation and subsequent sensitivity of detecting reactive TCRs in this assay. Overall, the example illustrates the development and optimization of a high-throughput TCR screening platform to enable identification of TCR sequences, antigen-specificity, and HLA-restriction which could be used to identify novel therapeutic TCRs derived from primary tissues.

### Example 3: Patient 2599 TCR Screening

### 3.1 Mutation and HLA Calling

### 3.1.1 Sample Demographics

Patient 2599 is a male, colorectal cancer patient with the primary tumor located in the recto-sigmoid portion of the colon. At the time of collection, the patient's disease is Stage II-A. Patient 2599's tumor specimen is collected when the patient is 80 years old and prior to the start of treatment for the cancer diagnosis. A specimen of dissociated tumor cells (DTCs) from this patient is procured through a commercial vendor (Discovery Life Sciences; Huntsville, AL). A matched PBMC sample is also collected from the patient and used for the normal reference tissue.

### 3.1.2 Molecular Profiling Sample Processing

DNA and RNA are isolated from 1.2×10⁶ cells of a dissociated tumor sample and from 4.5×10⁶ of a matched PBMC sample. Quantification by fluorescence spectrometry indicated that yields are sufficient for downstream applications, and gel electrophoresis demonstrated an absence of degradation in the isolated genomic DNA. RNA is found to be of sufficient quality for paired end library preparation.

To assess somatic mutations, 100 ng tumor and 100 ng normal DNA are each processed through whole exome sequencing (WES) library preparation by way of hybrid capture. The final paired end libraries are sequenced on an Illumina NextSeqDx sequencer. Libraries are sequenced at 2×151 bp read lengths and yielded 2×420.16 M reads pass filter and 90.17% of non-index bases achieved >=Q30 quality score. Reads are subject to on-board demultiplexing to yield paired FASTQ files.

To assess the gene expression of transcripts of interest, 50 ng of RNA isolated from the dissociated tumor sample is processed through RNAseq library preparation by way of hybrid capture. The final paired end library is sequenced on an Illumina NextSeqDx sequencer at 2×74 bp read lengths. The sequencing run yields 2×462.71 M reads pass filter and 95.04% of non-index bases achieved >=Q30 quality score. Reads are subject to on-board demultiplexing to yield paired FASTQ files.

### 3.1.3 Molecular Profiling Analysis Pipeline

Raw reads from WES experiments are aligned to the human hg19 reference genome using BWA to create BAM files. Duplicate reads (paired reads mapped to identical locations of the genome) are discarded to avoid enrichment bias from PCR overamplification. To improve mapping quality, indel realignment and base quality recalibration are performed on BAM files.

Somatic mutation calling is performed on the tumor WES data using the normal WES data as the reference sequence. The mutation detection algorithms Mutect, MuSE, Varscan2, Mutect2, and Strelka are used to detect SNVs, the latter three are used to detect indels, and Mutect2 is used to detect MNVs. An SNV is only reported if it is detected by at least three of the five algorithms. An indel is reported if it is detected by at least one algorithm.

The detected mutations are annotated with ANNOVA and VEP. Only mutations meeting the following criteria are included in the final report: allele frequency (AF)>0.05 in the tumor sample; coverage at that position of at least 20 reads in the tumor sample and 10 reads in the normal sample; normal sample AF<0.02. The resulting mutations are filtered further to include SNVs and indels that are deemed to be non-synonymous to generate a final list of mutations.

Potential neoantigens are predicted for each mutation. An in silico strand representing a mutant peptide of up to 25 amino acid residues are derived, given that antigen lengths in human cells range from 8 to 25 bases. For every non-synonymous SNV, MNV, and in-frame indel, the in silico strand sequence is initiated 12 amino acid residues upstream of the mutated residue and ended 12 amino acid residues downstream of the mutated residue. For frameshift indels that resulted in a variant more than one residue in length, amino acids are included in the in silico strand until a stop codon is detected in the new reading frame. If multiple transcripts are known to overlap the somatic mutation position, an in silico strand is derived for each such transcript and all unique strands are reported for each somatic mutation.

Class I and II HLA alleles are derived from WES data. Optitype, Polysolver and HLAVBSeq are applied to infer class I HLA alleles at two-field/four-digit resolution (e.g., HLA-A*02:01). HLAVBSeq is applied to infer class II HLAs. The in silico strand peptide sequences and the HLA types are input together to netMHCpan4.1 to predict potential interactions.

Bulk RNA-Seq data is analyzed to quantify the expression level of each gene in the tumor sample. Reads from FASTQ are aligned to the hg19 genome using STAR with the two-step procedure. Cufflinks are applied to the resulting BAM files to calculate the Fragments

Per Kilobase of transcript per Million mapped reads (FPKM) value of each gene. FPKM values are converted to deciles to represent ten gene expression levels. Gene expression values corresponding to each mutated gene are reported alongside the detected mutations from WES.

### 3.1.4 Molecular Profiling Results

WES analysis revealed 73 somatic non-synonymous mutations ranging in allele frequencies from 0.058 to 0.309 and gene expression values ranging from 0.6 to 48.5 FPKM. The mutations produced 76 unique in silico strands up to 25 residues in length. Of the 76 unique fragments, one is wholly contained within another in silico strand and removed from further processing.

### 3.2 Design and Construction of Synthetic Reagents

### 3.2.1 Neoantigen Reagent Design

To create peptide fragments containing the patient's somatic mutations, a total of 75 in silico strands representing non-synonymous mutations are synthesized as peptides with crude quality. To create vectors containing the same somatic mutations in nucleic acid form, the 75 amino acid sequences are reverse translated in silico and codon optimized for expression in human cells. A total of 5 tandem minigenes (TMGs) are designed by concatenating a set of 15 such amino acid sequences into one open reading frame. Incidental BamHI, EcoRI, NotI and NheI sites are removed by replacing codons within the restriction sites with synonymous codons. The nucleotide sequence GAG AAT TCG (codes for Glu (E)/Asn (N)/Ser(S) and contains an EcoRI restriction site) is added to the 5′ end of each TMG gene, and the nucleotide sequence AAG GAT CCC (codes for Lys (K)/Asp (D)/Pro (P) and has a BamHI restriction site) is added to the 3′ end of each TMG gene.

### 3.2.2 TMG Plasmid Synthesis

Each TMG, together with the flanking restriction sites, is synthesized and cloned into the masterTMG_pcDNA3.1 (+) plasmid in frame with existing start and stop codons using EcoRI (5′) and BamHI (3′) restriction enzymes. Each final plasmid is prepared in TE with 95%+5% supercoiled plasmid and ≤0.005 EU/μg endotoxin content.

### 3.2.3 Human Leukocyte Antigen (HLA) Plasmid Assembly

Peptide sequences for each HLA allele found in the patient sample are retrieved from the IPD-IMGT/HLA Database (ebi.ac.uk). Each peptide sequence is reverse translated in silico and codon optimized for expression in human cells. A BamHI restriction site and a Kozak site are added at the 5′ end of the coding sequence and an EcoRI restriction site and translational stop codon are appended to the 3′ end. The assembled sequence is synthesized and cloned into pcDNA3.1 (+) using BamHI and EcoRI restriction enzymes. The resulting plasmids are prepared in TE with 95%+5% supercoiled plasmid and ≤0.005 EU/μg endotoxin content.

3.3 Single-cell RNA Sequencing (scRNAseq) Analysis of TILs from Dissociated Tumor Sample

### 3.3.1 Cell Preparation

Patient 2599 DTCs are thawed, washed, and prepared in a single cell suspension. Cells are counted using an NC3000 automated cell counter (Chemometec). Cell viability is 83.8% and a final concentration of 400 cells/μL. The single cells suspension is loaded on a Chromium Controller (10× Genomics) with a targeted cell recovery of 8,000 cells.

### 3.3.2 Single-cell Paired End Library Preparation and Sequencing

Prepared single cell suspensions are processed to distribute single cells into partitions using the 10× Chromium instrument. The resulting single-cell emulsion is processed to yield cDNA. The cDNA library is used as input to prepare a gene expression paired end library (GEX) and a TCR-specific paired end library (VDJ). The final paired end libraries are combined and loaded onto an Illumina NextSeqDx sequencer. Libraries are sequenced at 26+10+10+122 bp read lengths. The sequencing run yields 2×637.72 M reads pass filter and 81.91% of non-index bases achieved >=Q30 quality score.

3.3.3 scRNAseq Analysis

VDJ sequencing data are preprocessed using the CellRanger toolkit (version 5.1) provided by 10× Genomics. Raw BCL files are converted to FASTQ files. Raw V(D)J sequencing reads are assembled into contigs using a graph-based algorithm with the aid of the pre-built reference sequence from the IMGT (www.imgt.org) database. Cells with identical productive V(D)J transcripts are considered to belong to the same clonotype. The following are reported for each unique clonotype: the amino acid sequence of the CDR3 region, the full-length FASTA sequence of the TRA chain, the full-length FASTA sequence of the TRB chain, and the clonotype frequency, defined as the number of cells in which each clonotype is observed.

### 3.4 TCR Reconstruction

### 3.4.1 T-Cell Receptor (TCR) Assembly

Single-cell RNAseq analysis yields 423 clonotypes where 281 clonotypes contained exactly one beta chain and one α chain. Clonotype frequency ranged from 1 to 28 cells with 48 clonotypes observed in more than one cell and 1 clonotype observed in more than 10 cells.

All clonotypes present in 3 or more cells and containing both an α and a beta chain are modified and assembled to create TCRs for a total of 18 TCRs. Each raw beta chain sequence is modified by replacing all sequence 5′ of the start of the V region with an NheI restriction site, and the entire constant region is replaced with a BspI restriction site. Each α chain was modified by the replacement of all sequence 5′ of the start of the V region with an XmaI restriction site, and the constant region is replaced with a SacII restriction site.

Rare codons (defined as codons used <10% according to the Homo sapiens codon usage table) are replaced with more frequently used synonymous codons throughout the beta and a gene open reading frames. Incidental NheI, BspI, XmaI, and SacII restriction sites are eliminated from the open reading frame by replacing bases within the restriction sites with synonymous codons not found within each restriction site.

### 3.4.2 TCR Plasmid Assembly

Each α and β gene is synthesized independently and subcloned into pZT2 using the synthesized restriction sites (NheI and BspEI for the beta gene and XmaI and SacII for the α gene). Each final plasmid is prepared in 10 mM Tris-HCl, pH 8.0, 1 mM EDTA (TE) with 95%+5% supercoiled plasmid and ≤0.005 EU/μg endotoxin content.

### 3.5 Patient 2599 TCR Screening

### 3.5.1 Experimental Design and Methods

Day 1: COS-7 cells are seeded at 20,000 per well overnight in 96 multiwell plates. Day 2: COS-7 cells are transfected in each well with 150 ng of TMGs+300 ng of HLAS (75 ng each). Day 3: NEON transfection system is set up the following day and 5 million cells are electroporated with each of the 18 TCR plasmid and negative control (NTC). Day 4: Jurkat cells are harvested and seeded on top of transfected COS-7 cells. After 5 hours co-culture, cells are harvested, and luciferase activity is measured. There are 5 TMGs designed for the relevant mutations, and 18 TCRs are picked from 10× single cell sequencing for patient 2599. In addition, patient 2599 has 2 HLA-A, 2 HLA-B, 2 HLA-C, 2 HLA-DQ-A, 2 HLA-DQ-B, 1 DP-A, 1 DP-B, 2 DRB1 and 2 DRB3 (these two are screened later). HLA plasmids are separated into 4 groups (HLA A&B, HLA C, HLA DQ and HLA DP&DR) to reduce the number of combinations with TMG plasmids. On the day of electroporation, H57 antibody is coated on the 96 multiwell plate overnight at 4° C. as positive control. The next day, Jurkat cells are seeded on the plate for 5 hours. Luciferase activity fold change (FC) is calculated based on cells seeded without H57 coating. All cells with electroporated TCRs show higher luciferase activity in H57 coated condition suggesting that TCRs are biologically functional.

### 3.5.2 Screening Results

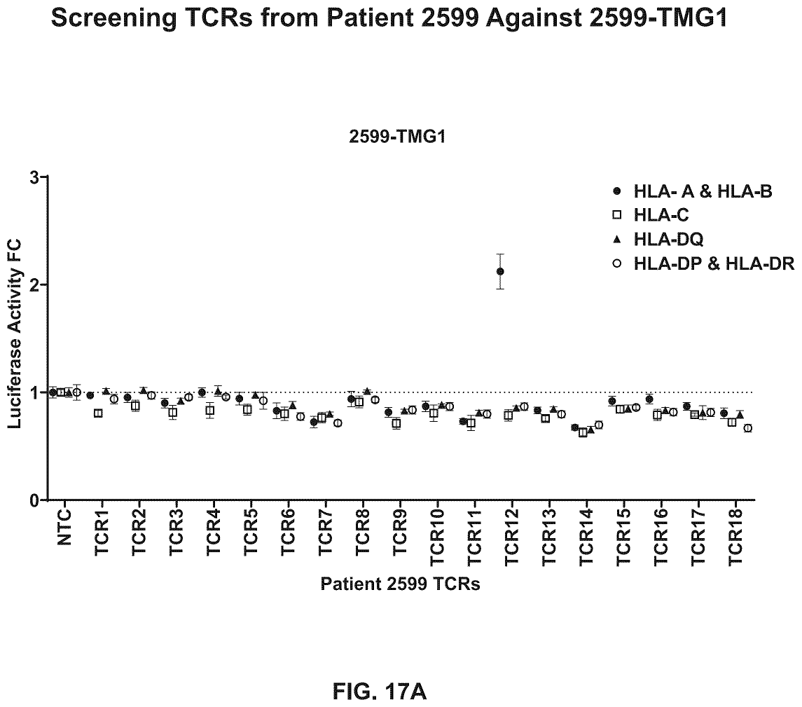

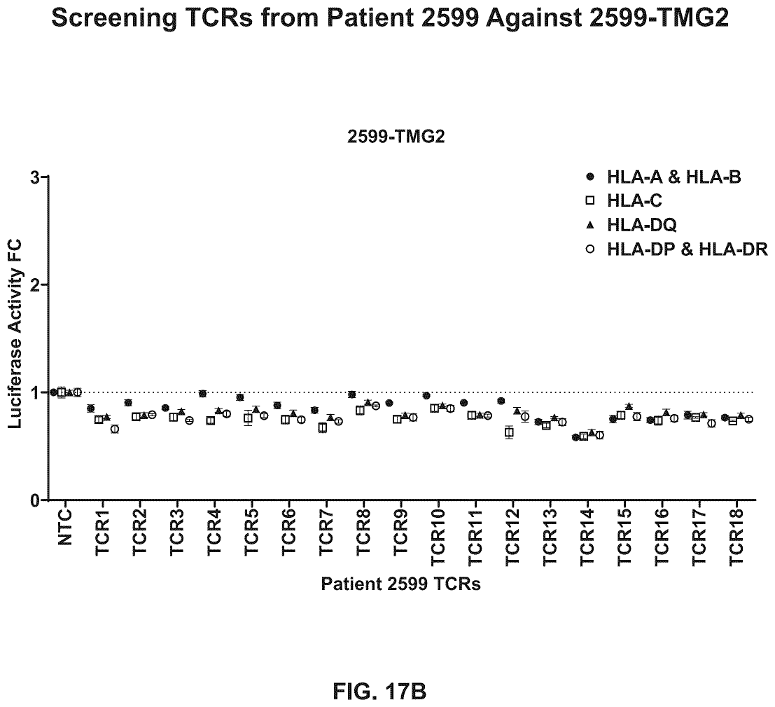

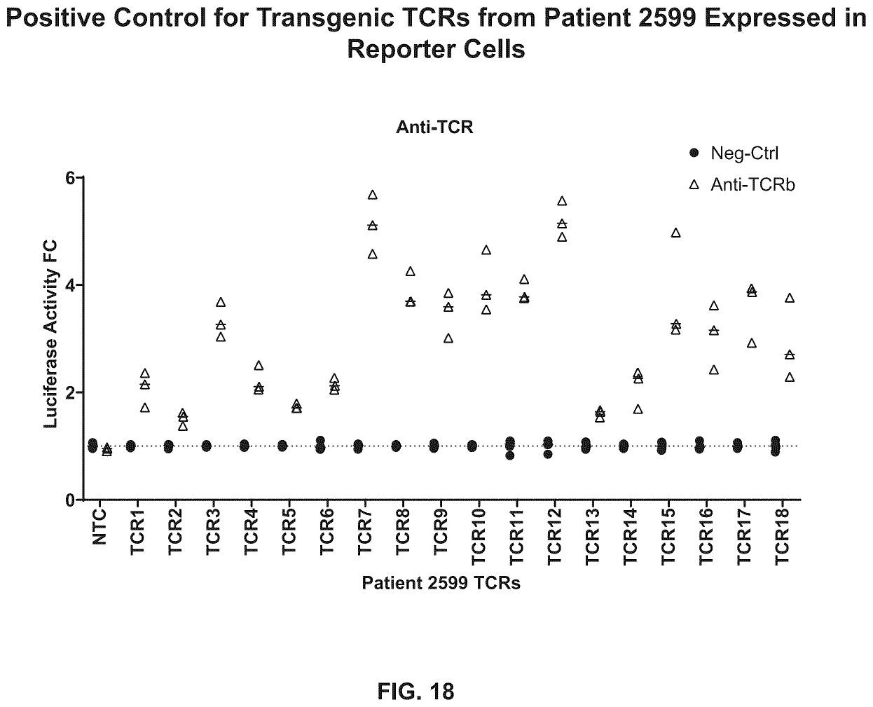

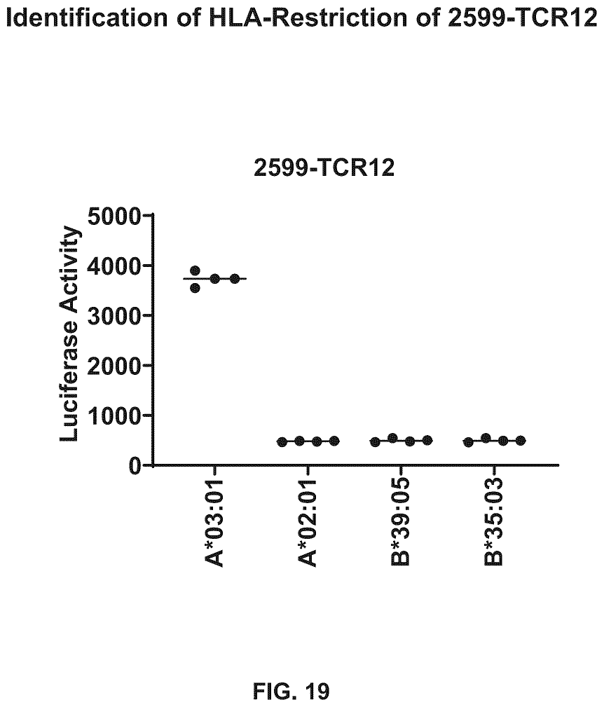

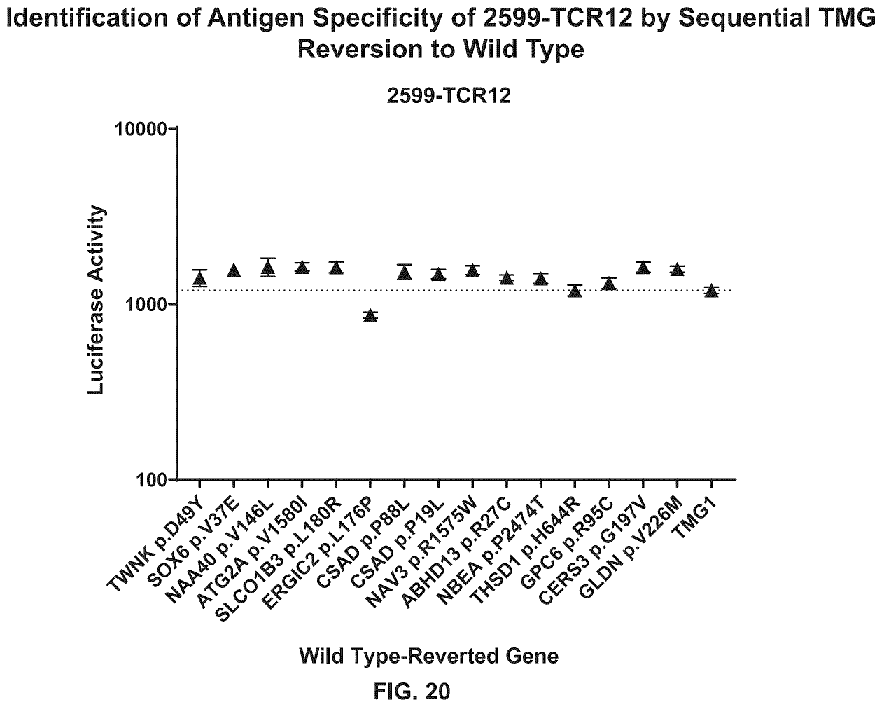

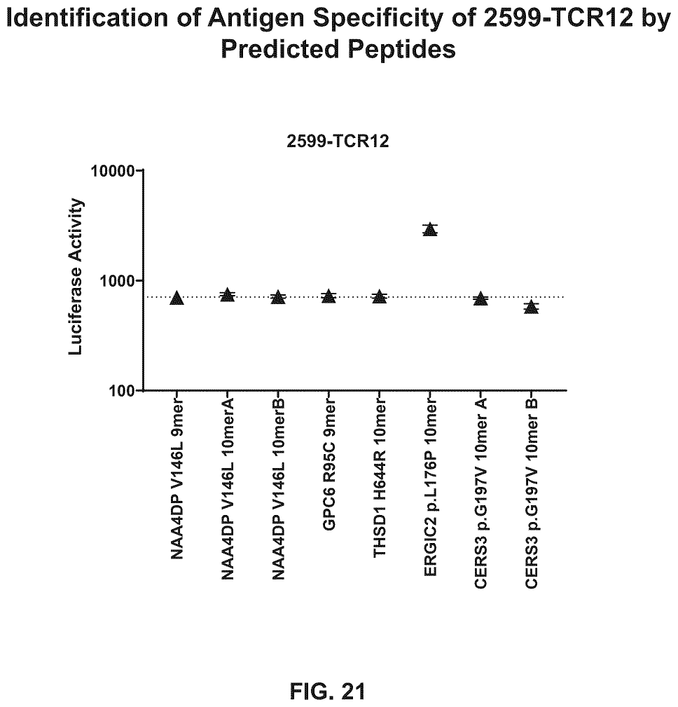

As shown in FIG. 17A-B, TCR 12 is specific to the combination of TMG1 and an HLA allele in either locus HLA-A or HLA-B, but not TMG2 combined with the same set to HLAs. All patient 2559 TCRs screened are capable of inducing reporter activity when cross-linked with an anti-TCR antibody, verifying that every TCR is expressed and therefore apparently non-reactive TCRs are not the result of non-expression (FIG. 18). To further define the HLA allele specificity, COS-7 cells are transfected with individual HLA allele plasmids and TMG1. The results show that HLA-A*03:01 is the specific HLA restricting 2599-TCR12 (FIG. 19). To determine which mutation in TMG1 is being recognized by 2599-TCR12, a reversion TMG is designed for each mutation represented in TMG1, where each reversion TMG is identical to TMG1 except for one mutation that is reverted to its wildtype allele. The reversion TMG containing a wildtype form of the ERGIC2 p.L176P has lower luciferase activity after co-culture, suggesting that ERGIC2 p.L176P plays a critical role in 2599-TCR12 reactivity (FIG. 20). To further define the minimal epitope of 2599-TCR12, an online peptide prediction tool predicted potential candidates with minimal residue of peptide likely to bind with HLA-A*03:01. The 25-mer peptide does not work for 2599-TCR12 specificity test since some Class I TCRs do not work with long peptides. As shown in FIG. 21, a ERGIC2 p.L176P 10-mer is specific to 2599-TCR12, confirming TMG reversion data.

### 3.6 Conclusion

The series of data described in this example illustrate the application of a high-throughput TCR isolation and screening method in a patient derived tumor specimen. Using a dissociated tumor sample from colorectal cancer Patient 2599, paired TCRα/β sequences are identified from tumor infiltrating T cells. These paired TCR sequences are reconstructed in silico from which DNA expression vectors encoding eighteen TCRs from Patient 2599 are generated. Using the TCR screening method, all eighteen TCRs are successfully screened and one TCR, 2599-TCR12 is found to be specific for the ERGIC2 p.L176P neoantigen when presented in the context of HLA-A*03:01. Overall, these data demonstrate a process by which neoantigen-specific TCRs can be identified and functionally validated using a high-throughput TCR screening method. This method is used to identify potentially therapeutic TCRs.

### Example 4: Patient 8434 TCR Screening

### 4.1 Mutation and HLA Calling

### 4.1.1 Sample Demographics

Patient 8434 is a female, colorectal cancer patient. Patient 8434's tumor specimen is collected when the patient is 66 years old and prior to the start of treatment for the cancer diagnosis. A specimen of dissociated tumor cells (DTCs) from this patient is procured through a commercial vendor (Discovery Life Sciences; Huntsville, AL). A matched PBMC sample collected from the patient is used for the normal reference tissue.

### 4.1.2 Molecular Profiling Sample Processing

DNA and RNA are isolated from each of a dissociated tumor sample and from a matched PBMC sample. Quantification by fluorescence spectrometry indicated that yields are sufficient for downstream applications, and gel electrophoresis demonstrated an absence of degradation in the isolated genomic DNA. RNA is found to be of sufficient quality for paired end library preparation.

To assess somatic mutations, 200 ng tumor and 200 ng normal DNA are each processed through whole exome sequencing library preparation by way of hybrid capture. The final paired end libraries are sequenced on an Illumina NextSeqDx sequencer. Libraries are sequenced at 2×101 bp read lengths and yielded 2×558.2 M reads pass filter and 92.21% of non-index bases achieved >=Q30 quality score. Reads are subject to on-board demultiplexing to yield paired FASTQ files.

To assess the gene expression of transcripts of interest, 50 ng of RNA isolated from the dissociated tumor sample is processed through RNAseq library preparation by way of hybrid capture. The final paired end library is sequenced on an Illumina NextSeqDx sequencer at 2×76 bp read lengths. The sequencing run yielded 2×443.7 M reads pass filter and 95.38% of non-index bases achieved >=Q30 quality score. Reads are subject to on-board demultiplexing to yield paired FASTQ files.

### 4.1.3 Molecular Profiling Analysis Pipeline

Bioinformatic analysis to profile somatic mutations and HLA alleles for sample 8434 is performed as described in Example 3.

### 4.1.4 Molecular Profiling Results

WES analysis revealed 106 somatic non-synonymous mutations ranging in allele frequencies from 0.055 to 0.745 and gene expression values ranging from 0 to 140.46 FPKM. The somatic mutations produce 111 unique in silico neoantigen candidates.

### 4.2 Synthetic Mutation Assembly

### 4.2.1 Neoantigen Reagent Design

To create peptide fragments containing the patient's somatic mutations, a total of 74 in silico neoantigen candidates representing non-synonymous mutations are synthesized with crude quality. To create vectors containing mutations in nucleic acid form, the 74 amino acid sequences are reverse translated in silico and codon optimized for expression in human cells. A total of 6 TMGs are designed by concatenating a set of either 12 or 13 sequences into one open reading frame as described in Example 3.

### 4.2.2 TMG Plasmid Synthesis

TMGs are synthesized as described in Example 3.

### 4.3 Human Leukocyte Antigen (HLA) Plasmid Assembly

Plasmids encoding the patient's HLA alleles are designed and synthesized as described in Example 3.

4.4 Single-Cell Analysis of TILs Sorted from Dissociated Tumor Sample

### 4.4.1 Cell Sorting and Preparation

Cells are washed as previously described in Example 3. A total of 10% of cells are set aside to grow TILs. The remaining cells are stained with anti-CD3 and anti-CD45 antibodies. CD3+CD45+ cells are sorted with a SONY SH800 cell sorter. Cells are subsequently washed with BSA 0.2% and resuspended in an appropriate volume of BSA 0.2%. Sorted cells are 88% viable (compared with 25% of unsorted cells) and prepared at a concentration of 650 cells/μL, enabling targeting of 10,000 cells in subsequent processing.

### 4.4.2 Single-Cell Paired End Library Preparation and Sequencing

The sorted tumor sample is processed to create paired end libraries and sequenced as described in Example 3. The sequencing run yielded 2×441.36 M reads pass filter and 90.7% of non-index bases achieved >=Q30 quality score.

4.4.3 scRNAseq Analysis

The GEX and VDJ sequencing data are preprocessed using the CellRanger toolkit (version 5.1) provided by 10× Genomics. The BCL files were converted to raw FASTQ files. The FASTQ files for the GEX and VDJ experiments are processed separately.

GEX reads realigned to the human GRCh38 reference genome. Cell barcode assignment and unique molecular identifier (UMI) counts are then performed to create a single-cell gene expression matrix. Doublets and cells with >10% mitochondrial gene counts are filtered out. Raw read counts are normalized and scaled using Seurat. Approximately 2,000 highly variable genes are identified using the FindVariableGenes module. Principal component analysis (PCA) and uniform manifold approximation and projection (UMAP) are performed for dimension reduction and a shared nearest neighbor (SNN) algorithm is applied to cluster the cells.

The raw V(D)J sequencing reads are assembled into contigs using a graph-based algorithm with the aid of the pre-built reference sequence from the IMGT (www.imgt.org) database. Cells with identical productive V(D)J transcripts are considered to belong to the same clonotype. The following are reported for each unique clonotype: the amino acid sequence of the CDR3 region, the full-length FASTA sequence of the TRA chain, the full-length FASTA sequence of the TRB chain, and the clonotype frequency, defined as the number of cells in which each clonotype is observed.

The number of detected cells, mean reads per cell, reads mapped to the genome etc. were reported by CellRanger as quality control measurements.

To ensure that every T cell in the study has both gene expression information and its TCR sequences, only cells detected in both GEX and V(D)J are used in the analysis.

### 4.5 TCR Reconstruction

### 4.5.1 T-Cell Receptor Assembly

Single-cell RNAseq analysis yields 1775 clonotypes where 1718 clonotypes contain exactly one beta chain and one α chain. Clonotype frequency ranges from 1 to 264 cells with 516 clonotypes observed in more than one cell and 72 clonotypes observed in 10 or more cells. From the most frequent 150 clonotypes, 36 α and β chain pairs are modified and assembled to create 36 individual TCRs. Alpha and β chains are modified as described in Example 3.

### 4.5.2 TCR Plasmid Assembly

Alpha and β chains are synthesized and cloned into TCR plasmids as described in Example 3.

### 4.6 In Vitro TCR Screening

### 4.6.1 Experimental Design and Methods

On day 1, COS-7 cells are seeded in 96 wells at 20,000 cells per well and incubated overnight at 37° C. On day 2, each well of COS-7 cells is transfected with 150 ng of TMG plasmids and 300 ng of HLA plasmids (75 ng per HLA allele). On day 3, five million cells are electroporated with each of the 18 TCR plasmid and a negative control (NTC). On day 4, Jurkat cells are harvested and seeded on top of transfected COS-7 cells. After 5 hours of co-culture, cells are harvested, and luciferase activity is measured. There are 6 TMGs designed for the relevant mutations, and 36 TCRs are picked from single cell sequencing for patient 8434. In addition, this patient has 2 HLA-A, 2 HLA-B, 2 HLA-C, 2 HLA-DQ-A, 2 HLA-DQ-B, 1 DP-A, 2 DP-B, 1 DRB1 and 1 DRB3 alleles. The HLA plasmids are segregated into 5 groups (HLA A&B, HLA B&C, HLA DP, HLA DQ and HLA DR) to reduce the number of combinations with TMG plasmids. On the day of electroporation, H57 antibody is coated on the 96 multiwell plate overnight at 4° C. as a positive control. On the next day, Jurkat cells are seeded on the plate for 5 hours. Luciferase activity fold change (FC) is calculated based on cells seeded without H57 coating. All cells with electroporated TCRs exhibited higher luciferase activity in H57 coated condition suggesting that the TCRs are biologically functional.

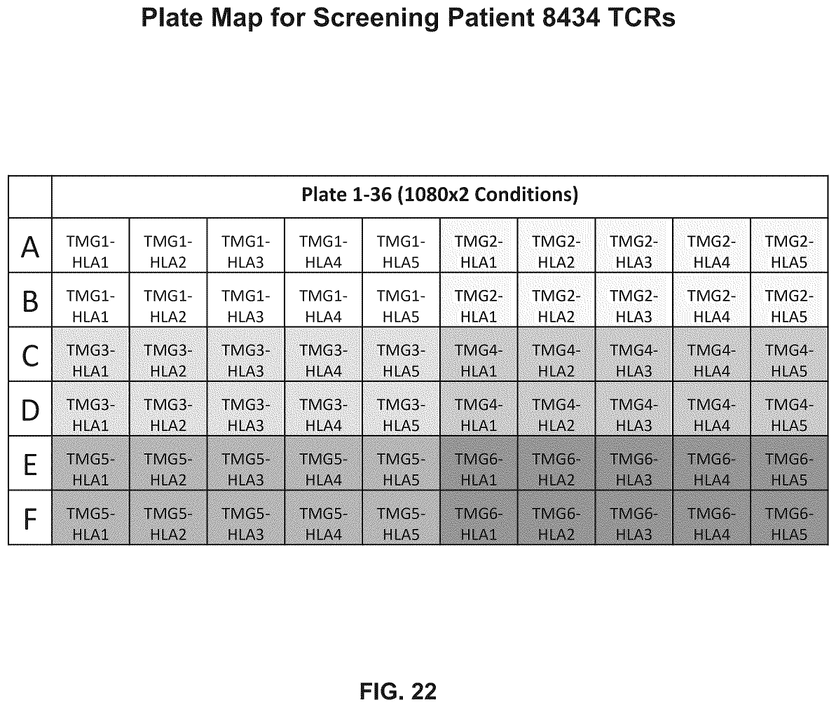

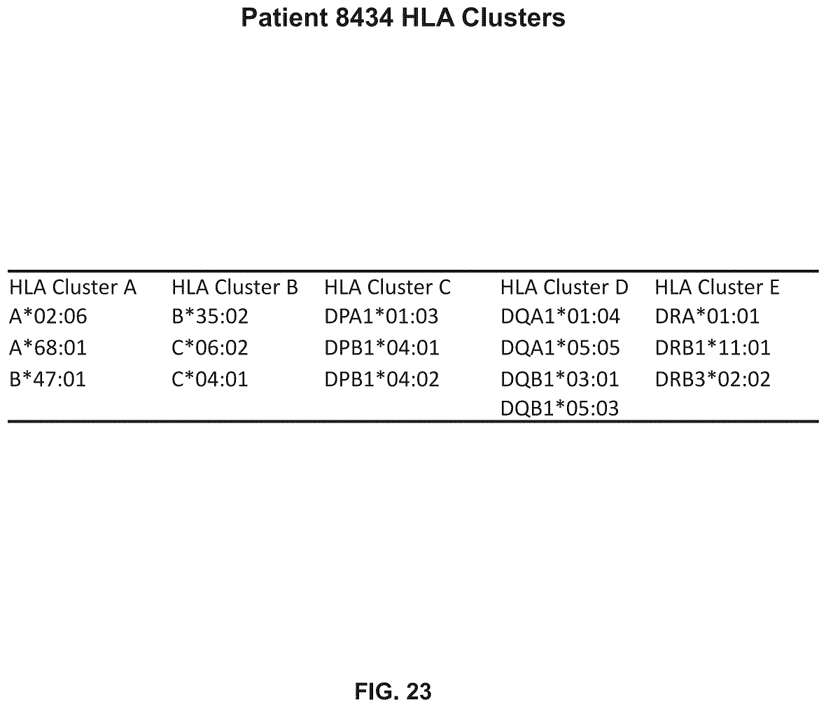

The plate layout shown in FIG. 22 is developed to screen one TCR per plate from patient 8434. All the TMG/HLA combinations are pooled in one plate then one TCR is seeded in the plate. Every condition is in duplicate. HLA Clusters shown in FIG. 23 indicate the grouping of HLA plasmids for transfection into the COS-7 cells.

### 4.6.2 TCR Screening Results

As shown in the heatmap FIGS. 24B, 24C, & 24D, 3 TCRs are found to be reactive to the same combination of HLA group E and TMG1 in patient 8434. These 3 TCRs are clonotypes 20, 21 and 23 (8434-TCR20, 8434-TCR21 and 8434-TCR23). HLA cluster E contained DRA*01:01, DRB1*11:01, and DRB3*02:02.

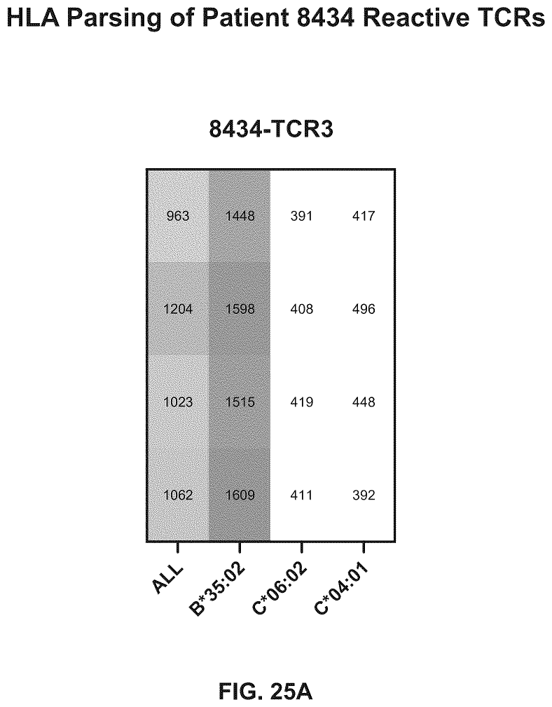

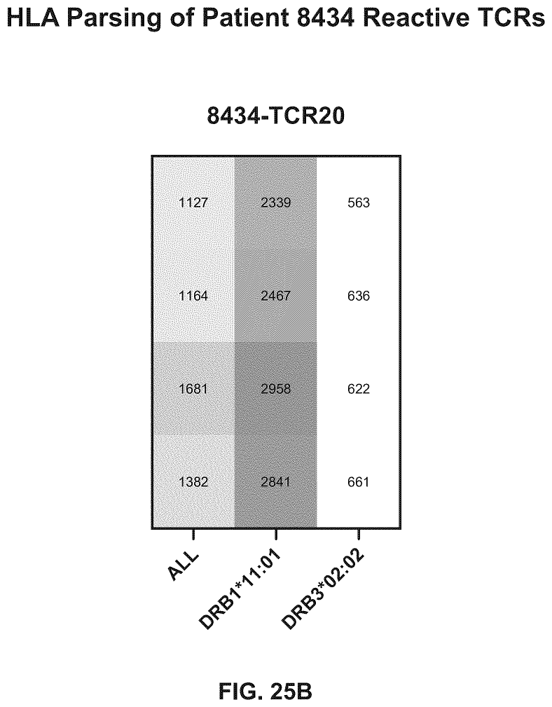

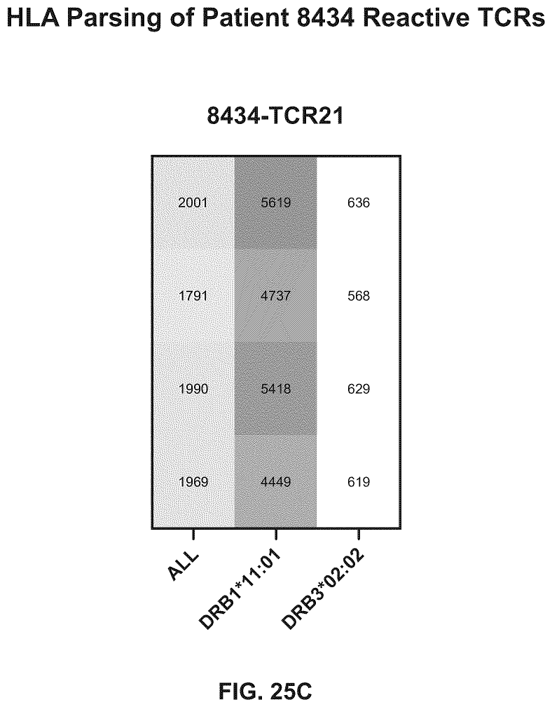

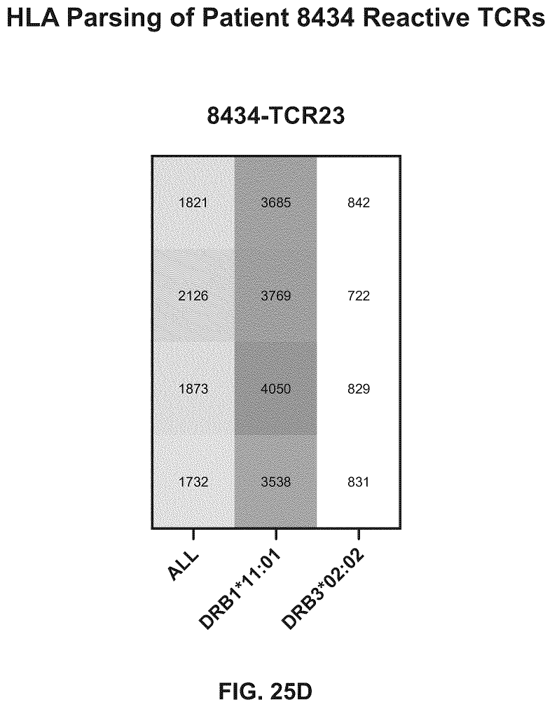

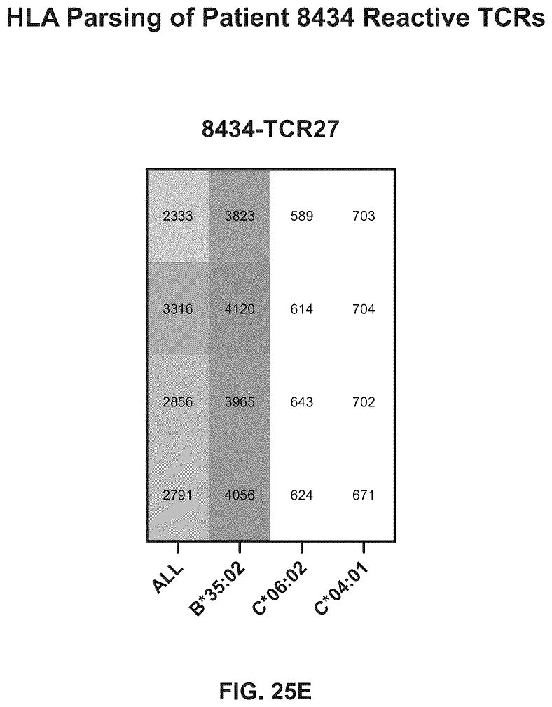

The further address the HLA allele specificity, COS-7 cells are transfected with individual HLA plasmids and TMG1 plasmid. TCRs 20, 21, and 23 are reactive to HLA DRB1*11:01 (FIGS. 25B, 25C, & 25D).

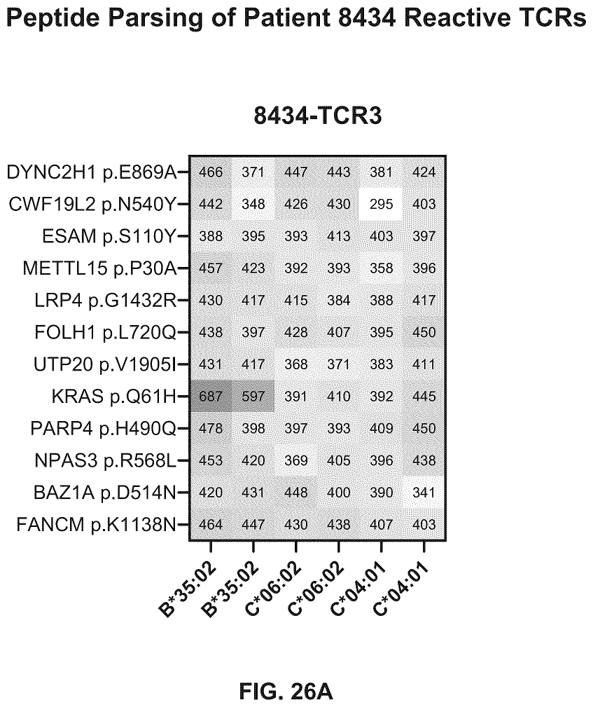

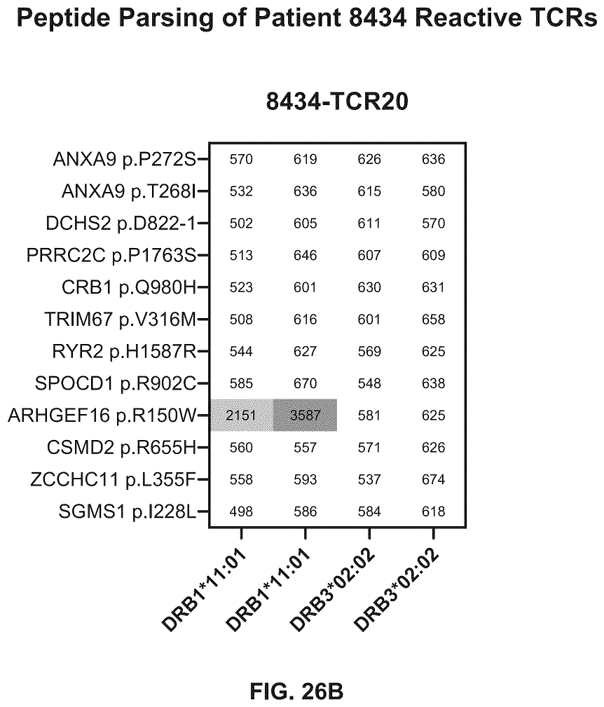

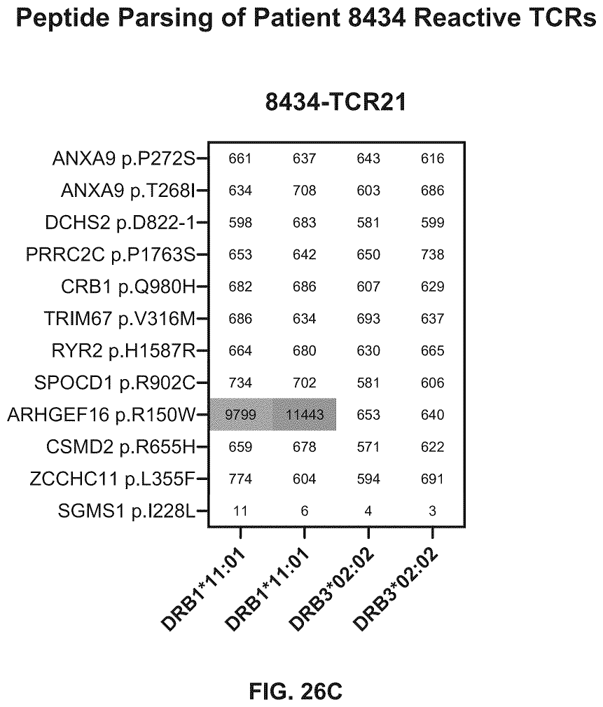

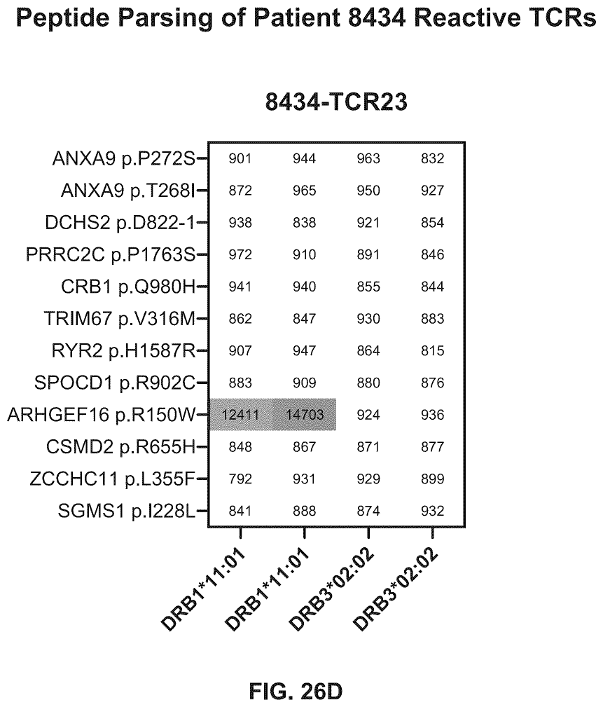

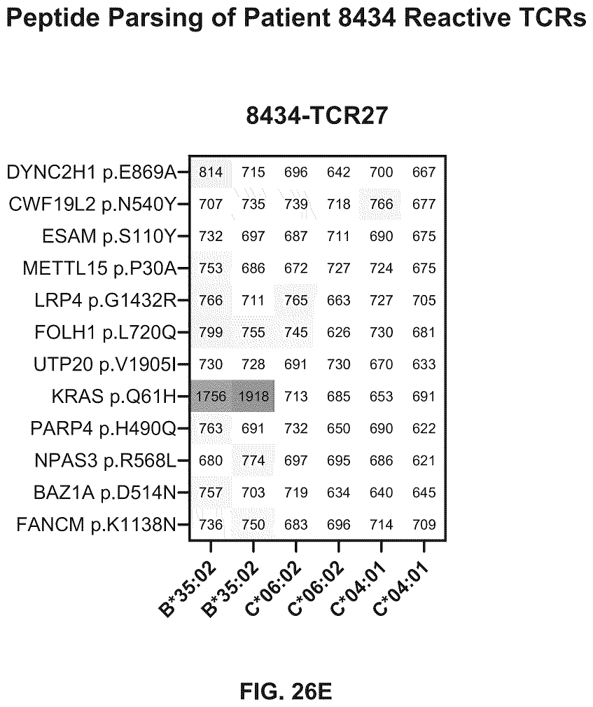

To further identify which neoantigen is involved in the TCR-neoantigen reactivity, each of the 12 peptides represented in TMG1 are pulsed, revealing that peptide 9 on TMG1 is the neo-reactive peptide for 8434-TCR20, 8434-TCR21 and 8434-TCR23 (FIGS. 26B, 26C, & 26D). Peptide 9 contains mutation ARHGEF16 p.R150W, which is found to have an allele frequency of 0.193 by WES, suggesting that it is a sub-clonal mutation in this patient's tumor.

In addition to the 3 TCRs identified against ARHGEF16 p.R150W, two TCRs (clonotypes 3 and 27, 8434-TCR3 and 8434-TCR27) are reactive to TMG2 with HLA cluster B (containing HLA B*35:02, C*06:02, and C*04:01) as indicated in FIGS. 24A & 24E.

To further resolve the HLA allele restriction, COS-7 cells are transfected with the individual HLA plasmids in cluster B and with TMG2 plasmid. This experiment reveals that HLA B*35:02 is the specific HLA restricting 8434-TCR3 and 8434-TCR27 (FIGS. 25A & 25E).

To further identify which neoantigen is involved in the TCR-neoantigen reactivity, each of the 12 peptides represented in TMG2 are pulsed, revealing that peptide 8 on TMG2 is the neo-reactive peptide for 8434-TCR3 and 8434-TCR27 (FIGS. 26A & 26E). Peptide 8 contains mutation KRAS p.Q61H which is found to have an allele frequency of 0.423 by WES, suggesting that it is a clonal mutation in this patient's tumor.

### 4.7 Conclusions

The series of data described in this example illustrate the application of a high-throughput TCR isolation and screening method in a patient derived tumor specimen wherein gene-expression data from sorted tumor infiltrating T cells are used to identify TCRs of interest for screening. Patient HLA and somatic tumor mutations are identified using NGS and bioinformatic analysis. Using a dissociated tumor sample from colorectal cancer Patient 8434, paired TCRα/β sequences are identified from tumor infiltrating T cells. Single-cell gene expression data is successfully used to cluster T cells into groups based on similar. Using the clustering, TCR sequences are identified for screening. The cluster analysis enable identification of rare TCR sequences of potential interest. These paired TCR sequences are reconstructed in silico from which DNA expression vectors encoding thirty-six TCRs from Patient 8434 are generated. Using the TCR screening method, all thirty-six TCRs are successfully screened and five TCRs, 8434-TCR3, 8434-TCR20, 8434-TCR21, 8434-TCR23, and 8434-TCR27, are found to recognize neoantigens from the patient's tumor. Three of the TCRs, 8434-TCR20, 8434-TCR21, and 8434-TCR23 are specific for the ARHGEF16 p.R150W neoantigen when presented in the context of HLA-DRB1*11:01. The other two TCRs, 8434-TCR3 and 8434-TCR27 recognize KRAS p.Q61H neoantigen in the context of HLA-B*35:02. KRAS p.Q61 is the third most frequently substituted amino acid residue in cancers and Histidine is the most common substituted amino acid at this position (COSMIC). KRAS p. Q61H is a common mutation in gastrointestinal cancers (e.g., colon and pancreatic cancers). Overall, these data demonstrate a process by which patient tumor mutations and HLA are used to screen TILs-derived TCR sequences obtained through single-cell gene-expression analysis. From these TCRs, neoantigen-specific TCRs are identified and functionally validated using a high-throughput TCR screening method. This method is used to identify potentially therapeutic TCRs. Importantly, these data demonstrate that this method can identify TCRs that recognize neoantigens that are common in many different cancers.

### Example 5: Patient 8434 TIL Screening

### 5.1 Patient/Sample Information

Patient 8434 demographic information is provided in Example 4.

### 5.2 Mutation and HLA Calling

Bioinformatic analysis to call Patient 8434 tumor's somatic mutations and HLA type was performed as described in Example 3. The somatic mutations and HLA type for patient 8434 are discussed in Example 4.

### 5.3 Expansion and Isolation of TILs

### 5.3.1 Culture Conditions and Methods

Patient 8434 dissociated tumor cells (DTCs) are stored in liquid nitrogen. DTCs are thawed in RPMI complete media (10% FBS, 1% Pen/Strip) and washed once. DTCs are counted with trypan blue using a hemocytometer. DTCs are cultured with irradiated PBMCs (three unrelated donors) using the Rapid Expansion Protocol (REP). Briefly, for the REP process, DTCs are plated with irradiated feeder cells at a ratio of 1:50 (DTCs:PBMCs) into a G-REX 100M culture vessel with IL-2 at 3000 IU/mL, 30 ng/mL OKT3 in 50:50 complete medium (50% RPMI 50% AIM-V supplemented with 5% human serum). Media is changed regularly during the REP. After 2 weeks, ex vivo expanded TILs are harvested and cryopreserved. 8434 TILs culture cell counts and viability are provided in Table 67.

TABLE 67
Patient 8434 TILs Culture Cell Counts and Viability

| Starting Cell Number | Total TILs Harvested | Viability at Harvest |
|---|---|---|
| 2.4e6 | 2.3e8 | 94.6% |

### 5.4 Ex Vivo Expanded TILs Screening

### 5.4.1 ELISpot Methods and Results

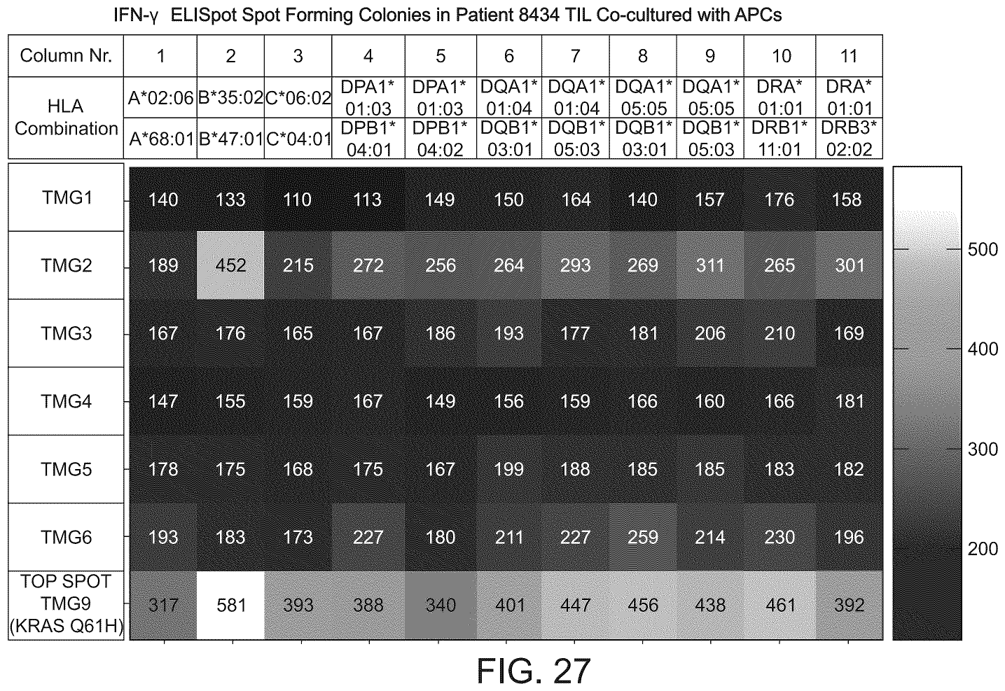

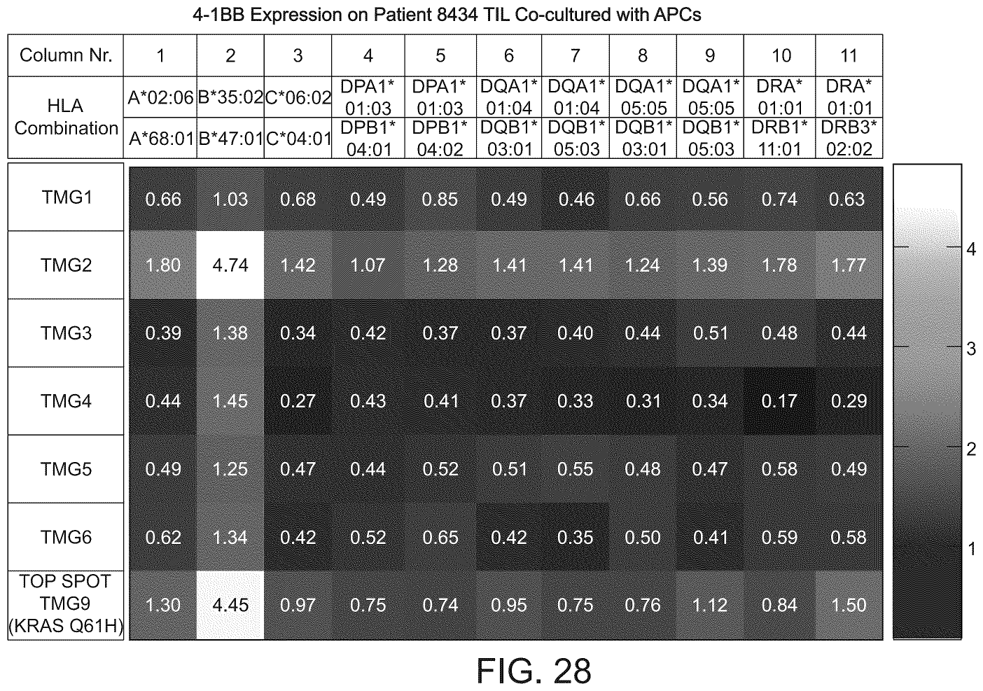

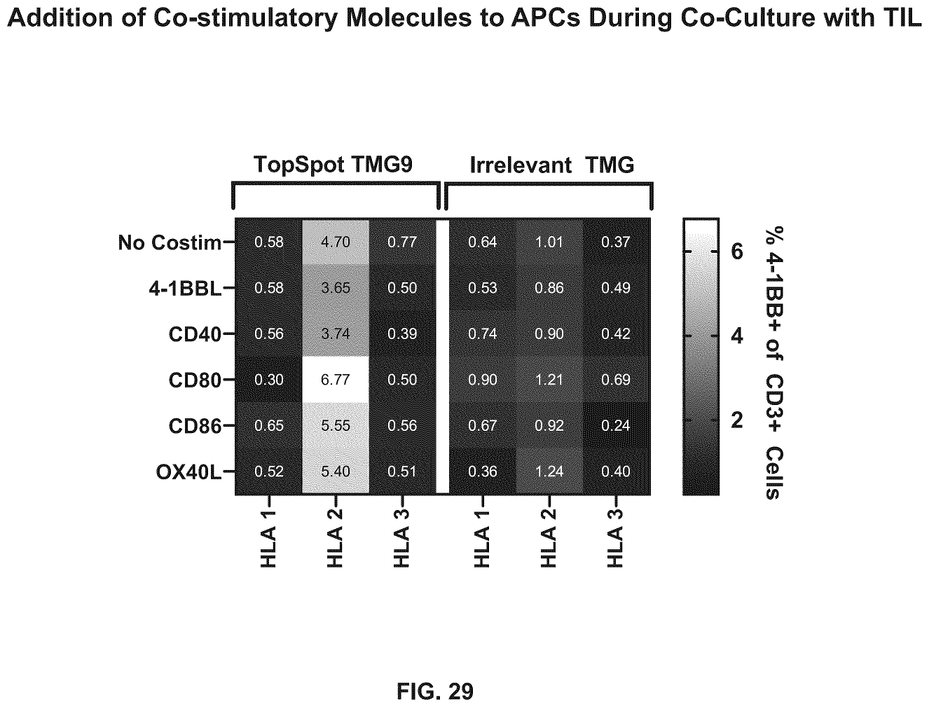

On day 1, COS-7 cells are seeded in 96 wells at 20,000 cells per well and incubated overnight at 37° C. TILs are thawed and recovered with IL-2 at 3000 IU/μL. On day 2, the cell medium is replaced with antibiotic-free DMEM medium prior to transfection. A total of 150 ng of TMG plasmid and 300 ng HLA plasmids are transfected using Lipofectamine 2000. To enhance screening sensitivity, 2 HLA plasmids (150 ng each) are transfected together in one well. Each condition included alleles from only one HLA locus, i.e., A, B, C, DP, DQ, or DR. Plasmids (450 ng total) and Lipofectamine (0.6 μL) are each diluted in 25 μL of OptiMEM medium. The DNA-containing tube (A) and lipofectamine-containing tubes (B) are each mixed well and incubated separately for 5 minutes at room temperature (RT). The contents of tube B are added to the contents of tube A, and the mixture is incubated for 20 minutes at room temperature to create the transfection mix. A total of 50 μL transfection mix is added to each well and cells are cultured overnight at 37° C. On day 3, the medium on the COS-7 cells is replaced with fresh medium containing peptide pools. Peptides are prepared at 50 mg/mL and pulsed at a final concentration of 10 μg/mL. Peptides are pooled together to mirror their grouping within each TMG. ELISpot plates are coated with anti-interferon gamma capture antibody (1-D1K) overnight. On day 4, ELISpot plates are washed with PBS and blocked with complete RPMI media (10% FBS) for 1 hour. COS-7 cells are harvested using trypsin. TILs are counted and resuspended at a concentration of 200k/mL. Medium is poured out from each ELISPOT plate. 50 μL of medium, 100 μL of COS-7 cells, and 100 μL of TILs (20,000 cells) are added sequentially to each well of the ELISpot plates. Plates are transferred to a 37° C. incubator with 5% CO₂ and incubated for 18-24 hours. On day 5, cells of each well in the ELISpot plate is mixed by pipetting, then 200 μL of cells are carefully transferred from the ELISpot plate to a new 96-well U-bottom plate. These cells are later stained using a cocktail of CD3, CD4, CD8, and 41BB antibodies for phenotyping with flow cytometry. ELISpot plates are washed and incubated with an anti-IFN-γ antibody (Biotinylated 7-B6-1 biotin). Plates are incubated at room temperature for 2 hours in the dark. Plates are washed and incubated with Streptavidin-ALP at room temperature for 1 hour in the dark. Plates are washed with PBS and stained with BCIP/NBT substrate solution. Plates are incubated at room temperature for 15 minutes until distinct spots appear. Tap water is used to wash the plates gently but extensively, then the plates are left out until completely dry. Plates are analyzed using an ELISpot reader. As shown in FIG. 27, wells containing TMG2 and top-spot TMG9 (both containing KRAS p.Q61H) had have higher signal compared to other TMGs. HLA group 2 including HLA B*35:02 and HLA B*47:01 had have the strongest signal in TMG2-containing wells and top-spot TMG9-containing wells. Additionally, TILs harvested from the co-culture plate are analyzed from 4-1BB expression by flow cytometry. Similar to the IFN-γ ELISpot findings, wells containing APCs transfected with TMG2 or top-spot TMG9 with HLA-B alleles exhibited increased 4-1BB expression within the CD3+ cell population (FIG. 28). HLA expression in COS-7 cells is verified with flow cytometry using an antibody cocktail against HLA-A2, HLA-DP, HLA-DQ and HLA-DR. Since COS-7 cells are not professional APCs, they lack co-stimulatory molecules that can enhance the activation of T cells with reactive TCRs. Therefore, in some co-culture conditions, the COS7 cells are co-transfected with HLA and TMG with or without select co-stimulatory molecules (4-1BBL, CD40, CD80, CD86, or OX40L). As shown in FIG. 29, evaluation of 4-1BB expression on T cells in these co-cultures revealed that additional of CD80, CD86, and OX40L, but not 4-1BBL or CD40 increased the measured 4-1BB upregulation in activating conditions (i.e., HLA Group 2+TopSpot TMG9) while having little to no effect in non-activating conditions (i.e., HLA Groups 1 or 2+TopSpot TMG9 or HLA Groups 1-3+Irrelevant TMG).

### 5.5 TILs Hunting

### 5.5.1 Co-Culture Experimental Design and Methods

Based on the ELISpot results above, TILs from patient 8434 are co-cultured with COS-7 cells transfected with TMG2 and HLA B plasmids, i.e., ‘STIM’ condition. COS-7 parental cells are incubated with TILs as a negative control, i.e., ‘no transfection control’ (NTC). Both conditions are incubated for 4 hours and overnight.

### 5.5.2 Cell Sorting and Preparation for 10× (Both 4 hr and ON)

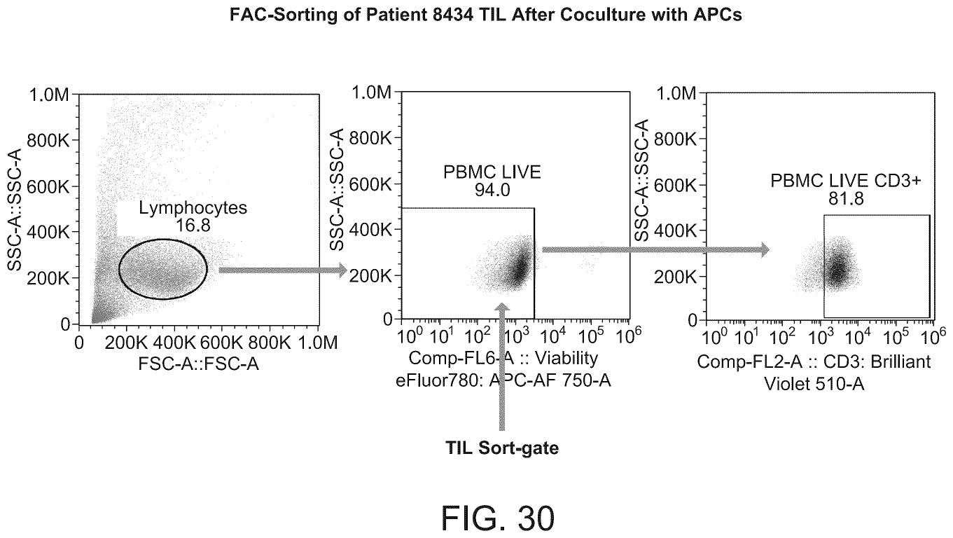

Cells are sorted using a SONY SH800 cell sorter using a viability dye and anti-CD3, anti-CD4, anti-CD8, and anti-41BB antibodies. Cells are sorted for lymphocyte and live cells as NEAT for both the 4 hour and overnight conditions. The gating schema and sort-gates for a representative expanded TILs sample from patient 8434 is shown in FIG. 30. Sufficient cells are recovered to target 10,000 cells in scRNAseq analysis. Viability is 99% for the 4-hour NTC and STIM conditions. Viability is 93% and 100% for the overnight NTC and STIM conditions, respectively.

### 5.5.3 Co-Culture Sorting

At 4 hours, 41BB is expressed at 4.07% in the STIM sample compared with 0.37% in the NTC samples on the CD3+CD8+ gate. In the overnight conditions, 41BB is expressed in 8.52% events in the STIM condition compared with 0.01% in the NTC condition. This suggested that a substantial number of cells are activated after culture with COS-7 cells in the STIM condition.

### 5.5.4 10× Chromium, Library Prep, and Sequencing

To retrieve full length TCR sequences, sorted TILs samples are prepared into paired end libraries and sequenced as described in Example 3. Each sequencing run yielded between 2×361.85 million and 2×551.15 million reads pass filter and between 93.18% and 96.69% of non-index bases achieved >=Q30 quality score.

5.5.5 scRNAseq Bioinformatics

### 5.5.5.1 Clustering and Mapping Reactive TCRs Back to Clusters

GEX reads are aligned to the human GRCh38 reference genome. Cell barcode assignment and unique molecular identifier (UMI) counts are then performed to create a single-cell gene expression matrix. Doublets and cells with >10% mitochondrial gene counts are filtered out. Raw read counts are normalized and scaled using Seurat. Approximately 2,000 highly variable genes are identified using the FindVariableGenes module. Principal component analysis (PCA) and uniform manifold approximation and projection (UMAP) are performed for dimension reduction and a shared nearest neighbor (SNN) algorithm is applied to cluster the cells.

5.5.5.2 Gene Expression and Signature within Reactive Clusters (4 hr, Overnight)

After the functional validation of neo-reactive TCRs, the barcodes of the cells containing these TCRs are projected onto the GEX data to map the gene expression profile of each neo-reactive TCR-containing cell. All cells within the GEX data are categorized into two groups: neo-reactive and non-neo-reactive. The function FindMarkers is performed to find the differentially expressed genes (DEGs) between the two groups. DEGs are defined with statistical cutoffs: average log 2 fold change of at least 1 and an adjusted p-value of less than 0.05. These DEGs are predicted to be a transcriptomic feature associated with the antigen-specific T cell response. Common genes appearing in the DEGs of all patients represent a gene signature that may predict neo-reactive T cells.

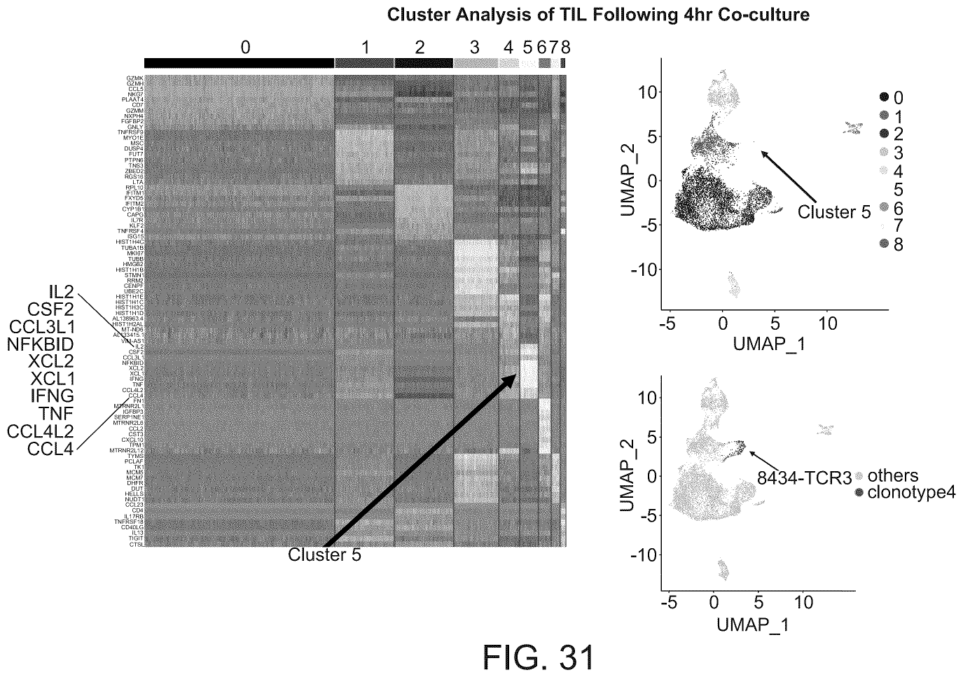

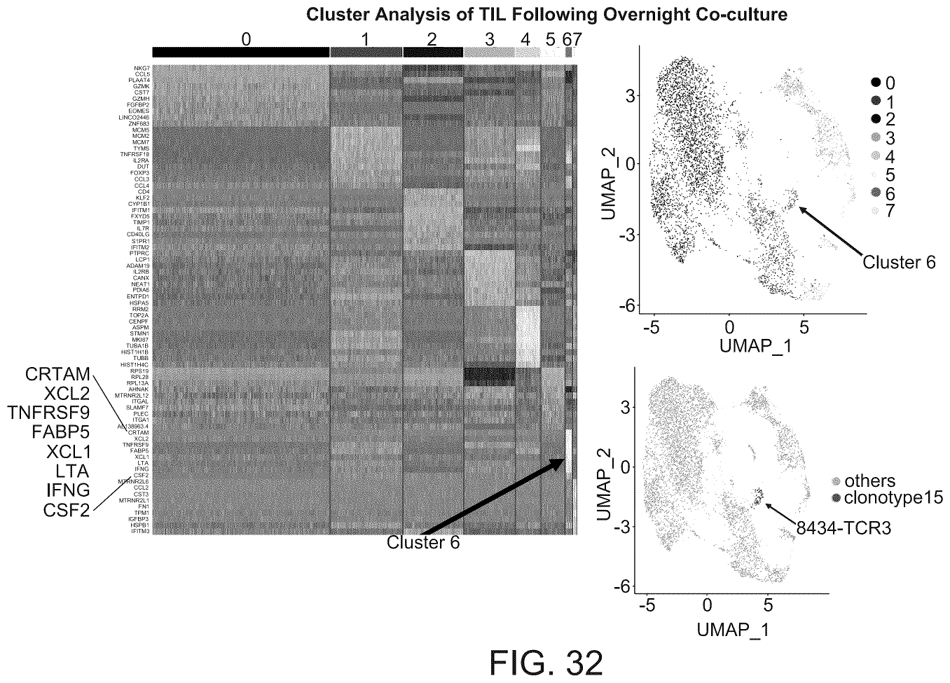

For both 4 hour and overnight co-cultures, the KRAS p.Q61H HLA-B*35:02 reactive 8434-TCR3 sequence is detectable in the VDJ sequencing data. An overlay of 8434-TCR3 T cells within the Clusters identified that this TCR clonotype is the primary clonotype present in Cluster 5 and Cluster 6 for 4 hour and overnight co-cultures, respectively (FIGS. 31 & 32).

In the 4 hour and overnight time points with antigen stimulation, 112 and 115 DEGs are identified using the above-described approach (FIGS. 31 & 32). Intersecting the two DEG sets lead to the following 67 genes: XCL2, XCL1, IL2, CSF2, IFNG, CCL4, CCL4L2, TNF, CCL3, RGCC, TNFSF9, DUSP2, NFKBID, MIR155HG, NR4A3, EVI2A, CRTAM, ZBED2, FABP5, PIM3, NR4A1, IL10, TNFSF14, NR4A2, LINC00892, ZFP36L1, GZMB, MYC, SPRY1, KDM6B, EGR2, PHLDA1, PPPIR2, VSIR, REL, PRDX1, SLA, CYTOR, DDX21, IER3, PGAM1, NAMPT, HSP90AB1, IL23A, FAM107B, BCL2A1, ZEB2, ZBTB32, BTG2, GADD45B, RILPL2, SEMA7A, TGIF1, SRGN, RAN, CFLAR, MAT2A, SIAH2, PRNP, RNF19A, FASLG, NME1, EVI2B, HSPH1, NOP16, CSRNP1, TAGAP.

### 5.6 Conclusions

The series of data described in this example illustrate a method by which high-throughput screening of TILs paired with single cell gene-expression and TCR sequencing can be utilized to identify and isolate neoantigen-specific TCRs. Using this method, reactive T cell clusters were successfully identified by gene-expression analysis. Neoantigen-specific TCR 8434-TCR3 are identified within the activated TILs clusters suggesting that this TILs screening method is an alternative or complimentary screening method to those described in Examples 3 and 4. Moreover, these data suggest that given a sufficiently oligoclonal TILs sample, neoantigen-reactive TILs can be readily identified in ex vivo expanded TILs from primary tumor samples. This method is utilized to identify therapeutically useful TCRs that could be applied to the treatment of cancers or other diseases.

### Example 6: Patient 6932 TCR Screening

### 6.1 Mutation and HLA Calling

### 6.1.1 Sample Demographics

Patient 6932 is a female, breast cancer (invasive/infiltrating ductal, Stage III-A) patient. Patient 6932's tumor specimen was collected from the left breast when the patient was 66 years old and prior to the start of treatment for the cancer diagnosis. A specimen of dissociated tumor cells (DTCs) from this patient is procured through a commercial vendor (Discovery Life Sciences; Huntsville, AL). A matched PBMC sample collected from the patient is used for the normal reference tissue.

### 6.1.2 Molecular Profiling Sample Processing

DNA and RNA are isolated from each of a dissociated tumor sample and from a matched PBMC sample. Quantification by fluorescence spectrometry indicated that yields are sufficient for downstream applications, and gel electrophoresis demonstrated an absence of degradation in the isolated genomic DNA. RNA is found to be of sufficient quality for paired end library preparation.

To assess somatic mutations, 200 ng tumor and 200 ng normal DNA are each processed through whole exome sequencing library preparation by way of hybrid capture. The final paired end libraries are sequenced on an Illumina NextSeqDx sequencer. Libraries are sequenced at 2×101 bp read lengths. The sequencing run yielded sufficient read number and quality for subsequent analysis. Reads are subject to on-board demultiplexing to yield paired FASTQ files.

To assess the gene expression of transcripts of interest, 50 ng of RNA isolated from the dissociated tumor sample is processed through RNAseq library preparation by way of hybrid capture. The final paired end library is sequenced on an Illumina NextSeqDx sequencer at 2×76 bp read lengths. The sequencing run yielded sufficient read number and quality for subsequent analysis. Reads are subject to on-board demultiplexing to yield paired FASTQ files.

### 6.1.3 Molecular Profiling Analysis Pipeline

Bioinformatic analysis to profile somatic mutations and HLA alleles for sample 6932 is performed as described in Example 3.

### 6.1.4 Molecular Profiling Results

WES analysis revealed 35 somatic non-synonymous mutations ranging in allele frequencies from 0.074 to 0.747 and gene expression values ranging from 0 to 111.883 FPKM. The somatic mutations produce 35 unique in silico neoantigen candidates.

### 6.2 Synthetic Mutation Assembly

### 6.2.1 Neoantigen Reagent Design

To create peptide fragments containing the patient's somatic mutations, a total of 74 in silico neoantigen candidates representing non-synonymous mutations are synthesized with crude quality. To create vectors containing mutations in nucleic acid form, amino acid sequences are reverse translated in silico and codon optimized for expression in human cells. A total of 3 TMGs are designed by concatenating a set of 11 sequences into one open reading frame as described in Example 3.

### 6.2.2 TMG Plasmid Synthesis

TMGs are synthesized as described in Example 3.

### 6.3 Human Leukocyte Antigen (HLA) Plasmid Assembly

Plasmids encoding the patient's HLA alleles are designed and synthesized as described in Example 3.

6.4 Single-cell Analysis of TILs Sorted from Dissociated Tumor Sample

### 6.4.1 Cell Sorting and Preparation

Cells are washed as previously described in Example 3. A total of 10% of cells are set aside to grow TILs. The remaining cells are stained with anti-CD3 and anti-CD45 antibodies. CD3+CD45+ cells are sorted with a SONY SH800 cell sorter. Cells are subsequently washed with BSA 0.2% and resuspended in an appropriate volume of BSA 0.2%. Sorted cells are 97% viable and prepared at a concentration of 480 cells/μL, enabling targeting of 10,000 cells in subsequent processing.

### 6.4.2 Single-Cell Paired End Library Preparation and Sequencing

The sorted tumor sample is processed to create paired end libraries and sequenced as described in Example 3. The sequencing run yielded sufficient read number and quality for subsequent analysis.

6.4.3 scRNAseq Analysis

The GEX and VDJ sequencing data are preprocessed using the CellRanger toolkit (version 5.1) provided by 10× Genomics. The BCL files were converted to raw FASTQ files. The FASTQ files for the GEX and VDJ experiments are processed separately.

GEX reads are aligned to the human GRCh38 reference genome. Cell barcode assignment and unique molecular identifier (UMI) counts are then performed to create a single-cell gene expression matrix. Doublets and cells with >10% mitochondrial gene counts are filtered out. Raw read counts are normalized and scaled using Seurat. Approximately 2,000 highly variable genes are identified using the FindVariableGenes module. Principal component analysis (PCA) and uniform manifold approximation and projection (UMAP) are performed for dimension reduction and a shared nearest neighbor (SNN) algorithm is applied to cluster the cells.

The raw V(D)J sequencing reads are assembled into contigs using a graph-based algorithm with the aid of the pre-built reference sequence from the IMGT (www.imgt.org) database. Cells with identical productive V(D)J transcripts are considered to belong to the same clonotype. The following are reported for each unique clonotype: the amino acid sequence of the CDR3 region, the full-length FASTA sequence of the TRA chain, the full-length FASTA sequence of the TRB chain, and the clonotype frequency, defined as the number of cells in which each clonotype is observed.

The number of detected cells, mean reads per cell, reads mapped to the genome etc. were reported by CellRanger as quality control measurements.

To ensure that every T cell in the study has both gene expression information and its TCR sequences, only cells detected in both GEX and V(D)J are used in the analysis.

### 6.5 TCR Reconstruction

### 6.5.1 T-Cell Receptor Assembly

Single-cell RNAseq analysis yielded 547 clonotypes where 413 clonotypes contain exactly one beta chain and one alpha chain. Clonotype frequency ranges from 1 to 45 cells with 119 clonotypes observed in more than one cell and 12 clonotypes observed in 10 or more cells. From the 20 most frequent clonotypes, 22 alpha and 18 beta chains are modified and assembled to create 22 individual TCRs. Alpha and beta chains are modified as described in Example 3.

### 6.5.2 TCR Plasmid Assembly

Alpha and beta chains are synthesized and cloned into TCR plasmids as described in Example 3.

### 6.6 In Vitro TCR Screening

### 6.6.1 Experimental Design and Methods

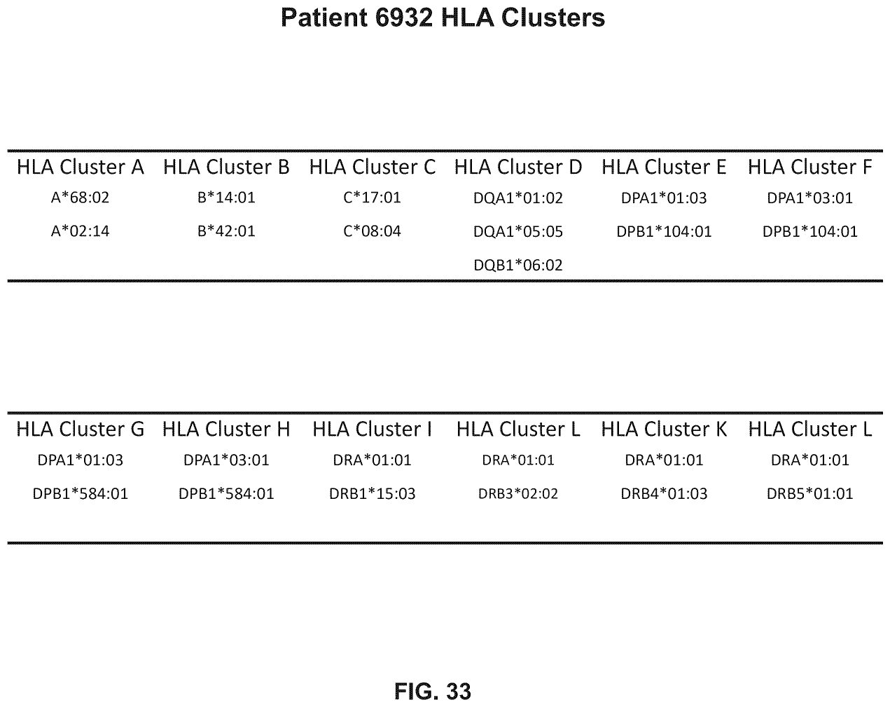

On day 1, COS-7 cells are seeded in 96 wells at 20,000 cells per well and incubated overnight at 37° C. On day 2, each well of COS-7 cells is transfected with 150 ng of TMG plasmids and 300 ng of HLA plasmids. On day 3, five million cells are electroporated with each of the 18 TCR plasmid and a negative control (NTC). On day 4, Jurkat cells are harvested and seeded on top of transfected COS-7 cells. After 5 hours of co-culture, cells are harvested, and luciferase activity is measured. There are 3 TMGs designed for the relevant mutations, and 22 TCRs are picked from single cell sequencing for patient 6932. In addition, this patient has 2 HLA-A, 2 HLA-B, 2 HLA-C, 2 HLA-DQ-A, 1 HLA-DQ-B, 2 DP-A, 2 DP-B, 1 DRB1, 1 DRB3, 1 DRB4, and 1 DRB5 alleles. The HLA plasmids are segregated into 12 groups (FIG. 33) to reduce the number of combinations with TMG plasmids. On the day of electroporation, H57 antibody is coated on the 96 multiwell plate overnight at 4° C. as a positive control. On the next day, Jurkat cells are seeded on the plate for 5 hours. Luciferase activity fold change (FC) is calculated based on cells seeded without H57 coating. All cells with electroporated TCRs exhibited higher luciferase activity in H57 coated condition suggesting that the TCRs are biologically functional.

All the TMG/HLA combinations are pooled in one plate then one TCR is seeded in the plate. Every condition is in duplicate allowing to confidently call the positive combinations. HLA Clusters shown in FIG. 33 indicate the grouping of HLA plasmids for transfection into the COS-7 cells.

### 6.6.2 TCR Screening Results

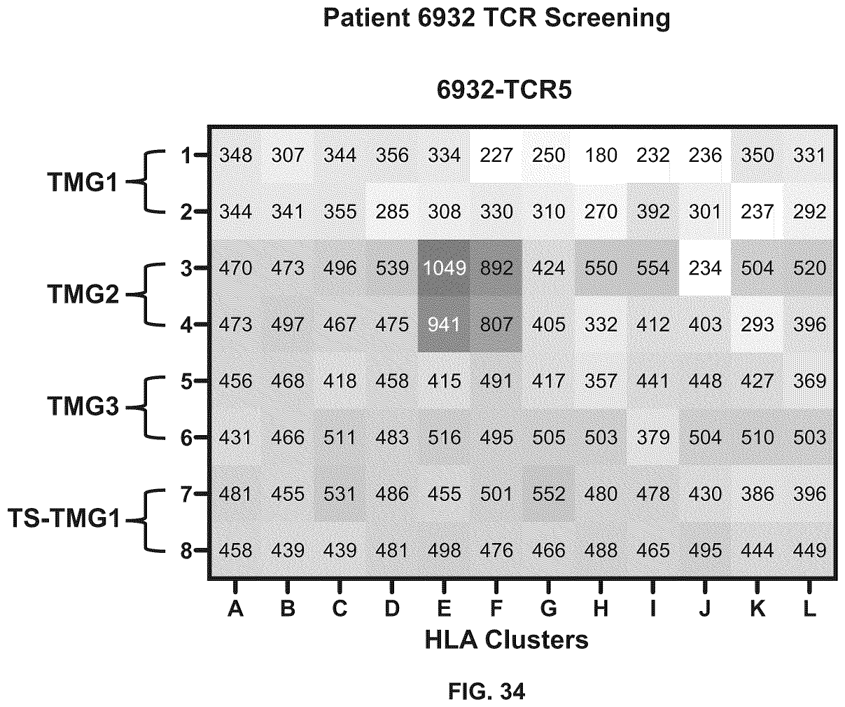

As shown in the heatmap FIGS. 34, 1 TCR was found to be reactive to the same combination of HLA group E and F and TMG2 in patient 6932. This reactive TCR is clonotype 5 (6932-TCR5). HLA cluster E contained DPA1*01:03 and DPB1*104:01. HLA cluster F contained DPA1*03:01 and DPB1*104:01.

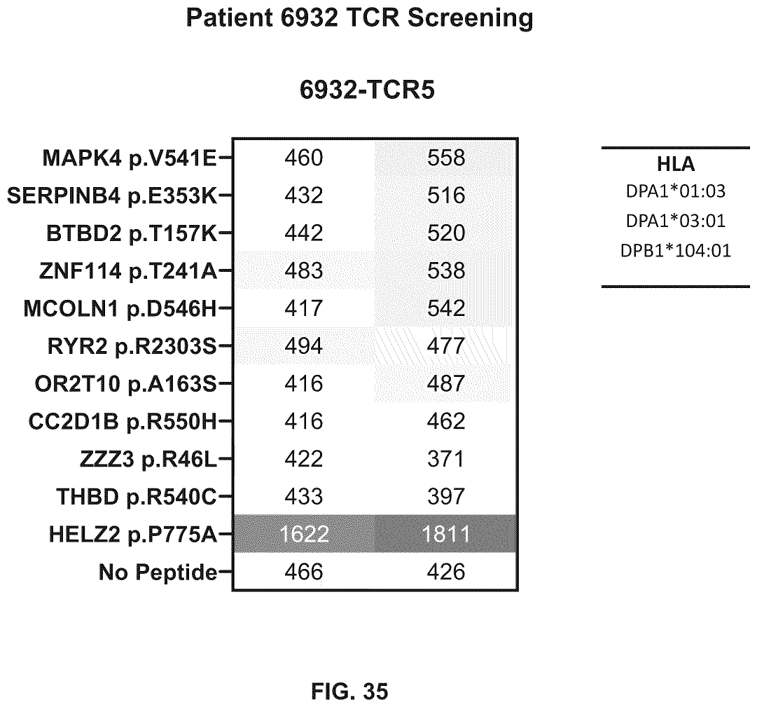

To further identify which neoantigen is involved in the TCR-neoantigen reactivity, each of the 11 peptides represented in TMG2 are pulsed, revealing that peptide 11 on TMG2 is the neo-reactive peptide for 6932-TCR5 (FIG. 35). Peptide 11 contains mutation HELZ2 p.P775A.

### 6.7 Conclusions

The series of data described in this example illustrate the application of a high-throughput TCR isolation and screening method in a patient-derived tumor specimen wherein gene-expression data from sorted tumor infiltrating T cells are used to identify TCRs of interest for screening. Patient HLA and somatic tumor mutations are identified using NGS and bioinformatic analysis. Using a dissociated tumor sample from breast cancer Patient 6932, paired TCRα/β sequences are identified from tumor infiltrating T cells. Single-cell gene expression data is successfully used to cluster T cells into groups based on similar transcriptional profiles. Using the clustering, TCR sequences are identified for screening. Cluster analysis enables the identification of rare TCR sequences of potential interest. These paired TCR sequences are reconstructed in silico from which DNA expression vectors encoding twenty-two TCRs from Patient 6932 are generated. Using the TCR screening method, all twenty-two TCRs are successfully screened and one TCR, 6932-TCR5 is found to recognize neoantigens from the patient's tumor. The reactive TCR, 6932-TCR5 is specific for HELZ2 p.P775A neoantigen when presented in the context of either HLA alleles DPA1*01:03 and DPB1*104:01 or DPA1*03:01 and DPB1*104:01. Overall, these data demonstrate a method for the high-throughput screening of TCRs identified from primary tumor samples. Furthermore, this example describes the identification of neoantigen reactive TCR 6932-TCR5.

### Example 7: Patient 0025 TCR Screening

### 7.1 Mutation and HLA Calling

### 7.1.1 Sample Demographics

Patient 0025 is a female, endometrial adenocarcinoma (Stage III-C) patient.

Patient 0025's tumor specimen was collected from the endometrium when the patient was 57 years old and prior to the start of treatment for the cancer diagnosis. A specimen of dissociated tumor cells (DTCs) from this patient is procured through a commercial vendor (Discovery Life Sciences; Huntsville, AL). A matched PBMC sample collected from the patient is used for the normal reference tissue.

### 7.1.2 Molecular Profiling Sample Processing

DNA and RNA are isolated from each of a dissociated tumor sample and from a matched PBMC sample. Quantification by fluorescence spectrometry indicated that yields are sufficient for downstream applications, and gel electrophoresis demonstrated an absence of degradation in the isolated genomic DNA. RNA is found to be of sufficient quality for paired end library preparation.

To assess somatic mutations, 200 ng tumor and 200 ng normal DNA are each processed through whole exome sequencing library preparation by way of hybrid capture. The final paired end libraries are sequenced on an Illumina NextSeqDx sequencer. Libraries are sequenced at 2×101 bp read lengths. The sequencing run yielded sufficient read number and quality for subsequent analysis. Reads are subject to on-board demultiplexing to yield paired FASTQ files.

To assess the gene expression of transcripts of interest, 50 ng of RNA isolated from the dissociated tumor sample is processed through RNAseq library preparation by way of hybrid capture. The final paired end library is sequenced on an Illumina NextSeqDx sequencer at 2×76 bp read lengths. The sequencing run yielded sufficient read number and quality for subsequent analysis. Reads are subject to on-board demultiplexing to yield paired FASTQ files.

### 7.1.3 Molecular Profiling Analysis Pipeline

Bioinformatic analysis to profile somatic mutations and HLA alleles for sample 0025 is performed as described in Example 3.

### 7.1.4 Molecular Profiling Results

WES analysis revealed 36 somatic non-synonymous mutations ranging in allele frequencies from 0.110 to 0.461 and gene expression values ranging from 0.02 to 2295.86 FPKM. The somatic mutations produce 36 unique in silico neoantigen candidates.

### 7.2 Synthetic Mutation Assembly

### 7.2.1 Neoantigen Reagent Design

To create peptide fragments containing the patient's somatic mutations, a total of 74 in silico neoantigen candidates representing non-synonymous mutations are synthesized with crude quality. To create vectors containing mutations in nucleic acid form, the amino acid sequences are reverse translated in silico and codon optimized for expression in human cells. A total of 3 TMGs are designed by concatenating a set of 12 sequences into one open reading frame as described in Example 3.

### 7.2.2 TMG Plasmid Synthesis

TMGs are synthesized as described in Example 3.

### 7.3 Human Leukocyte Antigen (HLA) Plasmid Assembly

Plasmids encoding the patient's HLA alleles are designed and synthesized as described in Example 3.

7.4 Single-cell Analysis of TILs Sorted from Dissociated Tumor Sample

### 7.4.1 Cell Sorting and Preparation

Cells are washed as previously described in Example 3. A total of 10% of cells are set aside to grow TILs. The remaining cells are stained with anti-CD3 and anti-CD45 antibodies. CD3+CD45+ cells are sorted with a SONY SH800 cell sorter. Cells are subsequently washed with BSA 0.2% and resuspended in an appropriate volume of BSA 0.2%. Sorted cells are 70% viable and prepared at a concentration of 100 cells/μL, enabling targeting of 2,000 cells in subsequent processing.

### 7.4.2 Single-cell Paired End Library Preparation and Sequencing

The sorted tumor sample is processed to create paired end libraries and sequenced as described in Example 3. The sequencing run yielded sufficient read number and quality for subsequent analysis.

7.4.3 scRNAseq Analysis

The GEX and VDJ sequencing data are preprocessed using the CellRanger toolkit (version 5.1) provided by 10× Genomics. The BCL files were converted to raw FASTQ files. The FASTQ files for the GEX and VDJ experiments are processed separately.

GEX reads are aligned to the human GRCh38 reference genome. Cell barcode assignment and unique molecular identifier (UMI) counts are then performed to create a single-cell gene expression matrix. Doublets and cells with >10% mitochondrial gene counts are filtered out. Raw read counts are normalized and scaled using Seurat. Approximately 2,000 highly variable genes are identified using the FindVariableGenes module. Principal component analysis (PCA) and uniform manifold approximation and projection (UMAP) are performed for dimension reduction and a shared nearest neighbor (SNN) algorithm is applied to cluster the cells.

The raw V(D)J sequencing reads are assembled into contigs using a graph-based algorithm with the aid of the pre-built reference sequence from the IMGT (www.imgt.org) database. Cells with identical productive V(D)J transcripts are considered to belong to the same clonotype. The following are reported for each unique clonotype: the amino acid sequence of the CDR3 region, the full-length FASTA sequence of the TRA chain, the full-length FASTA sequence of the TRB chain, and the clonotype frequency, defined as the number of cells in which each clonotype is observed.

The number of detected cells, mean reads per cell, reads mapped to the genome etc. were reported by CellRanger as quality control measurements.

To ensure that every T cell in the study has both gene expression information and its TCR sequences, only cells detected in both GEX and V(D)J are used in the analysis.

### 7.5 TCR Reconstruction

### 7.5.1 T-Cell Receptor Assembly

Single-cell RNAseq analysis yielded 3414 clonotypes where 2192 clonotypes contain exactly one beta chain and one alpha chain. Clonotype frequency ranges from 1 to 339 cells with 766 clonotypes observed in more than one cell and 74 clonotypes observed in 10 or more cells. From the most frequent 110 clonotypes, 59 and 55 alpha and beta chains, respectively, are modified and assembled to create 61 individual TCRs. Alpha and beta chains are modified as described in Example 3.

### 7.5.2 TCR Plasmid Assembly

Alpha and beta chains are synthesized and cloned into TCR plasmids as described in Example 3.

### 7.6 In Vitro TCR Screening

### 7.6.1 Experimental Design and Methods

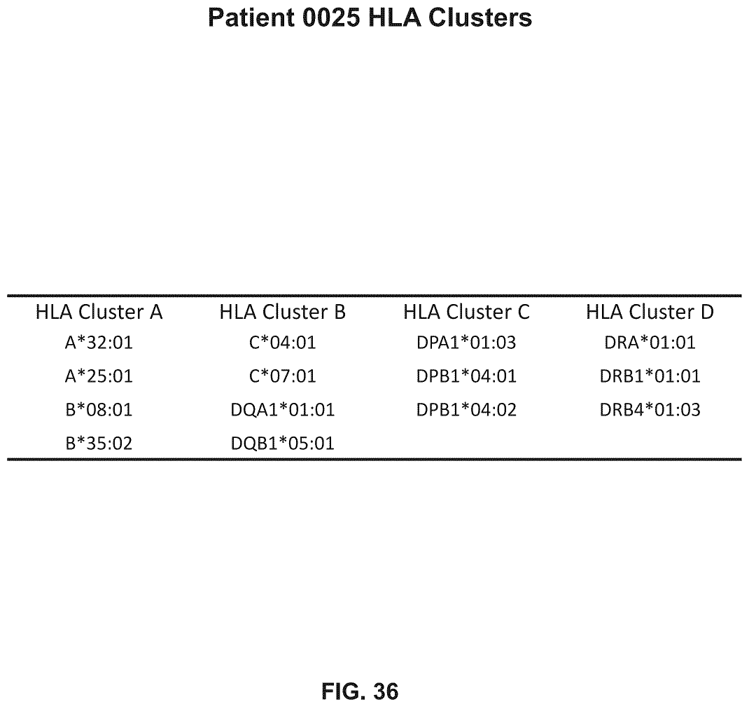

On day 1, COS-7 cells are seeded in 96 wells at 20,000 cells per well and incubated overnight at 37° C. On day 2, each well of COS-7 cells is transfected with 150 ng of TMG plasmids and 300 ng of HLA plasmids. On day 3, five million cells are electroporated with each of the 18 TCR plasmid and a negative control (NTC). On day 4, Jurkat cells are harvested and seeded on top of transfected COS-7 cells. After 5 hours of co-culture, cells are harvested, and luciferase activity is measured. There are 3 TMGs designed for the relevant mutations, and 61 TCRs are picked from single cell sequencing for patient 0025. In addition, this patient has 2 HLA-A, 2 HLA-B, 2 HLA-C, 1 HLA-DQ-A, 1 HLA-DQ-B, 1 DP-A, 2 DP-B, 1 DRB1, and 1 DRB4 allele. The HLA plasmids are segregated into 4 groups (FIG. 36) to reduce the number of combinations with TMG plasmids. On the day of electroporation, H57 antibody is coated on the 96 multiwell plate overnight at 4° C. as a positive control. On the next day, Jurkat cells are seeded on the plate for 5 hours. Luciferase activity fold change (FC) is calculated based on cells seeded without H57 coating. All cells with electroporated TCRs exhibited higher luciferase activity in H57 coated condition suggesting that the TCRs are biologically functional.

All the TMG/HLA combinations are pooled in one plate then one TCR is seeded in the plate. Every condition is in duplicate allowing to confidently call the positive combinations. HLA Clusters shown in FIG. 36 indicate the grouping of HLA plasmids for transfection into the COS-7 cells.

### 7.6.2 TCR Screening Results

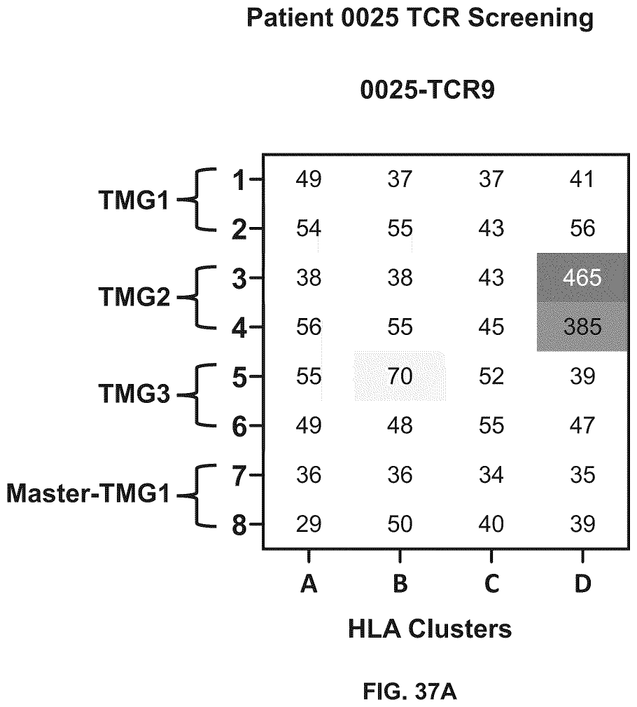

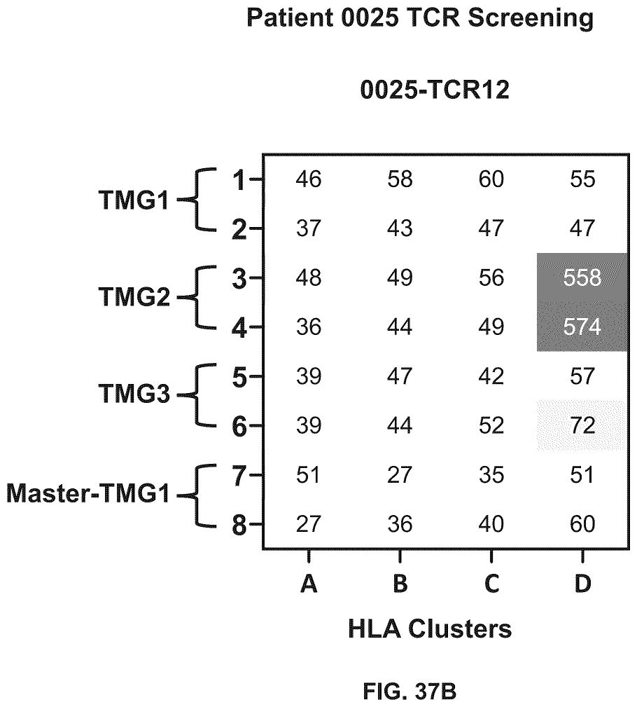

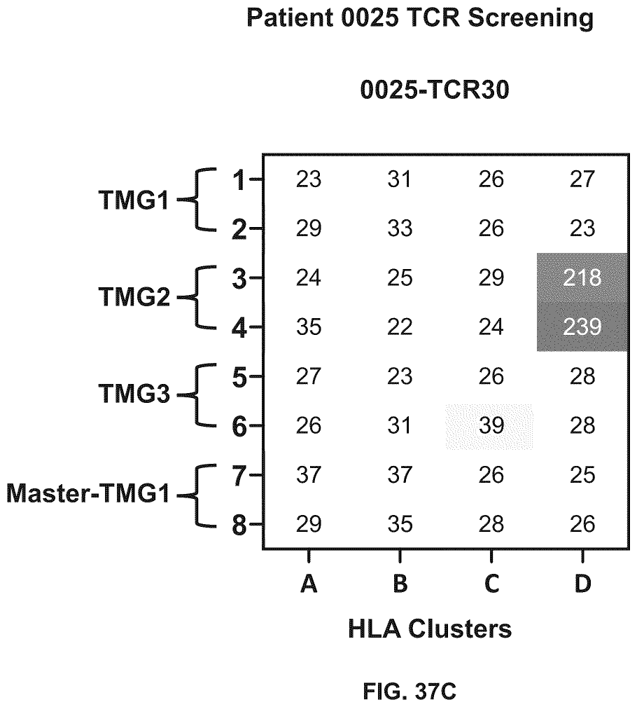

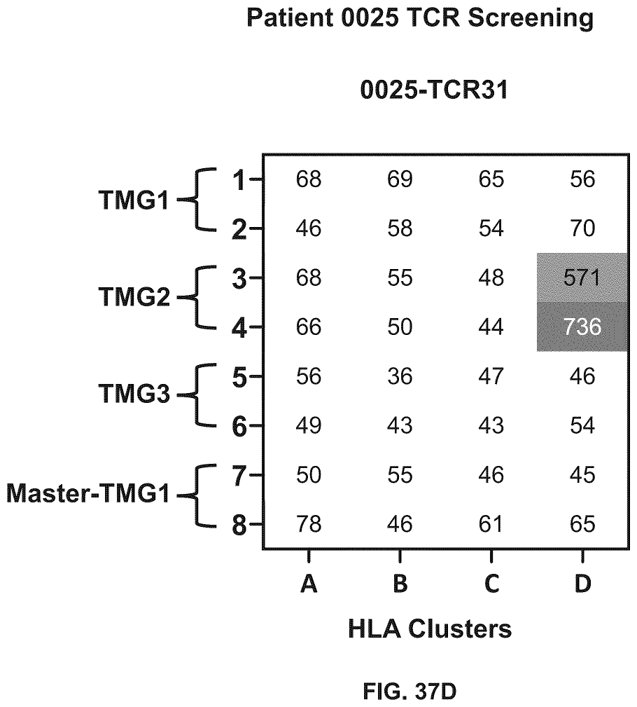

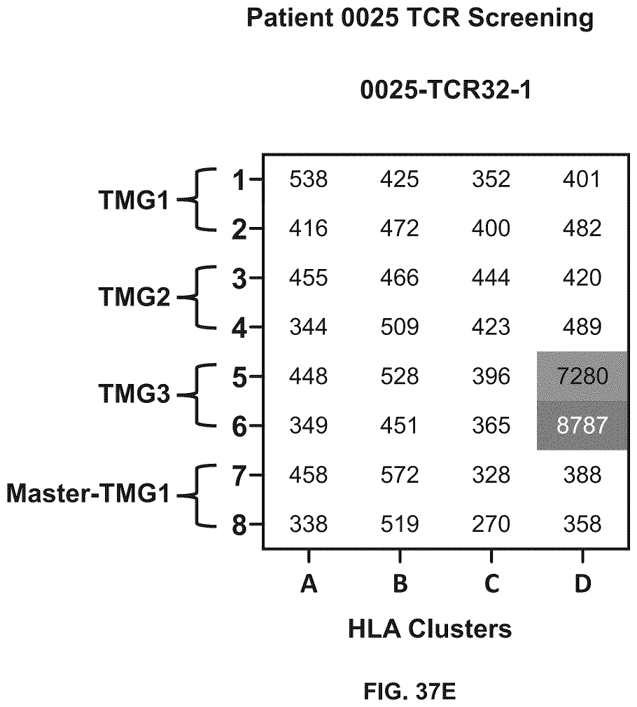

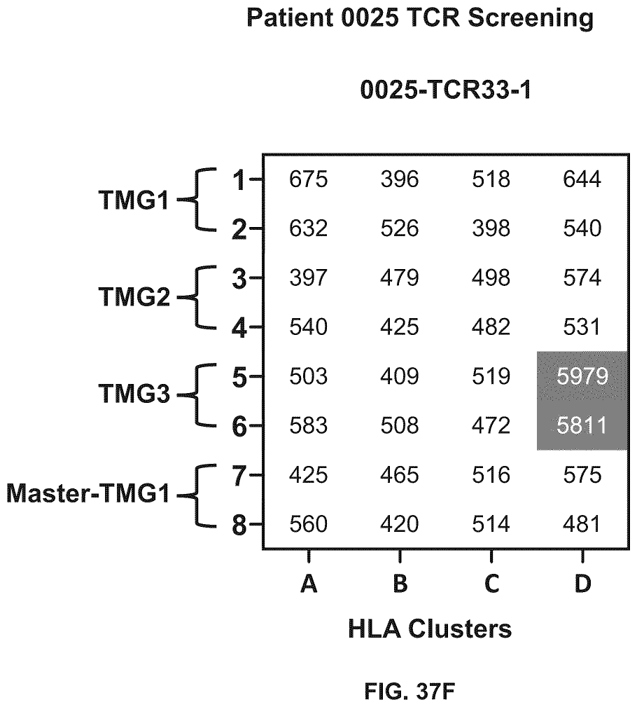

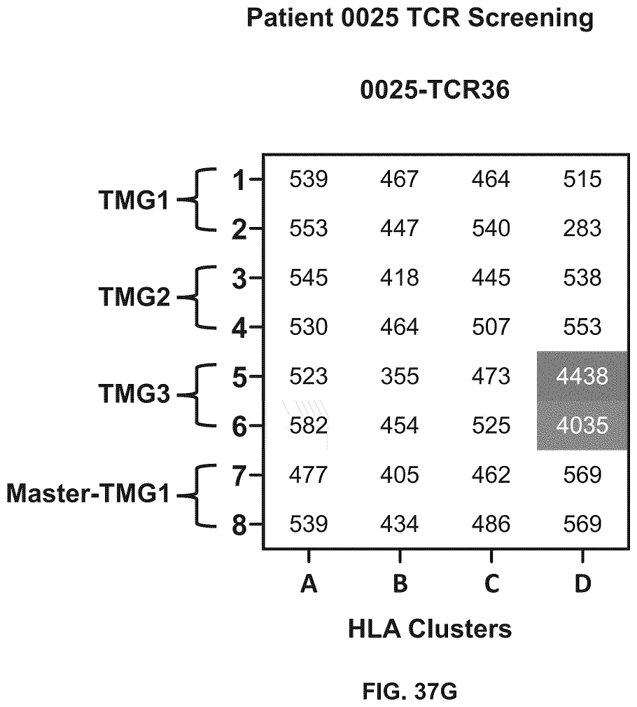

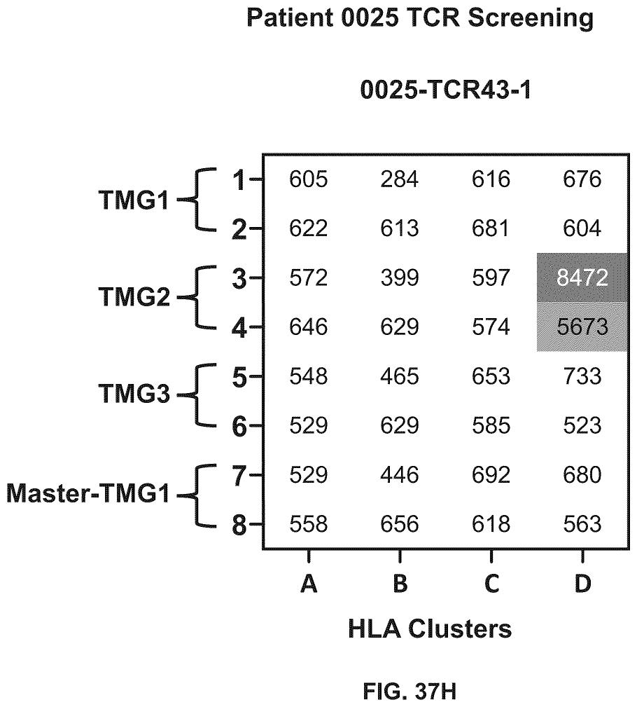

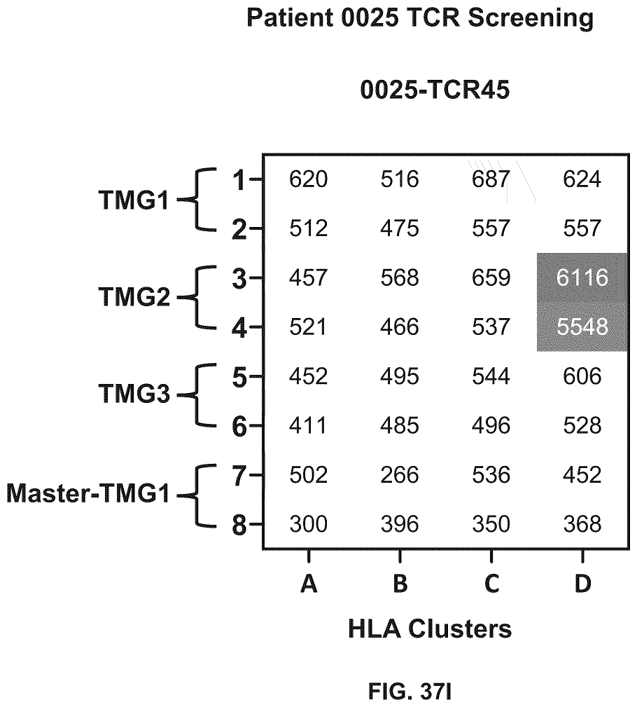

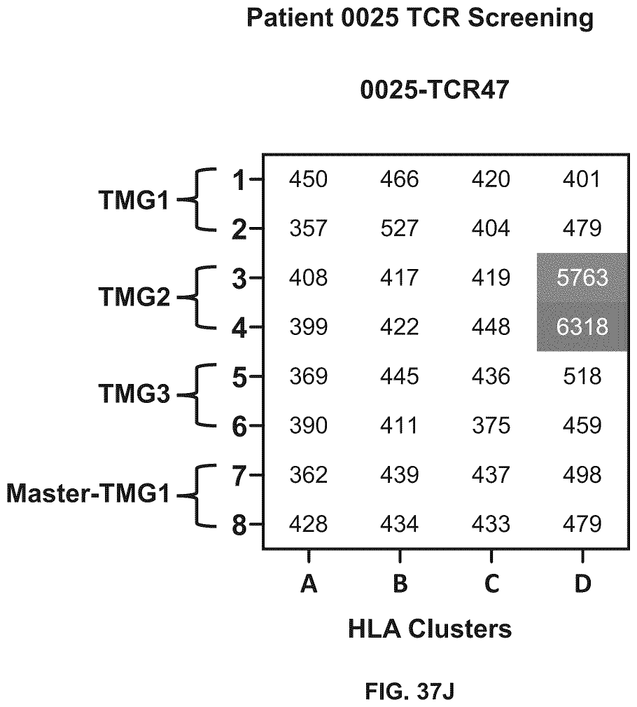

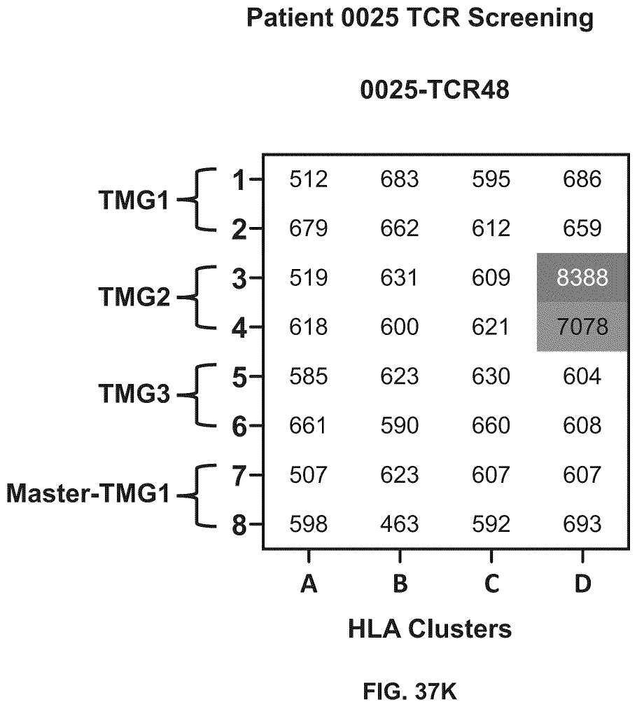

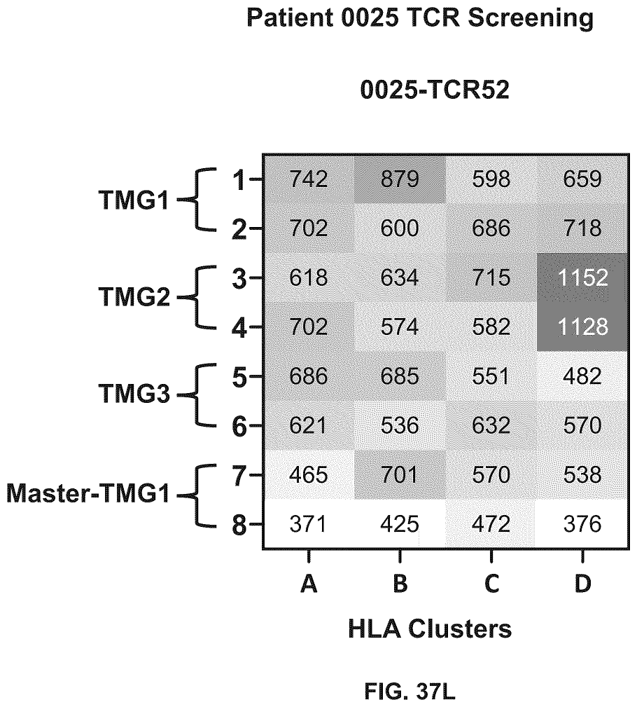

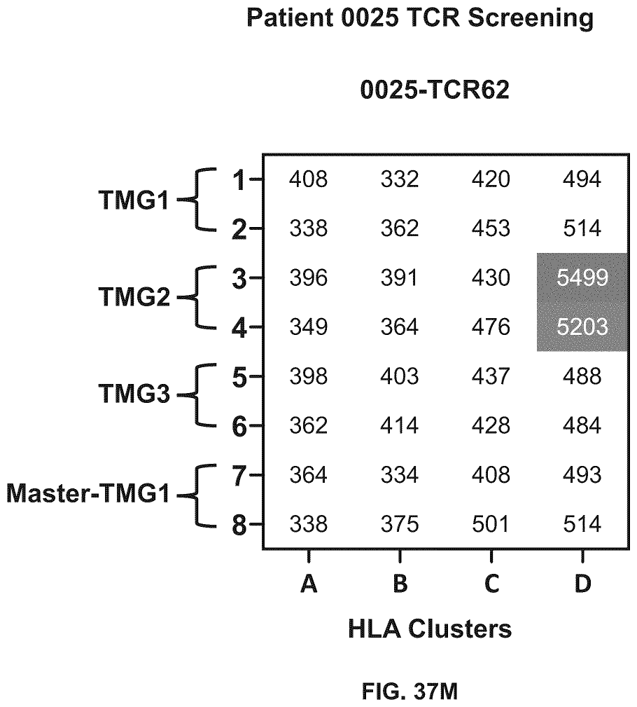

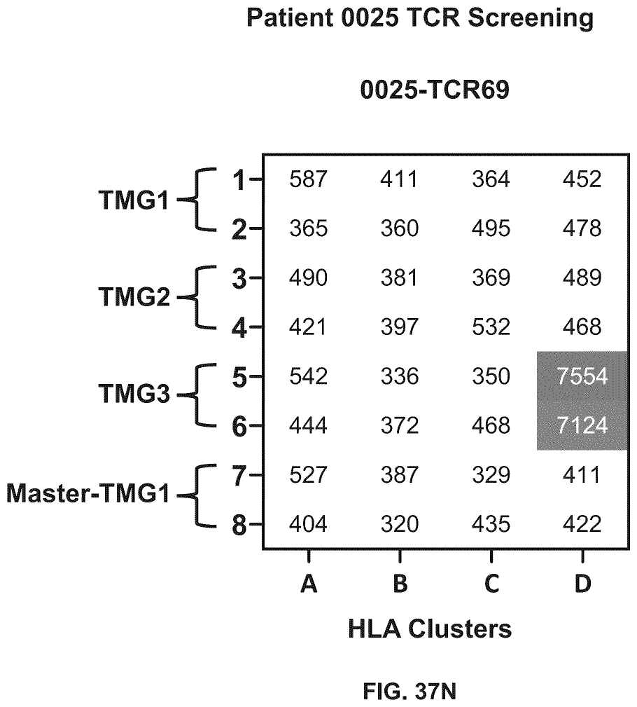

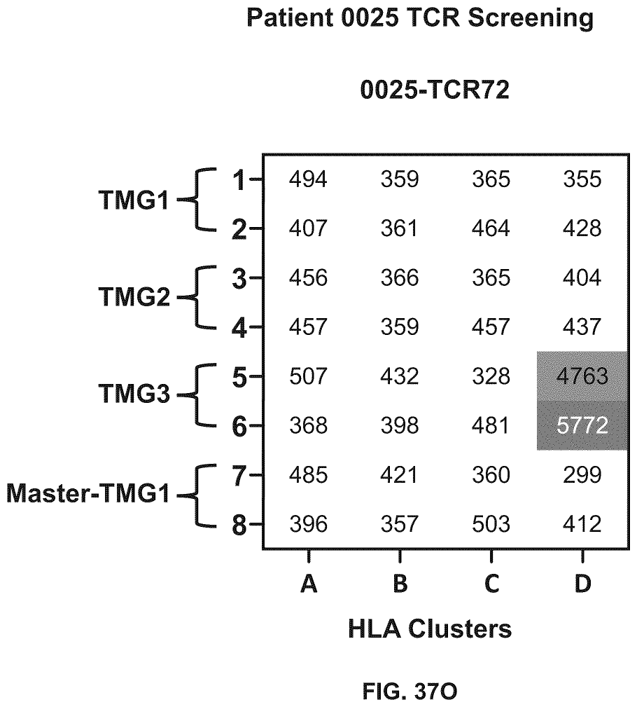

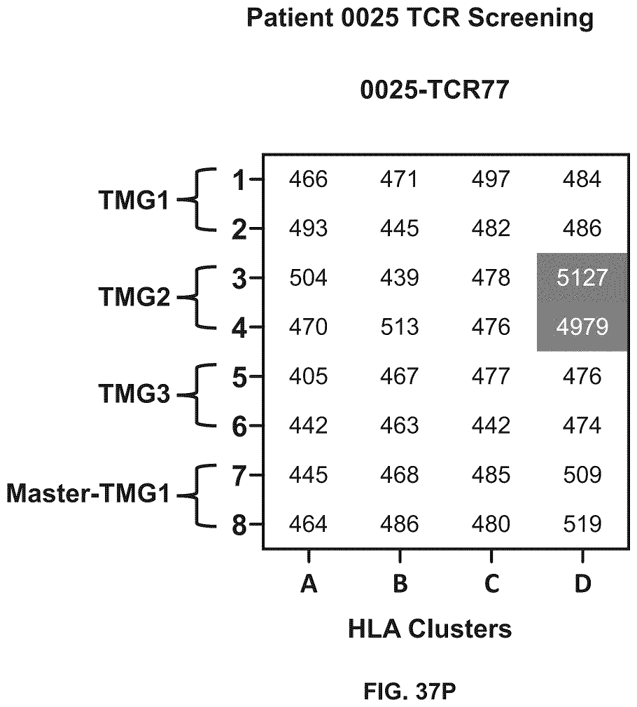

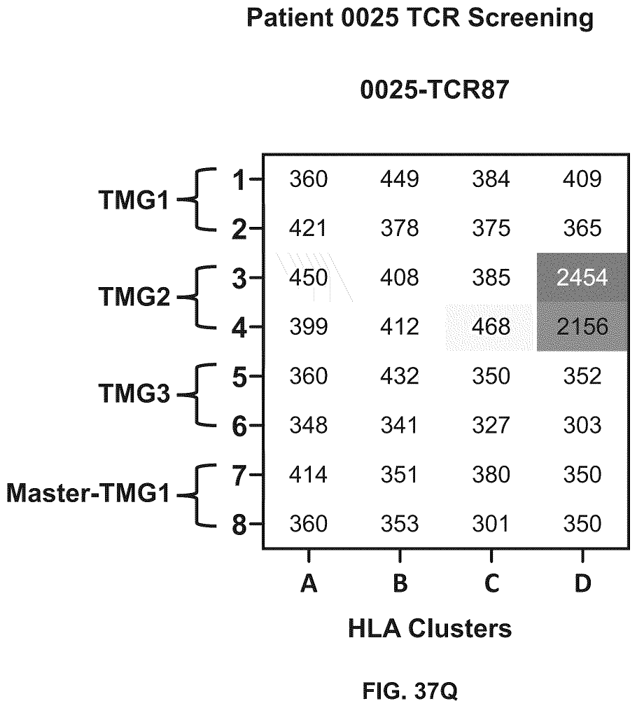

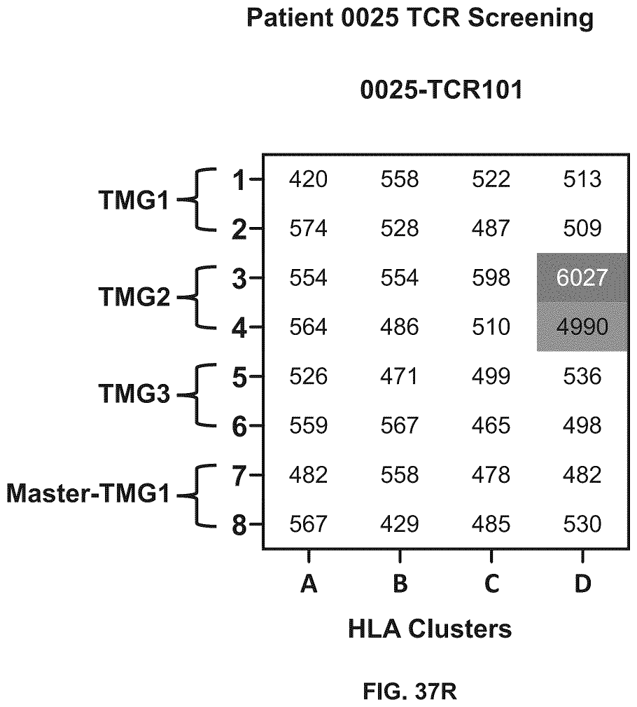

As shown in the heatmap in FIGS. 37A-R, 18 TCRs are found to be reactive to the combinations of HLA group D and either TMG2 (13 TCRs) or TMG3 (5 TCRs) in patient 0025. These reactive TCRs are clonotypes 9, 12, 30, 31, 32-1, 33-1, 36, 43-1, 45, 47, 48, 52, 62, 69, 72, 77, 87, and 101 (these correspond to the TCRs in Table 68 below). HLA cluster D contained HLA-DRA*01:01, DRB1*01:01, and DRB4*01:03.

TABLE 68

| TCR ID | TMG | HLA-Restriction |
|---|---|---|
| 0025-TCR8 | 0025-TMG2 | HLA-DRA*01:01 and DRB1*01:01 or |
| | | DRB4*01:03 |
| 0025-TCR12 | 0025-TMG2 | HLA-DRA*01:01 and DRB1*01:01 or |
| | | DRB4*01:03 |
| 0025-TCR30 | 0025-TMG2 | HLA-DRA*01:01 and DRB1*01:01 or |
| | | DRB4*01:03 |
| 0025-TCR31 | 0025-TMG2 | HLA-DRA*01:01 and DRB1*01:01 or |
| | | DRB4*01:03 |
| 0025-TCR32-1 | 0025-TMG3 | HLA-DRA*01:01 and DRB1*01:01 or |
| | | DRB4*01:03 |
| 0025-TCR33-1 | 0025-TMG3 | HLA-DRA*01:01 and DRB1*01:01 or |
| | | DRB4*01:03 |
| 0025-TCR36 | 0025-TMG3 | HLA-DRA*01:01 and DRB1*01:01 or |
| | | DRB4*01:03 |
| 0025-TCR43-1 | 0025-TMG2 | HLA-DRA*01:01 and DRB1*01:01 or |
| | | DRB4*01:03 |
| 0025-TCR45 | 0025-TMG2 | HLA-DRA*01:01 and DRB1*01:01 or |
| | | DRB4*01:03 |
| 0025-TCR47 | 0025-TMG2 | HLA-DRA*01:01 and DRB1*01:01 or |
| | | DRB4*01:03 |
| 0025-TCR48 | 0025-TMG2 | HLA-DRA*01:01 and DRB1*01:01 or |
| | | DRB4*01:03 |
| 0025-TCR52 | 0025-TMG2 | HLA-DRA*01:01 and DRB1*01:01 or |
| | | DRB4*01:03 |
| 0025-TCR62 | 0025-TMG2 | HLA-DRA*01:01 and DRB1*01:01 or |
| | | DRB4*01:03 |
| 0025-TCR69 | 0025-TMG3 | HLA-DRA*01:01 and DRB1*01:01 or |
| | | DRB4*01:03 |
| 0025-TCR72 | 0025-TMG3 | HLA-DRA*01:01 and DRB1*01:01 or |
| | | DRB4*01:03 |
| 0025-TCR77 | 0025-TMG2 | HLA-DRA*01:01 and DRB1*01:01 or |
| | | DRB4*01:03 |
| 0025-TCR87 | 0025-TMG2 | HLA-DRA*01:01 and DRB1*01:01 or |
| | | DRB4*01:03 |
| 0025-TCR101 | 0025-TMG2 | HLA-DRA*01:01 and DRB1*01:01 or |
| | | DRB4*01:03 |

### 7.7 Conclusions

The series of data described in this example illustrate the application of a high-throughput TCR isolation and screening method in a patient-derived tumor specimen wherein gene-expression data from sorted tumor infiltrating T cells are used to identify TCRs of interest for screening. Patient HLA and somatic tumor mutations are identified using NGS and bioinformatic analysis. Using a dissociated tumor sample from endometrial cancer Patient 0025, paired TCRα/β sequences are identified from tumor infiltrating T cells. Single-cell gene expression data is successfully used to cluster T cells into groups based on similar transcriptional profiles. Using the clustering, TCR sequences are identified for screening. Cluster analysis enables the identification of rare TCR sequences of potential interest. These paired TCR sequences are reconstructed in silico from which DNA expression vectors encoding sixty-one TCRs from Patient 0025 are generated. Using the TCR screening method, all sixty-one TCRs are successfully screened and eighteen TCRs are found to recognize neoantigens from the patient's tumor. The eighteen reactive TCRs, recognized a neoantigen present in either 0025-TMG2 or 0025-TMG3 when presented in the context of either HLA alleles HLA-DRA*01:01 and DRB1*01:01 or HLA-DRA*01:01 and DRB4*01:03. Overall, these data demonstrate a method for the high-throughput screening of TCRs identified from primary tumor samples. Furthermore, this example describes the identification of eighteen novel neoantigen reactive TCRs.

### Example 8: TCR Screening for Additional Patients

The series of data described in this example illustrate the application of a high-throughput TCR isolation and screening method in a patient-derived tumor specimen wherein gene-expression data from sorted tumor infiltrating T cells are used to identify TCRs of interest for screening. Patient HLA and somatic tumor mutations are identified using NGS and bioinformatic analysis. Detailed description of the methods and procedures is provided in previous examples.

Using a dissociated tumor sample from each of cancer Patients 9976, 7014, 8540, 0894, 5040, 8202, and 5239, paired TCRα/β sequences are identified from tumor infiltrating T cells. Single-cell gene expression data is successfully used to cluster T cells into groups based on similar transcriptional profiles. Using the clustering, TCR sequences are identified for screening. Cluster analysis enables the identification of rare TCR sequences of potential interest. These paired TCR sequences are reconstructed in silico from which DNA expression vectors encoding TCRs from each of Patients 9976, 7014, 8540, 0894, 5040, 8202, and 5239 are generated. Using the TCR screening method, the TCRs are successfully screened and a number of TCRs are found to recognize neoantigens from each of the patient's tumor. Sequences of representative reactive TCRs identified from these patient samples are provided in Tables 26-64. Sequences of additional representative reactive TCRs identified from other patient samples are provided in Tables 65-79.

Additional KRAS neoantigen specific TCR screening process is described below.

### KRAS Neoantigen Specific TCR Screening Process

On Day 1, COS-7 cells are seeded at 20,000 per well (96 multiwells) overnight (in 37° C. incubator).

On Day 2, cell medium is replaced with antibiotic free DMEM medium before transfection. 150 ng of Master tandem minigene (MasterTMG) and 300 ng HLA plasmids are transfected using lipofectamine 2000. Master TMG contains KRAS G12C, KRAS G12D, KRAS G12R and KRAS G12V mutations. 3-4 HLA plasmids (75-100 ng each) are transfected together in one well to enhance screening efficacy. Total 5-6 groups of HLA are transfected for each patient. Each condition includes one or two HLA types including A, B, C, DP, DQ and DR. 25 μls of OptiMEM medium are used to dilute either DNA plasmids (450 ng total) or Lipofectamine (0.6 μl) for each well. DNA tube (A) or lipofectamine tube (B) are mixed well separately and incubated for 5 minutes at room temperature (RT). Tube B is then added to tube A, and the mixture is incubated for 20 minutes at RT. Transfection mix (50 μls) is added to each well and cells are cultured overnight in 37° C. incubator. Jurkat NFAT reporter cells are counted and seeded at 1 million/ml with fresh RPMI1640 complete medium overnight to enhance electroporation efficacy (10% FBS and 1% Pen/Strep).

On Day 3, NEON transfection system is set up in the Biosafety Cabinet (BSC) with program 1,325v, 10 mins, 3 Pulse. 2 mls of RPMI without Pen/Strep are added into 24 multiwells and labeled with corresponding mTCR number. Multiwells are pre-warmed in 37° C. incubator while preparing EP. Jurkat NFAT cells are spined down at 100 g for 10 minutes at room temperature. Cells are washed with PBS and cell numbers are measured with NC3000. 6 million Jurkat NFAT cells are loaded into 15 ml conical tubes and spined down at 100 g for 10 minutes at room temperature. During centrifugation, Buffer R (130 μls each) is prepared in eppendorfs and Electrolytic Buffer E2 (3 mls each) is aliquoted in NEON tubes. 13 μls of TCR plasmids (2 mg/ml) is added to corresponding eppendorfs containing Buffer R and mixed well. The mixture of DNA and Buffer R is loaded to the NEON tubes using NEON tips. If EP is successful, “COMPLETE” should show on the screen in a few seconds after “START” is clicked. Buffer R/DNA mixture is transferred immediately in 24 multiwells containing antibiotic free RPMI medium. H57 antibody is utilized to coat plate (1 μg/ml, 250 μl/well) overnight to measure EP efficacy next day. Peptide is prepared at 50 mg/ml and pulsed at 1 μg/ml to increase the sensitivity of class II TCR reactivity during first round TCR screening and second round parsing for confirming KRAS neoantigen specificity.

On Day 4, Jurkat NFAT-mTCR cells are seeded (100 μl/well) on top of transfected COS-7 cells for 4-5 hours. As control, Jurkat NFAT-mTCR cells are also plated on H57 coated plate to perform mTCR functional test. After 4-5 hours incubation, cells from 96 multiwells are transferred to U bottom plates and spined down at 400 g for 5 minutes. Cells are then lysed with 1× passive lysis buffer (100 μl/well) for 15 minutes on an orbital shaker at RT. 50 μls cell lysis are loaded onto OPTIPLATE as well as 100 μl of Promega Luciferase substrate. Luciferase activity is measured immediately with BioTek reader. Jurkat NFAT cells mTCR expression is measured with flow cytometry using antibody cocktail CD3, CD4, CD8A, CD8B and H57. HLA expression of COS-7 cells is measured with flow cytometry using antibody cocktail HLA-A2, HLA-PanA, HLA-DP, HLA-DQ and HLA-DR.

KRAS Q61H TCR-T Cell Generation from Healthy Donor PBMCs

PBMC cells are thawed, spun down, resuspended in electroporation buffer together with TCR plasmids, and electroporated. Following electroporation, cell suspensions are collected, transferred to recovery media (50:50 media), and incubated in a 37° C./5% CO₂ incubator overnight. Within 24 hours post-electroporation (Day 1), live cells are transferred to G-REX® culture plates and incubated with a first expansion media (50:50 media containing 300 IU/ml of IL-2+30 ng/ml of IL-21+T Cell TransAct™). Cells are fed regularly with cytokines. After 10 days of first phase expansion, TCR+ cells are isolated with mTCR antibody. The isolated TCR+ T cells are transferred to G-REX® culture plates and incubated with a second expansion media (50:50 media containing 3000 IU/ml of IL-2+T Cell TransAct™). Cells are fed regularly with cytokines. After 19 days of second phase expansion, cells are harvested, and the various assays are performed.

### Results

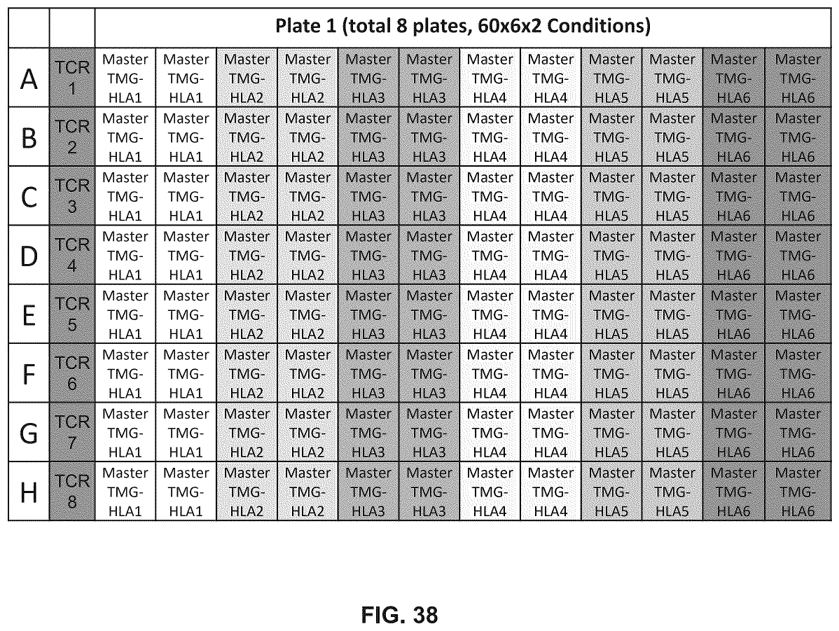

As shown in FIG. 38, eight TCRs from Patient 9976 are screened. Each TCR occupies one row on the 96 multiwell for coculture. 6 HLA groups are co-transfected with Master TMG. Every condition is in duplicates. Similarly, TCRs from Patient 7014 are also screened. 5 HLA groups are transfected from Patient 7014.

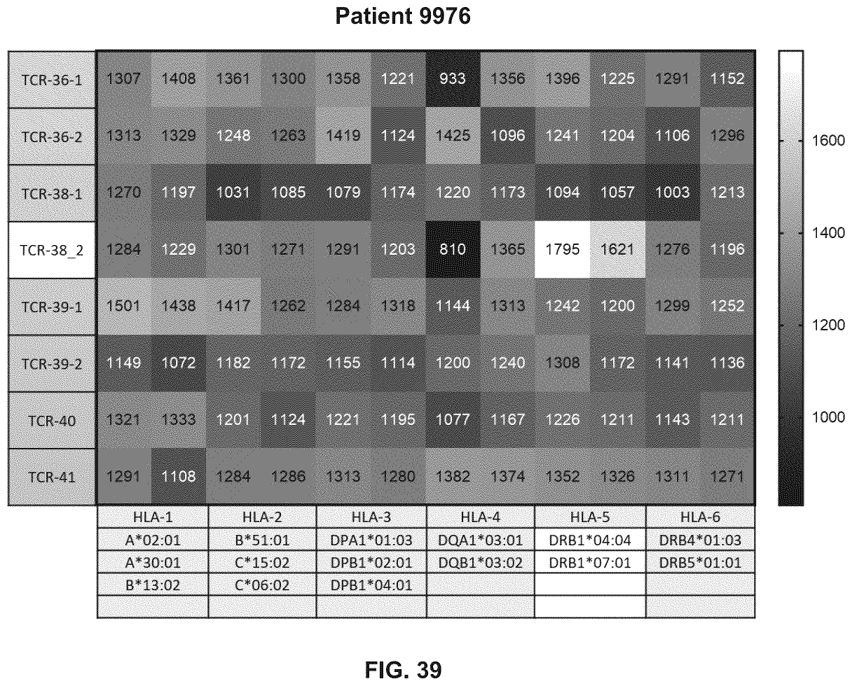

FIG. 39 shows that TCR 38-2 from Patient 9976 is specific to HLA DRB1*04:04 or DRB1*07:01. On Day 1 of this experiment, COS-7 cells are seeded at 20,000 per well overnight in 96 multiwells. On Day 2, COS-7 cells are transfected in each well with 150 ng of Master TMG+300 ng of HLAs (75-100 ng each). On Day 3, NEON transfection system is set up the following day and 5 million cells were electroporated with each of the TCR plasmid and negative control (NTC). On Day 4, Jurkat cells are harvested and seeded on top of transfected COS-7 cells. After 4-5 hours co-culture, cells are harvested, and luciferase activity is measured. There are 60 TCRs picked from 10× single cell sequencing for this patient 9976. In addition, this patient has 2 HLA-A, 2 HLA-B, 2 HLA-C, 1 HLA-DQ-A, 1 HLA-DQ-B, 1 DP-A, 2 DP-B, 2 DRB1,1 DRB4 and 1 DRB5. The HLA plasmids are separated into 6 groups (HLA A&B, HLA B&C, HLA DP, HLA DQ and 2 HLA DR) to reduce the number of combinations with TMG plasmids.

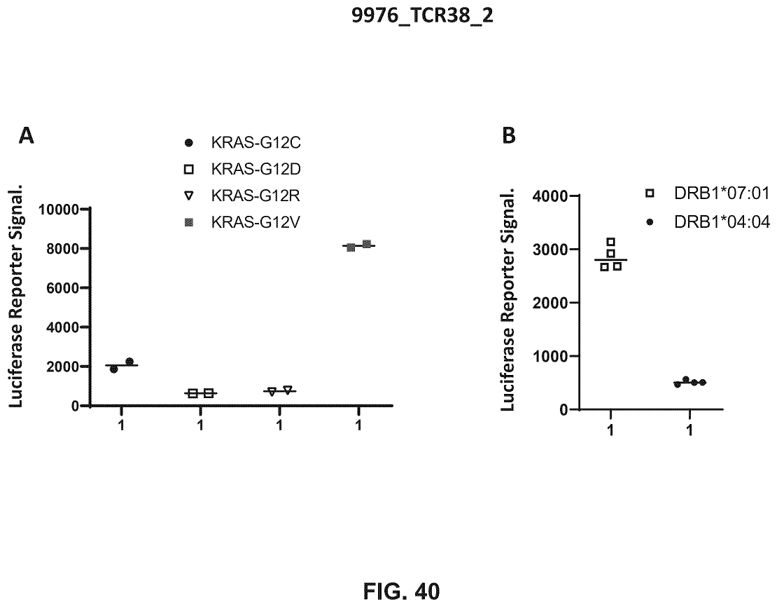

To further address the mutation and HLA allele specificity, KRAS peptides are pulsed individually at 1 μg/ml or COS-7 cells are transfected with individual HLA and Master TMG. As shown in FIG. 40, TCR38-2 from Patient 9976 is identified as being reactive to the KRAS-G12V mutation (panel A) and DRB1*07:01 is its specific HLA (panel B).

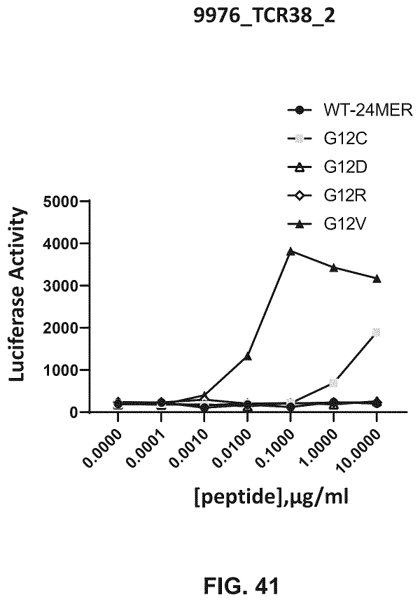

The KRAS peptides as well as the wide type (WT) peptide are also titrated using Jurkat-NFAT & COS-7 system. As shown in FIG. 41, TCR38-2 from Patient 9976 is reactive to KRAS.G12V at concentration as low as 1 ng/ml, suggesting higher avidity of the TCR. In addition, TCR38-2 shows relatively weak reactivity against KRAS.G12C peptide but not with G12D or G12R, suggesting that this TCR might be used to treat more than one mutation.

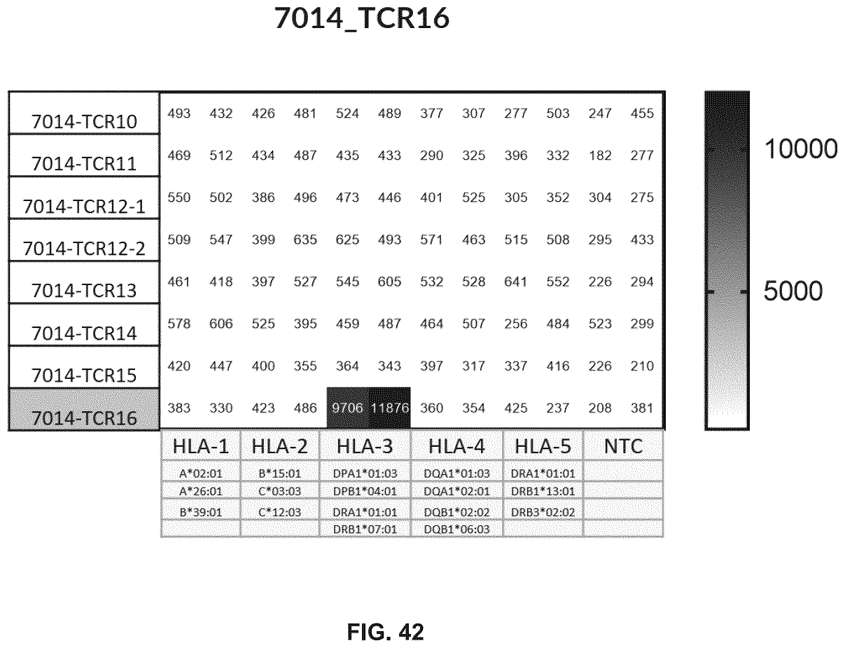

FIG. 42 shows that TCR 16 from Patient 7014 is specific to HLA DPA1*01:03& DPB1*04:01 or DRB1*07:01. On Day 1 of this experiment, COS-7 cells are seeded at 20,000 per well overnight in 96 multiwells. On Day 2, COS-7 cells are transfected in each well with 150 ng of Master TMG+300 ng of HLAs (75-100 ng each). On Day 3, NEON transfection system is set up the following day and 5 million cells are electroporated with each of the TCR plasmids from patient 7014 and negative control (NTC). On Day 4, Jurkat cells are harvested and seeded on top of transfected COS-7 cells. After 4-5 hours co-culture, cells are harvested, and luciferase activity is measured. There are 60 TCRs picked from 10× single cell sequencing for this patient 7014. In addition, this patient had 2 HLA-A, 2 HLA-B, 2 HLA-C, 2 HLA-DQ-A, 2 HLA-DQ-B, 1 DP-A, 1 DP-B, 2 DRB1, and 1 DRB3. The HLA plasmids are separated into 5 groups (HLA A&B, HLA B&C, HLA DP/DR, HLA DQ and HLA DR) to reduce the number of combinations with TMG plasmids.

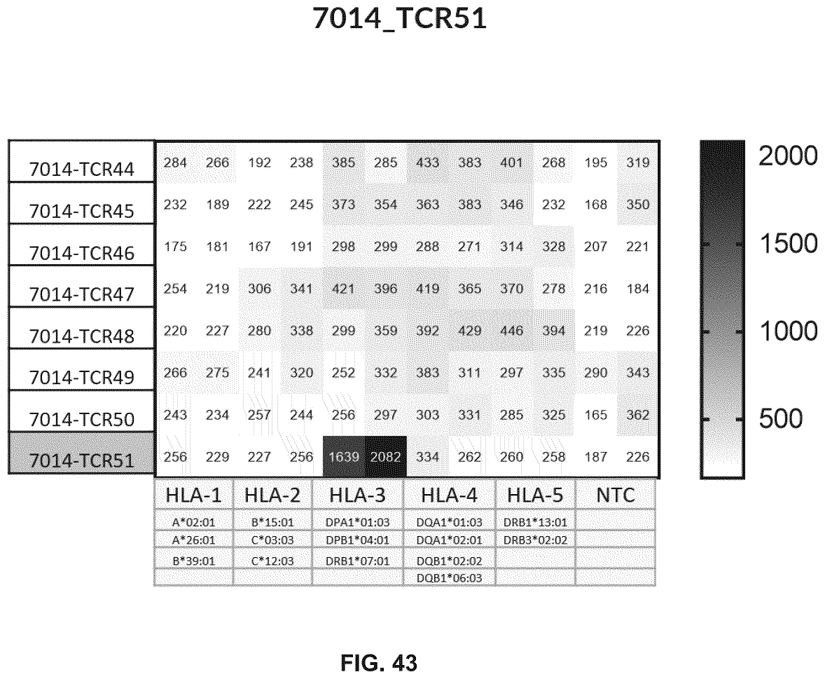

As shown in FIG. 43, TCR 51 from Patient 7014 is specific to HLA DPA1*01:03& DPB1*04:01 or DRB1*07:01.

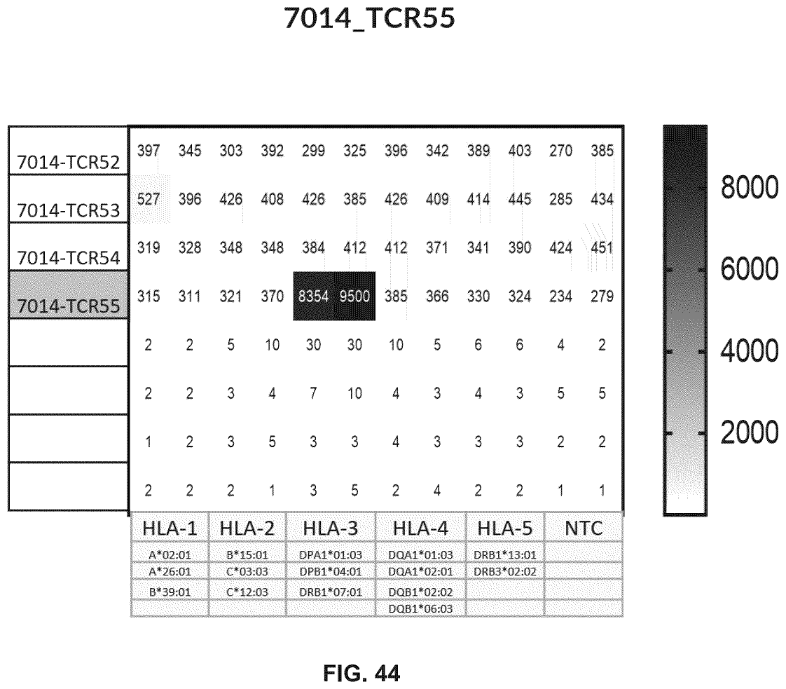

As shown in FIG. 44, TCR 55 from Patient 7014 is specific to HLA DPA1*01:03& DPB1*04:01 or DRB1*07:01.

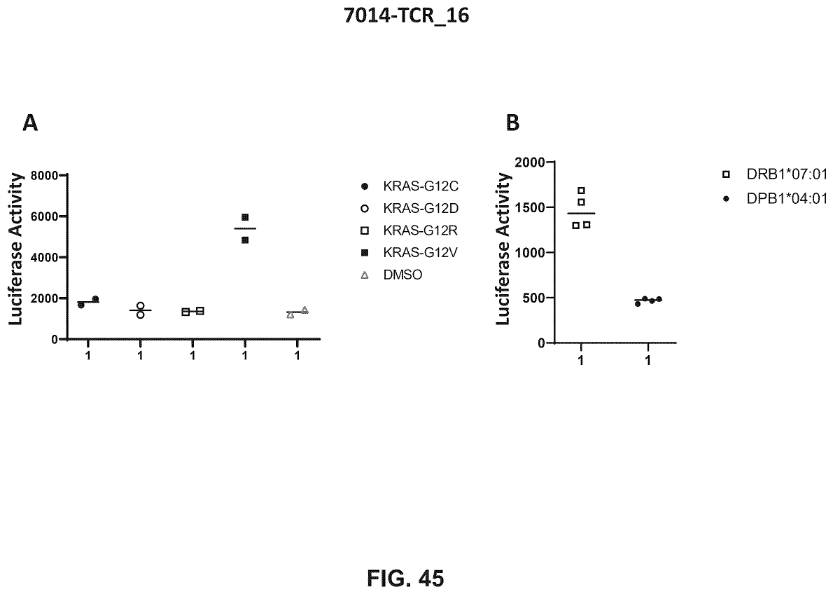

To further address the mutation and HLA allele specificity, KRAS peptides are pulsed individually at 1 μg/ml or COS-7 cells are transfected with individual HLA and Master TMG. As shown in FIG. 45, TCR16 from Patient 7014 is identified as being reactive to the KRAS.G12V mutation (panel A) and DRB1*07:01 is its specific HLA (panel B).

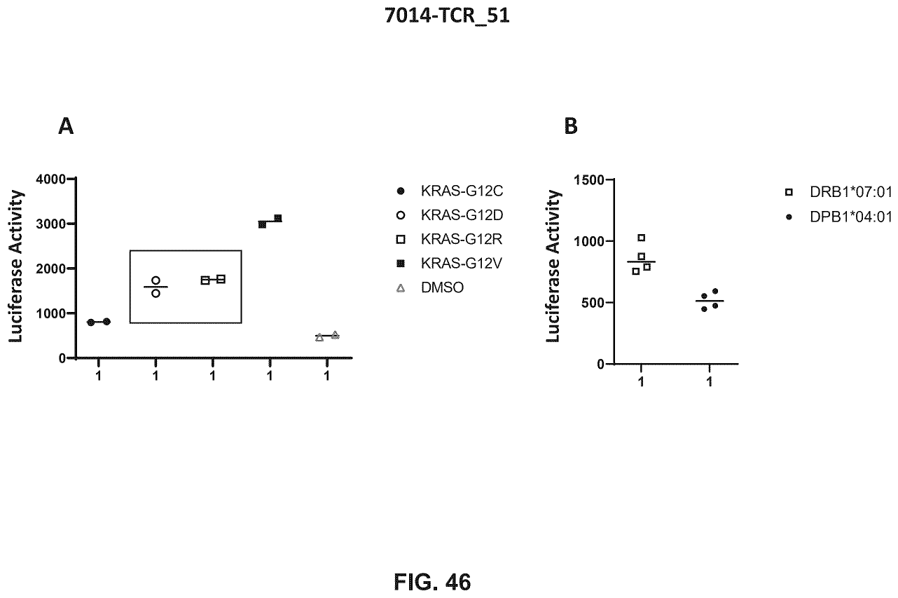

As shown in FIG. 46, TCR 51 from Patient 7014 is identified as being reactive to the KRAS.G12V mutation (panel A) and DRB1*07:01 is its specific HLA (panel B). In addition, TCR 51 shows relatively weak reactivity against KRAS.G12D and G12R peptide but not with G12C, suggesting that this TCR might be used to treat more than one mutation.

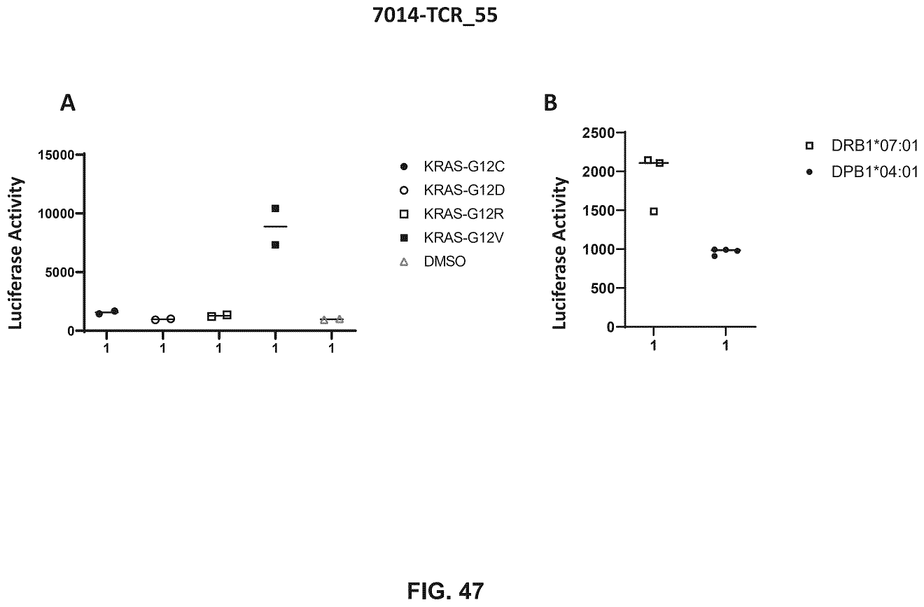

As shown in FIG. 47, TCR 55 from Patient 7014 is identified as being reactive to the KRAS.G12V mutation (panel A) and DRB1*07:01 is its specific HLA (panel B).

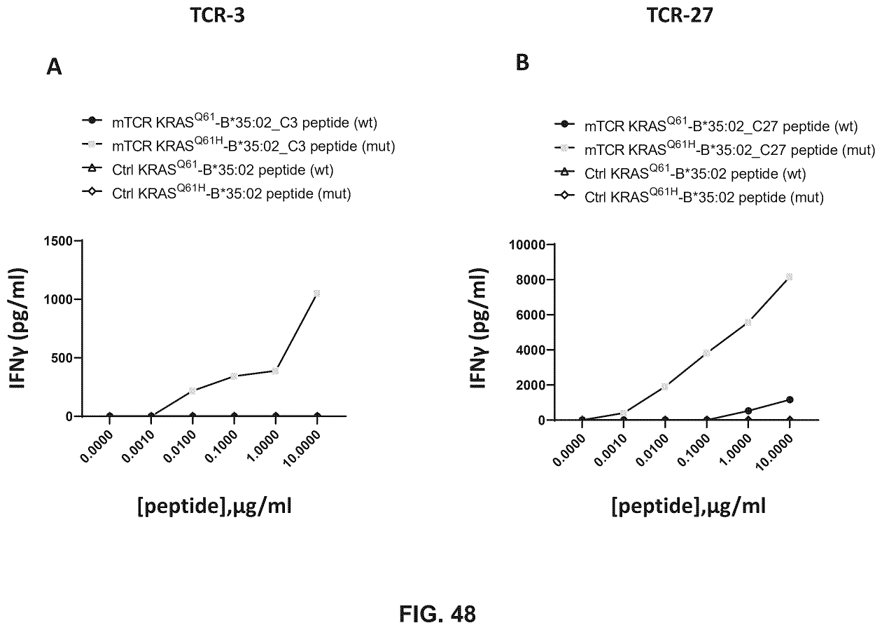

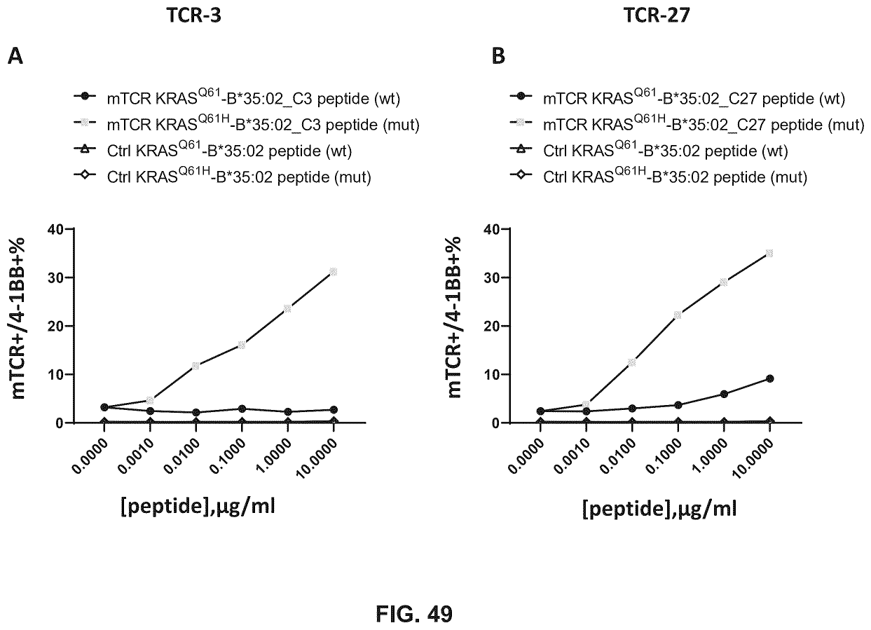

To test the specificity of the TCRs, TCR-T cells are co-cultured with matched antigen presenting cells or dendritic cells (DCs) expressed HLA B*35:02. DCs are pulsed with KRAS.Q61H in wild type or mutated variants for 2 hours. Expression of T-cell activation is measured by up-regulation of interferon gamma (IFNγ) in the secreted supernatant. As shown in FIG. 48, dose response to the mutated, but not the wild type, is observed for both TCR3 (panel A) and TCR27 (panel B) from Patient 8434, demonstrating that TCR-T cells are specific and do not recognize the germline sequences and are, therefore, unlikely to recognize normal tissues. Expression of T-cell activation is also measured by up-regulation of surface marker 4-1BB. As shown in FIG. 49, dose response to the mutated, but not the wild type, was observed for both TCR3 (panel A) and TCR27 (panel B), demonstrating that TCR-T cells are specific and do not recognize the germline sequences and are, therefore, unlikely to recognize normal tissues.

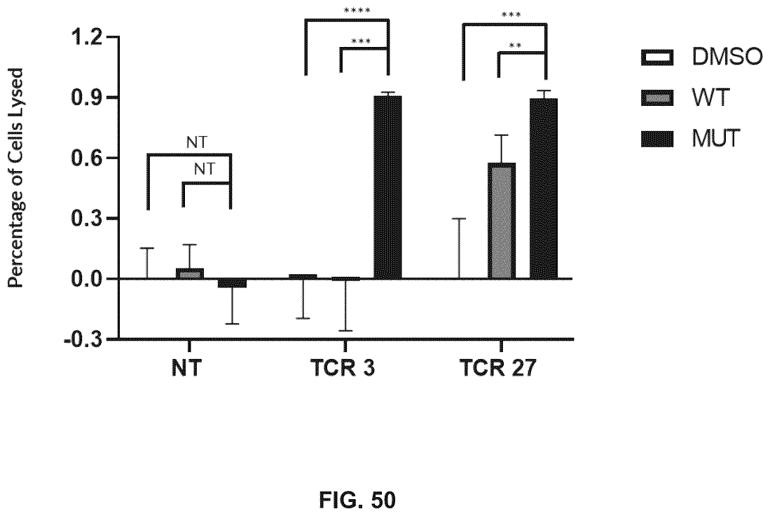

Furthermore, to test tumor killing by neoantigen-reactive TCR, tumor cells are pulsed with 1 μg/ml KRAS.Q61H peptide, wide type peptide, or DMSO, and co-cultured with open repertoire untransfected T cells (NT) or TCR-T cells (TCR3 or TCR27). Tumor killing is evaluated by CellTiter-Glo assay which evaluates viable cells relative to control wells and is used to calculate relative specific lysis. Multiple T test is performed for statistic analysis. FIG. 50 demonstrates specific recognition of the tumor cells with matching HLA and mutation, by TCR-T cells and not untransfected T cells, indicating that specific tumor killing could occur through this approach.

Overall, these data demonstrate a method for the high-throughput screening of neoantigen reactive TCRs identified from primary tumor samples. Furthermore, this example describes the identification of additional novel neoantigen reactive TCRs.

### Example 9. Expression of TCRs in Recombinant Vectors and Evaluation of MBIL-15 TCR-T Cells Reactivation Potential

To improve homogeneity of multigene co-expression and product manufacturability, recombinant nucleic acid SB transposon plasmids comprising polycistronic expression cassettes are constructed. The polycistronic expression cassettes each include a transcriptional regulatory element operably linked to a polycistronic polynucleotide that encodes a TCR α chain of any TCR sequences disclosed in Tables 1-79 (referred to herein as “TCRα” or “A”), a TCR β chain of any TCR sequences disclosed in Tables 1-79 (referred to herein as “TCRβ” or “B”), and membrane-bound IL-15/IL-15Rα fusion protein (referred to herein as “mbIL15” or “15”), each separated by a furin recognition site and either a P2A element or a T2A element that mediates ribosome skipping to enable expression of separate polypeptide chains.

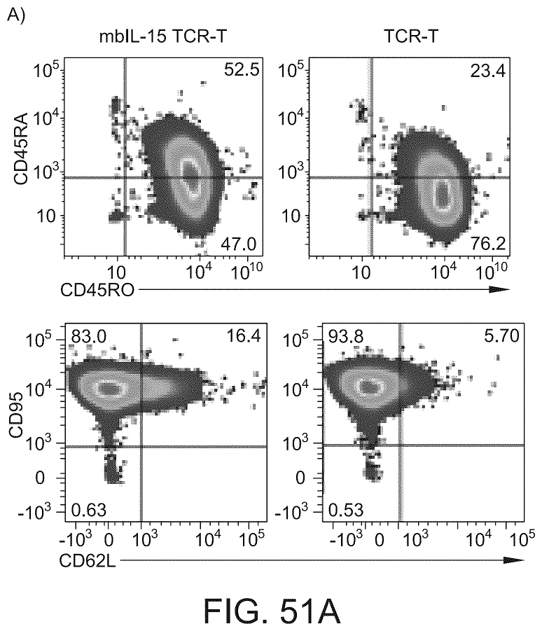

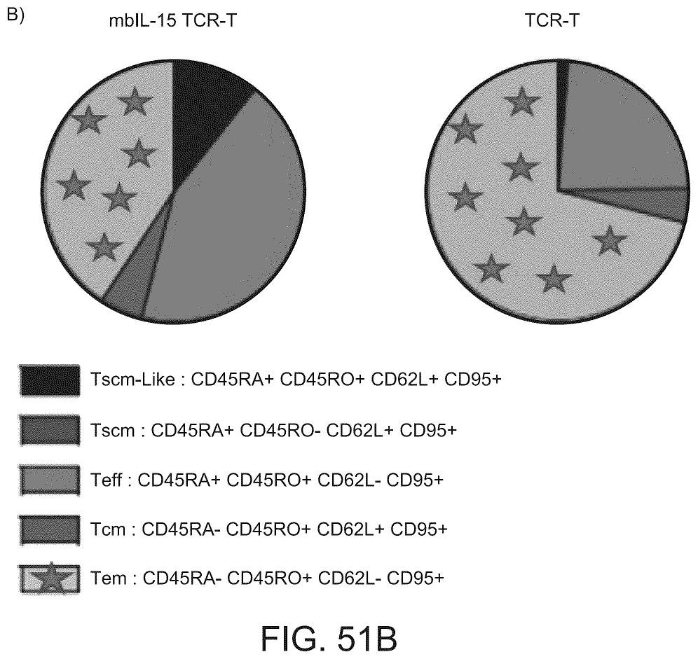

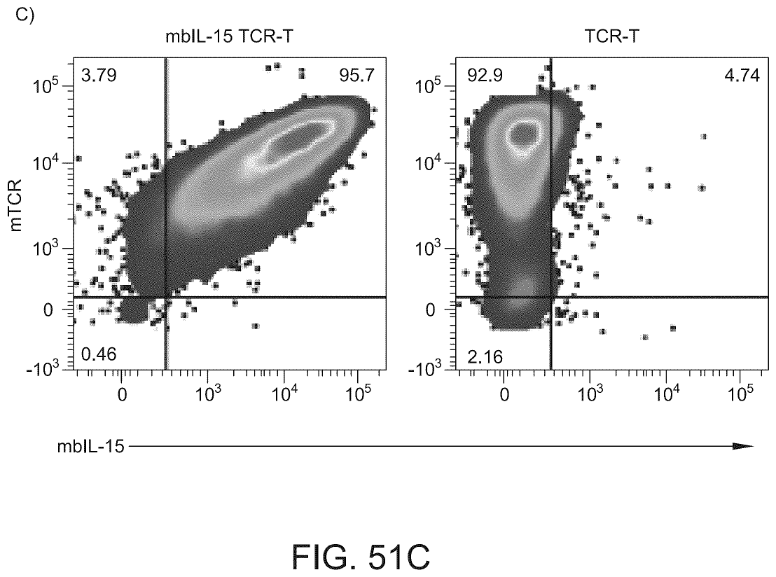

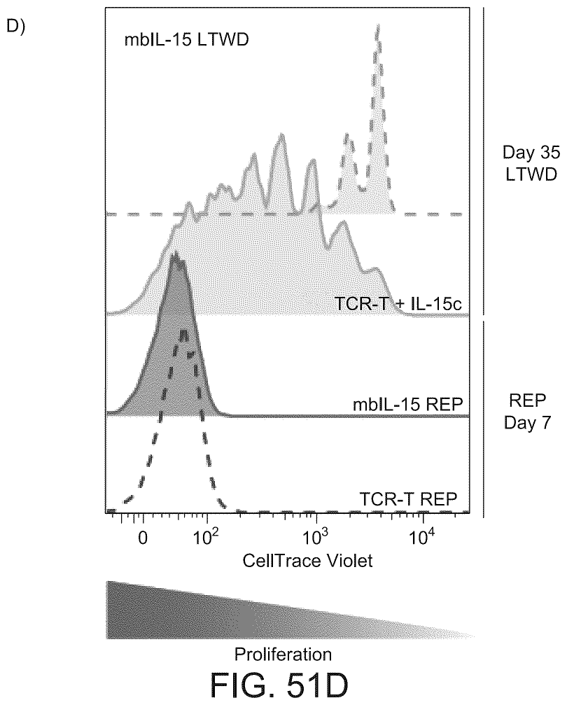

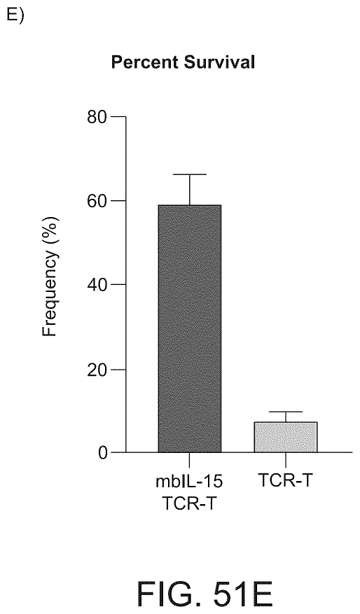

In one experiment, the reactivation of TCR-T cells expressing mbIL-15 (mbIL-15 TCR-T cells) is performed after long-term cytokine withdrawal (LTWD) to determine effector T cells phenotype. Briefly, TCR-T cells were cultured with IL-15 complex (IL-15c) and mbIL-15 TCR-T cells from 35 day LTWD cultures are restimulated for 7 days with irradiated feeder cells, IL-2 and anti-CD3 antibody. Pseudocolor plots show the expression of CD45RA and CD45RO (upper plots), CD95 and CD62L (lower plots) (FIG. 51A), and the expression of mTCR and mbIL-15 in mbIL-15 TCR-T and TCR-T cells (FIG. 51C). Pie charts show that TCR-T cells expressing mbIL-15 differentiated into four main subsets: Tscm-like, Teff, Tcm, and Tem (FIG. 51B). TCR-T cells cultured with IL-15 complex (IL-15c) differentiate into a variation of the same four main subsets. The data shows that when the mbIL-15 TCR-T cells are restimulated, those that have previously become Tscm cells gave rise to effector cell subsets. Histograms show CellTrace Violet dilution in CD3+ T cells (FIG. 51D). T cells from representative cultures are cultured for an additional 4 weeks with or without cytokines (LTWD). Bar graphs show the percent survival of CD3+ T cells (FIG. 51E). These results suggest that mbIL-15 TCR-T cells are able to maintain their reactivation potential and were able to differentiate into Teff and Tem cells upon re-stimulation.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes. Other embodiments are within the following claims.

1. A method for identifying a neoantigen-reactive T cell receptor (TCR), comprising:
i) co-culturing
a) a reporter T cell comprising a TCR expression cassette, and
b) an antigen presenting cell (APC) that expresses a target neoantigen sequence and a matched human leukocyte antigen (HLA) sequence; and
ii) identifying a positive reporter signal in the reporter T cell to identify a neoantigen-reactive TCR.

2. A method for identifying a neoantigen-reactive TCR, comprising:
i. obtaining single-cell gene expression profiles from a population of tumor infiltrating lymphocytes (TIL) isolated from a patient sample,
ii. performing bioinformatics analyses on the single cell gene expression data to identify TCR clonotypes of interest,
iii. creating recombinant TCR sequences;
iv. preparing a reporter T cell comprising a TCR expression cassette encoding a TCR sequence reconstructed from paired TCR α and β chain sequences identified from the clonotypes of interest in step ii,
v. preparing a tandem minigene (TMG) expression vector;
vi. analyzing the patient sequencing data to identify class I and class II HLA alleles and preparing HLA expression vectors comprising the class I HLA and class II HLA allele sequences;
vii. preparing an antigen presenting cell (APC) comprising transfecting the TMG expression vector and one or more HLA expression vectors into a cell wherein each transfection condition comprises a TMG and one or two HLA types;
viii. co-culturing the reporter T cell in step iii with the APC of step vii,
ix. identifying positive reporter activity in the reporter T cell to identify a neoantigen-reactive TCR.

3. The method of claim 2, further comprising obtaining whole exome sequence (WES) data from the patient sample and analyzing the WES data to identify class I and class II HLA alleles.

4. The method of claim 2, wherein the performing bioinformatics analysis further comprises clustering the TCR clonotypes and to select a clonotype of interest.

5. The method of claim 2, wherein the creating recombinant TCR sequences comprises designing alpha and beta TCR sequences in silico.

6. The method of claim 2, wherein the TMG comprises nucleic acid sequences for the expression of concatenated amino acid sequences of non-synonymous single nucleotide variants (SNVs)

7. The method of claim 2, wherein the clustering comprises grouping the TCR clonotype by CD8 or CD4 expression, gene function of differentially expressed genes, and the level of expression of each TCR.

8. The method of claim 2, further comprising culturing the reporter T cell having the identified neoantigen-reactive TCR with one or more peptides representing the non-synonymous single nucleotide variants (SNVs) present in the TMG.

9. The method of claim 1, wherein the APC is a COS-7 cell.

10. The method of claim 1 or 2, wherein the reporter T cell is an immortalized T cell line.

11. The method of claim 10, wherein the immortalized T cell is a Jurkat cell.

12. The method of claim 11, wherein the Jurkat cell is a Jurkat NFAT cell.

13. The method of any one of claims 1 to 12, wherein the TCR expression cassette comprises a TCR sequence reconstructed from TCR α and β chain sequences identified from tumor infiltrating lymphocytes (TILs) isolated from a tumor sample, and wherein the target neoantigen sequence and the matched HLA sequence are identified from the same tumor sample.

14. The method of any one of claims 1 to 13, wherein the method comprises identifying TCR sequences from TILs isolated from a tumor sample.

15. The method of claim 7, wherein the method further comprises identifying somatic mutations in the tumor sample and determining the germline HLA typing of the tumor sample.

16. A method of identifying a neoantigen-reactive T cell receptor (TCR), comprising:
i) obtaining TCR α and β chain sequences from tumor infiltrating lymphocytes (TILs) isolated from a patient sample;
ii) obtaining neoantigen sequences comprising somatic mutations present in the tumor sample, and the germline HLA typing of the patient sample;
iii) co-culturing
a) a reporter T cell expressing a TCR sequence reconstructed from the TCR α and β chain sequences obtained in step i), and
b) an antigen presenting cell (APC) that expresses a neoantigen sequence and a matched human leukocyte antigen (HLA) sequence obtained in step ii; and
iv) evaluating the reporter activity in the reporter T cell to identify a neoantigen-reactive TCR.

17. The method of claim 16, wherein the APC is a COS-7 cell, and the reporter T cell is a Jurkat NFAT cell.

18. The method of claim 13-17, wherein the isolated TILs are first expanded ex vivo and then co-cultured with APCs modified to express relevant HLA alleles and antigens obtained from the tumor sample.

19. The method of any one of claims 1 to 18, wherein the reporter T cell comprises a reporter system that is activated by the binding of the TCR to the neoantigen.

20. The method of any one of claims 1 to 19, wherein the reporter T cell and the APC are co-cultured in a ratio of 1:16, 1:8, 1:4, 1:2, 1:1, 2:1, 4:1, 8:1, or 16:1.

21. The method of any one of claims 1 to 20, wherein the reporter T cell and the APC are co-cultured for 1-48 hours.

22. A TCR, or an antigen-binding portion thereof, isolated according to the method of any one of claims 1 to 21.

23. A TCR, or an antigen-binding portion thereof, comprising a sequence selected from the group consisting of SEQ ID NOs: 1-300, 536-1003, and 1025-1204.

24. A neoantigen/HLA complex, wherein the neoantigen comprises a sequence selected from the group consisting of SEQ ID NOs: 310 to 535 and wherein the HLA comprises a sequence selected from a group consisting of SEQ ID NOs: 301 to 309.

25. The neoantigen/HLA complex of claim 24, wherein the neoantigen is SEQ ID NO: 481 and the HLA sequence is SEQ ID NOs: 303 and 305 or SEQ ID NOs: 304 and 305.

26. The neoantigen/HLA complex of claim 24, wherein the neoantigen is SEQ ID NO: 385 and the HLA sequence is SEQ ID NO: 301.

27. The neoantigen/HLA complex of claim 24, wherein the neoantigen is SEQ ID NO: 394 and the HLA sequence is SEQ ID NOs: 308 and 306.

28. The neoantigen/HLA complex of claim 24, wherein the neoantigen is SEQ ID NO: 405 and the HLA sequence is SEQ ID NO: 302.

29. A recombinant vector expressing the T cell receptor, or an antigen-binding portion thereof, of claim 22 or 23.

30. A polynucleotide encoding an amino acid sequence that is at least 90%, 91%, 92%, 93%, 94%, 95%, 96%, 97%, 98%, 99%, or 100% identical to an amino acid sequence selected from the group consisting of SEQ ID NOs: 1-300, 536-1003, and 1025-1204.

31. A population of cells that comprise the recombinant vector of claim 29, or the polynucleotide of claim 30.

32. The population of cells of claim 31, wherein the recombinant vector or the polynucleotide is integrated into the genome of the population of cells.

33. The population of cells of claim 31 or 32, wherein the cells are immune effector cells.

34. The population of cells of claim 33, wherein the immune effector cells are selected from the group consisting of T cells, natural killer (NK) cells, B cells, mast cells, and myeloid-derived phagocytes.

35. A pharmaceutical composition comprising the population of cells of any one of claims 31 to 34, and a pharmaceutically acceptable carrier.

36. A method of preparing a medicament for the treatment or prevention of a medical condition, the method comprising preparing the population of cells any one of claims 31 to 34.

37. A method of treating a disease or medical condition, the method comprising administering the pharmaceutical composition of claim 23 to a patient in need.

38. The method of claim 36 or 37, wherein the disease or medical condition is a cancer.

39. A co-culture reporter system for identifying a neoantigen-reactive T cell receptor (TCR), comprising:
i) a reporter T cell comprising a TCR expression cassette, co-cultured with
ii) an antigen presenting cell (APC) that expresses a target neoantigen sequence and a matched human leukocyte antigen (HLA) sequence.

40. The co-culture reporter system of claim 39, wherein the APC is a COS-7 cell, and the reporter T cell is a Jurkat NFAT cell.

41. The method of claims 1 to 38, or the reporter system of claim 39 or 40, wherein the target neoantigen is expressed in an antigen encoding plasmid.

42. The method or the reporter system of claim 41, wherein the antigen encoding plasmid is a Tandem Minigene (TMG) plasmid.

43. The method of claims 1 to 38, or the reporter system of claim 39 or 40, wherein the target neoantigen is introduced to the APC by the pulsing of peptide pools.

44. The method of claims 1 to 38, or the reporter system of claim 39 or 40, wherein the reporter T cell is a primary T cell.

45. The method of claims 1 to 38, or the reporter system of claim 39 or 40, wherein the reporter cell is from an immortalized T cell line.

46. The method of claims 1 to 38, or the reporter system of claim 39 or 40, wherein the TCR expression cassette comprises a full-length TCR sequence.

47. The method of claims 1 to 38, or the reporter system of claim 39 or 40, wherein the reporter T cell expresses any or all protein components of the TCR signaling complex or downstream signaling components.

48. The method of claims 1 to 38, or the reporter system of claim 39 or 40, wherein the reporter T cell expresses one or more components selected from the group consisting of CD3, CD4, CD8a, and CD8b.

49. The method or the reporter system of claim 48, wherein the reporter T cell is modified to enhance the activity of the one or more protein components.

50. The method of claims 1 to 38, or the reporter system of claim 39 or 40, wherein the TCR expression cassette is cloned into a non-viral gene transfer vector.

51. The method or the reporter system of claim 50, wherein the TCR expression cassette is cloned into a transposon.

52. The method of claims 1 to 38, or the reporter system of claim 39 or 40, wherein the APC is a classical professional APC (such as DC).

53. The method of claims 1 to 38, or the reporter system of claim 39 or 40, wherein the APC is an artificial APC.

54. The method of claims 1 to 38, or the reporter system of claim 39 or 40, wherein the APC endogenously express an HLA allele.

55. The method of claims 1 to 38, or the reporter system of claim 39 or 40, wherein the APC comprises a transgenic HLA expression plasmid.

56. The method of claims 1 to 38, or the reporter system of claim 39 or 40, wherein the APC expresses multiple transgenic HLA alleles in a single cell.

57. The method of claims 1 to 38, or the reporter system of claim 39 or 40, wherein the APC expresses a co-stimulatory molecule.

58. The method or the reporter system of claim 57, wherein the co-stimulatory molecule is one or more selected from the group consisting of 4-1BBL, CD40, CD80, CD86, or OX40L.

59. The method of claim 18, wherein a gene signature for identifying neoantigen reactive TCRs from ex vivo expanded TIL includes one or more gene(s) selected from the group consisting of XCL2, XCL1, IL2, CSF2, IFNG, CCL4, CCL4L2, TNF, CCL3, RGCC, TNFSF9, DUSP2, NFKBID, MIR155HG, NR4A3, EVI2A, CRTAM, ZBED2, FABP5, PIM3, NR4A1, IL10, TNFSF14, NR4A2, LINC00892, ZFP36L1, GZMB, MYC, SPRY1, KDM6B, EGR2, PHLDA1, PPP1R2, VSIR, REL, PRDX1, SLA, CYTOR, DDX21, IER3, PGAM1, NAMPT, HSP90AB1, IL23A, FAM107B, BCL2A1, ZEB2, ZBTB32, BTG2, GADD45B, RILPL2, SEMA7A, TGIF1, SRGN, RAN, CFLAR, MAT2A, SIAH2, PRNP, RNF19A, FASLG, NME1, EVI2B, HSPH1, NOP16, CSRNP1, and TAGAP.

60. A recombinant vector comprising a polycistronic expression cassette, wherein the polycistronic expression cassette comprises a transcriptional regulatory element operably linked to a polycistronic polynucleotide that comprises:
a. a first polynucleotide sequence that encodes a T cell receptor (TCR) alpha chain comprising an alpha chain variable (Vα) region and an alpha chain constant (Cα) region;
b. a second polynucleotide sequence that comprises a first 2A element;
c. a third polynucleotide sequence that encodes a TCR beta chain comprising a beta chain variable (Vβ) region and a beta chain constant (Cβ) region;
d. a fourth polynucleotide sequence that comprises a second 2A element; and
e. a fifth polynucleotide sequence that encodes a fusion protein that comprises IL-15, or a functional fragment or functional variant thereof, and IL-15Rα, or a functional fragment or functional variant thereof.

61. The recombinant vector of claim 60, wherein the polycistronic polynucleotide comprises the first, the second, the third, the fourth, and the fifth polynucleotide sequence in any order from 5′ to 3′.

62. The recombinant vector of claim 60 or 61, wherein the TCR alpha chain comprises an amino acid sequence at least 90%, 91%, 92%, 93%, 94%, 95%, 96%, 97%, 98%, 99%, or 100% identical to the amino acid sequence of TCR alpha chain sequences disclosed in Tables 1-79, and wherein the TCR beta chain comprises an amino acid sequence at least 90%, 91%, 92%, 93%, 94%, 95%, 96%, 97%, 98%, 99%, or 100% identical to the amino acid sequence of TCR beta chain sequences disclosed in Tables 1-79.

63. A population of cells that comprise the recombinant vector of any one of claims 60-62.
